(19) United States
(12) Patent Application Publication
(10) Pub. No.: US 2025/0199015 A1
(43) Pub. Date: Jun. 19, 2025

(54) CYCLIN-DEPENDENT KINASE 2 INHIBITORS FOR MEDICAL TREATMENT

(71) Applicant: INCYCLIX BIO, LLC, Research Triangle Park, NC (US)

(72) Inventors: Jay Copeland Strum, Hillsborough, NC (US); John E. Bisi, Apex, NC (US); Alec Gibson Trub, Durham, NC (US)

(73) Assignee: INCYCLIX BIO, LLC, Research Triangle Park, NC (US)

(21) Appl. No.: 18/990,892

(22) Filed: Dec. 20, 2024

Related U.S. Application Data
(63) Continuation of application No. PCT/US2023/025791, filed on Jun. 20, 2023.
(60) Provisional application No. 63/353,729, filed on Jun. 20, 2022.
(60) Provisional application No. 63/444,523, filed on Feb. 9, 2023.
(60) Provisional application No. 63/460,201, filed on Apr. 18, 2023.
(60) Provisional application No. 63/470,621, filed on Jun. 2, 2023.

(51) Int. Cl. G01N 33/68; A61K 31/519; A61P 35/00

(52) U.S. Cl. CPC G01N 33/6893; A61K 31/519; A61P 35/00; G01N 2333/4739; G01N 2800/52

(57) ABSTRACT

Use of a pyrimidine-based selective cyclin-dependent kinase 2 (CDK2) of structure:

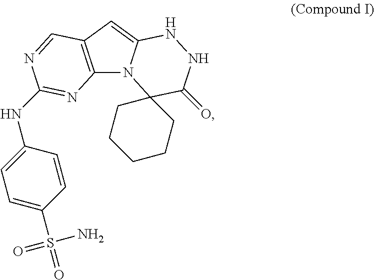 [SMILES: NS(=O)(=O)c1ccc(Nc2ncc3cc4n(c3n2)C2(CCCCC2)C(=O)NN4)cc1]

or a pharmaceutically acceptable salt thereof or morphic form described herein for the treatment of disorders characterized by aberrant cellular division having amplified cyclin E activation and/or expression or aberrant cellular division disorders, CDK4/6 resistance, and/or endocrine therapy resistance, including but not limited to the treatment of tumors and cancers.

### CROSS-REFERENCE TO RELATED APPLICATIONS

This application is a continuation of International Patent Application No. PCT/US2023/025791, filed in the U.S. Receiving Office on Jun. 20, 2023, which claims the benefit of U.S. Provisional Application 63/353,729, filed on Jun. 20, 2022, U.S. Provisional Application 63/444,523, filed on Feb. 9, 2023, U.S. Provisional Application 63/460,201, filed on Apr. 18, 2023, and U.S. Provisional Application 63/470,621, filed on Jun. 2, 2023. The entirety of each of these applications is hereby incorporated by reference for all purposes.

### FIELD OF THE INVENTION

This invention is in the area of the use of a specific heterocyclic based compound to selectively inhibit cyclin-dependent kinase 2 (CDK2) for the treatment of medical disorders characterized by aberrant cellular division, including but not limited to the treatment of tumors and cancers.

### BACKGROUND

Adult tissue is mainly comprised of terminally differentiated, quiescent cells that have exited the cell cycle. Physiological cues responding to extracellular stimuli including, for example, tissue injury, can trigger some cells to re-enter the cell cycle and divide to replenish damaged or dead cells (Matthews et al. Nat Rev Mol Cell Biol. 23:74-88(2022)). There is a well-regulated balance between cell division and programmed cell death (apoptosis).

Cell division is regulated by the cell cycle, which is divided into four phases: G1 phase (cell growth and machinery synthesis), S phase (DNA replication to generate two identical sets of chromosomes), G2 phase (cell growth and supplemental machinery synthesis), and M phase (single cell divides into two identical daughter cells). The progression between cell cycle phases is primarily governed by cyclins and cyclin-dependent kinases (CDKs) (Asghar et al. Nat Rev Drug Discov. 14(2): 130-46(2015)), which are activated or inhibited in response to a complex system of cell signaling networks that interpret extracellular signals.

Cyclins bind and activate CDKs which preferentially phosphorylate the tumor suppressor retinoblastoma (Rb) protein. The Rb protein, when dephosphorylated, binds and represses E2 factor (E2F) transcription factors, recruits co-repressors, and represses the transcription of cell cycle genes that are regulated by E2Fs. When Rb is phosphorylated by CDK/cyclin complexes, however, it dissociates from E2F transcription factors which then transcribe a suite of genes involved in cell cycle control including, for example, cyclin E (CCNE), cyclin A (CCNA), and cyclin B1 (CCNB1), and progresses the cell into a subsequent phase of the cell cycle (Hochegger et al. Nat Rev Mol Cell Biol. 9:910-16(2008)). Following cell division during the mitotic (M) phase, the cell shuts off expression of the machinery required for genetic replication and segregation and exits the cell cycle.

A range of neoplastic disorders develop when there occurs uncontrolled cellular proliferation. The development of broad-spectrum, anti-neoplastic agents have been leveraged to treat a variety of cellular proliferative disorders including ovarian, breast, and lung cancers (see, e.g., Jordan & Wilson, Nat Rev Canc. 4:253-65(2004)). Organometallic-based chemotherapeutic agents such as cisplatin have been used to treat cancers including lymphomas, sarcomas, germ cell tumors, and carcinomas such as small cell lung cancer (SCLC), bladder cancer, ovarian cancer.

One such organometallic agent, cisplatin, binds nitrogenous bases and induces profound DNA cross-linking that results in apoptosis (see, e.g., Siddick et al. Oncogene. 22:7265-79(2003)). Separately, agents which intercalate or alkylate DNA have been exhaustively tested in the clinic for the treatment of cancers. The toxicity associated with these drugs is a foremost concern for patients that require long-term therapy.

The dysregulation of cyclin dependent kinases and cyclins provide the impetus for excessive neoplasm growth and tumor development, eventually triggering cell division disorders such as cancer (Otto & Sicinski. Nat Rev Canc. 17:93-115(2017)). Recently, targeted approaches involving the use of compounds to inhibit CDKs have been used to treat cancer and limit adverse events associated with broad-spectrum anti-neoplastic agents (O'Leary et al. Nat Rev Clin Oncol. 13:417-30(2016)). For example, palbociclib (PD-033299; IBRANCE®) was discovered by Pfizer researchers to transiently inhibit cell cycle progression in CDK4/6-dependent cells (Roberts et al. JNCI. 104(6): 476-87(2012)). In February 2015, the FDA approved IBRANCE® (palbociclib) for the treatment of estrogen receptor-positive (ER+), human epidermal growth factor receptor 2-negative (HER2−) breast cancer in combination with letrozole. The structure of palbociclib is:

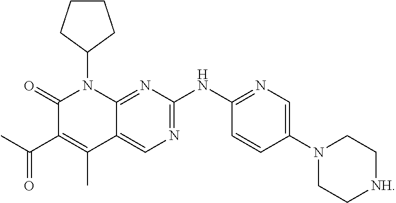 [SMILES: CC(=O)c1c(C)c2cnc(Nc3ccc(N4CCNCC4)cn3)nc2n(C2CCCC2)c1=O]

Ribociclib (Lee011; KISQALI®) is a CDK4/6 inhibitor approved by the FDA for use in combination with an aromatase inhibitor for the treatment of metastatic breast cancers. Ribociclib is currently being tested in clinical trials for the treatment of a variety of other cancers. The structure of ribociclib is:

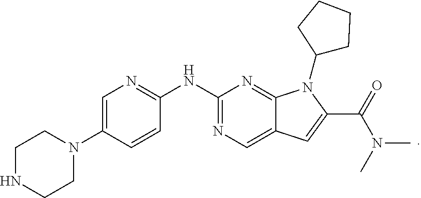 [SMILES: CN(C)C(=O)c1cc2cnc(Nc3ccc(N4CCNCC4)cn3)nc2n1C1CCCC1]

Abemaciclib (LY2835219; VERZENIO®) is a CDK4/6 inhibitor that is approved by the FDA for use in combination with endocrine therapy (tamoxifen or aromatase inhibitor) for adjuvant treatment of hormone receptor-positive (HR+), human epidermal growth factor receptor 2-negative (HER2−) breast cancers. The compound is also in a series of clinical trials including a phase III trial for the treatment of stage IV non-small cell lung carcinoma and another trial with either anastrozole or letrozole for the treatment of first line treatment of breast cancer. The structure of abemaciclib is:

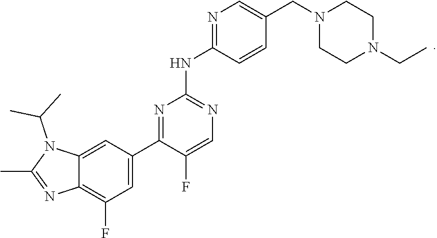 [SMILES: CCN1CCN(Cc2ccc(Nc3ncc(F)c(-c4cc(F)c5nc(C)n(C(C)C)c5c4)n3)nc2)CC1]

Despite demonstrated anti-tumor efficacy of the aforementioned compounds and generally favorable safety profile, there remain side effects associated with the treatment (Thill et al. Ther Adv Med Oncol. 10:1758835918793326 (2018)), with neutropenia the most commonly observed adverse effect. Novel CDK4/6 inhibitors have been demonstrated to preserve myeloid cells during cancer treatment and are expected to soon be approved by the FDA for the treatment of cancer.

Trilaciclib (COSELA™) is an FDA approved selective CDK4/6 inhibitor from G1 Therapeutics, Inc., for use as a first in-class myelopreservative therapy designed to improve quality of life and outcomes in extensive-stage small cell lung cancer (SCLC) patients receiving chemotherapy by preserving hematopoietic stem and progenitor cells (HSPCs). Trilaciclib is a short-acting CDK4/6 inhibitor administered intravenously prior to chemotherapy and is being examined in four randomized Phase II clinical trials, including as a first-line therapy in combination with carboplatin/etoposide chemotherapy regimen and the checkpoint inhibitor Tecentriq® (atezolizumab) for the treatment of SCLC. Trilaciclib has the structure:

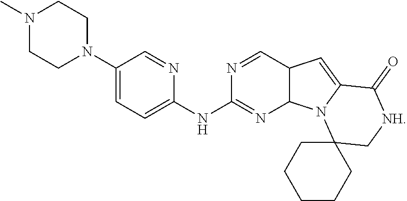 [SMILES: CN1CCN(c2ccc(NC3=NC4C(C=N3)C=C3C(=O)NCC5(CCCCC5)N34)nc2)CC1]

Lerociclib is a selective CDK4/6 inhibitor in clinical development by EQRx, Inc., the exclusive licensee of G1 Therapeutics, Inc., for use in combination with other anti-cancer agents for the treatment of multiple oncology indications. Lerociclib is in two ongoing Phase 1/2 clinical trials including a trial combining lerociclib with fulvestrant (Faslodex®) for patients with estrogen receptor-positive (ER+), HER2− breast cancer (NCT02983071) and a trial in combination with osmirtinib (Tagrisso®) for the treatment of epidermal growth factor receptor mutant (EGFRmut) non-small cell lung cancer. Lerociclib has the structure:

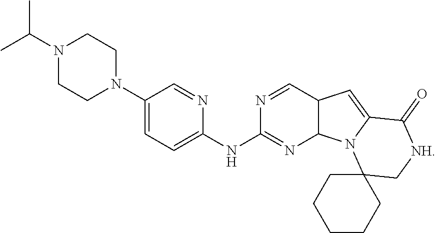 [SMILES: CC(C)N1CCN(c2ccc(NC3=NC4C(C=N3)C=C3C(=O)NCC5(CCCCC5)N34)nc2)CC1]

Additional pyrimidine-based anti-CDK2 agents are described in WO2021/236650 which is assigned to G1 Therapeutics, Inc. including the compound of structure:

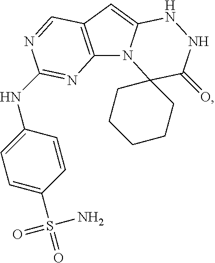 [SMILES: NS(=O)(=O)c1ccc(Nc2ncc3cc4n(c3n2)C2(CCCCC2)C(=O)NN4)cc1]

and its process of preparation.

Despite progress in the development of cell cycle inhibiting compounds to treat disorders of abnormal cellular proliferation in a subject, for example, a human, there remains an unmet need for new strategies to combat these lethal disorders.

It is an aim of the present invention to provide new uses and combinations that prevent uncontrolled cell cycling in a subject, for example, a human.

### SUMMARY

It has been discovered that the selective CDK2 inhibitor Compound I, or a pharmaceutically acceptable salt thereof, or a morphic form as described herein, is a potent therapeutic agent useful to treat abnormal proliferations that have increased cyclin E expression or cyclin E amplification. Compound I, or a pharmaceutically acceptable salt thereof, or a morphic form described herein, can also be used for the treatment of cancers that have developed resistance to CDK 4/6 inhibitors. Still further, Compound I, or a pharmaceutically acceptable salt thereof, or a morphic form described herein, can also be used for the treatment of cancers that have developed resistance to estrogen receptor degraders, for example, but not limited to selective estrogen receptor degraders (SERDs) such as fulvestrant or elacestrant. Compound I, or a pharmaceutically acceptable salt thereof, or a morphic form described herein, can also be used for the treatment of small cell lung cancer (SCLC), a retinoblastoma-null cancer for which there are no approved targeted therapies. As described further below, new and highly stable morphic forms and salts of Compound I have also been discovered.

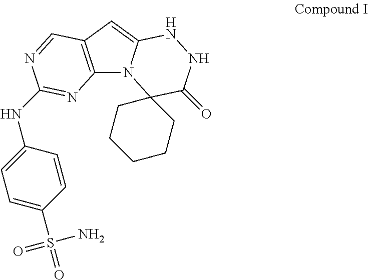 [SMILES: NS(=O)(=O)c1ccc(Nc2ncc3cc4n(c3n2)C2(CCCCC2)C(=O)NN4)cc1]

Compound I, or a pharmaceutically acceptable salt thereof, or a morphic form described herein, has several advantageous properties that can be used in the treatment of cyclin E overexpressed or amplified cancers, CDK4/6 inhibitor resistant cancers, estrogen receptor degrader resistant cancers, and/or small cell lung cancers (see, e.g., FIG. 151). Non-limiting examples of these advantageous properties include (i) selective inhibition of CDK2 (see, e.g., FIGS. 1A-1B and 2A-2C of Example 1); (ii) inhibition of CDK2 complexed with several cyclin binding partners (see, e.g., FIG. 1A-1B of Example 1); (iii) substantially long residence time when complexed with CDK2 (see, e.g., FIG. 1B of Example 1); (iv) a robust safety profile when administered to healthy, non-cancerous cells (see, e.g., FIGS. 3-4, 5A-5C, and 6 of Example 2); (v) synergy with chemotherapeutic agents for greater anti-tumor potency (see, e.g., FIG. 29A-29C, FIG. 30A-30C of Example 5); (vi) enhanced anti-tumor potency in CDK4/6 inhibitor-resistant cells when administered in combination with a CDK4/6 inhibitor (see, e.g., FIG. 7A-7D, 8A-8I, 9A-9B, 149 of Examples 3 and 45); (vii) anti-tumor potency in cells with amplified cyclin E activation and/or expression (see, e.g., FIG. 10A-10E, 11A-11B, 12, 13A-13C, 14A-14B, 16A-16B, 17A-17B, 18, 19A-19B, 20, and 21A-21B of Example 4); (viii) an induced change in expression and post-translational modification of cell cycle-related proteins (see, e.g., FIG. 7C, 8B, 10E, 11A-11B, 12, 20, 26A-26C, 146E-146F, 147L of Examples 4, 5, 6, and 45); (ix) induction of a targeted DNA-damage response in neoplastic cells (see, e.g., 22D-22E, 25, 27A-27D, and 28C of Example 5); (x) robust cell cycle arrest in naïve and therapy-resistant cells (see, e.g., FIG. 9A-9B, 10C-10D, 13A-13C, 14B, 15A-15D, 21B, 23, 24, 28B, 147D-147J of Examples 3-5 and 45) (xi) development of cellular senescence following cell cycle arrest in therapy-resistant cells (see, e.g., FIG. 148A-148F of Example 45) (xii) durable in vivo tumor inhibition and tumor stasis in subjects having a cellular proliferative disorder, for example, a cancer (see, e.g., FIG. 18, 19A, 31A-31G, 146A-146D, 149 of Examples 4, 6, 44, and 45); (xiii) improved overall survival as evidenced by animal studies in subjects having a cellular proliferative disorder, for example, a cancer (see, e.g., FIG. 19B of Example 4); (xiv) prolonging time to resistance to other anticancer agents, for example SERDs (see, e.g., FIG. 145, Example 43); (xv) little to no inhibition of human drug transporters suggesting low drug-drug interaction toxicity risks (see, e.g., FIG. 127-143 of Example 41); and/or (xvi) excellent tolerance of daily administration in multiple animal models (see, e.g., Examples 42 and 45).

In addition to its potent anti-cancer activity, Compound I or a pharmaceutically acceptable salt thereof or morphic form described herein can be used to resensitize relapsed and/or refractory cancers to other anti-cancer therapies including but not limited to CDK4/6 inhibitors, estrogen receptor degraders and inhibitors, chemotherapeutic agents, immune checkpoint inhibitors, or a combination thereof. This improvement provides a significant advance in the state of the art of cancer treatment. In certain embodiments, Compound I or a pharmaceutically acceptable salt thereof or morphic form described herein causes resensitization by favorably altering gene expression profiles in the cell resulting in a tumor microenvironment that is amenable to treatment with therapeutic agents that it was previously resistant to. The net result of this effect on the microenvironment of the tumor is an improvement in the subject's response to therapeutics, including in some non-limiting cases therapeutics that were previously administered to the subject and the subject subsequently became resistant to (i.e., re-sensitizing a tumor), to effectively combat the cancer or tumor, increasing the ability to achieve short term (up to approximately 1, 2, 3, 4, 5, or 6 months), long term (up to 7, 8, 9, 10, 11 or 12 months or greater) or complete responses.

As a result of the remarkable CDK2 inhibitor properties of Compound I, new advantageous treatments of disorders mediated by cyclin E overexpression/amplification, CDK4/6 inhibitor resistance, estrogen receptor degrader resistance, and/or Rb-independent mechanisms, for example small cell lung cancer, have been discovered. In non-limiting embodiments of the invention, Compound I or a pharmaceutically acceptable salt, morphic form, or pharmaceutical composition thereof can be used in the following non-limiting indications for example:
(a) use of Compound I, or a pharmaceutically acceptable salt thereof, or a morphic form described herein, for the treatment of a human having a cyclin E amplified or overexpressed abnormal cellular proliferative disorder, including but not limited to a cyclin E amplified or overexpressed ovarian cancer, gastric cancer, breast cancer, bladder cancer, lung cancer, or gynecological cancer;
(b) use of Compound I, or a pharmaceutically acceptable salt thereof, or morphic form described herein, for treating a subject with an abnormal cellular proliferative disorder, for example cancer, comprising obtaining a sample from a human, detecting whether cyclin E1 and/or E2 (CCNE1/CCNE2) are overexpressed in the sample compared with a control sample and, upon determining CCNE1 and/or CCNE2 are overexpressed, administering to the human an effective amount of a selective CDK2 inhibitor having the structure of Compound I, or a pharmaceutically acceptable salt thereof, or morphic form described herein;
(c) use of Compound I, or a pharmaceutically acceptable salt thereof of morphic form described herein, for the treatment of a human having a small cell lung cancer wherein the human is further administered an additional therapeutic agent in combination with Compound I, for example doxorubicin or camptothecin;
(d) use of Compound I, or a pharmaceutically acceptable salt thereof or morphic form described herein, in combination with a CDK4/6 inhibitor, for example lerociclib, palbociclib, abemaciclib, or ribociclib, for the treatment of a subject having an acquired CDK4/6 inhibitor-resistant cancer;
(e) use of Compound I, or a pharmaceutically acceptable salt thereof or morphic form described herein, in combination with a CDK4/6 inhibitor, for example lerociclib, palbociclib, abemaciclib, or ribociclib, for the treatment of a subject having an instrinsic CDK4/6 inhibitor-resistant cancer;
(f) use of Compound I, or a pharmaceutically acceptable salt thereof or morphic form described herein, in combination with an estrogen inhibitor such as an estrogen receptor degrader, for example fulvestrant or elacestrant, for the treatment of a subject having an estrogen inhibitor-resistant cancer;
(g) use of Compound I, or a pharmaceutically acceptable salt thereof or morphic form described herein, in combination with a CDK4/6 inhibitor, for example lerociclib, palbociclib, abemaciclib, or ribociclib, and an estrogen inhibitor, for example fulvestrant or elacestrant, for the treatment of a subject having a CDK4/6 inhibitor-resistant and/or estrogen inhibitor-resistant cancer;
(h) the use of (d-f) wherein the CDK4/6 inhibitor-resistant cancer is cyclin E amplified or overexpressed;
(i) the use of (d) or (f) wherein the CDK4/6 inhibitor-resistant cancer is estrogen receptor-positive (ER+) breast cancer;
(j) the use of any one of (a)-(h) wherein the abnormal cellular proliferative disorder is an unresectable cancer;
(k) the use of any one of (a)-(i) wherein the abnormal cellular proliferative disorder is an advanced cancer;
(l) the use of any one of (a)-(j) wherein the abnormal cellular proliferative disorder is solid tumor;
(m) the use of any one of (a)-(k) wherein the abnormal cellular proliferative disorder is an advanced cancer;
(n) the use of any one of (i)-(1) wherein the cancer is metastatic;
(o) the use of any one of (i)-(1) wherein the cancer is platinum-resistant or platinum-refractory;
(p) the use of any one of (i)-(1) wherein the cancer is CCNE1 amplified;
(q) a morphic form of Compound I as described herein;
(r) a pharmaceutical composition comprising a morphic form of Compound I and a pharmaceutically acceptable excipient;
(s) a pharmaceutical composition comprising Compound I and a pharmaceutically acceptable excipient wherein the pharmaceutical composition is prepared from a morphic form of Compound I, for example, a spray dry dispersion;
(t) the pharmaceutical composition of (q) or (r) wherein the pharmaceutically acceptable excipient is a diluent, for example polyethylene glycol;
(u) the use of any one of (a)-(o) wherein Compound I is a morphic form or a pharmaceutical composition of any one of (q)-(s); or
(v) use of a morphic form or pharmaceutical composition of any one of (q)-(s) in the treatment of a CDK2 mediated abnormal cellular proliferation for example a CDK2 mediated cancer.

In certain aspects, Compound I or a pharmaceutically acceptable salt thereof or morphic form described herein is used to treat a Rb-dependent cancer wherein the cancer has overexpressed or amplified cyclin-E expression. For example, in certain embodiments Compound I or a pharmaceutically acceptable salt thereof or morphic form described herein is used to treat a breast cancer, prostate cancer (including androgen-resistant prostate cancer), colon, including metastatic colon, another cancer of the reproductive system such as endometrial, ovarian or testicular cancer, which has overexpressed or amplified cyclin-E expression. Cancers with overexpressed or amplified cyclin-E expression may have an overexpression or amplification of CCNE1 and/or CCNE2. In certain embodiments the cycle-E amplified or overexpressed cancer has an amplification or overexpression of CCNE1. In certain embodiments the cycle-E amplified or overexpressed cancer has an amplification or overexpression of CCNE2.

In yet another embodiment, Compound I or a pharmaceutically acceptable salt thereof or morphic form described herein is administered in an effective amount for the treatment of abnormal tissue with overexpressed or amplified cyclin-E expression of the female reproductive system such as breast, ovarian, endometrial, or uterine cancer, in combination or alternation with an effective amount of an estrogen inhibitor including but not limited to a SERM (selective estrogen receptor modulator), a SERD (selective estrogen receptor degrader), a complete estrogen receptor degrader, or another form of partial or complete estrogen antagonist. In some embodiments, Compound I or a pharmaceutically acceptable salt thereof or morphic form described herein is administered to the subject at least once daily, and wherein an effective amount of the estrogen inhibitor is administered according to its prescribed label. In some embodiments, Compound I or a pharmaceutically acceptable salt thereof or morphic form described herein is administered to the subject at least twice daily, and wherein an effective amount of the estrogen inhibitor is administered according to its prescribed label.

In some embodiments, the cyclin E amplified or overexpressed abnormal cellular proliferative disorder is selected from ovarian cancer, uterine cancer, uterine carcinosarcoma (UCS), uterine corpus endometrial carcinoma (UCEC), ovarian cancer, ovarian serous cystadenocarcinoma (OV), sarcoma (SARC), lung cancer, lung squamous cell carcinoma (LUSC), lung adenocarcinoma (LUAD), stomach cancer, stomach adenocarcinoma (STAD), bladder cancer, bladder urothelial carcinoma (BLCA), esophageal cancer, esophageal carcinoma (ESCA), adrenocortical carcinoma, breast cancer, breast invasive carcinoma (BRCA), pancreatic cancer, pancreatic adenocarcinoma (PAAD), fallopian tube cancer, primary peritoneal cancer liver cancer, liver hepatocellular carcinoma (LIHC), cervical cancer, cervical squamous cell carcinoma (CESC), endocervical adenocarcinoma, mesothelioma (MESO), head and neck squamous cell carcinoma (HSNC), colon cancer, colon adenocarcinoma (COAD), skin cancer, melanoma, skin cutaneous melanoma (SKCM), glioblastoma multiforme (GBM), kidney cancer, or kidney chromophobe (KICH). In some embodiments, the cyclin E amplified or overexpressed cancer is an ovarian cancer. In some embodiments, the cyclin E amplified cancer is CDK4/6 inhibitor-resistant. In certain embodiments, the cancer is advanced and/or metastatic cancer. In certain embodiments, the cancer is advanced unresectable cancer. In certain embodiments, the cancer is platinum-refractory and/or platinum-resistant. In certain embodiments, the cancer has progressed following a prior standard of care regimen. In certain embodiments, the cancer has progressed following a prior standard systemic therapy. In certain embodiments, the cancer has progressed following a prior systemic anti-cancer therapy. In certain embodiments, the cancer has progressed following a prior regimen comprising a platinum analog. In certain embodiments, the cancer has progressed following a prior regimen comprising a CDK4/6 inhibitor. In some embodiments, the cyclin E amplified or overexpressed cancer is uterine cancer. In some embodiments, the cyclin E amplified or overexpressed cancer is ovarian cancer. In some embodiments, the cyclin E amplified or overexpressed cancer is breast cancer. In some embodiments, the cyclin E amplified or overexpressed cancer is prostate cancer. In some embodiments, the cyclin E amplified or overexpressed cancer is bladder cancer. In some embodiments, the cyclin E amplified or overexpressed cancer is a sarcoma.

In certain embodiments, the method described above further comprise administering an effective amount of one or more an additional anti-cancer therapies. In some embodiments, the anti-cancer therapy is selected from radiation, surgery, an immune checkpoint inhibitor, an estrogen inhibitor, an androgen inhibitor, a PARP inhibitor, or a combination thereof. In certain embodiments, the method further comprises administering an effective amount of an estrogen inhibitor. In some embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In some embodiments, the estrogen inhibitor is a selective estrogen receptor degrader (SERD). In some embodiments, the SERD comprises fulvestrant. In some embodiments, the SERD comprises elacestrant (RAD1901). In some embodiments, Compound I or a pharmaceutically acceptable salt thereof or morphic form described herein is administered to the subject at least once daily, and wherein an effective amount of the anti-cancer therapy is administered according to its prescribed label. In some embodiments, Compound I or a pharmaceutically acceptable salt thereof or morphic form described herein is administered to the subject at least twice daily, and wherein an effective amount of the anti-cancer therapy is administered according to its prescribed label.

In other aspects Compound I or a pharmaceutically acceptable salt thereof or morphic form described herein is used to treat small cell lung cancer (an Rb-null cancer). In certain embodiments Compound I inhibits the CDK2/A axis leading to inhibition of small cell lung cancer growth or remission. In other embodiments Compound I causes DNA damage in small cell lung cancer cells arresting growth or leading to remission.

For example, in certain embodiments a use is provided for treating a subject with a CDK4/6 inhibitor-resistant small cell lung cancer (SCLC) comprising (i) administering to the subject an effective amount of Compound I, or a pharmaceutically acceptable salt thereof or morphic form described herein; and (ii) administering to the subject an effective amount of a chemotherapeutic agent, wherein Compound I or a pharmaceutically acceptable salt thereof or morphic form described herein is administered to the subject within 24 hours or less prior to or concomitantly with the administration of the chemotherapeutic agent. In some embodiments, the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof. In some embodiments, the chemotherapeutic agent is doxorubicin. In some embodiments, the chemotherapeutic agent is camptothecin. In some embodiments, the chemotherapeutic agent is cisplatin. In some embodiments, Compound I or a pharmaceutically acceptable salt thereof or morphic form described herein is administered to the subject within 6 hours or less to the administration of the chemotherapeutic agent. In some embodiments, Compound I or a pharmaceutically acceptable salt thereof or morphic form described herein is administered to the subject within 3 hours or less to the administration of the chemotherapeutic agent. In some embodiments, Compound I or a pharmaceutically acceptable salt thereof or morphic form described herein is administered to the subject at least once daily, and wherein an effective amount of the chemotherapeutic agent is administered according to its prescribed label or a standard of care. In some embodiments, Compound I or a pharmaceutically acceptable salt thereof or morphic form described herein is administered to the subject at least twice daily, and wherein an effective amount of the chemotherapeutic agent is administered according to its prescribed label or standard of care.

In another aspect, disclosed herein is a method for treating a subject with a CDK4/6 inhibitor-resistant cancer comprising administering to the subject an effective amount of Compound I, or a pharmaceutically acceptable salt thereof or morphic form described herein; and, administering to the subject an effective amount of a CDK4/6 inhibitor. In certain embodiments, the CDK4/6 inhibitor resistant cancer has acquired resistance to a CDK4/6 inhibitor. In certain embodiments, the CDK4/6-inhibitor resistant cancer has progressed following a prior regimen comprising a CDK4/6 inhibitor. In some embodiments, the CDK4/6 inhibitor of the prior regimen is selected from palbociclib, ribociclib, abemaciclib, trilaciclib, lerociclib, SHR6390 (dalpiciclib), or a combination thereof. In some embodiments, the CDK4/6 inhibitor of the prior regimen is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In some embodiments, the CDK4/6 inhibitor-resistant cancer is retinoblastoma (Rb) protein-positive (Rb+). In some embodiments, the CDK4/6-inhibitor resistance cancer has an intrinsic CDK4/6 inhibitor resistance. In some embodiments, the CDK4/6 inhibitor cancer is retinoblastoma (Rb) protein-null (Rb−). In some embodiments, the CDK4/6-inhibitor resistant cancer is selected from breast cancer, lung cancer, uterine cancer, endometrial cancer, ovarian cancer, prostate cancer, bladder cancer, testicular cancer, glioblastoma, head and/or neck cancer, or prostate cancer. In some embodiments, the CDK4/6-inhibitor resistant cancer is breast cancer. In some embodiments, the CDK4/6-inhibitor resistant breast cancer is estrogen receptor-positive (ER+) breast cancer. In certain embodiments, the CDK 4/6-inhibitor resistant cancer is hormone receptor positive (HR+) breast cancer. In certain embodiments, the CDK4/6-inhibitor resistant cancer is cyclin E amplified or overexpressed. In some embodiments, Compound I or a pharmaceutically acceptable salt thereof or morphic form described herein is administered to the subject at least once daily, and wherein an effective amount of the CDK4/6 inhibitor is administered according to its prescribed label. In some embodiments, Compound I or a pharmaceutically acceptable salt thereof is administered to the subject at least twice daily, and wherein an effective amount of the CDK4/6 inhibitor is administered according to its prescribed label. In some embodiments, the CDK4/6 inhibitor resistant cancer is also resistant to endocrine therapy or estrogen inhibitor, for example a SERD.

In another aspect, disclosed herein is a method for treating a subject with a CDK4/6 inhibitor-resistant estrogen receptor-positive (ER+) breast cancer comprising administering to the subject an effective amount of Compound I, or a pharmaceutically acceptable salt thereof or morphic form described herein; and, administering to the subject an effective amount of a CDK4/6 inhibitor. In some embodiments, the CDK4/6 inhibitor-resistant ER+ breast cancer is tyrosine kinase-type cell surface receptor HER2-negative. In some embodiments, Compound I or a pharmaceutically acceptable salt thereof or morphic form described herein is administered to the subject at least once daily, and wherein an effective amount of the CDK4/6 inhibitor is administered according to its prescribed label or standard of care. In some embodiments, Compound I or a pharmaceutically acceptable salt thereof or morphic form described herein is administered to the subject at least twice daily, and wherein an effective amount of the CDK4/6 inhibitor is administered according to its prescribed label or standard of care.

In certain embodiments, the treatment results in a reduction of incidents of treatment-emergent adverse events in comparison to the predicted number of incidents of treatment-emergent adverse events in subjects receiving treatment without Compound I. In certain embodiments, the treatment results in a reduction of incidents of laboratory abnormalities in comparison to the predicted number of incidents of laboratory abnormalities in subjects receiving treatment without Compound I. In certain embodiments, the treatment results in an improved overall survival (OS) compared to subjects receiving treatment without Compound I. In certain embodiments, the treatment results in an improved overall response rate (ORR) compared to subjects receiving treatment without Compound I. In certain embodiments, the treatment results in an improved disease control rate (DCR) compared to subjects receiving treatment without Compound I. In certain embodiments, the treatment results in an improved progression free survival (PFS). In certain embodiments, the treatment results in an improved duration of response (DOR) compared to subjects receiving treatment without Compound I. In certain embodiments, the treatment results in an extension of time to progression (TTP) compared to subjects receiving treatment without Compound I.

In other aspects, an advantageous morphic form of Compound I is described with improved solubility, stability, crystallinity, flowability, and/or purity. Over two-dozen morphic forms of Compound I and various salts of Compound I were discovered and tested in an effort that identified eight advantageous salts in a variety of solvent conditions and temperatures. Salts of Compound I were made with counterions selected from sulfuric acid, methanesulfonic acid, maleic acid, phosphoric acid, L-(+)-tartaric acid, citric acid, hydrobromic acid, and benzenesulfonic acid. Several of these salts have multiple morphic forms which are described herein.

Compound I Free Base Pattern 1 is a highly crystalline and stable morphic form which can be used in the treatment of a CDK2 mediated disorder or in the manufacture of an amorphous form of Compound I (for example by spray dry dispersion) for the treatment of a CDK2 mediated disorder.

Compound I HCl Pattern 1 is a particularly stable crystalline morphic form of Compound I distinguished from other salts by high purity and improved 7-day indicative stability at 40° C./75% RH, 7-day form stability at 80° C. with only moderate hygroscopicity. Compound I HCl Pattern 1 can be used in the treatment of a CDK2 mediated disorder or in the manufacture of an amorphous form of Compound I or a pharmaceutically acceptable salt thereof (for example by spray dry dispersion) for the treatment of a CDK2 mediated disorder.

In certain aspects, a pharmaceutical composition is provided comprising Compound I or a pharmaceutically acceptable salt thereof or a morphic form of Compound I or a pharmaceutically acceptable salt thereof and one or more pharmaceutically acceptable excipients is provided. In certain embodiments, the pharmaceutical composition includes polyethylene glycol (PEG). In certain embodiments, the pharmaceutical composition includes hydroxypropylmethylcellulose.

In other aspects, a pharmaceutical composition comprising amorphous Compound I or a pharmaceutically acceptable salt thereof and one or more pharmaceutically acceptable excipients is provided, wherein the pharmaceutical composition is manufactured from a morphic form of Compound I or a pharmaceutically acceptable salt thereof described herein. In certain embodiments, the manufactured pharmaceutical composition includes polyethylene glycol (PEG). In certain embodiments the manufactured pharmaceutical composition includes hydroxypropylmethylcellulose.

### BRIEF DESCRIPTION OF THE DRAWINGS

As referenced in the figures, PF-07104091 is

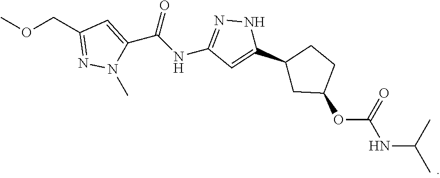 [SMILES: COCc1cc(C(=O)Nc2cc([C@H]3CC[C@@H](OC(=O)NC(C)C)C3)[nH]n2)n(C)n1]

FIG. 1A-B is a collection of tables showing that Compound I CDK2/E selectively inhibits CDK2/E to a degree that exceeds Pfizer CDK2 inhibitor compound PF-07104091. Experimental conditions for these experiments are found in Example 1. FIG. 1A is a table illustrating biochemical profiles of novel and potent CDK2 inhibitors against a panel of CDKs and respective binding partners. Assays were completed in a 12-point dose-response format. Results are shown as nanomolar IC₅₀ concentrations against each target. The Pfizer selective CDK2 inhibitor, PF-07104091, was included as a reference compound. ND, not determined. Compound was shown to preferentially bind to CDK2/E over CDK1/B and CDK9/T. Furthermore, Compound I is 4-fold more potent against CDK2/E compared to PF-07104091. FIG. 1B is a table illustrating Compound I residence time, the lifetime of compound-target interaction, with several CDK/cyclin complex partners in a TR-FRET binding assay. Compound I complexes with CDK2/E and CDK2/A longer than CDK1/B and CDK9/T.

FIG. 2A-C shows that Compound I demonstrates robust intracellular selectivity towards CDK2/E and limited CDK 1/B or CDK9/T activity. Experimental conditions for these experiments are found in Example 1. FIG. 2A is a grouped line graph illustrating normalized percent bioluminescence resonance energy transfer (NanoBRET) response on the y-axis and Compound I concentration as log (M) on the x-axis. NanoBRET Target Engagement Intracellular Kinase Assay results demonstrates potent inhibition of CDK2/E (circle) but not CDK1/B1 (square) or CDK9/T1 (triangle) by Compound I. The experiment was performed in HEK-293 cells transiently expressing CDK2-Nluc, CDK1-Nluc, or CDK9-Nluc fusion. FIG. 2B is a table showing Compound I intracellular inhibition IC₅₀ values towards CDK2/E, CDK1/B, and CDK9/T. The IC₅₀ values are also represented as fold-changes to the IC₅₀ value observed for Compound I towards CDK2/E. FIG. 2C is a table showing Compound I NanoBRET Target Engagement Intracellular Kinase Assay IC₅₀ values towards CDK2/E, CDK1/B, CDK2/A, CDK4/D1, CDK6/D3, and CDK9/T. The IC₅₀ values are also represented as fold-changes to the IC₅₀ value observed for Compound I towards CDK2/E. IC₅₀, half maximal inhibitor concentration.

FIG. 3 is a bar graph that shows that Compound I demonstrates limited cytotoxicity in normal H68 human fibroblast cells. Experimental conditions for these experiments are found in Example 2. The cellular proliferation IC₅₀ value for each treatment of Hs68 cells, which are normal human fibroblasts, is shown on the y-axis for different CDK inhibitors including dinaciclib, palbociclib, and Compound I on the x-axis. Limited cytotoxic activity was observed for Compound I in Hs68 cells. IC₅₀, half maximal inhibitor concentration.

FIG. 4 shows that the administration of Compound I alone or in combination with cisplatin induces little cell cycle alterations in normal H68 human fibroblast cells. Experimental conditions for these experiments are found in Example 2. FIG. 4 is a bar graph showing the percent of Hs68 cells represented on the y-axis measured at different cell cycle states, including DNA content <2N, G0/G1 phase, S phase, or G2/M phase, for different treatment conditions represented on the x-axis.

FIG. 5A-C shows that Compound I does not induce caspase 3/7 activation over an extended course of administration to normal human fibroblast Hs68 cells. Experimental methods for these experiments are found in Example 2. FIG. 5A is a grouped line plot showing cell ratio to control values on the y-axis for cells treated with staurosporine (circle), Compound I (square), and PF-07104091 (triangle) for 24 hours at different molar concentrations on the x-axis. FIG. 5B is a grouped line plot showing cell ratio to control values on the y-axis for cells treated with staurosporine (circle), Compound I (square), and PF-07104091 (triangle) for 48 hours at different molar concentrations on the x-axis. FIG. 5C is a grouped line plot showing cell ratio to control values on the y-axis for cells treated with staurosporine (circle), Compound I (square), and PF-07104091 (triangle) for 72 hours at different molar concentrations on the x-axis.

FIG. 6 shows that Compound I either alone or in combination with cisplatin does not induce γ-H2AX formation in normal Hs68 human fibroblast cells. Experimental conditions for these experiments are found in Example 2. FIG. 6 is a bar graph showing the percent of cells positive for γ-H2AX marker on the y-axis for different treatment conditions comprising cisplatin and/or Compound I administered to Hs68 fibroblast cells. The symbol “>” indicates a sequential, staggered co-administration of the molecules indicated. The symbol “+” indicates two molecules were simultaneously administered.

FIG. 7A-D shows the generation and validation of palbociclib-resistant breast cancer model MCF7 cells. Experimental methods for these experiments are found in Example 3. FIG. 7A shows pictures of cultured cells depicting similar morphology and growth of breast cancer model MCF7 parental cells (MCF7 Parental), and cells cultured in the presence of palbociclib for four months (MCF7 Palbo-R). The MCF7 cell line is a well-characterized model system of estrogen receptor-positive (ER+) breast cancer. FIG. 7B is a bar graph showing Log2-fold changes on the x-axis of genes of interest indicated on the y-axis (*p-value<0.05). MCF7 Palbo-R cells exhibited upregulation of CDK6, CCNE1, and CCNE2 transcripts, markers of acquired CDK4/6 inhibitor resistance. FIG. 7C is a western blot for retinoblastoma protein (Rb), Cyclin E, and input loading control GAPDH in palbociclib-sensitive (Palbo-S) MCF7 parental control cells (left) and MCF7 Palbo-R daughter cells. FIG. 7D is a bar graph analyses showing quantified immunoblot signal illustrated on the y-axis of Rb and Cyclin E proteins displayed on the x-axis normalized to GAPDH loading control in MCF7 Palbo-S parental (open square) and MCF7 Palbo-R daughter (black square) cell lines. Cyclin E levels are increased in MCF7 Palbo-R cells.

FIG. 8A-I shows that palbociclib-resistant (Palbo-R) breast cancer model MCF7 cells are highly sensitive to a combination treatment comprising the administration of both Compound I and palbociclib. Experimental methods for these experiments are found in Example 3. FIG. 8A is a bar graph showing cellular proliferation IC₅₀ values in nanomolar (nM) on the y-axis and different treatment regimens on the x-axis. MCF7 Palbo-R cells are exquisitely sensitive to combined CDK2 and CDK4/6 inhibition comprising the administration of both Compound I and palbociclib compared with either agent alone. FIG. 8B is a western blot showing levels of phosphorylated residues 807 and 811 of retinoblastoma protein (pRb 807/811) and beta-tubulin as an input loading control under no PD-0332991 (palbocilib) or 1 μM of palbociclib administered in combination with increasing concentrations of Compound I. Compound I potently inhibited Rb phosphorylation in MCF7 Palbo-R cells when combined with PD-0332991 (palbocilib). IC₅₀, half maximal inhibitor concentration. FIG. 8C is a line graph showing ratio of viable MCF7 PDRCL5-1 no PD cells to control on the y-axis and Compound I concentrations in molarity on the x-axis. MCF7PDRC15-1 cells were grown in presence of luM palbociclib for maintenance. FIG. 8D is a line graph showing ratio of viable MCF7 PDRCL5-1 no PD cells to control on the y-axis and Compound I concentrations in molarity on the x-axis. FIG. 8F-I are bar graphs illustrating average colony area and percent size for different treatment conditions including: control (black bar); 300 nM palbociclib (white bar); palbociclib in combination with 300 nM Compound I (vertical line bar graph); 300 nM Compound I (diagonal line bar graph); and palbociclib followed by palbociclib in combination with Compound I (checkered bar graph). FIG. 8E are images culture plates containing T47D human breast carcinoma cell colonies under different treatment conditions at Week 2, 3, 5, or 8. FIG. 8F is a bar graph illustrating average colony size for different treatment conditions on the y-axis compared to control at Week 2, 3, 5, or 8 indicated on the x-axis. FIG. 8G is a bar graph illustrating percent colony area for different treatment conditions on the y-axis compared to control at Week 2, 3, 5, or 8 indicated on the x-axis. FIG. 8H is a bar graph illustrating average colony size for different treatment conditions on the y-axis at Week 2, 3, 5, or 8 indicated on the x-axis. FIG. 8I is a bar graph illustrating percent colony area for different treatment conditions on the y-axis at Week 2, 3, 5, or 8 indicated on the x-axis. The symbol “>” indicates a sequential, staggered co-administration of the molecules indicated. The symbol “+” indicates two molecules were simultaneously administered.

FIG. 9A-B shows that Compound I potently arrests palbociclib-resistant (Palbo-R) breast cancer model MCF7 cells in G0/G1 phase. Experimental methods for these experiments are found in Example 3. FIG. 9A is a bar graph illustrating the percent of MCF7 parental cells in different cell phases on the y-axis for different concentrations of Compound I illustrated on the x-axis. FIG. 9B is a bar graph illustrating the percent of Palbo-R MCF7 daughter cells in different cell phases on the y-axis for different concentrations of Compound I illustrated on the x-axis.

FIG. 10A-E shows that Compound I preferentially inhibits several independent CCNE^{high} expressing ovarian cancer model cell lines. Experimental methods for these experiments are found in Example 4. FIG. 10A is a bar graph showing the percent of tumors exhibiting cyclin E1 expression and/or activation increases compared to control on the y-axis for different cancer types as indicated on the x-axis. UCS: uterine carcinosarcoma; OV: ovarian serous cystadenocarcinoma; SARC: sarcoma; LUSC: lung squamous cell carcinoma; STAD: stomach adenocarcinoma; UCEC: uterine corpus endometrial carcinoma; BLCA: bladder urothelial carcinoma; LUAD: lung adenocarcinoma; ESCA: esophageal carcinoma; ACC: adrenocortical carcinoma; BRCA: breast invasive carcinoma; PAAD: pancreatic adenocarcinoma; LIHC: liver hepatocellular carcinoma; CESC: cervical squamous cell carcinoma and endocervical adenocarcinoma; MESO: mesothelioma; HNSC: head and neck squamous cell carcinoma; COAD: colon adenocarcinoma; SKCM: skin cutaneous melanoma; GBM: glioblastoma multiforme; KICH: kidney chromophobe. FIG. 10B is a bar graph showing cellular proliferation ICso values on the y-axis for different ovarian model cancer cell lines indicated on the x-axis. The cyclin E1 (CCNE1) status (amplified, gained, unamplified) for each cell line is indicated above the graph. FIG. 10C is a bar graph that demonstrates the percent of ovarian OVCAR-3 cells in G0-G1 phase, S phase, or G2-M phase on the y-axis at different concentrations of Compound I on the x-axis. FIG. 10D is a bar graph that demonstrates the percent of ovarian COV318 cells in G0-G1 phase, S phase, or G2-M phase on the y-axis at different concentrations of Compound I on the x-axis. FIG. 10E is a western blot image illustrating levels of phosphorylated threonine residue 821 of the retinoblastoma protein (pRb T821) of OVCAR-3 or COV318 cells administered vehicle (C) or increasing concentrations (30 nM, 100 nM, 300 nM, 10 mM) of Compound I. The blot is further labeled for CDK2 and β-tubulin as loading controls.

FIG. 11A-B shows that Compound I decreases phosphorylated retinoblastoma (pRb) in ovarian OVCAR3 cells in a dose- and temporal-dependent manner. Experimental methods for these experiments are found in Example 4. FIG. 11A is a western blot showing levels of cell cycle inhibitor proteins (p130, p27, p16), retinoblastoma protein (Rb), markers of phosphorylated Rb protein (pRb 807/811, pRb T821), cyclin-dependent kinase 2 (CDK2), cyclin proteins (CCNE1, CCNE2, CCNA2) in OVCAR-3 cells administered vehicle (C) or increasing concentrations (30 nM, 100 nM, 300 nM, 10 mM) of Compound I. β-actin is included as an input loading control. FIG. 11B is a western blot showing levels of pRb at residues 807 and 811 and cell cycle inhibitor protein p27 under increasing lengths of incubation of OVCAR-3 cells with Compound I. β-actin is included as an input loading control.

FIG. 12A-B shows that Compound I decreases phosphorylated retinoblastoma (pRb) in ovarian cancer model cells in a dose-dependent manner. Experimental conditions for these experiments are found in Example 4. FIG. 12A shows that Compound I decreases phosphorylated retinoblastoma (pRb) in a dose-dependent manner in ovarian cancer model FUOV1 and Kuramochi cells. Experimental conditions for these experiments are found in Example 4. FIG. 12A is a western blot showing levels of cell cycle inhibitor proteins (p130, p27, p16), retinoblastoma protein (Rb), markers of phosphorylated Rb protein (pRb 807/811, pRb T821, pRb S780), cyclin-dependent kinase 2 (CDK2), cyclin proteins (CCNE1, CCNE2, CCNA2) in FUOV1 cells administered vehicle (C) or increasing concentrations (30 nM, 100 nM, 300 nM, 1000 nM) of Compound I. FIG. 12B is a western blot showing levels retinoblastoma protein (Rb), markers of phosphorylated Rb protein (pRb 807/811, pRb T821), and cyclin A2 (CCNA2) in Kuramochi cells administered vehicle (0) or increasing concentrations (30 nM, 100 nM, 300 nM, 1000 nM) of Compound I.

FIG. 13A-C shows the cell cycle changes observed over time in ovarian cancer model OVCAR-3 cells treated with Compound I or the reference CDK2 inhibitor Pfizer compound PF-07104091. Experimental methods for these experiments are found in Example 4. FIG. 13A is a composite bar graph showing the percent of OVCAR-3 cells represented on the x-axis measured at different cell cycle states, including G1 phase, S phase, or G2/M phase, for different treatment conditions represented on the right-side y-axis for different lengths of time (24 hours or 48 hours of treatment) illustrated on the left-side y-axis. FIG. 13B is a composite bar graph showing the percent of OVCAR-3 cells represented on the bottom x-axis measured at different cell cycle states, including G1 phase, S phase, or G2/M phase, for different concentrations (100 nM, 300 nM, or 600 nM) listed on the top x-axis for different treatment conditions represented on the right-side y-axis for different lengths of time (24 hours or 48 hours of treatment) illustrated on the left-side y-axis. FIG. 13C is a composite bar graph showing the percent of OVCAR-3 cells represented on the x-axis measured at different cell cycle states, including G1 phase, S phase, or G2/M phase, for different treatment conditions represented on the right-side y-axis for different lengths of time (24 hours or 48 hours of treatment) illustrated on the left-side y-axis.

FIG. 14A-B shows the cell cycle changes observed in ovarian cancer model OVCAR-3 cells after increasing lengths of time following a washout of Compound I. Experimental conditions for these experiments are found in Example 4. FIG. 14A is a flow diagram showing the sequence of the experiment conducted. FIG. 14B is a bar graph showing the percent of OVCAR-3 cells represented on the y-axis measured at different cell cycle states, including DNA content <2N (black bar), G0/G1 phase (white bar), S phase (dark dotted bar), or G2/M phase (light dotted bar), for different treatment conditions represented on the x-axis. Compound I was administered at 300 nM for 0, 6, 18, 24, 30 or 48 hours.

FIG. 15A-15D shows APC histograms by EdU gating between 1 hour and 48 hours of treatment. Treatment conditions include DMSO control, 300 nM Compound I, or 300 nM PF-07104091. Experimental methods for these experiments are found in Example 4. FIG. 15A is a histogram showing cell count on the y-axis under different cell cycle stages, including G1, S, and G2 phase, indicated by stain signal magnitude illustrated on the x-axis. Graphs indicate a 1h to 48 h EdU trace. FIG. 15B is a histogram showing cell count on the y-axis under different cell cycle stages, including G1, S, and G2 phase, indicated by stain signal magnitude illustrated on the x-axis. Graphs indicate a 6h to 48 h EdU trace. FIG. 15C is a histogram showing cell count on the y-axis under different cell cycle stages, including G1, S, and G2 phase, indicated by stain signal magnitude illustrated on the x-axis. Graphs indicate a 24h to 48 h EdU trace. FIG. 15D is a histogram showing cell count on the y-axis under different cell cycle stages, including G1, S, and G2 phase, indicated by stain signal magnitude illustrated on the x-axis. Graphs indicate data collected at 48 h (no tracing).

FIG. 16A-B shows that Compound I is more cytotoxic than reference CDK2 inhibitor Pfizer compound PF-07104091 in CCNE^{high} ovarian cancer model Kuramochi and FUOV1 cells. Experimental methods for these experiments are found in Example 4. FIG. 16A is a grouped line plot showing cell ratio to control values on the y-axis over different concentrations of Compound I (circle) and palbociclib (square) on the x-axis in ovarian cancer cell model Kuramochi cells. FIG. 16B is a grouped line plot showing cell ratio to control values on the y-axis over different concentrations of Compound I (circle) and reference CDK2 inhibitor Pfizer compound PF-07104091 (square) on the x-axis in ovarian cancer cell model FUOV1 cells. The cellular proliferation IC₅₀ values in molar (M) units for Compound I and PF-07104091 are listed below the graph.

FIG. 17A-B shows that Compound I more potently inhibits CCNE^{high} ovarian cancer model OVCAR-3 cells than reference CDK2 inhibitor Pfizer compound PF-07104091. Experimental conditions for these experiments are found in Example 4. FIG. 17A is a grouped line graph illustrating OVCAR-3 cell number ratio to control on the y-axis and either Compound I (square) or PF-07104091 (triangle) molar concentration on the x-axis. FIG. 17B is a table listing the OVCAR-3 cellular proliferation IC₅₀ values for Compound I and PF-07104091 in nanomolar (nM) units.

FIG. 18 shows that Compound I is effective as a single agent in reducing tumor size. Tumor size (mm³) is represented on the y-axis over the course of administration carried out to 28 days illustrated on the x-axis in a CCNE^{high} ovarian OVCAR-3 tumor xenograft murine model. Experimental conditions for these experiments are found in Example 4. OVCAR-3 xenograft tumor fragments were harvested from host animals and implanted into immune-deficient mice (CR1:NU(NCr)-Fox1^{nu}). QD, Once Daily; BID, Twice Daily; mpk, milligrams per kilogram.

FIG. 19A-B shows an in vivo comparison of tumor potency and survival benefit for Compound I versus reference CDK2 inhibitor Pfizer compound PF-07104091 in a CCNE^{high} ovarian OVCAR-3 tumor xenograft murine model. OVCAR-3 xenograft tumor fragments were harvested from host animals and implanted into immune-deficient mice (CR1:NU(NCr)-Fox1^{nu}). Experimental conditions for these experiments are found in Example 4. FIG. 19A is a grouped line graph showing tumor volume (mm³) on the y-axis and day of treatment illustrated on the x-axis. Treatment conditions include: vehicle (circle); Compound I 100 mpk BID (open square); and PF-07104091 100 mpk BID (triangle). FIG. 19B is a survival line plot showing percent of living mice at the day of treatment illustrated on the x-axis. Treatment conditions include: vehicle (dotted line); Compound I 100 mpk BID (solid line); and PF-07104091 100 mpk BID (checkered line).

FIG. 20 shows that Compound I decreases phosphorylated retinoblastoma (pRb) in gastric cancer model MKN1 cells in a dose-dependent manner. Experimental conditions for these experiments are found in Example 4. FIG. 20 is a western blot showing levels of cell cycle inhibitor proteins (p130, p27, p21, p16), retinoblastoma protein (Rb), markers of phosphorylated Rb protein (pRb 807/811, pRb T821), cyclin-dependent kinase 2 (CDK2), cyclin proteins (CCNE1, CCNE2, CCNA2) in MKN1 cells administered vehicle (C) or increasing concentrations (30 nM, 100 nM, 300 nM, 10 mM) of Compound I. β-actin is included as an input-loading control.

FIG. 21A-B shows that cell cycle changes are observed in gastric cancer model MKN1 cells after increasing lengths of time following a washout of Compound I. Experimental conditions for these experiments are found in Example 4. FIG. 21A is a flow diagram showing the sequence of the experiment conducted. FIG. 21B is a bar graph showing the percent of MKN1 cells represented on the y-axis measured at different cell cycle states, including DNA content <2N (black bar), G0/G1 phase (white bar), S phase (dark dotted bar), or G2/M phase (light dotted bar), for different treatment conditions represented on the x-axis. Compound I was administered at 300 nM for 0, 6, 18, 24, 30, or 48 hours.

FIG. 22A-E shows that Compound I potently inhibits several independent small cell lung cancer (SCLC) model cell lines. Experimental methods for these experiments are found in Example 5. FIG. 22A is a bar graph that demonstrates the cellular proliferation IC₅₀ values of palbociclib or Compound I administered to SCLC model H526, SHP77, NCIH82, and NCIH69 cells indicated on the x-axis. FIG. 22B is a grouped line graph illustrating H526 cell number ratio to control on the y-axis and either Compound I (square) or PF-07104091 (triangle) molar (M) treatment concentration on the x-axis. FIG. 22C is a table listing the H526 cellular proliferation IC₅₀ values for Compound I and PF-07104091 in nanomolar (nM) units. FIG. 22D is a is a grouped line graph illustrating Caspase 3/7 activation ratio to control in H526 cells on the y-axis and staurosporine (circle), Compound I (square) or PF-07104091 (triangle) molar (M) concentration at 72 hours on the x-axis. Listed below the graph are the caspase 3/7 activation IC₅₀ values for each compound administered. FIG. 22E is a table listing the caspase 3/7 activation IC₅₀ concentration values for Compound I and PF-07104091 in nanomolar (nM).

FIG. 23 shows cell cycle changes exhibited by H526 cells undergoing nocodazole synchronization and release in the presence or lack thereof Compound I. Experimental conditions for these experiments are found in Example 5. FIG. 23 is a bar graph showing the percent of H526 cells represented on the y-axis measured at different cell cycle states, including DNA content <2N (black bar), G0/G1 phase (white bar), S phase (dark dotted bar), or G2/M phase (light dotted bar), for different treatment conditions represented on the x-axis. Compound I was administered at 300 nM for 6, 12, 24, 30 or 48 hours.

FIG. 24 shows H526 cell cycle changes over time following incubation with Compound I or PF-07104091. Experimental conditions for these experiments are found in Example 5. FIG. 24 is a composite bar graph showing the percent of H526 cells represented on the x-axis measured at different cell cycle states, including G1 phase, S phase, G2, or M phase, for different treatment conditions represented on the right-side y-axis for different lengths of time (1, 6, 24, or 48 hours of treatment) illustrated on the left-side y-axis.

FIG. 25 shows that Compound I induces DNA damage in small cell lung cancer (SCLC) model H526 cells and DNA damage is even greater when Compound I is administered in combination with cisplatin. Experimental conditions for these experiments are found in Example 5. FIG. 25 is a bar graph showing percent of cells positively immunolabeled with an antibody anti-γ-H2AX on the y-axis for different treatment conditions listed on the x-axis. The symbol “>” indicates a sequential, staggered co-administration of the molecules indicated. The symbol “+” indicates two molecules were simultaneously administered.

FIG. 26A-C shows that Compound I increases Cyclin E (CCNE1/CCNE2) levels and triggers increases in DNA damage markers in a dose-dependent manner in small cell lung cancer (SCLC) model H69 and H526 cells. Experimental methods for these experiments are found in Example 5. FIG. 26A is a western blot showing levels of cell cycle inhibitor proteins (p130, p27, p21, p16), retinoblastoma protein (Rb), and cyclin proteins (CCNE1, CCNE2, CCNA2) in small cell lung cancer (SCLC) model H69 cells administered vehicle (control) or increasing concentrations (30 nM, 100 nM, 300 nM, 10 mM) of Compound I. β-actin is included as an input-loading control. FIG. 26B is a western blot showing levels of cell cycle inhibitor proteins (p130, p27, p21, p16), cyclin-dependent kinase 2 (CDK2), and cyclin proteins (CCNE1, CCNE2, CCNA2) in small cell lung cancer (SCLC) model H526 cells administered vehicle (control) or increasing concentrations (30 nM, 100 nM, 300 nM, 10 mM) of Compound I. β-actin is included as an input-loading control. FIG. 29C is a western blot showing levels of cell cycle inhibitor proteins (p130, p27, p21, p16), cyclin-dependent kinase 2 (CDK2), and cyclin proteins (CCNE1, CCNE2, CCNA2) in small cell lung cancer (SCLC) model Hs68 cells administered vehicle (control) or increasing concentrations (30 nM, 100 nM, 300 nM, 10 mM) of Compound I. β-actin is included as an input-loading control.

FIG. 27A-D shows that Compound I activates caspase 3/7 in a temporal dependent manner in small cell lung cancer (SCLC) model H526 cells. Experimental methods for these experiments are found in Example 5. FIG. 27A is a grouped line graph illustrating Caspase 3/7 activation ratio to control in H526 cells on the y-axis and staurosporine (circle), Compound I (square), or PF-07104091 (triangle) molar (M) concentration at 24 hours on the x-axis. Listed below the graph are the caspase 3/7 activation IC₅₀ values for each compound administered. FIG. 27B is a grouped line graph illustrating Caspase 3/7 activation ratio to control in H526 cells on the y-axis and staurosporine (circle), Compound I (square), or PF-07104091 (triangle) molar (M) concentration at 48 hours on the x-axis. Listed below the graph are the caspase 3/7 activation IC₅₀ values for each compound administered. FIG. 27C is a grouped line graph illustrating Caspase 3/7 activation ratio to control in H526 cells on the y-axis and staurosporine (circle), Compound I (square), or PF-07104091 (triangle) molar (M) concentration at 72 hours on the x-axis. Listed below the graph are the caspase 3/7 activation IC₅₀ values for each compound administered. FIG. 27D is a table listing the caspase 3/7 activation IC₅₀ values in nanomolar (nM) units for each compound administered.

FIG. 28A-C shows H526 cell cycle changes and caspase 3/7 activation following Compound I and cisplatin co-administration. The symbol “>” indicates a sequential, staggered co-administration of the molecules indicated. The symbol “+” indicates two molecules were simultaneously administered. Experimental methods for these experiments are found in Example 5. FIG. 28A is a flow diagram showing the sequence of the experiment. FIG. 28B is a bar graph showing the percent of cells illustrated on the y-axis in different cell cycle phases, including DNA content <2N, G0/G1 phase, S phase, or G2/M phase, for different treatment conditions illustrated on the x-axis. FIG. 28C is a bar graph showing yH2AX ratio to control levels on the y-axis for different treatment conditions illustrated on the x-axis.

FIG. 29A-C shows that Compound I synergizes with doxorubicin to inhibit certain small cell lung cancer (SCLC) model cells. Experimental methods for these experiments are found in Example 5. FIG. 29A is a grouped line graph illustrating H526 cell number ratio to control on the y-axis and either Compound I (square), doxorubicin (triangle), or Compound I in combination with doxorubicin (diamond) molar (M) treatment concentration on the x-axis. FIG. 29B is a grouped line graph illustrating H69 cell number ratio to control on the y-axis and either Compound I (square), doxorubicin (triangle), or Compound I in combination with doxorubicin (diamond) molar (M) treatment concentration on the x-axis. FIG. 29C is a grouped line graph illustrating SHP77 cell number ratio to control on the y-axis and either Compound I (square), doxorubicin (triangle), or Compound I in combination with doxorubicin (diamond) molar (M) treatment concentration on the x-axis.

FIG. 30A-C shows that Compound I synergizes with camptothecin to inhibit small cell lung cancer (SCLC) model H526, H69, and SHP77 cells. Experimental methods for these experiments are found in Example 5. FIG. 30A is a grouped line graph illustrating H526 cell number ratio to control on the y-axis and either Compound I (square), camptothecin (triangle), or Compound I in combination with camptothecin (upside-down triangle) molar (M) treatment concentration on the x-axis. FIG. 30B is a grouped line graph illustrating H69 cell number ratio to control on the y-axis and either Compound I (square), camptothecin (triangle), or Compound I in combination with camptothecin (upside-down triangle) molar (M) treatment concentration on the x-axis. FIG. 30C is a grouped line graph illustrating SHP77 cell number ratio to control on the y-axis and either Compound I (square), camptothecin (triangle), or Compound I in combination with camptothecin (upside-down triangle) molar (M) treatment concentration on the x-axis.

FIG. 31A-G shows that Compound I has in vivo potency in a mouse hollow fiber assay comprising three tumor cell lines including OVCAR-3 (ovarian), MKN-1 (gastric), and HCC-1569 (breast). Mice were administered in different treatment groups: Group 1 (G1) administered vehicle twice per day at 10 ml/kg; Group 2 (G2) administered Compound I twice per day at 75 mg/kg, 10 ml/kg; Group 3 (G3) administered Compound I twice per day at 100 mg/kg, 10 ml/kg; Group 4 (G4) administered Compound I twice per day at 150 mg/kg, 10 ml/kg. Experimental methods for these experiments are found in Example 6. FIG. 31A is a grouped line plot showing mean animal weight in grams (g) on the y-axis over the course of a treatment schedule comprising the administration of different concentrations of Compound I. Female NMRI nude mice were implanted both subcutaneously and intraperitoneally with HCC-1569, MKN1, and OVCAR3 tumor cell-loaded Hollow Fibers on Day 0. Data are displayed as means+/−SEM. The number of animals alive on Day 0 (implantation) and on Day 16 (necropsy) in each group is shown in parentheses in the legend. FIG. 31B-C shows that Compound I induces changes in cancer cell viability in breast cancer tumor model HCC-1569 cell-implanted mice. HCC-1569 cell loaded Hollow Fibers were implanted both subcutaneously (left portion of graph) and intraperitoneally (right portion of graph) into female NMRI nude mice on day 0. Hollow Fibers were collected during necropsy on Day 16 and analyzed using a CellTiter Glo assay. FIG. 31B illustrates the photons/second mean values captured of CellTiter-Glo reagent from HCC-1569 Hollow Fibers on Day 16 on the y-axis and different treatment groups on the x-axis. FIG. 31C illustrates individual replicate photons/second values on the y-axis for different treatment groups on the x-axis. Error bars indicate interquartile range. FIG. 31D-E shows that Compound I induces changes in cancer cell viability in gastric cancer tumor model MKN-1 cell-implanted mice. MKN-1 cell loaded Hollow Fibers were implanted both subcutaneously (left portion of graph) and intraperitoneally (right portion of graph) into female NMRI nude mice on day 0. Hollow Fibers were collected during necropsy on Day 16 and analyzed using a CellTiter Glo assay. FIG. 31D illustrates the photons/second mean values captured of CellTiter-Glo reagent from MKN-1 Hollow Fibers on Day 16 on the y-axis and different treatment groups on the x-axis. FIG. 31E illustrates individual replicate photons/second values on the y-axis for different treatment groups on the x-axis. Error bars indicate interquartile range. FIG. 31F-G shows that Compound I induces changes in cancer cell viability in ovarian cancer tumor model OVCAR-3 cell-implanted mice. OVCAR-3 cell loaded Hollow Fibers were implanted both subcutaneously (left portion of graph) and intraperitoneally (right portion of graph) into female NMRI nude mice on day 0. Hollow Fibers were collected during necropsy on Day 16 and analyzed using a CellTiter Glo assay. FIG. 31F illustrates the photons/second mean values captured of CellTiter-Glo reagent from OVCAR-3 Hollow Fibers on Day 16 on the y-axis and different treatment groups on the x-axis. FIG. 31G illustrates individual replicate photons/second values on the y-axis for different treatment groups on the x-axis. Error bars indicate interquartile range.

FIG. 32 is a XRPD diffractogram of the crystalline Free Base Pattern 1. The diffractogram was obtained as described in Example 10 and 2-theta values with relative intensities are given in Table 14.

FIG. 33 is a XRPD diffractogram of the crystalline Free Base Pattern 2. The diffractogram was obtained as described in Example 10 and 2-theta values with relative intensities are given in Table 15.

FIG. 34 is a XRPD diffractogram of the crystalline Free Base Pattern 3. The diffractogram was obtained as described in Example 10 and 2-theta values with relative intensities are given in Table 16.

FIG. 35 is a XRPD diffractogram of the crystalline HCl Salt Pattern 1. The diffractogram was obtained as described in Example 10 and 2-theta values with relative intensities are given in Table 17.

FIG. 36 is a XRPD diffractogram of the crystalline HCl Salt Pattern 2. The diffractogram was obtained as described in Example 10 and 2-theta values with relative intensities are given in Table 18.

FIG. 37 is a XRPD diffractogram of the crystalline HCl Salt Pattern 3. The diffractogram was obtained as described in Example 10 and 2-theta values with relative intensities are given in Table 19.

FIG. 38 is a XRPD diffractogram of the crystalline Sulfuric Acid Salt Pattern 1. The diffractogram was obtained as described in Example 10 and 2-theta values with relative intensities are given in Table 20.

FIG. 39 is a XRPD diffractogram of the crystalline Sulfuric Acid Salt Pattern 1*. The diffractogram was obtained as described in Example 10 and 2-theta values with relative intensities are given in Table 21.

FIG. 40 is a XRPD diffractogram of the crystalline Sulfuric Acid Salt Pattern 2. The diffractogram was obtained as described in Example 10 and 2-theta values with relative intensities are given in Table 22.

FIG. 41 is a XRPD diffractogram of the crystalline Sulfuric Acid Salt Pattern 3. The diffractogram was obtained as described in Example 10 and 2-theta values with relative intensities are given in Table 23.

FIG. 42 is a XRPD diffractogram of the crystalline Sulfuric Acid Salt Pattern 4. The diffractogram was obtained as described in Example 10 and 2-theta values with relative intensities are given in Table 24.

FIG. 43 is a XRPD diffractogram of the crystalline Sulfuric Acid Salt Pattern 5. The diffractogram was obtained as described in Example 10 and 2-theta values with relative intensities are given in Table 25.

FIG. 44 is a XRPD diffractogram of the crystalline Sulfuric Acid Salt Pattern 6. The diffractogram was obtained as described in Example 10 and 2-theta values with relative intensities are given in Table 26.

FIG. 45 is a XRPD diffractogram of the crystalline Sulfuric Acid Salt Pattern 7. The diffractogram was obtained as described in Example 10 and 2-theta values with relative intensities are given in Table 27.

FIG. 46 is a XRPD diffractogram of the crystalline Methanesulfonic Acid Salt Pattern 1. The diffractogram was obtained as described in Example 10 and 2-theta values with relative intensities are given in Table 28.

FIG. 47 is a XRPD diffractogram of the crystalline Methanesulfonic Acid Salt Pattern 2. The diffractogram was obtained as described in Example 10 and 2-theta values with relative intensities are given in Table 29.

FIG. 48 is a XRPD diffractogram of the crystalline Methanesulfonic Acid Salt Pattern 3. The diffractogram was obtained as described in Example 10 and 2-theta values with relative intensities are given in Table 30.

FIG. 49 is a XRPD diffractogram of the crystalline Methanesulfonic Acid Salt Pattern 4. The diffractogram was obtained as described in Example 10 and 2-theta values with relative intensities are given in Table 31.

FIG. 50 is a XRPD diffractogram of the crystalline Methanesulfonic Acid Salt Pattern 5. The diffractogram was obtained as described in Example 10 and 2-theta values with relative intensities are given in Table 32.

FIG. 51 is a XRPD diffractogram of the crystalline Maleic Acid Salt Pattern 1. The diffractogram was obtained as described in Example 10 and 2-theta values with relative intensities are given in Table 33.

FIG. 52 is a XRPD diffractogram of the crystalline Maleic Acid Salt Pattern 1*. The diffractogram was obtained as described in Example 10 and 2-theta values with relative intensities are given in Table 34.

FIG. 53 is a XRPD diffractogram of the crystalline Maleic Acid Salt Pattern 2. The diffractogram was obtained as described in Example 10 and 2-theta values with relative intensities are given in Table 35.

FIG. 54 is a XRPD diffractogram of the crystalline Maleic Acid Salt Pattern 3. The diffractogram was obtained as described in Example 10 and 2-theta values with relative intensities are given in Table 36.

FIG. 55 is a XRPD diffractogram of the crystalline Maleic Acid Salt Pattern 3*. The diffractogram was obtained as described in Example 10 and 2-theta values with relative intensities are given in Table 37.

FIG. 56 is a XRPD diffractogram of the crystalline Maleic Acid Salt Pattern 4. The diffractogram was obtained as described in Example 10 and 2-theta values with relative intensities are given in Table 38.

FIG. 57 is a XRPD diffractogram of the crystalline Maleic Acid Salt Pattern 5. The diffractogram was obtained as described in Example 10 and 2-theta values with relative intensities are given in Table 39.

FIG. 58 is a XRPD diffractogram of the crystalline Phosphoric Acid Salt Pattern 1. The diffractogram was obtained as described in Example 10 and 2-theta values with relative intensities are given in Table 40.

FIG. 59 is a XRPD diffractogram of the crystalline Phosphoric Acid Salt Pattern 2. The diffractogram was obtained as described in Example 10 and 2-theta values with relative intensities are given in Table 41.

FIG. 60 is a XRPD diffractogram of the crystalline Phosphoric Acid Salt Pattern 3. The diffractogram was obtained as described in Example 10 and 2-theta values with relative intensities are given in Table 42.

FIG. 61 is a XRPD diffractogram of the crystalline Phosphoric Acid Salt Pattern 4. The diffractogram was obtained as described in Example 10 and 2-theta values with relative intensities are given in Table 43.

FIG. 62 is a XRPD diffractogram of the crystalline Phosphoric Acid Salt Pattern 5. The diffractogram was obtained as described in Example 10 and 2-theta values with relative intensities are given in Table 44.

FIG. 63 is a XRPD diffractogram of the crystalline Phosphoric Acid Salt Pattern 6. The diffractogram was obtained as described in Example 10 and 2-theta values with relative intensities are given in Table 45.

FIG. 64 is a XRPD diffractogram of the crystalline Phosphoric Acid Salt Pattern 6*. The diffractogram was obtained as described in Example 10 and 2-theta values with relative intensities are given in Table 46.

FIG. 65 is a XRPD diffractogram of the crystalline Phosphoric Acid Salt Pattern 7. The diffractogram was obtained as described in Example 10 and 2-theta values with relative intensities are given in Table 47.

FIG. 66 is a XRPD diffractogram of the crystalline Phosphoric Acid Salt Pattern 7*. The diffractogram was obtained as described in Example 10 and 2-theta values with relative intensities are given in Table 48.

FIG. 67 is a XRPD diffractogram of the crystalline Phosphoric Acid Salt Pattern 8. The diffractogram was obtained as described in Example 10 and 2-theta values with relative intensities are given in Table 49.

FIG. 68 is a XRPD diffractogram of the crystalline Phosphoric Acid Salt Pattern 8*. The diffractogram was obtained as described in Example 10 and 2-theta values with relative intensities are given in Table 50.

FIG. 69 is a XRPD diffractogram of the crystalline (+)-L-Tartaric Acid Salt Pattern 1. The diffractogram was obtained as described in Example 10 and 2-theta values with relative intensities are given in Table 51.

FIG. 70 is a XRPD diffractogram of the crystalline (+)-L-Tartaric Acid Salt Pattern 1*. The diffractogram was obtained as described in Example 10 and 2-theta values with relative intensities are given in Table 52.

FIG. 71 is a XRPD diffractogram of the crystalline (+)-L-Tartaric Acid Salt Pattern 2. The diffractogram was obtained as described in Example 10 and 2-theta values with relative intensities are given in Table 53.

FIG. 72 is a XRPD diffractogram of the crystalline Citric Acid Salt Pattern 1. The diffractogram was obtained as described in Example 10 and 2-theta values with relative intensities are given in Table 54.

FIG. 73 is a XRPD diffractogram of the crystalline Citric Acid Salt Pattern 2. The diffractogram was obtained as described in Example 10 and 2-theta values with relative intensities are given in Table 55.

FIG. 74 is a XRPD diffractogram of the crystalline Citric Acid Salt Pattern 4. The diffractogram was obtained as described in Example 10 and 2-theta values with relative intensities are given in Table 56.

FIG. 75 is a XRPD diffractogram of the crystalline Hydrobromic Acid Salt Pattern 1. The diffractogram was obtained as described in Example 10 and 2-theta values with relative intensities are given in Table 57.

FIG. 76 is a XRPD diffractogram of the crystalline Hydrobromic Acid Salt Pattern 2. The diffractogram was obtained as described in Example 10 and 2-theta values with relative intensities are given in Table 58.

FIG. 77 is a XRPD diffractogram of the crystalline Benzenesulfonic Acid Salt Pattern 1. The diffractogram was obtained as described in Example 10 and 2-theta values with relative intensities are given in Table 59.

FIG. 78 is a XRPD diffractogram of the crystalline Benzenesulfonic Acid Salt Pattern 2. The diffractogram was obtained as described in Example 10 and 2-theta values with relative intensities are given in Table 60.

FIG. 79 is a XRPD diffractogram of the crystalline Benzenesulfonic Acid Salt Pattern 3. The diffractogram was obtained as described in Example 10 and 2-theta values with relative intensities are given in Table 61.

FIG. 80 is a XRPD diffractogram of the crystalline Benzenesulfonic Acid Salt Pattern 3*. The diffractogram was obtained as described in Example 10 and 2-theta values with relative intensities are given in Table 62.

FIG. 81 is a thermogravimetric/differential scanning calorimetry (TG/DSC) thermogram of Sulfuric Acid Pattern 1. The TG/DSC thermogram was obtained as described in Example 13 with results presented in Table 63.

FIG. 82 is a thermogravimetric/differential scanning calorimetry (TG/DSC) thermogram of Sulfuric Acid Pattern 2. The TG/DSC thermogram was obtained as described in Example 13 with results presented in Table 63.

FIG. 83 is a thermogravimetric/differential scanning calorimetry (TG/DSC) thermogram of Sulfuric Acid Pattern 3. The TG/DSC thermogram was obtained as described in Example 13 with results presented in Table 63.

FIG. 84 is a thermogravimetric/differential scanning calorimetry (TG/DSC) thermogram of Sulfuric Acid Pattern 4. The TG/DSC thermogram was obtained as described in Example 13 with results presented in Table 63.

FIG. 85 is a thermogravimetric/differential scanning calorimetry (TG/DSC) thermogram of Sulfuric Acid Pattern 5. The TG/DSC thermogram was obtained as described in Example 13 with results presented in Table 63.

FIG. 86 is a thermogravimetric/differential scanning calorimetry (TG/DSC) thermogram of Sulfuric Acid Pattern 6. The TG/DSC thermogram was obtained as described in Example 13 with results presented in Table 63.

FIG. 87 is a thermogravimetric/differential scanning calorimetry (TG/DSC) thermogram of Methanesulfonic Acid Pattern 1. The TG/DSC thermogram was obtained as described in Example 13 with results presented in Example 23 and Table 64.

FIG. 88 is a thermogravimetric/differential scanning calorimetry (TG/DSC) thermogram of Methanesulfonic Acid Pattern 2. The TG/DSC thermogram was obtained as described in Example 13 with results presented in Example 23 and Table 64.

FIG. 89 is a thermogravimetric/differential scanning calorimetry (TG/DSC) thermogram of Methanesulfonic Acid Pattern 3. The TG/DSC thermogram was obtained as described in Example 13 with results presented in Example 23 and Table 64.

FIG. 90 is a thermogravimetric/differential scanning calorimetry (TG/DSC) thermogram of Methanesulfonic Acid Pattern 4. The TG/DSC thermogram was obtained as described in Example 13 with results presented in Example 23 and Table 64.

FIG. 91 is a thermogravimetric/differential scanning calorimetry (TG/DSC) thermogram of Maleic Acid Pattern 1. The TG/DSC thermogram was obtained as described in Example 13 with results presented in Example 24 and Table 65.

FIG. 92 is a thermogravimetric/differential scanning calorimetry (TG/DSC) thermogram of Maleic Acid Pattern 2. The TG/DSC thermogram was obtained as described in Example 13 with results presented in Example 24 and Table 65.

FIG. 93 is a thermogravimetric/differential scanning calorimetry (TG/DSC) thermogram of Maleic Acid Pattern 3. The TG/DSC thermogram was obtained as described in Example 13 with results presented in Example 24 and Table 65.

FIG. 94 is a thermogravimetric/differential scanning calorimetry (TG/DSC) thermogram of Maleic Acid Pattern 4. The TG/DSC thermogram was obtained as described in Example 13 with results presented in Example 24 and Table 65.

FIG. 95 is a thermogravimetric/differential scanning calorimetry (TG/DSC) thermogram of Maleic Acid Pattern 5. The TG/DSC thermogram was obtained as described in Example 13 with results presented in Example 24 and Table 65.

FIG. 96 is a thermogravimetric/differential scanning calorimetry (TG/DSC) thermogram of Phosphoric Acid Pattern 1. The TG/DSC thermogram was obtained as described in Example 13 with results presented in Example 25 and Table 66.

FIG. 97 is a thermogravimetric/differential scanning calorimetry (TG/DSC) thermogram of Phosphoric Acid Pattern 2. The TG/DSC thermogram was obtained as described in Example 13 with results presented in Example 25 and Table 66.

FIG. 98 is a thermogravimetric/differential scanning calorimetry (TG/DSC) thermogram of Phosphoric Acid Pattern 3. The TG/DSC thermogram was obtained as described in Example 13 with results presented in Example 25 and Table 66.

FIG. 99 is a thermogravimetric/differential scanning calorimetry (TG/DSC) thermogram of Phosphoric Acid Pattern 4. The TG/DSC thermogram was obtained as described in Example 13 with results presented in Example 25 and Table 66.

FIG. 100 is a thermogravimetric/differential scanning calorimetry (TG/DSC) thermogram of Phosphoric Acid Pattern 7*. The TG/DSC thermogram was obtained as described in Example 13 with results presented in Example 25 and Table 66.

FIG. 101 is a thermogravimetric/differential scanning calorimetry (TG/DSC) thermogram of Phosphoric Acid Pattern 8*. The TG/DSC thermogram was obtained as described in Example 13 with results presented in Example 25 and Table 66.

FIG. 102 is a thermogravimetric/differential scanning calorimetry (TG/DSC) thermogram of (+)-L-Tartaric Acid Pattern 1. The TG/DSC thermogram was obtained as described in Example 13 with results presented in Example 26 and Table 67.

FIG. 103 is a thermogravimetric/differential scanning calorimetry (TG/DSC) thermogram of (+)-L-Tartaric Acid Pattern 2. The TG/DSC thermogram was obtained as described in Example 13 with results presented in Example 26 and Table 67.

FIG. 104 is a thermogravimetric/differential scanning calorimetry (TG/DSC) thermogram of Citric Acid Pattern 1. The TG/DSC thermogram was obtained as described in Example 13 with results presented in Example 27 and Table 68.

FIG. 105 is a thermogravimetric/differential scanning calorimetry (TG/DSC) thermogram of Citric Acid Pattern 1. The TG/DSC thermogram was obtained as described in Example 13 with results presented in Example 27 and Table 68.

FIG. 106 is a thermogravimetric/differential scanning calorimetry (TG/DSC) thermogram of Citric Acid Pattern 1. The TG/DSC thermogram was obtained as described in Example 13 with results presented in Example 27 and Table 68.

FIG. 107 is a thermogravimetric/differential scanning calorimetry (TG/DSC) thermogram of Hydrobromic Acid Pattern 1. The TG/DSC thermogram was obtained as described in Example 13 with results presented in Example 28 and Table 69.

FIG. 108 is a thermogravimetric/differential scanning calorimetry (TG/DSC) thermogram of Hydrobromic Acid Pattern 1. The TG/DSC thermogram was obtained as described in Example 13 with results presented in Example 28 and Table 69.

FIG. 109 is a thermogravimetric/differential scanning calorimetry (TG/DSC) thermogram of Benzenesulfonic Acid Pattern 1. The TG/DSC thermogram was obtained as described in Example 13 with results presented in Example 29 and Table 70.

FIG. 110 is a thermogravimetric/differential scanning calorimetry (TG/DSC) thermogram of Benzenesulfonic Acid Pattern 2. The TG/DSC thermogram was obtained as described in Example 13 with results presented in Example 29 and Table 70.

FIG. 111 is a thermogravimetric/differential scanning calorimetry (TG/DSC) thermogram of Benzenesulfonic Acid Pattern 3. The TG/DSC thermogram was obtained as described in Example 13 with results presented in Example 29 and Table 70.

FIG. 112 is a thermogravimetric/differential scanning calorimetry (TG/DSC) thermogram of HCl Salt Pattern 1. The TG/DSC thermogram was obtained as described in Example 13 with results presented in Example 30.

FIG. 113 is a thermogravimetric/differential scanning calorimetry (TG/DSC) thermogram of HCl Salt Pattern 2. The TG/DSC thermogram was obtained as described in Example 13 with results presented in Example 30 and Table 72.

FIG. 114 is a differential scanning calorimetry (DSC) thermogram of HCl Salt Pattern 1. The DSC thermogram was obtained as described in Example 14 with results presented in Example 30.

FIG. 115 is a Dynamic Vapor Sorption (DVS) analysis showing the results from a moisture sorption experiment of HCl Pattern 1. DVS analysis of HCl Pattern 1 was performed as described in Example 15. DVS analysis revealed the material to be moderately hygroscopic with an uptake of 5.15% and 5.10% in the first and second sorption cycles respectively. The results of the study are presented in Example 30.

FIG. 116 is a Dynamic Vapor Sorption (DVS) Kinetic Plot of HCl Pattern 1. DVS of HCl Pattern 1 was obtained as described in Example 15. DVS analysis revealed the material to be moderately hygroscopic with an uptake of 5.15% and 5.10% in the first and second sorption cycles respectively. The results of the study are presented in Example 30.

FIG. 117 is a thermogravimetric/differential scanning calorimetry (TG/DSC) thermogram of Sulfuric Acid Pattern 7. The TG/DSC thermogram was obtained as described in Example 13 with results presented in Example 34.

FIG. 118 is a thermogravimetric/differential scanning calorimetry (TG/DSC) thermogram of Phosphate Pattern 7*. The TG/DSC thermogram was obtained as described in Example 13 with results presented in Table 79.

FIG. 119 is a differential scanning calorimetry (DSC) thermogram of Phosphate Pattern 7*. The DSC thermogram was obtained as described in Example 14 with results presented in Table 79.

FIG. 120 is a Dynamic Vapor Sorption (DVS) analysis showing the results from a moisture sorption experiment of Phosphate Pattern 7*. DVS analysis of Phosphate Pattern 7* was performed as described in Example 15. DVS analysis revealed the material to be moderately hygroscopic with an uptake of 4.76% and 5.18% in the first and second sorption cycles respectively. The results of the study are presented in Table 79.

FIG. 121 is a Dynamic Vapor Sorption (DVS) Kinetic Plot of Phosphate Pattern 7* DVS of Phosphate Pattern 7* was obtained as described in Example 15. The results of the study are presented in Table 79.

FIG. 122 is PLM image of HCl Pattern 1.

FIG. 123 is PLM image of Phosphate Pattern 7*.

FIG. 124 a plot showing the effect of Compound I on hERG current. Experimental conditions for this experiment are found in Example 40. Data points (triangles) show percent hERG block values represented on the y-axis at certain concentrations of Compound I represented on the x-axis.

FIG. 125 is a plot showing Compound I inhibition of COX1. Experimental conditions for this experiment are found in Example 40. Data points (triangles) show percent of control enzyme inhibition of the binding of diclofenac ligand for COX1 represented on the y-axis at certain concentrations of Compound I represented on the x-axis.

FIG. 126 is a plot showing Compound I inhibition of COX2. Experimental conditions for this experiment are found in Example 40. Data points (triangles) show percent of control enzyme inhibition of the binding of NS398 ligand for COX2 represented on the y-axis at certain concentrations of Compound I represented on the x-axis.

FIG. 127 is a plot showing Compound I inhibition of CYP1A2. Experimental conditions for this experiment are found in Example 41. Data points (triangles) show percent of control enzyme inhibition of the formation of hydroxytacrine for the CYP1A2-catalyzed metabolism of tacrine represented on the y-axis at certain concentrations of Compound I represented on the x-axis.

FIG. 128 is a plot showing Compound I inhibition of CYP2B6. Experimental conditions for this experiment are found in Example 41. Data points (triangles) show percent of control enzyme inhibition of the formation of hydroxybuproprion for the CYP2B6-catalyzed metabolism of buproprion represented on the y-axis at certain concentrations of Compound I represented on the x-axis.

FIG. 129 is a plot showing Compound I inhibition of CYP2C8. Experimental conditions for this experiment are found in Example 41. Data points (triangles) show percent of control enzyme inhibition of the formation of desethylamodiquine for the CYP2C8-catalyzed metabolism of amodiaquine represented on the y-axis at certain concentrations of Compound I represented on the x-axis.

FIG. 130 is a plot showing Compound I inhibition of CYP2C9. Experimental conditions for this experiment are found in Example 41. Data points (triangles) show percent of control enzyme inhibition of the formation of 4-hydroxytolbutamide for the CYP2C9-catalyzed metabolism of tolbutamide represented on the y-axis at certain concentrations of Compound I represented on the x-axis.

FIG. 131 is a plot showing Compound I inhibition of CYP2C19. Experimental conditions for this experiment are found in Example 41. Data points (triangles) show percent of control enzyme inhibition of the formation of 4-hydroxymephenytoin for the CYP2C19-catalyzed metabolism of mephenytoin represented on the y-axis at certain concentrations of Compound I represented on the x-axis.

FIG. 132 is a plot showing Compound I inhibition of CYP2D6. Experimental conditions for this experiment are found in Example 41. Data points (triangles) show percent of control enzyme inhibition of the formation of dextrorphan for the CYP2D6-catalyzed metabolism of dextromethorphan represented on the y-axis at certain concentrations of Compound I represented on the x-axis.

FIG. 133 is a plot showing Compound I inhibition of CYP3A4. Experimental conditions for this experiment are found in Example 41. Data points (triangles) show percent of control enzyme inhibition of the formation of 1-hydroxymidazolam for the CYP3A4-catalyzed metabolism of midazolam represented on the y-axis at certain concentrations of Compound I represented on the x-axis.

FIG. 134 is a plot showing Compound I inhibition of CYP3A4. Experimental conditions for this experiment are found in Example 41. Data points (triangles) show percent of control enzyme inhibition of the formation of 6β-hydroxy-testosterone for the CYP3A4-catalyzed metabolism of testosterone represented on the y-axis at certain concentrations of Compound I represented on the x-axis.

FIG. 135 is a plot showing Compound I inhibition of BCRP. Experimental conditions for this experiment are found in Example 41. Data points (triangles) show percent of control enzyme inhibition of estrone-3-sulfate transport mediated by BCRP represented on the y-axis at certain concentrations of Compound I represented on the x-axis.

FIG. 136 is a plot showing Compound I inhibition of MATE1. Experimental conditions for this experiment are found in Example 41. Data points (triangles) show percent of control enzyme inhibition of metformin transport mediated by MATE1 represented on the y-axis at certain concentrations of Compound I represented on the x-axis.

FIG. 137 is a plot showing Compound I inhibition of MATE2-K. Experimental conditions for this experiment are found in Example 41. Data points (triangles) show percent of control enzyme inhibition of metformin transport mediated by MATE2-K represented on the y-axis at certain concentrations of Compound I represented on the x-axis.

FIG. 138 is a plot showing Compound I inhibition of OAT1. Experimental conditions for this experiment are found in Example 41. Data points (triangles) show percent of control enzyme inhibition of tenofovir transport mediated by OAT1 represented on the y-axis at certain concentrations of Compound I represented on the x-axis.

FIG. 139 is a plot showing Compound I inhibition of OAT3. Experimental conditions for this experiment are found in Example 41. Data points (triangles) show percent of control enzyme inhibition of estrone-3-sulfate transport mediated by OAT3 represented on the y-axis at certain concentrations of Compound I represented on the x-axis.

FIG. 140 is a plot showing Compound I inhibition of OAT1PIB1. Experimental conditions for this experiment are found in Example 41. Data points (triangles) show percent of control enzyme inhibition of estradiol-17-β-glucuronide transport mediated by OATIP1B1 represented on the y-axis at certain concentrations of Compound I represented on the x-axis.

FIG. 141 is a plot showing Compound I inhibition of OAT1P1B3. Experimental conditions for this experiment are found in Example 41. Data points (triangles) show percent of control enzyme inhibition of cholecystokinin octapeptide transport mediated by OAT1P1B3 represented on the y-axis at certain concentrations of Compound I represented on the x-axis.

FIG. 142 is a plot showing Compound I inhibition of OCT1. Experimental conditions for this experiment are found in Example 41. Data points (triangles) show percent of control enzyme inhibition of sumatriptan transport mediated by OCT1 represented on the y-axis at certain concentrations of Compound I represented on the x-axis.

FIG. 143 is a plot showing Compound I inhibition of OCT2. Experimental conditions for this experiment are found in Example 41. Data points (triangles) show percent of control enzyme inhibition of metformin transport mediated by OCT2 represented on the y-axis at certain concentrations of Compound I represented on the x-axis.

FIG. 144 is a plot showing Compound I phototoxicity in Balb-c3T3 mice. Experimental conditions for this experiment are found in Example 42. Cell viability was measured as percent of live cells on the y-axis over different concentrations of Compound I (μg/mL) represented on the x-axis in the dark (closed circle) and light (open square).

FIG. 145 show culture plate images taken during a crystal violet colony assay showing Compound I prolongs the time to CDK4/6 inhibitor resistance in ER+ breast cancer. Experimental conditions for this experiment are found in Example 43. T47D human ER+ breast carcinoma cell colonies and BT474 human ER+ breast carcinoma cell colonies were treated with Compound I alone or in combination with CDK4/6 inhibitor (CDK4/6i) palbociclib or abemaciclib from Weeks 3-8.

FIG. 146A-146H shows Compound I reduces Rb phosphorylation and cyclin A2 levels in tumors and induces tumor regression and stasis in ovarian and gastric CCNE1-amplified animal preclinical cancer models. Experimental conditions for these experiments are found in Example 44. FIG. 146A is a plot showing cell line-derived tumor xenograft (CDX) OVCAR3 ovarian mouse preclinical model administered vehicle (circle) or Compound I at 200 mg/kg once per day (QD) (square) or 100 mg/kg twice per day (BID) (triangle). Tumor volume (mm³) is represented on the y-axis and days on study is represented on the x-axis. Mice were treated for 42 days and showed tumor stasis with 100 BID treatment or 89% TGI with 200 QD treatment. n=10, no body weights loss was >5% at any point. FIG. 146B is a plot showing patient-derived tumor xenograft (PDX) OV5398 ovarian mouse preclinical model administered vehicle (circle) or Compound I at 200 mg/kg once per day (QD) (triangle) or 100 mg/kg twice per day (BID) (diamond). Tumor volume (mm³) is represented on the y-axis and days on study is represented on the x-axis. Mice were treated for 56 days and showed tumor regression in both treatment groups. n=10, no body weights loss was >5% at any point. FIG. 146C is a plot showing patient-derived tumor xenograft (PDX) GA0103 gastric mouse preclinical model administered vehicle (circle) or Compound I at 100 mg/kg twice per day (BID) (triangle). Tumor volume (mm³) is represented on the y-axis and days on study is represented on the x-axis. Mice were treated for 56 days and showed tumor stasis with 100 BID treatment. n=8, no body weights loss was >5% at any point. FIG. 146D is a plot showing patient-derived tumor xenograft (PDX) GA0114 gastric mouse preclinical model administered vehicle (circle) or Compound I at 100 mg/kg twice per day (BID) (triangle). Tumor volume (mm³) is represented on the y-axis and days on study is represented on the x-axis. Mice were treated for 35 days and showed 95% TGI with 100 BID treatment. N=8, no body weights loss was >5% at any point. FIG. 146E is a western blot showing levels retinoblastoma protein (Rb), markers of phosphorylated Rb protein (pRb 807/811, pRb T821), and cyclin A2 (CCNA2) in OVCAR3 tumors extracted from the cell line-derived tumor xenograft (CDX) OVCAR3 ovarian mouse model either 2 hours or 24 hours post-final dose of control or Compound I. FIG. 146F is a western blot showing levels retinoblastoma protein (Rb), markers of phosphorylated Rb protein (pRb 807/811, pRb T821), and cyclin A2 (CCNA2) in OV5398 tumors extracted from the patient-derived tumor xenograft (PDX) OV5398 ovarian mouse model either 2 hours or 24 hours post-final dose of control or Compound I. FIG. 146G is a plot showing relative thymidine kinase (TK) activity levels as calculated by ELISA. TK activity is represented on the y-axis versus GA0103 PDX model mice receiving vehicle control or Compound I at 100 mpk BID. FIG. 146H is a plot showing relative TK activity levels as calculated by ELISA. TK activity levels as represented on the y-axis versus GA0114 PDX model mice receiving vehicle control or Compound I at 100 mpk BID.

FIG. 147A-147L shows Compound I in combination with a CDK4/6 inhibitor (CDK4/6i) restores sensitivity in cell lines resistant to CDK4/6i and/or anti-estrogen therapy. Experimental conditions for these experiments are found in Example 45. FIG. 147A is a bar graph showing multiple cell lines with resistance to CDK4/6 inhibitors and/or estrogen therapy show sensitivity to Compound I, CDK4/6 inhibitor combination. The bar graph illustrates cellular proliferation IC₅₀ values in nanomolar (nM) on the y-axis and different therapy-resistant daughter MCF7 or T47D luminal breast cancer cells on the x-axis. PAR: Non-4/6i resistant parental line; LYR: cell line resistant to abemaciclib; FR: cell line resistant to fulvestrant; LYFR: cell line resistant to abemaciclib and fulvestrant; PAR+LY: Parental line with 500 nM abemaciclib; PDR: cell line resistant to palbociclib. FIG. 147B is a grouped line graph illustrating breast cancer model T47D 2×DT cell number fold-change on the y-axis and Compound I nanomolar (nM) treatment concentration on the x-axis. Non-resistant parental (PAR) cell lines were treated with Compound I (circle). Parental cell lines previously administered 500 nM abemaciclib (PAR+LY) were administered abemaciclib in combination with Compound I (diamond). Abemaciclib-resistant (LYR) cells were administered abemaciclib in combination with Compound I (square). Fulvestrant-resistant (FR) cells were administered fulvestrant in combination with Compound I (right side up triangle). Abemaciclib- and fulvestrant-resistant (LYFR) cells were administered abemaciclib in combination with fulvestrant and Compound I (upside down triangle). FIG. 147C is a grouped line graph illustrating breast cancer model MCF7-M 2×DT cell number fold-change on the y-axis and Compound I nanomolar (nM) treatment concentration on the x-axis. Non-resistant parental (PAR) cell lines were treated with Compound I (circle). Parental cell lines previously administered 500 nM abemaciclib (PAR+LY) were administered abemaciclib in combination with Compound I (diamond). Abemaciclib-resistant (LYR) cells were administered abemaciclib in combination with Compound I (square). Fulvestrant-resistant (FR) cells were administered fulvestrant in combination with Compound I (right side up triangle). Abemaciclib- and fulvestrant-resistant (LYFR) cells were administered abemaciclib in combination with fulvestrant and Compound I (upside down triangle). FIG. 147D is a bar graph showing T47D cells that are resistant to abemaciclib are arrested in G1 when combined with Compound I. The bar graph illustrates the percentage of cells in different cell phases on the y-axis for different therapy-resistant daughter T47D cells illustrated on the x-axis. PAR: Non-4/6i resistant parental line; LYR: cell line resistant to abemaciclib; FR: cell line resistant to fulvestrant; LYFR: cell line resistant to abemaciclib and fulvestrant; PAR+LY: Parental line with 500 nM abemaciclib; PDR: cell line resistant to palbociclib. FIG. 147E is a flow cytometry plot quantifying the percent of non-resistant parental (PAR) cells in the S phase, G1 phase, or G2M phase following treatment with DMSO control. Quantification of FITC: BrdU stain intensity is represented on the y-axis and PI-A stain intensity is represented on the x-axis. FIG. 147F is a flow cytometry plot quantifying the percent of non-resistant parental (PAR) cells in the S phase, G1 phase, or G2M phase following treatment with abemaciclib. Quantification of FITC: BrdU stain intensity is represented on the y-axis and PI-A stain intensity is represented on the x-axis. FIG. 147G is a flow cytometry plot quantifying the percent of abemaciclib-resistant (LYR) cells in the S phase, G1 phase, or G2M phase following treatment with abemaciclib. Quantification of FITC: BrdU stain intensity is represented on the y-axis and PI-A stain intensity is represented on the x-axis. FIG. 147H is a flow cytometry plot quantifying the percent of abemaciclib- and fulvestrant-resistant (LYFR) cells in the S phase, G1 phase, or G2M phase following treatment with abemaciclib and fulvestrant. Quantification of FITC: BrdU stain intensity is represented on the y-axis and PI-A stain intensity is represented on the x-axis. FIG. 147I is a flow cytometry plot quantifying the percent of abemaciclib-resistant (LYR) cells in the S phase, G1 phase, or G2M phase following treatment with abemaciclib in combination with Compound I. Quantification of FITC: BrdU stain intensity is represented on the y-axis and PI-A stain intensity is represented on the x-axis. FIG. 147J is a flow cytometry plot quantifying the percent of abemaciclib- and fulvestrant-resistant (LYFR) cells in the S phase, G1 phase, or G2M phase following treatment with abemaciclib in combination with fulvestrant and Compound I. Quantification of FITC: BrdU stain intensity is represented on the y-axis and PI-A stain intensity is represented on the x-axis. FIG. 147K is a western blot showing Compound I in combination with a CDK4/6i prevents phosphorylation of Rb in therapy-resistant cells. The western blot showing levels retinoblastoma protein (Rb), markers of phosphorylated Rb protein (pRb S780), and Histone H3 loading control in response to DMSO or either abemaciclib and Compound I alone or in combination. Parent: Non-4/6i resistant parental line; LYR: cell line resistant to abemaciclib; FR: cell line resistant to fulvestrant; LYFR: cell line resistant to abemaciclib and fulvestrant. FIG. 147L is a western blot showing levels of phosphorylated retinoblastoma protein (pRb), and vinculin loading control in therapy-resistant cells. Therapy-resistant cells were administered DMSO, abemaciclib, fulvestrant, and/or Compound I. PAR: Non-4/6i resistant parental line; LYR: cell line resistant to abemaciclib; FR: cell line resistant to fulvestrant; LYFR: cell line resistant to abemaciclib and fulvestrant; PAR+LY: Parental line with 500 nM abemaciclib; +: drug administered; −: drug not administered.

FIG. 148A-148F shows beta-galactosidase staining of therapy-resistant cells treated with abemaciclib, fulvestrant, and/or Compound I. Experimental conditions for these experiments are found in Example 45. FIG. 148A is a micrograph showing beta-galactosidase staining of abemaciclib-resistant (LYR) cells treated with abemaciclib. FIG. 148B is a micrograph showing beta-galactosidase staining of abemaciclib-resistant (LYR) cells treated with abemaciclib in combination with Compound I. FIG. 148C is a micrograph showing beta-galactosidase staining of abemaciclib- and fulvestrant-resistant (LYFR) cells treated with abemaciclib in combination with fulvestrant. FIG. 148D is a micrograph showing beta-galactosidase staining of abemaciclib- and fulvestrant-resistant (LYFR) cells treated with abemaciclib in combination with fulvestrant and Compound I. FIG. 148E is a plot showing beta galactosidase activity in abemaciclib-resistant (LYR) cells. The quantification of the ratio of beta galactosidase staining area to cell area is represented on the y-axis in different treatment groups including abemaciclib+DMSO or abemaciclib+Compound I represented on the x-axis. FIG. 148F is a plot showing beta galactosidase activity in abemaciclib- and fulvestrant-resistant (LYFR) cells. The quantification of the ratio of beta galactosidase staining area to cell area is represented on the y-axis in different treatment groups including abemaciclib+fulvestrant+DMSO or abemaciclib+fulvestrant+Compound I represented on the x-axis.

FIG. 149 is a plot showing tumor volume fold changes in mice overexpressing HER2 (MMTV-rtTA/tetO-HER2) pre-treated with CDK4/6 inhibitor abemaciclib until resistance to treatment was observed. Experimental conditions for this experiment are found in Example 45. Mice were randomized then administered vehicle control (square), Compound I at 50* mg/kg twice per day (BID) (upside-down triangle), abemaciclib at 50 mg/kg twice per day (BID) (right side up triangle), or a combination of abemaciclib and Compound I at the doses and frequencies described above (diamond). Tumor volume fold change is represented on the y-axis and days on treatment is represented on the x-axis. * Compound I was originally dosed at 75 mg/kg BID but was reduced at day 9.

FIG. 150A-150C shows that estrogen receptor-positive (ER+) breast cancer model T47D cells are sensitive to a combination treatment comprising the administration of both Compound I and fulvestrant. Experimental conditions for these experiments are found in Example 46. FIG. 150A are images of culture plates containing T47D human breast carcinoma cell colonies under Compound I and/or fulvestrant treatment conditions versus vehicle control at Week 2 and 3. FIG. 150B is a bar graph illustrating average colony size for different treatment conditions on the y-axis compared to control at Week 2 and 3 indicated on the x-axis. FIG. 150C is a bar graph illustrating percent colony area for different treatment conditions on the y-axis compared to control at Week 2 and 3 indicated on the x-axis.

FIG. 151 illustrates the potent and selective CDK2 inhibitor Compound I which demonstrates robust anticancer activity in CCNE-amplified and CDK4/6 inhibitor-resistant cancers. Cyclin E1 (CCNE1) and E2 (CCNE2) are critical cell cycle regulators, that bind to CDK2 which phosphorylate retinoblastoma (Rb) to drive cancer cell proliferation via the transition from G1 to S phase of the cell cycle. Dysregulated CDK2 activity frequently occurs across a variety of human cancers with amplification/overexpression of CCNE1 and CCNE2 being the most frequent mechanism of dysregulated CDK2 activity and CDK4/6 inhibitor resistance. Compound I is a selective and potent CDK2 inhibitor that may provide clinical benefit and delay time to resistance to conventional therapies in patients with CDK2/cyclin E driven cancers. In CCNE1-amplified human ovarian and gastric cancer cell lines, Compound I potently inhibits retinoblastoma (Rb) phosphorylation, induces a G1 cell cycle arrest, and inhibits cellular proliferation. Compound I also provides potent anti-proliferative activity in luminal breast cancer cell lines that had been cultured in the presence of a CDK4/6 inhibitor for prolonged periods enabling drug resistance. In CCNE1-amplified models of breast, ovarian, and gastric carcinomas, Compound I inhibits Rb-phosphorylation and induces tumor regression and extended periods of tumor stasis.

### DETAILED DESCRIPTION

It has been surprisingly and unexpectedly discovered that Compound I or a pharmaceutically acceptable salt thereof or morphic form described herein, for example Pattern 1, is particularly effective at treating cancers which have amplified or overexpressed cyclin E, are resistant to CDK4/6 inhibitors either through acquired resistance or intrinsic resistance (e.g., SCLC), and/or are resistant to endocrine therapies, for example estrogen receptor degraders. Compound I can be used to treat difficult to treat cyclin E amplified cancers including CCNE1 amplified unresectable solid tumors and CCNE1 amplified platinum-resistant or platinum-refractory cancers. Non-limiting examples of favorable properties exhibited by Compound I include selective inhibition of CDK2, inhibition of CDK2 across several cyclin complex partners, substantially long residence time when complexed with CDK2, a robust safety profile when administered to healthy, non-cancerous cells, synergy with chemotherapeutic agents for greater anti-tumor potency, anti-tumor potency in CDK4/6 inhibitor-resistant cells, anti-tumor potency in endocrine inhibitor-resistant cells, anti-tumor potency in cells with amplified cyclin E activation and/or expression, a change in expression of cell cycle-related proteins, induction of a targeted DNA-damage response in neoplastic cells, durable tumor inhibition, and/or improved overall survival. The altered steady-state levels of proteins involved in the cell cycle indicate that the administration of Compound I leads to modulation of gene expression, resulting in a tumor microenvironment that is favorable for re-sensitizing tumors to other therapeutic agents including but not limited to CDK4/6 inhibitors, endocrine therapies such as SERDs, chemotherapeutic agents, immune checkpoint inhibitors, or a combination thereof. This improvement provides a significant advance in the state of the art of cancer treatment.

It is well understood that host's cells begin to evade CDK4/6 inhibition by activating and/or upregulating other cyclins and/or CDKs to promote uncontrolled cellular division once again. Of CDK 4/6 inhibitor trials that have led to FDA approval, at least 33% of patients developed recurrent disease on CDK4/6 inhibitors within only 2 years. In the PALOMA-2 trial, over 70% of subjects administered palbociclib in combination with letrozole for the treatment of advanced breast cancer developed disease progression over 3 years following initial therapy start (Finn et al. Lancet Oncol. 16:25-35(2016)).

The percent of patients with advanced ER+ breast cancer having deletion or mutation of the retinoblastoma (Rb) gene is extremely rare (3.9%) (Ciriello et al. Cell. 163:506-19(2015)), suggesting that an intact CDK/Rb axis is necessary for a patient to respond to CDK4/6 inhibition. Indeed, patients with advanced ER+ breast cancer are selected for CDK4/6 inhibitor treatment on the basis of the cancer expressing Rb. Studies in pre-clinical models demonstrate that low or absent Rb expression renders the tumor unresponsive to CDK4/6 inhibition (Konecny et al. Clin Canc Res. 17:1591-1602(2011); Thangavel et al. Endocr Relat Canc. 18:333-45(2011)). For example, in a study of 13 ex vivo tumor explant breast cancer models administered palbociclib, the two samples which exhibited no response lacked Rb expression (Dean et al. Cell Cycle. 11:2756-61(2012)). Similar studies of pancreatic cancer and glioblastoma cancer models demonstrate similar results linking the efficacy of CDK4/6 inhibition to an intact CDK/Rb axis (Michaud et al. Canc Res. 70:3228-38(2010); Chou et al. Gut. 67(12): 2142-55(2017)).

CDK2 plays a crucial role in promoting G1/S transition and S phase progression. In complex with cyclin E, CDK2 phosphorylates retinoblastoma pocket protein family members (p107, p130, pRb), leading to de-repression of E2F transcription factors, expression of G1/S transition related genes, and transition from G1 to S phase. This in turn enables activation of CDK2/cyclin A, which phosphorylates endogenous substrates that permit DNA synthesis, replication, and centrosome duplication. Another crucial adaptation cancer cells develop is the activation or overexpression of cyclin E (CCNE1 or CCNE2). Increased CCNE1/2 levels or activity subverts the cell cycle arrest induced by CDK4/6 inhibitors by activating alternative CDKs (Taylor-Harding et al. (2015); Herrera-Abreu et al. (2016); Bollard et al. (2017); Martin et al. (2017); Yang et al. (2017)). Dysregulated CDK2 activity commonly occurs through amplification of CCNE1 (gene that encodes cyclin E1 protein) and/or overexpression of cyclin E1 and mutations that inactivate CDK2 endogenous inhibitors (e.g., p27), respectively. In certain embodiments Compound I is used to treat the subset of cancers that has increased CCNE1/2 levels or activity.

### I. Terminology

Unless defined otherwise, all technical and scientific terms used herein have the same meaning as is commonly understood by one of skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Each of the references cited herein are incorporated by reference in its entirety.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

In certain embodiments, the term “about” means ±10%.

The “patient” or “subject” or “participant” treated is typically a human patient, unless otherwise indicated. In alternative embodiments, the methods described herein can be used to treat or in testing or other animals that respond similarly, such as mammals, for example, such as those used in preclinical testing including but not limited to mice, rats, monkeys, dogs, pigs, and rabbits; as well as domesticated swine (pigs and hogs), ruminants, equine, poultry, felines, bovines, murines, canines, and the like.

An “effective amount” as used herein, means an amount which provides a therapeutic benefit.

To “treat” a disease as the term is used herein, means to reduce the frequency or severity of at least one sign or symptom of a disease or disorder experienced by a patient (i.e., palliative treatment) or to decrease a cause or effect of the disease, disorder (i.e., disease-modifying treatment), or side effect experienced by a patient as a result of the administration of a therapeutic agent.

The term “treatment-emergent adverse events” as used herein, means an adverse event that occurs or worsens after the first dose or during treatment.

The term “overall survival (OS)” as used herein, means the time from the first dosing until death due to any causes.

The term “overall response rate (ORR)” as used herein, means the occurrence of either a confirmed best overall response of CR or PR, as determined by the RECIST v1.1 criteria.

The term “disease control rate (DCR)” as used herein, means the occurrence of a confirmed best overall response of CR, PR, or SD, as determined by the RECIST v1.1 criteria.

The term “progression free survival (PFS)” as used herein, means the duration of time from the first dosing until the first documented disease progression or death of any causes, whichever occurs first.

The term “duration of response (DOR)” as used herein, means the time from when confirmed best overall response of CR or PR was first documented to the first documented disease progression or death of any causes, whichever occurs first.

The term “time to progression (TTP)” as used herein, means the time from the first dosing until the first documented disease progression.

The term “MTD”, as used herein, refers to maximum tolerated dose of Compound I.

The term “AUC”, as used herein, refers to area under the concentration-time curve of Compound I.

The term “AUC₀₋ₜ”, as used herein, refers to AUC to time of last measurable concentration of Compound I.

The term “AUC_{0-∞}”, as used herein, refers to AUC to time infinity of Compound I.

The term “Cl”, as used herein, refers to clearance of Compound I of Compound I.

The term “Cₘₐₓ”, as used herein, refers to maximum concentration of Compound I.

The term “IC₅₀”, as used herein, refers to the half-maximal inhibitory concentration of Compound I. IC₅₀, a measure of the potency of a drug, indicates the concentration of Compound I that inhibits a selected biological process by half compared to a control compound.

The term “nH”, as used herein, refers to the Hill slope factor that describes the slope of the sigmoidal curve fitted to IC₅₀ data between min and max plateaus.

The term “t_{1/2}”, as used herein, refers to half-life of Compound I.

The term “t_{1/2α}”, as used herein, refers to initial elimination half-life of Compound I.

The term “t_{1/2β}”, as used herein, refers to terminal elimination half-life of Compound I.

The term “tₘₐₓ”, as used herein, refers to time to reach Cₘₐₓ of Compound I.

The term “tₗₐₛₜ”, as used herein, refers to time to reach last measurable concentration of Compound I.

The term “v_{d}”, as used herein, refers to volume of distribution of Compound I.

“Intrinsic resistance,” also known as primary resistance, as used herein, refers to a condition wherein a cancer is not responsive to, or sufficiently responsive to, the inhibitory effects of initial anti-cancer treatment, for example, but not limited to CDK4/6 inhibitor treatment or endocrine therapy treatment. In some embodiments of the methods of treatment described herein, the cancer treated is intrinsically resistant to a CDK4/6 inhibitor. Mutations and conditions associated with, for example, CDK4/6 inhibitor intrinsic resistance include, but are not limited to: increased activity of cyclin-dependent kinase 1 (CDK1); increased activity of cyclin-dependent kinase 2 (CDK2); loss, deficiency, or absence of retinoblastoma tumor suppressor protein (Rb) (Rb-null); high levels of p16Ink4a expression; high levels of MYC expression; increased expression of cyclin E1, cyclin E2, and cyclin A; and combinations thereof. The CDK4/6 inhibitor intrinsically resistant cancer may be characterized by reduced expression of the retinoblastoma tumor suppressor protein or a retinoblastoma family member protein or proteins (such as, but not limited to p107 and p130). In certain embodiments, a tumor or cancer that is intrinsically resistant to selective CDK4/6 inhibitor inhibition is a tumor or cancer whose cell population, as a whole, does not experience substantial G1 cell-cycle arrest when exposed to a selective CDK4/6 inhibitor. In certain embodiments, a tumor or cancer that is intrinsically resistant to CDK4/6 inhibitor inhibition is a tumor or cancer who has a cell population wherein less than 25%, 20%, 15%, 10%, or 5% of its cells experience G1 cell-cycle arrest when exposed to a selective CDK4/6 inhibitor. In some alternative embodiments of the methods of treatment described herein, the cancer treated is intrinsically resistant to an endocrine therapy, for example an estrogen inhibitor therapy such as a SERD. In some alternative embodiments of the methods of treatment described herein, the cancer treated is intrinsically resistant to a bioactive agent or other anti-cancer therapy.

“Acquired resistance,” as used herein, refers to a condition wherein a cancer that was or is initially sensitive to the inhibitory effects of an anti-cancer therapy becomes non-responsive or less-responsive over time to the effects of the administration of that therapy. In some embodiments of the methods described herein, the cancer to be treated has acquired resistance to a selective CDK4/6 inhibitor. Without wishing to be bound by any one theory, it is believed that acquired resistance to CDK4/6 inhibitors occurs due to one or more additional mutations or genetic alterations in bypass signaling that develops after the onset of CDK 4/6 inhibitor treatment regimen. For example, non-limiting exemplary causes of acquired resistance to CDK4/6 inhibitors may be a result of: the development of one or more genetic aberrations associated with “intrinsic resistance”. In addition, other non-limiting exemplary causes of acquired resistance to CDK4/6 inhibitors may include an increase in cyclin E expression; CCNE1/2 amplification; E2F amplification; CDK2 amplification; amplification of CDK6; amplification of CDK4; p16 amplification; WEE1 overexpression; MDM2 overexpression; CDK7 overexpression; loss of FZR1; HDAC activation; activation of the FGFR pathway; activation of the PI3K/AKT/mTOR pathway; loss of ER or PR expression; higher transcriptional activity of AP-1; epithelial-mesenchymal transition; Smad 3 suppression; autophagy activation; Rb1-loss or inactivating RB1 mutations; or a combination thereof A general review of CDK4/6 resistant mechanisms can be found, for example, in Pandey et al., Molecular mechanisms of resistance to CDK 4/6 inhibitors in breast cancer: A review. Int. J. Cancer: 00,1-10(2019), incorporated herein by reference. In certain embodiments, a tumor or cancer that has acquired resistance to selective CDK4/6 inhibitor inhibition is a tumor or cancer whose cell population, as a whole, no longer experiences substantial G1 cell-cycle arrest when exposed to a selective CDK4/6 inhibitor, resulting in disease progression. In certain embodiments, a tumor or cancer that has acquired resistance to CDK4/6 inhibitor inhibition is a tumor or cancer who has a cell population wherein less than 50%, 40%, 30% 20%, 15%, 10%, or 5% of its cells experience G1 cell-cycle arrest when exposed to a selective CDK4/6 inhibitor, leading to disease progression. In some embodiments, the cancer has progressed following a prior therapeutic regimen comprising the administration of a CDK4/6 inhibitor. In some alternative embodiments of the methods of treatment described herein, the cancer treated has acquired resistance, for example to an estrogen inhibitor therapy such as a SERD, for example, but not limited to, fulvestrant or elacestrant. In some alternative embodiments of the methods of treatment described herein, the cancer treated has acquired resistance to a bioactive agent or other anti-cancer therapy.

### II. Methods of Treatment

In certain aspects, methods described herein are provided for treating a proliferative disorder mediated by overexpression or amplification of cyclin E in a subject, including a human, is provided comprising administering an effective amount of Compound I or its pharmaceutically acceptable salt, deuterated derivative, or morphic form described herein and/or a pharmaceutically acceptable composition thereof. Non-limiting examples of disorders mediated by overexpression or amplification of cyclin E include tumors and cancers mediated by overexpression or amplification of cyclin E.

In other aspects, methods described herein are provided for treating a proliferative disorder mediated by CDK2 in a subject, including a human, is provided comprising administering an effective amount of a morphic form of Compound I as described herein; a pharmaceutical composition comprising a morphic form of Compound I; or a pharmaceutical composition manufactured from a morphic form of Compound I; or its pharmaceutically acceptable salt, deuterated derivative. Non-limiting examples of disorders mediated by CDK2 include tumors, cancers, disorders related to abnormal cellular proliferation, inflammatory disorders, immune disorders, and autoimmune disorders.

In another aspect, methods described herein are provided for treating a subject with a CDK4/6-resistant cancer comprising administering to the subject an effective amount of Compound I, or a pharmaceutically acceptable salt thereof or morphic form described herein; and, administering to the subject an effective amount of a CDK4/6 inhibitor. In certain embodiments, the CDK4/6 inhibitor resistant cancer is intrinsically resistant to a CDK4/6 inhibitor. In certain embodiments, the CDK4/6 inhibitor resistant cancer has acquired resistance to a CDK4/6 inhibitor. In certain embodiments, the CDK4/6-inhibitor resistant cancer has progressed following a prior regimen comprising a CDK4/6 inhibitor. In some embodiments, the CDK 4/6 inhibitor of the prior regimen is selected from palbociclib, ribociclib, abemaciclib, trilaciclib, lerociclib, SHR6390 (dalpiciclib), or a combination thereof. In some embodiments, the CDK4/6 inhibitor of the prior regimen is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib.

In certain embodiments the method of treatment comprises administering a pharmaceutical composition manufactured from a morphic form described herein. In other embodiments the method of treatment comprises administering a morphic form described herein.

In certain aspects, disclosed herein is a method for treating a subject with a CDK4/6 inhibitor-resistant small cell lung cancer (SCLC) comprising administering to the subject an effective amount of Compound I, or a pharmaceutically acceptable salt thereof, or a morphic form as described herein. In certain embodiments, the method further comprises administering an effective amount of a CDK4/6 inhibitor. In some embodiments, the CDK4/6 inhibitor is selected from palbociclib, ribociclib, abemaciclib, trilaciclib, lerociclib, SHR6390 (dalpiciclib). In some embodiments, the CDK 4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In certain embodiments, the method further comprises administering an effective amount of an additional anti-cancer therapy. In some embodiments, the anti-cancer therapy is selected from a chemotherapeutic agent, radiation, surgery, immune checkpoint inhibitor, estrogen inhibitor, androgen inhibitor, PARP inhibitor, or a combination thereof. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof.

In certain aspects, disclosed herein are methods for treating a subject with an abnormal cellular proliferation, comprising monitoring a sample of the subject for overexpression and/or activation of cyclin E1 (CCNE1) and/or cyclin E2 (CCNE2) compared with a control sample, wherein overexpression and/or activation of CCNE1 and/or CCNE2 comprise a cyclin E amplified or overexpressed abnormal cellular proliferative disorder; and, upon determining a subject has a cyclin E amplified or overexpressed abnormal cellular proliferative disorder, administering to the subject an effective amount of Compound I, or a pharmaceutically acceptable salt thereof. In certain embodiments, cyclin E1 (CCNE1) and/or cyclin E2 (CCNE2) is overexpressed and/or activated in a sample of a subject to be treated by at least 1.5-fold, at least 2.0-fold, at least 2.5-fold, at least 3.0-fold, at least 3.5-fold, at least 4.0-fold, at least 4.5-fold, at least 5.0-fold, or greater than 5.0-fold compared with a control sample. In certain embodiments, the method further comprises administering an effective amount of an additional CDK4/6 inhibitor. In some embodiments, the additional CDK4/6 inhibitor is selected from palbociclib, ribociclib, abemaciclib, trilaciclib, lerociclib, or SHR6390 (dalpiciclib). In some embodiments, the additional CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, an NGS panel test is used to confirm CCNE1 or CCNE2 overexpression or amplification status. In some embodiments, an NGS panel test to confirm CCNE1 or CCNE2 overexpression or amplification status is selected from Foundation One® CDx, Foundation One® Liquid CDx, Tempus xT (solid tumor), Tempus XF (liquid biopsy), Caris® Life Sciences Molecular Profiling, or OncoHelix Solid Tumor NGS. In some embodiments, the patient sample is selected from tumor tissue, formalin-fixed paraffin embedded (FFPE) tumor tissue, blood, or blood plasma.

In a principal embodiment the cancer treated by Compound I or a pharmaceutically acceptable salt thereof or morphic form described herein is a cyclin E amplified or overexpressed cancer or small cell lung cancer.

In some embodiments, the cyclin E amplified or overexpressed abnormal cellular proliferative disorder to be treated according to the detailed methods described above is selected from uterine cancer, uterine carcinosarcoma (UCS), uterine corpus endometrial carcinoma (UCEC), ovarian cancer, ovarian serous cystadenocarcinoma (OV), sarcoma (SARC), lung cancer, lung squamous cell carcinoma (LUSC), lung adenocarcinoma (LUAD), stomach cancer, stomach adenocarcinoma (STAD), bladder cancer, bladder urothelial carcinoma (BLCA), esophageal cancer, esophageal carcinoma (ESCA), adrenocortical carcinoma, breast cancer, breast invasive carcinoma (BRCA), pancreatic cancer, pancreatic adenocarcinoma (PAAD), fallopian tube cancer, primary peritoneal cancer, liver cancer, liver hepatocellular carcinoma (LIHC), cervical cancer, cervical squamous cell carcinoma (CESC), endocervical adenocarcinoma, mesothelioma (MESO), head and neck squamous cell carcinoma (HSNC), colon cancer, colon adenocarcinoma (COAD), skin cancer, melanoma, skin cutaneous melanoma (SKCM), glioblastoma multiforme (GBM), kidney cancer, or kidney chromophobe (KICH). In some embodiments, the cyclin E amplified or overexpressed cancer is retinoblastoma (Rb) protein positive. In some embodiments, the cyclin E amplified or overexpressed cancer is CDK4/6 inhibitor-resistant. In certain embodiments, the CDK4/6 inhibitor resistant cancer is intrinsically resistant to a CDK4/6 inhibitor. In certain embodiments, the CDK4/6 inhibitor resistant cancer has acquired resistance to a CDK4/6 inhibitor. In some embodiments, the cyclin E amplified or overexpressed cancer is endocrine therapy treatment-resistant. In certain embodiments, the endocrine therapy treatment resistant cancer is intrinsically resistant to an endocrine therapy. In certain embodiments, the endocrine therapy treatment resistant cancer has acquired resistance to an endocrine therapy. In some embodiments, the endocrine therapy comprises an estrogen inhibitor as described herein. In certain embodiments, the cancer is advanced and/or metastatic cancer. In certain embodiments, the cancer is an unresectable cancer. In certain embodiments, the cancer is advanced unresectable cancer. In certain embodiments, the cancer is platinum-refractory and/or platinum-resistant. In certain embodiments, the cancer has progressed following a prior standard of care regimen. In certain embodiments, the cancer has progressed following a prior standard systemic therapy. In certain embodiments, the cancer has progressed following a prior systemic anti-cancer therapy. In certain embodiments, the cancer has progressed following a prior regimen comprising a platinum analog. In certain embodiments, the cancer has progressed following a prior regimen comprising a CDK4/6 inhibitor. In certain embodiments, the cancer has progressed following a prior regimen comprising an estrogen inhibitor.

In certain embodiments, the methods described herein are useful for the treatment of a cyclin E-overexpressed and/or amplified ovarian cancer, wherein the ovarian cancer has an amplification of CCNE1. In certain embodiments, the ovarian cancer is an advanced and/or metastatic cancer. In certain embodiments, the ovarian cancer is advanced unresectable cancer. In certain embodiments, the ovarian cancer is platinum-refractory and/or platinum-resistant. In certain embodiments, the ovarian cancer has progressed following a prior standard of care regimen. In certain embodiments, the ovarian cancer has progressed following a prior standard systemic therapy. In certain embodiments, the ovarian cancer has progressed following a prior systemic anti-cancer therapy. In certain embodiments, the ovarian cancer has progressed following a prior regimen comprising a platinum analog. In certain embodiments, the ovarian cancer has progressed following a prior regimen comprising a CDK4/6 inhibitor. In certain embodiments, the ovarian cancer has progressed following a prior regimen comprising an estrogen inhibitor.

In certain embodiments, the methods as described fully herein further comprise administering an effective amount of an additional anti-cancer therapy. In some embodiments, the anti-cancer therapy is selected from radiation, surgery, immune checkpoint inhibitor, estrogen inhibitor, androgen inhibitor, PARP inhibitor, or a combination thereof. In some embodiments, the cyclin E amplified or overexpressed cancer is uterine cancer. In some embodiments, the cyclin E amplified or overexpressed cancer is ovarian cancer. In some embodiments, the cyclin E amplified or overexpressed cancer is breast cancer. In certain embodiments, the method further comprises administering an effective amount of an estrogen inhibitor. In some embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In some embodiments, the estrogen inhibitor is a selective estrogen receptor degrader (SERD). In some embodiments, the SERD comprises fulvestrant. In some embodiments, the SERD comprises elacestrant (RAD1901). In some embodiments, the cyclin E amplified or overexpressed cancer is prostate cancer. In some embodiments, the cyclin E amplified or overexpressed cancer is bladder cancer. In some embodiments, the cyclin E amplified or overexpressed cancer is sarcoma.

Compound I or a pharmaceutically acceptable salt thereof or morphic form described herein is useful as a therapeutic agent when administered in an effective amount to a subject, including a human, to treat a tumor, cancer (solid, non-solid, diffuse, hematological, etc.), abnormal cellular proliferation, immune disorder, inflammatory disorder, blood disorder, a myelo- or lymphoproliferative disorder such as B- or T-cell lymphomas, multiple myeloma, breast cancer, prostate cancer, AML, ALL, CLL, myelodysplastic syndrome (MDS), mesothelioma, renal cell carcinoma (RCC), cholangiocarcinoma, lung cancer, pancreatic cancer, colon cancer, skin cancer, melanoma, Waldenstrom's macroglobulinemia, Wiskott-Aldrich syndrome, or a post-transplant lymphoproliferative disorder; an autoimmune disorder, for example, Lupus, Crohn's Disease, Addison disease, Celiac disease, dermatomyositis, Graves disease, thyroiditis, multiple sclerosis, pernicious anemia, reactive arthritis, or type I diabetes; a disease of cardiologic malfunction, including hypercholesterolemia; an infectious disease, including a viral and/or bacterial infection; an inflammatory condition, including asthma, chronic peptic ulcers, tuberculosis, rheumatoid arthritis, periodontitis, ulcerative colitis, or hepatitis.

In certain embodiments, Compound I or a pharmaceutically acceptable salt thereof or morphic form described herein is used to treat a cyclin E overexpressed or amplified breast cancer. In certain embodiments, the breast cancer is HR+ and HER2−. In certain embodiments, the breast cancer is HR− and HER2+. In certain embodiments, the breast cancer is ER+ and HER2−. In certain embodiments, the breast cancer is ER− and HER2+. In certain embodiments, the breast cancer is HER2 overexpressing breast cancer. In certain embodiments, the breast cancer comprises HER2 gene amplification. In some embodiments, Compound I or a pharmaceutically acceptable salt thereof is administered in combination with an antibody drug conjugate (ADC). In some embodiments, Compound I or a pharmaceutically acceptable salt thereof is administered in combination with an ADC selected from ado-trastuzumab emtansine (KADCYLA®), trastuzumab deruxtecan (ENHERTU®), or sacituzumab govetican (TRODELVY®).

In certain embodiments, the Compound I or a pharmaceutically acceptable salt thereof or morphic form described herein is used to treat a cyclin E overexpressed or amplified non-small cell lung cancer (NSCLC). In certain embodiments, the NSCLC has an EGFR mutation. In certain embodiments, the NSCLC has an EGFR mutation and an EGFR inhibitor failed (e.g. 2^{nd} line therapy). In certain embodiments, an ALK inhibitor failed (e.g. 2^{nd} line therapy). In certain embodiments, the NSCLC has an KRAS mutation.

In certain embodiments, the Compound I or a pharmaceutically acceptable salt thereof or morphic form described herein is used to treat a cyclin E overexpressed or amplified prostate cancer. In certain embodiments, the prostate cancer is castration resistant. In certain embodiments, a prior chemotherapeutic agent already failed (e.g. 2^{nd} line therapy).

In certain embodiments, the Compound I or a pharmaceutically acceptable salt thereof or morphic form described herein is used to treat a cyclin E overexpressed or amplified lymphoma. In certain embodiments, the lymphoma is mantel cell lymphoma (MCL), marginal zone lymphoma (MZL), chronic lymphocytic leukemia (CLL), follicular lymphoma (FL), or diffuse large B-cell lymphoma (DLBCL). In certain embodiments, a prior chemotherapeutic agent already failed (e.g. 2^{nd} line therapy).

In certain embodiments, the Compound I or a pharmaceutically acceptable salt thereof or morphic form described herein is used to treat a cyclin E overexpressed or amplified melanoma. In certain embodiments, the melanoma has a BRAF mutation.

In certain embodiments, the Compound I or a pharmaceutically acceptable salt thereof or morphic form described herein is used to treat a cyclin E overexpressed or amplified RAS mutated cancer. In certain embodiments, the RAS mutated cancer is colon cancer (CLC). In certain embodiments, the RAS mutated cancer is pancreatic cancer. In certain embodiments the RAS mutated cancer is cholangiocarcinoma.

In certain embodiments, the Compound I or a pharmaceutically acceptable salt thereof or morphic form described herein is used to treat a cyclin E overexpressed or amplified gastrointestinal stromal tumor (GIST). In certain embodiments, the treatment with imatinib or sunitinib already failed (e.g. 2^{nd} line therapy).

Exemplary proliferative disorders to be treated by the detailed methods, compounds, and morphic forms described herein include, but are not limited to, benign growths, neoplasms, tumors, cancer (Rb positive or Rb negative), autoimmune disorders, inflammatory disorders graft-versus-host rejection, and fibrotic disorders wherein the disorder is mediated by overexpressed or amplified cyclin E.

Non-limiting examples of cancers that can be treated according to the detailed methods, compounds, and morphic forms described herein include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva). In some embodiments, the cancer has overexpression and/or amplification of CCNE1 and/or CCNE2. In some embodiments, the cancer is CDK4/6 inhibitor-resistant. In some embodiments, the CDK4/6 inhibitor-resistant cancer is retinoblastoma (Rb) protein-positive (Rb+). In some embodiments, the CDK4/6 inhibitor-resistant cancer is retinoblastoma (Rb) protein-null (Rb−). In some embodiments, the cancer is resistant to endocrine therapy. In some embodiments, the endocrine therapy comprises an estrogen inhibitor.

In another embodiment, the methods, compounds, and morphic forms described herein are useful for the treatment of myelodysplastic syndrome (MDS).

In certain embodiments, the methods, compounds, and morphic forms described herein are useful for the treatment of a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the methods, compounds, and morphic forms described herein are useful for the treatment of a myeloproliferative neoplasm. In certain embodiments, the myeloproliferative neoplasm (MPN) is primary myelofibrosis (PMF).

In certain embodiments, the methods, compounds, and morphic forms described herein are useful for the treatment of a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

In certain embodiments, the condition treated with Compound I or a pharmaceutically acceptable salt thereof or morphic form described herein, is a disorder related to abnormal cellular proliferation. Abnormal cellular proliferation, notably hyperproliferation, can occur as a result of a wide variety of factors, including genetic mutation, infection, exposure to toxins, autoimmune disorders, and benign or malignant tumor induction.

The methods, compounds, and morphic forms described herein are useful for the treatment of a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

The methods, compounds, and morphic forms described herein are useful for the treatment of other hyperproliferative cell disorders which include blood vessel proliferative disorders, fibrotic disorders, autoimmune disorders, graft-versus-host rejection, tumors and cancers.

The methods, compounds, and morphic forms described herein are useful for the treatment of blood vessel proliferative disorders which include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

The methods, compounds, and morphic forms described herein are useful for the treatment of fibrotic disorders which are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

The methods, compounds, and morphic forms described herein are useful for the treatment of mesangial disorders, which are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

The methods, compounds, and morphic forms described herein are useful for the treatment of a rheumatoid arthritis, which is a disease with a proliferative component. Rheumatoid arthritis is generally considered an autoimmune disease that is thought to be associated with activity of autoreactive T cells, and to be caused by autoantibodies produced against collagen and IgE.

The methods, compounds, and morphic forms described herein are useful for the treatment of other disorders that can include an abnormal cellular proliferative component selected from the group consisting of Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

In certain embodiments, the methods, compounds, and morphic forms described herein are useful for the treatment of a condition associated with an immune response.

For example, cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

In certain non-limiting embodiments, Compound I or a pharmaceutically acceptable salt thereof or morphic form described herein is used as a topical agent in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides. These compounds can also be used to treat an aqueous-deficient dry eye state (such as immune mediated keratoconjunctivitis) in a patient suffering therefrom, by administering the compound topically to the eye.

Exemplary cancers which may be treated by the present disclosed methods, compounds, and morphic forms either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, Ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+ DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In another aspect, a method of increasing BIM expression (e.g., BCLC2L11 expression) is provided to induce apoptosis in a cell comprising contacting Compound I or a pharmaceutically acceptable composition, salt, morphic form described herein, or isotopic analog thereof with the cell. In certain embodiments, the method is an in vitro method. In certain embodiments, the method is an in vivo method. BCL2L11 expression is tightly regulated in a cell. BCL2L11 encodes for BIM, a proapoptotic protein. BCL2L11 is downregulated in many cancers and BIM is inhibited in many cancers, including chronic myelocytic leukemia (CML) and non-small cell lung cancer (NSCLC) and that suppression of BCL2L11 expression can confer resistance to tyrosine kinase inhibitors. See, e.g., Ng et al., Nat. Med. (2012) 18:521-528.

In yet another aspect, methods described herein are provided for treating a condition associated with angiogenesis is provided. For example, provided herein is a method of treating a condition associated with angiogenesis selected from a diabetic condition (e.g., diabetic retinopathy), an inflammatory condition (e.g., rheumatoid arthritis), macular degeneration, obesity, atherosclerosis, or a proliferative disorder, comprising administering to a subject in need thereof Compound I or a pharmaceutically acceptable composition, salt, morphic form described herein or isotopic analog thereof.

In certain embodiments, the condition associated with angiogenesis is macular degeneration. In certain embodiments, provided is a method of treating macular degeneration comprising administering to a subject in need thereof Compound I or a pharmaceutically acceptable composition, salt, morphic form as described herein, or isotopic analog thereof.

In certain embodiments, the condition associated with angiogenesis is obesity. As used herein, “obesity” and “obese” as used herein, refers to class I obesity, class II obesity, class III obesity and pre-obesity (e.g., being “over-weight”) as defined by the World Health Organization. In certain embodiments, a method of treating obesity is provided comprising administering to a subject in need thereof Compound I or a pharmaceutically acceptable composition, salt, morphic form as described herein, or isotopic analog thereof.

In certain embodiments, the condition associated with angiogenesis is atherosclerosis. In certain embodiments, provided is a method of treating atherosclerosis comprising administering to a subject in need thereof Compound I or a pharmaceutically acceptable composition, salt, morphic form as described herein, or isotopic analog thereof.

In certain embodiments, the condition associated with angiogenesis is a proliferative disorder. In certain embodiments, provided is a method of treating a proliferative disorder comprising administering to a subject in need thereof Compound I or a pharmaceutically acceptable composition, salt, morphic form as described herein, or isotopic analog thereof.

The present invention provides advantageous methods to treat a subject with a cyclin E amplified or overexpressed cancer which includes monitoring a sample of the subject for overexpression and/or activation of cyclin E1 (CCNE1) and/or cyclin E2 (CCNE2) compared with a control sample, wherein overexpression and/or activation of CCNE1 and/or CCNE2 comprise a cyclin E amplified or overexpressed abnormal cellular proliferative disorder, and upon determining a subject has a cyclin E amplified or overexpressed abnormal cellular proliferative disorder, administering an effective amount of Compound I, or a pharmaceutically acceptable composition, salt, morphic form as described herein, or isotopic analog thereof. In certain aspects, Compound I or a pharmaceutically acceptable salt thereof, is used to treat a subject with a cyclin E amplified or overexpressed cancer. In certain aspects, Compound I or a pharmaceutically acceptable salt thereof, or morphic form described herein, is used to treat a subject screened and determined to have an elevated level of cyclin E expression when compared with a control subject. In certain aspects, Compound I or a pharmaceutically acceptable salt thereof, or morphic form described herein is used to treat a subject screened and determined to have an elevated level of cyclin E activation when compared with a control subject. In some embodiments, an NGS panel test is used to confirm CCNE1 or CCNE2 overexpression or amplification status. In some embodiments, an NGS panel test to confirm CCNE1 or CCNE2 overexpression or amplification status is selected from Foundation One® CDx, Foundation One® Liquid CDx, Tempus xT (solid tumor), Tempus xF (liquid biopsy), Caris® Life Sciences Molecular Profiling, or OncoHelix Solid Tumor NGS. In some embodiments, the patient sample is selected from tumor tissue, formalin-fixed paraffin embedded (FFPE) tumor tissue, blood, or blood plasma. In certain embodiments, cyclin E1 (CCNE1) and/or cyclin E2 (CCNE2) is overexpressed and/or activated in a sample of a subject to be treated by at least 1.5-fold, at least 2.0-fold, at least 2.5-fold, at least 3.0-fold, at least 3.5-fold, at least 4.0-fold, at least 4.5-fold, at least 5.0-fold, or greater than 5.0-fold compared with a control sample. In certain aspects, Compound I or a pharmaceutically acceptable salt thereof or morphic form described herein, is used in combination with a CDK4/6 inhibitor to treat a subject with a cyclin E amplified or overexpressed cancer. In certain aspects, Compound I or a pharmaceutically acceptable salt thereof or morphic form described herein, is used to treat a subject with a cyclin E amplified or overexpressed cancer that is CDK4/6 inhibitor resistant. In certain embodiments, the CDK4/6 inhibitor resistant cancer is intrinsically resistant to a CDK4/6 inhibitor. In certain embodiments, the CDK4/6 inhibitor resistant cancer has acquired resistance to a CDK4/6 inhibitor. In certain aspects, Compound I or a pharmaceutically acceptable salt thereof or morphic form described herein, is used in combination with a CDK4/6 inhibitor to re-sensitize a subject with a cyclin E amplified or overexpressed cancer that is CDK4/6 inhibitor resistant to treatment. In some embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.

In another aspect, disclosed herein are methods for treating a subject with a CCNE1-amplified cancer. For example, disclosed herein is method for treating a subject with an advanced and/or metastatic solid tumor having a CCNE1 amplification, comprising administering to the subject an effective amount of Compound I, or a pharmaceutically acceptable salt thereof or morphic form as described herein. In certain embodiments, the advanced and/or metastatic solid tumor having a CCNE1 amplification is platinum-resistant. In certain embodiments, the advanced and/or metastatic solid tumor having a CCNE1 amplification is platinum-refractory. In certain embodiments, the solid tumor has progressed following a prior standard of care regimen. In certain embodiments, the advanced or metastatic solid tumor is intolerant to or is ineligible for standard therapy. In certain embodiments, the method further comprises administering an effective amount of a CDK4/6 inhibitor. In certain embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In certain embodiments, the method further comprises administering an effective amount of an additional anti-cancer therapy. In some embodiments, the anti-cancer therapy is selected from a chemotherapeutic agent, radiation, surgery, immune checkpoint inhibitor, estrogen inhibitor, androgen inhibitor, PARP inhibitor, or a combination thereof. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof. In certain embodiments, the anti-cancer therapy is an estrogen inhibitor. In certain embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In certain embodiments, the estrogen inhibitor is a selective estrogen receptor degrader (SERD). In certain embodiments, the SERD is selected from fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, or ZN-c5. In some embodiments, the SERD comprises fulvestrant. In some embodiments, the SERD comprises elacestrant (RAD1901).

In another aspect, disclosed herein are methods for treating a subject with a CDK4/6-resistant cancer comprising administering to the subject an effective amount of Compound I, or a pharmaceutically acceptable salt thereof or morphic form described herein; and, administering to the subject an effective amount of a CDK4/6 inhibitor. In certain embodiments, the CDK4/6 inhibitor resistant cancer has acquired resistance to a CDK4/6 inhibitor. In certain embodiments, the CDK4/6-inhibitor resistant cancer has progressed following a prior regimen comprising a CDK4/6 inhibitor. In some embodiments, the CDK4/6 inhibitor of the prior regimen is selected from palbociclib, ribociclib, abemaciclib, trilaciclib, lerociclib, SHR6390 (dalpiciclib), or a combination thereof. In some embodiments, the CDK4/6 inhibitor of the prior regimen is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In certain embodiments, the CDK4/6 inhibitor-resistant cancer is intrinsically resistant to CDK4/6 inhibitors. In some embodiments, the CDK4/6 inhibitor-resistant cancer is retinoblastoma (Rb) protein-positive (Rb+). In some embodiments, the CDK4/6-inhibitor resistance cancer has an intrinsic CDK4/6 inhibitor resistance. In some embodiments, the CDK4/6 inhibitor cancer is retinoblastoma (Rb) protein-null (Rb−). In some embodiments, the CDK4/6-inhibitor resistant cancer is selected from breast cancer, lung cancer, small cell lung cancer, uterine cancer, endometrial cancer, ovarian cancer, prostate cancer, bladder cancer, testicular cancer, glioblastoma, head and/or neck cancer, or prostate cancer. In some embodiments, the CDK4/6-inhibitor resistant cancer is breast cancer. In some embodiments, the CDK4/6-inhibitor resistant breast cancer is estrogen receptor-positive (ER+) breast cancer. In certain embodiments, the CDK4/6-inhibitor resistant cancer is hormone receptor positive (HR+) breast cancer. In some embodiments, the CDK4/6-inhibitor resistant cancer is small cell lung cancer (SCLC). In certain embodiments, the CDK4/6-inhibitor resistant cancer is cyclin E amplified or overexpressed. In some embodiments, Compound I or a pharmaceutically acceptable salt thereof or morphic form described herein is administered to the subject at least once daily, and wherein an effective amount of the CDK4/6 inhibitor is administered according to its prescribed label. In some embodiments, Compound I or a pharmaceutically acceptable salt thereof is administered to the subject at least twice daily, and wherein an effective amount of the CDK4/6 inhibitor is administered according to its prescribed label. In some embodiments, the CDK4/6 inhibitor resistant cancer is also resistant to endocrine therapy or estrogen inhibitor, for example a SERD.

Determining intrinsic resistance to selective CDK4/6 inhibitors, for example by determining the loss or absence of retinoblastoma (Rb) tumor suppressor protein (Rb-null), can be determined through any of the standard assays known to one of ordinary skill in the art. For example, Rb-status in a cancer can be determined by, for example but not limited to, Western Blot, ELISA (enzyme linked immunoadsorbent assay), IHC (immunohistochemistry), and FACS (fluorescent activated cell sorting). The selection of the assay will depend upon the tissue, cell line or surrogate tissue sample that is utilized e.g., for example Western Blot and ELISA may be used with any or all types of tissues, cell lines or surrogate tissues, whereas the IHC method would be more appropriate wherein the tissue utilized in the methods of described herein was a tumor biopsy. FACs analysis would be most applicable to samples that were single cell suspensions such as cell lines and isolated peripheral blood mononuclear cells. See for example, US20070212736 “Functional Immunohistochemical Cell Cycle Analysis as a Prognostic Indicator for Cancer”.

Alternatively, molecular genetic testing may be used for determination of retinoblastoma gene status. Molecular genetic testing for retinoblastoma includes the following as described in Lohmann and Gallie “Retinoblastoma. Gene Reviews” (2010) or Parsam et al. “A comprehensive, sensitive and economical approach for the detection of mutations in the RB1 gene in retinoblastoma” Journal of Genetics, 88(4), 517-527 (2009).

Increased activity or levels of cyclin E can be determined through any of the standard assays known to one of ordinary skill in the art, including but not limited to Next Generation Sequencing (NGS), Western Blot, ELISA (enzyme linked immunoadsorbent assay), IHC (immunohistochemistry), and FACS (fluorescent activated cell sorting). The selection of the assay will depend upon the tissue, cell line, or surrogate tissue sample that is utilized e.g., for example Western Blot and ELISA may be used with any or all types of tissues, cell lines, or surrogate tissues, whereas the IHC method would be more appropriate wherein the tissue utilized in the methods was a tumor biopsy. FACs analysis would be most applicable to samples that were single cell suspensions such as cell lines and isolated peripheral blood mononuclear cells.

In some embodiments, an NGS panel test is used to confirm CCNE1 or CCNE2 overexpression or amplification status. NGS panel tests of patient samples are known to persons skilled in the art. In some embodiments, an NGS panel test to confirm CCNE1 or CCNE2 overexpression or amplification status is selected from Foundation One® CDx, Foundation One® Liquid CDx, Tempus xT (solid tumor), Tempus XF (liquid biopsy), Caris® Life Sciences Molecular Profiling, or OncoHelix Solid Tumor NGS. In some embodiments, the patient sample is selected from tumor tissue, formalin-fixed paraffin embedded (FFPE) tumor tissue, blood, or blood plasma. For example, an NGS panel is an in vitro diagnostic device used for the detection of substitutions, insertions, deletion alterations (e.g., indels), and copy number alternations in a panel of selected genes using DNA isolated from sample (e.g., formalin-fixed paraffin embedded (FFPE) tumor tissue). Briefly, DNA is extracted from a sample obtained from a patient using a DNA extraction method. Whole-genome shotgun library construction and hybridization-based capture is conducted using a next generation sequencing platform (e.g., Illumina® HiSeq 4000) to sequence coding exons or intronic regions of the panel of selected genes at high uniform depth. Sequence data is then processed post-collection to detect genomic alterations including but not limited to base substitutions, indels, copy number alterations (e.g., amplification, homozygous gene deletion), genomic rearrangements (e.g., gene fusions), microsatellite instability (MSI), tumor mutational burden (TMB), and positive homologous recombination deficiency (HRD) status. In some embodiments, an NGS panel test confirms CCNE1 or CCNE2 overexpression or amplification status at any time during or after original diagnosis but prior to initial administration of Compound I.

Immunohistochemistry (IHC) and immunocytochemistry (ICC) are techniques employed to localize expression and are dependent on specific epitope-antibody interactions. IHC refers to the use of tissue sections, whereas ICC describes the use of cultured cells or cell suspensions. In both methods, positive staining is visualized using a molecular label, which can be fluorescent or chromogenic. Briefly, samples are fixed to preserve cellular integrity and then subjected to incubation with blocking reagents to prevent non-specific binding of the antibodies. Samples are subsequently incubated with primary and secondary antibodies, and the signal is visualized for microscopic analysis.

The western blot technique uses three elements to identify specific proteins from a complex mixture of proteins extracted from cells: separation by size, transfer to a solid support, and marking target protein using a proper primary and secondary antibody to visualize. The most common version of this method is immunoblotting. This technique is used to detect specific proteins in a given sample of tissue homogenate or extract. The sample of proteins is first electrophoresed by SDS-PAGE to separate the proteins based on molecular weight. The proteins are then transferred to a membrane where they are probed using antibodies specific to the target protein.

Genomic alterations and mRNA expression can be determined through fluorescence in situ hybridization (FISH), targeted sequencing, and microarray analysis. Commonly mutated genes, as well as differentially expressed and co-expressed genes can be identified.

Fluorescence in situ hybridization (FISH) is a cytogenic technique used for the detection and localization of RNA sequences within tissues or cells. It is particularly important for defining the spatial-temporal patterns of gene expression. FISH relies on fluorescent probes that bind to complementary sequences of the RNA of interest. A series of hybridization steps are performed to achieve signal amplification of the target of interest. This amplification is then viewed using a fluorescent microscope. This technique can be used on formalin-fixed paraffin embedded (FFPE) tissue, frozen tissues, fresh tissues, cells and circulating tumor cells.

Targeted RNA-sequencing (RNA-Seq) is a highly accurate method for selecting and sequencing specific transcripts of interest. It offers both quantitative and qualitative information. Targeted RNA-Seq can be achieved via either enrichment or amplicon-based approaches, both of which enable gene expression analysis in a focused set of genes of interest. Enrichment assays also provide the ability to detect both known and novel gene fusion partners in many sample types, including formalin-fixed paraffin-embedded (FFPE) tissue. RNA enrichment provides quantitative expression information as well as the detection of small variants and gene fusions.

In a microarray analysis, mRNA molecules are typically collected from both an experimental sample and a reference sample. For example, the reference sample could be collected from a healthy individual, and the experimental sample could be collected from an individual with a disease such as cancer. The two mRNA samples are then converted into complementary DNA (cDNA), and each sample is labeled with a fluorescent probe of a different color. The experimental cDNA sample may be labeled with a red fluorescent dye, whereas the reference cDNA may be labeled with a green, fluorescent dye. The two samples are then mixed together and allowed to hybridize to the microarray slide. Following hybridization, the microarray is scanned to measure the expression of each gene printed on the slide. If the expression of a particular gene is higher in the experimental sample than in the reference sample, then the corresponding spot on the microarray appears red. In contrast, if the expression in the experimental sample is lower than in the reference sample, then the spot appears green. Finally, if there is equal expression in the two samples, then the spot appears yellow. The data gathered through microarrays can be used to create gene expression profiles, which show simultaneous changes in the expression of many genes in response to a particular condition or treatment.

In another aspect, disclosed herein is a method for treating a subject with a CDK4/6 inhibitor-resistant small cell lung cancer (SCLC) comprising administering to the subject an effective amount of Compound I, or a pharmaceutically acceptable salt thereof, or morphic form as described herein. In certain embodiments, the method further comprises administering an effective amount of an additional anti-cancer therapy. In some embodiments, the anti-cancer therapy is selected from a chemotherapeutic agent, radiation, surgery, immune checkpoint inhibitor, estrogen inhibitor, androgen inhibitor, PARP inhibitor, or a combination thereof. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof. In some embodiments, the chemotherapeutic agent is carboplatin. In some embodiments, the chemotherapeutic agent is etoposide. In some embodiments, the chemotherapeutic agent is doxorubicin. In some embodiments, the chemotherapeutic agent is camptothecin. In some embodiments, the chemotherapeutic agent is cisplatin.

In another aspect, disclosed herein is a method for treating a subject with advanced and/or metastatic ovarian cancer having a CCNE1 amplification, comprising administering to the subject an effective amount of Compound I, or a pharmaceutically acceptable salt thereof or morphic form as described herein. In certain embodiments, the advanced and/or metastatic ovarian cancer having a CCNE1 amplification is platinum-resistant. In certain embodiments, the advanced and/or metastatic ovarian cancer having a CCNE1 amplification is platinum-refractory. In certain embodiments, the ovarian cancer is epithelial ovarian cancer. In certain embodiments, the ovarian cancer is fallopian tube cancer. In certain embodiments, the ovarian cancer is primary peritoneal cancer. In certain embodiments, the ovarian cancer has progressed following a prior standard of care regimen. In certain embodiments, the ovarian cancer has progressed following a prior standard systemic therapy. In certain embodiments, the ovarian cancer has progressed following a prior systemic anti-cancer therapy. In certain embodiments, the ovarian cancer has progressed following a prior regimen comprising a platinum analog. In certain embodiments, the method further comprises administering an effective amount of a CDK4/6 inhibitor. In certain embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In certain embodiments, the method further comprises administering an effective amount of an additional anti-cancer therapy. In some embodiments, the anti-cancer therapy is selected from a chemotherapeutic agent, radiation, surgery, immune checkpoint inhibitor, estrogen inhibitor, androgen inhibitor, PARP inhibitor, or a combination thereof. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof. In certain embodiments, the anti-cancer therapy is an estrogen inhibitor. In certain embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In certain embodiments, the estrogen inhibitor is a selective estrogen receptor degrader (SERD). In some embodiments, the SERD comprises fulvestrant. In some embodiments, the SERD comprises elacestrant (RAD1901).

In another aspect, disclosed herein is a method for treating a subject with advanced and/or metastatic human epidermal growth factor 2 negative (HER2−) breast cancer, comprising administering to the subject an effective amount of Compound I, or a pharmaceutically acceptable salt thereof or morphic form as described herein. In certain embodiments, the breast cancer is hormone receptor positive (HR+). In certain embodiments, the breast cancer is HR+/HER2− breast cancer. In certain embodiments, the breast cancer is estrogen receptor positive (ER+). In certain embodiments, the breast cancer is ER+/HER2− breast cancer. In certain embodiments, the advanced and/or metastatic human epidermal growth factor 2 negative (HER2−) breast cancer is platinum-resistant. In certain embodiments, the advanced and/or metastatic human epidermal growth factor 2 negative (HER2−) breast cancer is platinum-refractory. In certain embodiments, the advanced and/or metastatic human epidermal growth factor 2 negative (HER2−) breast cancer has progressed following a prior standard of care regimen. In certain embodiments, the method further comprises administering an effective amount of an additional anti-cancer therapy. In some embodiments, the anti-cancer therapy is selected from a chemotherapeutic agent, radiation, surgery, immune checkpoint inhibitor, estrogen inhibitor, androgen inhibitor, PARP inhibitor, or a combination thereof. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof.

In certain embodiments, Compound I is administered at a dose of about 0.1 mg to about 2000 mg, from about 10 mg to about 1000 mg, from about 100 mg to about 800 mg, or from about 200 mg to about 600 mg, or a pharmaceutically acceptable salt thereof or morphic form as described herein. In certain embodiments, Compound I is administered at a dose from about 100 mg to about 800 mg. In certain embodiments, Compound I, or a pharmaceutically acceptable salt thereof or morphic form described herein, is a administered at a dose of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg. In certain embodiments, Compound I, or a pharmaceutically acceptable salt thereof or morphic form as described herein, is administered at a dose of about 100 mg. In certain embodiments, Compound I, or a pharmaceutically acceptable salt thereof or morphic form as described herein, is administered at a dose of about 200 mg. In certain embodiments, Compound I, or a pharmaceutically acceptable salt thereof or morphic form as described herein, is administered at a dose of about 300 mg. In certain embodiments, Compound I, or a pharmaceutically acceptable salt thereof or morphic form as described herein, is administered at a dose of about 400 mg. In certain embodiments, Compound I, or a pharmaceutically acceptable salt thereof or morphic form as described herein, is administered at a dose of about 500 mg. In certain embodiments, Compound I, or a pharmaceutically acceptable salt thereof or morphic form as described herein, is administered at a dose of about 600 mg. In certain embodiments, Compound I, or a pharmaceutically acceptable salt thereof or morphic form as described herein, is administered at a dose of about 700 mg. In certain embodiments, Compound I, or a pharmaceutically acceptable salt thereof or morphic form as described herein, is administered at a dose of about 800 mg.

In certain embodiments, the methods as described herein further comprises the administration of an effective amount of an additional anti-cancer therapy. In some embodiments, the anti-cancer therapy is selected from a chemotherapeutic agent, radiation, surgery, immune checkpoint inhibitor, estrogen inhibitor, androgen inhibitor, PARP inhibitor, or a combination thereof. In some embodiments, the anti-cancer therapy comprises a chemotherapeutic agent. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof. In some embodiments, the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof. In some embodiments, Compound I or a pharmaceutically acceptable salt thereof or morphic form as described herein is administered within 24 hours or less to the administration of the chemotherapeutic agent. In some embodiments, Compound I or a pharmaceutically acceptable salt thereof or morphic form as described herein is administered within 6 hours or less to the administration of the chemotherapeutic agent. In some embodiments, Compound I or a pharmaceutically acceptable salt thereof or morphic form as described herein is administered within 3 hours or less of the administration of the chemotherapeutic agent. In some embodiments, Compound I or a pharmaceutically acceptable salt thereof or morphic form as described herein is administered to the subject at least once daily, and wherein an effective amount of the anti-cancer therapy is administered according to its prescribed label. In some embodiments, Compound I or a pharmaceutically acceptable salt thereof or morphic form as described herein is administered to the subject at least twice daily, and wherein an effective amount of the anti-cancer therapy is administered according to its prescribed label.

In certain embodiments, the methods as described herein further comprise administering an effective amount of an estrogen inhibitor. In some embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In some embodiments, the estrogen inhibitor is a selective estrogen receptor degrader (SERD). In some embodiments, the SERD comprises fulvestrant. In some embodiments, the SERD comprises elacestrant (RAD1901).

In certain embodiments, the methods as described herein further comprise administering an effective amount of a chemotherapeutic agent. In some embodiments, the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof. In some embodiments, the CDK4/6 inhibitor-resistant ER+ breast cancer is luminal A breast cancer. In some embodiments, Compound I or a pharmaceutically acceptable salt thereof or morphic form as described herein is administered to the subject at least once daily, and wherein an effective amount of the chemotherapeutic agent is administered according to its prescribed label. In some embodiments, Compound I or a pharmaceutically acceptable salt thereof or morphic form as described herein is administered to the subject at least twice daily, and wherein an effective amount of the chemotherapeutic agent is administered according to its prescribed label. In certain embodiments, the cancer has progressed following a prior regimen comprising a CDK4/6 inhibitor. In certain embodiments, the cancer that has progressed following a prior regimen comprising a CDK4/6 inhibitor is CDK4/6 inhibitor-resistant. In some embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In some embodiments, the CDK 4/6 inhibitor-resistant cancer is lung cancer. In some embodiments, the CDK 4/6 inhibitor-resistant lung cancer is small cell lung cancer (SCLC).

### III. Pharmaceutical Compositions and Dosage Forms

Compound I or its pharmaceutically acceptable salt, or isotopic analog thereof or morphic form as described herein can be administered in an effective amount according to the methods described herein to a host to treat any of the disorders described herein using any suitable approach which achieves the desired therapeutic result. The amount and timing of Compound I administration will, of course, be dependent on the host being treated, the instructions of the supervising medical specialist, on the time course of the exposure, on the manner of administration, on the pharmacokinetic properties, and on the judgment of the prescribing physician. Thus, because of host-to-host variability, the dosages given below are a guideline and the physician can titrate doses of the compound to achieve the treatment that the physician considers appropriate for the host. In considering the degree of treatment desired, the physician can balance a variety of factors such as age and weight of the host, presence of preexisting disease, as well as presence of other diseases.

In certain embodiments the pharmaceutical composition is manufactured from a morphic form described herein. For example, in certain embodiments the pharmaceutical composition is prepared from a morphic form described herein and in the final pharmaceutical composition the compound is no longer the same morphic form or is amorphous. In certain embodiments the use of a morphic form described herein to manufacturing a pharmaceutical composition of Compound I or a pharmaceutical composition thereof provides improved purity, yield, manufacturing controls, reproducibility and/or scalability.

The pharmaceutical composition may be formulated as any pharmaceutically useful form, e.g., a liquid oral dosage form, a solid oral dosage form, a semisolid oral dosage form, as an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, intravenous solution, or an ophthalmic solution. Some dosage forms, such as tablets and capsules, are subdivided into suitably sized unit doses containing appropriate quantities of the active components, e.g., an effective amount to achieve the desired purpose.

Compounds disclosed herein or used as described herein may be administered orally, topically, parenterally, by inhalation or spray, sublingually, via implant, including ocular implant, transdermally, via buccal administration, rectally, as an ophthalmic solution, injection, including ocular injection, intravenous, intramuscular, inhalation, intra-aortal, intracranial, subdermal, intraperitoneal, subcutaneous, transnasal, sublingual, or rectal or by other means, in dosage unit formulations containing conventional pharmaceutically acceptable carriers. For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

In certain embodiments, the pharmaceutical composition comprising Compound I or a pharmaceutically acceptable salt thereof or morphic form as described herein is administered orally. For example, in certain embodiments a pharmaceutical composition comprising Compound I or a pharmaceutically acceptable salt thereof or morphic form as described herein and polyethylene glycol and/or hydroxypropyl methylcellulose.

The therapeutically effective dosage of Compound I or a pharmaceutically acceptable salt thereof or morphic form as described herein can be determined by the health care practitioner depending on the condition, size and age of the patient as well as the route of delivery. In certain non-limited embodiments, a dosage from about 0.1 to about 200 mg/kg has therapeutic efficacy, with all weights being calculated based upon the weight of the Compound I, including the cases where a salt is employed. In certain embodiments, the dosage is at about or greater than 0.1, 0.5, 1, 5, 10, 25, 50, 75, 100, 125, 150, 175, or 200 mg/kg. In some embodiments, the dosage may be the amount of compound needed to provide a serum concentration of the Compound I of up to about 10 nM, 50 nM, 100 nM, 200 nM, 300 nM, 400 nM, 500 nM, 600 nM, 700 nM, 800 nM, 900 nM, 1 μM, 5 μM, 10 μM, 20 μM, 30 μM, or 40 μM.

In certain embodiments, the pharmaceutical composition is in a dosage form that contains from about 0.1 mg to about 2000 mg, from about 10 mg to about 1000 mg, from about 100 mg to about 800 mg, or from about 200 mg to about 600 mg of the Compound I or a pharmaceutically acceptable salt thereof and optionally from about 0.1 mg to about 2000 mg, from about 10 mg to about 1000 mg, from about 100 mg to about 800 mg, or from about 200 mg to about 600 mg of an additional active agent in a unit dosage form. Examples of dosage forms with at least about 5, about 10, about 15, about 20, about 25, about 50, about 75, about 100, about 150, about 175, about 200, about 250, about 300, about 350, about 400, about 450, about 500, about 500, about 600, about 650, about 700, about 750 mg, or about 800 mg of Compound I, or its salt or morphic form as described herein. In certain embodiments, the pharmaceutical composition is in a dosage form that contains about 100 mg of Compound I, or a pharmaceutically acceptable salt thereof or morphic form as described herein. In certain embodiments, the pharmaceutical composition is in a dosage form that contains about 200 mg of Compound I, or a pharmaceutically acceptable salt thereof or morphic form as described herein. In certain embodiments, the pharmaceutical composition is in a dosage form that contains about 300 mg of Compound I, or a pharmaceutically acceptable salt thereof or morphic form as described herein. In certain embodiments, the pharmaceutical composition is in a dosage form that contains about 400 mg of Compound I, or a pharmaceutically acceptable salt thereof or morphic form as described herein. In certain embodiments, the pharmaceutical composition is in a dosage form that contains about 500 mg of Compound I, or a pharmaceutically acceptable salt thereof or morphic form as described herein. In certain embodiments, the pharmaceutical composition is in a dosage form that contains about 600 mg of Compound I, or a pharmaceutically acceptable salt thereof or morphic form as described herein. In certain embodiments, the pharmaceutical composition is in a dosage form that contains about 700 mg of Compound I, or a pharmaceutically acceptable salt thereof or morphic form as described herein. In certain embodiments, the pharmaceutical composition is in a dosage form that contains about 800 mg of Compound I, or a pharmaceutically acceptable salt thereof or morphic form as described herein. The pharmaceutical composition may also include a molar ratio of the Compound I or a pharmaceutically acceptable salt thereof and an additional active agent, in a ratio that achieves the desired results.

In some embodiments, compounds disclosed herein or a pharmaceutically acceptable salt thereof or morphic form as described herein or used as described are administered once a day (QD), twice a day (BID), or three times a day (TID). In some embodiments, Compound I or a pharmaceutically acceptable salt thereof or morphic form as described herein is administered once a day (QD). In some embodiments, Compound I or a pharmaceutically acceptable salt thereof or morphic form as described herein is administered twice a day (BID). In some embodiments, Compound I or a pharmaceutically acceptable salt thereof or morphic form as described herein is administered three times a day (TID). In some embodiments, Compound I or a pharmaceutically acceptable salt thereof or morphic form as described herein is administered at least once a day for at least 21 days, at least 24 days, at least 28 days, at least 35 days, at least 45 days, at least 60 days, at least 75 days, at least 90 days, at least 120 days, at least 180 days, or longer, indefinitely, or until the healthcare provider decides that the drug is no longer necessary.

In accordance with the presently disclosed methods, an oral administration can be in any desired form such as a solid, gel or liquid, including a solution, suspension, or emulsion. In some embodiments, the compounds or salts are administered by inhalation, intravenously, or intramuscularly as a liposomal suspension. When administered through inhalation Compound I or salt or morphic form as described herein may be in the form of a plurality of solid particles or droplets having any desired particle size, and for example, from about 0.01, 0.1 or 0.5 to about 5, 10, 20 or more microns, and optionally from about 1 to about 2 microns. Compounds as disclosed in the present invention have demonstrated good pharmacokinetic and pharmacodynamics properties, for instance when administered by the oral or intravenous routes.

The pharmaceutical formulations can comprise Compound I or a pharmaceutically acceptable salt thereof or morphic form as described herein, in any pharmaceutically acceptable carrier. If a solution is desired, water may sometimes be the carrier of choice for water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and for illustration by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound.

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

Thus, the compositions of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, Compound I or a pharmaceutically acceptable salt thereof or morphic form as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or can be apparent, to those skilled in this art; for example, see Remington's Pharmaceutical Sciences, referenced above.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as an continuous infusion system. A formulation provided by the disclosure can be administered using a syringe, injector, pump, or any other device recognized in the art for parenteral administration.

In addition to Compound I or a pharmaceutically acceptable salt thereof or morphic form as described herein, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulations is placed in a vial designed for multi-dose use. The pharmaceutical formulations described herein can be lyophilized using techniques well known in the art.

For oral administration, a pharmaceutical composition can take the form of a solution suspension, tablet, pill, capsule, powder, and the like. Tablets containing various excipients such as sodium citrate, calcium carbonate and calcium phosphate may be employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate, and talc are often very useful for tableting purposes. Solid compositions of a similar type may be employed as fillers in soft and hard-filled gelatin capsules. Materials in this connection also include lactose or milk sugar as well as high molecular weight polyethylene glycols. When aqueous suspensions and/or elixirs are desired for oral administration, the compounds of the presently disclosed host matter can be combined with various sweetening agents, flavoring agents, coloring agents, emulsifying agents and/or suspending agents, as well as such diluents as water, ethanol, propylene glycol, glycerin and various like combinations thereof.

In yet another embodiment of the host matter described herein, there are provided injectable, stable, sterile formulations comprising an active compound as described herein, or a salt thereof, in a unit dosage form in a sealed container. The compound or salt is provided in the form of a lyophilizate, which is capable of being reconstituted with a suitable pharmaceutically acceptable carrier to form liquid formulation suitable for injection thereof into a host. When the compound or salt is substantially water-insoluble, a sufficient amount of emulsifying agent, which is physiologically acceptable, can be employed in sufficient quantity to emulsify the compound or salt in an aqueous carrier. Particularly useful emulsifying agents include phosphatidyl cholines and lecithin.

Additional embodiments include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulations can be placed in a small chamber and nebulized. Nebulization can be accomplished by compressed air or by ultrasonic energy to form a plurality of liquid droplets or solid particles comprising the compounds or salts. The liquid droplets or solid particles may for example have a particle size in the range of about 0.5 to about 10 microns, and optionally from about 0.5 to about 5 microns. In certain embodiments, the solid particles provide for controlled release through the use of a degradable polymer. The solid particles can be obtained by processing the solid compound or a salt thereof, in any appropriate manner known in the art, such as by micronization. Optionally, the size of the solid particles or droplets can be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

Pharmaceutical formulations also are provided which provide a controlled release of a compound described herein, including through the use of a degradable polymer, as known in the art.

When the pharmaceutical formulations suitable for administration as an aerosol is in the form of a liquid, the formulations can comprise a water-soluble active compound in a carrier that comprises water. A surfactant can be present, which lowers the surface tension of the formulations sufficiently to result in the formation of droplets within the desired size range when hosted to nebulization.

The term “pharmaceutically acceptable salts” as used herein refers to those salts which are, within the scope of sound medical judgment, suitable for use in contact with hosts (e.g., human hosts) without undue toxicity, irritation, allergic response, and the like, commensurate with a reasonable benefit/risk ratio, and effective for their intended use, as well as the zwitterionic forms, where possible, of the compounds of the presently disclosed host matter.

Thus, the term “salts” refers to the relatively non-toxic, inorganic and organic acid addition salts of the presently disclosed compounds. These salts can be prepared during the final isolation and purification of the compounds or by separately reacting the purified compound in its free base form with a suitable organic or inorganic acid and isolating the salt thus formed. Basic compounds are capable of forming a wide variety of different salts with various inorganic and organic acids. Acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents.

Salts can be prepared from inorganic acids include hydrochloric, sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Formulations suitable for rectal administration are typically presented as unit dose suppositories. These may be prepared by admixing the active disclosed compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6): 318 (1986)) and typically take the form of an optionally buffered aqueous solution of the active compound. In certain embodiments, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

### IV. Combination Therapy

In certain aspects, Compound I or a pharmaceutically acceptable salt thereof or morphic form as described herein is used in an effective amount alone or in combination with another bioactive agent (therapeutic agent) to treat a subject such as a human with cyclin E overexpressing or amplified disorder described herein or small cell lung cancer is provided.

In other aspects, a pharmaceutical composition comprising a morphic form of Compound I or a pharmaceutically acceptable salt thereof is used in an effective amount alone or in combination with another bioactive agent (therapeutic agent) to treat a subject such as a human with a CDK2 mediated cancer or small cell lung cancer described herein is provided.

In yet other aspects, a pharmaceutical composition comprising Compound I or a pharmaceutically acceptable salt thereof or morphic form as described herein is used in an effective amount alone or in combination with another bioactive agent (therapeutic agent) to treat a subject such as a human with a CDK2 mediated cancer or small cell lung cancer described herein is provided, wherein the pharmaceutical composition is prepared from a morphic form of Compound I or a pharmaceutically acceptable salt thereof or morphic form as described herein (for example by spray drying or dissolving the morphic form and then mixing it with one or more pharmaceutically acceptable excipients).

The term “bioactive agent” or “therapeutic agent” or “anti-cancer therapy” is used to describe an agent, other than Compound I, which can be used in combination or alternation with Compound I to achieve a desired result of therapy. In certain embodiments, Compound I and the bioactive agent are administered in a manner that they are active in vivo during overlapping time periods, for example, have time-period overlapping Cₘₐₓ, Tₘₐₓ, AUC, or other pharmacokinetic parameter. In another embodiment, Compound I and the bioactive agent are administered to a subject in need thereof that do not have overlapping pharmacokinetic parameter, however, one has a therapeutic impact on the therapeutic efficacy of the other.

Despite the successful treatment of many patients with CDK4/6 inhibitors, a major problem in the treatment of cancer is that a sizable population of patients are inherently unresponsive to the treatment, and a significant proportion of the remaining patients eventually develop resistance to CDK4/6 inhibition (Xu et al. Cureus. 9: e1408(2017); Condorelli et al. Annals of Oncology. 29:640-5(2018)). For example, MYC-type tumors, such as triple negative breast cancer (TNBC) and small cell lung cancer (SCLC), exhibit loss of retinoblastoma (Rb) protein expression. Certain cancers, despite being Rb-positive, are intrinsically resistant to the effects of selective CDK4/6 inhibitors. Furthermore, certain cancers that have an intact Rb-pathway may otherwise be intrinsically resistant to selective CDK4/6 inhibitor due to the presence of other genetic or phenotypical abnormalities. For example, it is estimated that 40% of uterine, 20% of ovarian, 15% of bladder, 20% or prostate, and 15% of breast cancers may be intrinsically resistant to selective CDK4/6 inhibition due to the upregulation of Cyclin E, despite intact Rb. See, e.g., Knudsen et al., The Strange Case of CDK4/6 Inhibitors: Mechanisms, Resistance, and Combination Strategies. Trends Cancer. 2017 January; 3(1): 39-55. Other cancers, for example ER+ breast cancers, acquire resistance to selective CDK4/6 inhibitors during the course of selective CDK4/6 inhibitor therapy, for example by upregulation of cyclin E, which allows G1 to S cell cycle progression through CDK2. In certain embodiments, Compound I effectively inhibits cell-cycle progression in cancer cells that are intrinsically resistant to, susceptible to acquiring resistance to, or have become resistant to selective CDK4/6 inhibitors. In certain embodiments, Compound I effectively inhibits cell-cycle progression in cancer cells of a subject having a cancer that has progressed following a prior regimen comprising a CDK4/6 inhibitor.

In one aspect, disclosed herein is a method for treating a subject with a CDK4/6-resistant cancer comprising administering to the subject an effective amount of Compound I, or a pharmaceutically acceptable salt thereof or morphic form as described herein; and, administering to the subject an effective amount of a CDK4/6 inhibitor. In some embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In some embodiments, the CDK4/6 inhibitor-resistant cancer is retinoblastoma (Rb) protein-positive (Rb+). In some embodiments, the CDK4/6 inhibitor-resistant cancer is retinoblastoma (Rb) protein-null (Rb−). In some embodiments, the CDK4/6-inhibitor resistant cancer is selected from breast cancer, lung cancer, uterine cancer, endometrial cancer, ovarian cancer, prostate cancer, bladder cancer, testicular cancer, glioblastoma, head and/or neck cancer, or prostate cancer. In some embodiments, the CDK4/6-inhibitor resistant cancer is breast cancer. In some embodiments, the CDK4/6-inhibitor resistant breast cancer is estrogen receptor-positive (ER+) breast cancer. In some embodiments, the CDK4/6-inhibitor resistant breast cancer is hormone receptor-positive (HR+) breast cancer. In certain embodiments, the breast cancer is human epidermal growth factor receptor 2 negative (HER2−). In certain embodiments, the breast cancer is ER+/HER2− breast cancer. In certain embodiments, the breast cancer is HR+/HER2-breast cancer. In certain embodiments, the CDK4/6-inhibitor resistant cancer is ovarian cancer. In certain embodiments, the ovarian cancer has an amplification of CCNE1. In certain embodiments, the method further comprises administering an effective amount of an estrogen inhibitor. In certain embodiments, the estrogen inhibitor is a selective estrogen receptor degrader (SERD). In certain embodiments, the SERD is selected from fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, or ZN-c5. In some embodiments, the SERD comprises fulvestrant. In some embodiments, the SERD comprises elacestrant (RAD1901).

In certain embodiments, the methods specifically described above further comprises the administration of an effective amount of an additional anti-cancer therapy. In some embodiments, the anti-cancer therapy is selected a chemotherapeutic agent, radiation, surgery, immune checkpoint inhibitor, estrogen inhibitor, androgen inhibitor, PARP inhibitor, or a combination thereof. In some embodiments, the anti-cancer therapy comprises a chemotherapeutic agent. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof. In some embodiments, the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof. In some embodiments, Compound I and the CDK4/6 inhibitor are administered within 24 hours or less to the administration of the chemotherapeutic agent. In some embodiments, Compound I and the CDK4/6 inhibitor are administered within 6 hours or less to the administration of the chemotherapeutic agent. In some embodiments, Compound I and the CDK4/6 inhibitor are administered within 3 hours or less of the administration of the chemotherapeutic agent. In some embodiments, Compound I or a pharmaceutically acceptable salt thereof or morphic form as described herein is administered to the subject at least once daily, and wherein an effective amount of the CDK4/6 inhibitor and chemotherapeutic agent are administered according to their prescribed labels. In some embodiments, Compound I or a pharmaceutically acceptable salt thereof or morphic form as described herein is administered to the subject at least twice daily, and wherein an effective amount of the CDK4/6 inhibitor and chemotherapeutic agent are administered according to their prescribed labels.

In another aspect, disclosed herein is a method for treating a subject with a CDK4/6 inhibitor-resistant estrogen receptor-positive (ER+) breast cancer comprising administering to the subject an effective amount of Compound I, or a pharmaceutically acceptable salt thereof or morphic form as described herein; and, administering to the subject an effective amount of a CDK4/6 inhibitor. In some embodiments, the CDK4/6 inhibitor-resistant ER+ breast cancer is tyrosine kinase-type cell surface receptor HER2-negative. In certain embodiments, the method further comprises administering an effective amount of an estrogen inhibitor. In certain embodiments, the estrogen inhibitor is a selective estrogen receptor degrader (SERD). In some embodiments, the SERD comprises fulvestrant. In some embodiments, the SERD comprises elacestrant (RAD1901). In some embodiments, the method further comprises administering an effective amount of an antibody drug conjugate (ADC). In some embodiments, the method further comprises administering an effective amount of an ADC selected from ado-trastuzumab emtansine (KADCYLA®), trastuzumab deruxtecan (ENHERTU®), or sacituzumab govetican (TRODELVY®).

In some embodiments, disclosed herein are methods for treating a human with a CDK4/6 inhibitor-resistant and optionally endocrine therapy-resistant cancer comprising administering an effective amount of Compound I, or pharmaceutically acceptable salt thereof or morphic form as described herein, and administering an effective amount of an estrogen inhibitor. In some embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In some embodiments, the estrogen inhibitor is a selective estrogen receptor degrader (SERD). In some embodiments, the SERD comprises fulvestrant. In some embodiments, the SERD comprises elacestrant (RAD1901). Additional non-limiting examples of anti-estrogen compounds include: SERMS such as anordrin, arzoxifene, bazedoxifene, broparestriol, clomiphene citrate, cyclofenil, droloxifene, endoxifen, idoxifene, lasofoxifene, ormeloxifene, pipendoxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, delmadinone acetate, dydrogesterone, medroxyprogesterone acetate, megestrol acetate, nomegestrol acetate, norethisterone acetate, progesterone, and spironolactone. Additional non-limiting examples of anti-estrogen compounds include: SERDS such as fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, and ZN-c5. In some embodiments, the SERD is elacestrant (RAD1901). In some embodiments, the SERD is fulvestrant. In some embodiments, the human previously received at least one prior line of endocrine therapy. In some embodiments, the human previously received at least one prior line of CDK4/6 inhibitor therapy. In some embodiments, the human previously received at least one prior line of chemotherapy. In some embodiments, the human previously received at least two prior lines of chemotherapy. In some embodiments, the cancer has progressed following a prior regimen comprising a CDK4/6 inhibitor. In certain embodiments, the method further comprises administering an effective amount of an additional anti-cancer therapy. In some embodiments, the anti-cancer therapy is selected from a chemotherapeutic agent, radiation, surgery, immune checkpoint inhibitor, CDK4/6 inhibitor, estrogen inhibitor, androgen inhibitor, PARP inhibitor, or a combination thereof. In some embodiments, the method further comprises administering an effective amount of a CDK4/6 inhibitor. In some embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the cancer is selected from breast cancer, ovarian cancer, endometrial cancer, prostate cancer, or uterine cancer. In some embodiments, the cancer is breast cancer. In some embodiments, the breast cancer is ER+ breast cancer. In some embodiments, the breast cancer is HR+ breast cancer. In some embodiments, the breast cancer is PR+ breast cancer. In some embodiments, the breast cancer is HER2− breast cancer. In some embodiments, the breast cancer is ER+ HER2− breast cancer. In some embodiments, the breast cancer is ER+ PR+ HER2− breast cancer. In some embodiments, the breast cancer is HR+ HER2− breast cancer. In some embodiments, the CDK4/6 inhibitor-resistant and/or estrogen inhibitor-resistant breast cancer is luminal A breast cancer.

In some embodiments, disclosed herein are methods for treating a human having advanced unresectable or metastatic breast cancer comprising: administering to the subject an effective amount of Compound I, or a pharmaceutically acceptable salt thereof or morphic form as described herein; administering to the subject an effective amount of an estrogen inhibitor; and optionally, administering to the subject an effective amount a CDK4/6 inhibitor. In some embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In some embodiments, the estrogen inhibitor is a selective estrogen receptor degrader (SERD). In some embodiments, the SERD is selected from fulvestrant, rintodestrant (G1T48), brilanestrant (GDC0810), elacestrant (RAD1901), etacstil (GW5638), GW7604, AZD9496, GDC-0927, GDC9545 (RG6171), LSZ102, or SAR439859. In some embodiments, the SERD comprises fulvestrant. In some embodiments, the SERD comprises elacestrant (RAD1901). In some embodiments, the estrogen inhibitor is a selective estrogen receptor modulator (SERM). In some embodiments, the SERM is selected from anordrin, arzoxifene, bazedoxifene, broparestriol, clomiphene citrate, cyclofenil, droloxifene, endoxifen, idoxifene, lasofoxifene, ormeloxifene, pipendoxifene, raloxifene, tamoxifen, toremifene, aminoglutethimide, testolactone, anastrozole, exemestane, fadrozole, formestane, letrozole, leuprorelin, cetrorelix, allylestrenol, chloromadinone acetate, delmadinone acetate, dydrogesterone, medroxyprogesterone acetate, megestrol acetate, nomegestrol acetate, norethisterone acetate, progesterone, or spironolactone. In some embodiments, the SERD comprises fulvestrant. In some embodiments, the SERD comprises elacestrant (RAD1901). Selective CDK4/6 inhibitors for use in combination with Compound I include, but are not limited to palbociclib, abemaciclib, ribociclib, trilaciclib, SHR6390 (dalpiciclib), and lerociclib. In some embodiments, the CDK 4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor for use in combination with Compound I is palbociclib. In some embodiments, the CDK4/6 inhibitor for use in combination with Compound I is ribociclib. In some embodiments, the CDK4/6 inhibitor for use in combination with Compound I is abemaciclib. In certain embodiments, the advanced unresectable and/or metastatic ER+/HER2− breast cancer has progressed following treatment with a CDK4/6 inhibitor. In certain embodiments, the previously administered CDK4/6 inhibitor is selected from palbociclib, abemaciclib, ribociclib, trilaciclib, SHR6390 (dalpiciclib), or lerociclib. In some embodiments, the previously administered CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.

In another aspect, disclosed herein are methods for treating a subject with breast cancer, comprising administering to the subject an effective amount of Compound I, or a pharmaceutically acceptable salt thereof or morphic form as described herein, and administering to the subject an effective amount of an estrogen inhibitor. In some embodiments, the breast cancer is ER+ breast cancer. In some embodiments, the breast cancer is HR+ breast cancer. In some embodiments, the breast cancer is PR+ breast cancer. In some embodiments, the breast cancer is HER2− breast cancer. In some embodiments, the breast cancer is ER+ HER2− breast cancer. In some embodiments, the breast cancer is ER+ PR+ HER2− breast cancer. In some embodiments, the breast cancer is HR+ HER2− breast cancer. In certain embodiments, the method is administered as a first-line (1L) therapy. In some embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In some embodiments, the estrogen inhibitor is a selective estrogen receptor degrader (SERD). In certain embodiments, the SERD is selected from fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, or ZN-c5. In some embodiments, the SERD comprises fulvestrant. In some embodiments, the SERD comprises elacestrant (RAD1901). In some embodiments, the estrogen inhibitor is a selective estrogen receptor modulator (SERM). In some embodiments, the SERM is selected from anordrin, arzoxifene, bazedoxifene, broparestriol, clomiphene citrate, cyclofenil, droloxifene, endoxifen, idoxifene, lasofoxifene, ormeloxifene, pipendoxifene, raloxifene, tamoxifen, toremifene, aminoglutethimide, testolactone, anastrozole, exemestane, fadrozole, formestane, letrozole, leuprorelin, cetrorelix, allylestrenol, chloromadinone acetate, delmadinone acetate, dydrogesterone, medroxyprogesterone acetate, megestrol acetate, nomegestrol acetate, norethisterone acetate, progesterone, or spironolactone. In some embodiments, the SERM is selected from anastrazole, exemestane, or letrozole. In certain embodiments, the method further comprises administering an effective amount of a CDK4/6 inhibitor. In some embodiments, the CDK4/6 inhibitor is selected from palbociclib, ribociclib, abemaciclib, trilaciclib, lerociclib, or SHR6390 (dalpiciclib). In some embodiments, the CDK 4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In some embodiments, the CDK4/6 inhibitor is palbocicilib and abemaciclib. In certain embodiments, the method prolongs the time to acquired resistance to the estrogen inhibitor in comparison to a method lacking Compound I. In certain embodiments, the method prolongs the time to acquired resistance to the CDK4/6 inhibitor in comparison to a method lacking Compound I. In certain embodiments, the subject previously received at least one prior line of endocrine therapy. In certain embodiments, the breast cancer has progressed following a prior standard of care regimen. In some embodiments, the CDK4/6 inhibitor-resistant and/or estrogen inhibitor-resistant breast cancer is luminal A breast cancer.

In another aspect, disclosed herein are methods for treating a subject with estrogen receptor positive (ER+) advanced breast cancer, comprising administering to the subject an effective amount of Compound I, or a pharmaceutically acceptable salt thereof or morphic form as described herein, and administering to the subject an effective amount of fulvestrant. In certain embodiments, the HR+ advanced breast cancer is human epidermal growth factor 2 negative (HER2−). In certain embodiments, the method is administered as a first-line (1L) therapy. In certain embodiments, the method prolongs the time to acquired resistance to fulvestrant in comparison to a method lacking Compound I. In certain embodiments, the subject previously received at least one prior line of endocrine therapy. In certain embodiments, the ER+ or ER+/HER2− breast cancer has progressed following a prior standard of care regimen. In certain embodiments, the method further comprises administering an effective amount of a CDK4/6 inhibitor. In some embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In some embodiments, the CDK4/6 inhibitor is palbocicilib and abemaciclib.

In another aspect, disclosed herein are methods for treating a subject with hormone receptor positive (HR+) advanced breast cancer, comprising administering to the subject an effective amount of Compound I, or a pharmaceutically acceptable salt thereof or morphic form as described herein, and administering to the subject an effective amount of fulvestrant. In certain embodiments, the HR+ advanced breast cancer is human epidermal growth factor 2 negative (HER2−). In certain embodiments, the method is administered as a first-line (IL) therapy. In certain embodiments, the method prolongs the time to acquired resistance to fulvestrant in comparison to a method lacking Compound I. In certain embodiments, the subject previously received at least one prior line of endocrine therapy. In certain embodiments, the HR+ or HR+/HER2− breast cancer has progressed following a prior standard of care regimen. In certain embodiments, the method further comprises administering an effective amount of a CDK4/6 inhibitor. In some embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In some embodiments, the CDK4/6 inhibitor is palbocicilib and abemaciclib.

In another aspect, disclosed fully herein are methods for treating a subject with an unresectable cancer by administering to the subject a morphic form of Compound I as described herein. In another aspect, disclosed fully herein are methods for treating a subject with an advanced cancer by administering to the subject a morphic form of Compound I as described herein. In another aspect, disclosed herein are methods for treating a subject with a metastatic cancer by administering to the subject a morphic form of Compound I as described herein. In another aspect, disclosed herein s method for treating a subject with an advanced unresectable and/or metastatic cancer by administering to the subject a morphic form of Compound I as described herein. In certain embodiments, the advanced unresectable and/or metastatic cancer is selected from uterine cancer, uterine carcinosarcoma (UCS), uterine corpus endometrial carcinoma (UCEC), ovarian cancer, ovarian serous cystadenocarcinoma (OV), sarcoma (SARC), lung cancer, lung squamous cell carcinoma (LUSC), lung adenocarcinoma (LUAD), stomach cancer, stomach adenocarcinoma (STAD), bladder cancer, bladder urothelial carcinoma (BLCA), esophageal cancer, esophageal carcinoma (ESCA), adrenocortical carcinoma, breast cancer, breast invasive carcinoma (BRCA), pancreatic cancer, pancreatic adenocarcinoma (PAAD), fallopian tube cancer, primary peritoneal cancer, liver cancer, liver hepatocellular carcinoma (LIHC), cervical cancer, cervical squamous cell carcinoma (CESC), endocervical adenocarcinoma, mesothelioma (MESO), head and neck squamous cell carcinoma (HSNC), colon cancer, colon adenocarcinoma (COAD), skin cancer, melanoma, skin cutaneous melanoma (SKCM), glioblastoma multiforme (GBM), kidney cancer, or kidney chromophobe (KICH). In some embodiments, the cyclin E amplified or overexpressed cancer is retinoblastoma (Rb) protein positive. In some embodiments, the cyclin E amplified or overexpressed cancer is CDK4/6 inhibitor-resistant. In certain embodiments, the cancer is advanced and/or metastatic cancer. In certain embodiments, the cancer is advanced unresectable cancer. In certain embodiments, the cancer is platinum-refractory and/or platinum-resistant. In certain embodiments, the cancer has progressed following a prior standard of care regimen. In certain embodiments, the cancer has progressed following a prior standard systemic therapy. In certain embodiments, the cancer has progressed following a prior systemic anti-cancer therapy. In certain embodiments, the cancer has progressed following a prior regimen comprising a platinum analog. In certain embodiments, the cancer has progressed following a prior regimen comprising a CDK4/6 inhibitor. In certain embodiments, the method further comprises administering an effective amount of a CDK4/6 inhibitor. In certain embodiments, the CDK4/6 inhibitor is selected from palbociclib, ribociclib, abemaciclib, trilaciclib, lerociclib, or SHR6390 (dalpiciclib). In some embodiments, the CDK 4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In certain embodiments, the method further comprises administering an effective amount of an additional anti-cancer therapy. In some embodiments, the anti-cancer therapy is selected from a chemotherapeutic agent, radiation, surgery, immune checkpoint inhibitor, estrogen inhibitor, androgen inhibitor, PARP inhibitor, or a combination thereof. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof. In certain embodiments, the anti-cancer therapy is an estrogen inhibitor. In certain embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In certain embodiments, the estrogen inhibitor is a selective estrogen receptor degrader (SERD). In some embodiments, the SERD comprises fulvestrant. In some embodiments, the SERD comprises elacestrant (RAD1901).

In some embodiments, disclosed herein are methods for treating a human with an advanced unresectable and/or metastatic estrogen receptor-positive (ER+) epidermal growth factor receptor 2 negative (HER2−) breast cancer comprising administering an effective amount of Compound I, or pharmaceutically acceptable salt thereof or morphic form as described herein. In some embodiments, the human previously received at least one prior line of endocrine therapy. In some embodiments, the human previously received at least one prior line of CDK4/6 inhibitor therapy. In some embodiments, the human previously received at least one prior line of chemotherapy. In some embodiments, the human previously received at least two prior lines of chemotherapy. In some embodiments, the advanced unresectable or metastatic ER+/HER2− breast cancer has progressed following a prior regimen comprising a CDK4/6 inhibitor. In certain embodiments, the method further comprises administering an effective amount of a CDK4/6 inhibitor. In certain embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In certain embodiments, the method further comprises administering an effective amount of an additional anti-cancer therapy. In some embodiments, the anti-cancer therapy is selected from a chemotherapeutic agent, radiation, surgery, immune checkpoint inhibitor, estrogen inhibitor, androgen inhibitor, PARP inhibitor, or a combination thereof. In some embodiments, the chemotherapeutic agent is selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof. In certain embodiments, the anti-cancer therapy is an estrogen inhibitor. In certain embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof. In certain embodiments, the estrogen inhibitor is a selective estrogen receptor degrader (SERD). In some embodiments, the SERD comprises fulvestrant. In some embodiments, the SERD comprises elacestrant (RAD1901). In some embodiments, disclosed herein is a method for treating a human having advanced unresectable or metastatic ER+/HER2− breast cancer comprising: administering to the subject an effective amount of Compound I, or a pharmaceutically acceptable salt thereof or morphic form described herein; administering to the subject an effective amount of a CDK4/6 inhibitor; and, administering to the subject an effective amount of a selective estrogen receptor degrader (SERD). In some embodiments, the SERD comprises fulvestrant. In some embodiments, the SERD comprises elacestrant (RAD1901). In certain embodiments, the advanced unresectable and/or metastatic ER+/HER2− breast cancer has progressed following treatment with a CDK4/6 inhibitor. In certain embodiments, the previously administered CDK4/6 inhibitor is selected from palbociclib, abemaciclib, ribociclib, trilaciclib, SHR6390 (dalpiciclib), or lerociclib. Selective CDK4/6 inhibitors for use in combination with Compound I include, but are not limited to palbociclib, abemaciclib, ribociclib, trilaciclib, SHR6390 (dalpiciclib), and lerociclib. In alternative embodiments, the CDK4/6 inhibitor for use in combination with Compound I is selected from BPI-16350, narazaciclib (ON-123300), FLX-925 (AMG-925), UCT-03-008, GLR2007, birociclib (XZP-3287), LY5219, PF-07220060, or ON-123300

In certain embodiments, the methods as described herein further comprise administering an effective amount of an estrogen inhibitor. In some embodiments, the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.

In certain embodiments, the methods as described herein further comprises administering an effective amount of a chemotherapeutic agent. In some embodiments, the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof. In some embodiments, the CDK4/6 inhibitor-resistant ER+ breast cancer is luminal A breast cancer. In some embodiments, Compound I or a pharmaceutically acceptable salt thereof is administered to the subject at least once daily, and wherein an effective amount of the chemotherapeutic agent is administered according to its prescribed label. In some embodiments, Compound I or a pharmaceutically acceptable salt thereof is administered to the subject at least twice daily, and wherein an effective amount of the chemotherapeutic agent is administered according to its prescribed label.

In some embodiments, disclosed herein are methods for treating a human with a CDK4/6 inhibitor-resistant cancer comprising administering Compound I, a pharmaceutically acceptable salt thereof or morphic form described herein, in combination with a CDK4/6 inhibitor. In some embodiments, the CDK4/6 inhibitor is selected from palbociclib, ribociclib, abemaciclib, trilaciclib, lerociclib, or SHR6390 (dalpiciclib). In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the CDK4/6 inhibitor is palbociclib. In some embodiments, the CDK4/6 inhibitor is ribociclib. In some embodiments, the CDK4/6 inhibitor is abemaciclib. In some embodiments, the CDK4/6 inhibitor-resistant cancer is lung cancer. In some embodiments, the CDK4/6 inhibitor-resistant lung cancer is small cell lung cancer (SCLC).

In another aspect, disclosed herein are methods for treating a subject with a CDK4/6 inhibitor-resistant small cell lung cancer (SCLC) comprising administering to the subject an effective amount of Compound I, or a pharmaceutically acceptable salt thereof or morphic form as described herein; and administering to the subject an effective amount of a chemotherapeutic agent, wherein Compound I is administered to the subject within 24 hours or less to the administration of the chemotherapeutic agent. In some embodiments, the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof. In some embodiments, the chemotherapeutic agent is doxorubicin. In some embodiments, the chemotherapeutic agent is camptothecin. In some embodiments, the chemotherapeutic agent is cisplatin. In some embodiments, the chemotherapeutic agent is carboplatin. In some embodiments, the chemotherapeutic agent is etoposide. In some embodiments, Compound I is administered to the subject within 6 hours or less to the administration of the chemotherapeutic agent. In some embodiments, Compound I is administered to the subject within 3 hours or less to the administration of the chemotherapeutic agent. In some embodiments, Compound I or a pharmaceutically acceptable salt thereof is administered to the subject at least once daily, and wherein an effective amount of the chemotherapeutic agent is administered according to its prescribed label. In some embodiments, Compound I or a pharmaceutically acceptable salt thereof is administered to the subject at least twice daily, and wherein an effective amount of the chemotherapeutic agent is administered according to its prescribed label.

In certain embodiments, the methods as described herein further comprise administering an effective amount of an additional anti-cancer therapy. In some embodiments, the anti-cancer therapy is selected from radiation, surgery, immune checkpoint inhibitor, estrogen inhibitor, androgen inhibitor, PARP inhibitor, or a combination thereof.

In another aspect, disclosed herein are methods for treating a subject with a CDK4/6 inhibitor-resistant small cell lung cancer (SCLC) comprising administering to the subject an effective amount of Compound I, or a pharmaceutically acceptable salt thereof or morphic form as described herein; and administering to the subject an effective amount of doxorubicin, wherein Compound I is administered to the subject within 24 hours or less prior to or concomitantly with the administration of doxorubicin. In some embodiments, Compound I is administered to the subject within 6 hours or less prior to or concomitantly with the administration of doxorubicin. In some embodiments, Compound I is administered to the subject within 3 hours or less prior to or concomitantly with the administration of doxorubicin.

In certain aspects of this embodiment, the bioactive agent is a chemotherapeutic agent.

In another aspect of this embodiment, the bioactive agent is a growth factor.

In certain aspects of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitor, small molecule, peptide, nucleotide, or other inhibitor. In certain aspects, the immune modulator is an antibody, such as a monoclonal antibody.

### Immune Checkpoint Inhibitors

In an alternative aspect, the selective CDK2 inhibitor Compound I or pharmaceutically acceptable salt thereof or morphic form as described herein is administered to the subject in combination with an effective amount of an immune checkpoint inhibitor. Immune checkpoint inhibitors for use in the methods described herein include, but are not limited to programmed cell death-1 (PD-1) inhibitors, programmed cell death ligand 1 (PD-L1) inhibitors, programmed cell death ligand 2 (PD-L2) inhibitors, cytotoxic T-lymphocyte-associated protein 4 (CTLA-4) inhibitors, lymphocyte-activation gene 3 (LAG-3) inhibitors, T-cell immunoglobulin mucin-3 (TIM-3) inhibitors, T cell immunoreceptor with Ig and ITIM domains (TIGIT) inhibitors, V-domain Ig suppressor of T-cell activation (VISTA) inhibitors, B7-H3/CD276 inhibitors, indoleamine 2,3-dioxygenase (IDO) inhibitors, killer immunoglobulin-like receptors (KIRs) inhibitors, carcinoembryonic antigen cell adhesion molecules (CEACAM) inhibitors, sialic acid-binding immunoglobulin-like lectin 15 (Siglec-15) inhibitors, CD47 inhibitors, CD39 inhibitors, B and T lymphocyte attenuator (BTLA) protein inhibitors, or combinations thereof. In some embodiments, an immune checkpoint inhibitor is administered in an effective amount in combination with a compound described herein to treat a cancer, including but not limited to, Hodgkin lymphoma, melanoma, non-small cell lung cancer, including NSCLC with EGFR or ALK genomic tumor aberrations, squamous cell carcinoma of the head and neck, small cell lung cancer, hepatocellular carcinoma, renal cell carcinoma, urothelial carcinoma, colorectal cancer, colorectal cancer, hepatocellular carcinoma, renal cell carcinoma, small-cell lung carcinoma, bladder carcinoma, B-cell lymphoma, gastric cancer, cervical cancer, liver cancer, advanced Merkel cell carcinoma, esophageal squamous cell carcinoma, or ovarian cancer.

### PD-1 Inhibitors

In certain embodiments, the immune checkpoint inhibitor is a PD-1 inhibitor that blocks the interaction of PD-1 and PD-L1 by binding to the PD-1 receptor, and in turn inhibits immune suppression. In certain embodiments, the immune checkpoint inhibitor is a PD-1 immune checkpoint inhibitor selected from nivolumab (Opdivo®), pembrolizumab (Keytruda®), pidilizumab, AMP-224 (Amplimmune), sasanlimab (PF-06801591; Pfizer), MEDI0680 (AstraZeneca), spartalizumab (PDR001; Novartis), cemiplimad (Libtayo®; REGN2810; Regeneron), retifanlimab (MGA012; MacroGenics), islelizumab (BGB-A317; BeiGene), camrelizumab (SHR-12-1; Jiangsu Hengrui Medicine Company and Incyte Corporation), CS1003 (Cstone Pharmaceuticals), dostarlimab (TSR-042; Tesaro), and the PD-L1/VISTA inhibitor CA-170 (Curis Inc.).

In certain embodiments, the immune checkpoint inhibitor is the PD-1 immune checkpoint inhibitor nivolumab (Opdivo®) administered in an effective amount with a compound described herein for the treatment of Hodgkin lymphoma, melanoma, non-small cell lung cancer, including NSCLC with EGFR or ALK genomic tumor aberrations, squamous cell carcinoma of the head and neck, small cell lung cancer, hepatocellular carcinoma, renal cell carcinoma, squamous cell carcinoma, urothelial carcinoma, colorectal cancer, colorectal cancer, hepatocellular carcinoma, or ovarian cancer. Nivolumab has been FDA approved for the use of Hodgkin lymphoma, melanoma, non-small cell lung cancer, including NSCLC with EGFR or ALK genomic tumor aberrations, squamous cell carcinoma of the head and neck, small cell lung cancer, hepatocellular carcinoma, renal cell carcinoma, squamous cell carcinoma, urothelial carcinoma, colorectal cancer, progressive classical Hodgkin lymphoma (cHL), colorectal cancer, urothelial cancer, squamous cell carcinoma of the head and neck, or ovarian cancer. In some embodiments, nivolumab is administered at 240 mg every 2 weeks or 480 mg every 4 weeks. In some embodiments, the PD-1 inhibitor is pembrolizumab (Keytruda®) administered in an effective amount. In some embodiments, pembrolizumab is administered at 200 mg every 3 weeks or 400 mg every 6 weeks. In another aspect of this embodiment, the immune checkpoint inhibitor is the PD-1 immune checkpoint inhibitor pembrolizumab (Keytruda®) administered in an effective amount for the treatment of melanoma, non-small cell lung cancer, small cell lung cancer, head and neck cancer, bladder cancer, urothelial carcinoma, renal cell carcinoma, classical Hodgkin lymphoma, gastric cancer, cervical cancer, liver cancer, primary mediastinal B-cell lymphoma, advanced Merkel cell carcinoma, esophageal squamous cell carcinoma, or urothelial cancer. In an additional aspect of this embodiment, the immune checkpoint inhibitor is the PD-1 immune checkpoint inhibitor pidilizumab (Medivation) administered in an effective amount for refractory diffuse large B-cell lymphoma (DLBCL) or metastatic melanoma. In an additional aspect of this embodiment, the immune checkpoint inhibitor is the PD-1 immune checkpoint inhibitor cemiplimab (Libtayo/Regeneron) administered in an effective amount for cutaneous squamous cell carcinoma.

### PD-LI Inhibitors

In certain embodiments, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, atezolizumab (Tecentriq®, Genentech), durvalumab (Imfinzi®, AstraZeneca); avelumab (Bavencio®; Merck), envafolimab (KN035; Alphamab), BMS-936559 (Bristol-Myers Squibb), lodapolimab (LY3300054; Eli Lilly), cosibelimab (CK-301; Checkpoint Therapeutics), sugemalimab (CS-1001; Cstone Pharmaceuticals), adebrelimab (SHR-1316; Jiangsu HengRui Medicine), CBT-502 (CBT Pharma), and BGB-A333 (BeiGene). In certain embodiments, the PD-L1 inhibitor is atezolizumab. In certain embodiments, the PD-L1 inhibitor is durvalumab. In certain embodiments, the PD-L1 inhibitor is avelumab. In certain embodiments, the PD-L1 inhibitor blocks the interaction between PD-L1 and CD80 to inhibit immune suppression.

In certain embodiments, the immune checkpoint inhibitor is the PD-L1 immune checkpoint inhibitor atezolizumab (Tecentriq®) administered in an effective amount for the treatment of metastatic bladder cancer, small cell lung cancer, metastatic melanoma, metastatic non-small cell lung cancer, or metastatic renal cell carcinoma. In some embodiments, atezolizumab is administered at 840 mg every 2 weeks, 1200 mg every 3 weeks, or 1680 mg every 4 weeks. In some embodiments, atezolizumab is administered prior to chemotherapy. In another aspect of this embodiment, the immune checkpoint inhibitor is durvalumab (Imfinzi®; AstraZeneca and MedImmune) administered in an effective amount for the treatment of small cell lung cancer, non-small cell lung cancer, or bladder cancer. In some embodiments, durvalumab is administered at 10 mg/kg every 2 weeks or 1500 mg every 4 weeks for patients that weigh more than 30 kg and 10 mg/kg every 2 weeks for patients who weigh less than 30 kg. In certain embodiments, the immune checkpoint inhibitor is the PD-L1 immune checkpoint inhibitor avelumab (Bavencio®; EMD Serono/Pfizer) administered in an effective amount for the treatment of Merkel cell carcinoma or urothelial carcinoma. In some embodiments, avelumab is administered at 800 mg every 2 weeks. In yet another aspect of the embodiment, the immune checkpoint inhibitor is KN035 (Alphamab) administered in an effective amount for the treatment of PD-L1 positive solid tumors.

### CTLA-4 Inhibitors

In certain aspects of this embodiment, the immune checkpoint inhibitor is a CTLA-4 immune checkpoint inhibitor that binds to CTLA-4 and inhibits immune suppression. CTLA-4 is a glycoprotein of the immunoglobulin superfamily (Brunet et al. Nature. 328(6127): 267-70(1987)) that suppresses T-cell responses through several convergent mechanisms (Guntermann et al. J Immunol. 168(9); 4420-9(2002); Kong et al. Nat Immunol. 15(5): 465-72(2014); Qureshi et al. J Biol Chem. 287(12): 9429-40(2012)). CTLA-4 inhibitors include, but are not limited to, ipilimumab, tremelimumab (AstraZeneca and MedImmune), AGEN1884 and AGEN2041 (Agenus).

In certain embodiments, the CTLA-4 immune checkpoint inhibitor is ipilimumab (Yervoy®) administered in an effective amount for the treatment of metastatic melanoma, adjuvant melanoma, or non-small cell lung cancer.

### LAG-3 Inhibitors

In another embodiment, the immune checkpoint inhibitor is a LAG-3 immune checkpoint inhibitor. LAG-3 (CD223) is encoded by the lymphocyte activating gene 3 (LAG-3) gene. LAG-3 is an immunoglobulin superfamily (IgSF) member that regulates numerous aspects of T cell function (Triebel et al. J Exp Med. 171:1393-405(1990)). LAG-3 is expressed on cell membranes of natural killer (NK) cells, B cells, tumor-infiltrating lymphocytes (TILs), T-cell subsets, and dendritic cells (DCs) (Triebel et al. J Exp Med. 171:1393-405(1990); KIsielow et al. Eur J Immunol. 35:2081-8(2005); Grosso et al. J Clin Invest. 117:3383-92(2009); Workman et al. J Immunol. 182:1885-91(2009); Andreae et al. J Immunol. 168:3874-80(2002)). LAG-3 binds with greater affinity than CD4 a nonholomorphic region of major histocompatibility complex 2 (MHC class II) (Baixeras et al. J Exp Med. 176:27-37(1992)). LAG-3 is an immune checkpoint receptor upregulated on both regulatory T cells (T_{regs}) and anergic T cells. Simultaneous blockade of LAG-3 receptors can result in an enhanced reversal of this anergic state relative to the blockade of one receptor alone (Grosso et al. J Immunol. 182:6659-69(2009)). The LAG-3/MHC class II complex leads to the downregulation of CD4+Ag-specific T cell clone proliferation and cytokine secretion (Huard et al. Eur J Immunol. 26:1180-6(1996)).

In some embodiments, the checkpoint inhibitor is a LAG-3 inhibitor that blocks the interaction of LAG-3 with MHC class II by binding to the LAG-3 receptor to inhibit immune suppression. Examples of LAG-3 immune checkpoint inhibitors include, but are not limited to, relatlimab (BMS 986016/Ono 4482; Bristol-Myers Squibb); tebotelimab (MGD013; Macrogenics); LAG525 (Immutep, Novartis); TSR-033 (Tesaro, GlaxoSmithKline); Eftilagimod alpha (IMP321, Immutep); REGN3767 (Regeneron); INCAGN02385 (Incyte); RO7247669 (Hoffman-LaRoche); Favezelimab (Merck Sharp & Dohme); CB213 (Crescendo Biologics); FS118 (F-star Therapeutics); SYM022 (Symphogen); GSK2831781 (GlaxoSmithKline); IBI323 (Innovent Biologics (Suzhou) Co. Ltd.); EMB-02 (Shanghai EpimAb Biotherapeutics Co., Ltd.); SNA03 (Microbio Group); and AVA021 (Avacta).

T Cell Immunoreceptor with Immunoglobulin and ITIM Domain (TIGIT) Inhibitors

In some embodiments, the immune checkpoint inhibitor is a T cell immunoreceptor with immunoglobulin and ITIM domain (TIGIT). TIGIT is a promising new target for cancer immunotherapy. TIGIT levels are upregulated in several immune cell subtypes, including activated T cells, natural killer cells, and regulatory T cells (T_{regs}). TIGIT binds to two ligands, CD155 (PVR) and CD112 (PVRL2, nectin-2), that are expressed by tumor cells and antigen-presenting cells (APCs) in the tumor microenvironment (Stanietsky et al. Proc Natl Acad Sci. 106:17858-63(2009)).

TIGIT (also referred to as WUCAM, Vstm3, VSIG9) is an Ig superfamily receptor which suppresses adaptive and innate immunity (Boles et al. Eur J Immunol. 39:695-703(2009)). TIGIT is a member of a complex regulatory network involving multiple inhibitory receptors (e.g., CD96/TACTILE, CD112R/PVRIG), one competing costimulatory receptor (DNAM-1/CD226), and multiple ligands (e.g., CD155 (PVR/NECL-5), CD112 (Nectin-2/PVRL2)) (Levin et al. Eur J Immunol. 41:902-15(2011); Bottino et al. J Exp Med. 198:557-67(2003); Seth et al. Biochem Biophys Res Commun. 364:959-65(2007); Zhu et al. J Exp Med 213:167-76(2016)).

TIGIT is expressed by activated CD4+ T cells and CD8+ T cells, natural killer (NK) cells, regulatory T cells (T_{regs}), and follicular T helper cells in humans (Joller et al. J Immunol. 186:1338-42(2011); Wu et al. Eur J Immunol. 46:1152-61(2016)), whereas TIGIT is weakly expressed by naïve T cells. In cancer, TIGIT is co-expressed with PD-1 on tumor antigen-specific CD8+ T cells and CD8+ tumor-infiltrating lymphocytes (TILs) in mice and humans (Chauvin et al. J Clin Invest. 125:2046-58(2015); Johnston et al. Cancer Cell. 26:923-37(2014)). TIGIT is highly expressed by T_{regs} in peripheral blood mononuclear cells (PBMCs) of healthy donors and patients with cancer and also upregulated in the TME (Joller et al. Immunity. 40:569-81(2014); Zhang et al. Blood. 122:2823-36(2013)). TIGIT is also co-expressed with other inhibitory receptors including TIM-3 and LAG-3 on exhausted CD8+ T cell subsets in tumors (Chauvin et al. J Clin Invest. 125:2046-58(2015); Johnston et al. Cancer Cell. 26:923-37(2014)).

In some embodiments, the immune checkpoint inhibitor is a TIGIT inhibitor that blocks the interaction of TIGIT and CD155 by binding to the TIGIT receptor to inhibit immune suppression. TIGIT inhibitors include, but are not limited to, Etigilimab (OMP-313M32; Oncomed Pharmaceuticals); Tiragolumab (MTIG7192A; RG6058; Roche/Genentech); (MK-7684; Merck); BMS-986207 (Bristol-Myers Squibb); AZD2936 Vibostolimab (AstraZeneca); ASP8374 (Astellas/Potenza Therapeutics); Domvanalimab (AB154; Arcus Biosciences); IBI939 (Innovent Biologics); Ociperlimab (BGB-A1217; BeiGene); EOS884448 (iTeos Therapeutics); SEA-TGT (Seattle Genetics); COM902 (Compugen); MPH-313 (Mereo Biopharma); M6223 (EMD Serono); HLX53 (Shanghai Henlius Biotech); JS006 (Junshi Bio); mAb-7 (Stanwei Biotech); SHR-1708 (Hengrui Medicine); BAT6005 (Bio-Thera Solutions); GS02 (Suzhou Zelgen/Qilu Pharma); RXI-804 (Rxi Pharmaceuticals); NB6253 (Northern Biologics); ENUM009 (Enumreal Biomedical); CASC-674 (Cascadian Therapeutics); AJUD008 (AJUD Biopharma); and AGEN1777 (Agenus, Bristol-Myers Squibb).

### T-Cell Immunoglobulin and Mucin Domain 3 (TIM-3) Inhibitors

In some embodiments, the immune checkpoint inhibitor is a T-cell immunoglobulin and mucin domain 3 (TIM-3) inhibitor. TIM-3 is an immunoglobulin (Ig) and mucin domain-containing cell surface-expressed molecule that was originally discovered as a cell surface marker specific to interferon gamma (IFN-γ) producing CD4+ T helper 1 (Th1) and CD8+ T cytotoxic 1 (Tc1) cells (Monney et al. Nature. 415:536-41(2002)). TIM-3 is co-regulated and co-expressed along with other immune checkpoint receptors (PD-1, LAG-3, and TIGIT) on CD4+ and CD8+ T cells (Chihara et al. Nature. 558:454-9(2018); DeLong et al. ImmunoHorizons. 3:13-25(2019)). TIM-3 expression is a marker of the substantially dysfunctional or terminally exhausted CD8+ T cell subsets in cancer (Fourcade et al. J Exp Med. 207:2175-86(2010); Sakuishi et al. J Exp Med. 207:2187-94(2010)). Several TIM-3 ligands have been identified including galectin-9, phosphatidylserine (PtdSer), high-mobility group protein B1 (HMGB1), and CEACAM-1.

In some embodiments, the immune checkpoint inhibitor is a TIM-3 inhibitor that blocks the interaction of TIM-3 and galectin-9, phosphatidylserine (PtdSer), high-mobility group protein B1 (HMGB1), and/or CEACAM-1 by binding to the TIM-3 receptor to inhibit immune suppression. TIM-3 inhibitors include, but are not limited to, Sabatolimab (MGB453; Novartis Pharmaceuticals); Cobolimab (TSR-022; Tesaro/GSK); RG7769 (Genentech); MAS-825 (Novartis); Sym023 (Symphogen A/S); BGBA425 (BeiGene); R07121661 (Hoffmann-La Roche); LY3321367 (Eli Lilly and Company); INCAGN02390 (Incyte Corporation); BMS-986258 (ONO7807, Bristol-Myers Squibb); AZD7789 (AstraZeneca); TQB2618 (Chia Tai Tianqing Pharmaceutical Group Co., Ltd.); and NB002 (Neologics Bioscience).

### Alternative Immune Checkpoint Inhibitors

In some embodiments, the patient is administered a B7-H3/CD276 immune checkpoint inhibitor such as enoblituzumab (MGA217, Macrogenics) MGD009 (Macrogenics), 131I-8H9/omburtamab (Y-mabs), and I-8H9/omburtamab (Y-mabs), an indoleamine 2,3-dioxygenase (IDO) immune checkpoint inhibitor such as Indoximod and INCB024360, a killer immunoglobulin-like receptors (KIRs) immune checkpoint inhibitor such as Lirilumab (BMS-986015), a carcinoembryonic antigen cell adhesion molecule (CEACAM) inhibitor (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof (as described in, e.g., US 2004/0047858, U.S. Pat. No. 7,132,255 and WO 99/052552). In other embodiments, the anti-CEACAM antibody binds to CEACAM-5 (as described in, e.g., Zheng et al. PloS One. 2:5(9). Pii: e12529 (DOI: 10:1371/journal.pone.0021146, 2010), or cross-reacts with CEACAM-1 and CEACAM-5 (as described in, e.g., WO 2013/054331 and US 2014/0271618).

In some embodiments, the patient is administered an ICI directed to CD47, including, but not limited to, Hu5F9-G4 (Stanford University/Forty Seven), TI-061 (Arch Oncology), TTI-622 (Trillum Therapeutics), TTI-621 (Trillum Therapeutics), SRF231 (Surface Oncology), SHR-1603 (Hengrui), OSE-172 (Boehringer Ingelheim/OSE Immunotherapeutics), NI-1701 (Novimmune TG Therapeutics), IBI188 (Innovent Biologics); CC-95251 (Celgene), CC-90002 (Celgene/Inibrx), AO-176 (Arch Oncology), ALX148 (ALX Oncology), IMM01 (ImmuneOnco Biopharma), IMM2504 (ImmuneOnco Biopharma), IMM2502 (ImmuneOnco Biopharma), IMM03 (ImmuneOnco Biopharma), IMC-002 (ImmuneOncia Therapeutics), IBI322 (Innovent Biologics), HMBD-004B (Hummingbird Bioscience), HMBD-004A (Hummingbird Bioscience), HLX24 (Henlius), FSI-189 (Forty Seven), DSP107 (KAHR Medical), CTX-5861 (Compass Therapeutics), BAT6004 (Bio-Thera), AUR-105 (Aurigene), AUR-104 (Aurigene), ANTI-CD47 (Biocad), ABP-500 (Abpro), ABP-160 (Abpro), TJC4 (I-MAB Biopharma), TJC4-CK (I-MAB Biopharma), SY102 (Saiyuan), SL-172154 (Shattuck Labs), PSTx-23 (Paradigm Shift Therapeutics), PDL1/CD47BsAb (Hanmi Pharmaceuticals), NI-1801 (Novimmune), MBT-001 (Morphiex), LYN00301 (LynkCell), and BH-29xx (Beijing Hanmi).

In some embodiments, the ICI is an inhibitor directed to CD39, including, but not limited to TTX-030 (Tizona Therapeutics), IPH5201 (Innate Pharma/AstraZeneca), SRF-617 (Surface Oncology), ES002 (Elpisciences), 9-8B (Igenica), and an antisense oligonucleotide (Secarna)

In some embodiments, the immune checkpoint inhibitor is an inhibitor directed to B and T lymphocyte attenuator molecule (BTLA) (as described in Zhang et al. Clin Exp Immunol. 163(1): 77-87(2011)), and TAB004/JS004 (Junshi Biosciences).

In some embodiments, the immune checkpoint inhibitor is a sialic acid-binding immunoglobulin-like lectin 15 (Siglec-15) inhibitor, including, but not limited to, NC318 (an anti-Siglec-15 mAb).

### Chemotherapeutic Agents

As contemplated herein, a CDK inhibitor described herein can be in combination with any standard chemotherapeutic agent treatment modality. In certain embodiments, a CDK inhibitor described herein can be in combination with any standard chemotherapeutic agent treatment modality and in further combination with an immune checkpoint inhibitor.

In certain embodiments, the chemotherapeutic agent is toxic to immune effector cells. In certain embodiments the chemotherapeutic agent inhibits cell growth. In certain embodiments, the cytotoxic chemotherapeutic agent administered is a DNA damaging chemotherapeutic agent. In certain embodiments, the chemotherapeutic agent is a protein synthesis inhibitor, a DNA-damaging chemotherapeutic, an alkylating agent, a topoisomerase inhibitor, an RNA synthesis inhibitor, a DNA complex binder, a thiolate alkylating agent, a guanine alkylating agent, a tubulin binder, DNA polymerase inhibitor, an anticancer enzyme, RAC1 inhibitor, thymidylate synthase inhibitor, oxazophosphorine compound, integrin inhibitor such as cilengitide, camptothecin or homocamptothecin, antifolate or a folate antimetabolite.

In some embodiments, the additional therapeutic agent is selected from elotuzumab, rituximab, lenalidomide, cytarabine, daratumumab, adalimumab, idealisib, gilteritinib, glasdegib, valaciclovir, acalabrutinib, ibrutinib, midostaurin, ruxolitinib, bortezomib, lapatinib, bendamstine, enzalutamide, azacitadine, obinutuzumab, decitabine, erdafitinib, or venetoclax.

In certain embodiments the additional therapeutic agent is trastuzumab. In certain embodiments the additional therapeutic agent is lapatinib. In certain embodiments the Compound I is dosed with 2, 3, or 4 additional therapeutic agents. In certain embodiments there are 2 additional therapeutic agents. In certain embodiments the two additional therapeutic agents are lapatinib and trastuzumab.

In certain embodiments the additional therapeutic agent is osimertinib mesylate (Tagrisso®).

In certain embodiments the additional therapeutic agent is alectinib (Alecensa®).

In certain embodiments the additional therapeutic agent is a MEK inhibitor.

In certain embodiments the additional therapeutic agent is an Androgen Receptor ligand.

In certain embodiments the additional therapeutic agent is a BTK inhibitor, for example but not limited to ibrutinib (Imbruvica®) or acalabrutinib (Calquence®).

In certain embodiments the additional therapeutic agents are a MEK inhibitor and a RAF inhibitor.

In certain embodiments the additional therapeutic agent is a RAF inhibitor.

In certain embodiments the additional therapeutic agent is regorafenib.

### Cytotoxic Chemotherapeutic Agents

Cytotoxic, DNA-damaging chemotherapeutic agents tend to be non-specific and, particularly at high doses, toxic to normal, rapidly dividing cells such as HSPC and immune effector cells. As used herein the term “DNA-damaging” chemotherapy or chemotherapeutic agent refers to treatment with a cytostatic or cytotoxic agent (i.e., a compound) to reduce or eliminate the growth or proliferation of undesirable cells, for example cancer cells, wherein the cytotoxic effect of the agent can be the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect. Such compounds include, but are not limited to, DNA damaging compounds that can kill cells. “DNA damaging” chemotherapeutic agents include, but are not limited to, alkylating agents, DNA intercalators, protein synthesis inhibitors, inhibitors of DNA or RNA synthesis, DNA base analogs, topoisomerase inhibitors, telomerase inhibitors, and telomeric DNA binding compounds. For example, alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylol melamine; nitrogen mustards such as chlorambucil, chlornaphazine, cyclophosphamide, estramustine, mechlorethamine, mechlorethamine oxide hydrochloride, melphalan, novembichine, phenesterine, prednimustine, trofosfamide, and uracil mustard; and nitroso ureas, such as carmustine, chlorozotocin, fotemustine, lomustine, nimustine, and ranimustine. Other DNA-damaging chemotherapeutic agents include daunorubicin, doxorubicin, idarubicin, epirubicin, mitomycin, and streptozocin. Chemotherapeutic antimetabolites include gemcitabine, mercaptopurine, thioguanine, cladribine, fludarabine phosphate, fluorouracil (5-FU), floxuridine, cytarabine, pentostatin, methotrexate, azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine β-1-D-arabinoside, diazooxynorleucine, dideoxynucleosides, 5-fluorodeoxycytidine, 5-fluorodeoxyuridine, and hydroxyurea.

Chemotherapeutic protein synthesis inhibitors include abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim.

Inhibitors of DNA synthesis, include alkylating agents such as dimethyl sulfate, nitrogen and sulfur mustards; intercalating agents, such as acridine dyes, actinomycins, anthracenes, benzopyrene, ethidium bromide, propidium diiodide-intertwining; and other agents, such as distamycin and netropsin. Topoisomerase inhibitors, such as irinotecan, teniposide, coumermycin, nalidixic acid, novobiocin, and oxolinic acid; inhibitors of cell division, including colcemide, mitoxantrone, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be used as the DNA damaging compound.

In certain embodiments the chemotherapeutic agent is a DNA complex binder such as camptothecin, or etoposide; a thiolate alkylating agent such as nitrosourea, BCNU, CCNU, ACNU, or fotesmustine; a guanine alkylating agent such as temozolomide, a tubulin binder such as vinblastine, vincristine, vinorelbine, vinflunine, cryptophycin 52, halichondrins, such as halichondrin B, dolastatins, such as dolastatin 10 and dolastatin 15, hemiasterlins, such as hemiasterlin A and hemiasterlin B, colchicine, combrestatins, 2-methoxyestradiol, E7010, paclitaxel, docetaxel, epothilone, discodermolide; a DNA polymerase inhibitor such as cytarabine; an anticancer enzyme such as asparaginase; a Rac1 inhibitor such as 6-thioguanine; a thymidylate synthase inhibitor such as capecitabine or 5-FU; a oxazophosphorine compound such as Cytoxan; a integrin inhibitor such as cilengitide; an antifolate such as pralatrexate; a folate antimetabolite such as pemetrexed; or a camptothecin or homocamptothecin such as diflomotecan.

In certain embodiments the topoisomerase inhibitor is a type I inhibitor. In another embodiment the topoisomerase inhibitor is a type II inhibitor.

Other DNA-damaging chemotherapeutic agents whose toxic effects can be mitigated by the presently disclosed selective CDK4/6 inhibitors include, but are not limited to, cisplatin, hydrogen peroxide, carboplatin, procarbazine, ifosfamide, bleomycin, plicamycin, taxol, transplatinum, thiotepa, oxaliplatin, and the like, and similar acting-type agents. In certain embodiments, the DNA damaging chemotherapeutic agent is selected from cisplatin, carboplatin, camptothecin, or etoposide.

Other suitable chemotherapeutic agents include, but are not limited to, radioactive molecules, toxins, also referred to as cytotoxins or cytotoxic agents, which includes any agent that is detrimental to the viability of cells, agents, and liposomes or other vesicles containing chemotherapeutic compounds. General anticancer pharmaceutical agents include: Vincristine (Oncovin®), liposomal vincristine (Marqibo®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Prednisone, and Dexamethasone (Decadron). Examples of additional suitable chemotherapeutic agents include but are not limited to 5-fluorouracil, dacarbazine, alkylating agents, anthramycin (AMC)), anti-mitotic agents, cis-dichlorodiamine platinum (II) (DDP) cisplatin), diamino dichloro platinum, anthracyclines, antibiotics, antimetabolites, asparaginase, BCG live (intravesical), bleomycin sulfate, calicheamicin, cytochalasin B, dactinomycin (formerly actinomycin), daunorubicin HCl, daunorubicin citrate, denileukin diftitox, dihydroxy anthracin dione, Docetaxel, doxorubicin HCl, E. coli L-asparaginase, Erwinia L-asparaginase, etoposide citrovorum factor, etoposide phosphate, gemcitabine HCL, idarubicin HCl, interferon α-2b, irinotecan HCl, maytansinoid, mechlorethamine HCl, melphalan HCl, mithramycin, mitomycin C, mitotane, polifeprosan 20 with carmustine implant, procarbazine HCl, streptozotocin, teniposide, thiotepa, topotecan HCl, valrubicin, vinblastine sulfate, vincristine sulfate, and vinorelbine tartrate.

Additional cytotoxic chemotherapeutic agents for use with the present invention include: epirubicin, abraxane, taxotere, epothilone, tafluposide, vismodegib, azacytidine, doxifluridine, vindesine, and vinorelbine.

In certain embodiments the chemotherapeutic agent is not an aromatase inhibitor. In certain embodiments the chemotherapeutic agent is not a steroid. In certain embodiments the chemotherapeutic agent is not a BCR-ABL inhibitor.

In certain embodiments the chemotherapeutic agent is a DNA complex binder. In certain embodiments the chemotherapeutic agent is a tubulin binder. In certain embodiments the chemotherapeutic agent is an alkylating agent. In certain embodiments the chemotherapeutic agent is a thiolate alkylating agent.

### Additional Chemotherapeutic Agents

Additional chemotherapeutic agents that may be used in the methods as described herein may include 2-methoxyestradiol or 2ME2, finasunate, etaracizumab (MEDI-522), HLL1, huN901-DM1, atiprimod, saquinavir mesylate, ritonavir, nelfinavir mesylate, indinavir sulfate, plitidepsin, P276-00, tipifarnib, lenalidomide, thalidomide, pomalidomide, simvastatin, and celecoxib. Chemotherapeutic agents useful in the present invention include, but are not limited to, Trastuzumab (Herceptin®), Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Targretin®), Alitretinoin (Panretin®), Tretinoin (Vesanoid®), Carfilzomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

Additional chemotherapeutic agents contemplated include, but are not limited to, a calcineurin inhibitor, e.g. a cyclosporin or an ascomycin, e.g. Cyclosporin A (Neoral®), FK506 (tacrolimus), pimecrolimus, a mTOR inhibitor, e.g. rapamycin or a derivative thereof, e.g. Sirolimus (Rapamune®), Everolimus (Certican®), temsirolimus, zotarolimus, biolimus-7, biolimus-9, a rapalog, e.g. ridaforolimus, campath 1H, a SIP receptor modulator, a dual mTORC1 and mTORC2 inhibitor, eg. Vistusertib (AZD2014), e.g. fingolimod or an analogue thereof, an anti IL-8 antibody, mycophenolic acid or a salt thereof, e.g. sodium salt, or a prodrug thereof, e.g. Mycophenolate Mofetil (CellCept®), OKT3 (Orthoclone OKT3®), Prednisone, ATGAM®, Thymoglobulin®, Brequinar Sodium, OKT4, T10B9.A-3A, 33B3.1, 15-deoxyspergualin, tresperimus, Leflunomide Arava®, anti-CD25, anti-IL2R, Basiliximab (Simulect®), Daclizumab (Zenapax®), mizoribine, dexamethasone, ISAtx-247, SDZ ASM 981 (pimecrolimus, Elidel®), Abatacept, belatacept, LFA31g, etanercept (sold as Enbrel® by ImmuneXcite), adalimumab (Humira®), infliximab (Remicade®), an anti-LFA-1 antibody, natalizumab (Antegren®), Enlimomab, gavilimomab, Golimumab, antithymocyte immunoglobulin, siplizumab, Alefacept, efalizumab, Pentasa, mesalazine, asacol, codeine phosphate, benorylate, fenbufen, naprosyn, diclofenac, etodolac, indomethacin, dasatinib (Sprycel®) nilotinib (Tasigna®), bosutinib (Bosulif®), Imatinib mesylate (Gleevec®) and ponatinib (Iclusig™) amifostine, dolasetron mesylate, dronabinol, epoetin-α, etidronate, filgrastim, fluconazole, goserelin acetate, gramicidin D, granisetron, leucovorin calcium, lidocaine, Mesna, ondansetron HCl, pilocarpine HCl, porfimer sodium, vatalanib, 1-dehydrotestosterone, allopurinol sodium, Betamethasone, sodium phosphate and betamethasone acetate, calcium leucovorin, conjugated estrogens, Dexrazoxane, Dibromomannitol, esterified estrogens, estradiol, estramustine phosphate sodium, ethinyl estradiol, flutamide, folinic acid, glucocorticoids, leuprolide acetate, levamisole HCl, medroxyprogesterone acetate, megestrol acetate, methyltestosterone, nilutamide, octreotide acetate, pamidronate disodium, procaine, propranolol, testolactone, tetracaine, toremifene citrate, and sargramostim.

In certain embodiments the chemotherapeutic agent is an estrogen receptor ligands such as tamoxifen, raloxifene, fulvestrant, anordrin, bazedoxifene, broparestriol, chlorotrianisene, clomiphene citrate, cyclofenil, lasofoxifene, ormeloxifene, or toremifene; an androgen receptor ligand such as bicalutamide, enzalutamide, apalutamide, cyproterone acetate, chlormadinone acetate, spironolactone, canrenone, drospirenone, ketoconazole, topilutamide, abiraterone acetate, or cimetidine; an aromatase inhibitor such as letrozole, anastrozole, or exemestane; an anti-inflammatory such as prednisone; an oxidase inhibitor such as allopurinol; an anticancer antibody; an anticancer monoclonal antibody; an antibody against CD40 such as lucatumumab or dacetuzumab; an antibody against CD20 such as rituximab; an antibody that binds CD52 such as alemtuzumab; an antibody that binds integrin such as volociximab or natalizumab; an antibody against interleukin-6 receptor such as tocilizumab; an interleukin-2 memetic such as aldesleukin; an antibody that targets IGF1 like figitumumab; an antibody that targets DR4 such as mapatumumab; an antibody that targets TRAIL-R2 such as lexatumumab or dulanermin; a fusion protein such as atacicept; a B cell inhibitor such as atacicept; a proteasome inhibitor such as carfilzomib, bortezomib, or marizomib; a HSP90 inhibitor such as tanespimycin; a HDAC inhibitor such as vorinostat, belinostat or panobinostat; a MAPK ligand such as talmapimod; a PKC inhibitor such as enzastaurin; a HER2 receptor ligand such as trastuzumab, lapatinib, or pertuzumab; an EGFR inhibitor such as gefitinib, erlotinib, cetuximab, panitumumab, or vandetanib; a natural product such as romidepsin; a retinoid such as bexarotene, tretinoin, or alitretinoin; a receptor tyrosine kinase (RTK) inhibitor such as sunitinib, regorafenib, or pazopanib; or a VEGF inhibitor such as ziv-aflibercept, bevacizumab or dovitinib.

In certain embodiments, the combinations of a CDK4/6 inhibitor, chemotherapeutic agent, and immune checkpoint inhibitor is further combined with the use of hematopoietic growth factors including, but not limited to, granulocyte colony stimulating factor (G-CSF, for example, sold as Neupogen® (filgrastim), Neulasta® (peg-filgrastim), or lenograstim), granulocyte-macrophage colony stimulating factor (GM-CSF, for example sold as molgramostim and sargramostim (Leukine®)), M-CSF (macrophage colony stimulating factor), Thrombopoietin (megakaryocyte growth development factor (MGDF), for example sold as Romiplostim® and Eltrombopag®) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-α as Darbepoetin, Epocept, Nanokine, Epofit, Epogen, Eprex, and Procrit; epoetin-β sold as for example NeoRecormon, Recormon and Micera), epoetin-delta (sold as for example Dynepo), epoetin-omega (sold as for example Epomax), epoetin zeta (sold as for example Silapo and Retacrit) as well as for example Epocept, Epotrust, Erypro Safe, Repoitin, Vintor, Epofit, Erykine, Wepox, Espogen, Relipoietin, Shanpoietin, Zyrop and EPIAO).

In certain embodiments, the administration of Compound I or pharmaceutically acceptable salt thereof or morphic form as described herein using a method described herein is combined with the use of a hematopoietic growth factor including, but not limited to, granulocyte colony stimulating factor (G-CSF), granulocyte-macrophage colony stimulating factor (GM-CSF), thrombopoietin, interleukin (IL)-12, steel factor, and erythropoietin (EPO), or their derivatives. In certain embodiments, the compound is administered prior to administration of the hematopoietic growth factor. In certain embodiments, the hematopoietic growth factor administration is timed so that the compound's effect on HSPCs has dissipated.

Additional active compounds contemplated herein, particularly in the treatment of abnormal tissue of the female reproductive system such as breast, ovarian, endometrial, or uterine cancer include a CDK2 inhibitor described herein in combination with an estrogen inhibitor including but not limited to a SERM (selective estrogen receptor modulator), a SERD (selective estrogen receptor degrader), a complete estrogen receptor degrader, or another form of partial or complete estrogen antagonist. Partial anti-estrogens include raloxifene and tamoxifen retain some estrogen-like effects. Complete anti-estrogens include fulvestrant. Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, WO2017/100712, WO2017/100715, WO2018/081168, and WO2018/148576 assigned to G1 Therapeutics, and U.S. Pat. Nos. 9,078,871, 8,853,423, and 8,703,810, as well as US 2015/0005286, WO 2014/205136, and WO 2014/205138. Additional non-limiting examples of anti-estrogen compounds include: SERMS such as anordrin, arzoxifene, bazedoxifene, broparestriol, clomiphene citrate, cyclofenil, droloxifene, endoxifen, idoxifene, lasofoxifene, ormeloxifene, pipendoxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, delmadinone acetate, dydrogesterone, medroxyprogesterone acetate, megestrol acetate, nomegestrol acetate, norethisterone acetate, progesterone, and spironolactone. Additional non-limiting examples of anti-estrogen compounds include: SERDS such as fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, or ZN-c5.

In certain embodiments the SERD compound of the Formula described in WO 2017/100712.

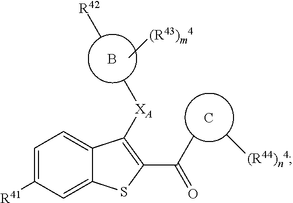

or a pharmaceutically acceptable salt thereof.

### Wherein:

m⁴ is 0, 1, 2, 3, or 4;
n⁴ is 0, 1, 2, 3, or 4;
X_{A} is selected from —O—, —CH₂—, —S—, —NH—, —Nme-, —CF₂—, and C₃cycloalkyl;
Ring B is phenyl, naphthyl, quinolinyl, 5- or 6-membered monocyclic heteroaryl or 7-, 8-, 9- or 10 membered bicyclic heterocycle;
Ring C is phenyl, thienyl, 5- or 6-membered monocyclic heteroaryl or 7-, 8-, 9- or 10-membered bicyclic heterocycle;
R⁴¹ is selected from hydroxyl, hydrogen, halogen, —O(C₁-C₆ alkyl), —OC(O)(C₁-C₆ alkyl), —OC(O)C₆H₅, —OC(O)O(C₁-C₆ alkyl), —OC(O)OC₆H₅ and —OSO₂(C₂-C₆ alkyl);
R⁴² is selected from —CH═CHCOOH, —NH(CO)COOH, —COOH, —C₂-C₆alkenylene-COOH and —C₂-C₆alkynylene-COOH;
R⁴³ is independently selected at each occurrence from hydrogen, halogen, —CN, —NO₂, —C₁-C₆alkyl and —C₁-C₆fluoroalkyl; and
R⁴⁴ is independently selected at each occurrence from hydrogen, halogen, hydroxyl, —C₁-C₆alkyl, —C₁-C₆fluoroalkyl, —CN, —O(C₁-C₆alkyl), and —O(C₁-C₆fluoroalkyl).

Non-limiting examples of SERDS for use in the present invention include:

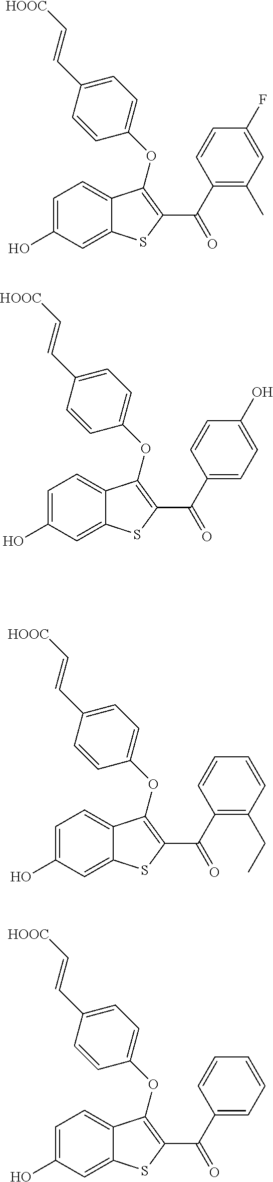 [SMILES: Cc1cc(F)ccc1C(=O)c1sc2cc(O)ccc2c1Oc1ccc(/C=C/C(=O)O)cc1 ; O=C(O)/C=C/c1ccc(Oc2c(C(=O)c3ccc(O)cc3)sc3cc(O)ccc23)cc1 ; CCc1ccccc1C(=O)c1sc2cc(O)ccc2c1Oc1ccc(/C=C/C(=O)O)cc1 ; O=C(O)/C=C/c1ccc(Oc2c(C(=O)c3ccccc3)sc3cc(O)ccc23)cc1]

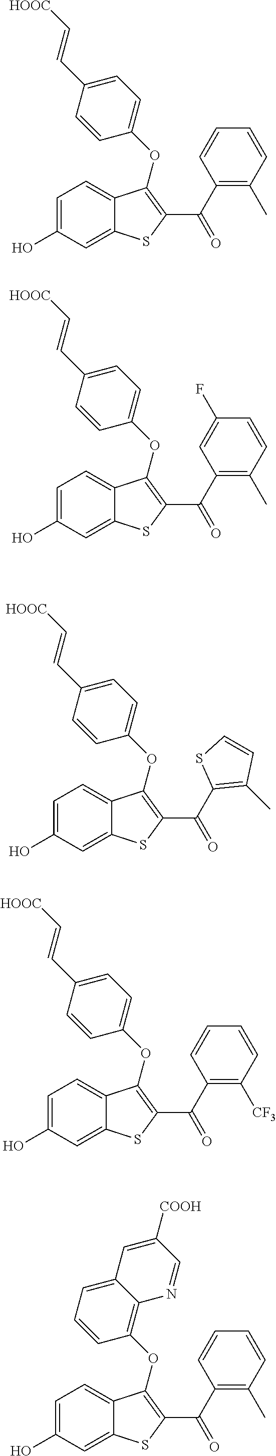 [SMILES: Cc1ccccc1C(=O)c1sc2cc(O)ccc2c1Oc1ccc(/C=C/C(=O)O)cc1 ; Cc1ccc(F)cc1C(=O)c1sc2cc(O)ccc2c1Oc1ccc(/C=C/C(=O)O)cc1 ; Cc1ccsc1C(=O)c1sc2cc(O)ccc2c1Oc1ccc(/C=C/C(=O)O)cc1 ; O=C(O)/C=C/c1ccc(Oc2c(C(=O)c3ccccc3C(F)(F)F)sc3cc(O)ccc23)cc1 ; Cc1ccccc1C(=O)c1sc2cc(O)ccc2c1Oc1cccc2cc(C(=O)O)cnc12]

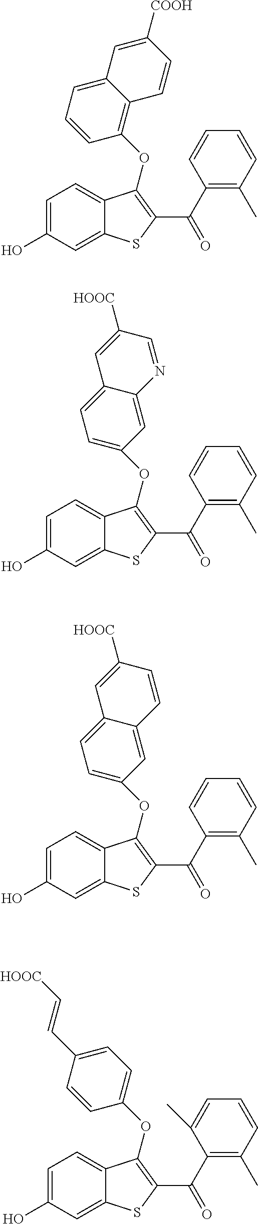 [SMILES: Cc1ccccc1C(=O)c1sc2cc(O)ccc2c1Oc1cccc2cc(C(=O)O)ccc12 ; Cc1ccccc1C(=O)c1sc2cc(O)ccc2c1Oc1ccc2cc(C(=O)O)cnc2c1 ; Cc1ccccc1C(=O)c1sc2cc(O)ccc2c1Oc1ccc2cc(C(=O)O)ccc2c1 ; Cc1cccc(C)c1C(=O)c1sc2cc(O)ccc2c1Oc1ccc(/C=C/C(=O)O)cc1]

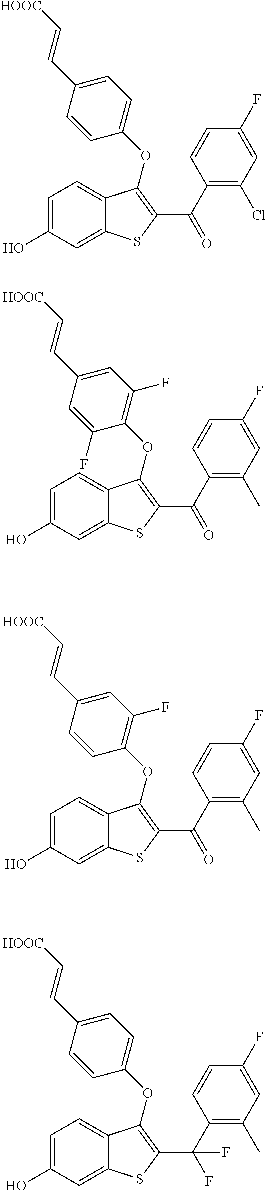 [SMILES: O=C(O)/C=C/c1ccc(Oc2c(C(=O)c3ccc(F)cc3Cl)sc3cc(O)ccc23)cc1 ; Cc1cc(F)ccc1C(=O)c1sc2cc(O)ccc2c1Oc1c(F)cc(/C=C/C(=O)O)cc1F ; Cc1cc(F)ccc1C(=O)c1sc2cc(O)ccc2c1Oc1ccc(/C=C/C(=O)O)cc1F ; Cc1cc(F)ccc1C(F)(F)c1sc2cc(O)ccc2c1Oc1ccc(/C=C/C(=O)O)cc1]

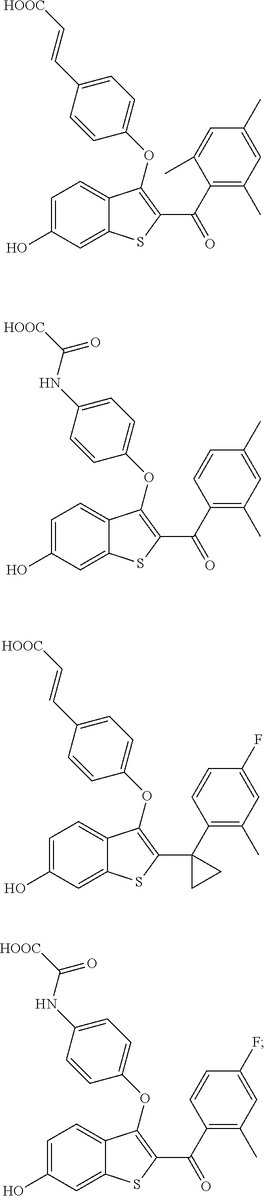 [SMILES: Cc1cc(C)c(C(=O)c2sc3cc(O)ccc3c2Oc2ccc(/C=C/C(=O)O)cc2)c(C)c1 ; Cc1ccc(C(=O)c2sc3cc(O)ccc3c2Oc2ccc(NC(=O)C(=O)O)cc2)c(C)c1 ; Cc1cc(F)ccc1C1(c2sc3cc(O)ccc3c2Oc2ccc(/C=C/C(=O)O)cc2)CC1 ; Cc1cc(F)ccc1C(=O)c1sc2cc(O)ccc2c1Oc1ccc(NC(=O)C(=O)O)cc1]

or a pharmaceutically acceptable salt thereof.

In certain embodiments the SERD is:

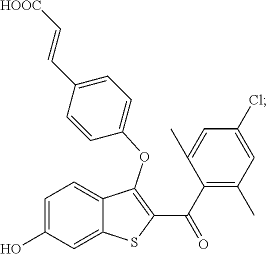 [SMILES: Cc1cc(Cl)cc(C)c1C(=O)c1sc2cc(O)ccc2c1Oc1ccc(/C=C/C(=O)O)cc1]

or a pharmaceutically acceptable salt thereof.

In certain embodiments the SERD is:

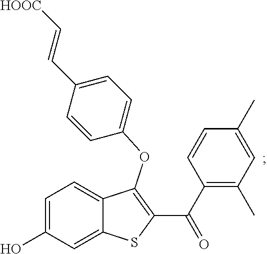 [SMILES: Cc1ccc(C(=O)c2sc3cc(O)ccc3c2Oc2ccc(/C=C/C(=O)O)cc2)c(C)c1]

or a pharmaceutically acceptable salt thereof.

In certain embodiments the SERD is:

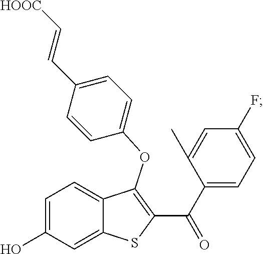 [SMILES: Cc1cc(F)ccc1C(=O)c1sc2cc(O)ccc2c1Oc1ccc(/C=C/C(=O)O)cc1]

or a pharmaceutically acceptable salt thereof.

Additional chemotherapeutic agents contemplated herein, particularly in the treatment of abnormal tissue of the male reproductive system such as prostate or testicular cancer, include, but are not limited to, an androgen (such as testosterone) inhibitor including but not limited to a selective androgen receptor modulator, a selective androgen receptor degrader, a complete androgen receptor degrader, or another form of partial or complete androgen antagonist. In certain embodiments, the prostate or testicular cancer is androgen-resistant. Non-limiting examples of anti-androgen compounds are provided in WO 2011/156518 and U.S. Pat. Nos. 8,455,534 and 8,299,112. Additional non-limiting examples of anti-androgen compounds include: chlormadinone acetate, spironolactone, canrenone, drospirenone, ketoconazole, topilutamide, abiraterone acetate, and cimetidine.

The chemotherapeutic agent may include a kinase inhibitor, including but not limited to a phosphoinositide 3-kinase (PI3K) inhibitor, a Bruton's tyrosine kinase (BTK) inhibitor, or a spleen tyrosine kinase (Syk) inhibitor, or a combination thereof.

PI3k inhibitors are well known. Examples of PI3 kinase inhibitors include but are not limited to Wortmannin, demethoxyviridin, perifosine, idelalisib, pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen(S)-methylphosphonate; or Methyl(oxo) {[(2R)-1-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((+)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino) quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-(5-[2,6-Di(4-morpholinyl)-4-alpyridin-6-yl)-N-tert-butylpyridine-3-sulfonamide), buparlisib pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), XL-765 (N-(3-(N-(3-(3,5-dimethoxyphenylamino) quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl]acetate (also known as sonolisib)), and the structure described in WO2014/071109 having the formula:

BTK inhibitors are well known. Examples of BTK inhibitors include ibrutinib (also known as PCI-32765) (Imbruvica™) (1-[(3R)-3-[4-amino-3-(4-phenoxy-phenyl)pyrazolo[3,4-d]pyrimidin-1-yl]piperidin-1-yl]prop-2-en-1-one), acalabrutinib (Calquence®), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), dasatinib ([N-(2-chloro-6-methylphenyl)-2-(6-(4-(2-hydroxyethyl)piperazin-1-yl)-2-methylpyrimidin-4-ylamino) thiazole-5-carboxamide], LFM-A13 (alpha-cyano-beta-hydroxy-beta-methyl-N-(2,5-ibromophenyl)propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino) phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), BGB-3111, and other molecules capable of inhibiting BTK activity, for example those BTK inhibitors disclosed in Akinleye et al, Journal of Hematology & Oncology, 2013, 6:59, the entirety of which is incorporated herein by reference.

Syk inhibitors are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevec; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

The chemotherapeutic agent can also be a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio) butan-2-yl)amino)-3 ((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen).

Additional chemotherapeutic agents for use in the methods contemplated herein include, but are not limited to, midazolam, MEK inhibitors, RAS inhibitors, ERK inhibitors, ALK inhibitors, HSP inhibitors (for example, HSP70 and HSP 90 inhibitors, or a combination thereof), RAF inhibitors, apoptotic compounds, topoisomerase inhibitors, AKT inhibitors, including but not limited to, MK-2206, GSK690693, Perifosine, (KRX-0401), GDC-0068, Triciribine, AZD5363, Honokiol, PF-04691502, and Miltefosine, or FLT-3 inhibitors, including but not limited to, P406, Dovitinib, Quizartinib (AC220), Amuvatinib (MP-470), Tandutinib (MLN518), ENMD-2076, and KW-2449, or combinations thereof. Examples of MEK inhibitors include but are not limited to trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino) isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide). Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol.

Known ERK inhibitors include SCH772984 (Merck/Schering-Plough), VTX-Ile (Vertex), DEL-22379, Ulixertinib (BVD-523, VRT752271), GDC-0994, FR 180204, XMD8-92, and ERK5-IN-1.

Raf inhibitors are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), and Sorafenib N-Oxide (4-[4-[[[[4-Chloro-3 (trifluoroMethyl)phenyl]amino]carbonyl]amino]phenoxy]-N-Methyl-2pyridinecarboxaMide 1-Oxide).

Known topoisomerase I inhibitors useful in the present invention include(S)-10-[(dimethylamino)methyl]-4-ethyl-4,9-dihydroxy-1H-pyrano[3′,4′:6,7]indolizino[1,2-b]quinoline-3,14(4H, 12H)-dione monohydrochloride (topotecan), (S)-4-ethyl-4-hydroxy-1H-pyrano[3′,4′:6,7]indolizino[1,2-b]quinoline-3,14-(4H, 12H)-dione (camptothecin), (1S,9S)-1-Amino-9-ethyl-5-fluoro-1,2,3,9,12,15-hexahydro-9-hydroxy-4-methyl-10H,13H-benzo (de) pyrano(3′,4′:6,7)indolizino (1,2-b)quinoline-10,13-dione (exatecan), (7-(4-methylpiperazinomethylene)-10,11-ethylenedioxy-20 (S)-camptothecin (lurtotecan), or(S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate (irinotecan), (R)-5-ethyl-9,10-difluoro-5-hydroxy-4,5-dihydrooxepino[3′,4′:6,7]indolizino[1,2-b]quinoline-3,15(1H,13H)-dione (diflomotecan), (4S)-11-((E)-((1,1-Dimethylethoxy)imino)methyl)-4-ethyl-4-hydroxy-1,12-dihydro-14H-pyrano(3′,4′:6,7)indolizino (1,2-b)quinoline-3,14(4H)-dione (gimatecan), (S)-8-ethyl-8-hydroxy-15-((4-methylpiperazin-1-yl)methyl)-11,14-dihydro-2H-[1,4]dioxino[2,3-g]pyrano[3′,4′:6,7]indolizino[1,2-b]quinoline-9,12(3H,8H)-dione (lurtotecan), (4S)-4-Ethyl-4-hydroxy-11-[2-[(1-methylethyl)amino]ethyl]-1H-pyrano[3,4:6,7]indolizino[1,2-b]quinoline-3,14(4H,12H)-dione (belotecan), 6-((1,3-dihydroxypropan-2-yl)amino)-2,10-dihydroxy-12-((2R,3R,4S,5S,6R)-3,4,5-trihydroxy-6-(hydroxymethyl)tetrahydro-2H-pyran-2-yl)-12,13-dihydro-5H-indolo[2,3-a]pyrrolo[3,4-c]carbazole-5,7(6H)-dione (edotecarin), 8,9-dimethoxy-5-(2-N,N-dimethylaminoethyl)-2,3-methylenedioxy-5H-dibenzo(c,h)(1,6) naphthyridin-6-one (topovale), benzo[6,7]indolizino[1,2-b]quinolin-11(13H)-one (rosettacin), (S)-4-ethyl-4-hydroxy-11-(2-(trimethylsilyl)ethyl)-1H-pyrano[3′,4′:6,7]indolizino[1,2-b]quinoline-3,14(4H,12H)-dione (cositecan), tetrakis{(4S)-9-[([1,4′-bipiperidinyl]-1′-carbonyl)oxy]-4,11-diethyl-3,14-dioxo-3,4,12,14-tetrahydro-1H-pyrano[3′,4′:6,7]indolizino[1,2-b]quinolin-4-yl}N,N′,N″,N′″-{methanetetrayltetrakis[methylenepoly(oxyethylene)oxy(1-oxoethylene)]}tetraglycinate tetrahydrochloride (etirinotecan pegol), 10-hydroxy-camptothecin (HOCPT), 9-nitrocamptothecin (rubitecan), SN38 (7-ethyl-10-hydroxycamptothecin), and 10-hydroxy-9-nitrocamptothecin (CPT109), (R)-9-chloro-5-ethyl-5-hydroxy-10-methyl-12-((4-methylpiperidin-1-yl)methyl)-4,5-dihydrooxepino[3′,4′:6,7]indolizino[1,2-b]quinoline-3,15(1H,13H)-dione (elmotecan).

In certain embodiments, the chemotherapeutic agent is not an aromatase inhibitor. In certain embodiments, the chemotherapeutic agent is not an estrogen or androgen receptor agonist or antagonist.

### Growth Factors

In certain embodiments, the combination of Compound I, or a pharmaceutically acceptable salt thereof or morphic form as described herein, a CDK4/6 inhibitor, chemotherapeutic agent, and/or checkpoint inhibitor is further combined with the use of hematopoietic growth factors including, but not limited to, granulocyte colony stimulating factor (G-CSF, for example, sold as Neupogen (filgrastim), Neulasta (peg-filgrastim), or lenograstim), granulocyte-macrophage colony stimulating factor (GM-CSF, for example sold as molgramostim and sargramostim (Leukine)), M-CSF (macrophage colony stimulating factor), Thrombopoietin (megakaryocyte growth development factor (MGDF), for example sold as Romiplostim and Eltrombopag) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-α as Darbepoetin, Epocept, Nanokine, Epofit, Epogen, Eprex, and Procrit; epoetin-β sold as for example NeoRecormon, Recormon and Micera), epoetin-delta (sold as for example Dynepo), epoetin-omega (sold as for example Epomax), epoetin zeta (sold as for example Silapo and Retacrit) as well as for example Epocept, Epotrust, Erypro Safe, Repoitin, Vintor, Epofit, Erykine, Wepox, Espogen, Relipoietin, Shanpoietin, Zyrop and EPIAO).

### CDK4/6 Inhibitors

The present invention also provides advantageous methods to treat a patient with a selective CDK4/6 inhibitor-resistant cancer, which includes administering an effective amount of Compound I, or a pharmaceutically acceptable composition, salt, or morphic form as described herein or isotopic analog thereof. In certain aspects, Compound I is used to treat a patient with a cancer intrinsically resistant to selective CDK4/6 inhibition. In certain aspects, Compound I, is used to treat a patient with a cancer that has acquired resistance to one or more selective CDK4/6 inhibitors. In certain embodiments, the cancer has progressed following a prior regimen comprising a CDK4/6 inhibitor. In certain aspects, Compound I, is administered in combination with a selective CDK4/6 inhibitor to a patient with a selective CDK4/6 inhibition responsive cancer in order to extend the therapeutic effectiveness of the selective CDK 4/6 inhibitor. In certain aspects, Compound I, is administered in combination with a selective CDK 4/6 inhibitor to a patient with a selective CDK4/6 inhibition responsive cancer, wherein the patient is selective CDK4/6 inhibitor naïve. Selective CDK4/6 inhibitors for use in combination with Compound I include, but are not limited to palbociclib, abemaciclib, ribociclib, trilaciclib, SHR6390 (dalpiciclib), and lerociclib. In alternative embodiments, the CDK4/6 inhibitor for use in combination with Compound I is selected from BPI-16350, narazaciclib (ON-123300), FLX-925 (AMG-925), UCT-03-008, GLR2007, birociclib (XZP-3287), LY5219, PF-07220060, or ON-123300

In certain embodiments, the selective CDK4/6 inhibitor is palbociclib:

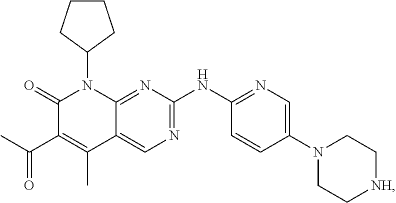 [SMILES: CC(=O)c1c(C)c2cnc(Nc3ccc(N4CCNCC4)cn3)nc2n(C2CCCC2)c1=O]

or a pharmaceutically acceptable salt thereof.

In certain embodiments, the selective CDK4/6 inhibitor is abemaciclib:

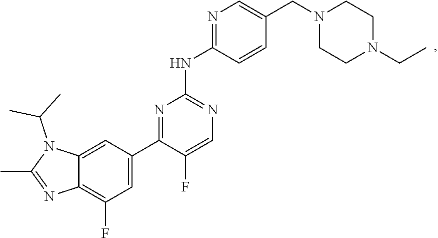 [SMILES: CCN1CCN(Cc2ccc(Nc3ncc(F)c(-c4cc(F)c5nc(C)n(C(C)C)c5c4)n3)nc2)CC1]

or a pharmaceutically acceptable salt thereof.

In certain embodiments, the selective CDK4/6 inhibitor is ribociclib:

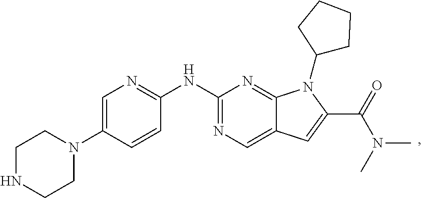 [SMILES: CN(C)C(=O)c1cc2cnc(Nc3ccc(N4CCNCC4)cn3)nc2n1C1CCCC1]

or a pharmaceutically acceptable salt thereof.

In certain embodiments, the selective CDK4/6 inhibitor is lerociclib:

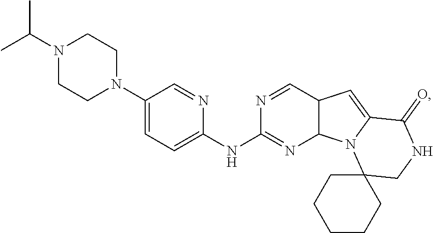 [SMILES: CC(C)N1CCN(c2ccc(NC3=NC4C(C=N3)C=C3C(=O)NCC5(CCCCC5)N34)nc2)CC1]

or a pharmaceutically acceptable salt thereof.

In certain embodiments, the selective CDK4/6 inhibitor is trilaciclib:

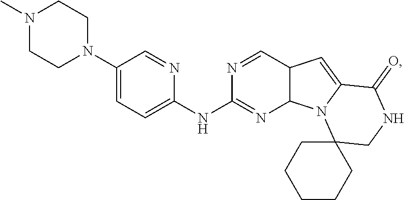 [SMILES: CN1CCN(c2ccc(NC3=NC4C(C=N3)C=C3C(=O)NCC5(CCCCC5)N34)nc2)CC1]

or a pharmaceutically acceptable salt thereof.

In certain embodiments, the selective CDK4/6 inhibitor is SHR6390 (dalpiciclib):

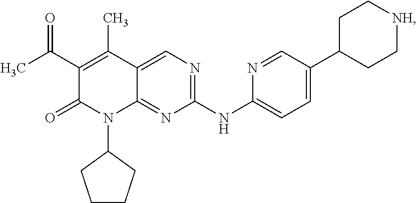 [SMILES: CC(=O)c1c(C)c2cnc(Nc3ccc(C4CCNCC4)cn3)nc2n(C2CCCC2)c1=O]

or a pharmaceutically acceptable salt thereof.

In certain embodiments, the selective CDK4/6 inhibitor is BPI-16350:

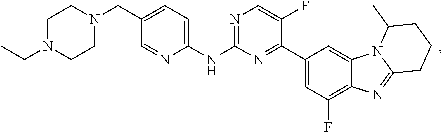 [SMILES: CCN1CCN(Cc2ccc(Nc3ncc(F)c(-c4cc(F)c5nc6n(c5c4)C(C)CCC6)n3)nc2)CC1]

or a pharmaceutically acceptable salt thereof.

In certain embodiments, the selective CDK4/6 inhibitor is narazaciclib (ON-123300):

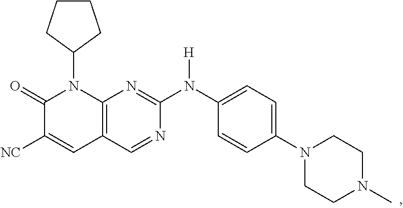 [SMILES: CN1CCN(c2ccc(Nc3ncc4cc(C#N)c(=O)n(C5CCCC5)c4n3)cc2)CC1]

or a pharmaceutically acceptable salt thereof.

In certain embodiments, the selective CDK4/6 inhibitor is FLX-925 (AMG-925):

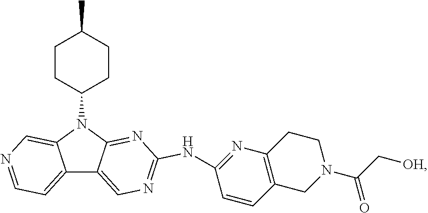 [SMILES: C[C@H]1CC[C@H](n2c3cnccc3c3cnc(Nc4ccc5c(n4)CCN(C(=O)CO)C5)nc32)CC1]

or a pharmaceutically acceptable salt thereof.

In certain embodiments, the selective CDK4/6 inhibitor is UCT-03-008, or a pharmaceutically acceptable salt thereof.

In certain embodiments, the selective CDK4/6 inhibitor is GLR2007:

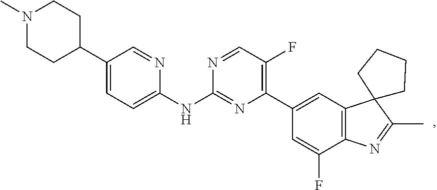

or a pharmaceutically acceptable salt thereof.

In certain embodiments, the selective CDK4/6 inhibitor is birociclib (XZP-3287), or a pharmaceutically acceptable salt thereof.

In certain embodiments, the selective CDK4/6 inhibitor is LY5219, or a pharmaceutically acceptable salt thereof.

In certain embodiments, the selective CDK4/6 inhibitor is PF-07220060:

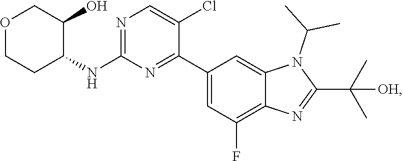 [SMILES: CC(C)n1c(C(C)(C)O)nc2c(F)cc(-c3nc(N[C@@H]4CCOC[C@H]4O)ncc3Cl)cc21]

or a pharmaceutically acceptable salt thereof.

In certain embodiments, the selective CDK4/6 inhibitor is ON-123300:

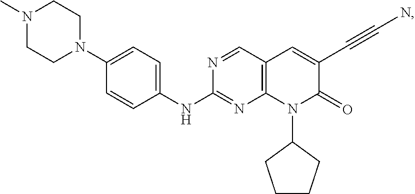

or a pharmaceutically acceptable salt thereof.

In certain embodiments, the selective CDK4/6 inhibitor is selected from an inhibitor described in, for example, U.S. Pat. Nos. 8,822,683; 8,598,197; 8,598,186; 8,691,830; 8,829,102; 8,822,683; 9,102,682; 9,499,564; 9,481,591; and U.S. Pat. No. 9,260,442, filed by Tavares and Strum and assigned to G1 Therapeutics describe a class of N-(heteroaryl)-pyrrolo[3,2-d]pyrimidin-2-amine cyclin dependent kinase inhibitors including those of the formula (with variables as defined therein):

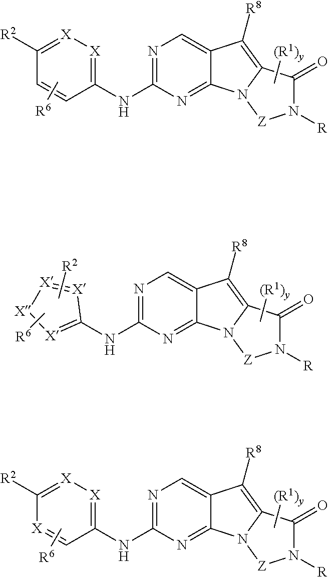

In certain embodiments, the selective CDK4/6 inhibitor is selected from an inhibitor described in, for example, U.S. Pat. Nos. 9,464,092, 9,487,530, and 9,527,857 which are also assigned to G1 Therapeutics describe the use of the above pyrimidine-based agents in the treatment of cancer.

In certain embodiments, the selective CDK4/6 inhibitor is selected from an inhibitor described in, for example, WO 2013/148748 (U.S. Ser. No. 61/617,657) titled “Lactam Kinase Inhibitors”, WO 2013/163239 (U.S. Ser. No. 61/638,491) titled “Synthesis of Lactams” and WO 2015/061407 filed by Tavares and also assigned to G1 Therapeutics describes the synthesis of N-(heteroaryl)-pyrrolo[3,2-d]pyrimidin-2-amines and their use as lactam kinase inhibitors.

In certain embodiments, the selective CDK4/6 inhibitor is selected from an inhibitor described in, for example, WO 2014/144326 filed by Strum et al. and assigned to G1 Therapeutics describes compounds and methods for protection of normal cells during chemotherapy using pyrimidine-based CDK4/6 inhibitors. WO 2014/144596 filed by Strum et al. and assigned to G1 Therapeutics describes compounds and methods for protection of hematopoietic stem and progenitor cells against ionizing radiation using pyrimidine-based CDK4/6 inhibitors. WO 2014/144847 filed by Strum et al. and assigned to G1 Therapeutics describes HSPC-sparing treatments of abnormal cellular proliferation using pyrimidine-based CDK4/6 inhibitors. WO 2014/144740 filed by Strum et al. and assigned to G1 Therapeutics describes highly active anti-neoplastic and anti-proliferative pyrimidine-based CDK 4/6 inhibitors. WO 2015/161285 filed by Strum et al. and assigned to G1 Therapeutics describes tricyclic pyrimidine-based CDK inhibitors for use in radioprotection. WO 2015/161287 filed by Strum et al. and assigned to G1 Therapeutics describes tricyclic pyrimidine-based CDK inhibitors for the protection of cells during chemotherapy. WO 2015/161283 filed by Strum et al. and assigned to G1 Therapeutics describes tricyclic pyrimidine-based CDK inhibitors for use in HSPC-sparing treatments of RB-positive abnormal cellular proliferation. WO 2015/161288 filed by Strum et al. and assigned to G1 Therapeutics describes tricyclic pyrimidine-based CDK inhibitors for use as anti-neoplastic and anti-proliferative agents. WO 2016/040858 filed by Strum et al. and assigned to G1 Therapeutics describes the use of combinations of pyrimidine-based CDK4/6 inhibitors with other anti-neoplastic agents. WO 2016/040848 filed by Strum et al. and assigned to G1 Therapeutics describes compounds and methods for treating certain Rb-negative cancers with CDK4/6 inhibitors and topoisomerase inhibitors.

### Improved Patient Outcomes

The administration of a treatment described herein to the patient subgroups described herein may provide enhanced anti-tumor efficacy in patients. In some embodiments, the administration of a treatment described herein to the particular patient subgroups described above provides improved progression free survival (PFS) and/or overall survival (OS).

In certain embodiments, the treatment results in a reduction of incidents of treatment-emergent adverse events in comparison to the predicted number of incidents treatment-emergent adverse events in subjects receiving treatment without Compound I. In certain embodiments, the treatment results in a reduction of incidents of laboratory abnormalities in comparison to the predicted number of incidents of laboratory abnormalities in subjects receiving treatment without Compound I. In certain embodiments, the treatment results in an improved overall survival (OS) in comparison to the predicted overall survival (OS) in subjects receiving treatment without Compound I. In certain embodiments, the treatment results in an improved overall response rate (ORR) in comparison to the predicted overall response rate (ORR) in subjects receiving treatment without Compound I. In certain embodiments, the treatment results in an improved disease control rate (DCR) in comparison to the predicted disease control rate (DCR) in subjects receiving treatment without Compound I. In certain embodiments, the treatment results in an improved progression free survival (PFS) in comparison to the predicted progression free survival (PFS) in subjects receiving treatment without Compound I. In certain embodiments, the treatment results in an improved duration of response (DOR) in comparison to the predicted duration of response (DOR) in subjects receiving treatment without Compound I. In certain embodiments, the treatment results in an extension of time to progression (TTP) in comparison to the predicted time to progression (TTP) in subjects receiving treatment without Compound I.

### V. Standard of Care Regimens

As contemplated herein, Compound I or a pharmaceutically acceptable salt thereof or morphic form as described herein can be used in the methods described herein in conjunction with a number of standard of care chemotherapeutic treatment regimens for example, but not limited to, an ovarian cancer therapy protocol such as, but not limited to: carboplatin AUC5 IV on day 1 plus liposomal doxorubicin 30 mg/m² on day 1 every 28d for 3-6 cycles for stage I disease or 6 cycles for stage II-IV disease or high-grade stage I disease; docetaxel 60-75 mg/m² IV over 60 min followed by carboplatin AUC 5-6 IV over 30 min on day 1 every 21d for 3-6 cycles for stage I disease or 6 cycles for stage II-IV disease or high grade serous; paclitaxel 175 mg/m² over 3 hrs followed by carboplatin AUC 5-6 IV over 30 min on day 1 every 21d for 3-6 cycles; paclitaxel 80 mg/m² IV over 1h followed by carboplatin AUC 5-6 IV over 30 min on day 1, paclitaxel 80 mg/m² IV over 1h on days 8 and 15 every 21d for 6 cycles; paclitaxel 60 mg/m² over 1h followed by carboplatin AUC 2 IV over 30 min on day 1 every week for 18 weeks; paclitaxel 135 mg/m² IV over 3 hrs or IV continuous infusion over 24 hrs on day 1 and cisplatin 75-100 mg/m² intraperitoneal (IP) rapidly infused via IP port on day 2 and paclitaxel 60 mg/m² IP rapidly infused via IP port on day 8 every 21d for 6 cycles; carboplatin AUC 5 IV on day 1 and liposomal doxorubicin 30 mg/m² IV on day 1 every 28d for 6 cycles; docetaxel 60-75 mg/m² IV over 60 min followed by carboplatin AUC 5-6 IV over 30 min on day 1 every 21d for 6 cycles; paclitaxel 175 mg/m² IV over 3 hrs followed by carboplatin AUC 5-6 IV over 30 min on day 1 every 21d for 6 cycles; paclitaxel 80 mg/m² IV over 1 hr followed by carboplatin AUC 5-6 IV over 30 min on day 1 every 21d and paclitaxel 80 mg/m² IV over Ihr on days 8 and 15 for 6 cycles; paclitaxel 60 mg/m² IV over 1 hr followed by carboplatin AUC 2 IV over 30 min on day 1 every week for 18 wks; paclitaxel 175 mg/m² IV over 3 hrs followed by carboplatin AUC 5-6 IV over 30 min and bevacizumab 7.5 mg/kg IV on day 1 every 21d for 5-6 cycles and continue maintenance bevacizumab for up to 12 additional cycles; paclitaxel 175 mg/m² IV over 3 hrs followed by carboplatin AUC 6 IV over 1 hr every 21d for 6 cycles and bevacizumab 15 mg/kg IV over 30-90 min every 21d for up to 22 cycles starting day 1 of cycle 2; cisplatin 100 mg/m² IP over 90 min at time of debulking surgery; carboplatin AUC 5 IV over 30 min on day 1 every 21d for at least one cycle; paclitaxel 135 mg/m² IV and carboplatin AUC 5 IV on day 1 every 21d for 4 cycles; paclitaxel 60 mg/m² IV over 1 hr followed by carboplatin AUC 2 over 30 min on day 1 every week for 18 weeks; bevacizumab 15 mg/kg IV every 21d for at least 1 cycle; bevacizumab 7.5 mg/kg IV every 21d for at least 1 cycle; olaparib 300 mg tablet formulation twice a day every 28 day for at least 1 cycle; carboplatin AUC 5 IV on day 1 and liposomal doxorubicin 30 mg/m² IV on day 1 every 28d for 3-6 cycles; docetaxel 60-75 mg/m² IV over 60 minutes followed by carboplatin AUC 5-6 IV over 30 min on day 1 every 21d for 3-6 cycles (stage I) or 6 cycles if stage II-IV or high grade serous; paclitaxel 175 mg/m² IV over 3 hrs followed by carboplatin AUC 5-6 IV over 30 min on day 1 every 21d for 3-6 cycles; paclitaxel 80 mg/m² IV over 1 hr followed by carboplatin AUC 5-6 IV over 30 min on day 1 and paclitaxel 80 mg/m² IV over 1 hour on days 8 and 15 every 21d for 6 cycles; paclitaxel 60 mg/m² IV over 1 hr followed by carboplatin AUC 2 IV over 30 min on day 1 every week for 18 wks; paclitaxel 135 mg/m² IV over 3 hrs or IV continuous infusion over 24 hrs on day 1 and cisplatin 75-100 mg/m² IP rapidly infused via IP port on day 2 and paclitaxel 60 mg/m² IP rapidly infused via IP port every 21d for 6 cycles; carboplatin AUC 5 IV on day 1 and liposomal doxorubicin 30 mg/m² IV on day 1 every 28d for 3-6 cycles; docetaxel 60-75 mg/m² IV over 60 min followed by carboplatin AUC 5-6 IV over 30 min on day 1 every 21d for 6 cycles; paclitaxel 175 mg/m² IV over 3 hrs followed by carboplatin AUC 5-6 IV over 30 min on day 1 every 21d for 6 cycles; paclitaxel 60 mg/m² IV over 1 hr followed by carboplatin AUC 2 IV over 30 min on day 1 and paclitaxel 60 mg/m² IV over Ihr on days 8 and 15 every 21d for 6 cycles; paclitaxel 60 mg/m² IV over 1 hr followed by carboplatin AUC 2 IV over 30 min on day 1 every week for 18 wks; paclitaxel 175 mg/m² IV over 3 hrs followed by carboplatin AUC 5-6 IV over 30 min and bevacizumab 7.5 mg/kg IV on day 1 every 21d for 5-6 cycles and continue maintenance bevacizumab for up to 12 additional cycles; paclitaxel 175 mg/m² IV over 3 hrs followed by carboplatin AUC 6 IV over 1 hr on day 1 every 21d for 6 cycles and bevacizumab 15 mg/kg IV over 30-90 min on day 1 of cycle 2 every 21d for up to 22 cycles; cisplatin 100 mg/m² IP over 90 min at the time of interval debulking surgery; carboplatin AUC 5 IV and ifosfamide 3,000 mg/m² IV and mesna 1,000 mg/m² IV on day 1 every 21d for 4-6 cycles; cisplatin 20 mg/m² IV over 60 min and ifosfamide 1,500 mg/m² IV over 30 min and mesna 300 mg/m² IV over 15 min three times a day given once immediately prior to ifosfamide and 4-8 hrs after each ifosfamide dose on days 1-5 every 21d for 8 cycles if no prior radiation therapy; cisplatin 20 mg/m² over 60 min on day 1 and ifosfamide 1,200 mg/m² IV over 3 hrs and mesna 240 mg/m² IV over 15 min three times a day one dose before ifosfamide then at 4 and 8 hours from the start of each ifosfamide dose on days 1-3 every 21d for 8 cycles; gemcitabine 800-1,000 mg/m² IV over 30 min followed by carboplatin AUC 4 IV over 30 min on day 1 and gemcitabine 800-1,000 mg/m² IV over 30 min on day 8 every 21d for at least one cycle; gemcitabine 1,000 mg/m² IV over 30 min and carboplatin AUC 4 IV over 30 min and bevacizumab 15 mg/kg IV on day 1 and gemcitabine 1,000 mg/m² IV over 30 min on day 8 every 21d for 6-10 cycles followed by bevacizumab 15 mg/kg IV every 21d for at least one cycle; carboplatin AUC 5 IV over 30 min and liposomal doxorubicin 30 mg/m² IV on day 1 every 28d for at least one cycle; liposomal doxorubicin 30 mg/m² IV and carboplatin AUC 5 IV over 30 min and bevacizumab 10 mg/kg IV on day 1 and bevacizumab 10 mg/kg IV on day 15 every 28d for at least one cycle; paclitaxel 175 mg/m² IV over 3 hrs followed by carboplatin AUC 5-6 IV over 30 min on day 1 every 21d for 6 cycles; paclitaxel 60 mg/m² IV over 1 hr followed by carboplatin AUC 2 IV over 30 min on day 1 and paclitaxel 60 mg/m² IV over Ihr on days 8 and 15 every 21d for 6 cycles; paclitaxel 60 mg/m² IV over 1 hr followed by carboplatin AUC 2 IV over 30 min every week for 18 wks; paclitaxel 175 mg/m² IV over 3 hrs followed by carboplatin AUC 5-6 IV over 30 min and bevacizumab 7.5 mg/kg IV on day 1 every 21d for 5-6 cycles and continue maintenance bevacizumab up to 12 additional cycles; paclitaxel 175 mg/m² IV over 3 hrs followed by carboplatin AUC 6 IV over Ihr on day 1 ever 21d for 6 cycles and bevacizumab 15 mg/kg IV over 30-90 min on day 1 of cycle 2 every 21d for up to 22 cycles; gemcitabine 600-750 mg/m² IV over 30 min and cisplatin 30 mg/m² IV over 60 min on days 1 and 8 every 21d for at least one cycle; bevacizumab 15 mg/kg IV every 21d for at least one cycle; niraparib 300 mg orally daily every 28d for at least one cycle; Olaparib 300 mg tablet formulation twice a day every 28d for at least one cycle; rucaparib 600 mg orally twice a day every 28d for at least one cycle; albumin-bound paclitaxel 260 mg/m² IV over 30 min every 21d for at least one cycle; docetaxel 60-75 mg/m² IV over 60 min followed by carboplatin AUC 5-6 IV over 30 min on day 1 every 21d for at least one cycle; paclitaxel 80 mg/m² IV over 60 min followed by carboplatin AUC 5 IV over 30 min on day 1 and paclitaxel 80 mg/m² IV over 60 min on days 8 and 15 every 21d for 6 cycles; capecitabine 1,000 mg/m² orally twice a day on days 1-14 every 21d for at least one cycle; carboplatin AUC 5 IV over 30 min every 21d for at least one cycle; cisplatin 75 mg/m² IV over 60 min every 21d for at least one cycle; cyclophosphamide 750 mg/m² IV over 60 min every 21d for at least one cycle; doxorubicin 60 mg/m² IV push on day 1 every 21d for at least one cycle; mesna 200-240 mg/m² IV over 15 min three times a day, one dose before ifosfamide then at 4 and 8 hours from the start of each ifosfamide dose, and ifosfamide 1,000-1,200 mg/m² IV over 3 hrs on days 1-5 every 28d for at least one cycle; irinotecan 100 mg/m² IV over 30-90 min on days 1, 8, and 15 every 28d for at least one cycle; melphalan 3.5 mg/m² orally twice a day every 28d for at least one cycle; oxaliplatin 130 mg/m² IV over 2 hrs on day 1 every 21d for at least one cycle; paclitaxel 175 mg/m² IV over 3 hrs on day 1 every 21d for at least one cycle; paclitaxel 80 mg/m² (Max BSA=2 m2) IV over 60 min followed on days 1, 8, 15, and 22 every 28d for 3 cycles followed by paclitaxel 80 mg/m² (Max BSA=2 m2) over 60 min on days 1, 8, and 15 beginning with cycle 4; pemetrexed 500-900 mg/m² IV over 10 min on day 1 every 21d for at least one cycle; vinorelbine 30 mg/m² IV over 5-10 min on days 1 and 8 every 21d for at least one cycle.

In certain non-limited embodiments, Compound I or a pharmaceutically acceptable salt thereof or morphic form as described herein can be used in the methods as described herein in conjunction with a number of standard of care chemotherapeutic treatment regimens for example, but not limited to, a small cell lung cancer therapy protocol such as, but not limited to: cisplatin 60 mg/m² IV on day 1 plus etoposide 120 mg/m² IV on days 1-3 every 21d for 4 cycles; cisplatin 80 mg/m² IV on day 1 plus etoposide 100 mg/m² IV on days 1-3 every 28d for 4 cycles; cisplatin 60-80 mg/m² IV on day 1 plus etoposide 80-120 mg/m² IV on days 1-3 every 21-28d (maximum of 4 cycles); carboplatin AUC 5-6 min*mg/mL IV on day 1 plus etoposide 80-100 mg/m² IV on days 1-3 every 28d (maximum of 4 cycles); cisplatin 60-80 mg/m² IV on day 1 plus etoposide 80-120 mg/m² IV on days 1-3 every 21-28d; carboplatin AUC 5-6 min*mg/mL IV on day 1 plus etoposide 80-100 mg/m² IV on days 1-3 every 28d (maximum 6 cycles); cisplatin 60 mg/m² IV on day 1 plus irinotecan 60 mg/m² IV on days 1, 8, and 15 every 28d (maximum 6 cycles); cisplatin 30 mg/m² IV on days 1 and 8 or 80 mg/m² IV on day 1 plus irinotecan 65 mg/m² IV on days 1 and 8 every 21d (maximum 6 cycles); carboplatin AUC 5 min*mg/mL IV on day 1 plus irinotecan 50 mg/m² IV on days 1, 8, and 15 every 28d (maximum 6 cycles); carboplatin AUC 4-5 IV on day 1 plus irinotecan 150-200 mg/m² IV on day 1 every 21d (maximum 6 cycles); cyclophosphamide 800-1000 mg/m² IV on day 1 plus doxorubicin 40-50 mg/m² IV on day 1 plus vincristine 1-1.4 mg/m² IV on day 1 every 21-28d (maximum 6 cycles); Etoposide 50 mg/m² PO daily for 3wk every 4wk; topotecan 2.3 mg/m² PO on days 1-5 every 21d; topotecan 1.5 mg/m² IV on days 1-5 every 21d; carboplatin AUC 5 min*mg/mL IV on day 1 plus irinotecan 50 mg/m² IV on days 1, 8, and 15 every 28d; carboplatin AUC 4-5 IV on day 1 plus irinotecan 150-200 mg/m² IV on day 1 every 21d; cisplatin 30 mg/m² IV on days 1, 8, and 15 plus irinotecan 60 mg/m² IV on days 1, 8, and 15 every 28d; cisplatin 60 mg/m² IV on day 1 plus irinotecan 60 mg/m² IV on days 1, 8, and 15 every 28d; cisplatin 30 mg/m² IV on days 1 and 8 or 80 mg/m² IV on day 1 plus irinotecan 65 mg/m² IV on days 1 and 8 every 21d; paclitaxel 80 mg/m² IV weekly for 6wk every 8wk; paclitaxel 175 mg/m² IV on day 1 every 3wk; etoposide 50 mg/m² PO daily for 3wk every 4wk; topotecan 2.3 mg/m² PO on days 1-5 every 21d; topotecan 1.5 mg/m² IV on days 1-5 every 21d; carboplatin AUC 5 min*mg/mL IV on day 1 plus irinotecan 50 mg/m² IV on days 1, 8, and 15 every 28d; carboplatin AUC 4-5 IV on day 1 plus irinotecan 150-200 mg/m² IV on day 1 every 21d; cisplatin 30 mg/m² IV on days 1, 8, and 15 plus irinotecan 60 mg/m² IV on days 1, 8, and 15 every 28d; cisplatin 60 mg/m² IV on day 1 plus irinotecan 60 mg/m² IV on days 1, 8, and 15 every 28d; cisplatin 30 mg/m² IV on days 1 and 8 or 80 mg/m² IV on day 1 plus irinotecan 65 mg/m² IV on days 1 and 8 every 21d; paclitaxel 80 mg/m² IV weekly for 6wk every 8wk; and paclitaxel 175 mg/m² IV on day 1 every 3wk. In alternative embodiments, Compound I or a pharmaceutically acceptable salt thereof is administered to provide chemoprotection in a small cell lung cancer therapy protocol such as, but not limited to: topotecan 2.0 mg/m² PO on days 1-5 every 21d; topotecan 1.5-2.3 mg/m² PO on days 1-5 every 21d; etoposide 100 mg/m² intravenously (IV) on days 1 through 3 plus cisplatin 50 mg/m² IV on days 1 and 2 (treatment cycles administered every 3 weeks to a maximum of six cycles); etoposide 100 mg/m² intravenously (IV) on days 1 through 3 plus carboplatin 300 mg/m² IV on day 1 (treatment cycles administered every 3 weeks to a maximum of six cycles); carboplatin (300 mg/m² IV on day 1) and escalating doses of etoposide starting with 80 mg/m² IV on days 1-3; carboplatin 125 mg/m²/day combined with etoposide 200 mg/m²/day administered for 3 days; etoposide 80-200 mg/m² intravenously (IV) on days 1 through 3 plus carboplatin 125-450 mg/m² IV on day 1 (treatment cycles administered every 21-28 days); carboplatin AUC 5-6 min*mg/mL IV on day 1 plus etoposide 80-200 mg/m² IV on days 1-3 every 28d (maximum of 4 cycles).

In certain non-limited embodiments, Compound I or a pharmaceutically acceptable salt thereof or morphic form as described herein can be used in the methods described herein in conjunction with a number of standard of care chemotherapeutic treatment regimens for example, but not limited to, a CDK4/6-replication independent head and neck cancer treatment protocol, such as, but not limited to: cisplatin 100 mg/m² IV on days 1, 22, and 43 or 40-50 mg/m² IV weekly for 6-7wk; cetuximab 400 mg/m² IV loading dose 1wk before the start of radiation therapy, then 250 mg/m² weekly (pre-medicate with dexamethasone, diphenhydramine, and ranitidine); cisplatin 20 mg/m² IV on day 2 weekly for up to 7wk plus paclitaxel 30 mg/m² IV on day 1 weekly for up to 7wk; cisplatin 20 mg/m²/day IV on days 1-4 and 22-25 plus 5-FU 1000 mg/m²/day by continuous IV infusion on days 1-4 and 22-25; 5-FU 800 mg/m² by continuous IV infusion on days 1-5 given on the days of radiation plus hydroxyurea 1 g PO q12h (11 doses per cycle); chemotherapy and radiation given every other week for a total of 13wk; carboplatin 70 mg/m²/day IV on days 1-4, 22-25, and 43-46 plus 5-FU 600 mg/m²/day by continuous IV infusion on days 1-4, 22-25, and 43-46; carboplatin AUC 1.5 IV on day 1 weekly plus paclitaxel 45 mg/m² IV on day 1 weekly; cisplatin 100 mg/m² IV on days 1, 22, and 43 or 40-50 mg/m² IV weekly for 6-7wk; docetaxel 75 mg/m² IV on day 1 plus cisplatin 100 mg/m² IV on day 1 plus 5-FU 100 mg/m²/day by continuous IV infusion on days 1-4 every 3wk for 3 cycles, then 3-8wk later, carboplatin AUC 1.5 IV weekly for up to 7wk during radiation therapy; docetaxel 75 mg/m² IV on day 1 plus cisplatin 75 mg/m² IV on day 1 plus 5-FU 750 mg/m²/day by continuous IV infusion on days 1-4 every 3wk for 4 cycles; cisplatin 100 mg/m² IV on day 1 every 3wk for 6 cycles plus 5-FU 1000 mg/m²/day by continuous IV infusion on days 1-4 every 3wk for 6 cycles plus cetuximab 400 mg/m² IV loading dose on day 1, then 250 mg/m² IV weekly until disease progression (pre-medicate with dexamethasone, diphenhydramine, and ranitidine); carboplatin AUC 5 min*mg/mL IV on day 1 every 3wk for 6 cycles plus 5-FU 1000 mg/m²/day by continuous IV infusion on days 1-4 every 3wk for 6 cycles plus cetuximab 400 mg/m² IV loading dose on day 1, then 250 mg/m² IV weekly until disease progression (pre-medicate with dexamethasone, diphenhydramine, and ranitidine); cisplatin 75 mg/m² IV on day 1 plus docetaxel 75 mg/m² IV on day 1 every 3wk; cisplatin 75 mg/m² IV on day 1 plus paclitaxel 175 mg/m² IV on day 1 every 3wk; carboplatin AUC 6 IV on day 1 plus docetaxel 65 mg/m² IV on day 1 every 3wk; carboplatin AUC 6 IV on day 1 plus paclitaxel 200 mg/m² IV on day 1 every 3wk; cisplatin 75-100 mg/m² IV on day 1 every 3-4wk plus cetuximab 400 mg/m² IV loading dose on day 1, then 250 mg/m² IV weekly (pre-medicate with dexamethasone, diphenhydramine, and ranitidine); cisplatin 100 mg/m² IV on day 1 plus 5-FU 1000 mg/m²/day by continuous IV infusion on days 1-4 every 3wk; methotrexate 40 mg/m² IV weekly (3wk equals 1 cycle); paclitaxel 200 mg/m² IV every 3wk; docetaxel 75 mg/m² IV every 3wk; cetuximab 400 mg/m² IV loading dose on day 1, then 250 mg/m² IV weekly until disease progression (pre-medicate with dexamethasone, diphenhydramine, and ranitidine); cisplatin 100 mg/m² IV on day 1 every 3wk for 6 cycles plus 5-FU 1000 mg/m²/day by continuous IV infusion on days 1-4 every 3wk for 6 cycles plus cetuximab 400 mg/m² IV loading dose on day 1, then 250 mg/m² IV weekly (pre-medicate with dexamethasone, diphenhydramine, and ranitidine); carboplatin AUC 5 min*mg/mL IV on day 1 every 3wk for 6 cycles plus 5-FU 1000 mg/m²/day by continuous IV infusion on days 1-4 every 3wk for 6 cycles plus cetuximab 400 mg/m² IV loading dose on day 1, then 250 mg/m² IV weekly (pre-medicate with dexamethasone, diphenhydramine, and ranitidine); cisplatin 75 mg/m² IV on day 1 plus docetaxel 75 mg/m² IV on day 1 every 3wk; cisplatin 75 mg/m² IV on day 1 plus paclitaxel 175 mg/m² IV on day 1 every 3wk; carboplatin AUC 6 IV on day 1 plus docetaxel 65 mg/m² IV on day 1 every 3wk; carboplatin AUC 6 IV on day 1 plus paclitaxel 200 mg/m² IV on day 1 every 3wk; cisplatin 75-100 mg/m² IV on day 1 every 3-4wk plus cetuximab 400 mg/m² IV loading dose on day 1, then 250 mg/m² IV weekly (pre-medicate with dexamethasone, diphenhydramine, and ranitidine); cisplatin 100 mg/m² IV on day 1 plus 5-FU 1000 mg/m²/day by continuous IV infusion on days 1-4 every 3wk; methotrexate 40 mg/m² IV weekly (3wk equals 1 cycle); paclitaxel 200 mg/m² IV every 3wk; docetaxel 75 mg/m² IV every 3wk; cetuximab 400 mg/m² IV loading dose on day 1, then 250 mg/m² IV weekly until disease progression (pre-medicate with dexamethasone, diphenhydramine, and ranitidine); cisplatin 100 mg/m² IV on days 1, 22, and 43 with radiation, then cisplatin 80 mg/m² IV on day 1 plus 5-FU 1000 mg/m²/day by continuous IV infusion on days 1-4 every 4wk for 3 cycles; cisplatin 75 mg/m² IV on day 1 plus docetaxel 75 mg/m² IV on day 1 every 3wk; cisplatin 75 mg/m² IV on day 1 plus paclitaxel 175 mg/m² IV on day 1 every 3wk; carboplatin AUC 6 IV on day 1 plus docetaxel 65 mg/m² IV on day 1 every 3wk; carboplatin AUC 6 IV on day 1 plus paclitaxel 200 mg/m² IV on day 1 every 3wk; cisplatin 100 mg/m² IV on day 1 plus 5-FU 1000 mg/m²/day by continuous IV infusion on days 1-4 every 3wk; cisplatin 50-70 mg/m² IV on day 1 plus gemcitabine 1000 mg/m² IV on days 1, 8, and 15 every 4wk; gemcitabine 1000 mg/m² IV on days 1, 8, and 15 every 4wk or gemcitabine 1250 mg/m² IV on days 1 and 8 every 3wk; methotrexate 40 mg/m² IV weekly (3wk equals 1 cycle); paclitaxel 200 mg/m² IV every 3wk; docetaxel 75 mg/m² IV every 3wk; cisplatin 75 mg/m² IV on day 1 plus docetaxel 75 mg/m² IV on day 1 every 3wk; cisplatin 75 mg/m² IV on day 1 plus paclitaxel 175 mg/m² IV on day 1 every 3wk; carboplatin AUC 6 IV on day 1 plus docetaxel 65 mg/m² IV on day 1 every 3wk; carboplatin AUC 6 IV on day 1 plus paclitaxel 200 mg/m² IV on day 1 every 3wk; cisplatin 100 mg/m² IV on day 1 plus 5-FU 1000 mg/m²/day by continuous IV infusion on days 1-4 every 3wk; cisplatin 50-70 mg/m² IV on day 1 plus gemcitabine 1000 mg/m² IV on days 1, 8, and 15 every 4wk; gemcitabine 1000 mg/m² IV on days 1, 8, and 15 every 4wk or gemcitabine 1250 mg/m² IV on days 1 and 8 every 3wk; methotrexate 40 mg/m² IV weekly (3wk equals 1 cycle); paclitaxel 200 mg/m² IV every 3wk; and docetaxel 75 mg/m² IV every 3wk.

In certain non-limited embodiments, Compound I or a pharmaceutically acceptable salt thereof or morphic form as described herein can be used in the methods as described herein in conjunction with a number of standard of care chemotherapeutic treatment regimens for example, but not limited to, a CDK4/6-replication independent triple negative breast cancer treatment protocol such as, but not limited to: dose-dense doxorubicin (Adriamycin) and cyclophosphamide (Cytoxan) every two weeks for four cycles followed by dose-dense paclitaxel (Taxol®) every two weeks for four cycles; Adriamycin/paclitaxel/cyclophosphamide every three weeks for a total of four cycles; Adriamycin/paclitaxel/cyclophosphamide every two weeks for a total of four cycles; Adriamycin/cyclophosphamide followed by paclitaxel (Taxol®) every three weeks for four cycles each; and Adriamycin/cyclophosphamide followed by paclitaxel (Taxol®) every two weeks for four cycles each.

In certain non-limited embodiments, Compound I or a pharmaceutically acceptable salt thereof or morphic form as described herein can be used in the methods described herein in conjunction with a number of standard of care chemotherapeutic treatment regimens for example, but not limited to, a CDK4/6-replication independent bladder cancer treatment protocol such as, but not limited to: postoperative adjuvant intravesical chemotherapy for non-muscle invasive bladder cancer, first-line chemotherapy for muscle-invasive bladder cancer, and second-line chemotherapy for muscle invasive bladder cancer. Non-limiting examples of postoperative chemotherapy for bladder cancer include one dose or mitomycin (40 mg), epirubicin (80 mg), thiotepa (30 mg), or doxorubicin (50 mg). Non-limiting examples of first-line chemotherapy for bladder cancer include: gemcitabine 1000 mg/m² on days 1, 8, and 15 plus cisplatin 70 mg/m² on day 1 or 2 repeating cycle every 28 days for a total of four cycles; dosing methotrexate 30 mg/m² IV on days 1, 15, and 22 plus vinblastine 3 mg/m² IV on days 2, 15, and 22 plus doxorubicin 30 mg/m² IV on day 2 plus cisplatin 70 mg/m² IV on day 2, repeat cycle every 28d for a total of 3 cycles; and dose-dense regimens of the above administered along with doses of growth factor stimulants.

In certain non-limited embodiments, Compound I or a pharmaceutically acceptable salt thereof or morphic form as described herein can be used in the methods described herein in conjunction with a number of standard of care chemotherapeutic treatment regimens for example, but not limited to a CDK4/6-replication independent cervical cancer treatment protocol such as, but not limited to the administration of cisplatin 40 mg/m² IV once weekly, cisplatin 50-75 mg/m² IV on day 1 plus 5-fluorouracil (5-FU) 1000 mg/m² continuous IV infusion on days 2-5 and days 30-33, cisplatin 50-75 mg/m² IV on day 1 plus 5-FU 1000 mg/m² IV infusion over 24 hour on days 1-4 every 3 weeks for 3-4 cycles, bevacizumab 15 mg/kg IV over 30-90 minutes plus cisplatin on day 1 or 2 plus paclitaxel on day 1 every 3 weeks, bevacizumab plus paclitaxel on day 1 plus topotecan on days 1-3 every 3 weeks, paclitaxel followed by cisplatin on day 1 every 3 weeks, topotecan on days 1-3 followed by cisplatin on day 1 every 3 weeks, and paclitaxel on day 1 every 3 weeks. In another embodiment the cervical cancer therapy protocol is as above in addition to radiation, surgery, or another procedure.

In certain non-limited embodiments, Compound I or a pharmaceutically acceptable salt thereof or morphic form as described herein can be used in the methods described herein in conjunction with a number of standard of care chemotherapeutic treatment regimens for triple-negative breast cancer (TNBC). TNBC is defined as the absence of staining for estrogen receptor, progesterone receptor, and HER2/neu. TNBC is insensitive to some of the most effective therapies available for breast cancer treatment including HER2-directed therapy such as trastuzumab and endocrine therapies such as tamoxifen or the aromatase inhibitors. Combination cytotoxic chemotherapy administered in a dose-dense or metronomic schedule remains the standard therapy for early-stage TNBC. Platinum agents have recently emerged as drugs of interest for the treatment of TNBC with carboplatin added to paclitaxel and Adriamycin plus cyclophosphamide chemotherapy in the neoadjuvant setting. The poly (ADP-ribose) polymerase (PARP) inhibitors, including niraparib (Tesaro), are emerging as promising therapeutics for the treatment of TNBC. PARPs are a family of enzymes involved in multiple cellular processes, including DNA repair. In certain non-limited embodiments, the TNBC therapy is the PARP inhibitor niraparib. In some embodiments, Compound I or a pharmaceutically acceptable salt thereof is administered in combination with an antibody drug conjugate (ADC). In some embodiments, Compound I or a pharmaceutically acceptable salt thereof is administered in combination with an ADC selected from ado-trastuzumab emtansine (KADCYLA®), trastuzumab deruxtecan (ENHERTU®), or sacituzumab govetican (TRODELVY®).

### Embodiments

1. A method for treating a human with an abnormal cellular proliferation mediated by cyclin E1 (CCNE1) and/or cyclin E2 (CCNE2), wherein CCNE1 and/or CCNE2 are amplified or overexpressed, comprising administering an effective amount of a compound of structure:

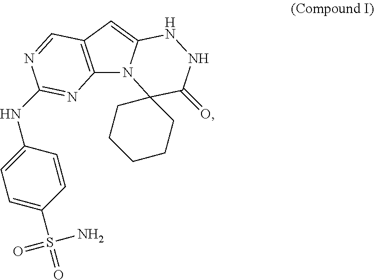 [SMILES: NS(=O)(=O)c1ccc(Nc2ncc3cc4n(c3n2)C2(CCCCC2)C(=O)NN4)cc1]

or a pharmaceutically acceptable salt thereof.
2. A method for treating a subject with an abnormal cellular proliferation, comprising:
(i) obtaining a sample from a human patient;
(ii) detecting whether cyclin E1 (CCNE1) and/or cyclin E2 (CCNE2) are over expressed in the sample compared with a control sample;
(iii) if cyclin E1 (CCNE1) and/or cyclin E2 (CCNE2) are over expressed, administering to the human patient an effective amount of a compound of structure:

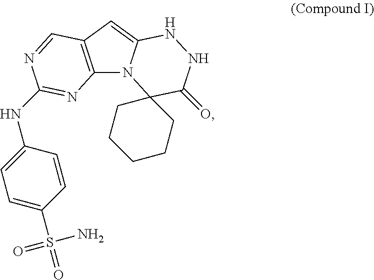 [SMILES: NS(=O)(=O)c1ccc(Nc2ncc3cc4n(c3n2)C2(CCCCC2)C(=O)NN4)cc1]

or a pharmaceutically acceptable salt thereof.
3. The method of embodiment 1 or 2, wherein the abnormal cellular proliferation is a cancer or tumor.
4. The method of any one of embodiments 1-3, wherein the abnormal cellular proliferation is selected from uterine cancer, uterine carcinosarcoma (UCS), uterine corpus endometrial carcinoma (UCEC), ovarian cancer, ovarian serous cystadenocarcinoma (OV), sarcoma (SARC), lung cancer, lung squamous cell carcinoma (LUSC), lung adenocarcinoma (LUAD), stomach cancer, stomach adenocarcinoma (STAD), bladder cancer, bladder urothelial carcinoma (BLCA), esophageal cancer, esophageal carcinoma (ESCA), adrenocortical carcinoma, breast cancer, breast invasive carcinoma (BRCA), pancreatic cancer, pancreatic adenocarcinoma (PAAD), liver cancer, liver hepatocellular carcinoma (LIHC), cervical cancer, cervical squamous cell carcinoma (CESC), endocervical adenocarcinoma, mesothelioma (MESO), head and neck squamous cell carcinoma (HSNC), colon cancer, colon adenocarcinoma (COAD), skin cancer, melanoma, skin cutaneous melanoma (SKCM), glioblastoma multiforme (GBM), kidney cancer, and kidney chromophobe (KICH).
5. The method of embodiment 3 or 4, wherein the cancer is retinoblastoma (Rb) protein-positive (Rb+).
6. The method of embodiment 3 or 4, wherein the cancer is retinoblastoma (Rb) protein-null (Rb−).
7. The method of embodiment 3, wherein the cancer is small cell lung cancer.
8. The method of embodiment 3, wherein the cancer is breast cancer.
9. The method of embodiment 8, wherein the breast cancer is hormone receptor-positive (HR+).
10. The method of embodiment 8 or 9, wherein the breast cancer is estrogen receptor-positive (ER+).
11. The method of any one of embodiments 8-10, wherein the breast cancer is progesterone receptor-positive (PR+).
12. The method of any one of embodiments 8-11, wherein the breast cancer is HER2-negative (HER2−).
13. The method of embodiment 3, wherein the cancer is ovarian cancer.
14. The method of embodiment 3, wherein the cancer is prostate cancer.
15. The method of embodiment 3, wherein the cancer is bladder cancer.
16. The method of embodiment 3, wherein the cancer is a sarcoma.
17. The method of embodiment 3, wherein the cancer is uterine cancer.
18. The method of any one of embodiments 3-17, wherein the cancer is relapsed.
19. The method of any one of embodiments 3-18, wherein the cancer has progressed following a standard of care therapy.
20. The method of any one of embodiments 3-19, wherein the cancer is intolerant or ineligible for standard or care therapy.
21. The method of any one of embodiments 3-20, wherein the cancer is refractory.
22. The method of any one of embodiments 3-21, wherein the cancer is platinum-refractory or platinum-resistant.
23. The method of any one of embodiments 3-22, wherein the cancer has progressed following a prior regimen comprising a CDK4/6 inhibitor.
24. The method of any one of embodiments 3-23, wherein the cancer is CDK4/6 inhibitor-resistant.
25. The method of any one of embodiments 3-24, wherein the cancer is intrinsically resistant to a CDK4/6 inhibitor.
26. The method of any of embodiments 3-24, wherein the cancer has acquired resistance to a CDK4/6 inhibitor.
27. The method of any one of embodiments 3-26, further comprising administering an effective amount of a CDK4/6 inhibitor.
28. The method of embodiment 27, wherein the CDK4/6 inhibitor is selected from palbociclib, ribociclib, abemaciclib, trilaciclib, lerociclib, or SHR6390 (dalpiciclib).
29. The method of embodiment 27, wherein the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.
30. The method of any one of embodiments 3-29, further comprising administering an effective amount of an anti-cancer therapy.
31. The method of embodiment 30, wherein the anti-cancer therapy is selected from radiation, surgery, immune checkpoint inhibitor, estrogen inhibitor, androgen inhibitor, PARP inhibitor, or a combination thereof.
32. The method of embodiment 29, wherein the anti-cancer therapy is an estrogen inhibitor.
33. The method of embodiment 30, wherein the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.
34. The method of embodiment 32 or 33, wherein the estrogen inhibitor is a selective estrogen receptor degrader (SERD).
35. The method of embodiment 34, wherein the SERD is selected from fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, or ZN-c5.
36. The method of embodiment 35, wherein the SERD is fulvestrant.
37. The method of embodiment 35, wherein the SERD is elacestrant (RAD1901).
38. The method of embodiment 32 or 33, wherein the estrogen inhibitor is a selective estrogen receptor modulator (SERM).
39. The method of embodiment 38, wherein the SERM is letrozole.
40. The method of any one of embodiments 1-39, further comprising administering a chemotherapeutic agent selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof.
41. The method of embodiment 40, wherein the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof.
42. The method of embodiment 41, wherein the chemotherapeutic agent is doxorubicin.
43. The method of embodiment 42, wherein the chemotherapeutic agent is camptothecin.
44. The method of embodiment 43, wherein the chemotherapeutic agent is cisplatin.
45. The method of embodiment 41, wherein the chemotherapeutic agent is carboplatin.
46. The method of embodiment 41, wherein the chemotherapeutic agent is etoposide.
47. The method of embodiments 1-46, wherein Compound I is administered in a dosage form between about 100 mg and about 800 mg.
48. The method of embodiments 1-47, wherein Compound I is administered in a dosage form selected from about 100 mg, about 200 mg, about 300 mg, about 400 mg, about 600 mg, or about 800 mg.
49. The method of embodiments 1-48, wherein Compound I is administered at least once a day.
50. The method of embodiments 1-48, wherein Compound I is administered at least twice a day.
51. The method of embodiments 1-50, wherein Compound I is administered for at least 21 days.
52. The method of embodiments 1-50, wherein Compound I is administered for at least 24 days.
53. The method of embodiments 1-50, wherein Compound I is administered for at least 28 days.
54 The method of embodiments 1-50, wherein Compound I is administered for at least 35 days.
55. The method of any one of embodiments 1-54, wherein a Next Generation Sequencing (NGS) panel test is used to confirm CCNE1 and/or CCNE2 overexpression or amplification status.
56. A method for treating a subject with a CDK4/6 inhibitor-resistant small cell lung cancer (SCLC) comprising:
(i) administering to the subject an effective amount a compound of structure:

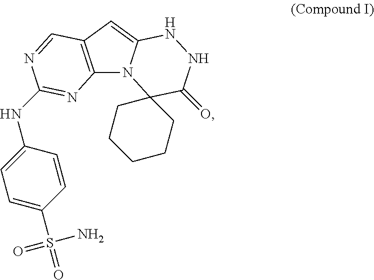 [SMILES: NS(=O)(=O)c1ccc(Nc2ncc3cc4n(c3n2)C2(CCCCC2)C(=O)NN4)cc1]

or a pharmaceutically acceptable salt thereof; and
(ii) administering to the subject an effective amount of one or more chemotherapeutic agents.
57. The method of embodiment 56, wherein the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof.
58. The method of embodiment 56 or 57, wherein the chemotherapeutic agent is doxorubicin.
59. The method of embodiment 56 or 57, wherein the chemotherapeutic agent is camptothecin.
60. The method of embodiment 56 or 57, wherein the chemotherapeutic agent is cisplatin.
61. The method of embodiment 56 or 57, wherein the chemotherapeutic agent is etoposide.
62. The method of embodiment 56 or 57, wherein the chemotherapeutic agent is carboplatin.
63. A method for treating a subject with a CDK4/6 inhibitor-resistant hormone receptor-positive (HR+) breast cancer comprising:
(i) administering to the subject an effective amount of a compound of structure

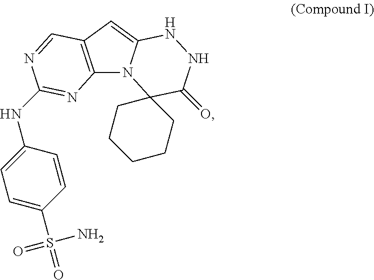 [SMILES: NS(=O)(=O)c1ccc(Nc2ncc3cc4n(c3n2)C2(CCCCC2)C(=O)NN4)cc1]

or a pharmaceutically acceptable salt thereof; and,
(ii) administering to the subject an effective amount of a CDK4/6 inhibitor.
64. The method of embodiment 63, wherein the hormone receptor-positive (HR+) breast cancer is intrinsically resistant to a CDK 4/6 inhibitor.
65. The method of embodiment 63, wherein the hormone receptor-positive (HR+) breast cancer has acquired resistance to a CDK4/6 inhibitor.
66. The method of any of embodiments 63-65, wherein the CDK4/6 inhibitor-resistant HR+ breast cancer is HER2-negative.
67. The method of any of embodiments 63 to 66, further comprising administering to the subject an effective amount of an estrogen inhibitor.
68. The method of embodiment 67, wherein the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.
69. The method of embodiment 68 wherein the estrogen inhibitor is a selective estrogen receptor modulator (SERM).
70. The method of embodiment 68, wherein the estrogen inhibitor is a selective estrogen receptor degrader (SERD).
71. The method of any one of embodiments 63-70, further comprising administering an effective amount of a chemotherapeutic agent.
72. The method of embodiment 71, wherein the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, ir irinotecan, docetaxel, temozolomide, paclitaxel, doxorubicin, camptothecin, or a combination thereof.
73. The method of any one of embodiments 63-72, wherein the CDK4/6 inhibitor is selected from palbociclib, ribociclib, abemaciclib, trilaciclib, lerociclib, SHR6390 (dalpiciclib).
74. The method of embodiment 73, wherein the CDK4/6 inhibitor is palbociclib.
75. The method of embodiment 73, wherein the CDK4/6 inhibitor is ribociclib.
76. The method of embodiment 73, wherein the CDK4/6 inhibitor is abemaciclib.
77. The method of any one of embodiments 63-72, wherein the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.
78. The method of any one of embodiments 63-77, wherein the hormone receptor-positive (HR+) breast cancer is progesterone receptor-positive (PR+).
79. The method of any one of embodiments 63-78, wherein the hormone receptor-positive (HR+) breast cancer is estrogen receptor-positive (ER+).
80. The method of any one of embodiments 63-79, wherein the hormone receptor-positive (HR+) breast cancer is HER2-negative.
81. The method of any of embodiments 63-80, wherein the hormone receptor-positive (HR+) breast cancer is resistant to an endocrine therapy.
82. The method of embodiment 81, wherein the hormone receptor-positive (HR+) breast cancer has an acquired resistance to an endocrine therapy.
83. The method of any of embodiments 81-82, wherein the hormone receptor-positive (HR+) breast cancer is resistant to a SERD.
84. The method of any of embodiments 81-83, wherein the hormone receptor-positive (HR+) breast cancer is resistant to fulverstant.
85. The method of any of embodiments 81-83, wherein the hormone receptor-positive (HR+) breast cancer is resistant to elacestrant.
86. A method for treating a subject with a CDK4/6 inhibitor-resistant estrogen receptor-positive (ER+) breast cancer comprising:
(i) administering to the subject an effective amount of a compound of structure

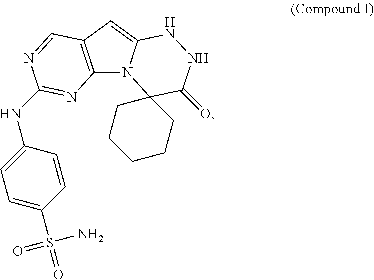 [SMILES: NS(=O)(=O)c1ccc(Nc2ncc3cc4n(c3n2)C2(CCCCC2)C(=O)NN4)cc1]

or a pharmaceutically acceptable salt thereof; and,
(ii) administering to the subject an effective amount of a CDK4/6 inhibitor.
87. The method of embodiment 86, wherein the ER+ breast cancer is intrinsically resistant to a CDK4/6 inhibitor.
88. The method of embodiment 86, wherein the ER+ breast cancer has acquired resistance to a CDK4/6 inhibitor.
89. The method of any of embodiments 86-88, wherein the CDK4/6 inhibitor-resistant ER+ breast cancer is HER2-negative.
90. The method of any of embodiments 86-89, further comprising administering an effective amount of an estrogen inhibitor.
91. The method of embodiment 90, wherein the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.
92. The method of embodiment 91, wherein the estrogen inhibitor is a selective estrogen receptor modulator (SERM).
93. The method of embodiment 91, wherein the estrogen inhibitor is a selective estrogen receptor degrader (SERD).
94. The method of any one of embodiments 86-93, further comprising administering an effective amount of a chemotherapeutic agent.
95. The method of embodiment 94, wherein the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof.
96. The method of any one of embodiments 86-95, wherein the CDK4/6 inhibitor-resistant ER+ breast cancer is luminal A breast cancer.
97. The method of any one of embodiments 86-96, wherein the CDK4/6 inhibitor is selected from palbociclib, ribociclib, abemaciclib, trilaciclib, lerociclib, SHR6390 (dalpiciclib).
98. The method of embodiment 97, wherein the CDK4/6 inhibitor is palbociclib.
99. The method of embodiment 97, wherein the CDK4/6 inhibitor is ribociclib.
100. The method of embodiment 97, wherein the CDK4/6 inhibitor is abemaciclib.
101. The method of any of embodiments 86-100, wherein the ER+ breast cancer is resistant to an endocrine therapy.
102. The method of embodiment 101, wherein the ER+ breast cancer has an acquired resistance to an endocrine therapy.
103. The method of any of embodiments 101-102, wherein the ER+ breast cancer is resistant to a SERD.
104. The method of any of embodiments 101-103, wherein the ER+ breast cancer is resistant to fulverstant.
105. The method of any of embodiments 101-103, wherein ER+ breast cancer is resistant to elacestrant.
106. The method of any one of embodiments 1-105, wherein Compound I is crystalline and characterized by an X-ray powder diffraction (XRPD) pattern comprising at least three 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
107. The method of embodiment 106, wherein the XRPD pattern comprises at least four 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
108. The method of embodiment 106, wherein the XRPD pattern comprises at least five 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
109. The method of embodiment 106, wherein the XRPD pattern comprises at least six 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
110. The method of any one of embodiments 106-109, wherein the XRPD pattern comprises at least the 2theta value of 22.4±0.2°.
111. The method of any one of embodiments 106-110, wherein the XRPD pattern comprises at least the 2theta value of 23.0±0.2°.
112. The method of any one of embodiments 106-111, wherein the XRPD pattern comprises at least the 2theta value of 17.8±0.2°.
113. A crystalline compound of structure:

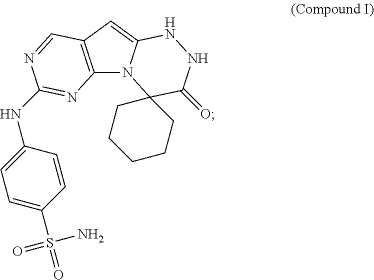 [SMILES: NS(=O)(=O)c1ccc(Nc2ncc3cc4n(c3n2)C2(CCCCC2)C(=O)NN4)cc1]

characterized by an X-ray powder diffraction (XRPD) pattern comprising at least three 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
114. The crystalline compound of embodiment 113, wherein the XRPD pattern comprises at least four 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
115. The crystalline compound of embodiment 113, wherein the XRPD pattern comprises at least five 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
116. The crystalline compound of embodiment 113, wherein the XRPD pattern comprises at least six 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
117. The crystalline compound of any one of embodiments 113-116, wherein the XRPD pattern comprises at least the 2theta value of 22.4±0.2°.
118. The crystalline compound of any one of embodiments 113-117, wherein the XRPD pattern comprises at least the 2theta value of 23.0±0.2°.
119. The crystalline compound of any one of embodiments 113-118, wherein the XRPD pattern comprises at least the 2theta value of 17.8±0.2°.
120. A crystalline compound of structure:

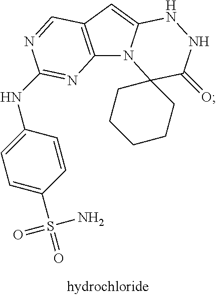 [SMILES: NS(=O)(=O)c1ccc(Nc2ncc3cc4n(c3n2)C2(CCCCC2)C(=O)NN4)cc1]

characterized by an X-ray powder diffraction (XRPD) pattern comprising at least three 2theta values selected from 7.3±0.2°, 10.7±0.2°, 15.5±0.2°, 15.6±0.2°, 16.6±0.2°, 16.9±0.2°, 19.3±0.2°, 20.0±0.2°, 24.0±0.2°, 25.1±0.2°, 25.9±0.2°, and 27.9±0.2°.
121. The crystalline compound of embodiment 120, wherein the XRPD pattern comprises at least four 2theta values selected from 7.3±0.2°, 10.7±0.2°, 15.5±0.2°, 15.6±0.2°, 16.6±0.2°, 16.9±0.2°, 19.3±0.2°, 20.0±0.2°, 24.0±0.2°, 25.1±0.2°, 25.9±0.2°, and 27.9±0.2°.
122. The crystalline compound of embodiment 120, wherein the XRPD pattern comprises at least five 2theta values selected from 7.3±0.2°, 10.7±0.2°, 15.5±0.2°, 15.6±0.2°, 16.6±0.2°, 16.9±0.2°, 19.3±0.2°, 20.0±0.2°, 24.0±0.2°, 25.1±0.2°, 25.9±0.2°, and 27.9±0.2°.
123. The crystalline compound of embodiment 120, wherein the XRPD pattern comprises at least six 2theta values selected from 7.3±0.2°, 10.7±0.2°, 15.5±0.2°, 15.6±0.2°, 16.6±0.2°, 16.9±0.2°, 19.3±0.2°, 20.0±0.2°, 24.0±0.2°, 25.1±0.2°, 25.9±0.2°, and 27.9±0.2°.
124. The crystalline compound of any one of embodiments 120-123, wherein the XRPD pattern comprises at least the 2theta value of 7.3±0.2°.
125. The crystalline compound of any one of embodiments 120-124, wherein the XRPD pattern comprises at least the 2theta value of 15.6±0.2°.
126. The crystalline compound of any one of embodiments 120-125, wherein the XRPD pattern comprises at least the 2theta value of 16.6±0.2°.
127. A pharmaceutical composition comprising a crystalline compound of any one of embodiments 113-127 and one or more pharmaceutically acceptable excipients.
128. A pharmaceutical composition comprising Compound I or a pharmaceutically acceptable salt thereof and one or more pharmaceutically acceptable excipients, wherein the pharmaceutical composition is prepared from a crystalline compound of any one of embodiments 113-127.
129. The pharmaceutical composition of embodiment 128, wherein the composition is prepared by spray drying the crystalline compound of any one of embodiments 113-127.
130. The pharmaceutical composition of embodiment 128, wherein the composition is prepared by dissolving the crystalline compound of any one of embodiments 113-127 and mixing it with a pharmaceutically acceptable excipient.
131. The pharmaceutical composition of any one of embodiments 127-130, wherein the composition comprises polyethylene glycol.
132. The pharmaceutical composition of any one of embodiments 127-131, wherein the composition comprises hydroxypropyl methylcellulose.
133. The pharmaceutical composition of any one of embodiments 127-131, wherein the composition is in a dosage form between about 100 mg to about 800 mg of Compound I, or a pharmaceutically acceptable salt thereof.
134. The pharmaceutical composition of any one of embodiments 127-132, wherein the composition is in a dosage form selected from about 100 mg, about 200 mg, about 300 mg, about 400 mg, about 600 mg, or about 800 mg of Compound I, or a pharmaceutically acceptable salt thereof.
135. A method to treat an abnormal cellular proliferation mediated by CDK2 comprising administering an effective amount of a crystalline compound of any one of embodiments 113-126 or a pharmaceutical composition of any one of embodiments 127-135 to a human in need thereof.
136. The method of embodiment 135, wherein the abnormal cellular proliferation is mediated by CDK2 is a cancer.
137. The method of embodiment 136 wherein the cancer has amplified or overexpressed cyclin E1 (CCNE1) and/or cyclin E2 (CCNE2).
138. The method of embodiments 136 or 137, wherein the cancer is retinoblastoma (Rb) protein-positive (Rb+).
139. The method of embodiments 136 or 137, wherein the cancer is retinoblastoma (Rb) protein-null (Rb−).
140. The method of any one of embodiments 136-139, wherein the cancer is advanced unresectable and/or metastatic cancer.
141. The method of any one of embodiments 136-140, wherein the cancer is platinum-refractory and/or platinum-resistant.
142. The method of any one of embodiments 136-141, wherein the cancer has progressed following a prior standard of care regimen.
143. The method of any one of embodiments 136-142, wherein the cancer has progressed following a prior standard systemic therapy.
144. The method of any one of embodiments 136-143, wherein the cancer has progressed following a prior systemic anti-cancer therapy.
145. The method of any one of embodiments 136-144, wherein the cancer has progressed following a prior regimen comprising a platinum analog.
146. The method of any one of embodiments 136-145, wherein the cancer has progressed following a prior regimen comprising a CDK4/6 inhibitor.
147. The method of any one of embodiments 135-146, wherein the abnormal cellular proliferation is selected from uterine cancer, uterine carcinosarcoma (UCS), uterine corpus endometrial carcinoma (UCEC), ovarian cancer, ovarian serous cystadenocarcinoma (OV), sarcoma (SARC), lung cancer, lung squamous cell carcinoma (LUSC), lung adenocarcinoma (LUAD), stomach cancer, stomach adenocarcinoma (STAD), bladder cancer, bladder urothelial carcinoma (BLCA), esophageal cancer, esophageal carcinoma (ESCA), adrenocortical carcinoma, breast cancer, breast invasive carcinoma (BRCA), gastric cancer, pancreatic cancer, pancreatic adenocarcinoma (PAAD), liver cancer, liver hepatocellular carcinoma (LIHC), cervical cancer, cervical squamous cell carcinoma (CESC), endocervical adenocarcinoma, mesothelioma (MESO), head and neck squamous cell carcinoma (HSNC), colon cancer, colon adenocarcinoma (COAD), skin cancer, melanoma, skin cutaneous melanoma (SKCM), glioblastoma multiforme (GBM), kidney cancer, and kidney chromophobe (KICH).
148. The method of any one of embodiments 135-146, wherein the abnormal cellular proliferation is fallopian tube cancer or primary peritoneal cancer.
149. The method of any one of embodiments 135-146, wherein the cancer is small cell lung cancer.
150. The method of any one of embodiments 135-146, wherein the cancer is breast cancer.
151. The method of embodiment 150, wherein the breast cancer is hormone receptor positive (HR+).
152. The method of embodiment 150 or 151, wherein the breast cancer is estrogen receptor positive (ER+).
153. The method of any one of embodiments 150-152, wherein the breast cancer is estrogen receptor positive (ER+).
154. The method of any one of embodiments 150-153, wherein the breast cancer is human epidermal growth factor receptor 2 negative (HER2−).
155. The method of embodiment 154, wherein the breast cancer is HR+/HER2− breast cancer.
156. The method of embodiment 154, wherein the breast cancer is ER+/HER2− breast cancer.
157. The method of any one of embodiments 135-146, wherein the cancer is ovarian cancer.
158. The method of embodiment 157, wherein the ovarian cancer has an amplification of CCNE1.
159. The method of any one of embodiments 135-146, wherein the cancer is prostate cancer.
160. The method of any one of embodiments 135-146, wherein the cancer is bladder cancer.
161. The method of any one of embodiments 135-146, wherein the cancer is a sarcoma.
162. The method of any one of embodiments 135-146, wherein the cancer is uterine cancer.
163. The method of any one of embodiments 135-162, wherein the cancer is relapsed.
164. The method of any one of embodiments 135-163, wherein the cancer is refractory.
165. The method of any one of embodiments 135-164, wherein the cancer is CDK4/6 inhibitor-resistant.
166. The method of any one of embodiments 135-165 wherein the cancer is intrinsically resistant to a CDK4/6 inhibitor.
167. The method of any one of embodiments 135-165 wherein the cancer has acquired resistance to a CDK4/6 inhibitor.
168. The method of any one of embodiments 135-167, wherein the cancer is platinum-resistant or platinum-refractory.
169. The method of any one of embodiments 135-168, further comprising administering an effective amount of a CDK4/6 inhibitor.
170. The method of embodiment 169, wherein the CDK4/6 inhibitor is selected from palbociclib, ribociclib, abemaciclib, trilaciclib, lerociclib, or SHR6390 (dalpiciclib).
171. The method of embodiment 169, wherein the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.
172. The method of any one of embodiments 135-171, further comprising administering an effective amount of an additional anti-cancer therapy.
173. The method of embodiment 172, wherein the anti-cancer therapy is selected from radiation, surgery, immune checkpoint inhibitor, estrogen inhibitor, androgen inhibitor, PARP inhibitor, or a combination thereof.
174. The method of embodiment 172, wherein the anti-cancer therapy is an estrogen inhibitor.
175. The method of embodiment 175, wherein the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.
176. The method of embodiment 175, wherein the estrogen inhibitor is a selective estrogen receptor degrader (SERD).
177. The method of embodiment 176, wherein the SERD is selected from fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, or ZN-c5.
178. The method of embodiment 177, wherein the SERD comprises fulvestrant.
179. The method of embodiment 177, wherein the SERD comprises elacestrant (RAD1901).
180. The method of embodiment 175, wherein the estrogen inhibitor is a selective estrogen receptor modulator (SERM).
181. The method of embodiment 180, wherein the SERM is letrozole.
182. The method of any one of embodiments 135-181, further comprising administering an effective amount of a chemotherapeutic agent selected from a protein synthesis inhibitor, DNA-damaging chemotherapeutic, alkylating agent, topoisomerase inhibitor, RNA synthesis inhibitor, DNA complex binder, thiolate alkylating agent, guanine alkylating agent, tubulin binder, DNA polymerase inhibitor, anticancer enzyme, RAC1 inhibitor, thymidylate synthase inhibitor, oxazophosphorine compound, integrin inhibitor, antifolate, folate antimetabolite, or a combination thereof.
183. The method of embodiment 182, wherein the chemotherapeutic agent is selected from cisplatin, carboplatin, etoposide, oxaliplatin, 5-fluorouracil, floxuridine, capecitabine, gemcitabine, mitomycin, methotrexate, vinblastine, cyclophosphamide, dacarbazine, abraxane, ifosfamide, topotecan, irinotecan, docetaxel, temozolomide, paclitaxel, doxorubicin, camptothecin, or a combination thereof.
184. The method of embodiment 183, wherein the chemotherapeutic agent is doxorubicin.
185. The method of embodiment 183, wherein the chemotherapeutic agent is camptothecin.
186. The method of embodiment 183, wherein the chemotherapeutic agent is cisplatin.
187. The method of embodiment 183, wherein the chemotherapeutic agent is carboplatin.
188. The method of embodiment 183, wherein the chemotherapeutic agent is etoposide.
189. The method of any one of embodiments 135-188, wherein the Compound I is administered in conjunction with a standard of care chemotherapeutic treatment regimen.
190. The method of any one of embodiments 135-189, wherein the crystalline compound is administered at dose of between about 100 mg and about 800 mg.
191. The method of any one of embodiments 135-189, wherein crystalline compound is administered at dose selected from about 100 mg, about 200 mg, about 300 mg, about 400 mg, about 600 mg, or about 800 mg.
192. A method for treating a human having advanced unresectable or metastatic ER+/HER2-breast cancer comprising:
(i) administering an effective amount of a CDK2 inhibitor of structure:

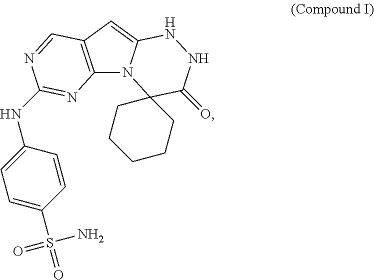 [SMILES: NS(=O)(=O)c1ccc(Nc2ncc3cc4n(c3n2)C2(CCCCC2)C(=O)NN4)cc1]

or a pharmaceutically acceptable salt thereof;
(ii) administering an effective amount of a CDK4/6 inhibitor; and,
(iii) administering an effective amount of an estrogen inhibitor, wherein the ER+/HER2− breast cancer has acquired resistance to a CDK4/6 inhibitor and a SERD.
193. The method of embodiment 192, wherein the CDK4/6 inhibitor is selected from palbociclib, ribociclib, abemaciclib, trilaciclib, lerociclib, or SHR6390 (dalpiciclib).
194. The method of embodiment 192, wherein the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.
195. The method of any one of embodiments 192-194, wherein the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.
196. The method of embodiment 195, wherein the estrogen inhibitor is a selective estrogen receptor modulator (SERM).
197. The method of embodiment 195, wherein the estrogen inhibitor is a selective estrogen receptor degrader (SERD).
198. A method for treating a human having advanced unresectable or metastatic ER+/HER2-breast cancer comprising administering an effective amount of a CDK2 inhibitor of structure:

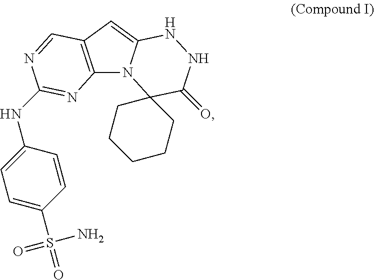 [SMILES: NS(=O)(=O)c1ccc(Nc2ncc3cc4n(c3n2)C2(CCCCC2)C(=O)NN4)cc1]

or a pharmaceutically acceptable salt thereof;
wherein Compound I is crystalline and characterized by an X-ray powder diffraction (XRPD) pattern comprising at least three 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
199. The method of embodiment 198, further comprising administering an effective amount of a CDK4/6 inhibitor.
200. The method of embodiment 198, wherein the CDK4/6 inhibitor is selected from palbociclib, ribociclib, abemaciclib, trilaciclib, lerociclib, or SHR6390 (dalpiciclib).
201. The method of embodiment 199, wherein the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.
202. The method of any one of embodiments 198-201, further comprising administering an effective amount of an estrogen inhibitor.
203. The method of embodiment 202, wherein the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.
204. The method of embodiment 203, wherein the estrogen inhibitor is a selective estrogen receptor modulator (SERM).
205. The method of embodiment 203, wherein the estrogen inhibitor is a selective estrogen receptor degrader (SERD).
206. The method of any one of embodiments 192-205, wherein the human previously received at least one prior line of endocrine therapy.
207. The method of any one of embodiments 192-206, wherein the human previously received at least one prior line of CDK4/6 inhibitor therapy.
208. The method of any one of embodiments 192-207, wherein the human previously received at least one prior line of chemotherapy.
209. The method of any one of embodiments 192-208, wherein the human previously received at least two prior lines of chemotherapy.
210. The method of any one of embodiments 192-209, wherein the advanced unresectable or metastatic ER+/HER2− breast cancer has progressed following a prior regimen comprising a CDK4/6 inhibitor.
211. The method of any one of embodiments 191-210, wherein Compound I is administered in conjunction with a standard of care chemotherapeutic treatment regimen.
212. A method for treating a human having advanced or metastatic epithelial ovarian cancer with amplification of CCNE1 comprising administering an effective amount of a CDK2 inhibitor of structure:

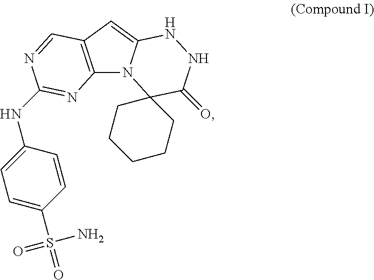 [SMILES: NS(=O)(=O)c1ccc(Nc2ncc3cc4n(c3n2)C2(CCCCC2)C(=O)NN4)cc1]

or a pharmaceutically acceptable salt thereof;
wherein Compound I is crystalline and characterized by an X-ray powder diffraction (XRPD) pattern comprising at least three 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
213. The method of embodiment 212, wherein the advanced or metastatic epithelial ovarian cancer with amplification of CCNE1 is platinum-resistant or platinum-refractory.
214. The method of embodiment 212 or 213, wherein the advanced or metastatic epithelial ovarian cancer comprises fallopian tube cancer.
215. The method of embodiment 212 or 213, wherein the advanced or metastatic epithelial ovarian cancer comprises primary peritoneal cancer.
216. A method for treating a human having advanced or metastatic fallopian tube cancer with amplification of CCNE1 comprising administering an effective amount of a CDK2 inhibitor of structure:

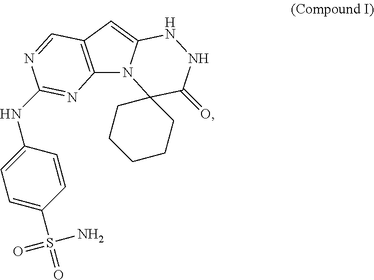 [SMILES: NS(=O)(=O)c1ccc(Nc2ncc3cc4n(c3n2)C2(CCCCC2)C(=O)NN4)cc1]

or a pharmaceutically acceptable salt thereof;
   wherein Compound I is crystalline and characterized by an X-ray powder diffraction (XRPD) pattern comprising at least three 2theta values selected from 10.3±0.2°, 11.90.2°, 16.3±0.2°, 17.8±0.2°, 19.3±0.2°, 22.4±0.2°, 23.0±0.2°, 24.1±0.2°, 24.7±0.2°, and 30.0±0.2.
217. The method of embodiment 216, wherein the advanced or metastatic fallopian tube cancer with amplification of CCNE1 is platinum-resistant or platinum-refractory.
218. The method of embodiment 216 or 217, further comprising administering an effective amount of one or more additional bioactive agents.
219. A method for treating a human having advanced or metastatic primary peritoneal cancer with amplification of CCNE1 comprising administering an effective amount of a CDK2 inhibitor of structure:

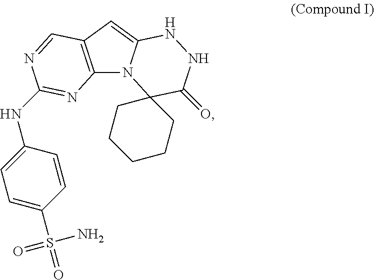 [SMILES: NS(=O)(=O)c1ccc(Nc2ncc3cc4n(c3n2)C2(CCCCC2)C(=O)NN4)cc1]

or a pharmaceutically acceptable salt thereof;
   wherein Compound I is crystalline and characterized by an X-ray powder diffraction (XRPD) pattern comprising at least three 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
220. The method of embodiment 219, wherein the advanced or metastatic primary peritoneal cancer with amplification of CCNE1 is platinum-resistant or platinum-refractory.
221. The method of embodiment 219 or 220, further comprising administering an effective amount of one or more additional bioactive agents.
222. The method of any one of embodiments 212-221, wherein the cancer has progressed following a prior standard of care therapy.
223. The method of any one of embodiments 212-222, wherein the cancer has progressed following a prior anti-cancer therapy.
224. The method of any one of embodiments 212-223, wherein the cancer has progressed following a prior regimen comprising a platinum analog.
225. The method of any one of embodiments 212-224, wherein Compound I is administered in conjunction with a standard of care chemotherapeutic treatment regimen.
226. A method for treating a human having an advanced or metastatic solid tumor comprising administering an effective amount of a CDK2 inhibitor of structure:

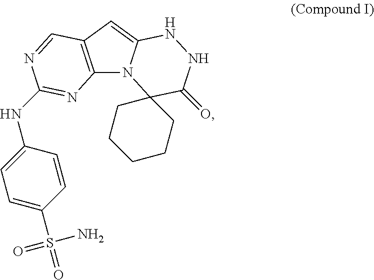 [SMILES: NS(=O)(=O)c1ccc(Nc2ncc3cc4n(c3n2)C2(CCCCC2)C(=O)NN4)cc1]

or a pharmaceutically acceptable salt thereof;
   wherein Compound I is crystalline and characterized by an X-ray powder diffraction (XRPD) pattern comprising at least three 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.0±0.2.
227. The method of embodiment 226, wherein the advanced or metastatic solid tumor has progressed following a prior standard of care regimen.
228. The method of embodiment 226 or 227, wherein the advanced or metastatic solid tumor is intolerant to or is ineligible for standard therapy.
229. The method of any one of embodiments 226-228, wherein the advanced or metastatic solid tumor comprises an amplification of CCNE1.
230. The method of any one of embodiments 226-229, further comprising administering an effective amount of a CDK4/6 inhibitor.
231. The method of embodiment 230 wherein the CDK4/6 inhibitor is selected from palbociclib, ribociclib, abemaciclib, trilaciclib, lerociclib, or SHR6390 (dalpiciclib).
232. The method of embodiment 230, wherein the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.
233. The method of any one of embodiments 226-232, further comprising administering an effective amount of an anti-cancer therapy.
234. The method of embodiment 233, wherein the anti-cancer therapy is selected from chemotherapeutic agent, radiation, surgery, immune checkpoint inhibitor, estrogen inhibitor, androgen inhibitor, PARP inhibitor, or a combination thereof.
235. The method of embodiment 234, wherein the anti-cancer therapy is an estrogen inhibitor.
236. The method of embodiment 235, wherein the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.
237. The method of embodiment 236, wherein the estrogen inhibitor is a selective estrogen receptor degrader (SERD).
238. The method of embodiment 237, wherein the SERD is selected from fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, or ZN-c5.
239. The method of embodiment 238, wherein the SERD comprises fulvestrant.
240. The method of embodiment 238, wherein the SERD comprises elacestrant (RAD1901).
241. The method of embodiment 236, wherein the estrogen inhibitor is a selective estrogen receptor modulator (SERM).
242. The method of embodiment 241, wherein the SERM comprises letrozole.
243. The method of any one of embodiments 226-242, wherein Compound I is administered in conjunction with a standard of care chemotherapeutic treatment regimen.
244. A method for treating a human having hormone receptor-positive (HR+) advanced breast cancer comprising:
(i) administering an effective amount of a CDK2 inhibitor of structure:

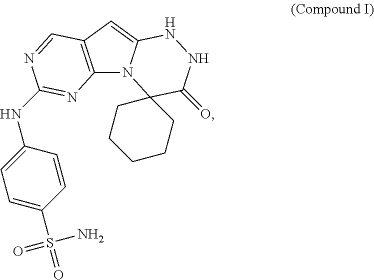 [SMILES: NS(=O)(=O)c1ccc(Nc2ncc3cc4n(c3n2)C2(CCCCC2)C(=O)NN4)cc1]

or a pharmaceutically acceptable salt thereof; and
(ii) administering an effective amount of fulvestrant;
   wherein Compound I is crystalline and characterized by an X-ray powder diffraction (XRPD) pattern comprising at least three 2theta values selected from 10.3±0.2°, 11.9±0.2°, 16.3±0.2°, 17.8±0.2°, 19.3±0.2°, 22.4±0.2°, 23.0±0.2°, 24.1±0.2°, 24.7±0.2°, and 30.00.2.
245. The method of embodiment 244, further comprising administering an effective amount of a CDK4/6 inhibitor.
246. The method of embodiment 245, wherein the CDK4/6 inhibitor is selected from palbociclib, ribociclib, abemaciclib, trilaciclib, lerociclib, or SHR6390 (dalpiciclib).
247. The method of embodiment 245, wherein the CDK4/6 inhibitor is selected from BPI-16350, narazaciclib (ON-123300), FLX-925 (AMG-925), UCT-03-008, GLR2007, birociclib
(XZP-3287), LY5219, PF-07220060, or ON-123300.
248. A method for treating a human having hormone receptor-positive (HR+) advanced breast cancer comprising:
(i) administering an effective amount of a CDK2 inhibitor of structure:

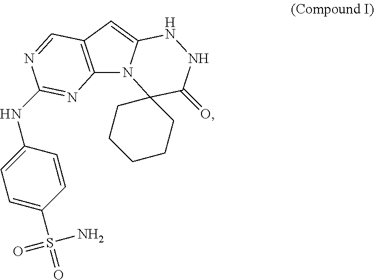 [SMILES: NS(=O)(=O)c1ccc(Nc2ncc3cc4n(c3n2)C2(CCCCC2)C(=O)NN4)cc1]

or a pharmaceutically acceptable salt thereof; and
(ii) administering an effective amount of fulvestrant; and,
(iii) administering an effective amount of a CDK4/6 inhibitor.
249. The method of embodiment 248, wherein the CDK4/6 inhibitor is selected from palbociclib, ribociclib, abemaciclib, trilaciclib, lerociclib, or SHR6390 (dalpiciclib).
250. The method of embodiment 249, wherein the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.
251. The method of any one of embodiments 244-250, wherein the HR+ advanced breast cancer is HER2−.
252. The method of any one of embodiments 244-251, wherein the method is administered as a first-line (1L) therapy.
253. The method of any one of embodiments 244-251, wherein the human previously received at least one prior line of endocrine therapy.
254. The method of any one of embodiments 244-251 or 253, wherein the HR+ advanced breast cancer has progressed following a prior standard of care regimen.
255. A method for treating a human having CDK4/6 inhibitor resistant and endocrine therapy-resistant cancer comprising:
(i) administering an effective amount of a CDK2 inhibitor of structure:

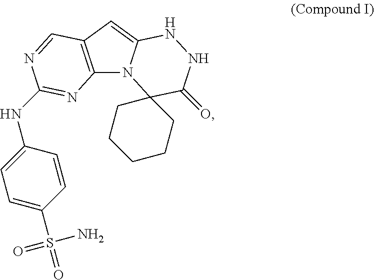 [SMILES: NS(=O)(=O)c1ccc(Nc2ncc3cc4n(c3n2)C2(CCCCC2)C(=O)NN4)cc1]

or a pharmaceutically acceptable salt thereof; and
(ii) administering an effective amount of an estrogen inhibitor.
256. The method of embodiment 255, wherein the estrogen inhibitor is selected from a selective estrogen receptor modulator (SERM), selective estrogen receptor degrader (SERD), complete estrogen receptor degrader, complete estrogen antagonist, partial estrogen antagonist, or a combination thereof.
257. The method of embodiment 256, wherein the estrogen inhibitor is a selective estrogen receptor degrader (SERD).
258. The method of embodiment 256, wherein the SERD is selected from fulvestrant, rintodestrant (G1T48), borestrant (ZB-716), brilanestrant (GDC0810), camizestrant (AZD9833), D00502, elacestrant (RAD1901), etacstil (GW5638), GW7604, AZD9496, GDC-0927, giredestrant (GDC9545, RG6171), LSZ102, imlunestrant (LY3484356), SAR439859, SCR6852, or ZN-c5.
259. The method of embodiment 258, wherein the SERD comprises fulvestrant.
260. The method of embodiment 258, wherein the SERD comprises elacestrant (RAD1901).
261. The method of embodiment 256, wherein the estrogen inhibitor is a selective estrogen receptor modulator (SERM).
262. The method of embodiment 261, wherein the SERM comprises letrozole.
263. The method of any one of embodiments 255-262, further comprising administering an effective amount of a CDK4/6 inhibitor.
264. The method of embodiment 263, wherein the CDK4/6 inhibitor is selected from palbociclib, ribociclib, abemaciclib, trilaciclib, lerociclib, or SHR6390 (dalpiciclib).
265. The method of embodiment 263, wherein the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300.
266. The method of any one of embodiments 255-265, wherein the cancer is selected from breast cancer, ovarian cancer, endometrial cancer, prostate cancer, or uterine cancer.
267. The method of embodiment 266, wherein the cancer is breast cancer.
268. The method of embodiment 267, wherein the breast cancer is hormone receptor positive (HR+).
269. The method of any of embodiments 267 or 268, wherein the breast cancer is estrogen receptor positive (ER+).
270. The method of any one of embodiments 266-269, wherein the breast cancer is progesterone receptor-positive (PR+).
271. The method of any one of embodiments 266-270, wherein the breast cancer is human epidermal growth factor receptor 2 negative (HER2−).
272. The method of embodiment 271, wherein the breast cancer is ER+/HER2− breast cancer.
273. The method of embodiment 271, wherein the breast cancer is HR+/HER2− breast cancer.
274. The method of embodiment 267, wherein the breast cancer is luminal A breast cancer.
275. The method of any one of embodiments 255-274, wherein the human previously received at least one prior line of CDK4/6 inhibitor therapy.
276. The method of embodiment any one of embodiments 255-275, wherein the human previously received at least one prior line of endocrine therapy.
277. The method of any one of embodiments 255-276, wherein the cancer has progressed following a prior standard systemic therapy.
278. The method of any one of embodiments 255-277, wherein the cancer has progressed following a prior regimen comprising a CDK4/6 inhibitor.
279. The method of any one of embodiments 255-278, wherein Compound I is administered in conjunction with a standard of care chemotherapeutic treatment regimen.
280. The method of any one of embodiments 135-279, wherein Compound I is administered at least once a day.
281. The method of any one of embodiments 135-279, wherein Compound I is administered at least twice a day.
282. The method of embodiment 280 or 281, wherein Compound I is administered for at least 21 days, at least 24 days, at least 28 days, or longer than 28 days.
283. The method of embodiment 282, wherein Compound I is administered at least once a day for at least 28 days.
284. The method of any one of embodiments 1-112 or 135-283, wherein the treatment results in a reduction of incidents of treatment-emergent adverse events in comparison to the predicted number of incidents of treatment-emergent adverse events in subjects receiving treatment without Compound I.
285. The method of any one of embodiments 1-112 or 135-284, wherein the treatment results in a reduction of incidents of laboratory abnormalities in comparison to the predicted number of incidents of laboratory abnormalities in subjects receiving treatment without Compound I.
286. The method of any one of embodiments 1-112 or 135-285, wherein the treatment results in an improved overall survival (OS) in comparison to the predicted overall survival (OS) in subjects receiving treatment without Compound I.
287. The method of any one of embodiments 1-112 or 135-286, wherein the treatment results in an improved overall response rate (ORR) in comparison to the predicted overall response rate (ORR) in subjects receiving treatment without Compound I.
288. The method of any one of embodiments 1-112 or 135-287, wherein the treatment results in an improved disease control rate (DCR) in comparison to the predicted disease control rate (DCR) in subjects receiving treatment without Compound I.
289. The method of any one of embodiments 1-112 or 135-288, wherein the treatment results in an improved progression free survival (PFS) in comparison to the predicted progression free survival (PFS) in subjects receiving treatment without Compound I.
290. The method of any one of embodiments 1-112 or 135-289, wherein the treatment results in an improved duration of response (DOR) in comparison to the predicted duration of response (DOR) in subjects receiving treatment without Compound I.
291. The method of any one of embodiments 1-112 or 135-290, wherein the treatment results in an extension of time to progression (TTP) in comparison to the predicted time to progression (TTP) in subjects receiving treatment without Compound I.

### VI. Examples

### Example 1. CDK Inhibition In Vitro Assays

Selected CDK binding partner compounds disclosed herein were tested in kinase assays by Nanosyn (Santa Clara, CA) to determine their inhibitory effect on respective CDK partners. Kinase phosphorylation assays were performed using a caliper technology microfluidics platform (Caliper Assay Platform). With this system, the target enzyme is incubated with fluorescently labeled substrate and test compounds in a standardized reaction mixture in 384 well plates. Upon termination of the reaction, samples are introduced onto microfluidic chips. The samples migrate through channels in the chips and product and substrate are separated based on the difference in their charge and mass (electrophoretic mobility shift). Enzyme activity is measured by direct comparison of the fluorescence in the product and substrate peaks.

Compound I Preferentially Complexes with and Inhibits CDK2

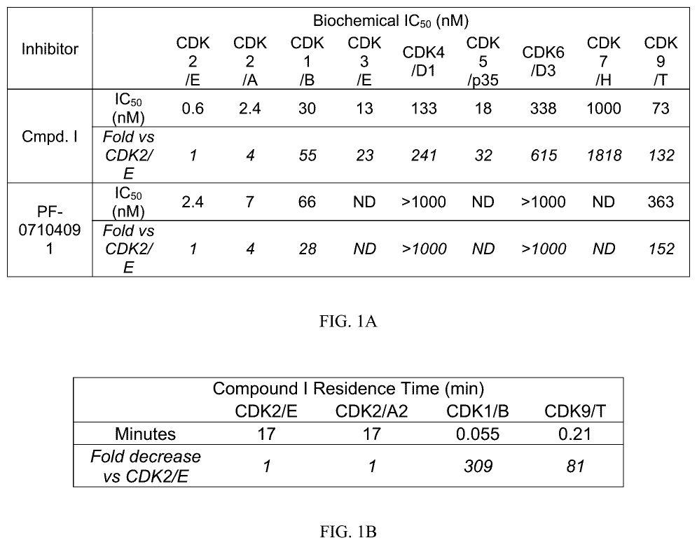

Protein kinase activity was measured against multiple CDKs and their binding partners: CDK1/Cyclin B (CDK1/B), CDK2/Cyclin A (CDK2/A), and CDK2/Cyclin E (CDK2/E), CDK3/Cyclin E (CDK3/E), CDK4/Cyclin D1 (CDK4/D1), CDK6/Cyclin D3 (CDK6/D3), CDK5/p35 (CDK5/p35), CDK7/Cyclin H (CDK7/H), CDK9/Cyclin T1 (CDK9/T). Test compounds were diluted in 100% DMSO using 3-fold dilution steps. Final compound concentration in the assay ranged from 1 μM to 0.0056 nM. Compounds were tested in a single well for each dilution, and the final concentration of DMSO in all assays was kept at 1%. The Pfizer selective CDK2 inhibitor compound, PF-07104091, was included as a reference compound. Compound I provided the greatest inhibition of CDK2/E and CDK2/A when compared with other CDK/cyclin combinations (FIG. 1A). Compound I exhibited 4-fold greater inhibition (0.6 nM) of CDK2/E than the Pfizer selective CDK2 inhibitor compound PF-07104091 (2.4 nM).

A TR-FRET binding assay was used to determine the kinetic constants (kon and koff), Kd and residence times of compounds upon binding to the target of interest. Each compound was tested using 4-point (1, 10, 100 and 1000 nM), 10-fold dilution series in singlicate. Wells containing a fluorescent probe, the antibody and the target (CDK1/B1, CDK2/A2. CDK2/E1, CDK9/T) without compound were used as the total binding control and wells containing the fluorescent probe and the antibodies were used as the basal control. The TR-FRET signal was measured continuously throughout 240 minutes. Results were fitted into a non-linear regression curve that measures the kinetics of competitive binding.

Compound I had a substantially long residence times with CDK2/E (17 min) and CDK2/A (17 min) than other CDK/cyclin combinations (FIG. 1B). Residence time is the lifetime of compound-target interaction. For example, Compound I resided 309-fold and 81-fold less time with CDK1/B and CDK9/T, respectively, compared to CDK2/E and/or CDK2/A. These results demonstrate Compound I stably complexes with CDK2/E and CDK/A and potentially regulates long-lasting inhibition of CDK2.

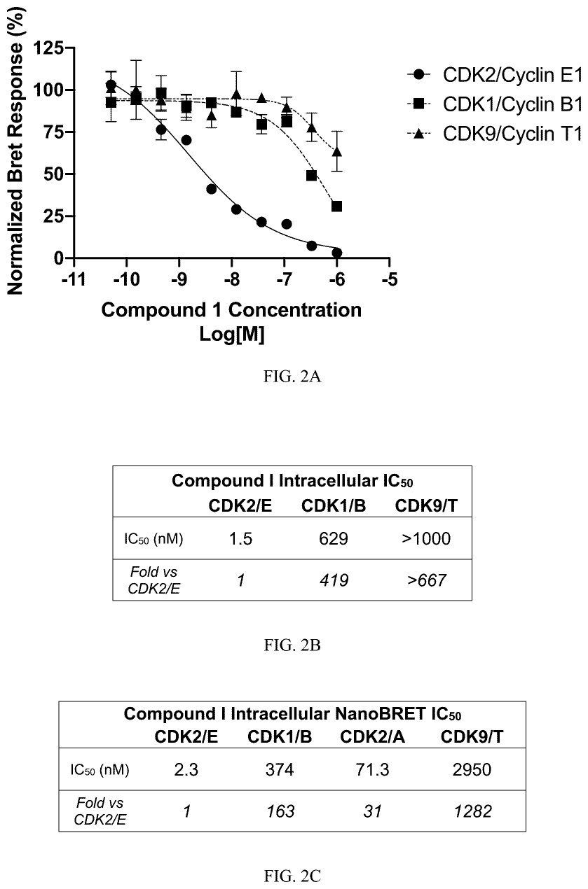

Compound I was also tested in the NanoBRET™ Target Engagement Intracellular Kinase Assay (FIG. 2A-B). HEK293 cells were transfected with CDK1+Cyclin B1, CDK2+Cyclin E1 and CDK9+Cyclin T1 (1 μg+9 μg)-NanoLuc Fusion Vectors. The transfected cells were treated with compound 1 (starting at 1 μM, 10-dose with 3-fold dilution) for 1 hour. CDK2+Cyclin E1 target engagement was measured by NanoBRET™ assay. Curve fits were performed only when % NanoBRET™ signal at the highest concentration of compounds was less than 55%. As the concentration of Compound I increased, the acceptor: donor emission ratio (the BRET ratio) of CDK2/E decreased (FIG. 2A).

FIG. 2B is a table that represents the IC₅₀ determination of Compound I against CDK2/E, CDK1/B, CDK9/T as measured using NanoBRET technology. In addition, CDK1/B and CDK9/T values were compared to CDK2/E. The IC₅₀ values for Compound I for binding to CDK2/E, CDK1/B, and CDK9/T indicate Compound I exhibited the most potent binding of CDK2/E in cells (FIG. 2B), over 400-fold greater than that of CDK1/B or CDK9/T.

FIG. 2C is a table that represents an independent study assessing IC₅₀ values of Compound I against CDK2/E, CDK1/B, CDK2/A, CDK4/D1, CDK6/D3, and CDK9/T as measured using NanoBRET technology. In addition, CDK1/B, CDK2/A, and CDK9/T values were compared to CDK2/E. The IC₅₀ values for Compound I for binding to CDK2/E, CDK1/B, CDK2/A, and CDK9/T indicate Compound I exhibited the most potent binding of CDK2/E in cells (FIG. 2C), over 30-fold greater than CDK2/A and over 150-fold and 1200-fold greater than that of CDK1/B or CDK9/T, respectively.

These results indicate that Compound I is a potent and selective CDK2 inhibitor.

### Example 2. Compound I Exhibits Favorable Safety Characteristics in Non-Cancerous Human Cells

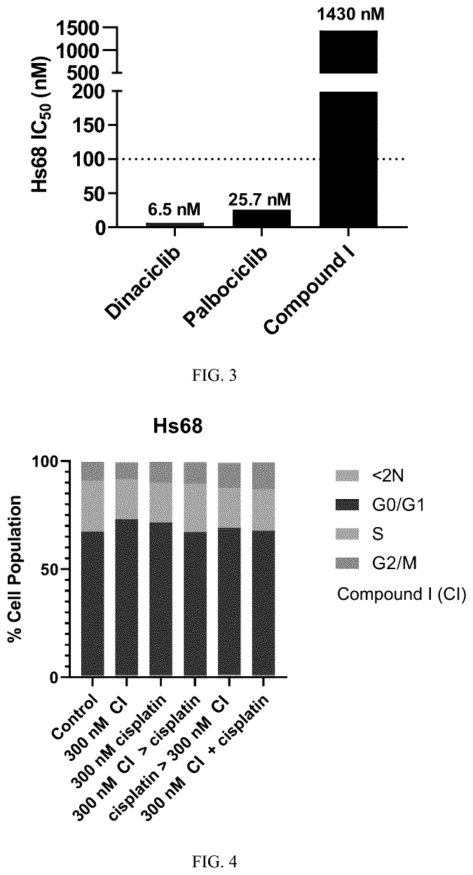

Hs68 foreskin fibroblast cells were used to model normal, healthy (non-cancerous cells). The Hs68 Normal foreskin fibroblast cell line was grown in DMEM-high glucose/1× Glutamax/10% fetal bovine serum and was plated at 1,000 cells/well in Corning 96 well white wall/clear bottom tissue culture treated plates. Cells were incubated at 37° C. for 24 hours prior to addition of dinaciclib (positive control; CDK1/5/9 inhibitor), Compound I or PF-07104091 (selective CDK2 inhibitor) from 1 nM to 10 μM as 9-point dose response in triplicate for 6 days. Plates were removed from incubator and treated with CellTiter-Glo (Promega, Inc) following manufacturer's specifications. Plates were then read on Biotek luminometer with 1 sec/well integration. Results were converted to Microsoft Excel and transferred to Graphpad Prism for analysis and IC₅₀ determination. Compound I is substantially less potent in the Hs68 normal foreskin fibroblast cell line compared with dinaciclib and palbociclib (FIG. 3).

Compound I Induces Few Cell Cycle Changes in Normal Human Fibroblast Hs68 Cells with Combinations of Compound I and Cisplatin Combinations

The Hs68 normal foreskin fibroblast cell line was grown in DMEM-high glucose/1× Glutamax/10% fetal bovine serum and was plated at 30,000 cells/well in Corning 6 well tissue culture treated plates. Cells were incubated at 37° C. for 24 hours prior to addition of the first treatment. Initial treatments were 0.1% DMSO (control), 300 nM Compound I, 300 nM cisplatin, or a combination of 300 nM Compound I and 300 nM cisplatin. After 6 hours, one well containing Compound I, had cisplatin added to 300 nM, and one well containing cisplatin had Compound I added to 300 nM. Cells were treated to 24 hours post-initial treatment, with 10 μM EdU added at hour 23. Cells were collected and fixed in 1% paraformaldehyde. Cells were incubated with pacific blue azide dye as described by Thermo Scientific. Cells were then incubated with antibody against γ-H2AX at a ratio of 1:50 before incubation with FarRed dye and RNase. Cells were analyzed on a BD Celesta and data compiled using FloJo and GraphPad. Cell cycle profiles were evaluated following treatment via Flow Cytometry using FlowJo (v10.0) software. Few cell cycle changes were observed in the Hs68 normal foreskin fibroblast cell line following 24 hours of incubation with Compound I either alone or in combination with cisplatin (FIG. 4).

Compound I does not Induce Caspase Activation in Normal Human Fibroblast Hs68 Cells

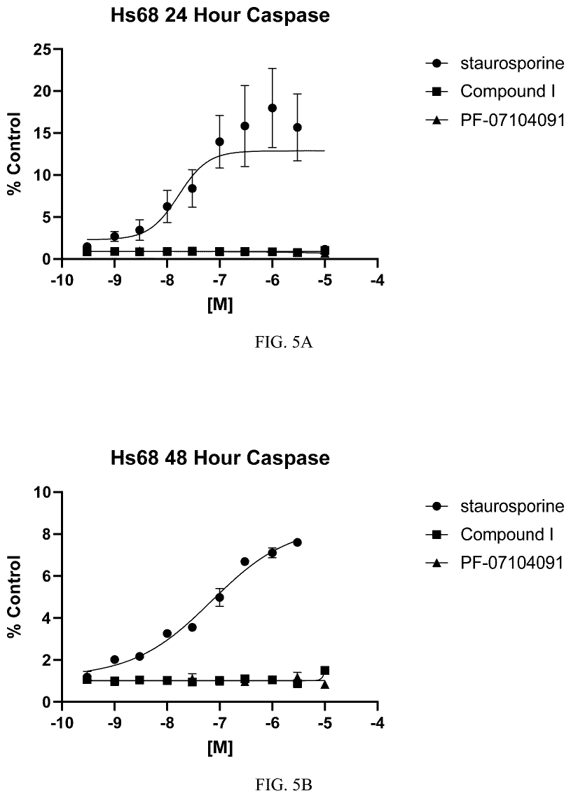

The Hs68 Normal foreskin fibroblast cell line was grown in DMEM-high glucose/1× Glutamax/10% fetal bovine serum and was plated at 1,000 cells/well in Corning 96 well white wall/clear bottom tissue culture treated plates. Cells were incubated at 37° C. for 24 hours prior to addition of staurosporine (positive control), Compound I or PF-07104091 from 300 pM to 10 μM in triplicate for 24, 48 or 72 hours. Plates were removed from incubator at each specified timepoint and treated with CaspaseGlo-3/7 (Promega, Inc) following manufacturer specifications. Plates were then read on Biotech luminometer with 1 sec/well integration. Results were converted to Microsoft Excel and transferred to GraphPad Prism for Analysis (FIG. 5A-C). Staurosporine was the only treatment condition which induced a substantial increase in the percent of cells that were positive for caspase 3/7, a marker of cell death.

Compound I does not Induce Increased yH2AX Levels in Normal Human Fibroblast Hs68 Cells

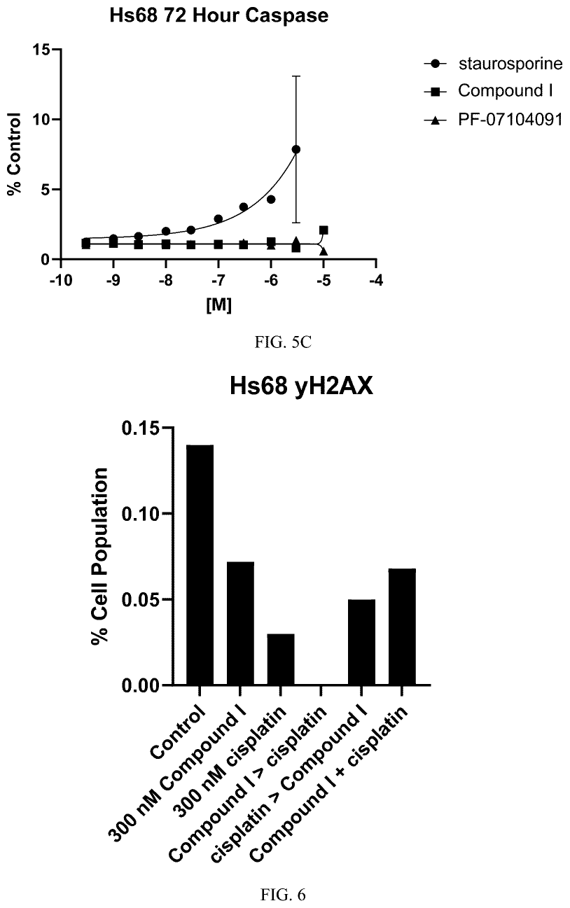

The Hs68 Normal foreskin fibroblast cell line was grown in DMEM-high glucose/1× Glutamax/10% fetal bovine serum and was plated at 30,000 cells/well in Corning 6 well tissue culture treated plates. Cells were incubated at 37° C. for 24 hours prior to addition of the first treatment. Initial treatments were 0.1% DMSO control, 300 nM Compound I, 300 nM cisplatin, or a combination of 300 nM Compound I and 300 nM cisplatin. After 6 hours, one well containing Compound I, had cisplatin added to 300 nM, and one well containing cisplatin had Compound I added to 300 nM. Cells were treated to 24 hours post-initial treatment, with 10 μM EdU added at hour 23. Cells were collected and fixed in 1% paraformaldehyde. Cells were incubated with pacific blue azide dye as described by Thermo Scientific. Cells were then incubated with antibody against γ-H2AX at a ratio of 1:50 before incubation with FarRed dye and RNase. Cells were analyzed on a BD Celesta and data compiled using FloJo and GraphPad (FIG. 6). Compound I either alone or in combination with cisplatin induced a lower percent of γ-H2AX-positive cells compared with control.

### Example 3. Compound I Potently Inhibits CDK4/6 Inhibitor-Resistant Cancers

Treatment regimens involving the administration of CDK4/6 inhibitors, such as palbociclib, are a core arm of current standard of care treatments for breast cancers (O'Leary et al. Nat Rev Clin Oncol. 13(7): 417-30(2016)). Unfortunately, most breast tumors that initially respond to CDK4/6 inhibition eventually develop resistance over time (Ono et al. J Cancer Res Clin Oncol. 147(11): 3211-24(2021)). Novel therapeutics that can overcome resistance to current treatment strategies are highly desirable.

### Enhanced Inhibition of Palbociclib-Resistant Breast Cancer Cells by the Combined Administration of Compound I and Palbociclib

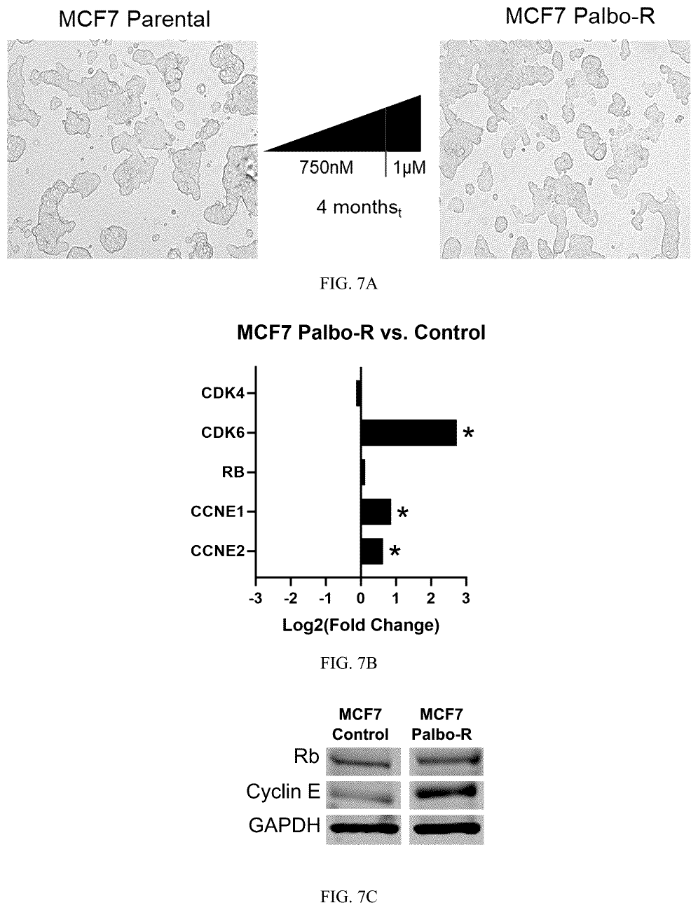

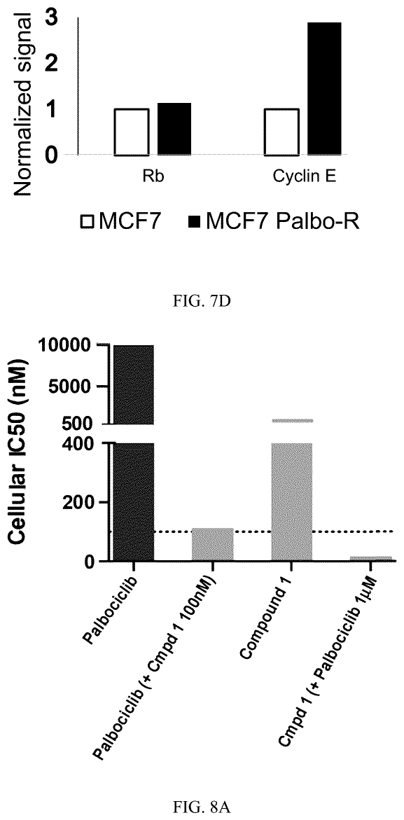

MCF7 Parental cells (ATCC, ER+ Her2− BC, CDK4/6 dependent) were maintained in culture for four months in complete media (EMEM/10% FBS/glutamax/insulin) as a control. MCF7 palbociclib-resistant (Palbo-R) cells were generated by maintaining MCF7 cells in complete media plus palbociclib for three months at ˜IC₉₀ (750 nM) followed by one month at 1 uM. Pictures of the cells in culture demonstrate similar morphology and growth patterns of the parental and Palbo-R cell lines (FIG. 7A). Whole transcriptome profiling was performed on MCF7 parental cells and Palbo-R MCF7 cells by RNA-Seq. Libraries were prepared using the Illumina TruSeq Stranded mRNA assay and paired-end sequenced (2×50 bp) on the Illumina HiSeq platform. The fold change (log 2-transformed) of the transcript levels of specific genes in MCF7 Palbo-R vs. MCF7 parental demonstrate key expression changes as a result of the adaptation of palbociclib resistance (FIG. 7B). The Western blot analysis of MCF7 parental and MCF7 palbo-R cells demonstrated an increase in the ratio of cyclin E to Rb levels in Palbo-R cells compared to the MCF7 parental cells, with quantitation of normalized signal to loading control, GAPDH, located at the bottom of the Western blot figure (FIG. 7C-D).

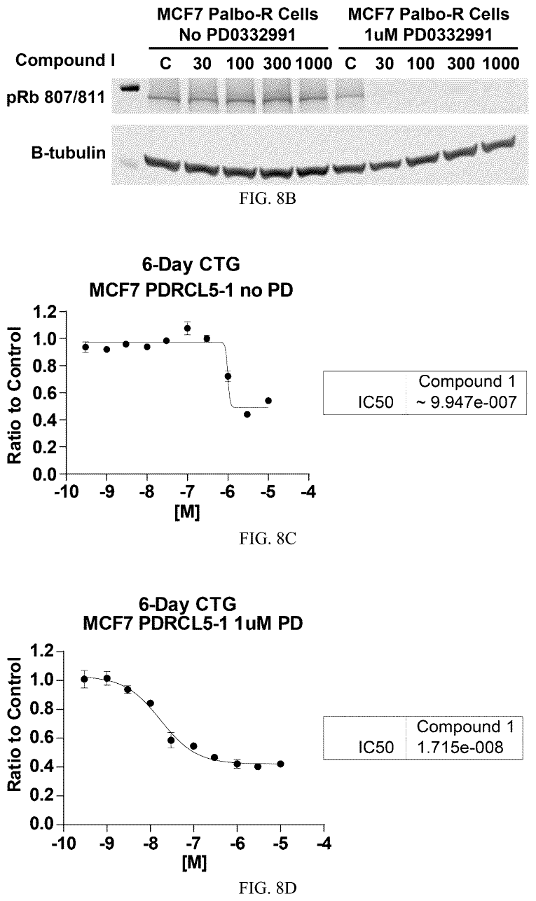

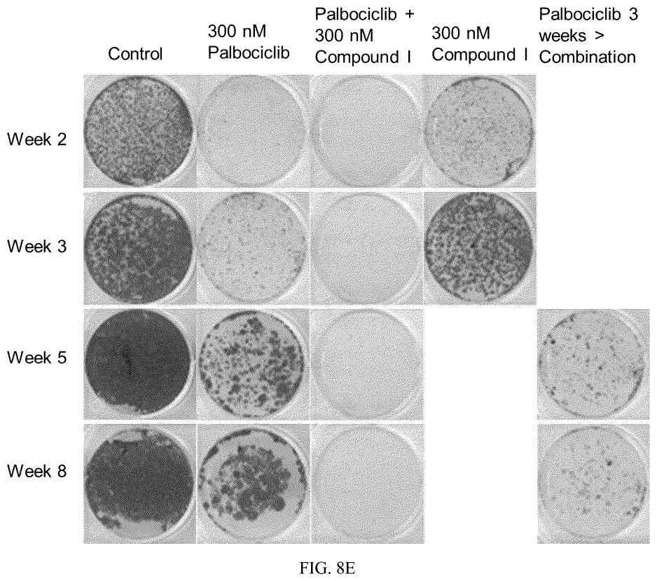

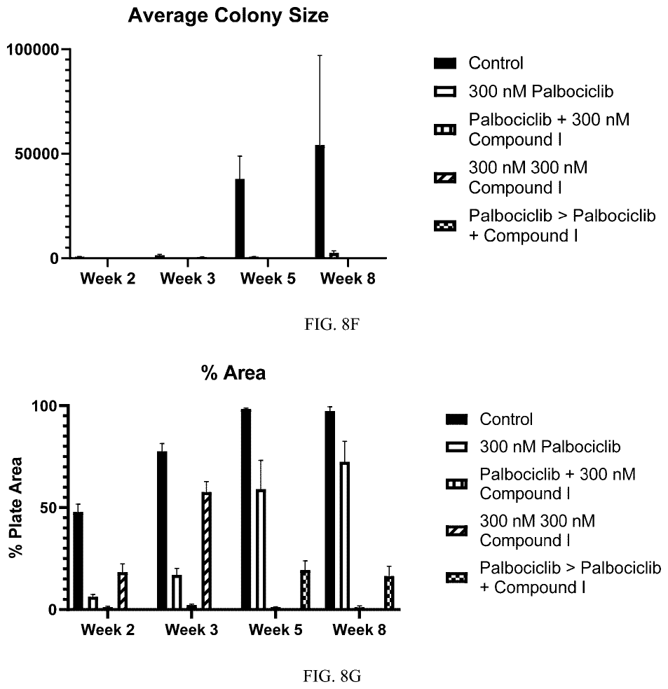

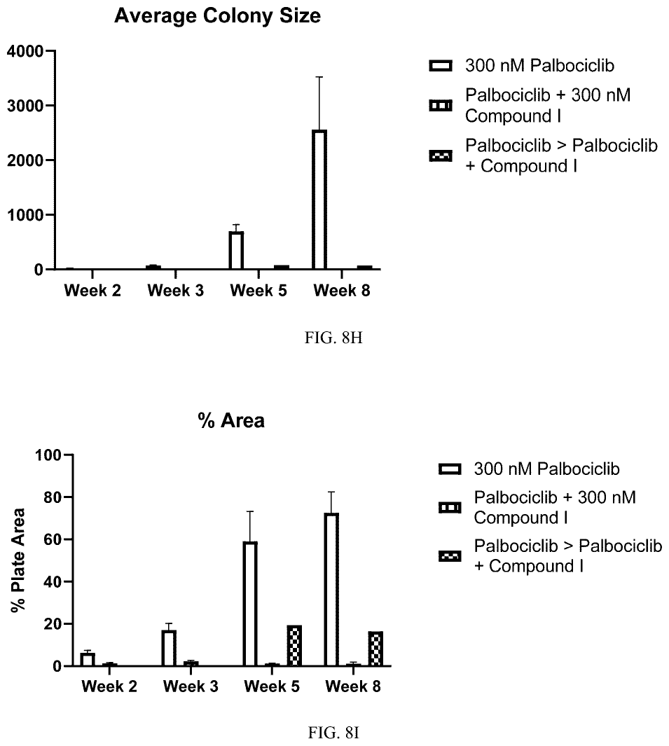

MCF7PDRC15 palbociclib resistant cells were treated with palbociclib, palbociclib plus 100 nM Compound I fixed, Compound I, Compound I plus 1 mM palbociclib fixed as 10-point dose response. Plates were removed from incubator on day six and treated with CellTiter-Glo (Promega, Inc) following manufacturer's specifications. Plates were then read on Biotek luminometer with 1 sec/well integration. Results were converted to Microsoft Excel and transferred to Graphpad Prism for analysis. Cellular viability IC₅₀ values were reached at low nanomolar amounts of Compound I in combination with palbociclib (FIG. 8A).

The MCF7PDRC15-1 palbociclib resistant breast cancer cell line was grown in EMEM/1× Glutamax/1× Insulin/10% fetal bovine serum in the presence or absence of 1 mM palbociclib. Cells were harvested, counted and then plated at 300,000 cells/well in 60 mm tissue culture treated dishes. Cells were incubated at 37° C. for 24 hours prior to addition of Compound I. Treatments conditions included 0, 30 nM, 100 nM, 300 nM, and 1000 nM Compound I. Cells were treated for 24 hours then harvested by washing each well twice with cold PBS, then scraping cells in the presence of 100 μL cold RIPA with protease and phosphatase inhibitors. Lysates were clarified by centrifugation at 12,000×g for 15 min at 4° C. and supernatants were transferred to clean tubes. A BCA assay was performed to determine the protein concentration of each lysate. A total of 25 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBST, membranes were incubated for 60 minutes in LI-COR secondary antibodies. Following 3 washes in TBST, and one TBS wash, membranes were scanned using a LI-COR OdysseyCLx. Image studio was used for image analysis. Increasing concentrations of Compound I led to little reduction in phosphorylated Rb (pRb) levels in Palbo-R MCF7 cells (FIG. 8B). In contrast, when Compound I was co-administered with 1 μM palbociclib, a substantial reduction in pRb levels were observed in Palbo-R MCF7 cells.

The Palbo-R MCF7PDRCL5-1 cells were grown in EMEM, glutamax, 10% FBS, 1×ITS. Cells were plated at 1000 cells/well and treated with Compound I 24 hours later as a 10-point dose response from 0.3 nM to 10 mM in the absence of palbociclib for 72 hours. Plates were removed from incubator on day six and treated with CellTiter-Glo (Promega, Inc) following manufacturer's specifications. Plates were then read on Biotek luminometer with 1 sec/well integration. Results were converted to Microsoft Excel and transferred to Graphpad Prism for analysis. Compound I alone led to a Cell Viability IC₅₀ value of 995 nM (FIG. 8C).

In a similar manner, Palbo-R MCF7PDRCL5-1 cells were grown in EMEM, Glutamax, 10% FBS, 1×ITS. Cells were plated at 1000 cells/well and 24 hours later were treated with Compound I as a 10-point dose response from 0.3 nM to 10 mM in the presence of 1 μM palbociclib. Plates were removed from incubator on day six and treated with CellTiter-Glo (Promega, Inc) following manufacturer's specifications. Plates were then read on Biotek luminometer with 1 sec/well integration. Results were converted to Microsoft Excel and transferred to Graphpad Prism for analysis. The combined administration of Compound I and 1 μM Palbociclib more potently reduced cell viability compared with Compound I administered alone, with Compound I achieving an IC₅₀ value of 17 nM (FIG. 8D).

The T47D human breast carcinoma cell line was grown in RPMI-1640/1× Glutamax/1× Insulin/10% fetal bovine serum and was plated at 10,000 cells/well in Corning 6-well tissue culture treated plates. Cells were incubated at 37° C. for 24 hours prior to addition of treatment. 4 plates were treated with 0.1% DMSO and 300 nM palbociclib in triplicate, 2 plates were treated with combination of 300 nM Compound I and 300 nM palbociclib, or 300 nM Compound I, and 2 plates were treated with combination of 300 nM Compound I and 300 nM palbociclib, or 300 nM palbociclib (FIG. 8E). Plates were collected at week 2 and 3, at week 3, 2 plates with only palbociclib were switched to combination treatment. The remaining plates were collected at weeks 5 and 8. Collection was performed by washing wells with PBS before incubating 5 minutes with 0.1% crystal violet/20% methanol. Dye was removed and wells washed 4 times with PBS before being scanned at 1200 dpi. Image J was used to count the % area and colony size for each well (FIG. 8F-I). GraphPad Prism was used for analysis. Cells treated with DMSO control retained large colonies that inhabited the majority of the well surface area from week 3 beyond (FIG. 8F). Although palbociclib-treated cell colonies were small relative to control condition, cells expanded to cover a large percentage of the plate area. In comparison, Compound I administered alone or in combination with palbociclib led to substantially reduced colony size (FIG. 8H) and percent plate area (FIG. 8I) compared with palbociclib-alone treated cells.

### Palbociclib-Resistant MCF7 Cells are Highly Sensitive to Transient Cell Cycle Arrest by Compound I

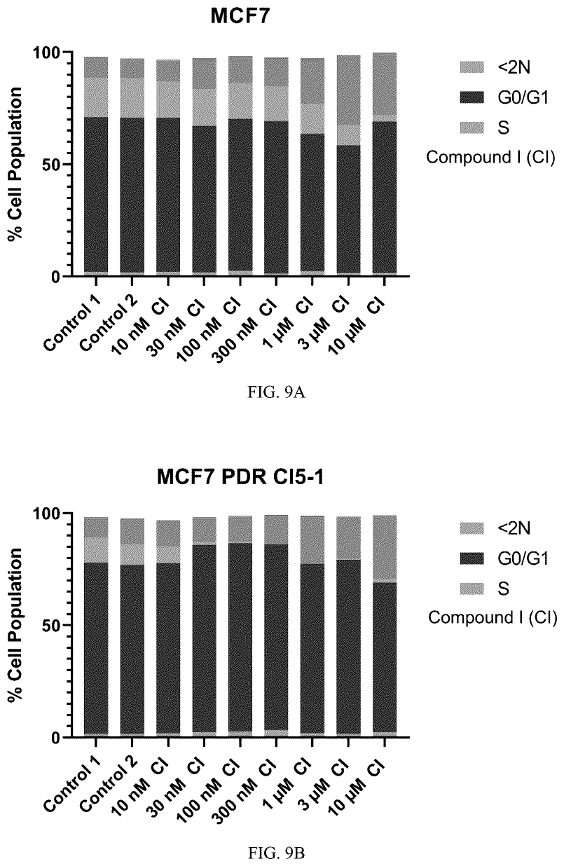

The Palbo-sensitive (Palbo-S) MCF7 parental and Palbo-R daughter cells (MCF7 PDRC15-1) human lung carcinoma cell line was grown in MEM/1× Glutamax/1× Insulin/10% fetal bovine serum with the PDR cells grown in the presence of 1 μM palbociclib. Cells were plated at 100,000 cells/well in Corning 6 cm tissue culture treated plates. Cells were incubated at 37° C. for 24 hours prior to addition of 0.1% DMSO control or 10 nM to 10 μM Compound I. MCF7 PDR cells were also treated with 1 μM palbociclib. Cells were treated for 24 hours and 10 μM EdU was added 1 hour prior to collection. Cells were collected and fixed in 1% paraformaldehyde. Cells were incubated with Alexa488 azide dye as described by Thermo Scientific. Cells were then incubated with FarRed dye and RNase. Cells were analyzed on a BD Celesta and data compiled using FloJo and GraphPad. Palbo-S MCF7 cells are not sensitive to Compound I and a G2 arrest begins to occur at 1 μM and is stronger at 10 μM (FIG. 9A). In contrast, Palbo-R MCF7 cells are more sensitive to Compound I, with cells becoming arrested during the G1 phase at 30, 100, 300 nM concentrations of Compound I (FIG. 9B). At Compound I concentrations of 1 μM and greater there is a G2 arrest of Palbo-R MCF7 cells. The resistant Palbo-R MCF7 cells have a much lower percent of cells in S phase in general.

### Example 4. Compound I Potently Inhibits CCNE^{high} Cancers

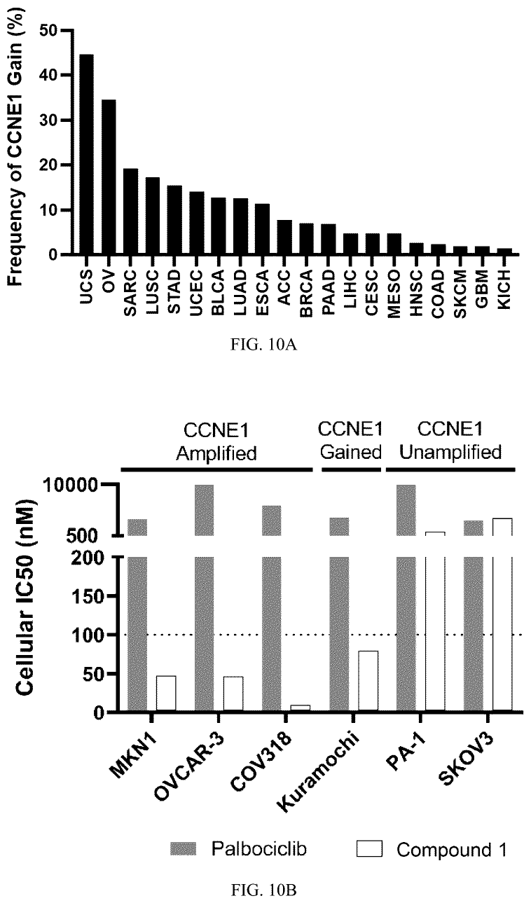

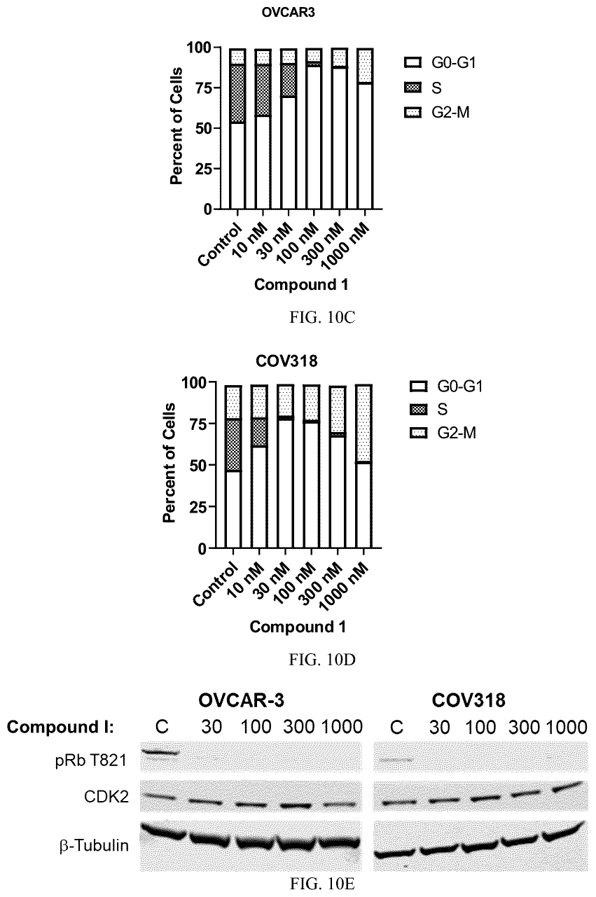

Cancers in which cyclin E is overexpressed and/or amplified represents a patient population of approximately 100,000 individuals (Cerami et al. Cancer Discov. 2(5): 401-4(2012); Gao et al. Sci Signal. 6(269): pl1(2013); TCGA Research Network). In many cancers, over 10% of tumors on average exhibit gained or amplified cyclin E1 (CCNE1) expression (FIG. 10A). For example, gene amplification is observed in approximately 20% of ovarian high grade serous carcinoma (Kanska et al. Gynecol Oncol. 143(1): 152-8(2016)). Cyclin E amplification in ovarian cancer is associated with chemotherapy resistance and poor prognosis (Farley et al. Cancer Res. 63:1235-41(2003); Sui et al. Gynecol Oncol. 83:56-63(2001)).

A representative collection of ovarian cancer cell lines that are either cyclin E amplified, gain of function, or unamplified cancers were assessed in CellTiter Glo cell proliferation assay comparing the CDK4/6 inhibitor palbociclib and the CDK2 inhibitor Compound I. The OVCAR-3, COV318 and Kuramochi cells were plated at 500 cells per well, while PA-1 cells were plated at 50 cells per well, and SKOV3 cells were plated at 1,000 cells/well. Twenty-four hours later, cells were dosed with Compound I or palbociclib as a ten-point dose curve from 300 pM to 10 mM with 10× drug stocks. Plates were returned to the incubator for six additional days. On day six post dosing, plates were removed and treated with CellTiter Glo reagent following manufacturer's specifications. Plates were incubated 45 minutes at room temperature and run on Biotek luminometer with 1 sec/well detection. Results were converted to Microsoft Excel and transferred to Graphpad Prism for analysis. When compared with palbociclib, Compound I potently decreases cell viability in several ovarian cancer model cells (OVCAR-3, COV318, Kuramochi) at low nanomolar concentrations (FIG. 10B). These cell types are distinguished by either gained of cyclin E1 expression or activation (Kuramochi) or intrinsic amplification of cyclin E1 (OVCAR-3, COV318). In ovarian cancer model cells lacking cyclin E amplification (PA-1, SKOV3), Compound I inhibition is comparable to palbociclib alone. These results demonstrate that the selective CDK2 inhibitor Compound I retains potency in cells with amplified cyclin E1 expression and/or activation.

To assess cell cycle changes associated with the administration of Compound I, the ovarian carcinoma cell line OVCAR-3 grown in RPMI/1× Glutamax/1×ITS/10% fetal bovine serum, while the COV318 cells were grown in the presence of DMEM/1× Glutamax/10% FBS. Cells were plated at 100,000 cells/well in Corning 6 cm tissue culture treated plates. Cells were incubated at 37° C. for 24 hours prior to addition of 0.1% DMSO control or 10 nM to 10 μM Compound I. Cells were treated for 24 hours and 10 μM EdU was added 1 hour prior to collection. Cells were collected and fixed in 1% paraformaldehyde. Cells were incubated with Alexa488 azide dye as described by Thermo Scientific. Cells were then incubated with Fxcycle-violet and RNase. Cells were analyzed on a BD Celesta and data compiled using FloJo and GraphPad. OVCAR-3 and COV318 cells incubated with increasing concentrations of Compound I exit the S-phase and increasingly occupy the G0-G1 phase (FIG. 10C-D).

### Compound I Inhibits Phosphorylated Retinoblastoma Protein in a Dose- and Time-Dependent Manner

To assess whether the phosphorylation status of Rb protein is altered by the administration of Compound I, the ovarian carcinoma cell line OVCAR-3 grown in RPMI/1× Glutamax/1× ITS/10% fetal bovine serum, while the COV318 cells were grown in the presence of DMEM/1× Glutamax/10% FBS. Cells were plated at 300,000 cells/well in Corning 6 cm tissue culture treated plates. Cells were incubated at 37° C. for 24 hours prior to addition of 0.1% DMSO control or 30 nM, 100 nM, 300 nM and 1000 nM Compound I. Cells were treated for 24 hours. Cells were collected by washing each well twice with cold PBS, then scraping cells in the presence of 75 μL cold RIPA with protease and phosphatase inhibitors. Lysates were clarified by centrifugation at 12,000×g for 15 min at 4° C. and supernatants transferred to clean tubes. A BCA assay was performed to determine the protein concentration of each lysate. A total of 25 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBS-T, membranes were incubated for 60 minutes in LI-COR secondary antibodies. Following 3 washes in TBS-T, and one TBS wash, membranes were scanned using a LI-COR OdysseyCLx. Image studio was used for image analysis. Increased concentrations of Compound I administered to ovarian cancer model OVCAR-3 and COV318 cells led to a dose-dependent decrease in pRb levels, assayed by immunolabeling using an antibody anti-pT821 Rb (FIG. 10E).

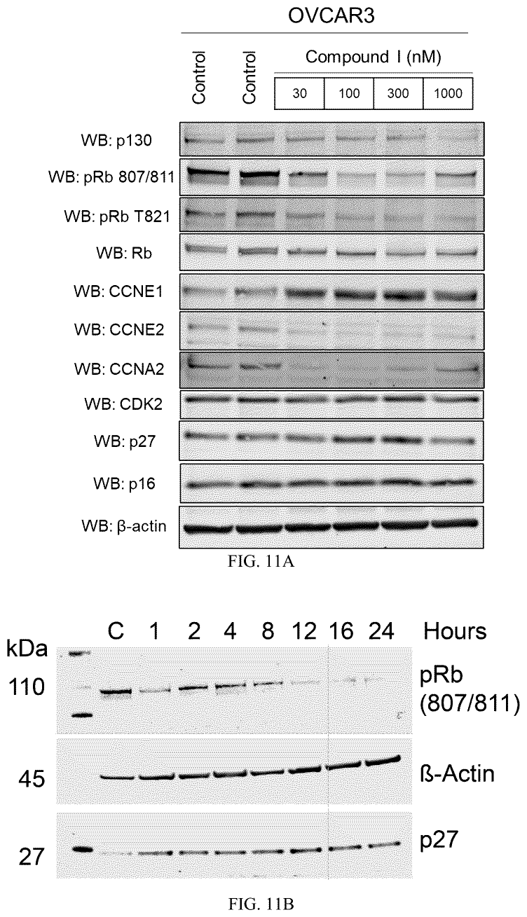

The OVCAR-3 human ovarian carcinoma cell line was grown in RPMI-1640/1× Glutamax/1× Insulin/10% fetal bovine serum and was plated at 300,000 cells/well in Corning 6 well tissue culture treated plates. Cells were incubated at 37° C. for 24 hours prior to addition of 2 replicates of 0.1% DMSO control and 1 replicate for each treatment. Treatments were 30 nM Compound I, 100 nM Compound I, 300 nM Compound I, 1000 nM Compound I. Cells were treated for 24 hours. Cells were collected by washing each well twice with cold PBS, then scraping cells in the presence of 150 μL cold RIPA with protease and phosphatase inhibitors. Lysates were clarified by centrifugation at 14,000×g for 10 min at 4° C. and supernatants were transferred to clean tubes. A BCA assay was performed to determine the protein concentration of each lysate. A total of 30 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBS-T, membranes were incubated for 45 minutes in LI-COR secondary antibodies. Following 2 washes in TBS-T, and one TBS wash, membranes were scanned using a LI-COR OdysseyCLx. Image studio was used for image analysis. (FIG. 11A-B). Compound 1 induced a dose-dependent decrease in pRb levels (FIG. 11A). Interestingly, cyclin E1 (CCNE1) levels were increased with the administration of Compound I.

As a substantial decrease in pRb levels were observed after incubating OVCAR-3 cells with Compound I, different incubation lengths were tested to observe how quickly pRb levels may be altered with the administration of Compound I. The ovarian carcinoma cell line OVCAR-3 was grown in RPMI/1× Glutamax/1×ITS/10% fetal bovine serum. Cells were plated at 300,000 cells/well in Corning 6 cm tissue culture treated plates. Cells were incubated at 37° C. for 24 hours prior to addition of 0.1% DMSO control or 300 nM Compound I. Cells were treated for 1, 2, 4, 8, 12, 16 and 24 hours. Cells were collected by washing each well twice with cold PBS, then scraping cells in the presence of 75 μL cold RIPA with protease and phosphatase inhibitors. Lysates were clarified by centrifugation at 12 kxg for 15 min at 4° C. and supernatants transferred to clean tubes. A BCA assay was performed to determine the protein concentration of each lysate. A total of 20 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBST, membranes were incubated for 60 minutes in LI-COR secondary antibodies. Following 3 washes in TBST, and one TBS wash, membranes were scanned using a LI-COR OdysseyCLx. Image studio was used for image analysis. The administration of Compound I for increasing lengths of time induced a time-dependent decrease in pRb levels (FIG. 11B). A substantial reduction in pRb levels were observed starting post-8 hours incubation with Compound 1, further decreasing at 16 hour and 24 hour incubation lengths.

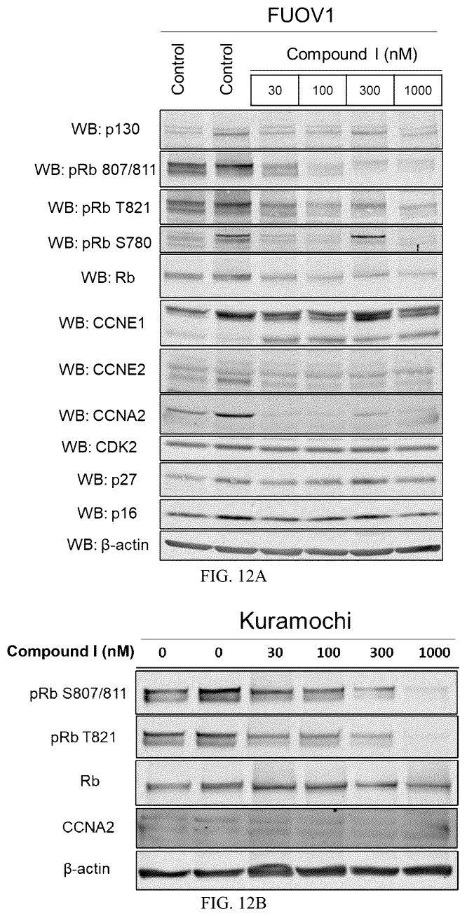

An independent human ovarian carcinoma cell line, FUOV-1, was grown in DMEM/F12/1× Glutamax/10% fetal bovine serum and plated at 300,000 cells/well in Corning 6 well tissue culture treated plates. Cells were incubated at 3° C. for 24 hours prior to addition of 2 replicates of 0.1% DMSO control and 1 replicate for each treatment. Treatments were 30 nM Compound I, 100 nM Compound I, 300 nM Compound I, 1000 nM Compound I. Cells were treated for 24 hours. Cells were collected by washing each well twice with cold PBS, then scraping cells in the presence of 150 μL cold RIPA with protease and phosphatase inhibitors. Lysates were clarified by centrifugation at 14,000×g for 10 min at 4° C. and supernatants transferred to clean tubes. A BCA assay was performed to determine the protein concentration of each lysate. A total of 30 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBS-T, membranes were incubated for 45 minutes in LI-COR secondary antibodies. Following 2 washes in TBS-T, and one TBS wash, membranes were scanned using a LI-COR OdysseyCLx. Image studio was used for image analysis. Compound I decreases pRb levels in a dose-dependent manner in ovarian cancer model FUOV1 cells (FIG. 12A). In contrast to OVCAR-3 cells, no cyclin E amplification was observed with increasing Compound I concentrations, however.

An independent human ovarian carcinoma cell line, Kuramochi human ovarian carcinoma, was grown in RPMI-1640/1× Glutamax/10% fetal bovine serum and was plated at 300,000 cells/well in Corning 6 well tissue culture treated plates. Cells were incubated at 37° C. for 24 hours prior to addition of 2 replicates of 0.1% DMSO control and 1 replicate for each treatment. Treatments were 30 nM Compound I, 100 nM Compound I, 300 nM Compound I, 1000 nM Compound I. Cells were treated for 24 hours. Cells were collected by washing each well twice with cold PBS, then scraping cells in the presence of 150 μL cold RIPA with protease and phosphatase inhibitors. Lysates were clarified by centrifugation at 14 kxg for 10 min at 4° C. and supernatants transferred to clean tubes. A BCA assay was performed to determine the protein concentration of each lysate. 30 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBST, membranes were incubated for 45 minutes in LI-COR secondary antibodies. Following 2 washes in TBST, and one TBS wash, membranes were scanned using a LI-COR OdysseyCLx. Image studio was used for image analysis. Compound I decreases pRb levels in a dose-dependent manner in ovarian cancer model Kuramochi cells (FIG. 12B).

Compound I Induces Enhanced Percent of G1-Arrested CCNE^{high} Ovarian OVCAR-3 Cells Compared with Other CDK Inhibitors

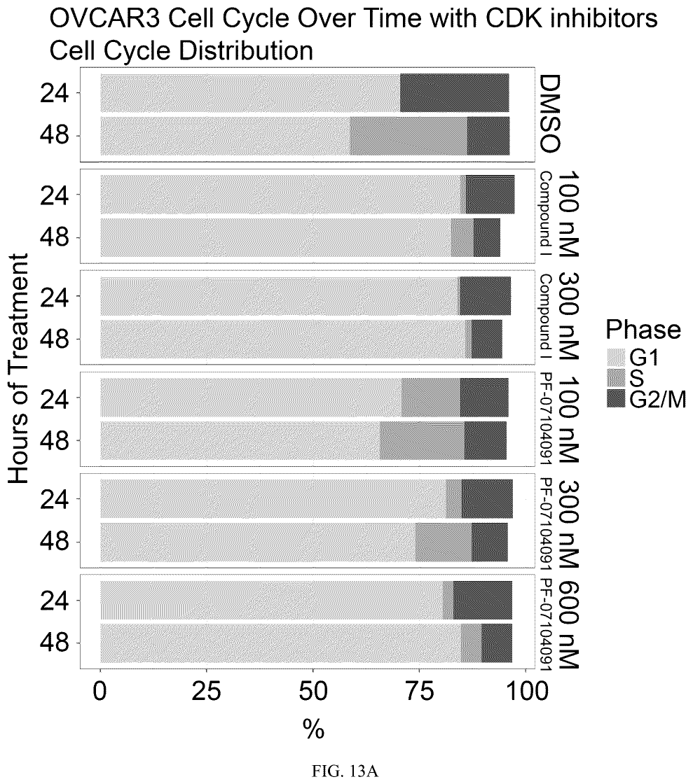

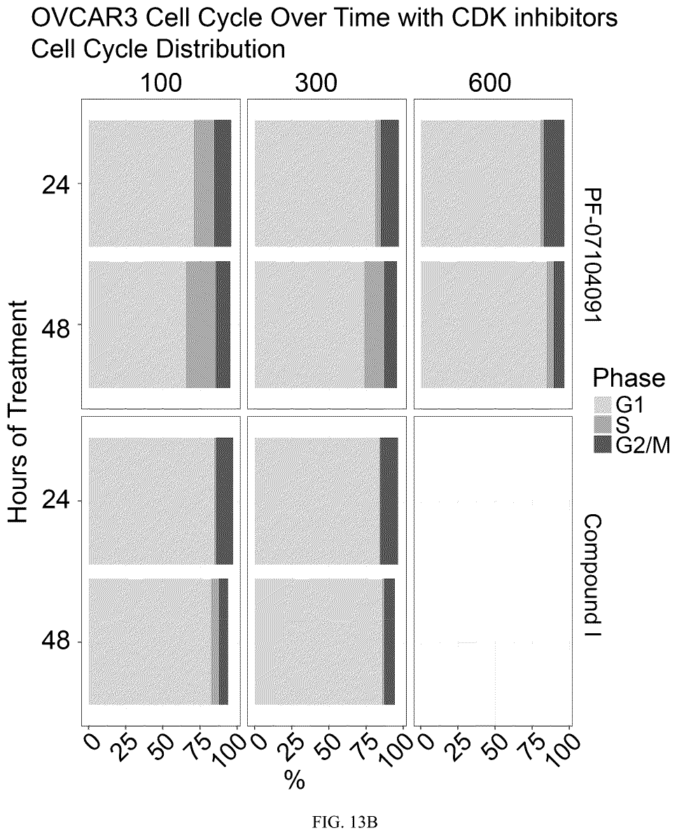

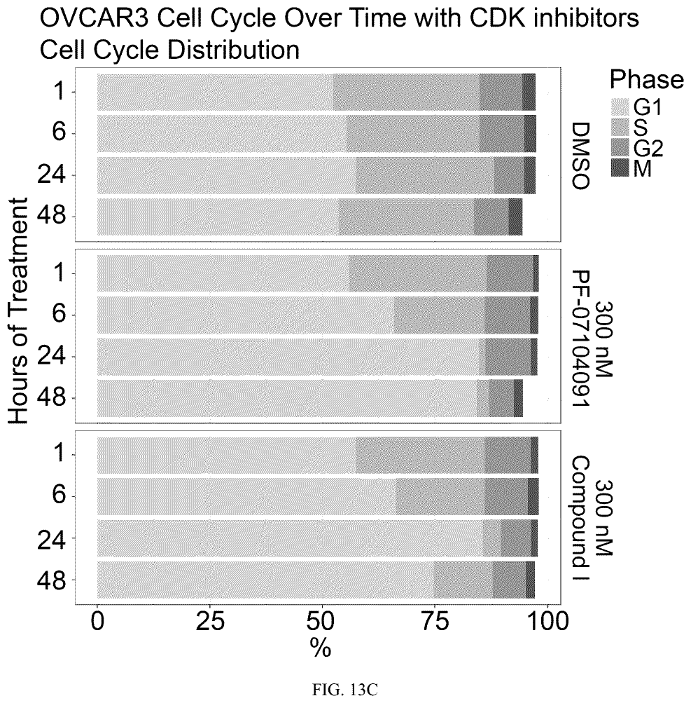

The OVCAR3 human ovarian carcinoma cell line was grown in RPMI-1640/1× Glutamax/1× Insulin/10% fetal bovine serum and was plated at 300,000 cells/well in Corning 6 cm tissue culture treated plates. Cells were incubated at 37° C. for 24 hours prior to addition of 0.1% DMSO control, or the indicated concentration of Compound I or PF-07104091. Cells were treated for the indicated amount of time (24 hours or 48 hours), with 10 μM EdU added for the last hour of treatment. Cells were collected and fixed in 1% paraformaldehyde. Cells were incubated with Alexa 488 (A488) azide dye as described by Thermo Scientific. Cells were then incubated with FarRed dye and RNase. Cells were analyzed on a BD Celesta and data compiled using FloJo and GraphPad Prism (FIG. 13A-B). No A488 signal was observed for the DMSO control treatment at 24 hr (FIG. 13A). When administered at a concentration of 100 nM, Compound I does not arrest all cells in G1 but permits a small percent of cells through G₁ at 48 h. At a concentration of 600 nM, PF-07104091 appears to hold cells in G1 over 48 hours, whereas 300 nM Compound I performs just as well (FIG. 13B).

A further cell cycle analysis was conducted for OVCAR cells treated with Compound I and PF-07104091 for 1, 6, 24, and 48 hours (FIG. 13C). Compound I permits fewer cells to proceed to S phase over 48 hours of treatment when compared with PF-07104091.

### Compound I Induces Rapid and Transient Cell Cycle Arrest in CCNE^{high} Ovarian OVCAR-3 Cells

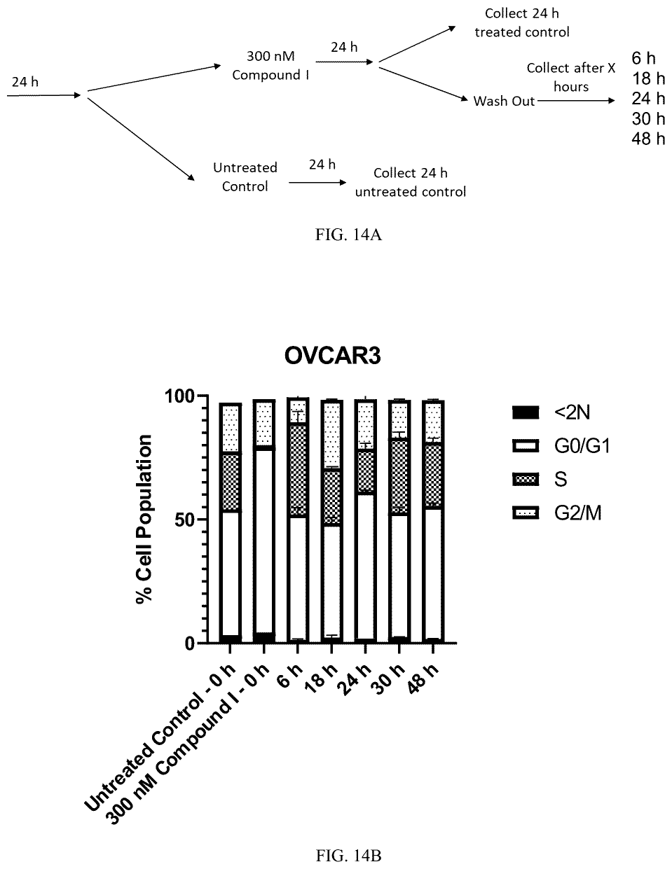

The OVCAR3 human ovarian carcinoma cell line was grown in RPMI-1640/1× Glutamax/1× Insulin/10% fetal bovine serum and was plated at 300,000 cells/well in Corning 6 cm tissue culture treated plates. Cells were incubated at 37° C. for 24 hours prior to addition of 0.1% DMSO control, or 300 nM Compound I. After 24 hours, the untreated plate and one treated plate were collected, with 10 μM EdU added for the last hour. At 24 hours, the remaining plates were rinsed with media before un-treated media was added back. Cells were collected at the indicated times, with 10 μM EdU added for the last hour (FIG. 14A). Cells were collected and fixed in 1% paraformaldehyde. Cells were incubated with Pacific blue azide dye as described by Thermo Scientific. Cells were then incubated with FarRed dye and RNase. Cells were analyzed on a BD Celesta and data compiled using FloJo and GraphPad. Approximately 80% of OVCAR-3 cells were arrested in G₁ at 24 hours (FIG. 14B). A large portion of cells were in early S phase 6 hours after Compound I was washed out. By 18 hours, OVCAR-3 cells were in G2 or reentering G1. Cells appeared to remain somewhat synchronized over 48 hours.

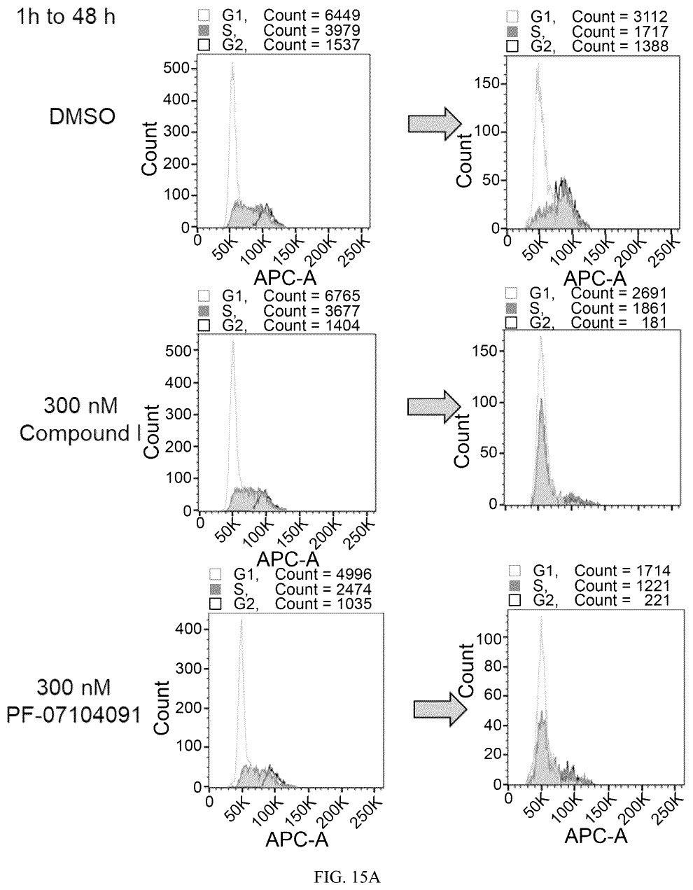

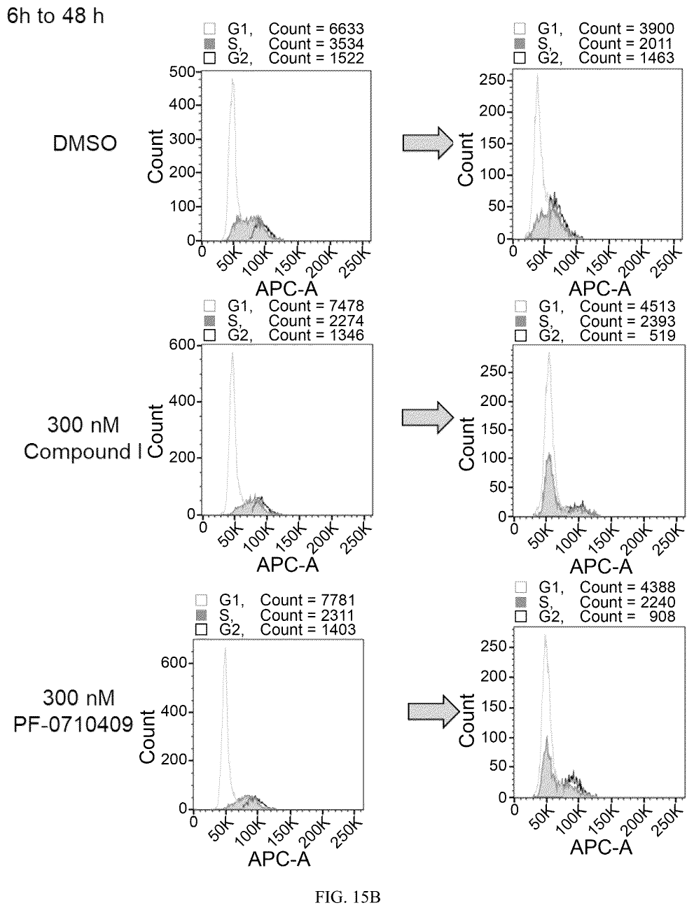

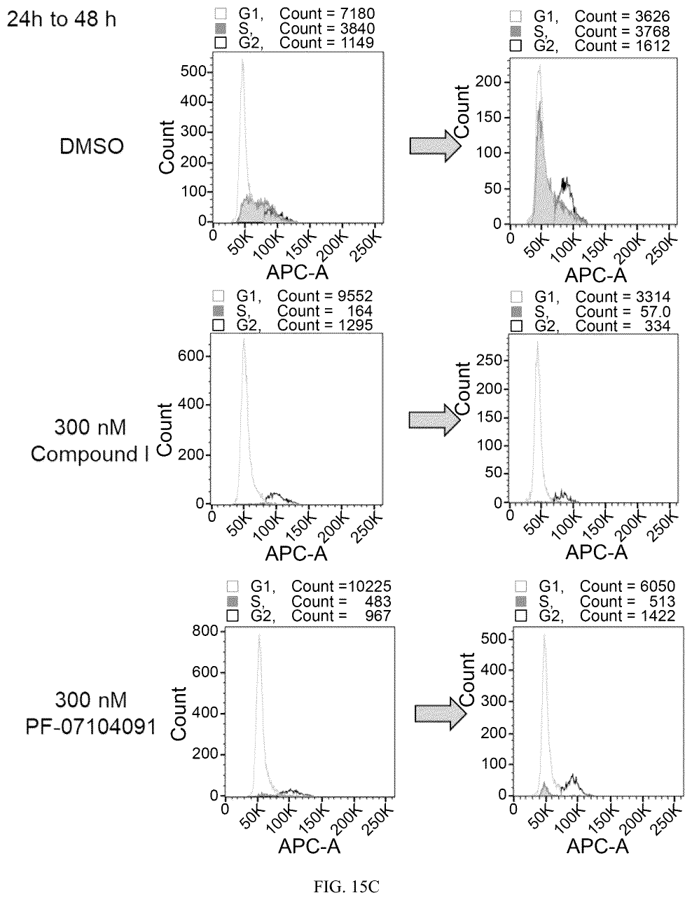

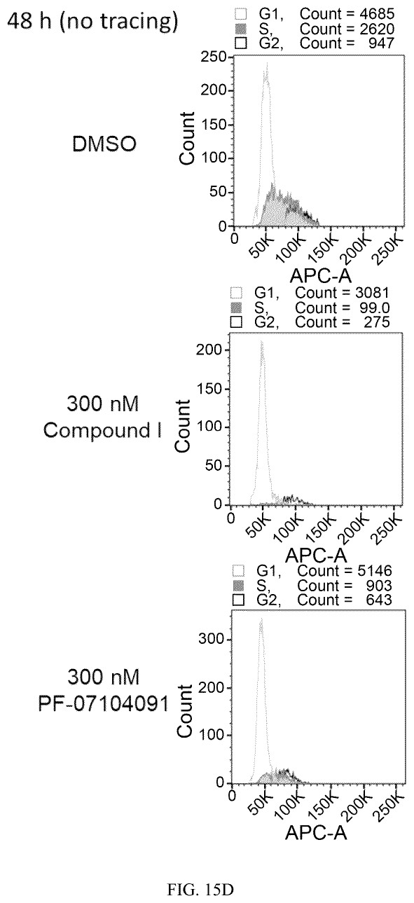

The OVCAR3 human ovarian carcinoma cell line was grown in RPMI-1640/1× Glutamax/1× Insulin/10% fetal bovine serum and was plated at 300,000 cells/well in Corning 6 cm tissue culture treated plates. Cells were incubated at 37° C. for 24 hours prior to addition of 0.1% DMSO control, or the indicated concentration of Compound I or PF-07104091. Cells were divided into two sets. The first set was treated for the indicated amount of time, with 10 μM EdU added for the last hour of treatment before being collected. The second set of cells were treated for the indicated amount of time and had EdU added for one hour before the timepoint. Then the media was aspirated, the cells washed, and re-treated with treatment media. The second set of cells was collected at 48 hours, allowing for tracing of the EdU positive cells. Cells were collected and fixed in 1% paraformaldehyde. Cells were incubated with Alexa 488 (A488) azide dye as described by Thermo Scientific. Cells were then incubated with FarRed dye and RNase. Cells were analyzed on a BD Celesta and data compiled using FloJo and GraphPad. Cells were traced to identify where replicating cells arrested following treatment with Compound I (FIG. 15A-D). Several “snap shots” of cells at various timepoints, followed by where the cells from S phase ended up at the end of 48 hours of treatment, were collected. The “S phase” cells in the traced samples reveals the final distribution of cells that were in S phase when EdU was present. It appears that the cells in S phase at treatment initiation become arrested in G1 (FIG. 15A). It appears that the cells in S phase at after 6 hours of treatment are arresting in G1 (FIG. 15B). At 24 hours of treatment, little to no cells were in S phase so there were no cells to trace (FIG. 15C). Although the DMSO sample appears largely different at 48 hours, the distribution appears similar to the DMSO, EdU free cells at 48 hours. These results indicate that EdU did not impact cell growth. (FIG. 15D).

Compound I Exhibits Greater Inhibition of CCNE^{high} Ovarian Cells Compared with Other CDK Inhibitors

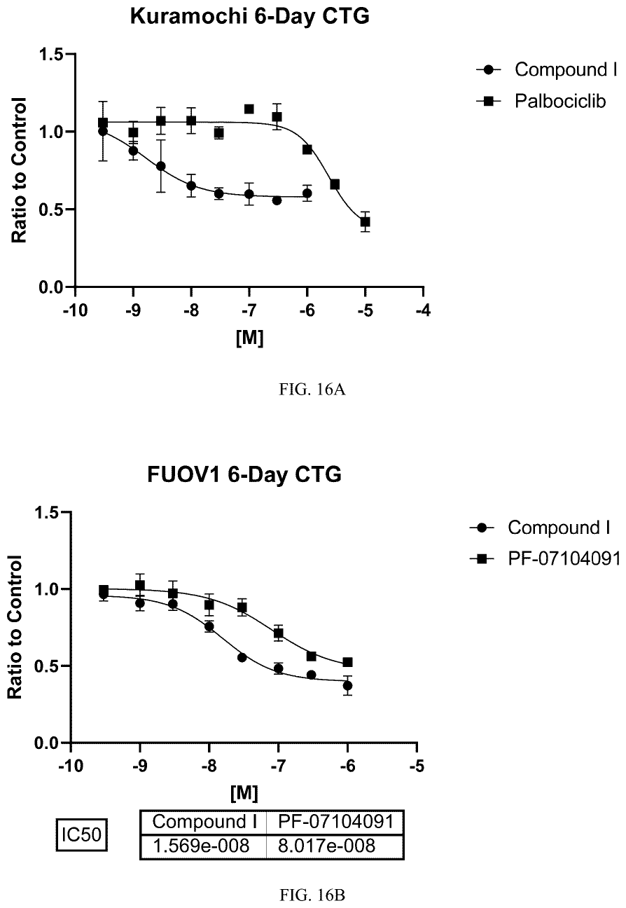

The Kuramochi human ovarian carcinoma cell line was grown in RPMI-1640/1× Glutamax/10% fetal bovine serum and the FUOV-1 human ovarian carcinoma cell line was grown in DMEM/F12/1× Glutamax/10% fetal bovine serum and both were plated at 1,000 cells/well in Corning 96 well white wall/clear bottom tissue culture treated plates. Cells were incubated at 37° C. for 24 hours prior to addition of Compound I or PF-07104091 from 300 pM to 10 μM in triplicate for 6 days. Plates were removed from incubator at each specified timepoint and treated with CellTiter-Glo (Promega, Inc) following manufacturer specifications. Plates were then read on CLARIOstar luminometer with 1 sec/well integration. Results were converted to Microsoft Excel and transferred to GraphPad Prism for Analysis (FIG. 16A-B). Compound I inhibited ovarian carcinoma Kuramochi cells at concentrations substantially lower than palbociclib (FIG. 16A). Similarly, Compound I demonstrated lower IC₅₀ values in ovarian carcinoma FUOV1 cells (16 nM) than PF-07104091 (80 nM) (FIG. 16B).

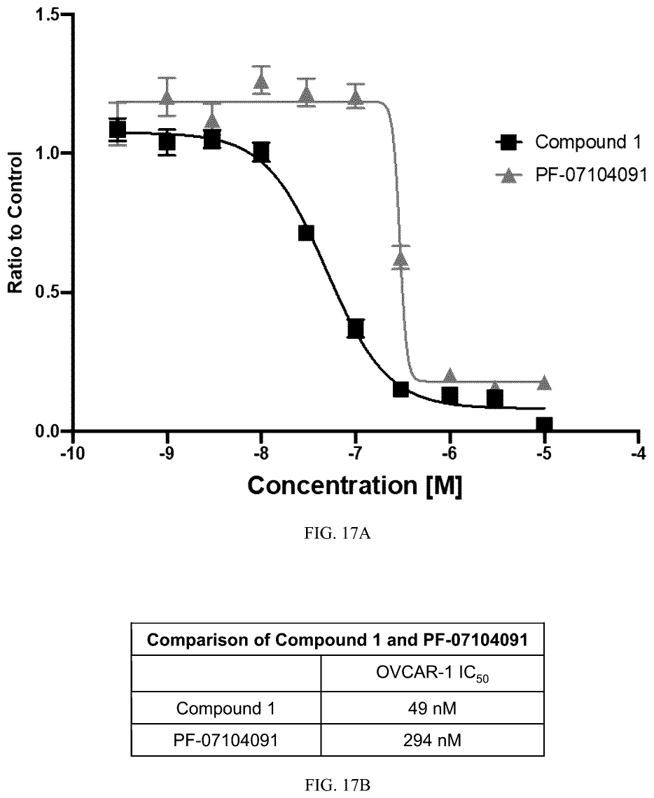

In a similar manner, OVCAR-3 cells were grown in RPMI-1640, 1× Glutamax, 10% FBS and 1×ITS at 37° C., 5% CO2. Cells were plated at 500 cells per well in white wall/clear bottom plates and incubated overnight. Twenty-four hours later, cells were dosed with Compound I or PF-07104091 as a ten-point dose curve from 300 pM to 10 mM with 10× drug stocks. Plates returned to incubator for six additional days. On day six post dosing, plates were removed and treated with CellTiter Glo reagent following manufacturer's specifications. Plates were incubated 45 minutes at room temperature and run on Biotek luminometer with 1 sec/well detection. Results were converted to Microsoft Excel and transferred to GraphPad Prism for analysis. Compound I inhibited ovarian carcinoma OVCAR-3 cells at lower concentrations than PF-07104091 (FIG. 17A). Compound I exhibited a 6-fold lower cell proliferation IC₅₀ value (49 nM) in OVCAR-3 cells than PF-07104091 (294 nM) (FIG. 17B).

### Compound I Reduces Tumor Volume and Extends Survival in OVCAR-3 Implanted Mice

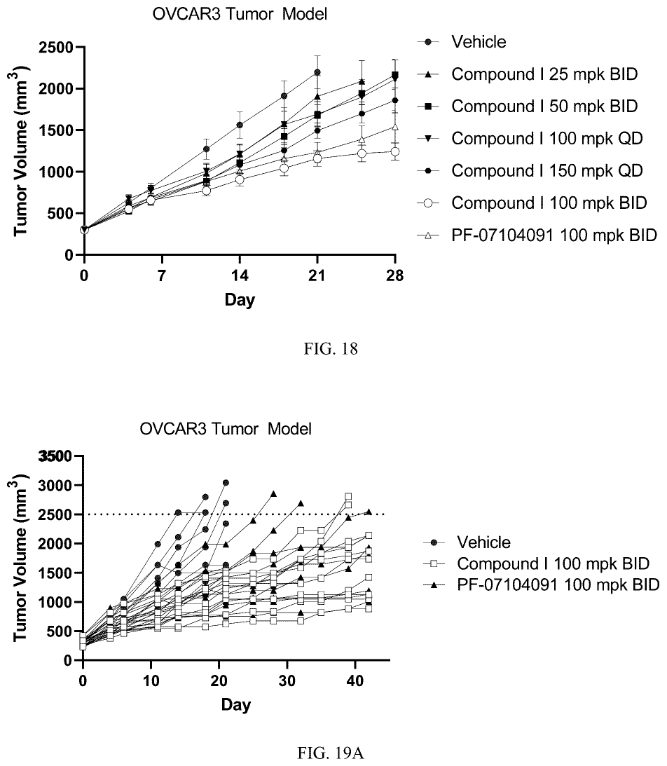

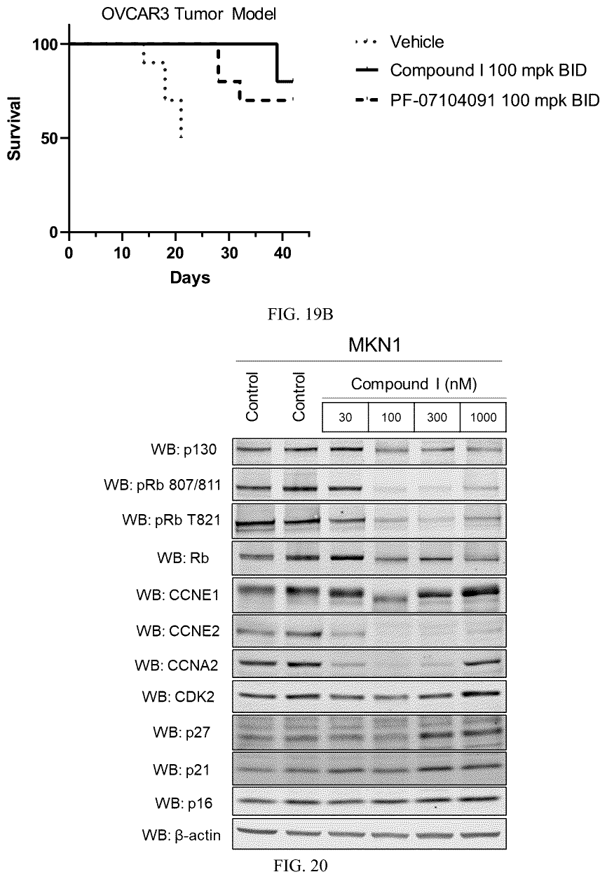

OVCAR-3 xenograft tumor fragments were harvested from host animals and implanted into immune-deficient mice (CR1:NU(NCr)-Fox1^{nu}). The study was initiated at a mean tumor volume of approximately 175-300 mm³. Compound I was dosed at 25, 50 and 100 milligrams per kilogram (mpk) BID, as well as 100 and 150 mpk QD. PF-07104091 was dosed at 100 mpk BID. The test agents were delivered at 10 mL/kg by oral gavage. These results show tumor growth inhibition (TGI) out to 28 days post dose initiation. Compound I administered twice daily at 100 mpk reduced tumor volume the most among all treatment conditions (FIG. 18). Individual replicate values over the course of the experiment are shown in FIG. 19A, extended out to 42 days post-study initiation. A Kaplan-Meyer survival curve representation of individual animal survival comparing Compound I and PF-07104091 at 100 mpk BID from OVCAR-3 xenograft study as described in FIG. 19A was performed. The group of mice administered Compound I twice daily at 100 mpk exhibited the greatest overall survival, exceeding that of mice administered PF-07104091 at 100 mpk twice a day (FIG. 19B). Mice xenotransplanted with OVCAR-3 tumor cells and administered vehicle exhibited the worst survival outcomes.

Compound I Inhibits Phosphorylated Retinoblastoma (pRb) Protein in Gastric Cancer Model MKN1 Cells

The MKN1 human stomach carcinoma cell line was grown in RPMI-1640/1× Glutamax/10% heat inactivate fetal bovine serum and was plated at 300,000 cells/well in Corning 6 well tissue culture treated plates. Cells were incubated at 37° C. for 24 hours prior to addition of 2 replicates of 0.1% DMSO control and 1 replicate for each treatment. Treatments were 30 nM Compound I, 100 nM Compound I, 300 nM Compound I, 1000 nM Compound I. Cells were treated for 24 hours. Cells were collected by washing each well twice with cold PBS, then scraping cells in the presence of 150 μL cold RIPA with protease and phosphatase inhibitors. Lysates were clarified by centrifugation at 14,000×g for 10 min at 4° C. and supernatants transferred to clean tubes. A BCA assay was performed to determine the protein concentration of each lysate. A total of 30 μg of protein was mixed with sample buffer and reducing agent before being run on a 4-12% SDS-PAGE. Protein was transferred to a nitrocellulose membrane and blocked with LI-COR blocking buffer for 1 hr. Membranes were incubated overnight in primary antibody at 4° C. Following 3 washes with TBS-T, membranes were incubated for 45 minutes in LI-COR secondary antibodies. Following 2 washes in TBS-T, and one TBS wash, membranes were scanned using a LI-COR OdysseyCLx. Image studio was used for image analysis. Compound I induced a dose-dependent response decrease in pRb levels in MKN1 cells (FIG. 20). Certain cell cycle inhibitor proteins (p21, p27) and markers of DNA damage (phosphorylated CHK2) were increased with increased doses of Compound I.

### Compound I Induces Rapid and Transient Cell Cycle Arrest in Gastric Cancer Model MKN1 Cells

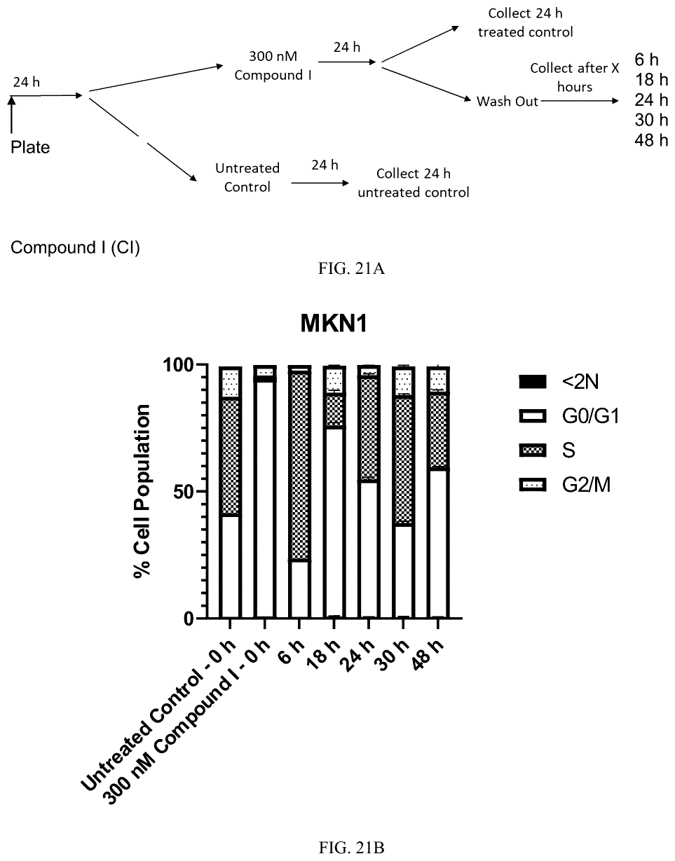

The MKN1 human stomach carcinoma cell line was grown in RPMI-1640/1× Glutamax/10% heat inactivated fetal bovine serum and was plated at 300,000 cells/well in 6 cm tissue culture treated plates. Cells were incubated at 37° C. for 24 hours prior to addition of 0.1% DMSO control, or 300 nM Compound I. After 24 hours, the untreated plate and one treated plate were collected, with 10 μM EdU added for the last hour. At 24 hours, the remaining plates were rinsed with media before un-treated media was added back. Cells were collected at the indicated times, with 10 μM EdU added for the last hour (FIG. 21A). Cells were collected and fixed in 1% paraformaldehyde. Cells were incubated with Pacific blue azide dye as described by Thermo Scientific. Cells were then incubated with FarRed dye and RNase. Cells were analyzed on a BD Celesta and data compiled using FloJo and GraphPad. Approximately 95% of MKN1 cells were arrested at G1 at 24 hr post-initial treatment (0 hr after washout) (FIG. 21B). A large portion of cells were in early S phase 6 hours following the washout of Compound I. Approximately 18 hr following the washout of Compound I, MKN1 cells were mostly in G1. At 48 hr post-washout, MKN1 cells were synchronized.

### Example 5. Compound I Specifically Inhibits Multiple Cell Line Models of Rb-Independent Cancers

Inactivation of retinoblastoma (Rb) protein is suggested to occur in a majority of all cancers (Viatour et al. Dis Model Mech. 4(5): 581-5(2011)). Among this group of disorders includes small cell lung cancer (SCLC), a cancer that is universally Rb-null. Mutations that drive SCLC progression include loss of function of tumor suppressor gene retinoblastoma 1 (RB1) (George et al. Nature. 524(7563): 47-53(2015)). While SCLC cancers initially respond to cisplatin/etoposide combination therapy treatments, the tumors become unresponsive and survival benefits are short-lived. Although comprising approximately 15% of all lung cancers (Kalemkerian et al. J Natl Compr Canc Netw. 11(1): 78-98(2013)), there exists no targeted therapy for SCLC. Currently there are no targeted therapies approved for the treatment of SCLC.

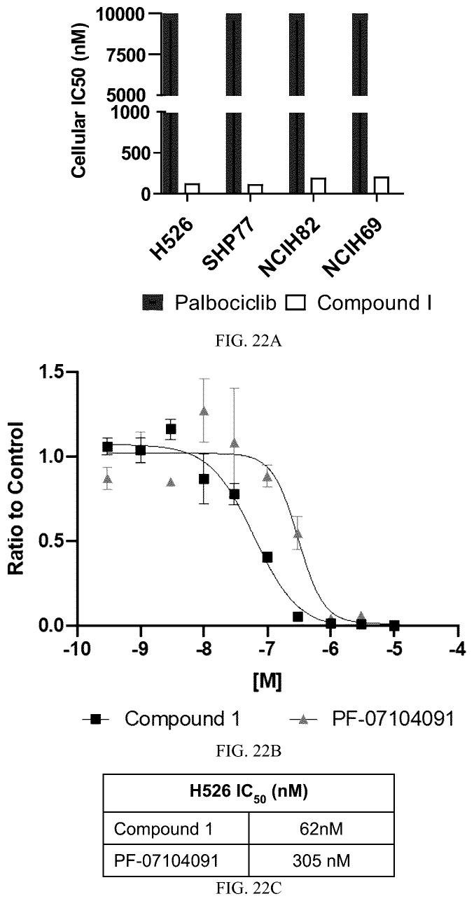

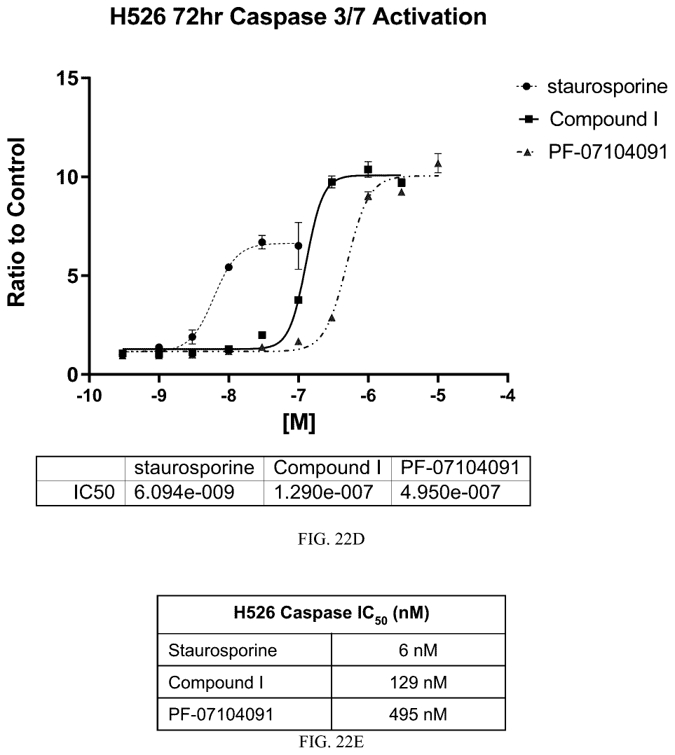

A representative collection of small cell lung cancer cell lines that are retinoblastoma (Rb) independent cancers were assessed in a CellTiter Glo cell proliferation assay comparing the CDK4/6 inhibitor palbociclib and the CDK2 inhibitor Compound I. SHP77, H69 and H82 cells were plated at 3,000 cells/well, whereas H526 cells were plated at 1,000 cells/well. Twenty-four hours later, cells were dosed with Compound I or PF-07104091 as a ten-point dose curve from 300 pM to 10 mM with 10× drug stocks. Plates returned to incubator for six additional days. On day six post dosing, plates were removed and treated with CellTiter Glo reagent following manufacturer's specifications. Plates incubated 45 minutes at room temperature and run on a Biotek luminometer with 1 sec/well detection. Results were converted to Microsoft Excel and transferred to GraphPad Prism for analysis. When compared with palbociclib, Compound I strongly inhibits several independent cell line models of SCLC (H526, SHP77, NCIH82, NCIH69) at low nanomolar concentrations (FIG. 22A).

To compare Compound I to another CDK2 inhibitor, PF-07104091, H526 cells were grown in RPMI-1640, 1× Glutamax, 10% FBS at 37° C., 5% CO2. Cells were plated at 250 cells per well in white wall/clear bottom plates and incubated overnight. Twenty-four hours later, cells were dosed with Compound I or PF-07104091 as a ten-point dose curve from 300 pM to 10 mM with 10× drug stocks. Plates returned to the incubator for six additional days. On day six post dosing, plates were removed and treated with CellTiter Glo reagent following manufacturer's specifications. Plates incubated 45 minutes at room temperature and run on a Biotek luminometer with 1 sec/well detection. Results were converted to Microsoft Excel and transferred to GraphPad Prism for analysis. Compound I strongly inhibits the viability of SCLC model H526 cells and exceeds that of Pfizer selective CDK2 inhibitor PF-07104091, with a cell proliferation IC₅₀ value (62 nM) almost 5-fold lower than PF-07104091 (305 nM) (FIG. 22B-C).

The H526 human lung carcinoma cell line was grown in RPMI-1640/1× Glutamax/10% fetal bovine serum and was plated at 1,000 cells/well in Corning 96 well white wall/clear bottom tissue culture treated plates. Cells were incubated at 37° C. for 24 hours prior to addition of staurosporine (positive control), Compound I or PF-07104091 from 300 pM to 10 μM in triplicate for 72 hours. Plates were removed from incubator and treated with CaspaseGlo-3/7 (Promega, Inc) following manufacturer specifications. Plates were then read on a Biotech luminometer with 1 sec/well integration. Results were converted to Microsoft Excel and transferred to GraphPad Prism for Analysis (FIG. 22D). Although Compound I had a higher caspase 3/7 activation IC₅₀ value (116 nM) than the broad-spectrum, non-targeted kinase inhibitor staurosporine (6 nM), Compound I had a 3.84-fold lower IC₅₀ value than the selective CDK2 inhibitor PF-07104091 (495 nM) (FIG. 22E).

### Compound I Induces Cell Cycle and DNA Content Changes in Rb-Independent Small Cell Lung Cancer (SCLC) Model Cells

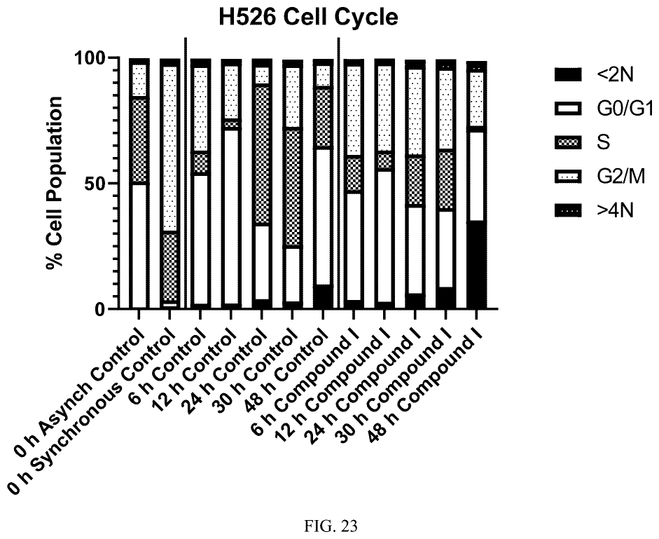

The H526 human lung carcinoma cell line was grown in RPMI-1640/1× Glutamax/10% fetal bovine serum and was plated at 200,000 cells/well in Corning 6 well tissue culture treated plates. Cells were incubated at 37° C. for 24 hours prior to addition of 50 ng/mL nocodazole to arrest the cells in G2/M. One plate was untreated for comparison. After 16 hours, the asynchronous well and one synchronized well were collected. The remaining cells were washed and half were treated with un-treated media while the remaining half were treated with media containing 300 nM Compound I. Paired plates were then collected at the indicated time points following release. Cells were incubated with 10 μM EdU for 1 hour prior to collected and were then fixed in 1% paraformaldehyde. Cells were incubated with Pacific blue azide dye as described by Thermo Scientific. Cells were then incubated with FarRed dye and RNase. Cells were analyzed on a BD Celesta and data compiled using FloJo and GraphPad (FIG. 23). Cells survived slightly better with lower nocodazole dose (50 vs 100 ng/mL) but there was still cell death observed, particularly following S phase after release. In general, H526 cells show that most cells progress from G2 to G1 following the switch from nocodazole to Compound I (FIG. 23). Cells treated with Compound I were slower to enter S phase. It is unclear whether these cells make it back to G2 or if there is a population of Compound I cells that stay arrested in G2 from the start.

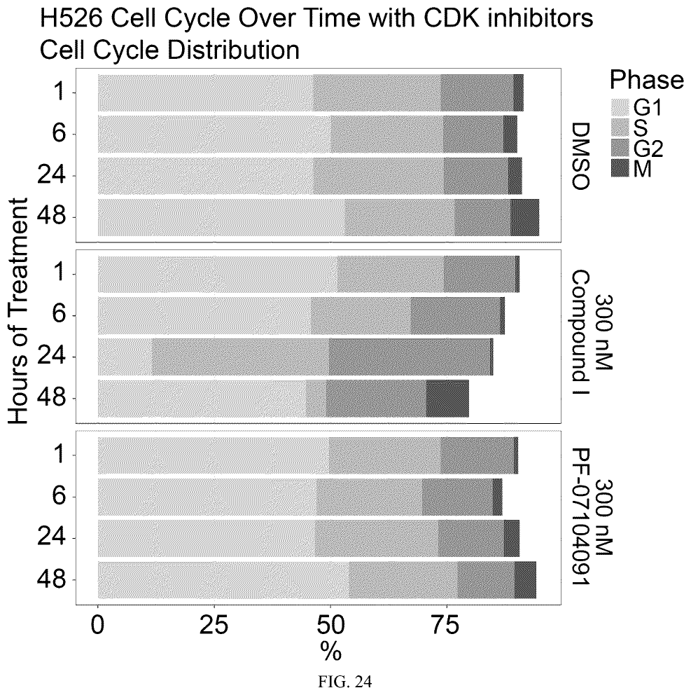

The H526 human lung carcinoma cell line was grown in RPMI-1640/1× Glutamax/10% fetal bovine serum and was plated at 200,000 cells/well in Corning 6 cm tissue culture treated plates. Cells were incubated at 37° C. for 24 hours prior to addition of 0.1% DMSO control, or 300 nM of Compound I or PF-07104091. Cells were treated for the indicated amount of time, with 10 μM EdU added for the last hour of treatment. Cells were collected and fixed in 1% paraformaldehyde. Cells were incubated with Alexa 488 azide dye as described by Thermo Scientific. Cells were then incubated with FarRed dye and RNase. Cells were analyzed on a BD Celesta and data compiled using FloJo and GraphPad (FIG. 24). Substantially fewer H526 cells treated with Compound I were in G1 phase at 24 hours compared with PF-07104091. The majority of H526 cells treated with Compound I were in either S phase or G2 phase. At 48 hours, substantially fewer H526 cells treated with Compound I were in S phase compared with H526 cells treated with PF-07104091.

The Administration of Compound I in Combination with a Chemotherapeutic Agent Induces Increased yH2AX Levels in Rb-Independent Small Cell Lung Cancer (SCLC) Model Cells Compared with Either Agent Alone

The H526 human lung carcinoma cell line was grown in RPMI-1640/1× Glutamax/10% fetal bovine serum and was plated at 200,000 cells/well in Corning 6 cm tissue culture treated plates. Cells were incubated at 37° C. for 24 hours prior to addition of the first treatment. Initial treatments were 0.1% DMSO control, 30 to 1000 nM Compound I, 300 nM cisplatin, or a combination of 300 nM Compound I and 300 nM cisplatin. After 24 hours, one well containing 300 nM Compound I, had cisplatin added to 300 nM. Cells were treated to 24 or 48 hours post-initial treatment, with 10 μM EdU added 1 hour prior to collection. Cells were collected and fixed in 1% paraformaldehyde. Cells were incubated with pacific blue azide dye as described by Thermo Scientific. Cells were then incubated with antibody against γ-H2AX at a ratio of 1:50 before incubation with FarRed dye and RNase. Cells were analyzed on a BD Celesta and data compiled using FloJo and GraphPad.

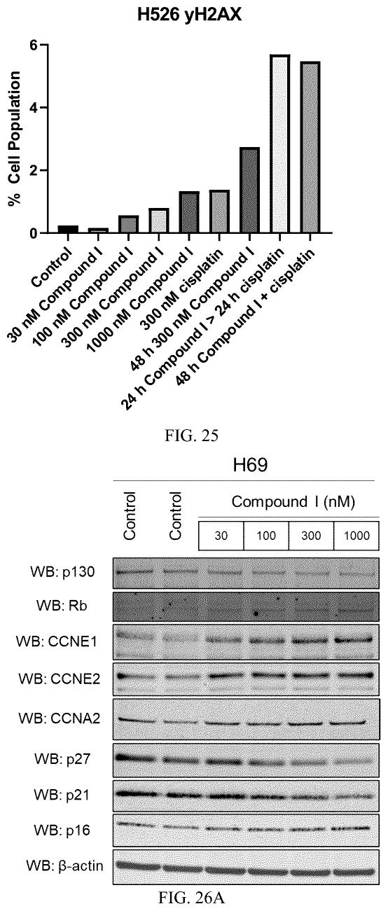

H526 cells have increased yH2AX starting at 100 nM Compound I (FIG. 25). Signal increases were observed at 300 and 1000 nM. Furthermore, signal increases were observed with time (24 v 48 h of 300 nM Compound I). Combination with cisplatin roughly double the signal. Compound I combined with cisplatin led to an increased percent of cells with γ-H2AX signal. It may be that Compound I pre-treatment prior to cisplatin treatment may be more beneficial but combination treatment from the start and lasting 48-hours also appeared to induce a similar amount of γ-H2AX-positive cells.

### Compound I Provokes DNA-Damage Response in Rb-Independent Small Cell Lung Cancer (SCLC) Model Cells

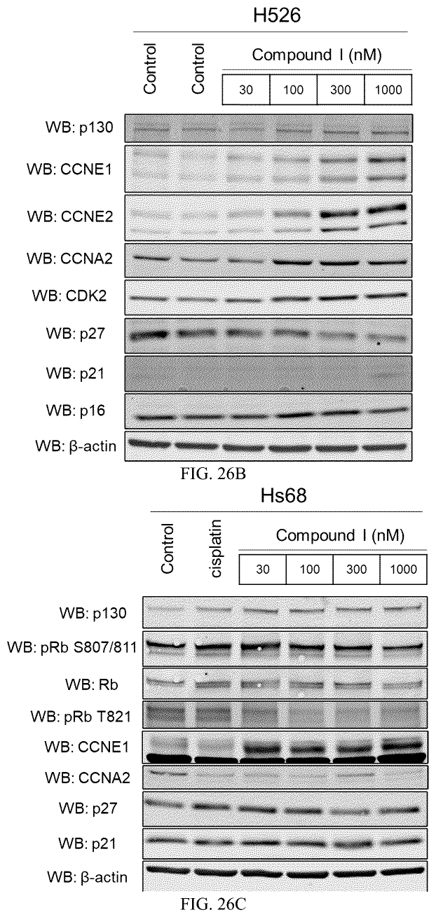

The H69 and H526 human lung carcinoma cell lines were grown in RPMI-1640/1× Glutamax/10% fetal bovine serum while the Hs68 Normal foreskin fibroblast cell line was grown in DMEM-high glucose/1× Glutamax/10% fetal bovine serum. Cells were plated at 200,000 cells/well in Corning 6 well tissue culture treated plates and were incubated at 37° C. for 24 hours prior to addition of 0.1% DMSO control, 1 mM cisplatin, or the indicated concentration of Compound I. Cells were treated for 24 hours. Cells were collected by washing each well twice with cold PBS, then scraping cells in the presence of 150 μL cold RIPA with protease and phosphatase inhibitors. Lysates were clarified by centrifugation at 14,000×g for 10 min at 4° C. and supernatants transferred to clean tubes. A BCA assay was performed to determine the protein concentration of each lysate. A total of 30 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBS-T, membranes were incubated for 45 minutes in LI-COR secondary antibodies. Following 2 washes in TBS-T, and one TBS wash, membranes were scanned using a LI-COR OdysseyCLx. Image studio was used for image analysis. An increase of cyclin E (CCNE1, CCNE2) amplification was observed in SCLC model H69 cells (FIG. 26A) and H526 cells (FIG. 26B) as well as healthy Hs68 fibroblast cells (FIG. 26C) administered Compound I. No overt DNA damage marker changes were observed in H69 and H526 cells as a result of increased Compound I doses, having similar expression patterns of cell cycle inhibitor proteins p130, p27, and p21 with that of fibroblast Hs68 cells.

### Compound I Induces Increased Cell Death in Rb-Independent Small Cell Lung Cancer (SCLC) Model Cells Relative to CDK2 Inhibitor PF-07104091

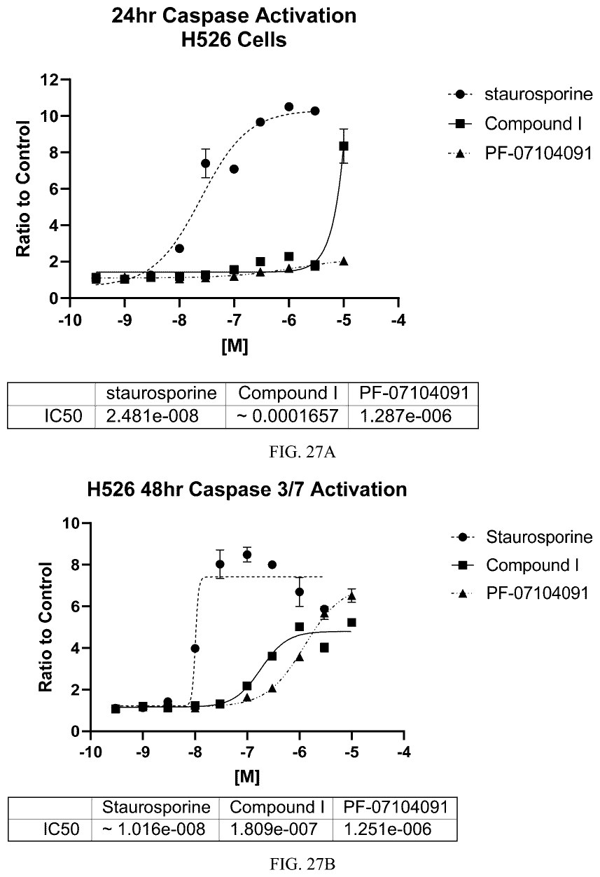

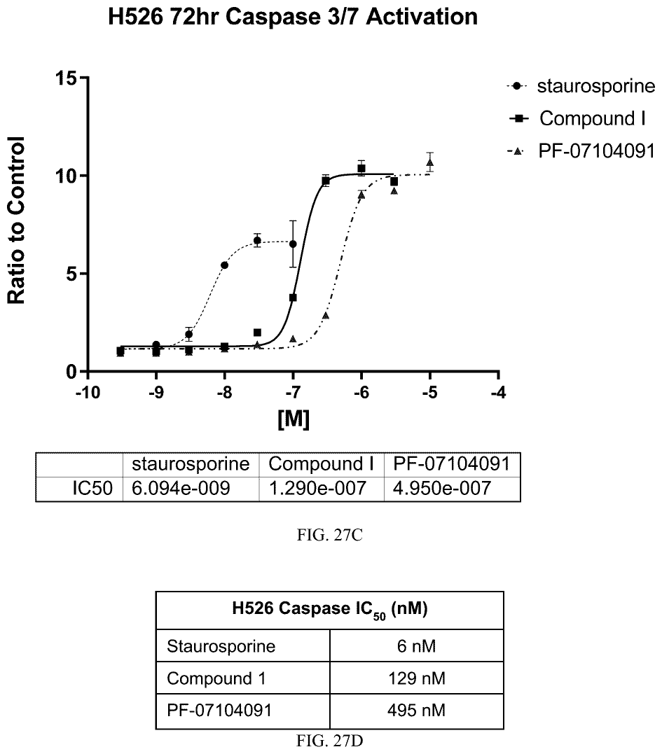

The H526 human lung carcinoma cell line was grown in RPMI-1640/1× Glutamax/10% fetal bovine serum and was plated at 1,000 cells/well in Corning 96 well white wall/clear bottom tissue culture treated plates. Cells were incubated at 37° C. for 24 hours prior to addition of staurosporine (positive control), Compound I or PF-07104091 from 300 pM to 10 μM in triplicate for 24, 48 or 72 hours. Plates were removed from the incubator at each specified timepoint and treated with CaspaseGlo-3/7 (Promega, Inc) following manufacturer specifications. Plates were then read on Biotech luminometer with 1 sec/well integration. Results were converted to Microsoft Excel and transferred to GraphPad Prism for Analysis (FIG. 27A-C). The broad-spectrum, non-targeted kinase inhibitor staurosporine induced robust caspase 3/7 activation (a marker of cell death) in H526 cells at 24 hours post treatment initiation, the first time point tested (FIG. 27A). At 24 hours, no appreciable caspase 3/7 activation was observed in cells administered either Compound I or PF-07104091. At 48 hours, however, both Compound I and PF-07104091 began to induce caspase 3/7 activation (FIG. 27B), with Compound I exhibiting a nearly 7-fold lower IC₅₀ value (181 nM) than PF-07104091 (1251 nM). By 72 hours, Compound I retained a nearly 4-fold lower IC₅₀ value (129 nM) than that of PF-07104091 (495 nM) (FIG. 27D).

Administration of a Combination of Compound I and Cisplatin Enhances yH2AX Levels in Small Cell Lung Cancer (SCLC) Model Cells

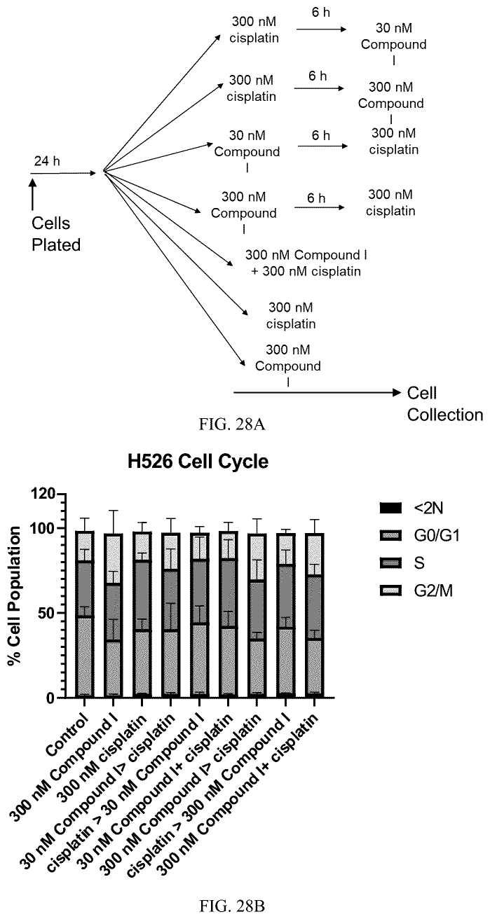

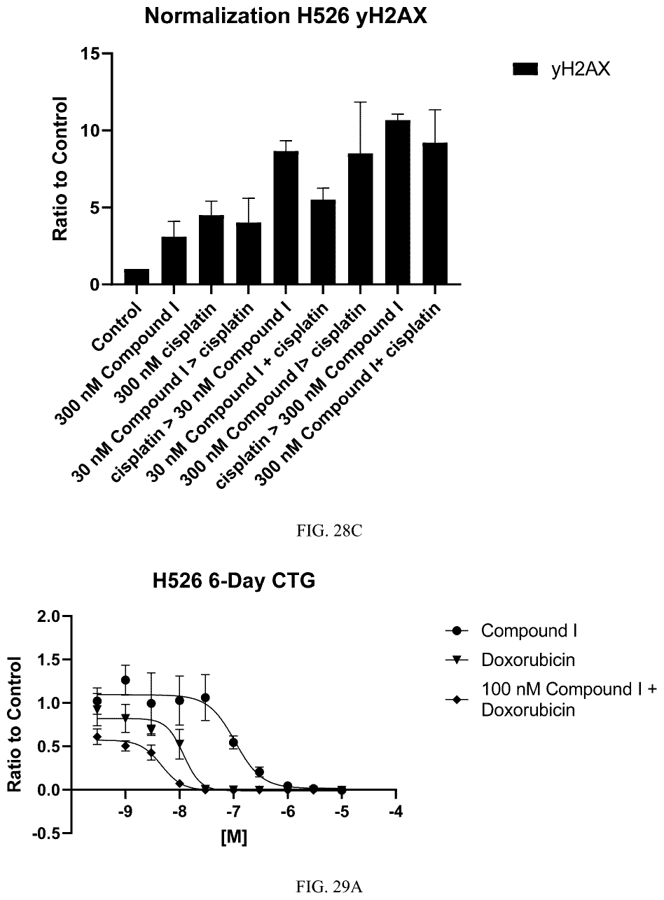

The H526 human lung carcinoma cell line was grown in RPMI-1640/1× Glutamax/10% fetal bovine serum and was plated at 200,000 cells/well in Corning 6 cm tissue culture treated plates. Cells were incubated at 37° C. for 24 hours prior to addition of the first treatment. Initial treatments were 0.1% DMSO control, 300 nM Compound I, 300 nM cisplatin, or a combination of 300 nM Compound I and 300 nM cisplatin. After 6 hours, one well containing Compound I, had cisplatin added to 300 nM, and one well containing cisplatin had Compound I added to 300 nM. Cells were treated to 24 hours post-initial treatment, with 10 μM EdU added at hour 23 (FIG. 28A). Cells were collected and fixed in 1% paraformaldehyde. Cells were incubated with pacific blue azide dye as described by Thermo Scientific. Cells were then incubated with antibody against γ-H2AX at a ratio of 1:50 before incubation with FarRed dye and RNase. Cells were analyzed on a BD Celesta and data compiled using FloJo and GraphPad.

An increase of cells in G2/M phase in cells administered 300 nM Compound I compared to control cells (FIG. 28B). Both 300 nM Compound I and Cisplatin induced similar levels of γ-H2AX at 24 hours (FIG. 28B). While 30 nM Compound I pre-treatment may have enhanced Cisplatin effects, post treatment of 30 nM Compound I was much more effective. At 300 nM Compound I, pre- or post-treatment was equally effective and induced a large amount of γ-H2AX. Staggered treatment appears to have led to an increased percent of cells positive for γ-H2AX than a combination of Compound I and cisplatin for 24 hours.

Compound I in Combination with Chemotherapeutic Agents Inhibit Small Cell Lung Cancer (SCLC) Model H526, H69, and SHP77 Cell Lines

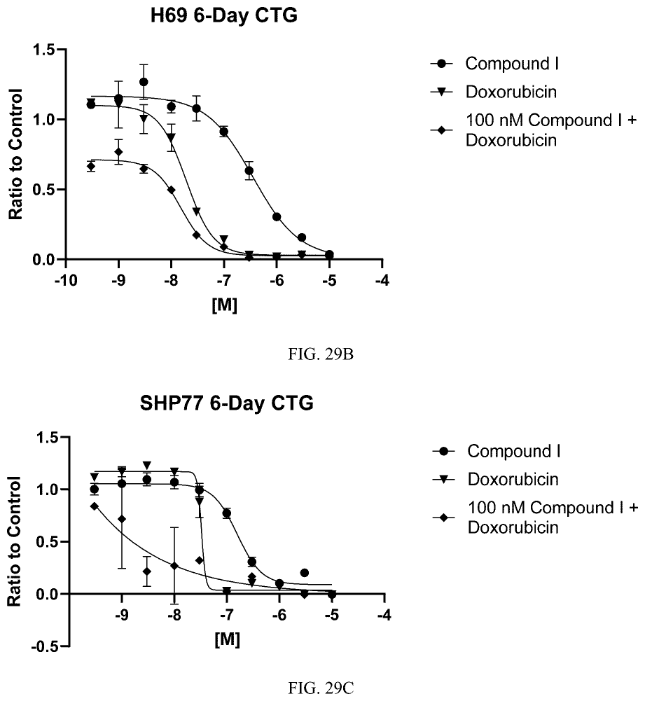

The H526, H69, and SHP77 human lung carcinoma cell lines were grown in RPMI-1640/1× Glutamax/10% fetal bovine serum. were plated at 500 cells/well in Corning 96 well white wall/clear bottom tissue culture treated plates. Cells were incubated at 37° C. for 24 hours prior to addition of Compound I dose curve, doxorubicin dose curve, or 100 nM Compound I with doxorubicin dose curve. Dose curves ranged from 300 pM to 10 μM in triplicate and treatments lasted for 6 days. Plates were removed from incubator at each specified timepoint and treated with CellTiter-Glo (Promega, Inc) following manufacturer specifications. Plates were then read on CLARIOstar luminometer with 1 sec/well integration. Results were converted to Microsoft Excel and transferred to Graphpad Prism for Analysis (FIG. 29A-C). While the administration of doxorubicin exhibited better inhibition of SCLC model H526 cells (FIG. 29A), H69 cells (FIG. 29B), and SHP77 cells (FIG. 29C) compared with Compound I alone, the combined administration of doxorubicin and Compound I led to the lowest cellular proliferation IC₅₀ values.

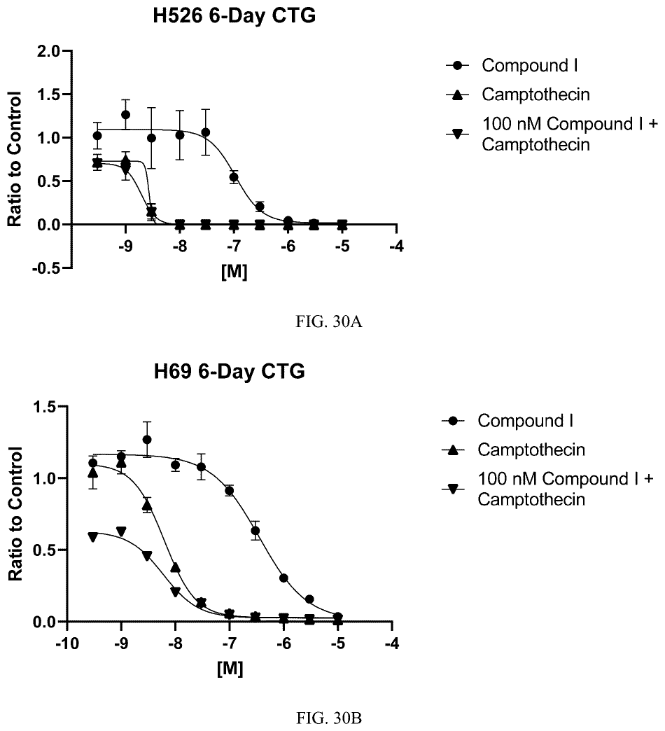

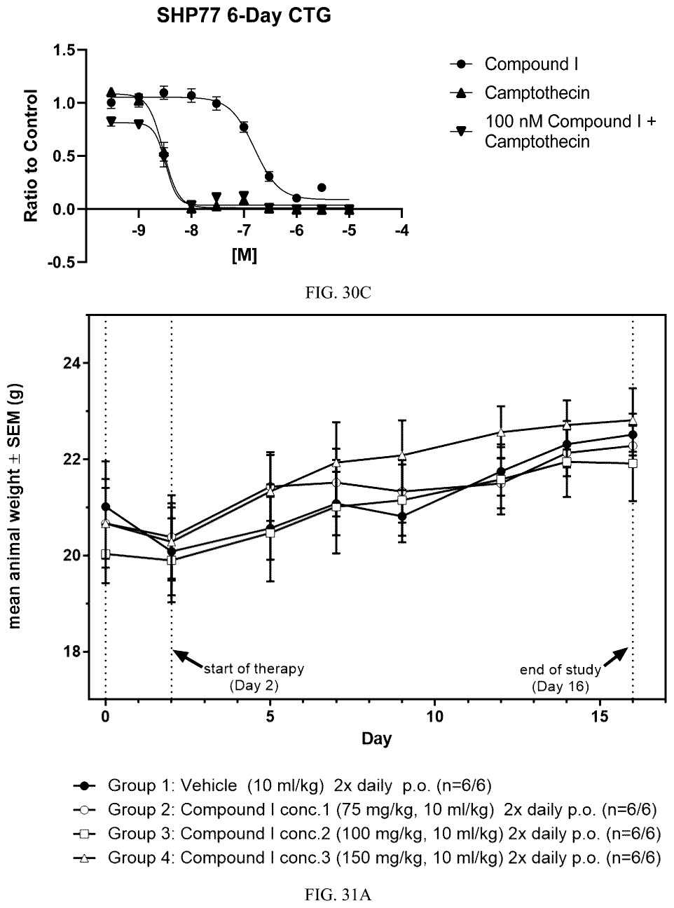

The H526, H69, and SHP77 human lung carcinoma cell lines were grown in RPMI-1640/1× Glutamax/10% fetal bovine serum. were plated at 500 cells/well in Corning 96 well white wall/clear bottom tissue culture treated plates. Cells were incubated at 37° C. for 24 hours prior to addition of Compound I dose curve, camptothecin dose curve, or 100 nM Compound I with camptothecin dose curve. Dose curves ranged from 300 pM to 10 μM in triplicate and treatments lasted for 6 days. Plates were removed from incubator at each specified timepoint and treated with CellTiter-Glo (Promega, Inc) following manufacturer specifications. Plates were then read on CLARIOstar luminometer with 1 sec/well integration. Results were converted to Microsoft Excel and transferred to GraphPad Prism for Analysis. The administration of camptothecin led to lower cellular proliferation IC₅₀ values in all SCLC model cells tested (FIG. 30A-C). H526 cells administered camptothecin alone was comparable with Compound I in combination with camptothecin (FIG. 30A). In H69 cells, however, the combination of Compound I and camptothecin led to a lower IC₅₀ value compared with either camptothecin or Compound I administered alone (FIG. 30B). SHP77 cells administered either camptothecin or Compound I alone was comparable with Compound I in combination with camptothecin (FIG. 30C).

### Example 6. Compound I Provides Anti-Tumor Potency in an In Vivo Tumor Cell-Loaded Hollow Fiber Mouse Model In Vivo

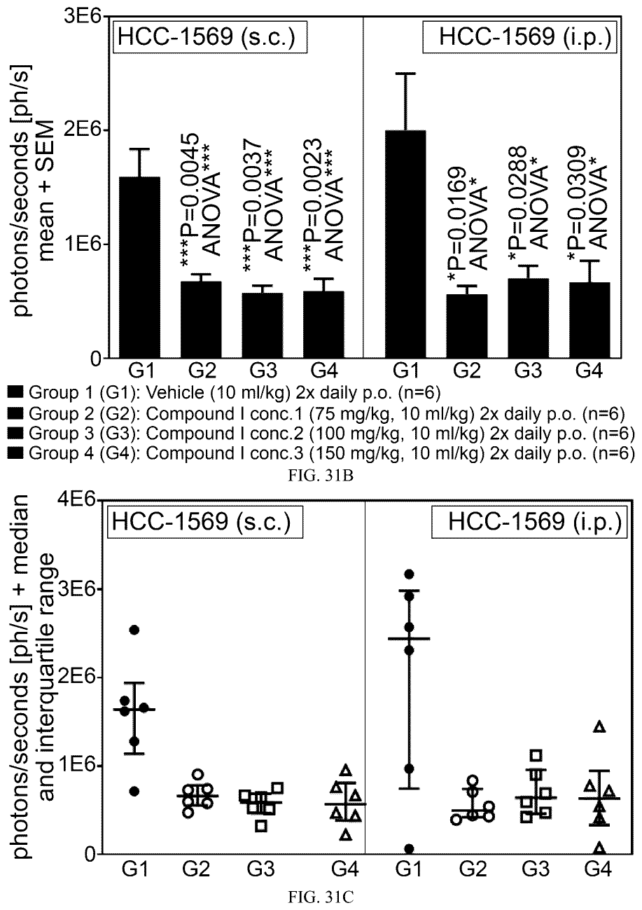

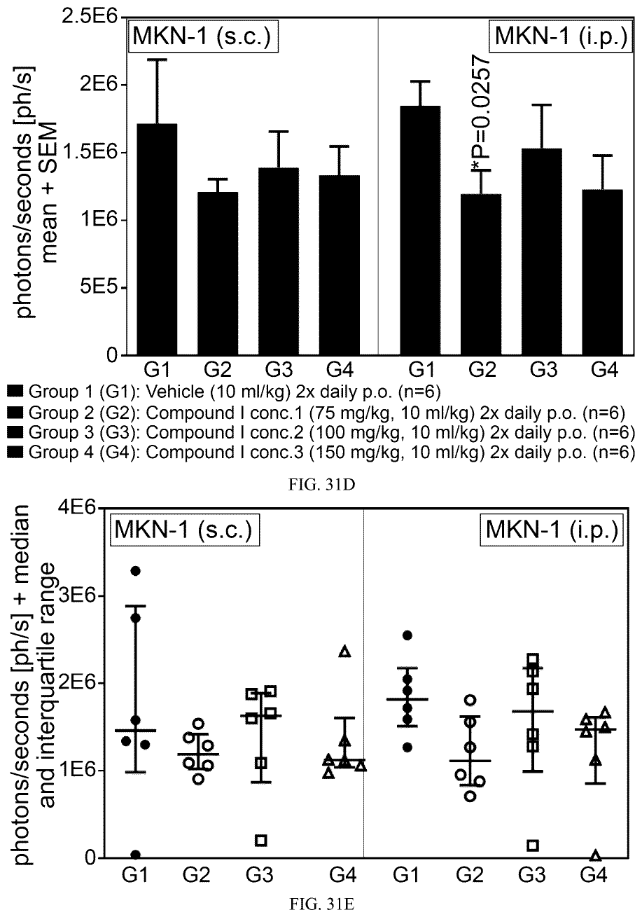

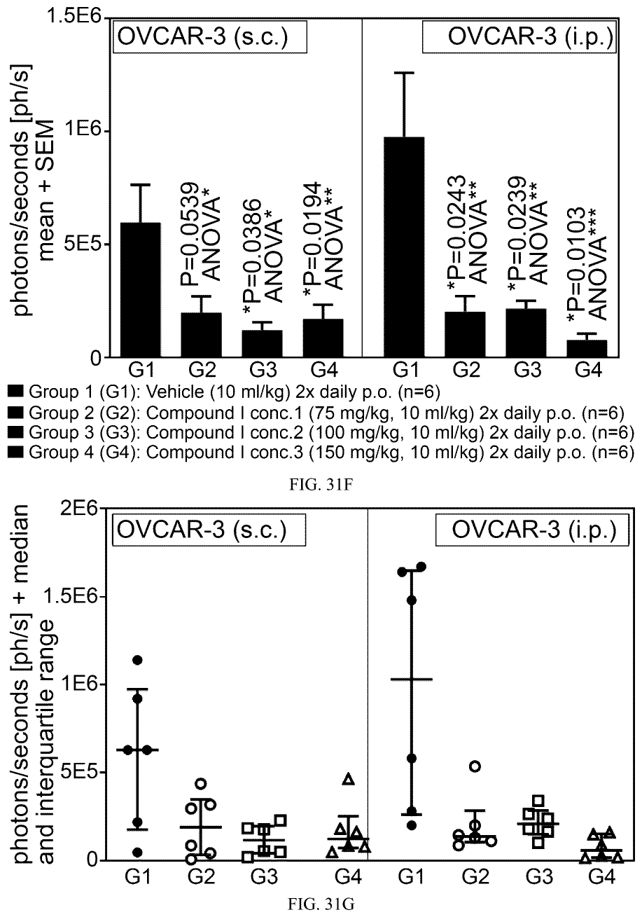

The mouse hollow fiber assay was conducted using three tumor cell lines, OVCAR-3 (ovarian), MKN-1 (gastric), and HCC-1569 (breast), 1e7 OVCAR-3, 5e6 MKN-1, and 1e7 HCC-1569 cells were loaded into Hollow Fibers, placed in cell culture dishes containing RPMI-1640 with 20% FCS and 1% penicillin/streptomycin, and were equilibrated in an incubator at 37 C, 5% CO2 overnight. On Day 0, three fibers were implanted in two different compartments: subcutaneous and intraperitoneal of twenty-four female NMRI nude mice ages 4-5 weeks. Mice were treated with Compound I on day 2 twice daily at three concentrations: 75, 100, 150 mg/kg by oral gavage. Female NMRI nude mice were implanted both subcutaneously and intraperitoneally with HCC-1569, MKN1, and OVCAR3 tumor cell-loaded Hollow Fibers on Day 0. The number of animals alive on Day 0 (implantation) and on Day 16 (necropsy) in each group is shown in parentheses in the legend. No animal weight changes were observed during the regardless of increasing concentrations of Compound I (FIG. 31A).

HCC-1569 cell loaded Hollow Fibers were implanted both subcutaneously and intraperitoneally into female NMRI nude mice on Day 0. Hollow Fibers were collected during necropsy on Day 16 and analyzed using a CellTiter Glo® assay. A significant reduction in breast cancer HCC-1569 tumor bioluminescence signal was observed with mice administered Compound I (FIG. 31B). Individual replicates are shown in FIG. 31C.

MKN-1 cell loaded Hollow Fibers were implanted both subcutaneously and intraperitoneally into female NMRI nude mice on Day 0. Hollow Fibers were collected during necropsy on Day 16 and analyzed using a CellTiter Glo® assay. A significant reduction in gastric cancer MKN-1 tumor bioluminescence signal was only observed in mice administered Compound I intraperitoneally at 75 mg/kg twice per day (FIG. 31D). Individual replicates are shown in FIG. 31E.

OVCAR-3 cell loaded Hollow Fibers were implanted both subcutaneously and intraperitoneally into female NMRI nude mice on Day 0. Hollow Fibers were collected during necropsy on Day 16 and analyzed using a CellTiter Glo® assay. A significant reduction in ovarian cancer OVCAR-3 tumor bioluminescence signal was observed with mice administered Compound I either subcutaneously or intraperitoneally (FIG. 31F). Individual replicates are shown in FIG. 31G.

All publications and patent applications cited in this specification are herein incorporated by reference as if each individual publication or patent application were specifically and individually indicated to be incorporated by reference.

The descriptions herein are described by way of illustration and example for purposes of clarity of understanding for embodiments only. It can be readily apparent to one of ordinary skill in the art in light of the teachings of this invention that certain changes and modifications may be made thereto without departing from the spirit or scope of the invention as defined in the appended claims.

### Example 7. Solvent Solubility Experiment for Compound I Free Base

The solubility of Compound I free base was assessed in 12 solvent systems (2-methyl-THF, 2-propanol, 2-propanol:water (80:20% v/v), acetone, anisole, dimethylsulfoxide, ethanol, ethyl acetate, methylethyl ketone, N,N-dimethylacetamide, THF, and toluene). The following procedure was carried out: 25, 50 and 100 μL aliquots of the appropriate solvent system was added to 20 mg of free base. The experiments were checked for dissolution. Where dissolution was not observed, the experiments were stirred at 40° C. to check for dissolution at elevated temperature. Aliquots of solvent were added until dissolution was observed, or 2 mL of total solvent was added. Any clear solutions were uncapped and allowed to evaporate at ambient temperature (ca. 20° C.) and pressure. Any slurries were isolated by centrifugation and the solids were analyzed by XRPD.

The results for the approximate solubility experiments of free base are presented in Table 1.

The following observations and results were obtained from the approximate solubility experiments. Free base had a low solubility (<10 mg/mL) in the majority of the solvent systems investigated. Free base had good solubility in dimethylsulfoxide (DMSO) (88 mg/mL) and N,N-dimethylacetamide (DMA) (22 mg/mL).

TABLE 1
Observations and Results of Approximate Solubility of Free Base

| Exper- | | | Solubility | |
|---|---|---|---|---|
| iment | Solvent | Observation | (mg/mL) | XRPD |
| 1 | 2-Methyl-THF | White slurry | <10 | FB 1 |
| 2 | 2-Propanol | White slurry | <10 | FB 1 |
| 3 | 2-Propanol:Water | White slurry | <10 | FB 2 |
| | (80:20 % v/v) | | | |
| 4 | Acetone | White slurry | <10 | FB 3 |
| 5 | Anisole | White slurry | <10 | FB 1 |
| 6 | Dimethylsulfoxide | Pale yellow | 88 | N/A |
| | | solution | | |
| 7 | Ethanol | White slurry | <10 | FB 1 |
| 8 | Ethyl Acetate | White slurry | <10 | FB 1 |
| 9 | Methylethyl Ketone | White slurry | <10 | poorly |
| | | | | crystalline |
| 10 | N,N-Dimethylacetamide | Pale yellow | 22 | N/A |
| | | solution | | |
| 11 | Tetrahydrofuran | White slurry | <10 | FB 1 |
| 12 | Toluene | White slurry | <10 | FB 1 |

Note:
FB 1: Free Base Pattern 1;
FB 2: Free Base Pattern 2;
FB 3: Free Base Pattern 3

### Example 8. Primary Salt Experiments

The primary salt experiment of Compound I free base was carried out in six solvent systems, such as 2-propanol:water (80:20% v/v), acetone, acetonitrile, ethanol, ethyl acetate, and tetrahydrofuran (THF), using eight counterions formed from the following acids: sulfuric acid, methanesulfonic acid, maleic acid, phosphoric acid, L-(+)-tartaric acid, citric acid, hydrobromic acid, and benzenesulfonic acid. The following procedure was carried out for the primary salt experiment of free base: 1 mL (for liquid counterions) or 0.5 mL (for solid counterions) of solvent was added to ca. 20 mg of free base and stirred at 25° C.; then, 1.05 eq. of the appropriate counterion was added to the experiments. Where solid counterions were used, the vial which contained the counterion was washed into the vial containing free base using 0.5 mL of the appropriate solvent.

The experiments were thermally cycled as follows:
Heat from 25 to 40° C. at a rate of 0.1° C./min;
Hold for 1 h at 40° C.;
Cool to 5° C. at a rate of 0.1° C./min;
Hold for 1 h at 5° C.;
Heat to 40° C. at a rate of 0.1° C./min;
Cycle between 4° and 5° C.
After 2-3 days, the slurries were isolated by centrifugation.

XRPD analysis was carried out on any isolated solids or gums as described in Example 10 below. The XRPD plate, and the isolated solids, were dried at 40° C. for ca. 24 hours and re-analyzed by XRPD. The XRPD plate was further dried at 40° C./75% RH for ca. 24 hours and XRPD analysis was repeated.

### Sulfuric Acid Salt

The following results were obtained for free base with sulfuric acid (Table 2). No dissolution of free base was observed upon addition of acid. Slurries were observed for all solvent systems. Faint yellow slurries were obtained in the 2-propanol:water, acetone, ethyl acetate and tetrahydrofuran solvent systems. White slurries were obtained in the acetonitrile and ethanol solvent systems. Slurries were observed in all solvent systems after thermal cycling for two days. A color change from faint yellow to white was observed for the material in the ethyl acetate solvent system. A unique XRPD pattern was observed for the material isolated from each solvent system. No changes were observed in the materials upon drying.

TABLE 2
Observations and Results of Free Base with Sulfuric Acid

| | | | XRPD Pattern | | |
|---|---|---|---|---|---|
| | Observation | | | | 40° C./75% |
| Solvent | Initial | 2 Day | Wet | Dry | RH |
| 2-Propanol:Water | FYS | OWS | 1 | 1 | 1 |
| 80:20 % v/v | | | | | |
| Acetone | FYS | FYS | 2 | 2 | n/a |
| Acetonitrile | WS | WS | 3 | 3 | n/a |
| Ethanol | WS | WS | 4 | 4 | 4 |
| Ethyl Acetate | FYS | WS | 5 | 5 | n/a |
| Tetrahydrofuran | FYS | FYS | 6 | 6 | 6 |

Notes:
FYS: Faint Yellow Slurry;
WS: White Slurry;
OWS: Off White Slurry;
n/a: not available

In an attempt to produce more Pattern 5 material, the experiment was repeated. The observations and results of the repeated reaction of free base with sulfuric acid are presented in Table 3.

TABLE 3
Observations and Results for Free
Base with Sulfuric Acid (Repeat)

| | | | XRPD Pattern | | |
|---|---|---|---|---|---|
| | Observation | | | | 40° C./75% |
| Solvent | Initial | 2 Day | Wet | Dry | RH |
| Ethyl Acetate | WS | WS | 7 | 7 | 7 |

Notes:
WS: White Slurry

The following results were obtained. No dissolution of free base was observed upon addition of acid. A white slurry was obtained. No color change was observed after thermal cycling for two days and a white slurry was obtained. A new diffraction pattern was observed for the material isolated from the repeat reaction which showed slight differences to that of the original material. No subsequent changes in the material were observed upon drying.

### Methanesulfonic Acid Salt

The following results were obtained for methanesulfonic acid (Table 4). No dissolution of free base was observed upon addition of acid. Faint yellow slurries were observed for all solvent systems. Slurries were observed for all solvent systems after thermal cycling for two days. A color change from faint yellow to white was observed in the 2-propanol:water, acetonitrile, ethanol, ethyl acetate and tetrahydrofuran solvent systems. No color change was observed for the acetone solvent system. Four unique diffraction patterns were initially identified. No changes were observed in the diffraction pattern of the material after drying at 40° C. under vacuum. Drying the materials at 40° C./75% RH resulted in conversion of all diffraction patterns to Pattern 1.

TABLE 4
Observations and Results with Methanesulfonic Acid

| | | | XRPD Pattern | | |
|---|---|---|---|---|---|
| | Observation | | | | 40° C./75% |
| Solvent | Initial | 2 Day | Wet | Dry | RH |
| 2-Propanol:Water | FYS | WS | 1 | 1 | 1 |
| 80:20 % v/v | | | | | |
| Acetone | FYS | FYS | 2 | 2 | 1 |
| Acetonitrile | FYS | WS | 3 | 3 | 1 PC |
| Ethanol | FYS | WS | 4 | 4 | 1 |
| Ethyl Acetate | FYS | WS | 1+ | 1+ | 1 |
| Tetrahydrofuran | FYS | WS | 1+ | 1+ | 1 |

Notes:
FYS: Faint Yellow Slurry;
WS: White Slurry;
+Additional peaks;
PC: Poorly Crystalline

Patterns 2, 3 and 4 converted to Pattern 1 after storage at 40° C./75% RH.

### Maleic Acid Salt

The following results were obtained with maleic acid (Table 5). No dissolution of free base was observed upon addition of acid. A faint yellow or yellow slurry was obtained for each solvent system. Slurries were obtained from all solvent systems after thermally cycling for two days. A color change from faint yellow to white was observed for the material in the acetone solvent system. Five unique diffraction patterns were initially obtained. No changes were observed in the diffraction patterns after drying at 40° C. under vacuum. Storage of the samples at 40° C./75% RH resulted in a change for Pattern 4 and Pattern 5 materials giving Pattern 6 and Pattern 1* respectively. Additional peaks were also observed for Pattern 3 material. A loss of crystallinity was observed for Pattern 2 material after storage at 40° C./75% RH.

TABLE 5
Observations and Results with Maleic Acid

| | | | XRPD Pattern | | |
|---|---|---|---|---|---|
| | Observation | | | | 40° C./75% |
| Solvent | Initial | 2 Day | Wet | Dry | RH |
| 2-Propanol:Water | YS | YS | 1 | 1 | 1 |
| 80:20 % v/v | | | | | |
| Acetone | FYS | WS | 2 | 2 | 2 PC |
| Acetonitrile | FYS | FYS | 1 | 1 | 1 |
| Ethanol | FYS | YS | 3 | 3 | 3* |
| Ethyl Acetate | YS | YS | 4 | 4 | 6 |
| Tetrahydrofuran | YS | YS | 5 | 5 | 1* |

Notes:
FYS: Faint Yellow Slurry;
YS: Yellow Slurry;
WS: White Slurry;
PC: Poorly Crystalline

A loss of crystallinity was observed for Pattern 2 after storage at 40° C./75% RH. Pattern 4 converted to Pattern 6 and Pattern 5 converted to Pattern 1* after storage at 40° C./75% RH.

### Phosphoric Acid Salt

The following results were obtained for phosphoric acid (Table 6). No dissolution of free base was observed upon addition of acid. White slurries were observed for the 2-propanol:water, acetonitrile, ethanol and ethyl acetate solvent systems. Partial dissolution of free base was observed upon addition of acid in the acetone solvent system. The material subsequently formed a thin suspension. Slurries were obtained for all solvent systems after temperature cycling for two days. A color change from faint yellow to white was observed for the material in the ethanol solvent system after temperature cycling. Free base was recovered from the 2-propanol:water and ethanol solvent systems. Diffraction patterns were similar with only minor differences in peak positions and appearance.

TABLE 6
Observations and Results with Phosphoric Acid

| | | | XRPD Pattern | | |
|---|---|---|---|---|---|
| | Observation | | | | 40° C./75% |
| Solvent | Initial | 2 Day | Wet | Dry | RH |
| 2-Propanol:Water | WS | WS | Free | Free | Free |
| 80:20 % v/v | | | Base | Base | Base |
| Acetone | Thin YS | WS | 1 | 1 | n/a |
| Acetonitrile | WS | WS | 2 | 2 | 2 PC |
| Ethanol | WS | YS | Free | Free | Free |
| | | | Base | Base | Base |
| Ethyl Acetate | WS | YS | 3 | 3 | 5 |
| Tetrahydrofuran | FYS | YS | 4 | 4 | n/a |

Notes:
FYS: Faint Yellow Slurry;
YS: Yellow Slurry;
WS: White Slurry;
PC: Poorly Crystalline

The observations and results of the repeat reactions of free base with phosphoric acid are presented in Table 7. The following results were obtained. No dissolution of free was observed upon addition of acid. Faint yellow slurries were obtained from all solvent systems. No color change was observed for the slurries after thermal cycling for two days. Faint yellow slurries were isolated from all solvent systems. Three unique patterns were generated from the ethyl acetate and tetrahydrofuran solvent systems. The new patterns showed only minor difference to the initial patterns. Pattern 6 material was found to give amorphous material after storage at 40° C./75% RH.

TABLE 7
Observations and Results with Phosphoric Acid (Repeat)

| | | | XRPD Pattern | | |
|---|---|---|---|---|---|
| | Observation | | | | 40° C./75% |
| Solvent | Initial | 2 Day | Wet | Dry | RH |
| Acetone | FYS | FYS | 6 | 6 | Amorphous |
| Acetonitrile | FYS | FYS | 2 | 2 | 2* |
| Ethyl Acetate | FYS | FYS | 7 | 7* | 7* |
| Tetrahydrofuran | FYS | FYS | 8 | 8* | 8* |

Notes:
FYS: Faint Yellow Slurry

### (+)-L-Tartaric Acid Salt

The observations and results with (+)-L-tartaric acid are presented in Table 8. The following results were obtained. No dissolution of free base was observed upon addition of acid. White slurries were observed for all solvent systems. Slurries were observed in all solvent system after thermal cycling for two days. Two unique diffraction patterns were observed with traces of free base present in the material isolated from the acetonitrile, ethanol, ethyl acetate and tetrahydrofuran solvent systems. No changes were observed in the samples after drying at 40° C. under vacuum. Pattern 2 was found to change form giving a poorly crystalline Pattern 1 material after storage at 40° C./75% RH for ca. 24 hours. No changes were observed for the Pattern 1 material isolated from the remaining solvent systems.

TABLE 8
Observations and Results with (+)-L-Tartaric Acid

| | Observation | | XRPD Pattern | | |
|---|---|---|---|---|---|
| | | 2 | | | 40° C./ |
| Solvent | Initial | Day | Wet | Dry | 75% RH |
| 2-Propanol:Water | WS | WS | 1 | 1 | 1 |
| 80:20% v/v | | | | | |
| Acetone | WS | WS | 2 | 2 PC | 1 PC |
| Acetonitrile | WS | WS | 1+ | 1+ | 1+ |
| Ethanol | WS | WS | 1+ | 1+ | n/a |
| Ethyl Acetate | WS | WS | 1+ | 1+ | 1+ |
| Tetrahydrofuran | WS | WS | 1+ | 1+ | 1+ |

Notes:
WS: White Slurry;
PC: Poorly Crystalline;
+: Additional peaks

Traces of free base were observed in acetonitrile, ethanol, ethyl acetate and tetrahydrofuran solvent systems. Pattern 2 gave a pattern which was similar to Pattern 1 with only minor difference after storage at 40° C./75% RH. Pattern 1 material was an anhydrous mono-salt with good thermal properties.

### Citric Acid Salt

The observations and results with citric acid are presented in Table 9. The following results were obtained. No dissolution of free base was observed upon addition of acid. White slurries were obtained for all solvent systems. White slurries were obtained from all solvent system after thermal cycling for two days. Three unique diffraction patterns were initially observed. No changes were observed in the diffraction patterns upon drying at 40° C. under vacuum. Storage of Pattern 1 material at 40° C./75% RH caused a loss of crystallinity however no form change was observed. Storage of Pattern 2 material at 40° C./75% RH caused a change in form to give Pattern 4.

TABLE 9
Observations and Results with Citric Acid

| | Observation | | XRPD Pattern | | |
|---|---|---|---|---|---|
| | | 2 | | | 40° C./ |
| Solvent | Initial | Day | Wet | Dry | 75% RH |
| 2-Propanol:Water | WS | WS | Free Base | Free Base | Free Base |
| 80:20% v/v | | | | | |
| Acetone | WS | WS | 1 | 1 | 1 PC |
| Acetonitrile | WS | WS | 2 | 2 | 4 PC |
| Ethanol | WS | WS | Free Base | Free Base | Free Base |
| Ethyl Acetate | WS | WS | Free Base | Free Base | Free Base |
| Tetrahydrofuran | WS | WS | 3 | 3 | n/a |

Notes:
WS: White Slurry;
PC: Poorly Crystalline

Pattern 1 dried to a poorly crystalline material after 40° C./75% RH. Pattern 2 dried to a new poorly crystalline pattern designated Pattern 4 after 40° C./75% RH.

### Hydrobromic Acid Salt

The observations and results with hydrobromic acid are presented in Table 10. The following results were obtained. No dissolution of free base was observed upon addition of acid. Yellow slurries were obtained from all solvent systems. Yellow slurries were obtained after temperature cycling for three days. No color change was observed in any solvent system. Two unique diffraction patterns were observed with no traces of free base material. No changes were observed in the diffraction patterns of the samples after drying at 40° C. under vacuum. A loss of crystallinity was observed in the Pattern 1 material isolated from ethyl acetate after storage at 40° C./75% RH for ca. 24 hours. No changes were observed in the Pattern 1 or Pattern 2 material isolated from the remaining solvent systems.

TABLE 10
Observations and Results with Hydrobromic Acid

| | Observation | | XRPD Pattern | | |
|---|---|---|---|---|---|
| | | 2 | | | 40° C./ |
| Solvent | Initial | Day | Wet | Dry | 75% RH |
| 2-Propanol:Water | FYS | FYS | 1 | 1 | 1 |
| 80:20% v/v | | | | | |
| Acetone | YS | YS | 2 | 2 | 2 |
| Acetonitrile | FYS | FYS | 1 | 1 | 1 |
| Ethanol | FYS | FYS | 1 | 1 | 1 |
| Ethyl Acetate | FYS | FYS | 1 | 1 | PC |
| Tetrahydrofuran | FYS | FYS | 1 | 1 | 1 |

Notes:
FYS: Faint Yellow Slurry;
YS: Yellow Slurry;
PC: Poorly Crystalline

A loss of crystallinity was observed for the Pattern 1 material isolated from ethyl acetate.

### Benzene Sulfonic Acid Salt

The observations and results with benzenesulfonic acid are presented in Table 11. The following results were obtained. No dissolution of free base was observed upon addition of acid. A faint pink slurry was observed in the 2-propanol:water (80:20% v/v) solvent system. A gel was observed in the acetonitrile solvent system with a small amount of yellow precipitate. Faint yellow slurries were observed in the acetone, ethanol, ethyl acetate and tetrahydrofuran solvent systems. No changes were observed in the appearance of the samples after thermal cycling for three days. Four unique diffraction patterns were initially observed. Poorly crystalline material was isolated from the acetonitrile solvent system which precluded accurate assignment of a diffraction pattern. After drying at 40° C. under vacuum for ca. 24 hours an increase in the crystallinity was observed for the material isolated from the acetonitrile solvent system. A further increase in crystallinity was observed after storage of the material at 40° C./75% RH for ca. 24 hours however the material remained predominantly amorphous. A loss in crystallinity was observed for the Pattern 3 material isolated from the tetrahydrofuran solvent system after storage at 40° C./75% RH. The material was predominantly amorphous but was found to resemble the diffraction pattern of the poorly crystalline material isolated from the acetonitrile solvent system.

TABLE 11
Observations and Results with Benzenesulfonic Acid

| | Observation | | XRPD Pattern | | |
|---|---|---|---|---|---|
| | | 2 | | | 40° C./ |
| Solvent | Initial | Day | Wet | Dry | 75% RH |
| 2-Propanol:Water | FPS | FPS | 1 | 1 | 1 |
| 80:20% v/v | | | | | |
| Acetone | FYS | FYS | 2 | 2 | 2 PC |
| Acetonitrile | FY solids + | FY solids + | PC | PC | PC |
| | Gel | Gel | | | |
| Ethanol | FYS | FYS | 3 | 3* | PC |
| Ethyl Acetate | FYS | FYS | 4 PC | 4 PC | 4 PC |
| Tetrahydrofuran | FYS | FYS | 3 | 3* | PC |

Notes:
FPS: Faint Pink Slurry;
FYS: Faint Yellow Slurry;
FY: Faint Yellow;
PC: Poorly Crystalline

### Example 9. Hydrochloride Salt

The experiments were carried out using two equivalents of HCl, and one equivalent of HCl in ethanol.

When using two equivalents of hydrochloric acid, the following procedure was carried out. Free base (20 mg) was suspended in 1 mL of each of the six solvent systems (2-propanol:water (80:20% v/v), acetone, acetonitrile, ethanol, ethyl acetate, and THF). Then, 2 equivalents of hydrochloric acid were added neat to each sample and initial observations were recorded. The suspensions were thermally cycled for three days according to the following cycle:
Heat to 40° C. at a rate of 0.1° C./min;
Hold at 40° C. for one hour;
Cool to 5° C. at a rate of 0.1° C./min;
Hold at 5° C. for one hour.

After three days, observations were recorded and solids were isolated by centrifugation. The wet solids were analyzed by XRPD. Solids were dried at 40° C. under vacuum for 72 hours and re-analyzed by XRPD. Solids were dried at 40° C./75% RH for 18 hours and re-analyzed by XRPD.

The observations and results of the experiment with two equivalents of hydrochloric acid are presented in Table 12. No dissolution of free base was observed upon addition of acid. A faint yellow slurry was obtained in all solvent systems. Thermal cycling for three days led to a color change in all solvent systems with a pink slurry being observed in the 2-propanol/water solvent system. A yellow slurry was observed from acetone while a white slurry was observed in the acetonitrile, ethanol, ethyl acetate and tetrahydrofuran solvent systems. Hydrochloride Pattern 3 was observed from the acetone solvent system; however, acetone was suspected to be incompatible with free base. Pattern 2 material was observed from the ethanol solvent system. The diffraction pattern for the material isolated from the 2-propanol:water, acetonitrile, ethyl acetate, and tetrahydrofuran solvent systems indicated HCl Pattern 1 of a mono-salt. No changes were observed in the material upon drying or after storage at 40° C./75% RH.

TABLE 12
Observations and Results with Two
Equivalents of Hydrochloric Acid

| | Observation | | XRPD Pattern | | |
|---|---|---|---|---|---|
| | | 3 | | | 40° C./ |
| Solvent | Initial | Day | Wet | Dry | 75% RH |
| 2-Propanol:Water | FYS | PS | 1 | 1 | 1 |
| 80:20% v/v | | | | | |
| Acetone | FYS | YS | 3 | 3 | 3 |
| Acetonitrile | FYS | WS | 1 | 1 | 1 |
| Ethanol | FYS | WS | 2 | 2 | 2 |
| Ethyl Acetate | FYS | WS | 1 PC | 1 PC | 1 |
| Tetrahydrofuran | FYS | WS | 1 | 1 | 1 |

Notes:
PS: Pink Slurry;
FYS: Faint Yellow Slurry;
WS: White Slurry;
PC: Poorly Crystalline

When HCl salt was prepared from the free base (20 mg) using one equivalent of HCl in ethanol (1 mL), the following procedure was carried out. The suspension of hydrochloride salt was thermally cycled for three days according to the following cycle:
Heat to 40° C. at a rate of 0.1° C./min;
Hold at 40° C. for one hour;
Cool to 5° C. at a rate of 0.1° C./min;
Hold at 5° C. for one hour.

After three days, observations were recorded and solids were isolated by centrifugation. Wet solids were analyzed by XRPD.

The observations and results of free base with one equivalent of hydrochloric acid using only ethanol as a solvent are presented in Table 13.

The following results were obtained. No dissolution of free base was observed upon addition of acid. A faint yellow slurry was obtained. Thermal cycling for three days led to a color change in slurry for faint yellow to white. No unique diffraction pattern was observed. The material was to be Pattern 2, a match to the material isolated from the ethanol solvent system when two equivalents of HCl were employed. No form changes were observed upon drying or after storage at 40° C./75% RH.

TABLE 13
Observations and Results with One Equivalent
of Hydrochloric Acid in Ethanol

| | |
|---|---|
| Observation | XRPD Pattern |

| | | 3 | | | 40° C./ |
|---|---|---|---|---|---|
| Solvent | Initial | Day | Wet | Dry | 75% RH |
| Ethanol | FYS | WS | 2 | 2 | 2 |

Notes:
FYS: Faint Yellow Slurry;
WS: White Slurry

### Example 10. X-Ray Powder Diffraction (XRPD)

XRPD analysis was carried out on a PANalytical X'pert pro with PIXcel detector (128 channels), scanning the samples between 3 and 35° 20. The material was gently ground to release any agglomerates and loaded onto a multi-well plate with Mylar polymer film to support the sample. The multi-well plate was then placed into the diffractometer and analyzed using Cu K radiation (α1 λ=1.54060 Å; α2=1.54443 Å; β=1.39225 Å; α1:α2 ratio=0.5) running in transmission mode (step size 0.0130° 20, step time 18.87s) using 40 kV/40 mA generator settings. Data were visualized and images generated using the HighScore Plus 4.9 desktop application (PANalytical, 2017).

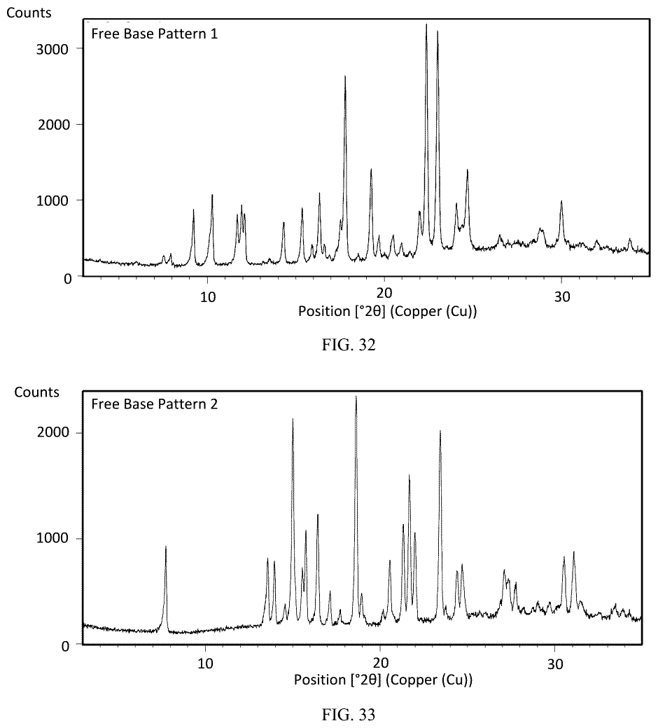

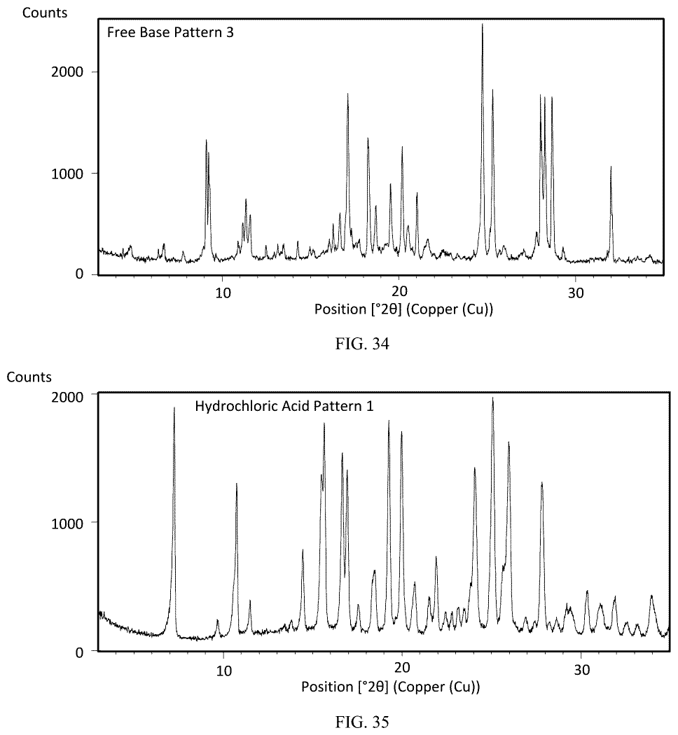

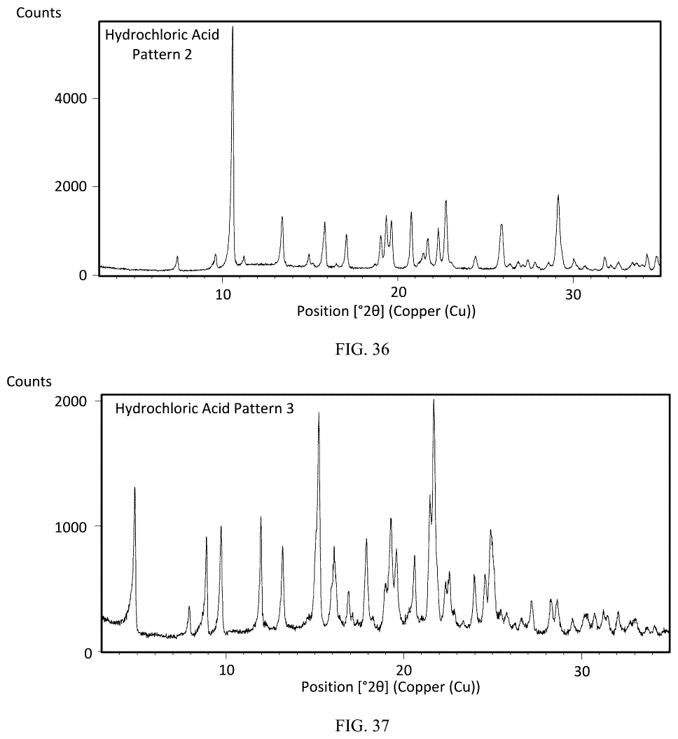

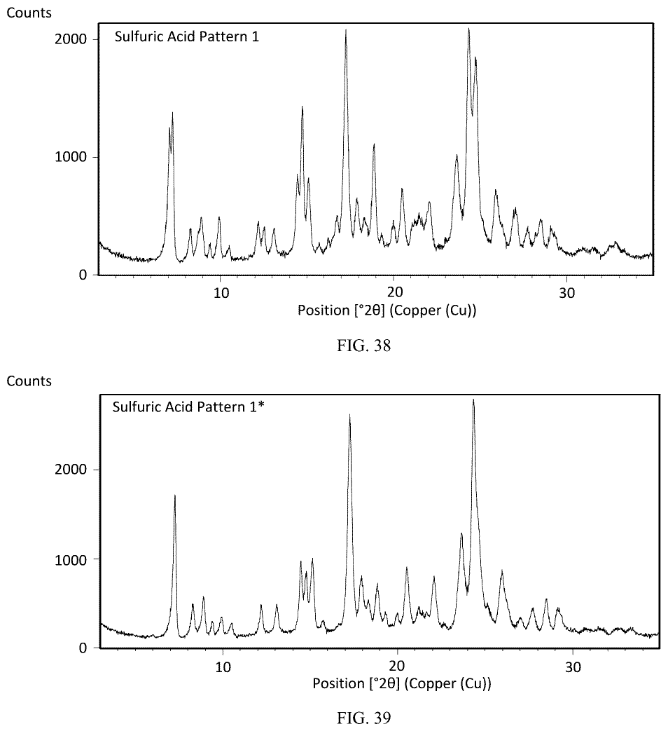

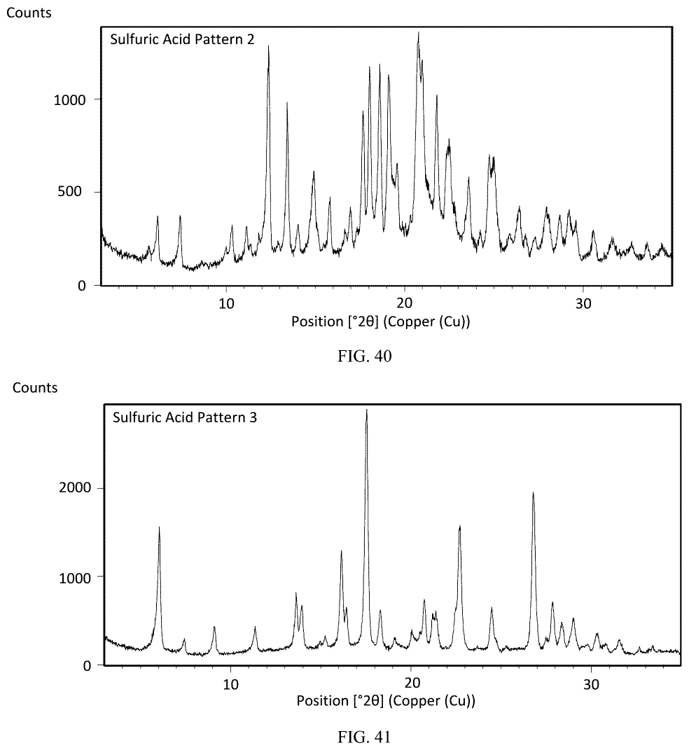

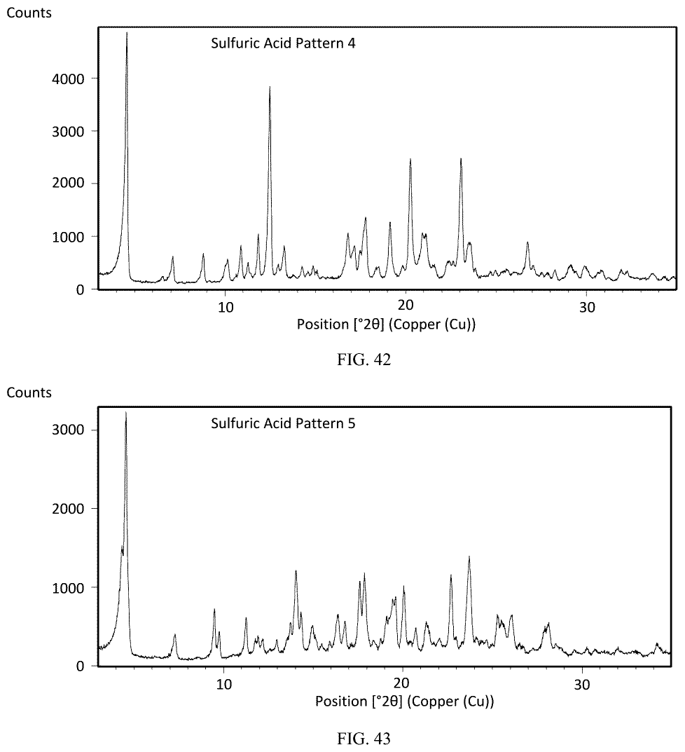

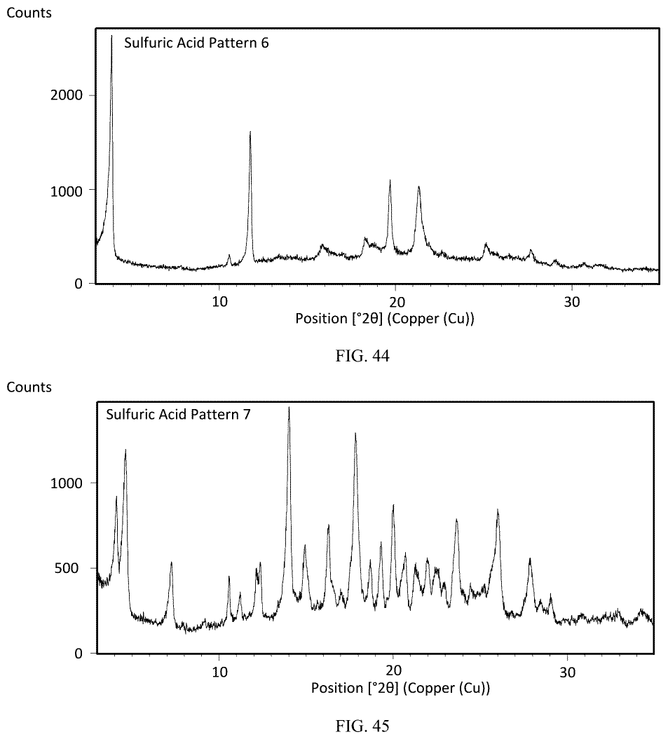

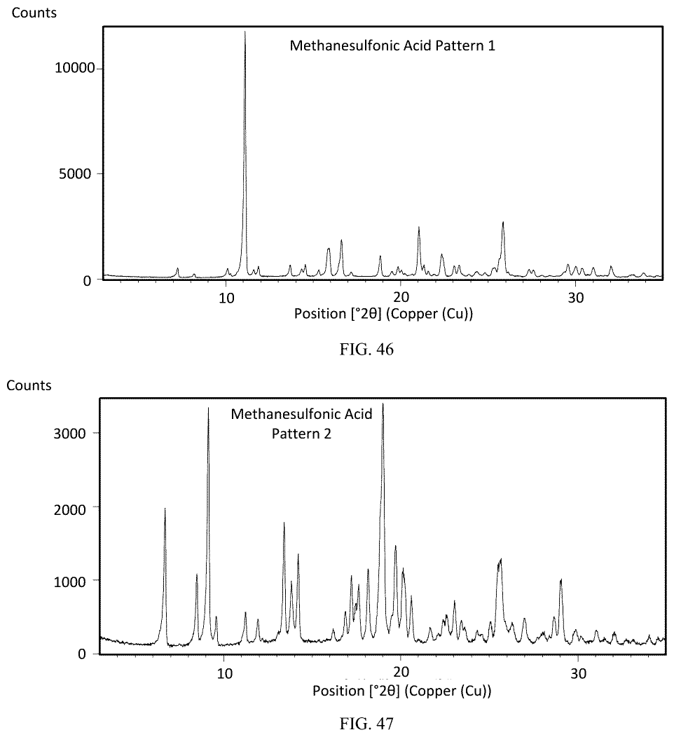

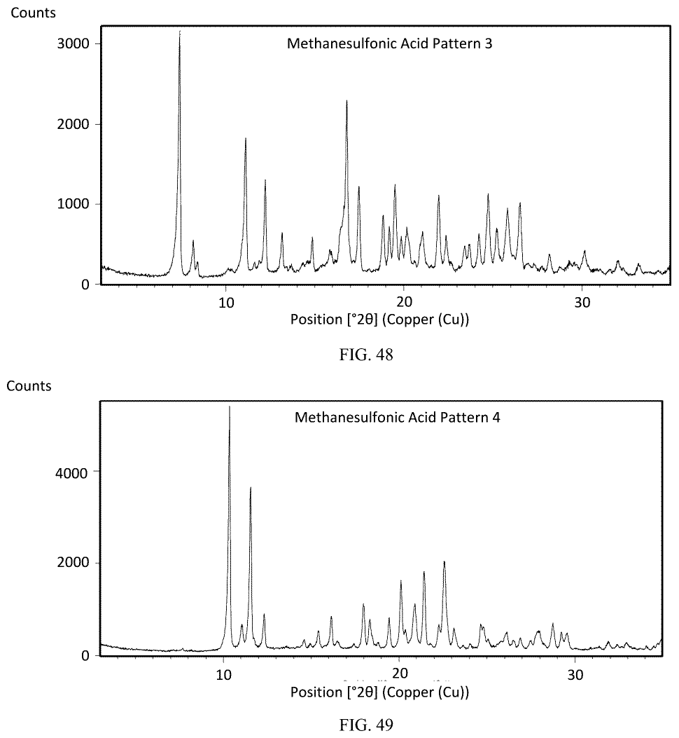

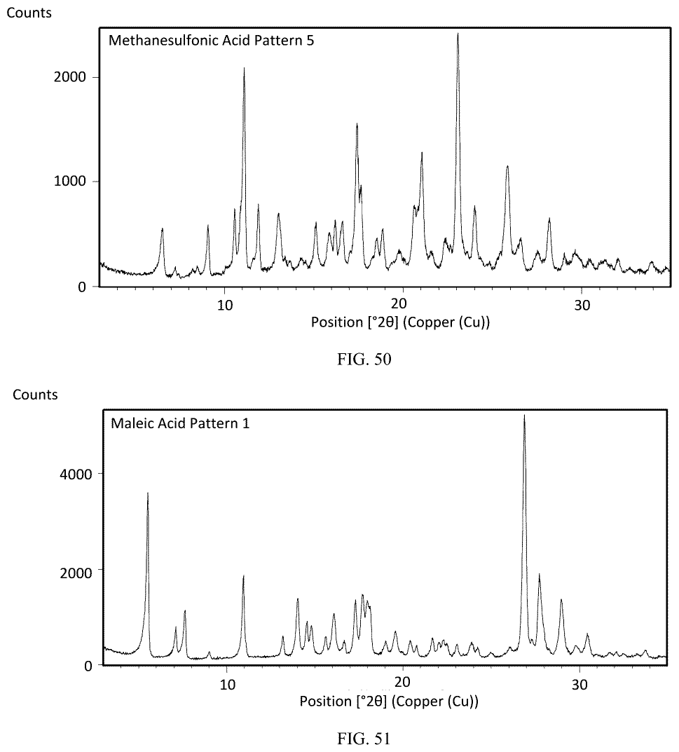

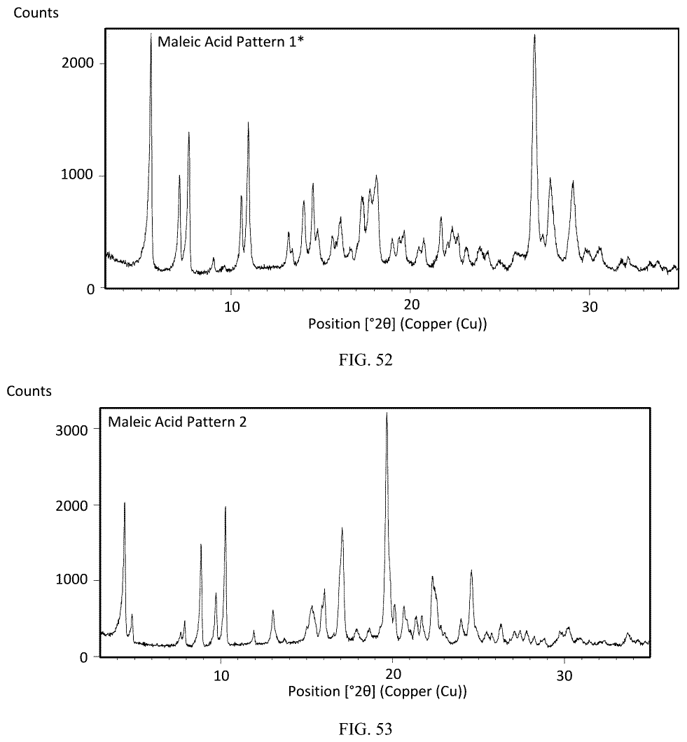

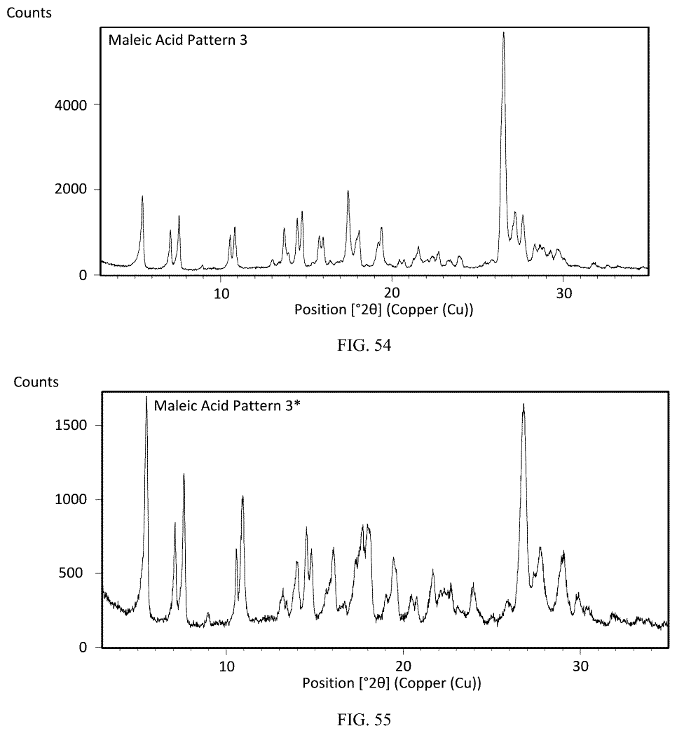

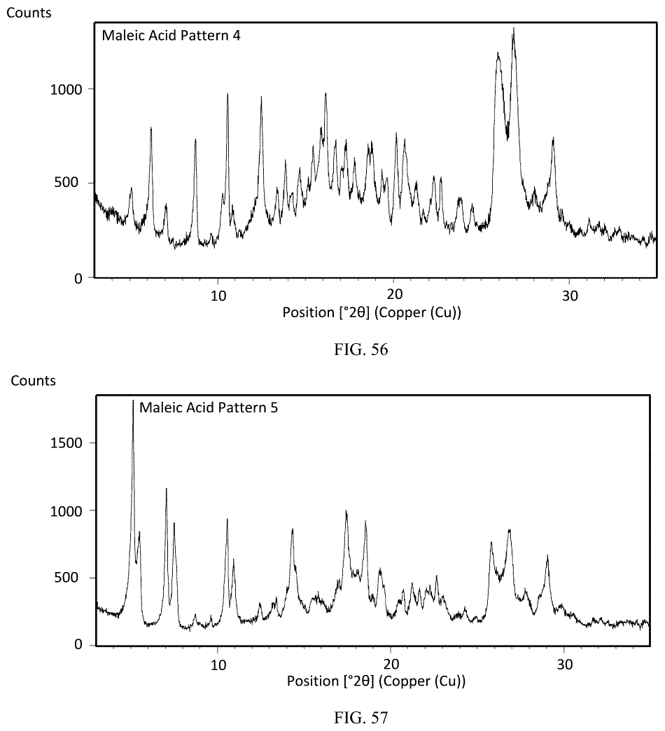

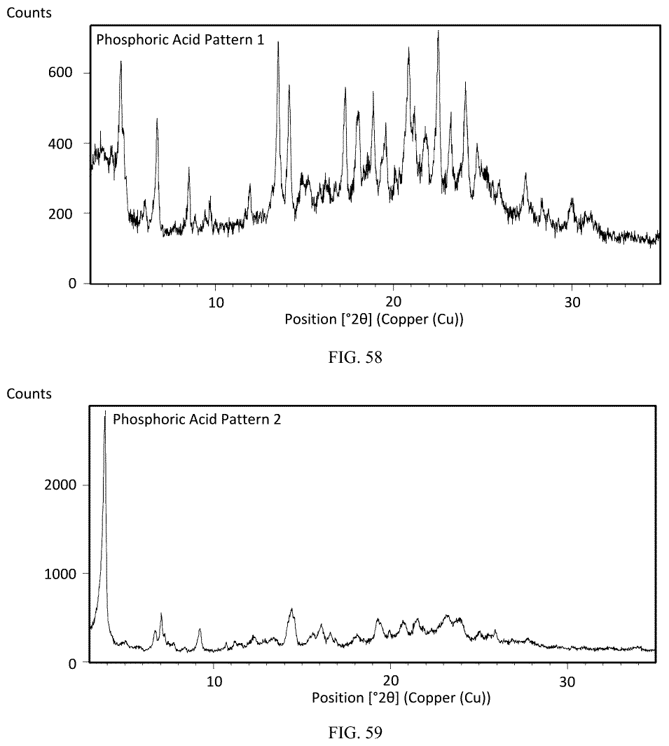

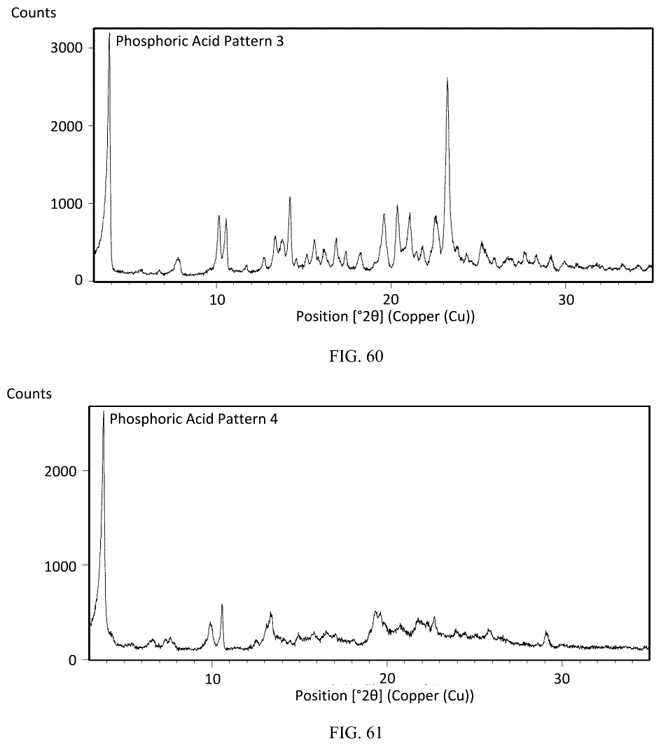

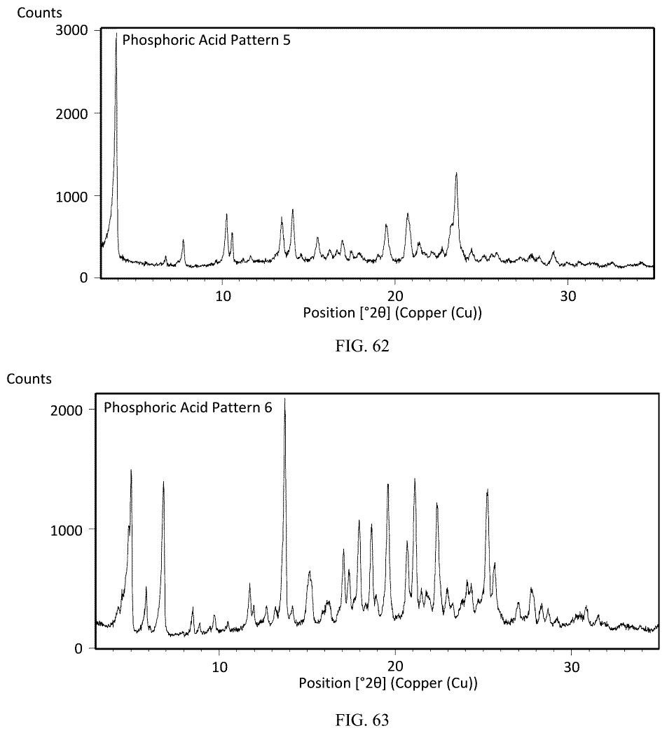

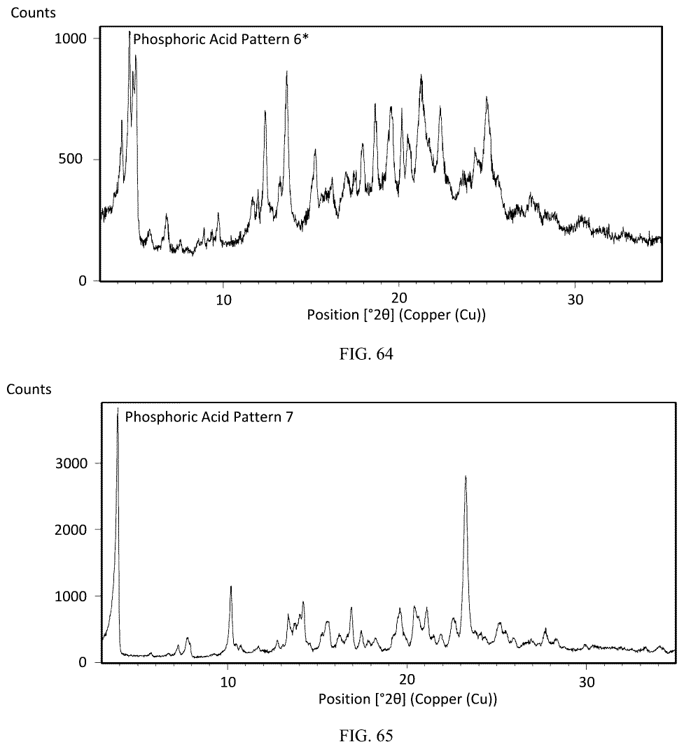

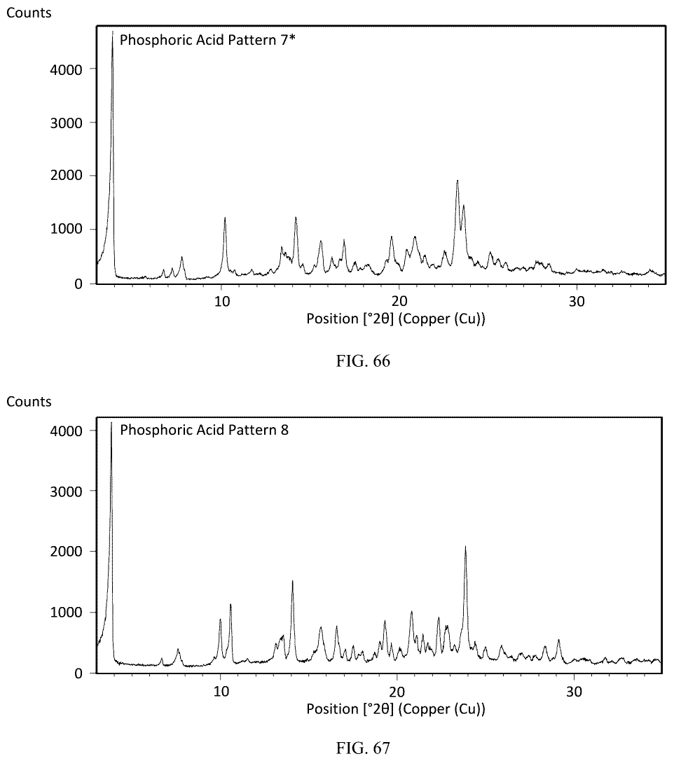

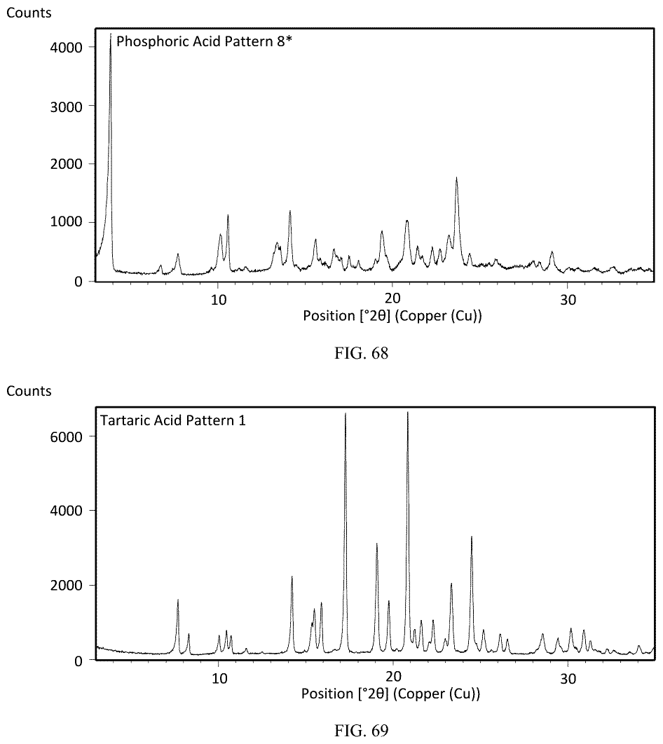

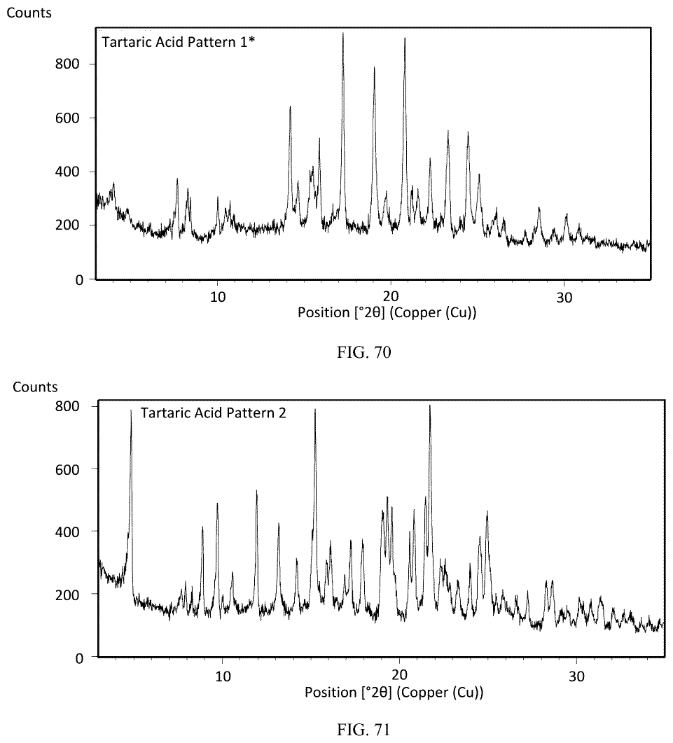

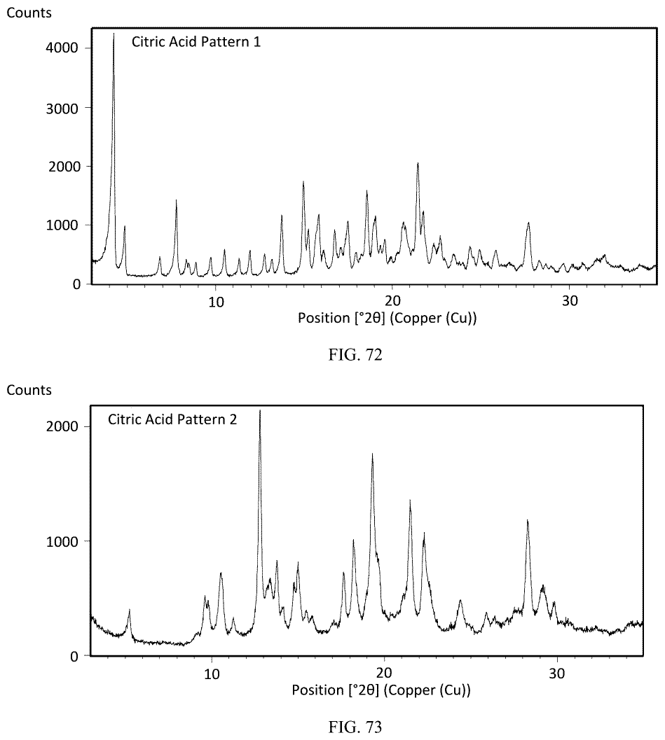

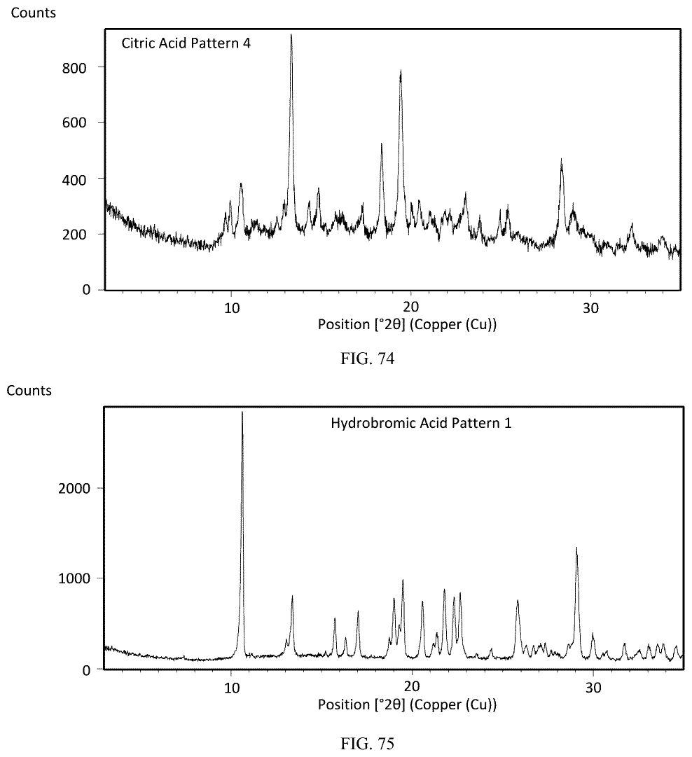

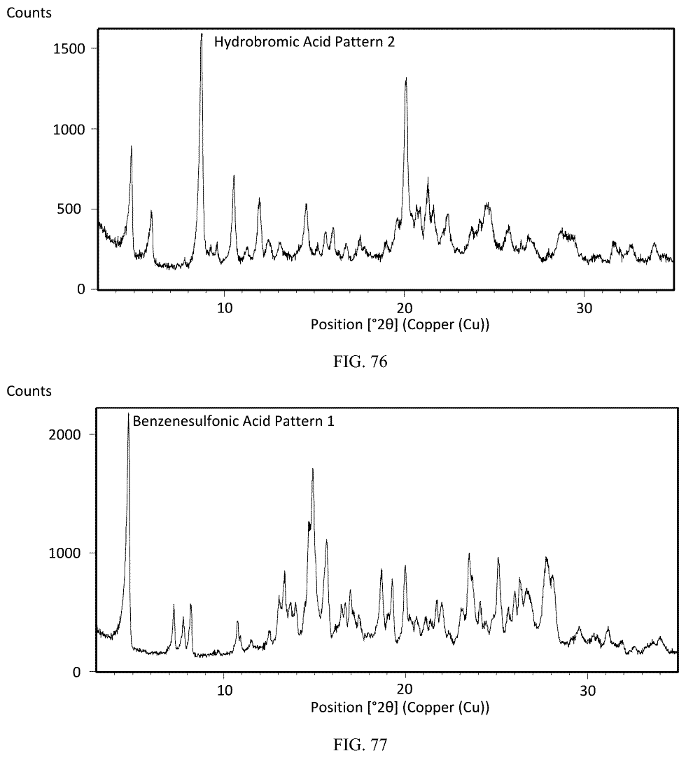

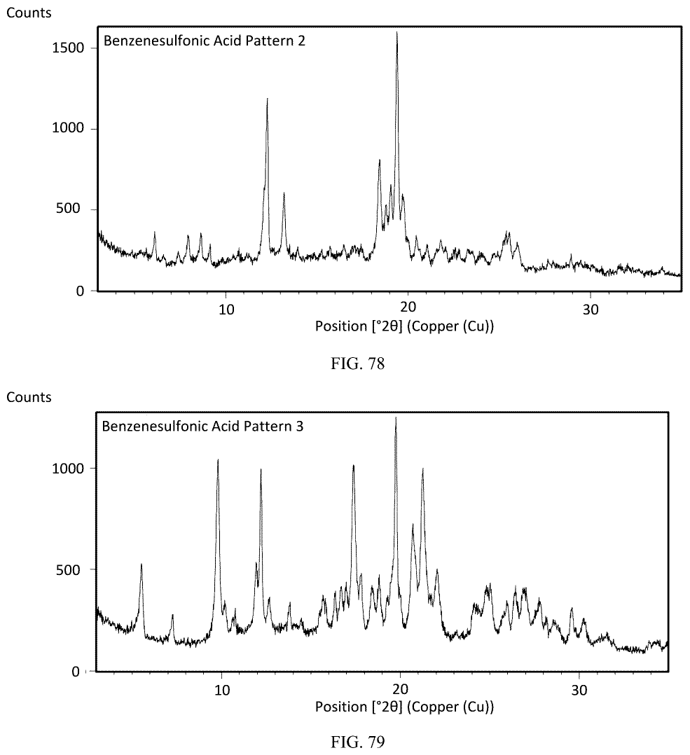

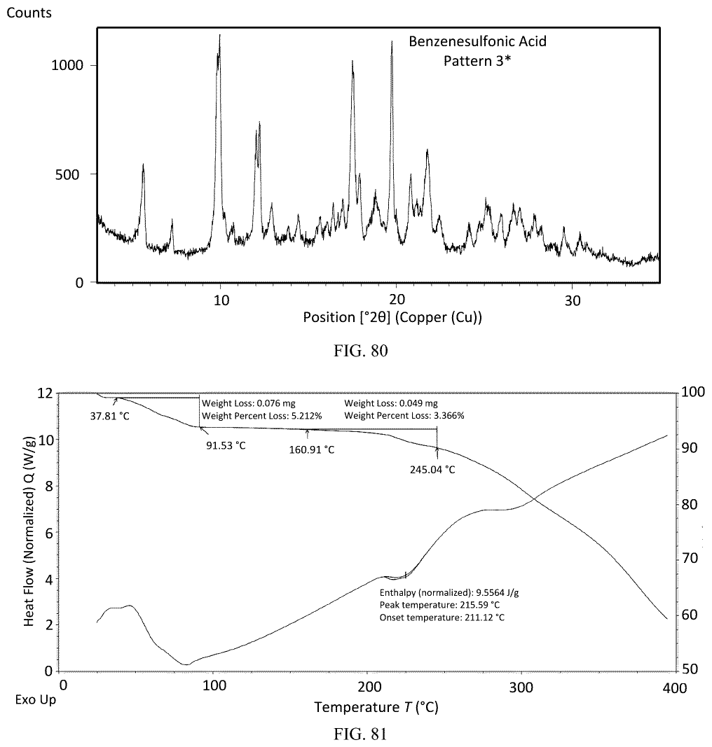

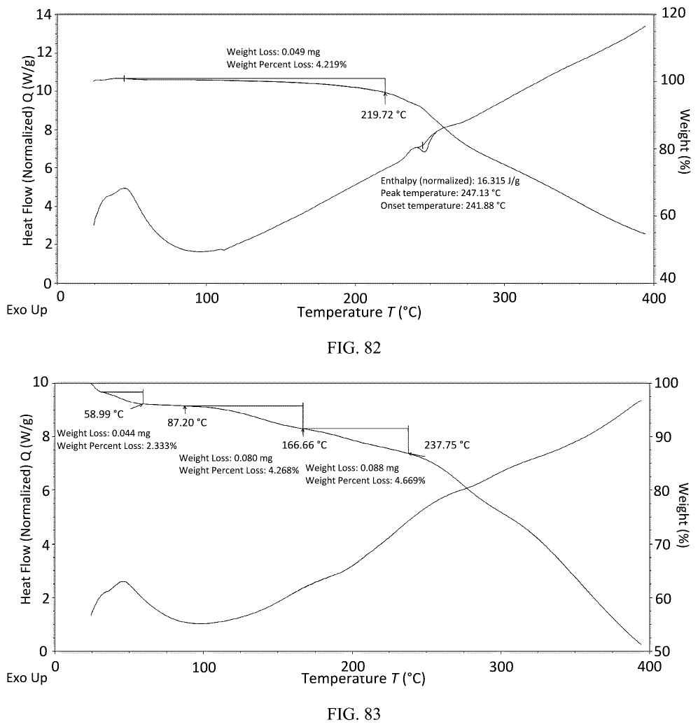

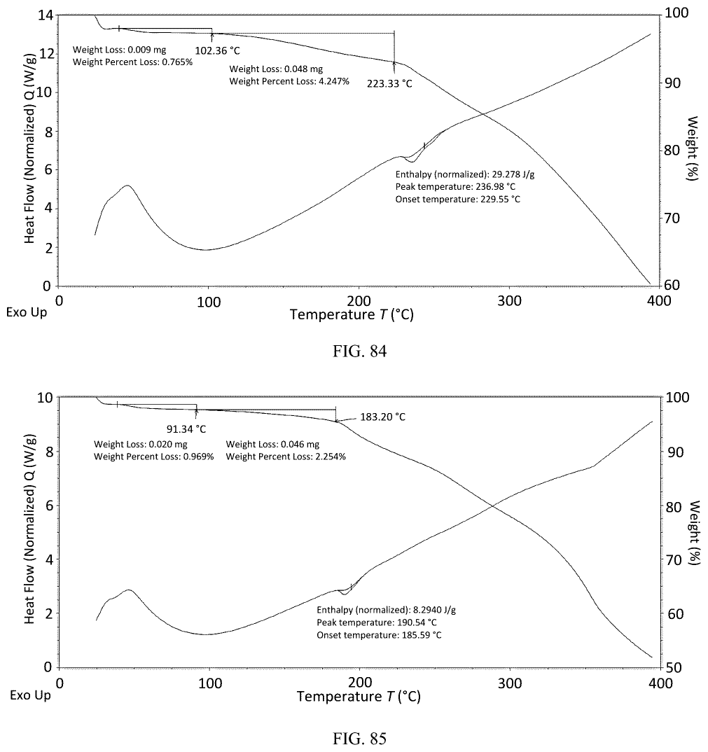

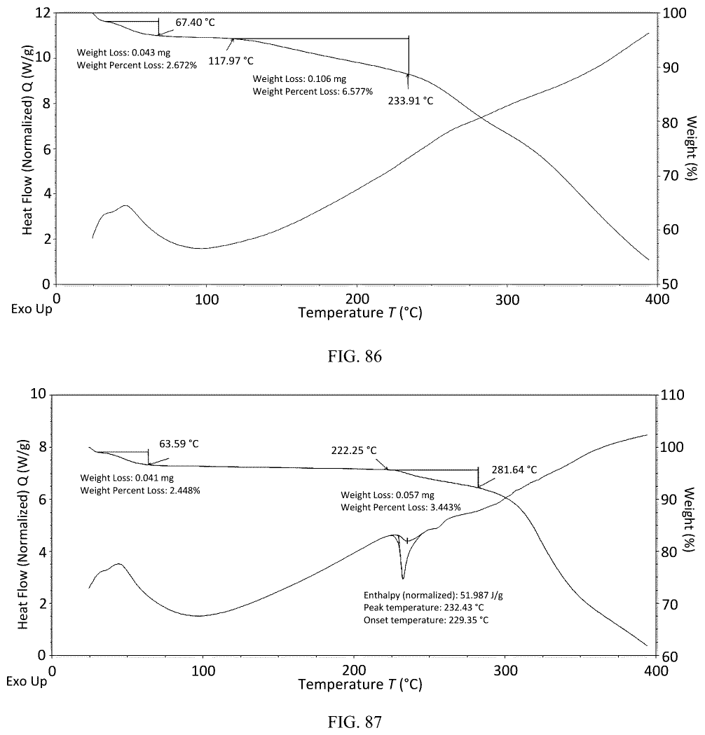

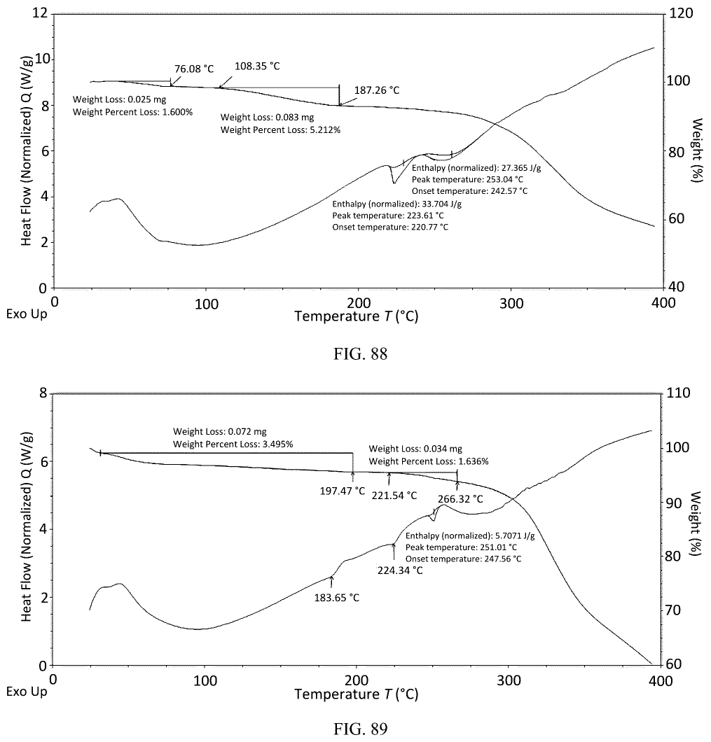

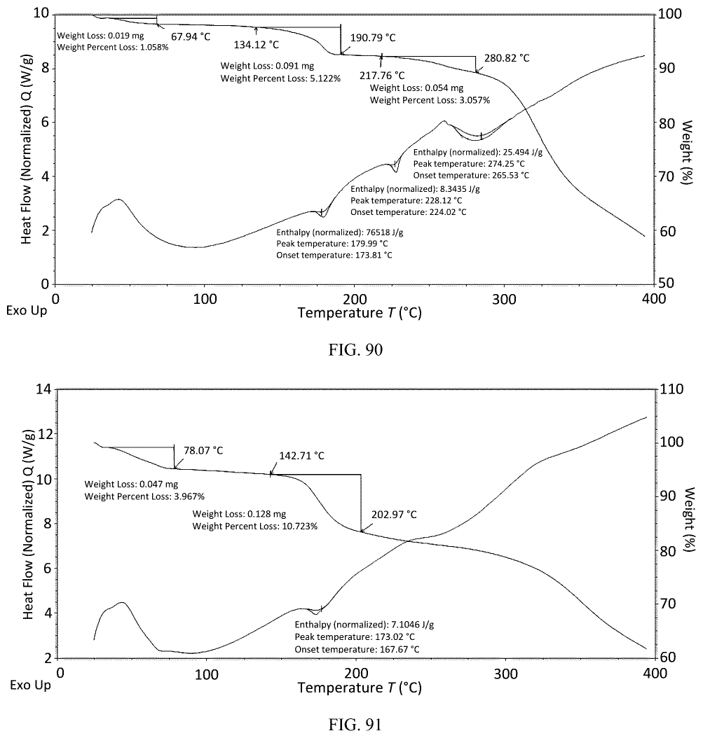

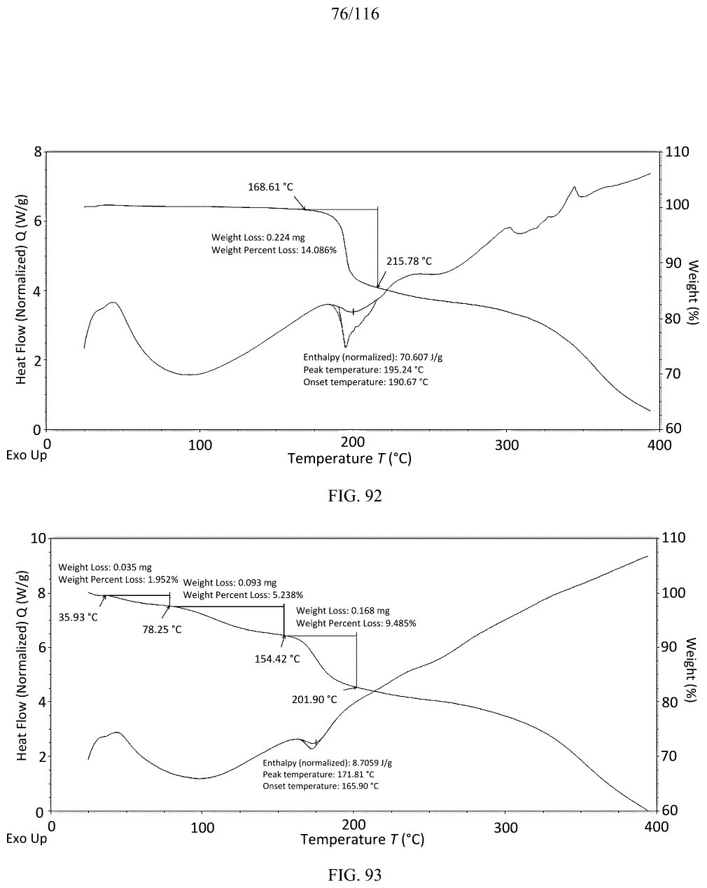

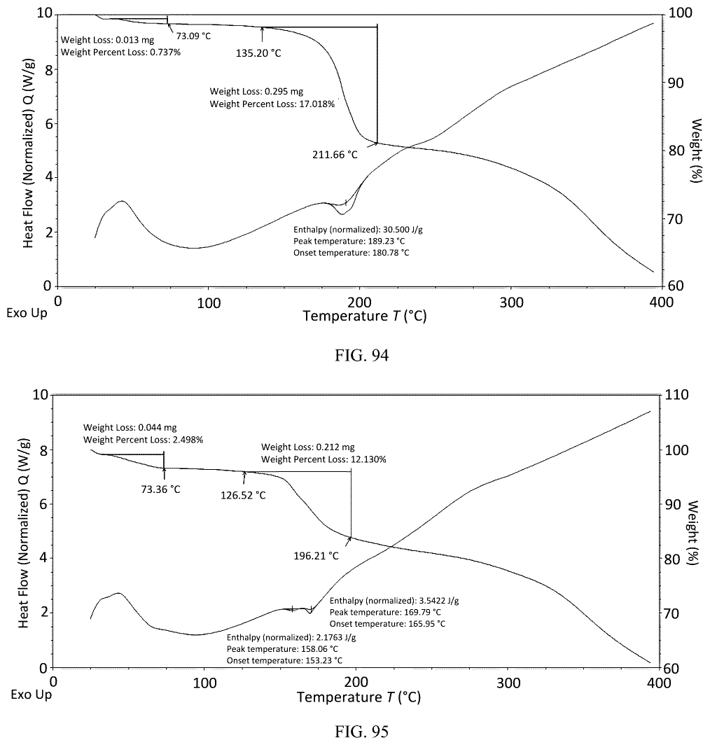

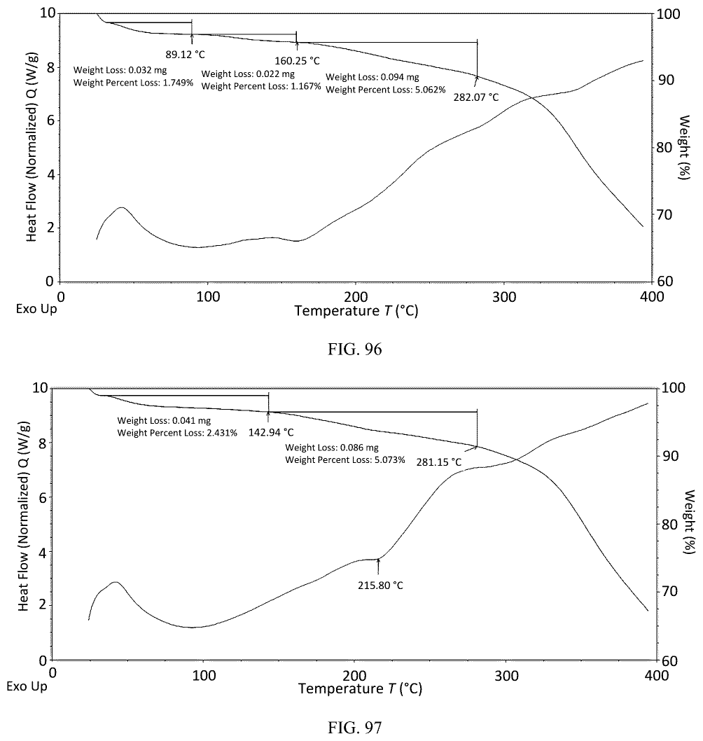

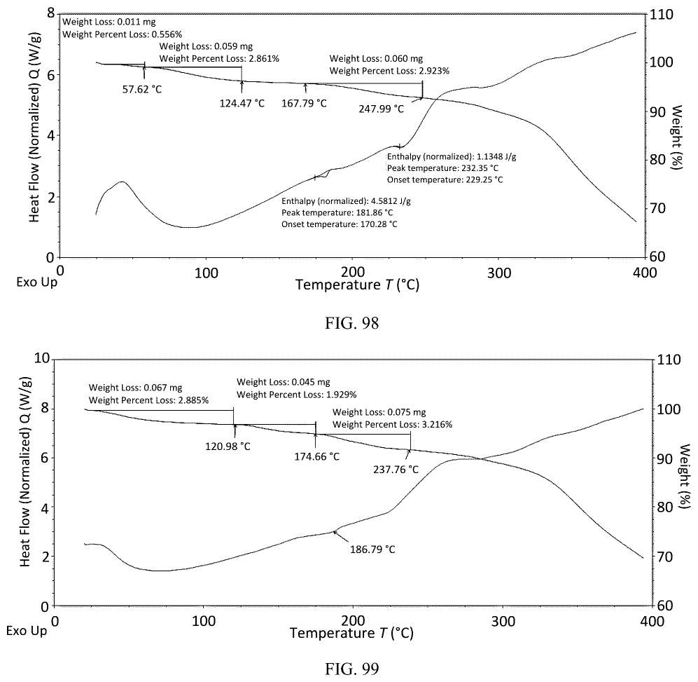

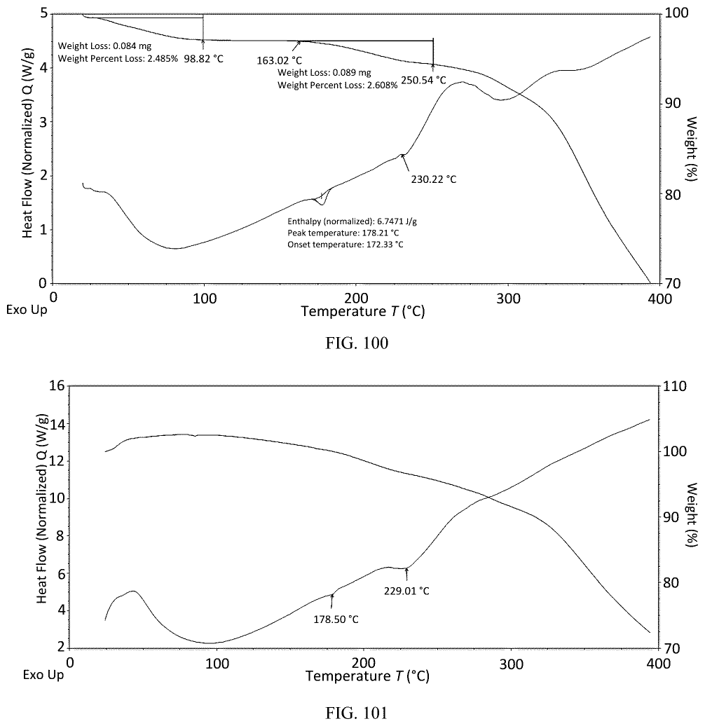

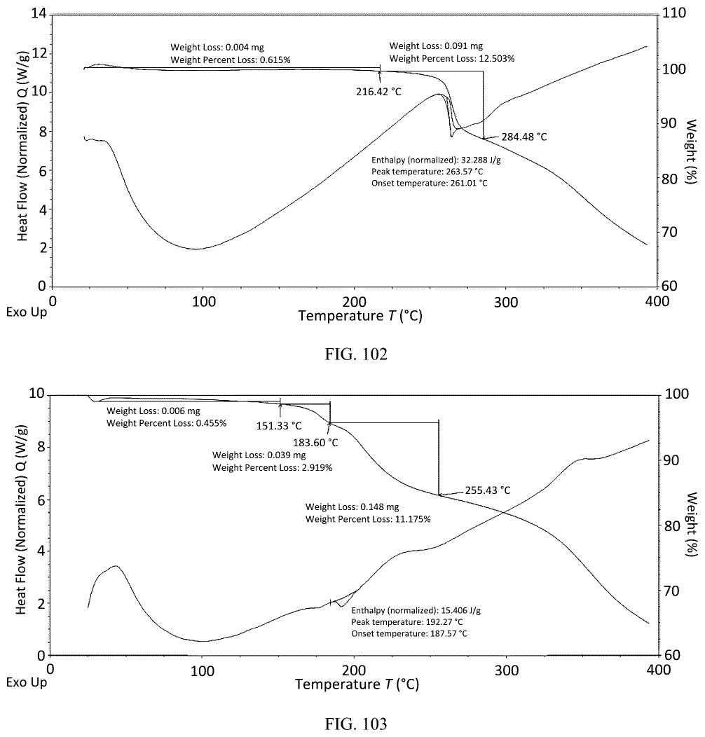

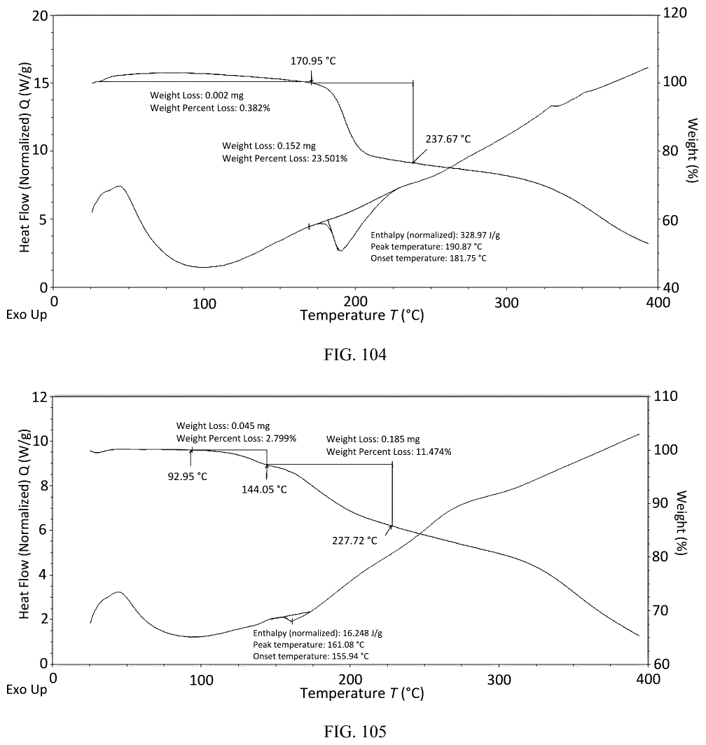

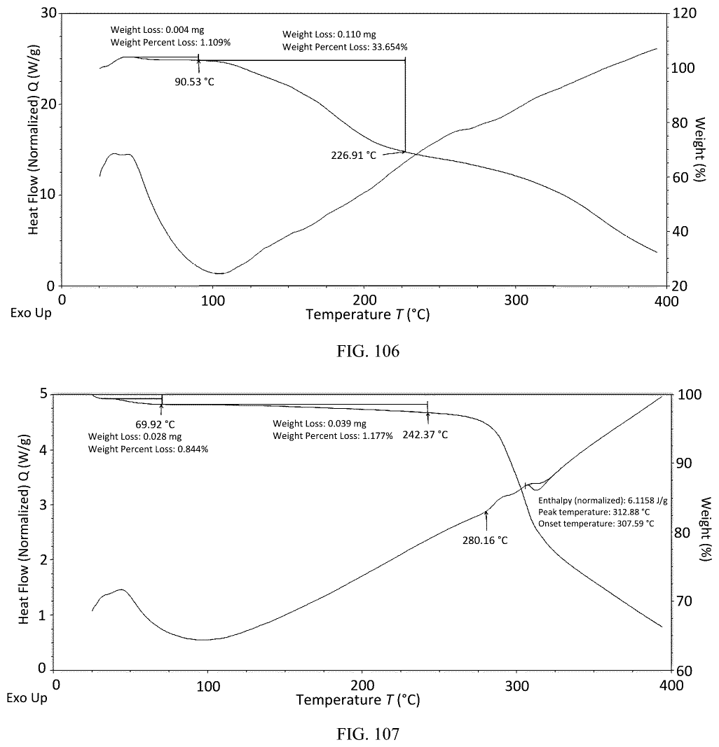

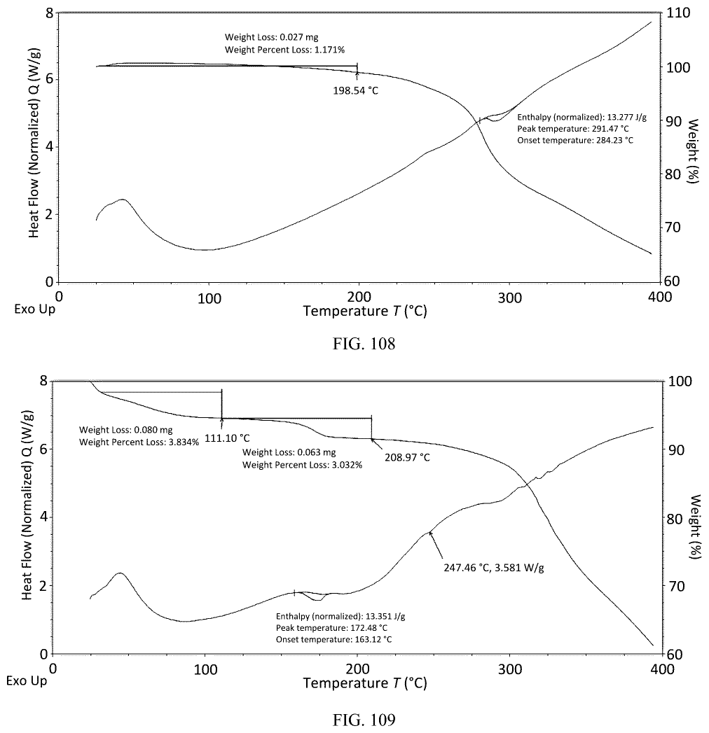

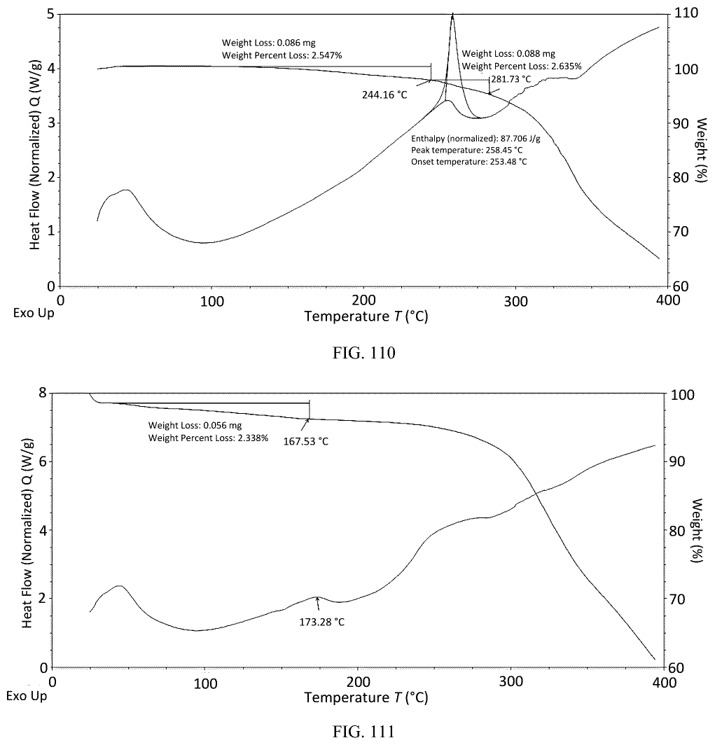

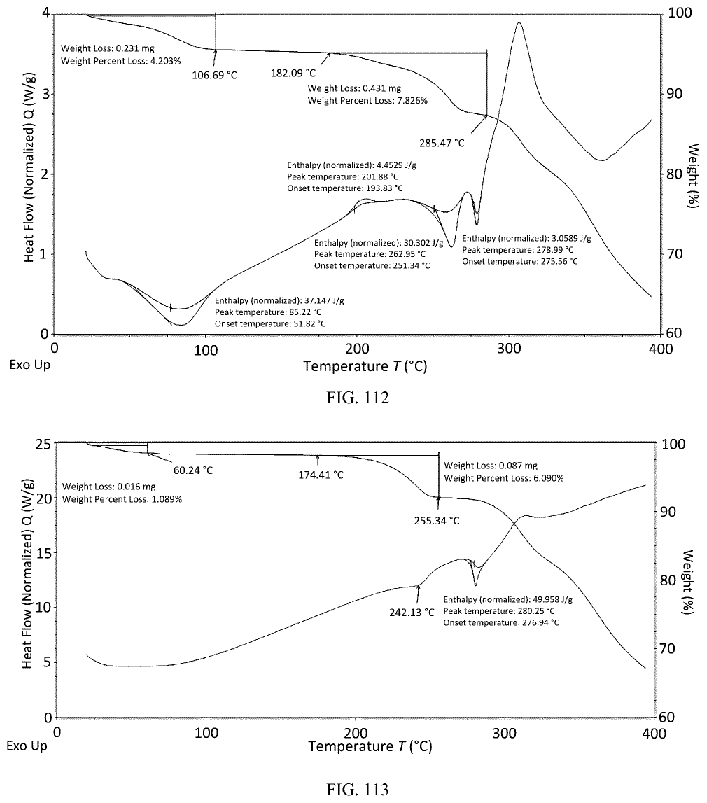

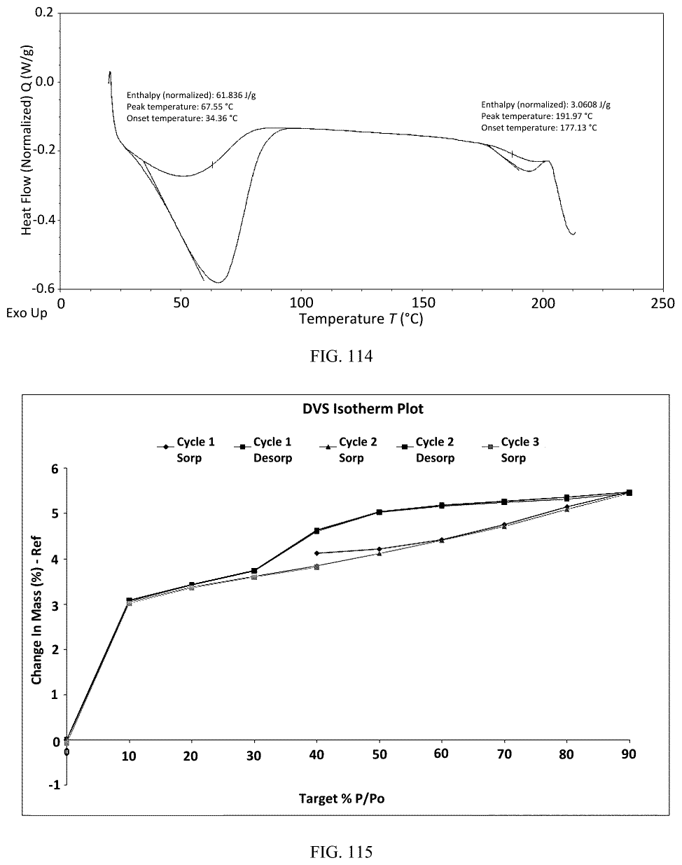

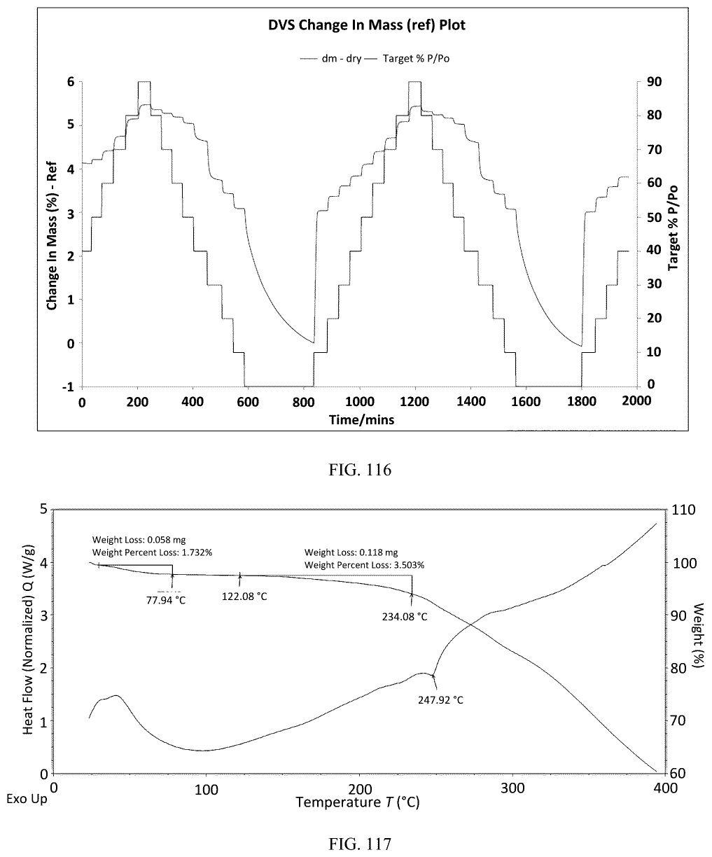

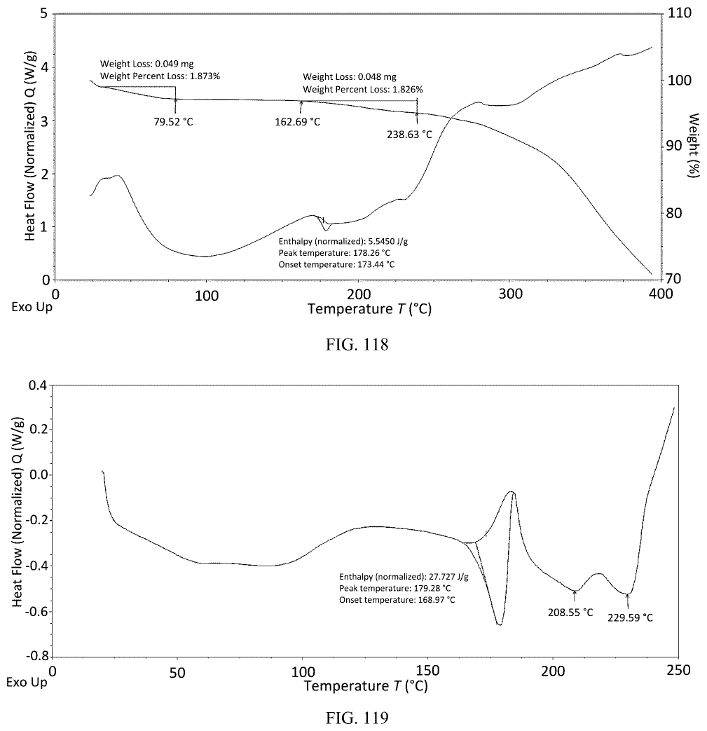

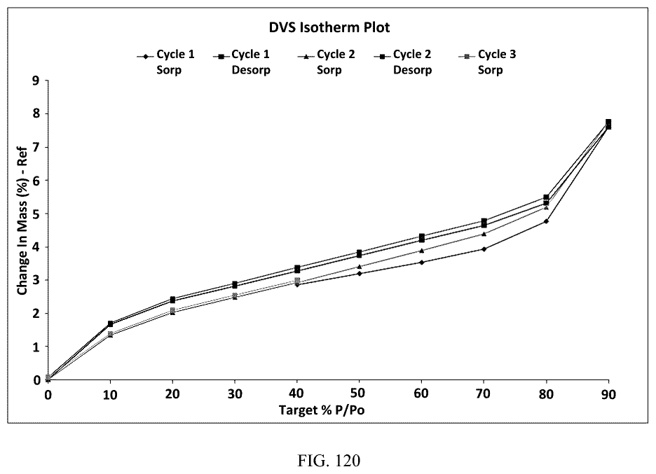

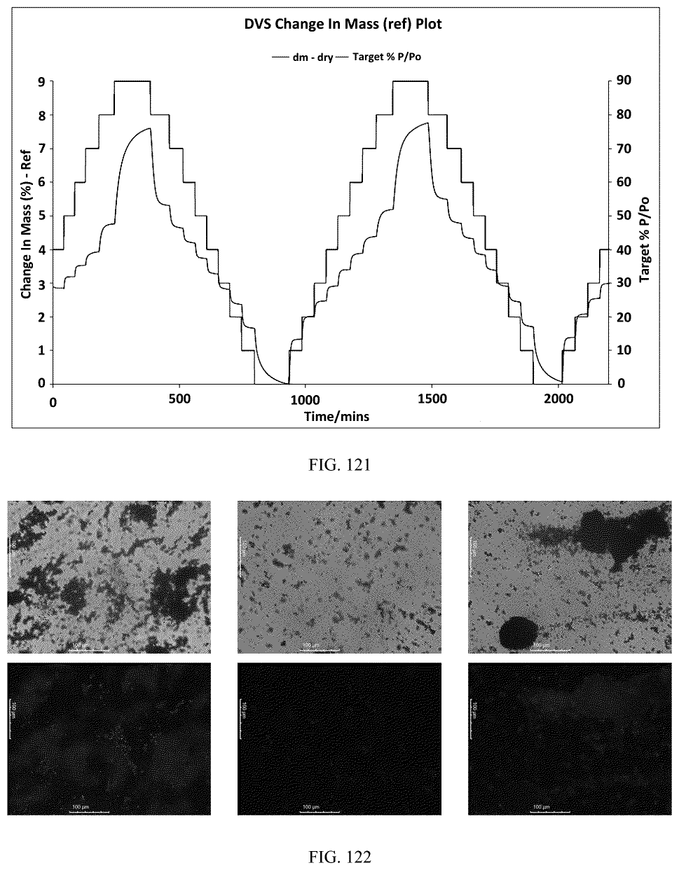

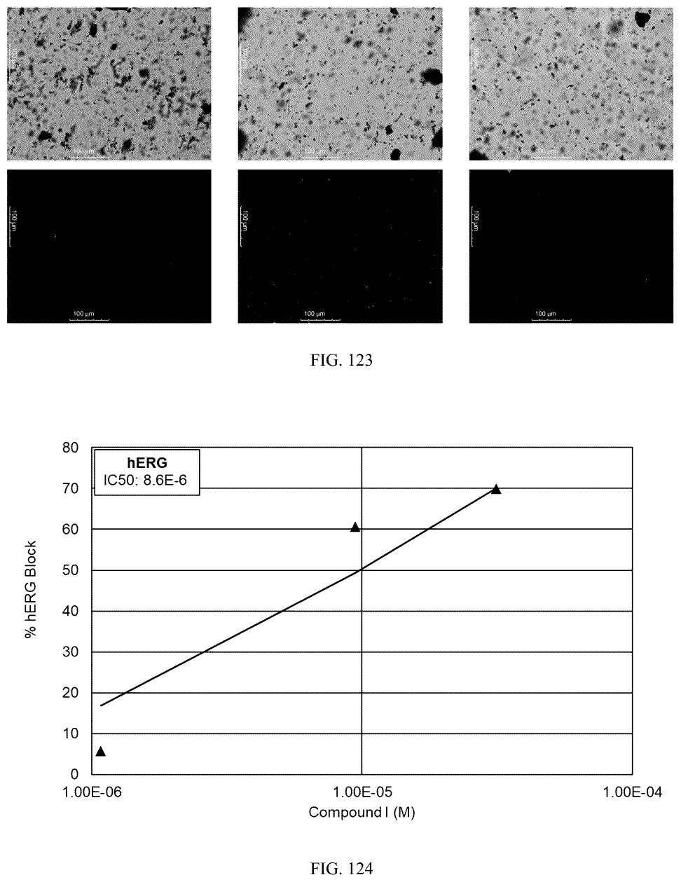

The above technique was used to generate images in FIGS. 32-80.

Table 14 below provides the results of the XRPD performed on free base Pattern 1. The XRPD exhibited sharp peaks, indicating the sample was composed of crystalline material. Significant peaks were observed in the XRPD on free base Pattern 1 at about 10.3±0.2°, about 11.9±0.2°, about 16.3±0.2°, about 17.8±0.2°, about 19.3±0.2°, about 22.4±0.2°, about 23.0±0.2°, about 24.1±0.2°, about 24.7±0.2°, and about 30.0±0.2°.

TABLE 14
XRPD Peak List for Free Base Pattern 1

| Pos. [°2θ] | d-spacing [Å] | Rel. Int. [%] |
|---|---|---|
| 5.9712 | 14.80155 | 0.83 |
| 7.5827 | 11.65904 | 3.56 |
| 7.9469 | 11.12554 | 4.92 |
| 9.2323 | 9.57920 | 21.90 |
| 10.2795 | 8.60566 | 29.57 |
| 11.6936 | 7.56793 | 21.12 |
| 11.9298 | 7.41861 | 23.42 |
| 12.1024 | 7.31322 | 21.21 |
| 13.5298 | 6.54469 | 2.18 |
| 14.3305 | 6.18077 | 17.66 |
| 15.3743 | 5.76342 | 22.45 |
| 15.9459 | 5.55810 | 7.15 |
| 16.3483 | 5.42216 | 29.50 |
| 16.6460 | 5.32587 | 7.96 |
| 17.5142 | 5.06376 | 18.17 |
| 17.7904 | 4.98577 | 78.23 |
| 18.5064 | 4.79444 | 4.13 |
| 19.2658 | 4.60715 | 38.92 |
| 19.6920 | 4.50838 | 11.46 |
| 20.4997 | 4.33255 | 12.54 |
| 20.9684 | 4.23675 | 9.09 |
| 21.4398 | 4.14464 | 5.51 |
| 21.9887 | 4.04241 | 21.80 |
| 22.3775 | 3.97305 | 100.00 |
| 22.9949 | 3.86775 | 96.41 |
| 24.0653 | 3.69809 | 25.79 |
| 24.6907 | 3.60582 | 39.40 |
| 26.5275 | 3.36017 | 12.17 |
| 28.765 | 3.10363 | 14.83 |
| 29.9943 | 2.97923 | 26.53 |
| 31.1455 | 2.87169 | 8.90 |
| 31.9985 | 2.79705 | 9.80 |
| 33.8275 | 2.64989 | 10.43 |

Table 15 below provides the results of the XRPD performed on free base Pattern 2. The XRPD exhibited sharp peaks, indicating the sample was composed of crystalline material. Significant peaks were observed in the XRPD on free base Pattern 2 at about 7.8±0.2°, about 13.6±0.2°, about 15.0±0.2°, about 15.7±0.2°, about 16.4±0.2°, about 18.6±0.2°, about 21.3±0.2°, about 21.7±0.2°, about 22.0±0.2°, about 23.4±0.2°, and about 31.1±0.2°.

TABLE 15
XRPD Peak List for Free Base Pattern 2

| Pos. [°2θ] | d-spacing [Å] | Rel. Int. [%] |
|---|---|---|
| 7.7568 | 11.39772 | 38.05 |
| 13.5745 | 6.52324 | 30.78 |
| 13.9694 | 6.33970 | 29.33 |
| 14.5670 | 6.08097 | 9.78 |
| 15.0256 | 5.89639 | 89.77 |
| 15.5496 | 5.69882 | 26.41 |
| 15.7535 | 5.62552 | 43.24 |
| 16.4438 | 5.39089 | 50.32 |
| 17.1552 | 5.16892 | 15.75 |
| 17.7188 | 5.00576 | 7.40 |
| 18.6483 | 4.75828 | 100.00 |
| 18.9379 | 4.68616 | 15.07 |
| 20.1626 | 4.40422 | 6.87 |
| 20.5725 | 4.31738 | 29.57 |
| 21.3372 | 4.16434 | 45.64 |
| 21.7162 | 4.09251 | 59.85 |
| 21.9741 | 4.04171 | 41.94 |
| 22.0399 | 4.03982 | 34.31 |
| 23.4294 | 3.79385 | 86.83 |
| 24.4098 | 3.64365 | 24.79 |
| 24.6782 | 3.60463 | 27.21 |
| 27.0893 | 3.28903 | 25.22 |
| 27.3732 | 3.25555 | 20.56 |
| 27.7540 | 3.21174 | 18.43 |
| 29.0189 | 3.07456 | 10.71 |
| 29.6822 | 3.00735 | 10.63 |
| 30.5336 | 2.92540 | 29.93 |
| 31.0805 | 2.87516 | 32.47 |
| 31.4921 | 2.83851 | 11.14 |
| 32.5073 | 2.75215 | 6.23 |
| 33.4417 | 2.67736 | 9.41 |
| 33.8805 | 2.64368 | 7.45 |

Table 16 below provides the results of the XRPD performed on free base Pattern 3. The XRPD exhibited sharp peaks, indicating the sample was composed of crystalline material. Significant peaks were observed in the XRPD on free base Pattern 3 at about 9.1±0.2°, about 9.2±0.2°, about 17.1±0.2°, about 18.2±0.2°, about 19.5±0.2°, about 20.2±0.2°, about 24.7±0.2°, about 25.3±0.2°, about 28.0±0.2°, about 28.2±0.2°, about 28.6±0.2°, and about 32.0±0.2°.

TABLE 16
XRPD Peak List for Free Base Pattern 3

| Pos. | d-spacing | Rel. Int. | Pos. | d-spacing | Rel. Int. |
|---|---|---|---|---|---|
| [°2θ] | [Å] | [%] | [°2θ] | [Å] | [%] |
| 4.3891 | 20.13251 | 3.53 | 18.2386 | 4.86423 | 50.17 |
| 4.8356 | 18.27461 | 4.32 | 18.6956 | 4.74635 | 22.46 |
| 6.3970 | 13.81721 | 4.47 | 19.5121 | 4.54954 | 31.45 |
| 6.7088 | 13.17580 | 6.01 | 20.1836 | 4.39967 | 48.43 |
| 7.7896 | 11.34984 | 3.79 | 20.5038 | 4.33168 | 14.57 |
| 9.1051 | 9.71276 | 50.79 | 21.0094 | 4.22858 | 26.99 |
| 9.2342 | 9.57731 | 45.36 | 21.6313 | 4.10839 | 9.32 |
| 9.3191 | 9.49023 | 29.58 | 22.4790 | 3.95534 | 4.44 |
| 10.8993 | 8.11761 | 7.93 | 23.3313 | 3.81275 | 2.96 |
| 11.1615 | 7.92750 | 15.91 | 24.7234 | 3.60112 | 100.00 |
| 11.3440 | 7.80037 | 25.56 | 25.2986 | 3.52054 | 69.98 |
| 11.6043 | 7.62599 | 18.74 | 25.9622 | 3.43204 | 6.23 |
| 12.4824 | 7.09142 | 6.61 | 27.0807 | 3.29278 | 4.73 |
| 13.1462 | 6.73480 | 6.30 | 27.7779 | 3.21169 | 11.70 |
| 13.4866 | 6.56554 | 6.53 | 28.0007 | 3.18664 | 68.41 |
| 14.2810 | 6.20210 | 8.44 | 28.2546 | 3.15858 | 69.14 |
| 14.9580 | 5.92287 | 5.40 | 28.6517 | 3.11571 | 68.08 |
| 16.0684 | 5.51598 | 8.38 | 29.2988 | 3.04835 | 5.61 |
| 16.2890 | 5.44178 | 14.20 | 31.9901 | 2.79545 | 39.99 |
| 16.6675 | 5.31903 | 20.39 | 32.0791 | 2.79482 | 17.67 |
| 17.1196 | 5.17957 | 67.55 | 34.2152 | 2.61857 | 2.46 |
| 17.7515 | 4.99662 | 8.59 | 23.3313 | 3.81275 | 2.96 |

Table 17 below provides the results of the XRPD performed on hydrochloric acid salt Pattern 1. The XRPD exhibited sharp peaks, indicating the sample was composed of crystalline material. Significant peaks were observed in the XRPD on hydrochloric acid salt Pattern 1 at about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

TABLE 17
XRPD Diffractogram of Hydrochloric Acid Salt Pattern 1

| Pos. [°2θ] | d-spacing [Å] | Rel. Int. [%] |
|---|---|---|
| 7.2615 | 12.17401 | 100.00 |
| 9.6841 | 9.13334 | 7.93 |
| 10.7529 | 8.22781 | 69.43 |
| 11.5051 | 7.69149 | 17.38 |
| 13.8015 | 6.41647 | 8.02 |
| 14.4381 | 6.13494 | 39.44 |
| 15.4725 | 5.72706 | 73.66 |
| 15.6527 | 5.66152 | 95.25 |
| 16.65 | 5.3246 | 84.08 |
| 16.9334 | 5.23611 | 76.07 |
| 17.5503 | 5.05343 | 15.7 |
| 18.3183 | 4.84325 | 24.15 |
| 18.4902 | 4.79863 | 29.43 |
| 19.2563 | 4.60941 | 98.56 |
| 19.9766 | 4.4448 | 93.07 |
| 20.7322 | 4.28449 | 24.1 |
| 21.5114 | 4.13101 | 18.75 |
| 21.9072 | 4.05727 | 37.26 |
| 22.4238 | 3.96495 | 12.18 |
| 22.7874 | 3.90251 | 11.47 |
| 23.1269 | 3.84598 | 13.54 |
| 23.482 | 3.78862 | 13.28 |
| 24.0529 | 3.69996 | 77.17 |
| 25.1156 | 3.54578 | 99.51 |
| 25.6143 | 3.47786 | 30.94 |
| 25.9482 | 3.43386 | 86.83 |
| 26.8976 | 3.31477 | 9.52 |
| 27.855 | 3.20297 | 68.91 |
| 28.6379 | 3.11717 | 8.79 |
| 29.1656 | 3.06196 | 12.79 |
| 29.4753 | 3.0305 | 12.38 |
| 30.3589 | 2.94427 | 21.21 |
| 31.2234 | 2.86469 | 12.35 |
| 31.9017 | 2.80532 | 18.05 |
| 32.5486 | 2.75103 | 7.01 |
| 33.0901 | 2.70723 | 5.7 |
| 33.9409 | 2.6413 | 19.79 |

In certain embodiments, the morphic form is Hydrochloric Acid Salt Pattern 1 and is characterized by an XRPD pattern comprising at least 2 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

In certain embodiments, the morphic form is Hydrochloric Acid Salt Pattern 1 and is characterized by an XRPD pattern comprising at least 3 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

In certain embodiments, the morphic form is Hydrochloric Acid Salt Pattern 1 and is characterized by an XRPD pattern comprising at least 4 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

In certain embodiments, the morphic form is Hydrochloric Acid Salt Pattern 1 and is characterized by an XRPD pattern comprising at least 5 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

In certain embodiments, the morphic form is Hydrochloric Acid Salt Pattern 1 and is characterized by an XRPD pattern comprising at least 6 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

In certain embodiments, the morphic form is Hydrochloric Acid Salt Pattern 1 and is characterized by an XRPD pattern comprising at least 7 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

In certain embodiments, the morphic form is Hydrochloric Acid Salt Pattern 1 and is characterized by an XRPD pattern comprising at least 8 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°. In certain embodiments, the morphic form is Hydrochloric Acid Salt Pattern 1 and is characterized by an XRPD pattern comprising at least 9 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°. In certain embodiments, the morphic form is Hydrochloric Acid Salt Pattern 1 and is characterized by an XRPD pattern comprising at least 10 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

In certain embodiments, the morphic form is Hydrochloric Acid Salt Pattern 1 and is characterized by an XRPD pattern comprising at least 11 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

In certain embodiments, the morphic form is Hydrochloric Acid Salt Pattern 1 and is characterized by an XRPD pattern comprising at least 12 peaks selected from about 7.3±0.2°, about 10.7±0.2°, about 15.5±0.2°, about 15.6±0.2°, about 16.6±0.2°, about 16.9±0.2°, about 19.3±0.2°, about 20.0±0.2°, about 24.0±0.2°, about 25.1±0.2°, about 25.9±0.2°, and about 27.9±0.2°.

Table 18 below provides the results of the XRPD performed on hydrochloric acid salt Pattern 2. The XRPD exhibited sharp peaks, indicating the sample was composed of crystalline material. Significant peaks were observed in the XRPD on hydrochloric acid salt Pattern 2 at about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

TABLE 18
XRPD Peak List for Hydrochloric Acid Salt Pattern 2

| Pos. [°2θ] | d-spacing [Å] | Rel. Int. [%] |
|---|---|---|
| 7.4585 | 11.85289 | 5.84 |
| 9.6068 | 9.20668 | 6.98 |
| 10.6068 | 8.34083 | 100.00 |
| 11.2412 | 7.87148 | 6.05 |
| 13.4321 | 6.59208 | 22.37 |
| 14.9488 | 5.92648 | 6.90 |
| 15.8565 | 5.58923 | 20.08 |
| 16.5185 | 5.36668 | 2.57 |
| 17.0772 | 5.19234 | 15.23 |
| 19.0183 | 4.66654 | 14.55 |
| 19.3547 | 4.58618 | 22.26 |
| 19.6631 | 4.51496 | 19.18 |
| 20.7812 | 4.27449 | 24.71 |
| 21.4593 | 4.14093 | 7.36 |
| 21.7384 | 4.08838 | 13.23 |
| 22.3108 | 3.98478 | 17.98 |
| 22.718 | 3.91426 | 27.93 |
| 24.4411 | 3.64208 | 5.90 |
| 25.9552 | 3.43295 | 18.52 |
| 26.3952 | 3.37671 | 2.72 |
| 26.8947 | 3.31513 | 3.52 |
| 27.4219 | 3.25257 | 4.48 |
| 27.8299 | 3.20581 | 3.55 |
| 28.5881 | 3.12249 | 3.21 |
| 29.1485 | 3.06372 | 31.92 |
| 30.0354 | 2.97524 | 4.75 |
| 30.6666 | 2.91543 | 1.84 |
| 31.7873 | 2.81515 | 5.56 |
| 32.6008 | 2.74674 | 3.48 |
| 33.3633 | 2.68569 | 3.30 |
| 34.2028 | 2.62167 | 7.09 |
| 34.7451 | 2.58198 | 5.98 |

In certain embodiments, the morphic form is Hydrochloric Acid Salt Pattern 2 and is characterized by an XRPD pattern comprising at least 2 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

In certain embodiments, the morphic form is Hydrochloric Acid Salt Pattern 2 and is characterized by an XRPD pattern comprising at least 3 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

In certain embodiments, the morphic form is Hydrochloric Acid Salt Pattern 2 and is characterized by an XRPD pattern comprising at least 4 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

In certain embodiments, the morphic form is Hydrochloric Acid Salt Pattern 2 and is characterized by an XRPD pattern comprising at least 5 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

In certain embodiments, the morphic form is Hydrochloric Acid Salt Pattern 2 and is characterized by an XRPD pattern comprising at least 6 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

In certain embodiments, the morphic form is Hydrochloric Acid Salt Pattern 2 and is characterized by an XRPD pattern comprising at least 7 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

In certain embodiments, the morphic form is Hydrochloric Acid Salt Pattern 2 and is characterized by an XRPD pattern comprising at least 8 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

In certain embodiments, the morphic form is Hydrochloric Acid Salt Pattern 2 and is characterized by an XRPD pattern comprising at least 9 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

In certain embodiments, the morphic form is Hydrochloric Acid Salt Pattern 2 and is characterized by an XRPD pattern comprising at least 10 peaks selected from about 10.6±0.2°, about 13.4±0.2°, about 15.9±0.2°, about 19.3±0.2°, about 19.7±0.2°, about 20.8±0.2°, about 22.3±0.2°, about 22.7±0.2°, about 25.9±0.2°, and about 29.1±0.2°.

Table 19 below provides the results of the XRPD performed on hydrochloric acid salt Pattern 3. The XRPD exhibited sharp peaks, indicating the sample was composed of crystalline material. Significant peaks were observed in the XRPD on hydrochloric acid salt Pattern 3 at about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

TABLE 19
XRPD Peak List for Hydrochloric Acid Salt Pattern 3

| Pos. [°2θ] | d-spacing [Å] | Rel. Int. [%] |
|---|---|---|
| 4.8797 | 18.10975 | 58.04 |
| 7.9193 | 11.16425 | 13.01 |
| 8.9067 | 9.92864 | 41.83 |
| 9.7378 | 9.08307 | 45.06 |
| 11.9745 | 7.39102 | 48.12 |
| 13.182 | 6.71656 | 38.54 |
| 15.2293 | 5.81796 | 94.77 |
| 16.0743 | 5.51398 | 37.44 |
| 16.8998 | 5.24646 | 19.29 |
| 17.9177 | 4.95063 | 41.21 |
| 18.9489 | 4.68349 | 20.89 |
| 19.2719 | 4.60571 | 50.69 |
| 19.6161 | 4.52565 | 34.61 |
| 20.6194 | 4.30766 | 33.91 |
| 21.4685 | 4.13918 | 60.60 |
| 21.6953 | 4.09642 | 100.00 |
| 22.3548 | 3.97703 | 22.59 |
| 22.5729 | 3.9391 | 27.82 |
| 23.3266 | 3.81035 | 5.65 |
| 23.976 | 3.71166 | 24.56 |
| 24.58 | 3.62181 | 26.24 |
| 24.879 | 3.57896 | 42.96 |
| 25.4475 | 3.49737 | 10.12 |
| 25.8127 | 3.45157 | 9.52 |
| 26.2585 | 3.39117 | 4.57 |
| 26.6056 | 3.35048 | 7.60 |
| 27.1722 | 3.28189 | 15.31 |
| 28.2811 | 3.15568 | 15.54 |
| 28.6256 | 3.11849 | 14.80 |
| 29.5077 | 3.02724 | 7.45 |
| 30.2892 | 2.95089 | 9.02 |
| 30.7328 | 2.9093 | 9.85 |
| 31.2402 | 2.86319 | 10.81 |
| 31.4885 | 2.84118 | 8.79 |
| 32.0726 | 2.79076 | 10.51 |
| 33.0457 | 2.71077 | 7.26 |
| 33.6544 | 2.66312 | 3.10 |
| 34.1438 | 2.62606 | 4.20 |

In certain embodiments, the morphic form is Hydrochloric Acid Salt Pattern 3 and is characterized by an XRPD pattern comprising at least 2 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 3 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 4 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 5 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 6 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 7 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 8 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 9 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 10 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

In certain embodiments, the morphic form is Hydrochloric Acid Salt Pattern 3 and is characterized by an XRPD pattern comprising at least 11 peaks selected from about 4.9±0.2°, about 8.9±0.2°, about 9.7±0.2°, about 12.0±0.2°, about 13.2±0.2°, about 15.2±0.2°, about 17.9±0.2°, about 19.3±0.2°, about 21.5±0.2°, about 21.7±0.2°, and about 24.9±0.2°.

Table 20 below provides the results of the XRPD performed on sulfuric acid salt Pattern 1. The XRPD exhibited sharp peaks, indicating the sample was composed of crystalline material. Significant peaks were observed in the XRPD on sulfuric acid salt Pattern 1 at about 7.1±0.2°, about 7.3±0.2°, about 14.4±0.2°, about 14.8±0.2°, about 15.1±0.2°, about 17.2±0.2°, about 18.9±0.2°, about 20.5±0.2°, about 23.7±0.2°, about 24.4±0.2°, and about 24.7±0.2°.

TABLE 20
XRPD Peak List for Sulfuric Acid Salt Pattern 1

| Pos. [°2θ] | d-spacing [Å] | Rel. Int. [%] |
|---|---|---|
| 7.0872 | 12.47310 | 58.96 |
| 7.2684 | 12.16258 | 63.60 |
| 8.3044 | 10.64734 | 13.40 |
| 8.9306 | 9.90213 | 18.62 |
| 9.4217 | 9.38713 | 6.59 |
| 9.9599 | 8.88099 | 18.65 |
| 10.5526 | 8.38348 | 5.27 |
| 12.1973 | 7.25654 | 15.93 |
| 12.5500 | 7.05337 | 13.96 |
| 13.1182 | 6.74909 | 13.81 |
| 14.4501 | 6.12987 | 37.10 |
| 14.7807 | 5.99351 | 65.01 |
| 15.0915 | 5.87078 | 36.28 |
| 15.7167 | 5.63862 | 6.48 |
| 16.2303 | 5.46133 | 9.41 |
| 16.7609 | 5.28961 | 19.25 |
| 17.2442 | 5.14244 | 100.00 |
| 17.8826 | 4.96027 | 26.54 |
| 18.3424 | 4.83696 | 17.41 |
| 18.8583 | 4.70577 | 49.88 |
| 19.3277 | 4.59255 | 9.72 |
| 19.9506 | 4.45053 | 14.15 |
| 20.5000 | 4.33249 | 30.48 |
| 21.0682 | 4.21691 | 14.77 |
| 21.4816 | 4.13668 | 18.86 |
| 22.0689 | 4.02789 | 25.13 |
| 23.0459 | 3.85612 | 8.91 |
| 23.6679 | 3.75927 | 44.50 |
| 24.3755 | 3.65173 | 99.38 |
| 24.7140 | 3.60247 | 88.85 |
| 25.8971 | 3.44052 | 29.44 |
| 27.1398 | 3.28573 | 17.45 |
| 27.7162 | 3.21870 | 12.75 |
| 28.5339 | 3.12829 | 16.31 |
| 29.0773 | 3.07106 | 12.39 |
| 30.8712 | 2.89657 | 3.57 |
| 31.5930 | 2.83202 | 3.57 |
| 32.8349 | 2.72769 | 6.39 |

Table 21 below provides the results of the XRPD performed on sulfuric acid salt Pattern 1*. (* indicates slight shifts in XRPD peak positions in comparison with Sulfuric Acid Pattern 1.) The XRPD exhibited sharp peaks, indicating the sample was composed of crystalline material. Significant peaks were observed in the XRPD on sulfuric acid salt Pattern 1* at about 7.3±0.2°, about 14.5±0.2°, about 14.8±0.2°, about 15.1±0.2°, about 17.3±0.2°, about 20.5±0.2°, about 22.1±0.2°, about 23.7±0.2°, about 24.3±0.2°, and about 26.0±0.2°.

TABLE 21
XRPD Peak List for Sulfuric Acid Salt Pattern 1*

| Pos. [°2θ] | d-spacing [Å] | Rel. Int. [%] |
|---|---|---|
| 7.2784 | 12.1458 | 59.22 |
| 8.2902 | 10.6656 | 13.59 |
| 8.9558 | 9.8744 | 14.55 |
| 9.4262 | 9.3826 | 6.11 |
| 9.9228 | 8.9142 | 7.86 |
| 10.5123 | 8.4155 | 5.58 |
| 12.1962 | 7.2572 | 13.47 |
| 13.0737 | 6.7720 | 12.96 |
| 14.4577 | 6.1267 | 31.41 |
| 14.7713 | 5.9973 | 26.26 |
| 15.1383 | 5.8527 | 32.90 |
| 15.7114 | 5.6405 | 6.03 |
| 17.2711 | 5.1345 | 93.04 |
| 17.8647 | 4.9652 | 22.58 |
| 18.3347 | 4.8390 | 14.54 |
| 18.8203 | 4.7152 | 20.52 |
| 19.3304 | 4.5919 | 9.07 |
| 19.9896 | 4.4419 | 9.33 |
| 20.5302 | 4.3262 | 28.54 |
| 21.1859 | 4.1937 | 11.62 |
| 21.6111 | 4.1088 | 8.04 |
| 22.0749 | 4.0268 | 24.42 |
| 22.6716 | 3.9189 | 3.99 |
| 23.6751 | 3.7582 | 42.66 |
| 24.3301 | 3.6584 | 100.00 |
| 25.1789 | 3.5370 | 12.18 |
| 25.9818 | 3.4295 | 26.60 |
| 27.0325 | 3.2985 | 7.16 |
| 27.7273 | 3.2174 | 10.81 |
| 28.4907 | 3.1329 | 15.26 |
| 29.1207 | 3.0666 | 10.74 |
| 30.7407 | 2.9086 | 2.42 |
| 31.6261 | 2.8291 | 2.24 |
| 32.5177 | 2.7536 | 2.48 |

Table 22 below provides the results of the XRPD performed on sulfuric acid salt Pattern 2. The XRPD exhibited sharp peaks, indicating the sample was composed of crystalline material. Significant peaks were observed in the XRPD on sulfuric acid salt Pattern 2 at about 12.4±0.2°, about 13.4±0.2°, about 17.7±0.2°, about 18.0±0.2°, about 18.6±0.2°, about 19.1±0.2°, about 20.7±0.2°, about 21.0±0.2°, about 21.8±0.2°, and about 22.5±0.2°.

TABLE 22
XRPD Peak List for Sulfuric Acid Salt Pattern 2

| Pos. [°2θ] | d-spacing [Å] | Rel. Int. [%] |
|---|---|---|
| 6.1743 | 14.31517 | 19.70 |
| 7.4431 | 11.87746 | 22.47 |
| 8.6987 | 10.16565 | 2.04 |
| 9.9771 | 8.85843 | 6.31 |
| 10.3310 | 8.56285 | 18.96 |
| 11.1210 | 7.95624 | 17.83 |
| 11.8173 | 7.48277 | 10.19 |
| 12.3952 | 7.14110 | 96.27 |
| 12.9090 | 6.85233 | 7.04 |
| 13.4282 | 6.59397 | 72.00 |
| 14.0401 | 6.30798 | 18.29 |
| 14.9309 | 5.93355 | 41.08 |
| 15.8081 | 5.60621 | 29.37 |
| 16.6518 | 5.31960 | 11.87 |
| 16.9763 | 5.22299 | 24.72 |
| 17.3380 | 5.11058 | 12.30 |
| 17.6594 | 5.02245 | 70.07 |
| 18.0296 | 4.92016 | 90.76 |
| 18.5983 | 4.77097 | 91.59 |
| 19.0688 | 4.65429 | 81.38 |
| 19.5621 | 4.53804 | 45.33 |
| 20.7297 | 4.28499 | 100.00 |
| 20.9634 | 4.23774 | 91.34 |
| 21.7966 | 4.07760 | 76.08 |
| 22.3214 | 3.98290 | 46.81 |
| 22.5311 | 3.94631 | 51.52 |
| 23.5818 | 3.77281 | 38.51 |
| 24.2623 | 3.66547 | 10.61 |
| 24.7116 | 3.60282 | 46.09 |
| 25.0476 | 3.55524 | 40.09 |
| 25.8672 | 3.44443 | 11.63 |
| 26.4270 | 3.37272 | 23.11 |
| 26.7575 | 3.32905 | 8.71 |
| 27.3139 | 3.26519 | 9.95 |
| 27.9165 | 3.19606 | 22.02 |
| 28.6990 | 3.11067 | 18.79 |
| 29.1752 | 3.06097 | 20.62 |
| 29.5761 | 3.02039 | 16.81 |
| 30.5469 | 2.92657 | 11.80 |
| 31.6182 | 2.82982 | 7.42 |
| 32.7077 | 2.73801 | 5.79 |
| 33.5815 | 2.66873 | 6.21 |

Table 23 below provides the results of the XRPD performed on sulfuric acid salt Pattern 3. The XRPD exhibited sharp peaks, indicating the sample was composed of crystalline material. Significant peaks were observed in the XRPD on sulfuric acid salt Pattern 3 at about 6.1±0.2°, about 13.6±0.2°, about 16.1±0.2°, about 17.6±0.2°, about 20.7±0.2°, about 22.7±0.2°, about 26.8±0.2°, about 26.9±0.2°, and about 27.8±0.2°. 5

TABLE 23
XRPD Peak List For Sulfuric Acid Salt Pattern 3

| Pos. [°2θ] | d-spacing [Å] | Rel. Int. [%] |
|---|---|---|
| 6.0597 | 14.58553 | 50.05 |
| 7.4423 | 11.87874 | 5.94 |
| 9.1049 | 9.71304 | 11.34 |
| 11.3656 | 7.78557 | 10.91 |
| 13.6374 | 6.49330 | 24.75 |
| 13.9813 | 6.33437 | 18.59 |
| 15.2489 | 5.81051 | 7.41 |
| 16.1378 | 5.49243 | 42.94 |
| 16.4319 | 5.39477 | 19.14 |
| 17.5588 | 5.05100 | 100.00 |
| 18.2847 | 4.85209 | 17.44 |
| 19.0974 | 4.64739 | 6.37 |
| 20.0509 | 4.42850 | 9.50 |
| 20.7162 | 4.28775 | 21.63 |
| 21.1925 | 4.19245 | 16.13 |
| 21.4105 | 4.15026 | 15.60 |
| 22.6738 | 3.91855 | 50.99 |
| 22.7397 | 3.91059 | 51.92 |
| 24.4743 | 3.63721 | 19.27 |
| 25.3115 | 3.51878 | 2.77 |
| 26.7736 | 3.32708 | 66.65 |
| 26.8590 | 3.32494 | 59.27 |
| 27.4818 | 3.24293 | 6.05 |
| 27.8407 | 3.20193 | 20.63 |
| 28.3537 | 3.14516 | 11.98 |
| 29.0109 | 3.07539 | 13.83 |
| 29.8031 | 2.99542 | 3.47 |
| 30.2888 | 2.94848 | 7.88 |
| 30.8140 | 2.89942 | 3.67 |
| 31.5490 | 2.83352 | 5.31 |
| 32.6581 | 2.73978 | 1.73 |
| 33.4162 | 2.67934 | 2.42 |

Table 24 below provides the results of the XRPD performed on sulfuric acid salt Pattern 4. The XRPD exhibited sharp peaks, indicating the sample was composed of crystalline material. Significant peaks were observed in the XRPD on sulfuric acid salt Pattern 4 at about 4.6±0.2°, 10 about 11.9±0.2°, about 12.5±0.2°, about 16.8±0.2°, about 17.8±0.2°, about 19.1±0.2°, about 20.3±0.2°, about 20.9±0.2°, about 21.1±0.2°, and about 23.0±0.2°.

TABLE 24
XRPD Peak List For Sulfuric Acid Salt Pattern 4

| Pos. [°2θ] | d-spacing [Å] | Rel. Int. [%] |
|---|---|---|
| 4.5855 | 19.27077 | 100.00 |
| 6.5625 | 13.46918 | 2.40 |
| 7.1291 | 12.39987 | 11.07 |
| 8.8203 | 10.02573 | 11.30 |
| 10.1496 | 8.71544 | 9.72 |
| 10.8944 | 8.12123 | 14.51 |
| 11.2872 | 7.83948 | 8.56 |
| 11.8580 | 7.46335 | 19.47 |
| 12.4900 | 7.08712 | 81.98 |
| 12.9359 | 6.84381 | 7.33 |
| 13.3262 | 6.64421 | 12.59 |
| 13.8055 | 6.41459 | 3.01 |
| 14.2753 | 6.20453 | 6.71 |
| 14.6037 | 6.06577 | 4.54 |
| 14.8820 | 5.95296 | 6.72 |
| 15.0836 | 5.87384 | 4.60 |
| 16.8204 | 5.27104 | 20.27 |
| 17.1863 | 5.15964 | 15.28 |
| 17.4595 | 5.07949 | 12.99 |
| 17.7931 | 4.98500 | 27.01 |
| 18.5055 | 4.79468 | 6.65 |
| 19.1307 | 4.63937 | 25.59 |
| 19.8282 | 4.47773 | 6.91 |
| 20.2616 | 4.38291 | 50.87 |
| 20.9085 | 4.24876 | 20.14 |
| 21.1421 | 4.20232 | 18.98 |
| 21.5826 | 4.11754 | 6.95 |
| 22.2616 | 3.99347 | 7.63 |
| 22.6253 | 3.93008 | 8.96 |
| 23.0466 | 3.85919 | 51.69 |
| 23.4460 | 3.79435 | 15.86 |
| 23.6104 | 3.76830 | 15.81 |
| 23.8598 | 3.72947 | 5.90 |
| 24.6990 | 3.60165 | 3.55 |
| 24.9506 | 3.56885 | 5.12 |
| 25.5892 | 3.48121 | 5.44 |
| 26.7355 | 3.33449 | 17.11 |
| 27.0487 | 3.29660 | 7.22 |
| 27.5685 | 3.23293 | 2.79 |
| 27.8492 | 3.20098 | 3.17 |
| 28.2591 | 3.15808 | 5.36 |
| 29.0978 | 3.06894 | 7.10 |
| 29.8860 | 2.98977 | 7.05 |
| 30.9024 | 2.89372 | 4.87 |
| 31.9343 | 2.80253 | 5.43 |
| 32.2676 | 2.77433 | 4.78 |
| 33.5937 | 2.66779 | 3.63 |
| 34.3311 | 2.61216 | 2.34 |

Table 25 below provides the results of the XRPD performed on sulfuric acid salt Pattern 5. The XRPD exhibited sharp peaks, indicating the sample was composed of crystalline material. Significant peaks were observed in the XRPD on sulfuric acid salt Pattern 5 at about 4.3±0.2°, about 4.6±0.2°, about 14.0±0.2°, about 17.6±0.2°, about 17.9±0.2°, about 19.4±0.2°, about 19.6±0.2°, about 20.0±0.2°, about 20.1±0.2°, about 22.7±0.2°, and about 23.7±0.2°.

TABLE 25
XRPD Peak List For Sulfuric Acid Salt Pattern 5

| Pos. [°2θ] | d-spacing [Å] | Rel. Int. [%] |
|---|---|---|
| 4.30 | 20.5498 | 43.41 |
| 4.56 | 19.3602 | 100.00 |
| 7.31 | 12.1007 | 9.77 |
| 9.49 | 9.3152 | 20.99 |
| 9.77 | 9.0545 | 11.20 |
| 11.26 | 7.8573 | 17.31 |
| 11.75 | 7.5329 | 7.62 |
| 11.94 | 7.4149 | 9.21 |
| 12.18 | 7.2688 | 8.19 |
| 12.97 | 6.8240 | 8.23 |
| 13.74 | 6.4455 | 15.30 |
| 14.04 | 6.3074 | 37.01 |
| 14.31 | 6.1878 | 19.63 |
| 14.96 | 5.9236 | 13.97 |
| 15.50 | 5.7130 | 3.83 |
| 15.91 | 5.5696 | 7.15 |
| 16.41 | 5.4021 | 17.42 |
| 16.81 | 5.2749 | 14.04 |
| 17.60 | 5.0388 | 32.52 |
| 17.86 | 4.9661 | 36.01 |
| 18.76 | 4.7292 | 8.34 |
| 19.07 | 4.6535 | 16.85 |
| 19.44 | 4.5665 | 25.06 |
| 19.61 | 4.5263 | 25.25 |
| 20.02 | 4.4305 | 26.27 |
| 20.10 | 4.4177 | 27.84 |
| 20.73 | 4.2858 | 13.19 |
| 21.28 | 4.1748 | 14.59 |
| 22.05 | 4.0319 | 8.60 |
| 22.66 | 3.9233 | 33.82 |
| 22.99 | 3.8678 | 8.72 |
| 23.71 | 3.7523 | 42.89 |
| 24.11 | 3.6888 | 6.83 |
| 24.68 | 3.6067 | 7.93 |
| 25.29 | 3.5221 | 18.44 |
| 25.59 | 3.4817 | 14.31 |
| 26.09 | 3.4150 | 18.20 |
| 26.45 | 3.3676 | 5.71 |
| 26.73 | 3.3329 | 4.58 |
| 27.88 | 3.1997 | 11.51 |
| 28.15 | 3.1698 | 14.56 |
| 28.57 | 3.1222 | 5.52 |
| 29.57 | 3.0206 | 3.74 |
| 30.30 | 2.9499 | 4.13 |
| 30.74 | 2.9083 | 3.86 |
| 31.97 | 2.7996 | 4.30 |
| 33.06 | 2.7093 | 2.90 |
| 34.18 | 2.6233 | 6.03 |

Table 26 below provides the results of the XRPD performed on sulfuric acid salt Pattern 6. Significant peaks were observed in the XRPD on sulfuric acid salt Pattern 6 at about 3.9±0.2°, about 11.8±0.2°, about 19.7±0.2°, and about 21.3±0.2°.

TABLE 26
XRPD Peak List For Sulfuric Acid Salt Pattern 6

| Pos. [°2θ] | d-spacing [Å] | Rel. Int. [%] |
|---|---|---|
| 3.9151 | 22.5688 | 100.00 |
| 10.5891 | 8.3547 | 4.39 |
| 11.7591 | 7.5260 | 58.16 |
| 15.8372 | 5.5960 | 5.85 |
| 18.2869 | 4.8515 | 8.41 |
| 19.7024 | 4.5060 | 34.88 |
| 21.3510 | 4.1617 | 30.59 |
| 25.1244 | 3.5446 | 7.08 |
| 27.7145 | 3.2189 | 6.03 |
| 29.0518 | 3.0737 | 1.90 |
| 30.7024 | 2.9121 | 1.77 |
| 31.5045 | 2.8398 | 1.42 |

Table 27 below provides the results of the XRPD performed on sulfuric acid salt Pattern 7. The XRPD exhibited sharp peaks, indicating the sample was composed of crystalline material. Significant peaks were observed in the XRPD on sulfuric acid salt Pattern 7 at about 4.1±0.2°, about 4.6±0.2°, about 14.0±0.2°, about 14.9±0.2°, about 16.3±0.2°, about 17.8±0.2°, about 10 19.3±0.2°, about 20.0±0.2°, about 23.7±0.2°, and about 26.00.2°.

TABLE 27
XRPD Peak List For Sulfuric Acid Salt Pattern 7

| Pos. [°2θ] | d-spacing [Å] | Rel. Int. [%] |
|---|---|---|
| 4.1179 | 21.4582 | 60.04 |
| 4.6368 | 19.0579 | 82.37 |
| 7.3113 | 12.0912 | 24.98 |
| 9.1154 | 9.7018 | 3.37 |
| 10.5867 | 8.3566 | 24.07 |
| 11.2134 | 7.8909 | 16.02 |
| 12.1371 | 7.2924 | 26.14 |
| 12.3841 | 7.1475 | 30.34 |
| 14.0363 | 6.3097 | 100.00 |
| 14.9317 | 5.9333 | 38.25 |
| 16.2891 | 5.4417 | 46.98 |
| 17.0126 | 5.2119 | 15.67 |
| 17.8517 | 4.9688 | 87.82 |
| 18.6691 | 4.7530 | 28.48 |
| 19.3210 | 4.5941 | 37.71 |
| 19.9819 | 4.4436 | 53.86 |
| 20.7126 | 4.2885 | 32.75 |
| 21.2567 | 4.1799 | 26.97 |
| 22.0334 | 4.0343 | 29.35 |
| 22.5658 | 3.9403 | 24.86 |
| 22.9888 | 3.8688 | 18.65 |
| 23.7206 | 3.7510 | 43.99 |
| 24.4474 | 3.6412 | 17.97 |
| 26.0290 | 3.4234 | 51.85 |
| 27.8496 | 3.2036 | 29.29 |
| 28.5008 | 3.1319 | 10.02 |
| 29.0621 | 3.0726 | 11.50 |
| 30.8355 | 2.8999 | 3.82 |
| 32.9583 | 2.7178 | 4.80 |

Table 28 below provides the results of the XRPD performed on methanesulfonic acid salt Pattern 1. The XRPD exhibited sharp peaks, indicating the sample was composed of crystalline material. Significant peaks were observed in the XRPD on methanesulfonic acid salt Pattern 1 at about 11.1±0.2°, about 15.8±0.2°, about 15.9±0.2°, about 16.6±0.2°, about 18.8±0.2°, about 21.0±0.2°, about 22.3±0.2°, and about 25.9±0.2°.

TABLE 28
XRPD Peak List For Methanesulfonic Acid Salt Pattern 1

| Pos. [°2θ] | d-spacing [Å] | Rel. Int. [%] |
|---|---|---|
| 7.2637 | 12.1704 | 3.72 |
| 8.2307 | 10.7425 | 1.26 |
| 10.1176 | 8.7430 | 3.74 |
| 10.2735 | 8.6106 | 1.69 |
| 11.1145 | 7.9609 | 100.00 |
| 11.5909 | 7.6348 | 3.15 |
| 11.8828 | 7.4479 | 4.51 |
| 13.6979 | 6.4648 | 5.30 |
| 14.3324 | 6.1800 | 3.50 |
| 14.5508 | 6.0877 | 5.43 |
| 15.3288 | 5.7804 | 3.23 |
| 15.8252 | 5.6002 | 11.69 |
| 15.9435 | 5.5589 | 11.21 |
| 16.6438 | 5.3266 | 14.38 |
| 17.1727 | 5.1637 | 2.20 |
| 18.8441 | 4.7093 | 8.95 |
| 19.5127 | 4.5494 | 2.60 |
| 19.8494 | 4.4730 | 4.57 |
| 20.0386 | 4.4312 | 2.99 |
| 20.2639 | 4.3824 | 1.51 |
| 21.0283 | 4.2248 | 20.07 |
| 21.3375 | 4.1643 | 5.21 |
| 21.5737 | 4.1192 | 2.63 |
| 21.9183 | 4.0552 | 1.30 |
| 22.3461 | 3.9786 | 9.94 |
| 23.0358 | 3.8610 | 4.71 |
| 23.3139 | 3.8156 | 4.85 |
| 23.9014 | 3.7231 | 1.40 |
| 24.3748 | 3.6518 | 2.47 |
| 24.8234 | 3.5868 | 2.09 |
| 25.2408 | 3.5285 | 3.40 |
| 25.4145 | 3.5047 | 3.56 |
| 25.6237 | 3.4766 | 7.55 |
| 25.8606 | 3.4453 | 23.19 |
| 26.1500 | 3.4078 | 2.23 |
| 27.3463 | 3.2614 | 3.47 |
| 27.6011 | 3.2319 | 2.96 |
| 28.0717 | 3.1787 | 0.96 |
| 28.5464 | 3.1270 | 0.78 |
| 29.5351 | 3.0245 | 5.43 |
| 29.9954 | 2.9791 | 4.75 |
| 30.3608 | 2.9441 | 3.77 |
| 31.0079 | 2.8841 | 4.09 |
| 32.0152 | 2.7956 | 4.73 |
| 33.2943 | 2.6911 | 1.08 |
| 33.8912 | 2.6451 | 1.84 |
| 34.6217 | 2.5909 | 0.73 |

Table 29 below provides the results of the XRPD performed on methanesulfonic acid salt Pattern 2. The XRPD exhibited sharp peaks, indicating the sample was composed of crystalline material. Significant peaks were observed in the XRPD on methanesulfonic acid salt Pattern 2 at about 6.7±0.2°, about 9.1±0.2°, about 13.4±0.2°, about 14.2±0.2°, about 18.1±0.2°, about 18.8±0.2°, about 19.0±0.2°, about 19.7±0.2°, about 20.1±0.2°, about 25.4±0.2°, and about 25.7±0.2°.

TABLE 29
XRPD Peak List For Methanesulfonic Acid Salt Pattern 2

| Pos. [°2θ] | d-spacing [Å] | Rel. Int. [%] |
|---|---|---|
| 6.6996 | 13.1939 | 56.58 |
| 8.5014 | 10.4011 | 29.31 |
| 9.1418 | 9.6739 | 99.17 |
| 9.5755 | 9.2366 | 12.37 |
| 11.2435 | 7.8698 | 14.17 |
| 11.9534 | 7.4040 | 10.79 |
| 13.4311 | 6.5926 | 49.89 |
| 13.8295 | 6.4035 | 26.32 |
| 14.2170 | 6.2298 | 38.28 |
| 16.1861 | 5.4761 | 6.70 |
| 16.8538 | 5.2607 | 13.67 |
| 17.2211 | 5.1493 | 29.50 |
| 17.6298 | 5.0308 | 25.98 |
| 18.1528 | 4.8870 | 32.33 |
| 18.8312 | 4.7125 | 54.97 |
| 19.0108 | 4.6684 | 100.00 |
| 19.7109 | 4.5041 | 42.06 |
| 20.0858 | 4.4209 | 30.44 |
| 20.2613 | 4.3830 | 23.22 |
| 20.5980 | 4.3121 | 21.00 |
| 21.6723 | 4.1007 | 7.19 |
| 22.1413 | 4.0116 | 3.71 |
| 22.3670 | 3.9749 | 9.78 |
| 22.6286 | 3.9295 | 11.98 |
| 23.0400 | 3.8603 | 17.61 |
| 23.4071 | 3.8006 | 10.05 |
| 23.6445 | 3.7630 | 6.65 |
| 24.3029 | 3.6625 | 5.90 |
| 24.6054 | 3.6151 | 3.18 |
| 25.0543 | 3.5543 | 9.61 |
| 25.4585 | 3.4988 | 30.45 |
| 25.6814 | 3.4689 | 34.08 |
| 26.3211 | 3.3861 | 8.70 |
| 26.9914 | 3.3035 | 11.27 |
| 28.0519 | 3.1809 | 5.13 |
| 28.7321 | 3.1072 | 9.62 |
| 29.0089 | 3.0782 | 27.09 |
| 29.9014 | 2.9883 | 6.58 |
| 30.2333 | 2.9562 | 3.42 |
| 31.0243 | 2.8826 | 6.30 |
| 32.0268 | 2.7947 | 4.63 |
| 32.7329 | 2.7360 | 2.22 |
| 33.1282 | 2.7042 | 2.05 |
| 34.0335 | 2.6343 | 3.78 |
| 34.4976 | 2.5999 | 2.38 |

Table 30 below provides the results of the XRPD performed on methanesulfonic acid salt Pattern 3. The XRPD exhibited sharp peaks, indicating the sample was composed of crystalline material. Significant peaks were observed in the XRPD on methanesulfonic acid salt Pattern 3 at about 7.4±0.2°, about 11.1±0.2°, about 12.2±0.2°, about 16.8±0.2°, about 17.5±0.2°, about 19.5±0.2°, about 22.0±0.2°, about 24.7±0.2°, about 25.8±0.2°, and about 26.5±0.2°.

TABLE 30
XRPD Peak List For Methanesulfonic Acid Salt Pattern 3

| Pos. [°2θ] | d-spacing [Å] | Rel. Int. [%] |
|---|---|---|
| 3.2904 | 26.852 | 2.55 |
| 7.4153 | 11.922 | 100.00 |
| 8.1705 | 10.822 | 14.71 |
| 8.4207 | 10.501 | 6.15 |
| 10.1181 | 8.743 | 3.08 |
| 11.1157 | 7.960 | 57.24 |
| 11.6052 | 7.625 | 5.74 |
| 12.2221 | 7.242 | 39.98 |
| 13.1732 | 6.721 | 17.58 |
| 13.6959 | 6.466 | 4.58 |
| 14.3126 | 6.183 | 4.15 |
| 14.8745 | 5.956 | 15.57 |
| 15.8384 | 5.596 | 10.92 |
| 16.4153 | 5.400 | 17.61 |
| 16.8018 | 5.277 | 72.36 |
| 17.5024 | 5.067 | 35.83 |
| 18.8411 | 4.710 | 25.17 |
| 19.1893 | 4.625 | 20.45 |
| 19.4918 | 4.554 | 36.83 |
| 19.8569 | 4.471 | 16.73 |
| 20.1679 | 4.403 | 19.19 |
| 21.0590 | 4.219 | 17.82 |
| 21.9629 | 4.047 | 33.60 |
| 22.3634 | 3.976 | 16.85 |
| 23.4229 | 3.798 | 12.58 |
| 23.6799 | 3.757 | 13.06 |
| 24.2233 | 3.674 | 17.57 |
| 24.7331 | 3.600 | 33.57 |
| 25.2191 | 3.531 | 19.24 |
| 25.8241 | 3.450 | 28.25 |
| 26.5373 | 3.359 | 30.36 |
| 26.9458 | 3.309 | 5.18 |
| 27.3501 | 3.258 | 4.51 |
| 28.1743 | 3.167 | 8.72 |
| 28.7656 | 3.104 | 3.85 |
| 29.2946 | 3.049 | 5.69 |
| 30.1637 | 2.963 | 10.19 |
| 31.6008 | 2.831 | 2.83 |
| 32.0363 | 2.794 | 6.43 |
| 33.1875 | 2.700 | 4.96 |
| 34.2963 | 2.615 | 2.34 |

Table 31 below provides the results of the XRPD performed on methanesulfonic acid salt Pattern 4. The XRPD exhibited sharp peaks, indicating the sample was composed of crystalline material. Significant peaks were observed in the XRPD on methanesulfonic acid salt Pattern 4 at about 10.4±0.2°, about 11.6±0.2°, about 12.3±0.2°, about 16.1±0.2°, about 18.0±0.2°, about 20.1±0.2°, about 20.9±0.2°, about 21.4±0.2°, about 22.5±0.2°, and about 22.6±0.2°.

TABLE 31
XRPD Peak List For Methanesulfonic Acid Salt Pattern 4

| Pos. [°2θ] | d-spacing [Å] | Rel. Int. [%] |
|---|---|---|
| 7.6632 | 11.5368 | 0.95 |
| 10.3612 | 8.5380 | 100.00 |
| 11.0810 | 7.9849 | 10.45 |
| 11.5651 | 7.6517 | 68.67 |
| 12.3395 | 7.1732 | 15.54 |
| 14.6037 | 6.0658 | 4.94 |
| 14.9565 | 5.9235 | 3.30 |
| 15.4260 | 5.7442 | 8.58 |
| 16.1509 | 5.4880 | 14.53 |
| 16.4663 | 5.3836 | 4.29 |
| 17.4360 | 5.0863 | 3.24 |
| 17.9753 | 4.9349 | 20.10 |
| 18.3531 | 4.8341 | 13.05 |
| 18.8079 | 4.7183 | 3.54 |
| 19.4373 | 4.5669 | 14.32 |
| 20.1069 | 4.4163 | 30.23 |
| 20.3629 | 4.3613 | 9.07 |
| 20.9176 | 4.2469 | 19.35 |
| 21.4212 | 4.1482 | 33.78 |
| 22.2437 | 3.9967 | 10.38 |
| 22.5366 | 3.9421 | 35.34 |
| 22.5897 | 3.9362 | 37.59 |
| 23.1156 | 3.8478 | 9.84 |
| 23.6411 | 3.7604 | 1.81 |
| 24.0338 | 3.7029 | 2.96 |
| 24.6247 | 3.6153 | 11.39 |
| 24.8311 | 3.5857 | 9.99 |
| 25.0615 | 3.5533 | 5.03 |
| 26.1275 | 3.4107 | 7.95 |
| 26.4898 | 3.3649 | 4.58 |
| 26.9109 | 3.3132 | 5.69 |
| 27.4749 | 3.2464 | 4.36 |
| 27.9885 | 3.1880 | 8.54 |
| 28.7477 | 3.1055 | 12.05 |
| 29.2286 | 3.0555 | 7.81 |
| 29.5539 | 3.0226 | 7.70 |
| 31.4000 | 2.8490 | 1.72 |
| 31.9148 | 2.8042 | 3.96 |
| 32.4162 | 2.7620 | 2.79 |
| 32.9141 | 2.7213 | 3.62 |
| 34.0500 | 2.6331 | 2.30 |

Table 32 below provides the results of the XRPD performed on methanesulfonic acid salt Pattern 5. The XRPD exhibited sharp peaks, indicating the sample was composed of crystalline material. Significant peaks were observed in the XRPD on methanesulfonic acid salt Pattern 5 at about 10.6±0.2°, about 11.1±0.2°, about 11.9±0.2°, about 17.4±0.2°, about 17.6±0.2°, about 20.6±0.2°, about 21.1±0.2°, about 23.1±0.2°, about 24.0±0.2°, and about 25.8±0.2°.

TABLE 32
XRPD Peak List For Methanesulfonic Acid Salt Pattern 5

| Pos. [°2θ] | d-spacing [Å] | Rel. Int. [%] |
|---|---|---|
| 6.5462 | 13.5026 | 18.99 |
| 7.2501 | 12.1931 | 3.75 |
| 8.1935 | 10.7913 | 2.77 |
| 8.4774 | 10.4305 | 3.95 |
| 9.0848 | 9.7345 | 21.04 |
| 10.5822 | 8.3601 | 27.98 |
| 11.1075 | 7.9659 | 86.36 |
| 11.9215 | 7.4238 | 27.06 |
| 12.9907 | 6.8151 | 25.20 |
| 14.2825 | 6.2014 | 7.38 |
| 15.1145 | 5.8619 | 21.60 |
| 15.8737 | 5.5832 | 18.03 |
| 16.2100 | 5.4681 | 22.77 |
| 16.6114 | 5.3369 | 22.96 |
| 17.4345 | 5.0867 | 62.48 |
| 17.6481 | 5.0257 | 37.83 |
| 18.5106 | 4.7934 | 15.41 |
| 18.8836 | 4.6995 | 19.29 |
| 19.7790 | 4.4888 | 10.40 |
| 20.5984 | 4.3120 | 29.04 |
| 21.0648 | 4.2176 | 51.44 |
| 21.6034 | 4.1136 | 9.67 |
| 22.2956 | 3.9875 | 14.05 |
| 23.0614 | 3.8568 | 100.00 |
| 24.0233 | 3.7045 | 27.37 |
| 24.8143 | 3.5881 | 5.56 |
| 25.8510 | 3.4466 | 46.21 |
| 26.6179 | 3.3490 | 14.66 |
| 27.5487 | 3.2379 | 10.24 |
| 28.2076 | 3.1637 | 23.32 |
| 29.0151 | 3.0775 | 8.89 |
| 29.5826 | 3.0197 | 9.61 |
| 30.4338 | 2.9372 | 6.81 |
| 31.1956 | 2.8672 | 6.03 |
| 32.0690 | 2.7911 | 6.96 |
| 32.6837 | 2.7377 | 3.06 |
| 33.8874 | 2.6453 | 5.82 |

Table 33 below provides the results of the XRPD performed on maleic acid salt Pattern 1. The XRPD exhibited sharp peaks, indicating the sample was composed of crystalline material. Significant peaks were observed in the XRPD on maleic acid salt Pattern 1 at about 5.5±0.2°, about 10.9±0.2°, about 14.0±0.2°, about 17.3±0.2°, about 17.7±0.2°, about 18.0±0.2°, about 18.1±0.2°, about 26.9±0.2°, about 27.7±0.2°, and about 29.0±0.2°.

TABLE 33
XRPD Peak List For Maleic Acid Salt Pattern 1

| Pos. [°2θ] | d-spacing [Å] | Rel. Int. [%] |
|---|---|---|
| 5.5423 | 15.94591 | 64.37 |
| 7.1273 | 12.40306 | 11.76 |
| 7.6553 | 11.54868 | 18.59 |
| 9.0101 | 9.81499 | 2.83 |
| 10.9492 | 8.08072 | 33.01 |
| 13.1829 | 6.71613 | 8.70 |
| 14.0217 | 6.31620 | 24.30 |
| 14.5597 | 6.08400 | 15.42 |
| 14.8240 | 5.97609 | 13.22 |
| 15.6174 | 5.67423 | 8.90 |
| 16.0621 | 5.51814 | 18.06 |
| 16.6825 | 5.31431 | 6.79 |
| 17.2998 | 5.12603 | 23.57 |
| 17.7447 | 4.99849 | 25.88 |
| 17.9902 | 4.93084 | 23.61 |
| 18.1443 | 4.88930 | 21.16 |
| 19.0270 | 4.66442 | 6.87 |
| 19.5735 | 4.53542 | 11.10 |
| 20.4043 | 4.35259 | 7.00 |
| 20.7697 | 4.27683 | 5.38 |
| 21.6800 | 4.09927 | 8.18 |
| 22.0268 | 4.03551 | 6.61 |
| 22.2817 | 3.98992 | 7.38 |
| 22.5033 | 3.95112 | 5.80 |
| 23.0585 | 3.85723 | 5.80 |
| 23.9163 | 3.72079 | 6.25 |
| 24.2640 | 3.66825 | 3.91 |
| 24.9902 | 3.56328 | 2.31 |
| 26.0650 | 3.41874 | 4.39 |
| 26.8885 | 3.31587 | 100.00 |
| 27.3159 | 3.26495 | 7.77 |
| 27.7412 | 3.21585 | 33.81 |
| 28.9595 | 3.08328 | 24.37 |
| 29.7669 | 3.00146 | 5.15 |
| 30.4758 | 2.93324 | 9.63 |
| 31.6889 | 2.82367 | 2.13 |
| 32.0919 | 2.78912 | 2.69 |
| 32.5254 | 2.75294 | 1.90 |
| 33.7640 | 2.65473 | 3.25 |

Table 34 below provides the results of the XRPD performed on maleic acid salt Pattern 1*. (* indicates slight shifts in XRPD peak positions in comparison with maleic Acid Pattern 1.) The XRPD exhibited sharp peaks, indicating the sample was composed of crystalline material. Significant peaks were observed in the XRPD on maleic acid salt Pattern 1* at about 5.5±0.2°, about 7.1±0.2°, about 7.7±0.2°, about 11.0±0.2°, about 14.6±0.2°, about 17.7±0.2°, about 18.2±0.2°, about 26.9±0.2°, about 27.8±0.2°, and about 29.1±0.2°.

TABLE 34
XRPD Peak List For Maleic Acid Salt Pattern 1*

| Pos. [°2θ] | d-spacing [Å] | Rel. Int. [%] |
|---|---|---|
| 5.5503 | 15.92311 | 95.08 |
| 7.1389 | 12.38278 | 40.47 |
| 7.6701 | 11.52647 | 56.62 |
| 9.0369 | 9.78598 | 5.80 |
| 10.6097 | 8.33855 | 30.46 |
| 10.9774 | 8.06004 | 62.96 |
| 13.2517 | 6.68142 | 16.30 |
| 13.4335 | 6.59138 | 9.54 |
| 14.0592 | 6.29941 | 30.27 |
| 14.5767 | 6.07693 | 37.53 |
| 14.8308 | 5.97338 | 17.63 |
| 15.6593 | 5.65915 | 14.99 |
| 16.0949 | 5.50696 | 21.30 |
| 16.7324 | 5.29856 | 9.45 |
| 17.2612 | 5.13740 | 28.58 |
| 17.7441 | 4.99867 | 34.75 |
| 18.1831 | 4.87897 | 36.56 |
| 18.9909 | 4.67323 | 13.53 |
| 19.3534 | 4.58649 | 14.10 |
| 19.6716 | 4.51303 | 17.26 |
| 20.4552 | 4.34187 | 9.99 |
| 20.7842 | 4.27388 | 12.33 |
| 21.7430 | 4.08753 | 22.19 |
| 22.3393 | 3.97975 | 17.97 |
| 22.6941 | 3.91833 | 15.40 |
| 23.1588 | 3.84076 | 10.03 |
| 23.8694 | 3.72800 | 10.13 |
| 24.3307 | 3.65834 | 8.27 |
| 24.9186 | 3.57335 | 4.34 |
| 25.8219 | 3.45036 | 8.00 |
| 26.9462 | 3.30891 | 100.00 |
| 27.4125 | 3.25097 | 13.76 |
| 27.8175 | 3.20721 | 39.10 |
| 29.0704 | 3.07177 | 36.87 |
| 29.7729 | 3.00087 | 9.43 |
| 30.6461 | 2.91733 | 9.50 |
| 31.7629 | 2.81726 | 4.69 |
| 32.1556 | 2.78375 | 6.40 |
| 33.3772 | 2.68460 | 4.00 |
| 33.8091 | 2.65129 | 4.47 |

Table 35 below provides the results of the XRPD performed on maleic acid salt Pattern 2. The XRPD exhibited sharp peaks, indicating the sample was composed of crystalline material. Significant peaks were observed in the XRPD on maleic acid salt Pattern 2 at about 4.4±0.2°, about 8.9±0.2°, about 9.7±0.2°, about 10.3±0.2°, about 16.0±0.2°, about 16.9±0.2°, about 17.1±0.2°. about 19.7±0.2°, about 22.3±0.2°, and about 24.5±0.2°.

TABLE 35
XRPD Peak List For Maleic Acid Salt Pattern 2

| Pos. [°2θ] | d-spacing [Å] | Rel. Int. [%] |
|---|---|---|
| 4.4294 | 19.94971 | 58.23 |
| 4.8737 | 18.13185 | 10.13 |
| 7.6731 | 11.52196 | 5.69 |
| 7.9200 | 11.16327 | 10.81 |
| 8.8626 | 9.97804 | 44.34 |
| 9.7442 | 9.07712 | 22.31 |
| 10.2884 | 8.59818 | 59.90 |
| 11.9408 | 7.41183 | 5.58 |
| 13.0572 | 6.78052 | 14.94 |
| 13.7511 | 6.43452 | 1.64 |
| 15.2967 | 5.79249 | 16.59 |
| 15.8701 | 5.58447 | 15.87 |
| 16.0389 | 5.52607 | 22.97 |
| 16.9100 | 5.24331 | 28.85 |
| 17.0695 | 5.19468 | 51.17 |
| 17.9218 | 4.94950 | 6.72 |
| 18.6679 | 4.75335 | 7.51 |
| 19.6724 | 4.51284 | 100.00 |
| 20.0951 | 4.41886 | 17.38 |
| 20.6748 | 4.29625 | 17.02 |
| 21.3574 | 4.16045 | 11.62 |
| 21.7125 | 4.09321 | 12.45 |
| 22.2886 | 3.98869 | 27.03 |
| 22.5823 | 3.93747 | 18.93 |
| 22.8206 | 3.89690 | 8.34 |
| 23.9811 | 3.71088 | 10.92 |
| 24.5440 | 3.62704 | 29.94 |
| 25.4451 | 3.50060 | 5.31 |
| 25.7660 | 3.45773 | 5.12 |
| 26.3269 | 3.38532 | 8.70 |
| 27.0777 | 3.29313 | 5.72 |
| 27.4118 | 3.25375 | 6.25 |
| 27.7824 | 3.21118 | 5.86 |
| 28.2505 | 3.15902 | 3.56 |
| 28.8276 | 3.09709 | 2.47 |
| 29.7560 | 3.00254 | 5.60 |
| 30.2190 | 2.95758 | 7.25 |
| 30.9223 | 2.89190 | 2.68 |
| 32.2334 | 2.77720 | 1.25 |
| 33.6560 | 2.66300 | 4.58 |

Table 36 below provides the results of the XRPD performed on maleic acid salt Pattern 3. The XRPD exhibited sharp peaks, indicating the sample was composed of crystalline material. Significant peaks were observed in the XRPD on maleic acid salt Pattern 3 at about 5.4±0.2°, about 7.6±0.2°, about 10.8±0.2°, about 14.5±0.2°, about 14.8±0.2°, about 17.4±0.2°, about 26.4±0.2°, about 26.5±0.2°, about 27.2±0.2°, and about 27.6±0.2°.

TABLE 36
XRPD Peak List For Maleic Acid Salt Pattern 3

| Pos. [°2θ] | d-spacing [Å] | Rel. Int. [%] |
|---|---|---|
| 5.4545 | 16.2025 | 30.64 |
| 7.0872 | 12.4732 | 17.08 |
| 7.5917 | 11.6453 | 22.58 |
| 8.9461 | 9.8850 | 1.91 |
| 10.5652 | 8.3736 | 14.87 |
| 10.8256 | 8.1727 | 18.30 |
| 13.0783 | 6.7696 | 3.68 |
| 13.6940 | 6.4666 | 17.06 |
| 13.9777 | 6.3360 | 6.90 |
| 14.4807 | 6.1170 | 22.18 |
| 14.7699 | 5.9979 | 24.99 |
| 15.7582 | 5.6239 | 13.94 |
| 15.9861 | 5.5442 | 13.81 |
| 16.3992 | 5.4055 | 3.61 |
| 17.4263 | 5.0891 | 32.88 |
| 18.0999 | 4.9012 | 16.16 |
| 18.5545 | 4.7782 | 1.43 |
| 19.1854 | 4.6263 | 11.24 |
| 19.4295 | 4.5687 | 16.80 |
| 20.4411 | 4.3448 | 3.86 |
| 20.7350 | 4.2839 | 3.77 |
| 21.2372 | 4.1837 | 4.05 |
| 21.5683 | 4.1202 | 8.77 |
| 22.0790 | 4.0228 | 2.75 |
| 22.4273 | 3.9643 | 4.92 |
| 22.7339 | 3.9116 | 7.48 |
| 23.3013 | 3.8176 | 3.52 |
| 23.8841 | 3.7257 | 5.13 |
| 25.4209 | 3.5039 | 2.53 |
| 25.8407 | 3.4479 | 3.76 |
| 26.3601 | 3.3811 | 56.48 |
| 26.5492 | 3.3575 | 100.00 |
| 27.1648 | 3.2828 | 24.39 |
| 27.6441 | 3.2269 | 23.23 |
| 28.3296 | 3.1504 | 10.14 |
| 28.6436 | 3.1166 | 9.92 |
| 29.2881 | 3.0494 | 7.13 |
| 29.6856 | 3.0095 | 8.17 |
| 31.7505 | 2.8183 | 1.99 |
| 32.5906 | 2.7476 | 1.23 |
| 33.2311 | 2.6961 | 0.80 |

Table 37 below provides the results of the XRPD performed on maleic acid salt Pattern 3*. (* indicates slight shifts in XRPD peak positions in comparison with maleic Acid Pattern 3.) The XRPD exhibited sharp peaks, indicating the sample was composed of crystalline material. Significant peaks were observed in the XRPD on maleic acid salt Pattern 3* at about 5.5±0.2°, about 7.1±0.2°, about 7.6±0.2°, about 10.6±0.2°, about 11.0±0.2°, about 14.5±0.2°, about 16.1±0.2°, about 17.7±0.2°, about 18.2±0.2°, about 26.9±0.2°, and about 27.7±0.2°.

TABLE 37
XRPD Peak List For Maleic Acid Salt Pattern 3*

| Pos. [°2θ] | d-spacing [Å] | Rel. Int. [%] |
|---|---|---|
| 5.5294 | 15.98325 | 100.00 |
| 7.1178 | 12.41946 | 47.82 |
| 7.6241 | 11.59593 | 71.45 |
| 8.9580 | 9.87193 | 5.54 |
| 10.5749 | 8.36590 | 36.54 |
| 10.9716 | 8.06427 | 58.25 |
| 13.2180 | 6.69837 | 15.41 |
| 14.0123 | 6.32041 | 29.51 |
| 14.5498 | 6.08810 | 43.52 |
| 14.8349 | 5.97173 | 32.87 |
| 16.0723 | 5.51464 | 36.99 |
| 17.2754 | 5.13321 | 29.43 |
| 17.6885 | 5.01425 | 45.87 |
| 18.1569 | 4.88595 | 40.81 |
| 19.0029 | 4.67029 | 13.32 |
| 19.4238 | 4.57003 | 29.79 |
| 19.6439 | 4.51933 | 20.25 |
| 20.4290 | 4.34737 | 12.19 |
| 20.7604 | 4.27872 | 12.83 |
| 21.7275 | 4.09041 | 23.08 |
| 22.7009 | 3.91717 | 17.82 |
| 23.9518 | 3.71536 | 18.33 |
| 25.8463 | 3.44716 | 10.67 |
| 26.8688 | 3.31826 | 93.93 |
| 27.7266 | 3.21751 | 36.36 |
| 29.0782 | 3.07097 | 33.20 |
| 29.8218 | 2.99607 | 12.19 |
| 31.7736 | 2.81634 | 3.72 |

Table 38 below provides the results of the XRPD performed on maleic acid salt Pattern 4. The XRPD exhibited sharp peaks, indicating the sample was composed of crystalline material. Significant peaks were observed in the XRPD on maleic acid salt Pattern 4 at about 6.2±0.2°, about 8.7±0.2°, about 10.6±0.2°, about 12.5±0.2°, about 15.9±0.2°, about 16.1±0.2°, about 16.7±0.2°, 10 about 20.1±0.2°, about 25.8±0.2°, about 26.8±0.2°, and about 29.1±0.2°.

TABLE 38
XRPD Peak List For Maleic Acid Salt Pattern 4

| Pos. [°2θ] | d-spacing [Å] | Rel. Int. [%] |
|---|---|---|
| 5.1182 | 17.26632 | 18.45 |
| 6.2223 | 14.20474 | 52.49 |
| 7.0959 | 12.45785 | 16.30 |
| 8.7494 | 10.10684 | 53.25 |
| 9.6364 | 9.17845 | 4.58 |
| 10.2771 | 8.60762 | 24.93 |
| 10.5921 | 8.35238 | 74.23 |
| 10.8669 | 8.14174 | 17.45 |
| 12.4849 | 7.09001 | 71.10 |
| 13.4001 | 6.60777 | 26.40 |
| 13.8700 | 6.38495 | 41.43 |
| 14.2124 | 6.23186 | 24.89 |
| 14.6629 | 6.04138 | 37.21 |
| 15.4516 | 5.73476 | 47.83 |
| 15.8745 | 5.58290 | 57.12 |
| 16.1505 | 5.48812 | 75.57 |
| 16.7128 | 5.30472 | 51.61 |
| 17.2911 | 5.12858 | 48.94 |
| 17.7872 | 4.98666 | 40.70 |
| 18.5734 | 4.77730 | 47.01 |
| 18.8344 | 4.71169 | 45.31 |
| 19.3496 | 4.58738 | 35.57 |
| 19.6560 | 4.51657 | 32.14 |
| 20.1416 | 4.40876 | 52.40 |
| 20.6090 | 4.30982 | 49.07 |
| 21.3056 | 4.17045 | 29.14 |
| 21.6735 | 4.09709 | 14.25 |
| 22.3318 | 3.98107 | 31.33 |
| 22.6956 | 3.91807 | 32.50 |
| 23.8862 | 3.72542 | 20.73 |
| 24.4377 | 3.64257 | 16.64 |
| 25.7854 | 3.45517 | 75.74 |
| 26.7948 | 3.32725 | 100.00 |
| 28.0373 | 3.18256 | 25.13 |
| 29.0680 | 3.07202 | 50.43 |
| 31.1201 | 2.87396 | 10.56 |
| 32.7270 | 2.73643 | 4.51 |

Table 39 below provides the results of the XRPD performed on maleic acid salt Pattern 5. The XRPD exhibited sharp peaks, indicating the sample was composed of crystalline material. Significant peaks were observed in the XRPD on maleic acid salt Pattern 5 at about 5.1±0.2°, about 5.5±0.2°, about 7.1±0.2°, about 7.5±0.2°, about 10.6±0.2°, about 14.3±0.2°, about 17.5±0.2°, about 18.6±0.2°, about 25.8±0.2°, and about 26.9±0.2°.

TABLE 39
XRPD Peak List For Maleic Acid Salt Pattern 5

| Pos. [°2θ] | d-spacing [Å] | Rel. Int. [%] |
|---|---|---|
| 5.1346 | 17.2111 | 100.00 |
| 5.5249 | 15.9962 | 37.49 |
| 7.0602 | 12.5208 | 62.62 |
| 7.5011 | 11.7858 | 46.36 |
| 7.6457 | 11.5632 | 25.10 |
| 8.7216 | 10.1390 | 5.81 |
| 9.6321 | 9.1826 | 4.14 |
| 10.5838 | 8.3589 | 49.80 |
| 10.9391 | 8.0882 | 31.06 |
| 12.4646 | 7.1015 | 9.59 |
| 13.4019 | 6.6069 | 12.73 |
| 14.3375 | 6.1778 | 43.94 |
| 15.4382 | 5.7397 | 12.26 |
| 16.9590 | 5.2283 | 19.18 |
| 17.4633 | 5.0784 | 50.46 |
| 18.5737 | 4.7772 | 45.78 |
| 19.3181 | 4.5948 | 24.46 |
| 19.6462 | 4.5188 | 17.26 |
| 20.7202 | 4.2869 | 15.84 |
| 21.2318 | 4.1848 | 19.13 |
| 21.6844 | 4.0985 | 16.72 |
| 22.1554 | 4.0124 | 15.88 |
| 22.6533 | 3.9253 | 22.69 |
| 23.0487 | 3.8588 | 12.79 |
| 24.2645 | 3.6682 | 7.85 |
| 25.8164 | 3.4511 | 37.44 |
| 26.8748 | 3.3175 | 42.49 |
| 27.8040 | 3.2087 | 15.44 |
| 29.0678 | 3.0720 | 30.71 |
| 29.8561 | 2.9927 | 8.89 |
| 32.0175 | 2.7954 | 2.04 |

Table 40 below provides the results of the XRPD performed on phosphoric acid salt Pattern 1. The XRPD exhibited sharp peaks, indicating the sample was composed of crystalline material. Significant peaks were observed in the XRPD on phosphoric acid salt Pattern 1 at about 4.7±0.2°, about 13.5±0.2°, about 14.2±0.2°, about 17.3±0.2°, about 18.1±0.2°, about 18.9±0.2°, about 20.9±0.2°, about 21.2±0.2°, about 22.5±0.2°, about 23.2±0.2°, and about 24.0±0.2°.

TABLE 40
XRPD Peak List For Phosphoric Acid Salt Pattern 1

| Pos. [°2θ] | d-spacing [Å] | Rel. Int. [%] |
|---|---|---|
| 4.7108 | 18.75861 | 72.03 |
| 6.0574 | 14.59118 | 10.45 |
| 6.7449 | 13.10526 | 51.45 |
| 8.5345 | 10.36087 | 31.82 |
| 9.6925 | 9.12539 | 12.39 |
| 11.9766 | 7.38973 | 23.12 |
| 13.5439 | 6.53790 | 96.74 |
| 14.1594 | 6.25506 | 71.66 |
| 14.8475 | 5.96668 | 26.61 |
| 16.2589 | 5.45177 | 24.40 |
| 17.3069 | 5.12395 | 69.50 |
| 18.0703 | 4.90918 | 59.25 |
| 18.8774 | 4.70105 | 66.23 |
| 19.5784 | 4.53429 | 50.12 |
| 20.8661 | 4.25728 | 92.93 |
| 21.1610 | 4.19862 | 61.02 |
| 21.6748 | 4.10023 | 45.08 |
| 22.5259 | 3.94721 | 100.00 |
| 23.1921 | 3.83531 | 55.81 |
| 24.0430 | 3.70147 | 73.54 |
| 24.6878 | 3.60624 | 45.08 |
| 25.0430 | 3.55589 | 31.50 |
| 25.9314 | 3.43605 | 26.22 |
| 27.4000 | 3.25513 | 30.52 |
| 28.3530 | 3.14784 | 17.08 |
| 30.0007 | 2.97861 | 17.16 |
| 30.9025 | 2.89371 | 9.00 |

Table 41 below provides the results of the XRPD performed on phosphoric acid salt Pattern 2. Significant peaks were observed in the XRPD on phosphoric acid salt Pattern 2 at about 3.9±0.2°, about 7.1±0.2°, about 14.4±0.2°, about 16.1±0.2°, about 19.2±0.2°, about 20.8±0.2°, about 21.5±0.2°, about 23.2±0.2°, and about 24.0±0.2°.

TABLE 41
XRPD Peak List For Phosphoric Acid Salt Pattern 2

| Pos. [°2θ] | d-spacing [Å] | Rel. Int. [%] |
|---|---|---|
| 3.8783 | 22.7829 | 100.00 |
| 5.0715 | 17.4250 | 3.54 |
| 5.9035 | 14.9710 | 1.31 |
| 6.7190 | 13.1559 | 8.20 |
| 7.0730 | 12.4980 | 13.70 |
| 7.7492 | 11.4090 | 3.04 |
| 8.3760 | 10.5566 | 1.23 |
| 9.2594 | 9.5513 | 8.59 |
| 10.7300 | 8.2453 | 3.37 |
| 11.2075 | 7.8951 | 3.66 |
| 12.2870 | 7.2038 | 5.42 |
| 13.4853 | 6.5662 | 4.36 |
| 14.4113 | 6.1463 | 16.67 |
| 16.0916 | 5.5081 | 10.14 |
| 16.6131 | 5.3364 | 6.63 |
| 18.1384 | 4.8909 | 5.85 |
| 19.2373 | 4.6139 | 11.84 |
| 19.9432 | 4.4522 | 7.54 |
| 20.7831 | 4.2741 | 10.60 |
| 21.5160 | 4.1301 | 12.35 |
| 23.2294 | 3.8292 | 13.41 |
| 23.9872 | 3.7100 | 11.57 |
| 24.9958 | 3.5625 | 6.55 |
| 25.9090 | 3.4390 | 7.15 |
| 27.7902 | 3.2103 | 3.39 |
| 33.9662 | 2.6394 | 1.14 |

Table 42 below provides the results of the XRPD performed on phosphoric acid salt Pattern 3. The XRPD exhibited sharp peaks, indicating the sample was composed of crystalline material. Significant peaks were observed in the XRPD on phosphoric acid salt Pattern 3 at about 3.9±0.2°, about 10.2±0.2°, about 10.6±0.2°, about 13.4±0.2°, about 14.2±0.2°, about 19.6±0.2°, about 20.4±0.2°, about 21.1±0.2°, about 22.5±0.2°, and about 23.2±0.2°.

TABLE 42
XRPD Peak List For Phosphoric Acid Salt Pattern 3

| Pos. [°2θ] | d-spacing [Å] | Rel. Int. [%] |
|---|---|---|
| 3.8973 | 22.67181 | 100.0000 |
| 5.6832 | 15.55104 | 1.8600 |
| 6.7496 | 13.09612 | 1.8600 |
| 7.9258 | 11.15515 | 5.7900 |
| 10.1786 | 8.69068 | 24.4300 |
| 10.5888 | 8.35491 | 22.0700 |
| 11.7209 | 7.55035 | 3.8700 |
| 12.7380 | 6.94968 | 7.4400 |
| 13.3579 | 6.62853 | 16.0800 |
| 13.8665 | 6.38655 | 13.4900 |
| 14.2354 | 6.22183 | 32.5600 |
| 14.5881 | 6.07221 | 6.6700 |
| 15.1940 | 5.83140 | 8.8700 |
| 15.6199 | 5.67332 | 15.3700 |
| 16.1608 | 5.48467 | 10.5400 |
| 16.8510 | 5.26152 | 15.1600 |
| 17.4192 | 5.09116 | 10.1500 |
| 18.2699 | 4.85598 | 9.3900 |
| 19.6165 | 4.52556 | 26.1900 |
| 20.3684 | 4.36017 | 29.3900 |
| 21.0834 | 4.21390 | 26.9900 |
| 21.4607 | 4.14067 | 10.2600 |
| 21.7866 | 4.07945 | 11.4900 |
| 22.4895 | 3.95352 | 24.4300 |
| 23.2309 | 3.82900 | 85.1700 |
| 23.8089 | 3.73733 | 11.8400 |
| 24.3446 | 3.65630 | 8.6700 |
| 25.1630 | 3.53920 | 13.5400 |
| 25.8940 | 3.44092 | 7.0600 |
| 26.9197 | 3.31210 | 6.8500 |
| 27.6150 | 3.23026 | 9.6500 |
| 28.2978 | 3.15386 | 8.2300 |
| 29.1233 | 3.06631 | 7.6000 |
| 29.9125 | 2.98718 | 5.6900 |
| 31.8946 | 2.80593 | 3.4500 |
| 33.2443 | 2.69503 | 4.4800 |
| 34.1928 | 2.62241 | 4.0100 |

Table 43 below provides the results of the XRPD performed on phosphoric acid salt Pattern 4. Significant peaks were observed in the XRPD on phosphoric acid salt Pattern 4 at about 3.8±0.2°, about 9.9±0.2°, about 10.6±0.2°, about 13.4±0.2°, about 19.3±0.2°, about 19.6±0.2°, about 21.7±0.2°, and about 22.7±0.2°.

TABLE 43
XRPD Peak List For Phosphoric Acid Salt Pattern 4

| Pos. [°2θ] | d-spacing [Å] | Rel. Int. [%] |
|---|---|---|
| 3.8231 | 23.11217 | 100.0 |
| 5.3822 | 16.42003 | 2.1 |
| 6.6531 | 13.28589 | 4.0 |
| 7.6325 | 11.58309 | 4.7 |
| 9.8749 | 8.95733 | 10.4 |
| 10.5977 | 8.34795 | 17.8 |
| 12.5240 | 7.06797 | 3.1 |
| 13.4032 | 6.60622 | 13.7 |
| 14.9136 | 5.94040 | 5.1 |
| 15.7797 | 5.61625 | 5.5 |
| 16.7571 | 5.29080 | 4.8 |
| 18.0438 | 4.91632 | 2.7 |
| 19.2935 | 4.60059 | 13.8 |
| 19.6149 | 4.52218 | 11.2 |
| 20.7539 | 4.28004 | 8.4 |
| 21.7377 | 4.08852 | 10.5 |
| 22.7019 | 3.91701 | 11.9 |
| 23.9464 | 3.71617 | 5.6 |
| 25.8648 | 3.44474 | 5.7 |
| 29.0345 | 3.07549 | 4.5 |
| 30.0151 | 2.97721 | 0.8 |

Table 44 below provides the results of the XRPD performed on phosphoric acid salt Pattern 5. The XRPD exhibited sharp peaks, indicating the sample was composed of crystalline material. Significant peaks were observed in the XRPD on phosphoric acid salt Pattern 5 at about 3.9±0.2°, about 10.3±0.2°, about 10.6±0.2°, about 13.4±0.2°, about 14.1±0.2°, about 15.5±0.2°, about 19.5±0.2°, about 20.7±0.2°, about 23.2±0.2°, and about 23.6±0.2°.

TABLE 44
XRPD Peak List For Phosphoric Acid Salt Pattern 5

| Pos. [°2θ] | d-spacing [Å] | Rel. Int. [%] |
|---|---|---|
| 3.8903 | 22.71309 | 100.00 |
| 6.7501 | 13.09524 | 4.22 |
| 7.7645 | 11.38649 | 11.18 |
| 10.2747 | 8.60961 | 22.16 |
| 10.5978 | 8.34789 | 14.20 |
| 11.6668 | 7.58527 | 4.14 |
| 13.4388 | 6.58880 | 19.27 |
| 14.0769 | 6.29156 | 24.28 |
| 14.5729 | 6.07850 | 4.90 |
| 15.5326 | 5.70504 | 12.14 |
| 16.2258 | 5.46283 | 7.01 |
| 16.9373 | 5.23493 | 10.93 |
| 17.4629 | 5.07853 | 5.82 |
| 17.9384 | 4.94495 | 5.48 |
| 19.4979 | 4.55282 | 17.54 |
| 20.7233 | 4.28631 | 21.98 |
| 21.4358 | 4.14542 | 9.60 |
| 22.1410 | 4.01495 | 5.99 |
| 22.7266 | 3.91280 | 7.30 |
| 23.2378 | 3.82787 | 17.36 |
| 23.5640 | 3.77562 | 39.80 |
| 24.4259 | 3.64430 | 7.10 |
| 25.1357 | 3.54299 | 4.37 |
| 25.8936 | 3.44097 | 5.62 |
| 27.2288 | 3.27520 | 3.66 |
| 27.8924 | 3.19877 | 4.47 |
| 28.3334 | 3.14997 | 3.91 |
| 29.1522 | 3.06334 | 5.78 |
| 30.0086 | 2.97783 | 1.37 |
| 30.6596 | 2.91608 | 1.82 |
| 31.5297 | 2.83756 | 1.27 |
| 32.5447 | 2.75135 | 1.54 |

Table 45 below provides the results of the XRPD performed on phosphoric acid salt Pattern 6. The XRPD exhibited sharp peaks, indicating the sample was composed of crystalline material. Significant peaks were observed in the XRPD on phosphoric acid salt Pattern 6 at about 5.0±0.2°, about 6.9±0.2°, about 13.7±0.2°, about 17.9±0.2°, about 18.7±0.2°, about 19.6±0.2°, about 20.7±0.2°, about 21.1±0.2°, about 22.3±0.2°, and about 25.2±0.2°.

TABLE 45
XRPD Peak List For Phosphoric Acid Salt Pattern 6

| Pos. [°2θ] | d-spacing [Å] | Rel. Int. [%] |
|---|---|---|
| 5.0362 | 17.54727 | 66.57 |
| 5.8696 | 15.05756 | 19.54 |
| 6.8717 | 12.86375 | 63.45 |
| 8.5314 | 10.36460 | 9.80 |
| 8.8956 | 9.94101 | 4.89 |
| 9.7424 | 9.07878 | 8.29 |
| 10.5140 | 8.41417 | 5.52 |
| 11.7525 | 7.53016 | 20.92 |
| 11.9707 | 7.39334 | 12.35 |
| 12.6912 | 6.97521 | 11.75 |
| 13.1905 | 6.71228 | 11.86 |
| 13.7457 | 6.44237 | 100.00 |
| 14.1775 | 6.24714 | 11.85 |
| 15.1228 | 5.85869 | 27.05 |
| 15.2725 | 5.80161 | 21.13 |
| 16.2436 | 5.45688 | 13.61 |
| 17.0911 | 5.18815 | 34.83 |
| 17.3636 | 5.10734 | 28.33 |
| 17.9418 | 4.94405 | 48.22 |
| 18.6847 | 4.74910 | 44.15 |
| 18.9193 | 4.69075 | 16.65 |
| 19.5942 | 4.53067 | 66.11 |
| 20.6734 | 4.29654 | 41.06 |
| 21.1082 | 4.20900 | 69.47 |
| 21.4865 | 4.13575 | 18.63 |
| 21.8026 | 4.07649 | 17.38 |
| 22.3530 | 3.97734 | 55.50 |
| 22.9505 | 3.87513 | 18.90 |
| 23.2642 | 3.82042 | 10.09 |
| 24.0841 | 3.69525 | 21.97 |
| 24.3364 | 3.65750 | 19.71 |
| 25.2210 | 3.53119 | 62.48 |
| 25.6359 | 3.47497 | 29.42 |
| 27.0085 | 3.30141 | 12.57 |
| 27.6808 | 3.22274 | 17.84 |
| 28.3374 | 3.14954 | 11.74 |
| 28.6608 | 3.11474 | 9.73 |
| 29.1985 | 3.05858 | 5.14 |
| 30.8717 | 2.89652 | 9.52 |
| 31.5042 | 2.83980 | 6.50 |
| 32.8545 | 2.72611 | 2.71 |

Table 46 below provides the results of the XRPD performed on phosphoric acid salt Pattern 6* (* indicates slight shifts in XRPD peak positions in comparison with phosphoric acid Pattern 6.) The XRPD exhibited sharp peaks, indicating the sample was composed of crystalline material. Significant peaks were observed in the XRPD on phosphoric acid salt Pattern 6* at about 4.2±0.2°, about 4.7±0.2°, about 4.9±0.2°, about 5.1±0.2°, about 12.4±0.2°, about 13.6±0.2°, about 18.6±0.2°, about 19.6±0.2°, about 21.2±0.2°, about 22.4±0.2°, and about 25.0±0.2°.

TABLE 46
XRPD Peak List For Phosphoric Acid Salt Pattern 6*

| Pos. [°2θ] | d-spacing [Å] | Rel. Int. [%] |
|---|---|---|
| 4.2296 | 20.89142 | 59.67 |
| 4.6709 | 18.91863 | 100.00 |
| 4.8680 | 18.15307 | 80.25 |
| 5.0694 | 17.43245 | 80.45 |
| 5.8208 | 15.18375 | 9.43 |
| 6.7958 | 13.00722 | 14.89 |
| 7.5628 | 11.68971 | 4.89 |
| 8.9007 | 9.93538 | 9.63 |
| 9.7372 | 9.08363 | 15.88 |
| 11.7234 | 7.54875 | 20.77 |
| 11.9741 | 7.39129 | 24.33 |
| 12.4314 | 7.12041 | 58.45 |
| 13.2034 | 6.70576 | 28.30 |
| 13.6204 | 6.50137 | 77.53 |
| 15.2466 | 5.81140 | 43.68 |
| 16.1842 | 5.47677 | 28.52 |
| 17.0052 | 5.21417 | 32.42 |
| 17.4750 | 5.07502 | 30.98 |
| 17.9698 | 4.93640 | 44.57 |
| 18.6465 | 4.75875 | 62.40 |
| 19.5815 | 4.53357 | 60.54 |
| 20.1623 | 4.40428 | 57.33 |
| 20.5462 | 4.32285 | 46.36 |
| 21.2429 | 4.18263 | 71.94 |
| 22.3694 | 3.97447 | 58.92 |
| 23.6914 | 3.75559 | 27.72 |
| 24.3318 | 3.65819 | 39.65 |
| 24.9801 | 3.56470 | 63.29 |
| 25.6347 | 3.47226 | 23.25 |
| 27.5038 | 3.24307 | 17.66 |
| 30.3498 | 2.94514 | 7.12 |

Table 47 below provides the results of the XRPD performed on phosphoric acid salt Pattern 7. The XRPD exhibited sharp peaks, indicating the sample was composed of crystalline material. Significant peaks were observed in the XRPD on phosphoric acid salt Pattern 7 at about 3.9±0.2°, about 10.2±0.2°, about 13.4±0.2°, about 14.3±0.2°, about 16.9±0.2°, about 19.6±0.2°, about 5 20.4±0.2°, about 21.1±0.2°, about 22.5±0.2°, and about 23.3±0.2°.

TABLE 47
XRPD Peak List For Phosphoric Acid Salt Pattern 7

| Pos. [°2θ] | d-spacing [Å] | Rel. Int. [%] |
|---|---|---|
| 3.9117 | 22.58844 | 100 |
| 5.7243 | 15.43940 | 1.67 |
| 7.2709 | 12.15830 | 5.30 |
| 7.7610 | 11.39168 | 8.55 |
| 7.9274 | 11.15284 | 6.20 |
| 10.1983 | 8.67393 | 32.35 |
| 10.7312 | 8.24442 | 4.67 |
| 11.6996 | 7.56405 | 3.98 |
| 12.7589 | 6.93836 | 6.51 |
| 13.3788 | 6.61823 | 18.95 |
| 13.7435 | 6.44341 | 14.00 |
| 14.2655 | 6.20879 | 22.61 |
| 15.2237 | 5.82009 | 8.93 |
| 15.6609 | 5.65858 | 13.76 |
| 16.2203 | 5.46468 | 8.87 |
| 16.9209 | 5.23995 | 21.37 |
| 17.4620 | 5.07878 | 10.28 |
| 17.8527 | 4.96849 | 5.43 |
| 18.2408 | 4.86366 | 7.22 |
| 19.6462 | 4.51878 | 18.57 |
| 20.3961 | 4.35432 | 20.84 |
| 21.1164 | 4.20739 | 19.97 |
| 21.5041 | 4.13239 | 7.38 |
| 21.8843 | 4.06145 | 8.14 |
| 22.5118 | 3.94964 | 14.66 |
| 23.2838 | 3.82041 | 79.59 |
| 25.1062 | 3.54708 | 12.69 |
| 25.9229 | 3.43714 | 5.87 |
| 26.9689 | 3.30617 | 4.24 |
| 27.7148 | 3.21886 | 8.74 |
| 28.3354 | 3.14976 | 4.83 |
| 29.9330 | 2.98519 | 1.90 |
| 33.2800 | 2.69222 | 1.40 |
| 34.0441 | 2.63353 | 1.98 |

Table 48 below provides the results of the XRPD performed on phosphoric acid salt Pattern 7* (* indicates slight shifts in XRPD peak positions in comparison with phosphoric acid Pattern 7.) The XRPD exhibited sharp peaks, indicating the sample was composed of crystalline material. Significant peaks were observed in the XRPD on phosphoric acid salt Pattern 7* at about 3.9±0.2°, about 10.2±0.2°, about 13.4±0.2°, about 14.2±0.2°, about 15.6±0.2°, about 16.9±0.2°, about 19.6±0.2°, about 20.9±0.2°, about 23.3±0.2°, and about 23.6±0.2°.

TABLE 48
XRPD Peak List For Phosphoric Acid Salt Pattern 7*

| Pos. [°2θ] | d-spacing [Å] | Rel. Int. [%] |
|---|---|---|
| 3.9031 | 22.63812 | 100.00 |
| 6.7722 | 13.05258 | 4.01 |
| 7.2642 | 12.16952 | 4.20 |
| 7.7941 | 11.34332 | 9.27 |
| 10.2069 | 8.66666 | 26.65 |
| 10.7522 | 8.22835 | 3.95 |
| 11.7031 | 7.56179 | 4.09 |
| 12.7803 | 6.92675 | 4.08 |
| 13.3913 | 6.61205 | 13.05 |
| 14.1876 | 6.24269 | 26.84 |
| 14.5971 | 6.06848 | 6.07 |
| 15.2237 | 5.82009 | 6.03 |
| 15.6185 | 5.67384 | 16.13 |
| 16.2264 | 5.46263 | 9.82 |
| 16.6401 | 5.32773 | 8.33 |
| 16.8954 | 5.24780 | 16.28 |
| 17.5186 | 5.06249 | 6.63 |
| 18.3221 | 4.84225 | 5.90 |
| 19.2624 | 4.60795 | 7.65 |
| 19.5723 | 4.53570 | 18.41 |
| 20.4244 | 4.34835 | 12.56 |
| 20.8716 | 4.25617 | 18.06 |
| 21.4301 | 4.14651 | 9.98 |
| 22.5329 | 3.94600 | 11.91 |
| 23.2878 | 3.81977 | 42.79 |
| 23.6329 | 3.76476 | 31.93 |
| 24.4494 | 3.63784 | 5.07 |
| 25.0929 | 3.54893 | 11.15 |
| 25.5371 | 3.48819 | 8.44 |
| 26.0426 | 3.42162 | 6.50 |
| 26.5858 | 3.35293 | 4.65 |
| 27.0382 | 3.29512 | 3.03 |
| 27.7246 | 3.21774 | 7.19 |
| 28.4076 | 3.14192 | 6.19 |
| 29.9628 | 2.98229 | 4.12 |
| 31.4601 | 2.84368 | 3.71 |
| 32.5324 | 2.75236 | 3.01 |
| 34.1078 | 2.62875 | 3.73 |

Table 49 below provides the results of the XRPD performed on phosphoric acid salt Pattern 8. The XRPD exhibited sharp peaks, indicating the sample was composed of crystalline material. Significant peaks were observed in the XRPD on phosphoric acid salt Pattern 8 at about 3.8±0.2°, about 10.0±0.2°, about 10.6±0.2°, about 14.1±0.2°, about 16.6±0.2°, about 19.3±0.2°, about 20.8±0.2°, about 22.3±0.2°, about 22.4±0.2°, and about 23.9±0.2°.

TABLE 49
XRPD Peak List For Phosphoric Acid Salt Pattern 8

| Pos. [°2θ] | d-spacing [Å] | Rel. Int. [%] |
|---|---|---|
| 3.8443 | 22.98475 | 100.00 |
| 6.6759 | 13.24065 | 3.17 |
| 7.5976 | 11.63628 | 7.29 |
| 10.0106 | 8.83617 | 19.89 |
| 10.5876 | 8.35588 | 25.98 |
| 11.4349 | 7.73854 | 2.11 |
| 13.1354 | 6.74032 | 9.22 |
| 13.5805 | 6.52036 | 11.83 |
| 14.0758 | 6.29203 | 35.59 |
| 15.6964 | 5.64587 | 16.18 |
| 16.5819 | 5.34631 | 16.45 |
| 17.0672 | 5.19536 | 6.46 |
| 17.5392 | 5.05661 | 7.36 |
| 18.0388 | 4.91767 | 5.49 |
| 18.7097 | 4.74281 | 4.57 |
| 19.0238 | 4.66520 | 9.77 |
| 19.3051 | 4.59786 | 18.71 |
| 19.6637 | 4.51482 | 8.51 |
| 20.2233 | 4.39113 | 6.10 |
| 20.8510 | 4.26033 | 21.22 |
| 21.1335 | 4.20403 | 10.85 |
| 21.4620 | 4.14040 | 12.31 |
| 21.7134 | 4.09304 | 8.55 |
| 22.3002 | 3.98334 | 17.11 |
| 22.3650 | 3.97523 | 19.75 |
| 22.7192 | 3.91405 | 14.08 |
| 22.8862 | 3.88587 | 14.93 |
| 23.2436 | 3.82693 | 8.03 |
| 23.8808 | 3.72623 | 48.09 |
| 24.4233 | 3.64469 | 8.22 |
| 25.0084 | 3.56072 | 6.97 |
| 25.8820 | 3.44249 | 7.23 |
| 26.9849 | 3.30424 | 3.95 |
| 27.7995 | 3.20924 | 3.19 |
| 28.3687 | 3.14613 | 6.97 |
| 29.1348 | 3.06513 | 9.69 |
| 30.0148 | 2.97724 | 1.45 |
| 30.5682 | 2.92459 | 1.66 |
| 31.7847 | 2.81537 | 2.05 |
| 32.7471 | 2.73480 | 1.60 |
| 33.3757 | 2.68472 | 0.65 |

Table 50 below provides the results of the XRPD performed on phosphoric acid salt Pattern 8*. (* indicates slight shifts in XRPD peak positions in comparison with phosphoric acid Pattern 8.) The XRPD exhibited sharp peaks, indicating the sample was composed of crystalline material. Significant peaks were observed in the XRPD on phosphoric acid salt Pattern 8* at about 3.9±0.2°, about 10.2±0.2°, about 10.6±0.2°, about 13.4±0.2°, about 14.1±0.2°, about 15.6±0.2°, about 19.3±0.2°, about 20.8±0.2°, about 23.2±0.2°, and about 23.7±0.2°.

TABLE 50
XRPD Peak List For Phosphoric Acid Salt Pattern 8*

| Pos. [°2θ] | d-spacing [Å] | Rel. Int. [%] |
|---|---|---|
| 3.8720 | 22.82005 | 100.00 |
| 6.7497 | 13.09604 | 3.99 |
| 7.7131 | 11.46231 | 9.28 |
| 10.1808 | 8.68878 | 17.13 |
| 10.5940 | 8.35084 | 25.91 |
| 11.5916 | 7.63428 | 3.15 |
| 13.4032 | 6.60624 | 13.64 |
| 13.5912 | 6.51527 | 11.45 |
| 14.1509 | 6.25882 | 27.01 |
| 15.6193 | 5.67356 | 14.57 |
| 16.6414 | 5.32732 | 10.57 |
| 17.0744 | 5.19320 | 6.45 |
| 17.5508 | 5.05329 | 6.63 |
| 18.0545 | 4.91343 | 5.69 |
| 19.0349 | 4.66250 | 6.04 |
| 19.3334 | 4.59119 | 15.23 |
| 20.7843 | 4.27385 | 22.77 |
| 21.4455 | 4.14356 | 11.50 |
| 21.7218 | 4.09147 | 7.20 |
| 22.2912 | 3.98824 | 11.02 |
| 22.7201 | 3.91391 | 10.17 |
| 23.2451 | 3.82669 | 16.37 |
| 23.6627 | 3.76009 | 40.57 |
| 24.4122 | 3.64632 | 8.08 |
| 25.8728 | 3.44369 | 5.68 |
| 28.0749 | 3.17839 | 4.37 |
| 28.4135 | 3.14127 | 4.12 |
| 29.1579 | 3.06275 | 8.17 |
| 30.0361 | 2.97517 | 1.34 |
| 30.6652 | 2.91556 | 1.19 |
| 31.5962 | 2.83174 | 1.18 |
| 32.6775 | 2.74047 | 1.84 |

Table 51 below provides the results of the XRPD performed on (+)-L-tartaric acid salt Pattern 1. The XRPD exhibited sharp peaks, indicating the sample was composed of crystalline material. Significant peaks were observed in the XRPD on (+)-L-tartaric acid salt Pattern 1 at about 7.7±0.2°, about 14.2±0.2°, about 15.5±0.2°, about 15.9±0.2°, about 17.3±0.2°, about 19.1±0.2°, about 19.7±0.2°, about 20.8±0.2°, about 23.3±0.2°, and about 24.5±0.2°.

TABLE 51
XRPD Peak List For (+)-L-Tartaric Acid Salt Pattern 1

| Pos. [°2θ] | d-spacing [Å] | Rel. Int. [%] |
|---|---|---|
| 7.6995 | 11.48244 | 22.51 |
| 8.3104 | 10.63967 | 8.55 |
| 10.0544 | 8.79778 | 7.71 |
| 10.4780 | 8.44300 | 9.52 |
| 10.7413 | 8.23663 | 7.70 |
| 11.6053 | 7.62530 | 2.40 |
| 12.4910 | 7.08654 | 0.88 |
| 14.2233 | 6.22709 | 32.23 |
| 15.3529 | 5.77141 | 12.81 |
| 15.5089 | 5.71369 | 18.47 |
| 15.9222 | 5.56629 | 19.75 |
| 17.2798 | 5.13193 | 99.14 |
| 19.0625 | 4.65582 | 45.42 |
| 19.7549 | 4.49418 | 22.12 |
| 20.8368 | 4.26322 | 100.00 |
| 21.2577 | 4.17974 | 9.95 |
| 21.6123 | 4.11195 | 13.34 |
| 22.0573 | 4.03000 | 4.90 |
| 22.2831 | 3.98967 | 14.20 |
| 22.9751 | 3.87104 | 6.50 |
| 23.3450 | 3.81054 | 29.16 |
| 24.4884 | 3.63514 | 48.40 |
| 25.1675 | 3.53857 | 10.23 |
| 26.0939 | 3.41502 | 7.91 |
| 26.5369 | 3.35900 | 6.21 |
| 28.5736 | 3.12404 | 8.35 |
| 29.4256 | 3.03550 | 6.60 |
| 30.1623 | 2.96301 | 10.71 |
| 30.9038 | 2.89359 | 9.93 |
| 31.2621 | 2.86124 | 5.30 |
| 32.2167 | 2.77861 | 2.11 |
| 32.6354 | 2.74391 | 1.55 |
| 33.5125 | 2.67407 | 0.87 |
| 34.0497 | 2.63311 | 3.68 |

Table 52 below provides the results of the XRPD performed on (+)-L-tartaric acid salt Pattern 1* (* indicates slight shifts in XRPD peak positions in comparison with (+)-L-tartaric acid salt Pattern 1.) The XRPD exhibited sharp peaks, indicating the sample was composed of crystalline material. Significant peaks were observed in the XRPD on (+)-L-tartaric acid salt Pattern 1* at about 7.7±0.2°, about 14.2±0.2°, about 15.9±0.2°, about 17.3±0.2°, about 19.0±0.2°, about 20.8±0.2°, about 22.3±0.2°, about 23.3±0.2°, about 24.4±0.2°, and about 25.1±0.2°.

TABLE 52
XRPD Peak List For (+)-L-Tartaric Acid Salt Pattern 1*

| Pos. [°2θ] | d-spacing [Å] | Rel. Int. [%] |
|---|---|---|
| 4.0281 | 21.93615 | 27.07 |
| 4.8592 | 18.18616 | 12.49 |
| 7.7062 | 11.47246 | 29.04 |
| 8.3091 | 10.64137 | 24.39 |
| 8.4566 | 10.45611 | 20.40 |
| 10.0415 | 8.80900 | 17.40 |
| 10.5808 | 8.36119 | 9.05 |
| 14.2146 | 6.23089 | 60.91 |
| 14.6593 | 6.04289 | 22.96 |
| 15.4374 | 5.74000 | 27.43 |
| 15.8779 | 5.58172 | 45.03 |
| 17.2658 | 5.13607 | 95.35 |
| 19.0498 | 4.65891 | 83.75 |
| 19.7345 | 4.49877 | 20.58 |
| 20.8117 | 4.26828 | 100.00 |
| 21.2033 | 4.19034 | 20.65 |
| 21.5794 | 4.11815 | 21.17 |
| 22.2642 | 3.99301 | 40.28 |
| 23.3215 | 3.81432 | 50.20 |
| 24.4534 | 3.64027 | 52.94 |
| 25.0877 | 3.54966 | 32.58 |
| 26.0588 | 3.41954 | 12.90 |
| 26.5012 | 3.36345 | 10.70 |
| 27.7516 | 3.21468 | 5.09 |
| 28.5399 | 3.12765 | 17.50 |
| 29.3873 | 3.03936 | 6.21 |
| 30.1075 | 2.96828 | 12.26 |
| 30.8403 | 2.89940 | 7.61 |

Table 53 below provides the results of the XRPD performed on (+)-L-tartaric acid salt Pattern 2. The XRPD exhibited sharp peaks, indicating the sample was composed of crystalline material. Significant peaks were observed in the XRPD on (+)-L-tartaric acid salt Pattern 2 at about 4.9±0.2°, about 9.7±0.2°, about 11.9±0.2°, about 15.2±0.2°, about 19.3±0.2°, about 19.6±0.2°, 5 about 20.8±0.2°, about 21.5±0.2°, about 21.7±0.2°, and about 24.9±0.2°.

TABLE 53
XRPD Peak List For (+)-L-Tartaric Acid Salt Pattern 2

| Pos. [°2θ] | d-spacing [Å] | Rel. Int. [%] |
|---|---|---|
| 4.8586 | 18.18812 | 84.11 |
| 7.6690 | 11.51852 | 6.95 |
| 7.9343 | 11.14317 | 12.67 |
| 8.2632 | 10.70037 | 7.14 |
| 8.9035 | 9.93226 | 38.94 |
| 9.7423 | 9.07890 | 49.32 |
| 10.5699 | 8.36985 | 19.04 |
| 11.9549 | 7.40313 | 53.43 |
| 13.1971 | 6.70896 | 41.25 |
| 14.2299 | 6.22426 | 24.74 |
| 15.2468 | 5.81133 | 94.65 |
| 15.8789 | 5.58138 | 26.59 |
| 16.0891 | 5.50892 | 32.86 |
| 16.9001 | 5.24635 | 19.19 |
| 17.2607 | 5.13757 | 33.75 |
| 17.9428 | 4.94377 | 34.21 |
| 19.0021 | 4.67050 | 44.79 |
| 19.3193 | 4.59451 | 55.26 |
| 19.5861 | 4.53253 | 51.05 |
| 20.5821 | 4.31538 | 41.28 |
| 20.8389 | 4.26278 | 47.26 |
| 21.4911 | 4.13487 | 54.24 |
| 21.7131 | 4.09310 | 100.00 |
| 22.3037 | 3.98603 | 26.54 |
| 23.3217 | 3.81428 | 17.36 |
| 23.9938 | 3.70895 | 24.97 |
| 24.5086 | 3.63220 | 36.60 |
| 24.9328 | 3.57135 | 49.33 |
| 25.8359 | 3.44853 | 13.35 |
| 26.5993 | 3.35126 | 10.54 |
| 27.2493 | 3.27278 | 13.92 |
| 28.2768 | 3.15615 | 19.24 |
| 28.6239 | 3.11866 | 19.62 |
| 29.5240 | 3.02560 | 5.94 |
| 30.1487 | 2.96432 | 10.99 |
| 30.7841 | 2.90457 | 9.66 |
| 31.3909 | 2.84979 | 11.46 |
| 32.0124 | 2.79587 | 6.70 |
| 32.6525 | 2.74024 | 6.16 |
| 33.0580 | 2.70755 | 5.78 |

Table 54 below provides the results of the XRPD performed on citric acid salt Pattern 1. The XRPD exhibited sharp peaks, indicating the sample was composed of crystalline material. Significant peaks were observed in the XRPD on citric acid salt Pattern 1 at about 4.2±0.2°, about 7.8±0.2°, about 13.7±0.2°, about 14.9±0.2°, about 15.8±0.2°, about 17.5±0.2°, about 18.5±0.2°, about 19.0±0.2°, about 21.4±0.2°, and about 21.8±0.2°.

TABLE 54
XRPD Peak List For Citric Acid Salt Pattern 1

| Pos. [°2θ] | d-spacing [Å] | Rel. Int. [%] |
|---|---|---|
| 4.2493 | 20.79499 | 100.00 |
| 4.8681 | 18.15282 | 22.07 |
| 6.8491 | 12.90614 | 8.34 |
| 7.7867 | 11.35411 | 32.26 |
| 8.3385 | 10.60396 | 7.52 |
| 8.5058 | 10.39570 | 5.35 |
| 8.8997 | 9.93643 | 5.91 |
| 9.7331 | 9.08746 | 8.38 |
| 10.5172 | 8.41163 | 11.23 |
| 11.3488 | 7.79704 | 6.93 |
| 11.9536 | 7.40388 | 11.58 |
| 12.7804 | 6.92671 | 10.02 |
| 13.2225 | 6.69612 | 6.11 |
| 13.7483 | 6.44119 | 27.15 |
| 14.9548 | 5.92412 | 40.55 |
| 15.2621 | 5.80551 | 21.08 |
| 15.6473 | 5.66348 | 16.60 |
| 15.8519 | 5.59084 | 27.36 |
| 16.0940 | 5.50727 | 10.99 |
| 16.7492 | 5.29327 | 19.51 |
| 17.0802 | 5.19143 | 12.62 |
| 17.4988 | 5.06817 | 24.21 |
| 17.9715 | 4.93594 | 10.43 |
| 18.5495 | 4.78342 | 37.87 |
| 18.9137 | 4.69212 | 21.60 |
| 19.0527 | 4.65820 | 26.60 |
| 19.3216 | 4.59398 | 13.59 |
| 19.5948 | 4.53052 | 15.69 |
| 19.9248 | 4.45623 | 8.37 |
| 20.5838 | 4.31503 | 22.48 |
| 21.4283 | 4.14684 | 49.91 |
| 21.7584 | 4.08467 | 27.89 |
| 22.3260 | 3.98209 | 14.03 |
| 22.7325 | 3.91181 | 16.49 |
| 23.4451 | 3.79450 | 9.77 |
| 24.3882 | 3.64986 | 12.74 |
| 24.9287 | 3.57194 | 11.78 |
| 25.8730 | 3.44366 | 11.31 |
| 26.6846 | 3.34075 | 5.22 |
| 27.5564 | 3.23700 | 16.96 |
| 27.7352 | 3.21653 | 22.83 |
| 28.2953 | 3.15412 | 6.70 |
| 28.6857 | 3.11209 | 5.40 |
| 29.0282 | 3.07615 | 3.97 |
| 29.7251 | 3.00559 | 4.83 |
| 30.1559 | 2.96362 | 4.81 |
| 30.7890 | 2.90412 | 5.43 |
| 31.5219 | 2.83825 | 6.84 |
| 31.9849 | 2.79821 | 8.81 |
| 32.8606 | 2.72562 | 4.31 |
| 34.0007 | 2.63678 | 4.81 |

Table 55 below provides the results of the XRPD performed on citric acid salt Pattern 2. The XRPD exhibited sharp peaks, indicating the sample was composed of crystalline material. Significant peaks were observed in the XRPD on citric acid salt Pattern 2 at about 12.8±0.2°, about 13.8±0.2°, about 15.0±0.2°, about 17.6±0.2°, about 18.2±0.2°, about 19.3±0.2°, about 19.7±0.2°, about 21.5±0.2°, about 22.3±0.2°, and about 28.3±0.2°.

TABLE 55
XRPD Peak List For Citric Acid Salt Pattern 2

| Pos. [°2θ] | d-spacing [Å] | Rel. Int. [%] |
|---|---|---|
| 5.2545 | 16.81884 | 11.85 |
| 9.6035 | 9.20979 | 19.97 |
| 9.8326 | 8.99575 | 16.55 |
| 10.6231 | 8.32806 | 26.11 |
| 11.2477 | 7.86695 | 10.55 |
| 12.8085 | 6.91157 | 100.00 |
| 13.3841 | 6.61559 | 27.04 |
| 13.7934 | 6.42020 | 34.49 |
| 14.1542 | 6.25738 | 15.08 |
| 14.7584 | 6.00254 | 23.40 |
| 14.9980 | 5.90717 | 31.85 |
| 15.4803 | 5.72419 | 12.87 |
| 15.8256 | 5.60005 | 10.28 |
| 16.9976 | 5.21647 | 7.00 |
| 17.6377 | 5.02860 | 28.80 |
| 18.2022 | 4.87388 | 43.20 |
| 19.3139 | 4.59579 | 79.71 |
| 19.6920 | 4.50839 | 31.54 |
| 21.4762 | 4.13771 | 59.42 |
| 22.2746 | 3.99117 | 40.74 |
| 24.3852 | 3.65029 | 15.54 |
| 25.9007 | 3.44004 | 9.68 |
| 26.3291 | 3.38504 | 7.46 |
| 28.2909 | 3.15461 | 47.94 |
| 29.1161 | 3.06705 | 19.22 |
| 29.8584 | 2.99248 | 13.12 |
| 32.3402 | 2.76828 | 1.54 |

Table 56 below provides the results of the XRPD performed on citric acid salt Pattern 4. The XRPD exhibited sharp peaks, indicating the sample was composed of crystalline material. Significant peaks were observed in the XRPD on citric acid salt Pattern 4 at about 10.0±0.2°, about 10.6±0.2°, about 13.3±0.2°, about 14.3±0.2°, about 14.8±0.2°, about 18.4±0.2°, about 19.4±0.2°, about 20.4±0.2°, about 23.0±0.2°, and about 28.4±0.2°.

TABLE 56
XRPD Peak List For Citric Acid Salt Pattern 4

| Pos. [°2θ] | d-spacing [Å] | Rel. Int. [%] |
|---|---|---|
| 9.9767 | 8.86614 | 20.38 |
| 10.5950 | 8.35007 | 27.39 |
| 12.5416 | 7.05805 | 12.96 |
| 13.3381 | 6.63831 | 100.00 |
| 14.3427 | 6.17554 | 20.50 |
| 14.8375 | 5.97070 | 25.39 |
| 16.2044 | 5.46998 | 13.31 |
| 17.2923 | 5.12823 | 18.10 |
| 18.3828 | 4.82640 | 47.99 |
| 19.4208 | 4.57074 | 80.80 |
| 20.0493 | 4.42884 | 17.57 |
| 20.4553 | 4.34185 | 21.33 |
| 21.1690 | 4.19705 | 12.27 |
| 21.8727 | 4.06359 | 16.01 |
| 23.0156 | 3.86432 | 23.71 |
| 23.8552 | 3.73018 | 10.09 |
| 24.9432 | 3.56989 | 15.68 |
| 25.3969 | 3.50713 | 17.03 |
| 28.3585 | 3.14724 | 37.59 |
| 28.9550 | 3.08375 | 16.13 |
| 32.2641 | 2.77463 | 10.81 |
| 33.9846 | 2.63800 | 6.21 |

Table 57 below provides the results of the XRPD performed on hydrobromic acid salt Pattern 1. The XRPD exhibited sharp peaks, indicating the sample was composed of crystalline material. Significant peaks were observed in the XRPD on hydrobromic acid salt Pattern 1 at about 10.6±0.2°, about 13.4±0.2°, about 17.0±0.2°, about 19.0±0.2°, about 19.5±0.2°, about 20.6±0.2°, about 21.8±0.2°, about 22.3±0.2°, about 22.6±0.2°, about 25.8±0.2°, and about 29.1±0.2°.

TABLE 57
XRPD Peak List For Hydrobromic Acid Salt Pattern 1

| Pos. [°2θ] | d-spacing [Å] | Rel. Int. [%] |
|---|---|---|
| 10.6443 | 8.31147 | 100.00 |
| 13.0537 | 6.78228 | 8.34 |
| 13.3807 | 6.61730 | 25.99 |
| 15.7351 | 5.63207 | 17.85 |
| 16.3329 | 5.42724 | 9.63 |
| 17.0215 | 5.20922 | 20.93 |
| 18.7350 | 4.73647 | 8.78 |
| 19.0087 | 4.66889 | 25.68 |
| 19.2797 | 4.60386 | 14.94 |
| 19.4813 | 4.55666 | 34.04 |
| 20.5846 | 4.31486 | 24.58 |
| 21.1533 | 4.20014 | 6.34 |
| 21.4000 | 4.15227 | 9.24 |
| 21.7799 | 4.08068 | 30.20 |
| 22.3232 | 3.98258 | 26.53 |
| 22.6450 | 3.92671 | 28.69 |
| 23.5526 | 3.77742 | 2.24 |
| 24.3601 | 3.65400 | 4.09 |
| 25.8383 | 3.44821 | 24.44 |
| 26.3181 | 3.38643 | 5.55 |
| 26.6899 | 3.34009 | 6.03 |
| 27.0544 | 3.29591 | 5.79 |
| 27.3308 | 3.26321 | 7.11 |
| 27.6620 | 3.22488 | 4.04 |
| 27.8780 | 3.20038 | 2.46 |
| 28.6163 | 3.11948 | 6.48 |
| 29.0813 | 3.07064 | 46.37 |
| 29.9489 | 2.98364 | 10.31 |
| 30.5194 | 2.92673 | 2.58 |
| 30.7322 | 2.90935 | 3.15 |
| 31.7173 | 2.82121 | 6.49 |
| 32.5454 | 2.75129 | 3.90 |
| 33.0208 | 2.71276 | 5.53 |
| 33.5147 | 2.67390 | 6.30 |
| 33.8487 | 2.64828 | 6.84 |
| 34.5798 | 2.59395 | 5.70 |

Table 58 below provides the results of the XRPD performed on hydrobromic acid salt Pattern 2. The XRPD exhibited sharp peaks, indicating the sample was composed of crystalline material. Significant peaks were observed in the XRPD on hydrobromic acid salt Pattern 2 at about 4.9±0.2°, about 8.8±0.2°, about 10.6±0.2°, about 12.0±0.2°, about 14.6±0.2°, about 20.2±0.2°, about 20.9±0.2°, about 21.3±0.2°, about 21.6±0.2°, about 24.5±0.2°, and about 24.8±0.2°.

TABLE 58
XRPD Peak List For Hydrobromic Acid Salt Pattern 2

| Pos. [°2θ] | d-spacing [Å] | Rel. Int. [%] |
|---|---|---|
| 4.8568 | 18.19506 | 48.23 |
| 6.0054 | 14.71742 | 17.77 |
| 8.7660 | 10.08771 | 100.00 |
| 9.2597 | 9.54304 | 7.24 |
| 9.5936 | 9.21931 | 9.64 |
| 10.5625 | 8.37564 | 40.92 |
| 11.2390 | 7.87299 | 8.04 |
| 11.9850 | 7.38460 | 29.66 |
| 12.4620 | 7.10295 | 11.98 |
| 13.1012 | 6.75780 | 10.99 |
| 14.5702 | 6.07963 | 28.08 |
| 15.1861 | 5.83440 | 9.21 |
| 15.6226 | 5.67235 | 15.67 |
| 16.0626 | 5.51796 | 17.26 |
| 16.8173 | 5.27200 | 9.29 |
| 17.5569 | 5.05154 | 12.97 |
| 19.0175 | 4.66673 | 11.52 |
| 19.5811 | 4.53367 | 21.61 |
| 20.1637 | 4.40398 | 71.02 |
| 20.7066 | 4.28617 | 23.43 |
| 20.8786 | 4.25477 | 26.22 |
| 21.3315 | 4.16544 | 36.74 |
| 21.6389 | 4.10697 | 26.88 |
| 22.4348 | 3.96303 | 24.54 |
| 23.7120 | 3.75238 | 16.75 |
| 24.1999 | 3.67478 | 17.90 |
| 24.5378 | 3.62794 | 28.15 |
| 24.7706 | 3.59437 | 26.40 |
| 25.8049 | 3.45260 | 17.70 |
| 26.9035 | 3.31406 | 13.20 |
| 28.0363 | 3.18004 | 6.06 |
| 28.6381 | 3.11715 | 15.67 |
| 29.3910 | 3.03899 | 13.25 |
| 30.7181 | 2.91065 | 4.47 |
| 31.6414 | 2.82780 | 10.52 |
| 32.6020 | 2.74664 | 9.28 |
| 33.8639 | 2.64712 | 10.68 |

Table 59 below provides the results of the XRPD performed on benzenesulfonic acid salt Pattern 1. The XRPD exhibited sharp peaks, indicating the sample was composed of crystalline material. Significant peaks were observed in the XRPD on benzenesulfonic acid salt Pattern 1 at about 4.8±0.2°, about 13.4±0.2°, about 14.7±0.2°, about 14.9±0.2°, about 15.7±0.2°, about 18.7±0.2°, about 20.0±0.2°, about 23.5±0.2°, about 25.1±0.2°, about 27.7±0.2°, and about 28.1±0.2°.

TABLE 59
XRPD Peak List For Benzenesulfonic Acid Salt Pattern 1

| Pos. [°2θ] | d-spacing [Å] | Rel. Int. [%] |
|---|---|---|
| 4.7782 | 18.49423 | 100.00 |
| 7.2700 | 12.15978 | 19.57 |
| 7.7766 | 11.36887 | 16.67 |
| 8.2299 | 10.74362 | 19.30 |
| 10.7466 | 8.23262 | 14.86 |
| 11.5082 | 7.68943 | 6.39 |
| 12.5202 | 7.07006 | 10.38 |
| 13.0261 | 6.79662 | 23.73 |
| 13.3660 | 6.62453 | 35.90 |
| 13.6747 | 6.47566 | 22.87 |
| 13.9775 | 6.33606 | 22.21 |
| 14.6579 | 6.04343 | 55.42 |
| 14.9104 | 5.94168 | 82.25 |
| 15.6698 | 5.65540 | 49.45 |
| 16.4898 | 5.37595 | 21.54 |
| 16.6680 | 5.31887 | 22.41 |
| 16.9691 | 5.22517 | 28.92 |
| 17.4349 | 5.08660 | 17.29 |
| 18.6698 | 4.75285 | 35.53 |
| 19.2709 | 4.60595 | 33.60 |
| 19.9752 | 4.44510 | 39.11 |
| 20.5695 | 4.31800 | 15.67 |
| 21.1242 | 4.20585 | 16.32 |
| 21.3616 | 4.15620 | 12.59 |
| 21.7208 | 4.09165 | 23.73 |
| 21.9954 | 4.04119 | 22.42 |
| 22.4221 | 3.96197 | 8.02 |
| 23.0389 | 3.86047 | 19.54 |
| 23.4580 | 3.79243 | 42.18 |
| 24.0935 | 3.69383 | 23.57 |
| 25.0814 | 3.55053 | 42.80 |
| 25.6379 | 3.47471 | 19.93 |
| 26.0184 | 3.42475 | 27.65 |
| 26.2757 | 3.39179 | 33.35 |
| 26.6716 | 3.34234 | 28.36 |
| 27.7409 | 3.21589 | 42.51 |
| 28.0930 | 3.17638 | 34.87 |
| 29.5442 | 3.02358 | 12.25 |
| 30.4316 | 2.93740 | 7.87 |
| 31.1198 | 2.87400 | 11.48 |
| 31.9354 | 2.80243 | 5.65 |
| 32.5862 | 2.74794 | 3.92 |
| 33.9912 | 2.63750 | 7.49 |

Table 60 below provides the results of the XRPD performed on benzenesulfonic acid salt Pattern 2. The XRPD exhibited sharp peaks, indicating the sample was composed of crystalline material. Significant peaks were observed in the XRPD on benzenesulfonic acid salt Pattern 2 at about 6.1±0.2°, about 12.3±0.2°, about 13.2±0.2°, about 18.5±0.2°, about 18.8±0.2°, about 19.0±0.2°, about 19.4±0.2°, about 19.8±0.2°, about 20.5±0.2°, and about 25.5±0.2°.

TABLE 60
XRPD Peak List For Benzenesulfonic Acid Salt Pattern 2

| Pos. [°2θ] | d-spacing [Å] | Rel. Int. [%] |
|---|---|---|
| 3.122 | 28.3003 | 11.88 |
| 6.116 | 14.4515 | 13.91 |
| 6.611 | 13.3712 | 2.91 |
| 7.405 | 11.9385 | 4.36 |
| 7.966 | 11.0987 | 11.49 |
| 8.660 | 10.2107 | 11.19 |
| 9.144 | 9.6714 | 7.62 |
| 12.300 | 7.1962 | 63.54 |
| 13.210 | 6.7026 | 27.38 |
| 13.962 | 6.3430 | 4.53 |
| 15.280 | 5.7942 | 3.51 |
| 15.706 | 5.6425 | 5.61 |
| 16.484 | 5.3778 | 6.95 |
| 17.179 | 5.1618 | 5.81 |
| 18.456 | 4.8075 | 44.48 |
| 18.788 | 4.7233 | 25.25 |
| 19.037 | 4.6621 | 33.07 |
| 19.396 | 4.5766 | 100.00 |
| 19.782 | 4.4882 | 26.42 |
| 20.455 | 4.3419 | 11.94 |
| 21.045 | 4.2216 | 8.60 |
| 21.885 | 4.0613 | 7.76 |
| 22.550 | 3.9399 | 5.55 |
| 23.380 | 3.8049 | 6.55 |
| 23.925 | 3.7195 | 4.99 |
| 25.546 | 3.4870 | 15.78 |
| 26.023 | 3.4242 | 9.93 |
| 28.911 | 3.0884 | 5.90 |
| 31.791 | 2.8148 | 1.99 |
| 33.920 | 2.6429 | 2.92 |

Table 61 below provides the results of the XRPD performed on benzenesulfonic acid salt Pattern 3. The XRPD exhibited sharp peaks, indicating the sample was composed of crystalline material. Significant peaks were observed in the XRPD on benzenesulfonic acid salt Pattern 3 at about 5.5±0.2°, about 9.8±0.2°, about 11.9±0.2°, about 12.2±0.2°, about 17.3±0.2°, about 17.8±0.2°, about 19.7±0.2°, about 20.7±0.2°, about 21.2±0.2°, and about 22.1±0.2°.

TABLE 61
XRPD Diffractogram for Benzenesulfonic Acid Salt Pattern 3

| Pos. [°2θ] | d-spacing [Å] | Rel. Int. [%] |
|---|---|---|
| 5.5469 | 15.9327 | 30.34 |
| 7.2688 | 12.1619 | 11.67 |
| 9.8149 | 9.0120 | 84.22 |
| 10.2082 | 8.6655 | 17.77 |
| 10.7046 | 8.2648 | 10.67 |
| 11.9359 | 7.4149 | 35.99 |
| 12.2201 | 7.2430 | 74.23 |
| 12.6540 | 6.9956 | 19.58 |
| 13.8238 | 6.4062 | 18.47 |
| 14.4655 | 6.1234 | 10.88 |
| 15.7391 | 5.6306 | 18.57 |
| 16.3504 | 5.4215 | 23.46 |
| 16.6796 | 5.3152 | 26.04 |
| 16.9610 | 5.2277 | 27.03 |
| 17.3583 | 5.1089 | 81.38 |
| 17.8036 | 4.9821 | 32.40 |
| 18.3942 | 4.8234 | 26.47 |
| 18.8301 | 4.7128 | 28.89 |
| 19.2510 | 4.6107 | 21.09 |
| 19.7308 | 4.4996 | 100.00 |
| 20.6824 | 4.2947 | 54.25 |
| 21.2342 | 4.1843 | 78.59 |
| 22.0635 | 4.0289 | 34.38 |
| 23.1082 | 3.8459 | 5.45 |
| 24.0707 | 3.6973 | 17.91 |
| 24.7714 | 3.5943 | 25.74 |
| 25.0546 | 3.5543 | 26.41 |
| 25.9696 | 3.4311 | 19.85 |
| 26.4262 | 3.3728 | 26.20 |
| 26.8644 | 3.3188 | 25.92 |
| 27.8509 | 3.2034 | 19.79 |
| 28.1871 | 3.1660 | 13.46 |
| 28.6857 | 3.1121 | 10.89 |
| 29.6027 | 3.0177 | 18.34 |
| 30.2698 | 2.9527 | 12.84 |
| 31.4973 | 2.8404 | 6.01 |

Table 62 below provides the results of the XRPD performed on benzenesulfonic acid salt Pattern 3*. (* indicates slight shifts in XRPD peak positions in comparison with benzenesulfonic acid salt Pattern 3.) The XRPD exhibited sharp peaks, indicating the sample was composed of crystalline material. Significant peaks were observed in the XRPD on benzenesulfonic acid salt Pattern 3* at about 5.7±0.2°, about 9.8±0.2°, about 10.0±0.2°, about 12.0±0.2°, about 12.2±0.2°, about 17.5±0.2°, about 17.9±0.2°, about 19.7±0.2°, about 20.8±0.2°, and about 21.8±0.2°.

TABLE 62
XRPD Peak List for Benzenesulfonic Acid Salt Pattern 3*

| Pos. [°2θ] | d-spacing [Å] | Rel. Int. [%] |
|---|---|---|
| 5.6664 | 15.5969 | 33.66 |
| 7.2615 | 12.1741 | 13.99 |
| 9.8326 | 8.9958 | 93.82 |
| 9.9719 | 8.8704 | 99.01 |
| 10.7623 | 8.2206 | 13.63 |
| 12.0287 | 7.3579 | 55.01 |
| 12.2414 | 7.2305 | 60.24 |
| 12.9256 | 6.8492 | 22.96 |
| 13.8611 | 6.3890 | 12.61 |
| 14.4418 | 6.1334 | 17.38 |
| 15.6728 | 5.6543 | 17.37 |
| 16.0421 | 5.5250 | 14.88 |
| 16.4101 | 5.4019 | 23.95 |
| 16.9706 | 5.2247 | 26.17 |
| 17.5448 | 5.0550 | 84.19 |
| 17.9238 | 4.9490 | 36.51 |
| 18.8067 | 4.7186 | 27.52 |
| 19.7370 | 4.4982 | 100.00 |
| 20.8135 | 4.2679 | 37.22 |
| 21.2056 | 4.1864 | 20.09 |
| 21.7596 | 4.0844 | 48.15 |
| 22.4526 | 3.9599 | 18.13 |
| 24.1305 | 3.6882 | 13.55 |
| 24.6712 | 3.6086 | 14.26 |
| 25.0918 | 3.5491 | 24.02 |
| 25.9732 | 3.4306 | 18.85 |
| 26.6644 | 3.3432 | 22.24 |
| 27.0072 | 3.3016 | 21.99 |
| 27.8504 | 3.2035 | 18.02 |
| 28.2482 | 3.1593 | 12.54 |
| 29.5229 | 3.0257 | 13.05 |
| 30.4214 | 2.9384 | 8.81 |

### Example 11. Aqueous Solubility on Salt Hits

An aqueous solubility assessment was carried out on any salt hits as follows. A 2-5 mg amount of material was added to a 1.5 mL glass vial. Water was added in 100 μL aliquots up to a maximum volume of 500 μL and observations were recorded. The samples were stirred for 18 hours at 25° C. The slurries were filtered by centrifugation. The solutions were analyzed by HPLC to determine the concentration.

### Example 12. Polarized Light Microscopy (PLM)

The presence of crystallinity (birefringence) was determined using an Olympus BX53 microscope, equipped with cross-polarizing lenses and a Motic camera. Images were captured using Motic Images Plus 3.0. All images were recorded using the 20× objective, unless otherwise stated.

### Example 13. Thermogravimetric Analysis/Differential Scanning Calorimetry (TG/DSC)

Approximately 5-10 mg of material was added into a pre-tared open aluminum pan and loaded into a TA Instruments Discovery SDT 650 Auto-Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

### Example 14. Differential Scanning Calorimetry (DSC)

Approximately 1-5 mg of material was weighed into an aluminum DSC pan and sealed non-hermetically with an aluminum lid. The sample pan was then loaded into a TA Instruments Discovery DSC 2500 differential scanning calorimeter equipped with a RC90 cooler. For the initial characterization of Compound I HCl, the sample and reference were heated to 215° C. at a scan rate of 10° C./min and the resulting heat flow response monitored. The sample was re-cooled to 20° C. and then reheated again to 200° C. all at 10° C./min. Nitrogen was used as the purge gas, at a flow rate of 50 cm³/min. For the secondary experiment, the same process was used with an upper temperature limit of 200° C.

### Example 15. Dynamic Vapor Sorption (DVS)

Approximately 10-20 mg of sample was placed into a mesh vapor sorption balance pan and loaded into a DVS Advantage dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined. XRPD analysis was then carried out on any solid retained.

### Example 16. Variable Temperature X-Ray Powder Diffraction (VT-XRPD)

VT-XRPD analysis was carried out on a Philips X'Pert Pro Multipurpose diffractometer equipped with a temperature chamber. The samples were scanned between 4 and 35.99° 20 using Cu K radiation (α1 λ=1.54060 Å; α2=1.54443 Å; β=1.39225 Å; α1:α2 ratio=0.5) running in Bragg-Brentano geometry (step size 0.008° 20) using 40 kV/40 mA generator settings. Measurements were performed up to 240° C.

### Example 17. Variable Humidity X-Ray Powder Diffraction (VH-XRPD)

VH-XRPD analysis was carried out on a Philips X'Pert Pro Multipurpose diffractometer equipped with a temperature chamber. The samples were scanned between 4 and 35.99° 20 using Cu K radiation (α1 λ=1.54060 Å; α2=1.54443 Å; β=1.39225 Å; α1:α2 ratio=0.5) running in Bragg-Brentano geometry (step size 0.008° 20) using 40 kV/40 mA generator settings. Measurements were performed at set values between 0 and 90% RH.

### Example 18. Fischer Coulometric Titration (KF)

Approximately 10-15 mg of solid material was accurately weighed into a vial. The solid was then manually introduced into the titration cell of a Mettler Toledo C30 Compact Titrator. The vial was back-weighed after the addition of the solid and the weight of the added solid entered on the instrument. Titration was initiated once the sample had fully dissolved in the cell. The water content was calculated automatically by the instrument as a percentage and the data printed.

### Example 19. Infrared Spectroscopy (IR)

Infrared spectroscopy was carried out on a Bruker ALPHA P spectrometer. Sufficient material was placed onto the center of the plate of the spectrometer and the spectra were obtained using the following parameters: resolution: 4 cm-1; background scan time: 16 scans; sample scan time: 16 scans; data collection: 4000 to 400 cm-1; result spectrum: transmittance; software: OPUS version 6.

### Example 20. Nuclear Magnetic Resonance (NMR)

NMR experiments were performed on either a Bruker AVA400 spectrometer, a Bruker AVIIIHD spectrometer, or a Bruker Pro500 spectrometer. Experiments were performed in deuterated dimethyl sulfoxide and each sample was prepared to ca. 10 mM concentration.

### Example 21. High Performance Liquid Chromatography

### High Performance Liquid Chromatography-Charged Aerosol Detection (HPLC-CAD)

Column: Phenomenex Aeris Peptide XB-C18 100 Å 150 mm×4.6 mm, 3.5 μm; stainless steel frit guard+Dionex Acclaim Trinity P2 50 mm×2.1 mm, 3 μm. Column temperature: 30° C. Autosampler temperature: ambient. Injection Volume: 4 μL. Flow rate: 0.45 mL/min. Mobile phase A: water. Mobile phase B: 100 mM ammonium formate in H₂O pH 3.65. Detection: CAD. Gain: 100 pA. Offset: 0%. Filter: Corona. Nebulizer temperature: 30° C.

### Gradient Program:

| Time (minutes | Solvent B (%) |
|---|---|
| 0.0 | 10 |
| 1.0 | 10 |
| 11.0 | 20 |
| 12.0 | 20 |
| 12.1 | 90 |
| 15.0 | 90 |
| 15.1 | 10 |
| 23.0 | 10 |

### High Performance Liquid Chromatography-Ultraviolet Detection (HPLC-UV)

Column: Agilent Zorbax SB-C18 150×4.6 mm, 3.5 μm. Column Temperature: 30° C. Autosampler Temperature: ambient. UV wavelength: 254 nm. Injection Volume: 1 μL. Flow Rate: 0.8 mL/min. Mobile phase A: 0.02% TFA in deionized water. Mobile phase B: methanol.

### Gradient Program:

| Time (minutes) | Solvent B (%) |
|---|---|
| 0.0 | 10 |
| 0.5 | 10 |
| 10.0 | 90 |
| 20 | 90 |
| 20.1 | 10 |
| 27.0 | 10 |

### Example 22. Characterization of Hits from Sulfuric Acid

The results of the characterization of the hits with sulfuric acid are presented in Table 63.

TABLE 63
Results of Characterization of Sulfuric Acid

| | | DSC - | Solvent | Solubility in |
|---|---|---|---|---|
| XRPD | | Thermal | according to ¹H | water at 25° C. |
| Pattern | TG - Mass Loss | Events | NMR | (mg/mL) |
| Sulfate | 5.2 wt. % up to 91.5° C. | Broad | residual | 0.031 |
| Pattern 1 | 3.4 wt. % up to 250° C. | endothermic | 2-propanol | |
| | | event with | | |
| | | onset at | | |
| | | 211.1° C. | | |
| Sulfate | 4.2 wt. % up to 219.7° C. | Endothermic | 0.4 eq. of | 0.113 |
| Pattern 2 | | event onset | acetone | |
| | | with onset at | | |
| | | 241.9° C. | | |
| Sulfate | 2.3 wt. % up to 59.0° C. | N/A | 0.6 eq. of | N/A |
| Pattern 3 | | | acetonitrile | |
| Sulfate | 4.3 wt. % up to 166.7° C. | Broad | residual ethanol | 0.051 |
| Pattern 4 | | endothermic | | |
| | | event with | | |
| | | onset of | | |
| | | 229.6° C. | | |
| Sulfate | 4.7 wt. % up to 237.8° C. | Small | 0.6 eq. of ethyl | 0.372 |
| Pattern 5 | | endothermic | acetate | |
| | | event with | | |
| | | onset of | | |
| | | 185.6° C. | | |
| Sulfate | 0.8 wt. % up to 102.4° C. | N/A | residual THF | 0.181 |
| Pattern 6 | | | | |
| Sulfate | 4.2 wt. % up to 223.3° C. | Weak | residual ethyl | 0.046 |
| Pattern 7 | | endothermic | acetate | |
| | | event at | | |
| | | 247.9° C. | | |

Characterization of sulfate Pattern 1 indicated that the material contained residual quantities of 2-propanol. The aqueous solubility of material at 25° C. was determined to be 0.031 mg/mL.

Characterization of sulfate Pattern 2 material indicated that the material contained 0.4 eq. of acetone. The aqueous solubility of the material was determined to be 0.113 mg/mL.

Characterization of sulfate Pattern 3 indicated that the material contained 0.6 eq. of acetonitrile.

Characterization of sulfate Pattern 4 indicated that the material contained residual quantities of ethanol. The aqueous solubility of the material at 25° C. was determined to be 0.051 mg/mL.

Characterization of sulfate Pattern 5 indicated that the material contained 0.6 eq. of ethyl acetate. The aqueous solubility of the material at 25° C. was determined to be 0.372 mg/mL.

Characterization of sulfate Pattern 6 material indicated the material to contain residual quantities of tetrahydrofuran. The aqueous solubility of the material at 25° C. was determined to be 0.181 mg/mL.

Characterization of sulfate Pattern 7 after storage at 40° C./75% RH indicated that the material contained residual quantities of ethyl acetate. The aqueous solubility of the material at 25° C. was determined to be 0.046 mg/mL.

### Example 23. Characterization of Methanesulfonic Acid

The results of the characterization of methanesulfonic acid are presented in Table 64.

TABLE 64
Results of Characterization of Methanesulfonic Acid

| | | DSC - | Solvent | Solubility in |
|---|---|---|---|---|
| XRPD | | Thermal | according to ¹H | water at 25° C. |
| Pattern | TG - Mass Loss | Events | NMR | (mg/mL) |
| Mesylate | 2.4 wt. % up to 63.6° C. | Endothermic | residual 2- | 0.079 |
| Pattern 1 | 3.4 wt. % up to 281.6° C. | event with | propanol | |
| | | onset at | | |
| | | 229.4° C. | | |
| Mesylate | 1.6 wt. % up to 76.1° C. | Endothermic | 0.8 eq. of | 0.569 |
| Pattern 2 | 5.2 wt. % up to 187.2° C. | event with | acetone | |
| | | onset at | | |
| | | 220.8° C. | | |
| | | Broad | | |
| | | endothermic | | |
| | | event with | | |
| | | onset at | | |
| | | 242.6° C. | | |
| Mesylate | 3.5 wt. % up to 197.5° C. | Endothermic | 0.2 eq. of | 0.185 |
| Pattern 3 | 1.6 wt. % up to 266.3° C. | event with | acetonitrile | |
| | | onset at | | |
| | | 247.6° C. | | |
| Mesylate | 1 wt. % up to 67.9° C. | Endothermic | 0.8 eq. of | 0.090 |
| Pattern 4 | | event with | ethanol | |
| | | onset at | | |
| | | 173.8° C. | | |
| | | Endothermic | | |
| | | event with | | |
| | | onset at | | |
| | | 224.0° C. | | |
| | | Endothermic | | |
| | | event with | | |
| | | onset at | | |
| | | 265.5° C. | | |

Characterization of mesylate Pattern 1 indicated the material was a mono-salt (1 eq. of counterion) which contained trace quantities of 2-propanol. The aqueous solubility of the material at 25° C. was determined to be 0.079 mg/mL.

Characterization of mesylate Pattern 2 indicated the material was likely a mono-salt (1.2 eq. of counterion). The material was found to contain 0.8 eq. of acetone prior to storage at 40° C./75% RH. The aqueous solubility of the material at 25° C. was determined to be 0.569 mg/mL.

Characterization of mesylate Pattern 3 indicated the material was a mono-salt (1 eq. of counterion). The material was found to contain 0.2 eq. of acetonitrile prior to storage at 40° C./75% RH. The aqueous solubility of the material at 25° C. was determined to be 0.185 mg/mL.

Characterization of mesylate Pattern 4 indicated the material to be a mono-salt (1 eq. of counterion). The material was found to contain 0.8 eq. of ethanol prior to storage at 40° C./75% 5 RH. The aqueous solubility of the material at 25° C. was determined to be 0.09 mg/mL.

### Example 24. Characterization of Maleic Acid

The results of the characterization of maleic acid are presented in Table 65.

TABLE 65
Results from Maleic Acid

| | | DSC - | Solvent | Solubility in |
|---|---|---|---|---|
| XRPD | | Thermal | according to ¹H | water at 25° C. |
| Pattern | TG - Mass Loss | Events | NMR | (mg/mL) |
| Maleate | 4 wt. % up to 78.1° C. | Endothermic | residual 2- | 0.008 |
| Pattern 1 | | event with | propanol | |
| | | onset at | | |
| | | 167.7° C. | | |
| Maleate | 10.7 wt. % up to 203° C. | Broad | residual acetone | 0.037 |
| Pattern 2 | | endothermic | | |
| | | event with | | |
| | | onset at | | |
| | | 190.7° C. | | |
| Maleate | 14.1 wt. % up to | Broad | 0.5 eq. of | 0.013 |
| Pattern 3 | 215.8° C. | endothermic | ethanol | |
| | | event with | | |
| | | onset at | | |
| | | 165.9° C. | | |
| Maleate | 2 wt. % up to 78.3° C. | Broad | 0.1 eq. of ethyl | n/a |
| Pattern 4 | | endothermic | acetate | |
| | | event with | | |
| | | onset at | | |
| | | 180.8° C. | | |
| Maleate | 5.2 wt. % up to 154.4° C. | Small | 0.4 eq. of THF | n/a |
| Pattern 5 | | endothermic | | |
| | | event with | | |
| | | onset at | | |
| | | 153.2° C. | | |
| | | Small | | |
| | | endothermic | | |
| | | event with | | |
| | | onset at | | |
| | | 166.0° C. | | |

Characterization of maleate Pattern 1 indicated that the material was a mono-salt (1 eq. of counterion). The material was found to contain trace quantities of 2-propnaol. The aqueous solubility of the material at 25° C. was determined to be 0.008 mg/mL.

Characterization of maleate Pattern 2 indicated that the material was a mono-salt (1 eq. of counterion). The material was found to contain trace quantities of acetone. The aqueous solubility of the material at 25° C. was determined to be 0.037 mg/mL.

Characterization of maleate Pattern 3 material indicated that the material was likely a mono-salt (0.9 eq. of counterion). The material was found to contain 0.5 eq. of ethanol prior to storage at 40° C./75% RH. The aqueous solubility of the material at 25° C. was determined to be 0.013 mg/mL.

Characterization of maleate Pattern 4 indicated that the material was likely a mono-salt (1.2 eq. of counterion). The material was found to contain 0.1 eq. of ethyl acetate prior to storage at 40° C./75% RH.

Characterization of maleate Pattern 5 indicated that the material was likely a mono-salt (0.9 eq. of counterion). The material was found to contain 0.4 eq. of tetrahydrofuran prior to storage at 40° C./75% RH.

### Example 25. Characterization of Phosphoric Acid

The results of the characterization of phosphoric acid are presented in Table 66.

TABLE 66
Results of Characterization Phosphoric Acid

| | | | | Solubility |
|---|---|---|---|---|
| | | DSC - | Solvent | in water |
| XRPD | TG - Mass | Thermal | according | at 25° C. |
| Pattern | Loss | Events | to ¹H NMR | (mg/mL) |
| Phosphate | 1.7 wt. % up to | N/A | residual | 0.249 |
| Pattern 1 | 89.1° C. | | acetone | |
| Phosphate | 1.2 wt. % up to | N/A | 0.3 eq. of | 0.272 |
| Pattern 2 | 160.3° C. | | acetonitrile | |
| Phosphate | 5.1 wt. % up to | Small | 0.1 eq. of ethyl | 0.275 |
| Pattern 3 | 282.1° C. | endothermic | acetate | |
| | | event with | | |
| | | onset at | | |
| | | 170.3° C. | | |
| | | Small | | |
| | | endothermic | | |
| | | event with | | |
| | | onset at | | |
| | | 229.2° C. | | |
| Phosphate | 2.4 wt. % up to | N/A | 0.3 eq. of THF | 0.206 |
| Pattern 4 | 142.9° C. | | | |
| Phosphate | 3.0 wt. % up to | Broad | residual | 0.0354 |
| Pattern 2* | 82.1° C. | endothermic | acetonitrile | |
| | | event with | | |
| | | onset at | | |
| | | 178.6° C. | | |
| Phosphate | 3.8 wt. % up to | Small | residual ethyl | 0.0987 |
| Pattern 7* | 283.1° C. | endothermic | acetate | |
| | | event with | | |
| | | onset at | | |
| | | 172.3° C. | | |
| Phosphate | 2.5 wt. % up to | Weak | 0.1 eq. of THF | 0.0805 |
| Pattern 8* | 98.8° C. | endothermic | | |
| | | events at ca. | | |
| | | 178.5° C. and | | |
| | | 229.0° C. | | |

Characterization of phosphate Pattern 1 indicated that the material contained residual quantities of acetone. The aqueous solubility of the material at 25° C. was determined to be 0.249 mg/mL.

Characterization of phosphate Pattern 2 material indicated that the material contained 0.3 eq. of acetonitrile prior to storage of the material at 40° C./75% RH. The aqueous solubility of the material at 25° C. was determined to be 0.272 mg/mL.

Characterization of phosphate Pattern 3 indicated that the material contained 0.1 eq. of ethyl acetate prior to storage of the material at 40° C./75% RH. The aqueous solubility of the material at 25° C. was determined to be 0.275 mg/mL.

Characterization of phosphate Pattern 4 indicated that the material contained 0.3 eq. of THE prior to storage at 40° C./75% RH. The aqueous solubility of the material at 25° C. was determined to be 0.206 mg/mL.

Characterization of phosphate Pattern 6 indicated the formation of amorphous material after storage at 40° C./75% RH therefore pattern 6 was not considered a stable form.

Characterization of phosphate Pattern 2* indicated the material to contain trace quantities of acetonitrile. The aqueous solubility of the material at 25° C. was determined to be 0.0354 mg/mL.

Characterization of Pattern 7* indicated that the material contained trace quantities of ethyl acetate. The aqueous solubility of the material at 25° C. was determined to be 0.0987 mg/mL.

Characterization of Pattern 8* indicated that the material contained 0.1 eq. of THF. The aqueous solubility of the material at 25° C. was determined to be 0.0805 mg/mL.

### Example 26. Characterization of (+)-L-Tartaric Acid

The results of the characterization of (+)-L-tartaric acid are presented in Table 67.

TABLE 67
Results of Characterization of (+)-L-Tartaric Acid

| | | | | Solubility |
|---|---|---|---|---|
| | | DSC - | Solvent | in water |
| XRPD | TG - Mass | Thermal | according | at 25° C. |
| Pattern | Loss | Events | to ¹H NMR | (mg/mL) |
| Tartrate | 0.6 wt. % up to | Endothermic | residual 2- | 0.003 |
| Pattern 1 | 216.4° C. | event with | propanol | |
| | 12.5 wt. % up to | onset at | | |
| | 284.5° C. | 261.0° C. | | |
| Tartrate | 0.5 wt. % up to | Broad | 0.9 eq. of | 0.080 |
| Pattern 2 | 151.3° C. | endothermic | acetone | |
| | 2.9 wt. % up to | event with | | |
| | 183.6° C. | onset at | | |
| | 11.2 wt. % up to | 187.6° C. | | |
| | 255.4° C. | | | |

Characterization of tartrate Pattern 1 indicated that the material was a mono-salt (1 eq. of counterion). The material was found to contain trace quantities of 2-propanol. The aqueous solubility of the material at 25° C. was determined to be 0.003 mg/mL.

Characterization of tartrate Pattern 2 indicated the material was likely a hemi-salt (0.5 eq. of counterion). The material was found to contain 0.9 eq. of acetone prior to storage at 40° C./75% RH. The aqueous solubility of the material at 25° C. was determined to be 0.080 mg/mL.

### Example 27. Characterization of Citric Acid

The results of the characterization of citric acid are presented in Table 68.

TABLE 68
Results of Characterization of Citric Acid

| | | | | Solubility |
|---|---|---|---|---|
| | | DSC - | Solvent | in water |
| XRPD | TG - Mass | Thermal | according | at 25° C. |
| Pattern | Loss | Events | to ¹H NMR | (mg/mL) |
| Citrate | 0.4 wt. % up to | Broad | 0.1 eq. of | 0.057 |
| Pattern 1 | 171° C. | endothermic | acetone | |
| | | event with | | |
| | | onset at | | |
| | | 181.8° C. | | |
| Citrate | 23.5 wt. % up to | Small | 0.4 eq. of | 0.023 |
| Pattern 2 | 237.7° C. | endothermic | acetonitrile | |
| | | event with | | |
| | | onset at | | |
| | | 155.9° C. | | |
| Citrate | 2.8 wt. % up to | No definable | 0.1 eq. of | 0.034 |
| Pattern 3 | 144.1° C. | events | THF | |

Characterization of citrate Pattern 1 indicated the material to be a mono-salt (1 eq. of counterion). The material was found to contain 0.1 eq. of acetone prior to storage at 40° C./75% RH. The aqueous solubility of the material at 25° C. was determined to be 0.057 mg/mL.

Characterization of citrate Pattern 2 indicated the material to be a mono-salt (1 eq. of counterion). The material was found to contain 0.4 eq. of acetonitrile prior to storage at 40° C./75% RH. The aqueous solubility of the material at 25° C. was determined to be 0.023 mg/mL.

Characterization of citrate Pattern 3 indicated the material was likely a hemi-salt (0.5 eq. of counterion). The material was found to contain 0.1 eq. of THE prior to storage at 40° C./75% RH. The aqueous solubility of the material at 25° C. was determined to be 0.034 mg/mL.

### Example 28. Characterization of Hydrobromic Acid

The results of the characterization of hydrobromic acid are presented in Table 69.

TABLE 69
Results of Characterization of Hydrobromic Acid

| | | | | Solubility |
|---|---|---|---|---|
| | | DSC - | Solvent | in water |
| XRPD | TG - Mass | Thermal | according | at 25° C. |
| Pattern | Loss | Events | to ¹H NMR | (mg/mL) |
| HBr | 0.8 wt. % up to | Small | 0.1 eq. of 2- | 0.010 |
| Pattern 1 | 69.9° C. | endothermic | propnaol | |
| | | event with | | |
| | | onset at | | |
| | | 307.6° C. | | |
| HBr | 1.2 wt. % up to | Small | residual | 0.031 |
| Pattern 2 | 242.4° C. | endothermic | acetone | |
| | | event with | | |
| | | onset at | | |
| | | 284.2° C. | | |

Characterization of HBr Pattern 1 indicated the material to contain 0.1 eq. of 2-propanol. The aqueous solubility of the material at 25° C. was determined to be 0.010 mg/mL.

Characterization of HBr Pattern 2 indicated the material to contain trace quantities of acetone. The aqueous solubility of the material at 25° C. was determined to be 0.031 mg/mL.

### Example 29. Characterization of Benzenesulphonic Acid

The results of the characterization of benzenesulphonic acid are presented in Table 70.

TABLE 70
Results of Characterization of Benzenesulphonic Acid

| | | | | Solubility |
|---|---|---|---|---|
| | | DSC - | Solvent | in water |
| XRPD | TG - Mass | Thermal | according | at 25° C. |
| Pattern | Loss | Events | to ¹H NMR | (mg/mL) |
| Besylate | 3.8 wt. % up to | Broad | residual 2- | 0.026 |
| Pattern 1 | 111.1° C. | endothermic | propanol | |
| | | event with | | |
| | | onset at | | |
| | | 163.1° C. | | |
| Besylate | 3.0 wt. % up to | Large | 0.5 eq. of | 0.076 |
| Pattern 2 | 209.0° C. | exothermic | acetone | |
| | | event with | | |
| | | onset at | | |
| | | 253.5° C. | | |
| Besylate | 2.5 wt. % up to | Weak | residual | 0.039 |
| Pattern 3 | 244.2° C. | exothermic | THF | |
| | | event at | | |
| | | 173.3° C. | | |

Characterization of besylate Pattern 1 indicated that the material was a mono-salt (1 eq. of counterion). The material was found to contain trace amounts of 2-propanol. The aqueous solubility of the material at 25° C. was determined to be 0.026 mg/mL.

Characterization of besylate Pattern 2 indicated the material was likely a mono-salt (1.3 eq. of counterion). The material was found to contain 0.5 eq. of acetone prior to drying at 40° C./75% RH. The aqueous solubility of the material at 25° C. was determined to be 0.076 mg/mL.

Characterization of besylate Pattern 3 indicated that the material was a mono-salt (1 eq. of counterion). The material was found to contain trace quantities of tetrahydrofuran. The aqueous solubility of the material at 25° C. was determined to be 0.039 mg/mL.

### Example 30. Characterization of Hydrochloric Acid

The following results were obtained from characterization of HCl Pattern 1.

XRPD analysis revealed that the material was crystalline.

PLM analysis revealed that the material had a small particle size, with block-like birefringent crystals.

TG/DSC: The TGA trace showed an initial mass loss of 4.2 wt. % between the onset of heating and 106.7° C. (1.1 eq. of water). A second mass loss of 7.8 wt. % occurred between 182.1 and 285.5° C. (1.1 eq. of HCl): Simultaneous DSC analysis showed an initial broad endothermic event concomitant with the initial mass loss with an onset of 51.8° C., and peak at 85.2° C. A small exothermic event was observed with the onset of the second mass loss, onset of 193.8° C., and peak at 201.9° C. Two endothermic events concomitant with the second mass loss. The first endothermic event was broad with an onset of 251.3° C., and peak at 263.0° C. The second smaller endothermic event had an onset of 275.6° C., and a peak at 279° C.

DSC: The first heat cycle revealed two endothermic events. The first endothermic event: onset at 34.4° C., peak at 67.6° C. Second endothermic event: onset at 177.1° C., peak at 192°.

VT-XRPD analysis (Table 71) correlated with the events observed in the TG/DSC analysis. No form change was observed after the initial mass loss up to 120° C., suggesting this mass loss to be associated with the loss of water from the material. A form change was observed at 185° C. prior to the exothermic event observed in the DSC trace of the TG/DSC analysis. A mixture of forms is observed at 220° C. prior to the broad endothermic event identified in the TG/DSC analysis consistent with the onset of counterion loss. Above this temperature VT-XRPD analysis revealed the presence of free base and confirmed the final endothermic event at 275.6° C. as a melt. After this temperature the material was amorphous and did not re-crystallize, forming a black solid.

TABLE 71
Results of VT-XRPD Analysis of HCl Pattern 1

| | Heating Rate | |
|---|---|---|
| Temperature | to Next Step | |
| (° C.) | (° C./min) | XRPD |
| 30 | 10 | Crystalline |
| 120 | 10 | Crystalline |
| 165 | 10 | Crystalline |
| 190 | 10 | Crystalline |
| 240 | 10 | Amorphous |
| 30 | 10 | Amorphous |

DVS analysis revealed the material to be moderately hygroscopic with an uptake of 5.15% and 5.10% in the first and second sorption cycles respectively. Analysis of the material post DVS by XRPD indicated no observable form change had occurred.

KF analysis revealed a water content of 4.2%. This value was consistent with the initial mass loss observed in the TG/DSC analysis.

HPLC-CAD analysis revealed that HCl Pattern 1 to contained 7.7% w/w chloride.

The results of the characterization of HCl Pattern 2 are presented in Table 72.

TABLE 72
Results of Characterization of Primary HCl Pattern 2

| | | | | Solubility |
|---|---|---|---|---|
| | | DSC - | Solvent | in water |
| XRPD | TG - Mass | Thermal | according | at 25° C. |
| Pattern | Loss | Events | to ¹H NMR | (mg/mL) |
| HCl | 1.1 wt. % up to | Weak | residual | 0.0099 |
| Pattern 2 | 60.2° C. | endothermic | ethanol | |
| | 6.1 wt. % up | event with | | |
| | 255.3° C. | onset ca. | | |
| | | 242.1° C. | | |
| | | Endothermic | | |
| | | event with | | |
| | | onset at | | |
| | | 276.9° C. | | |

Characterization of HCl Pattern 2 indicated the material was likely a mono-HCl salt, as described in Example 25. The material was found to contain trace quantities of ethanol before and after storage at 40° C./75% RH. The aqueous solubility of the material at 25° C. was determined to be 0.0099 mg/mL.

### Example 31. Assessment of Solvate Formation or Form Change of Hydrochloride Pattern 1 in Ethanol

HCl Pattern 1 was stirred in ethanol to assess any solvation or form change in the solvent system. The following procedure was carried out. HCl Pattern 1 (20 mg) was suspended in 1 mL of ethanol. The suspension was thermally cycled for three days according to the following cycle:
Heat to 40° C. at a rate of 0.1° C./min;
Hold at 40° C. for one hour;
Cool to 5° C. at a rate of 0.1° C./min;
Hold at 5° C. for one hour.

After three days, observations were recorded, and solids were isolated by centrifugation. Wet solids were analyzed by XRPD.

The XRPD analysis indicated that thermal cycling of HCl Pattern 1 in ethanol produced HCl Pattern 2. TG/DSC and ¹H NMR spectroscopy revealed that only trace quantities of ethanol were present in the material. HCl Pattern 2 was observed from slurry of HCl Pattern 1 in ethanol, and salt experiments using both one and two equivalents of HCl, therefore HCl Pattern 2 was likely a mono HCl salt form.

### Example 32. 7-Day Indicative Stability of HCl Pattern 1

The stability of HCl Pattern 1 was determined using the following procedure. HCl Pattern 1 (30 mg) was weighed into 3×2 mL glass vials. The three vials were then stored under the following conditions for 7-days:
Ambient temperature, humidity and light (open vial);
40° C./75% RH (open vial);
80° C. (closed vial).

After 7 days, each sample was analyzed by: XRPD to assess form changes and HPLC to determine the purity of the samples.

The results of the 7-day indicative stability assessment of are presented in Table 73. There was no observable change in form, however a decrease in purity was noted for all samples, with the largest increase in impurities coming after storage at 80° C.

TABLE 73
Results and Observations for the 7-day Indicative
Stability Assessment of HCl Pattern 1

| | | | HPLC |
|---|---|---|---|
| | | | Purity |
| Condition | Observation | XRPD | (% w/w) |
| Ambient | Yellow Solid | HCl Pattern 1 | 98.02 |
| 40° C. / 75% RH | White Solid | HCl Pattern 1 | 97.98 |
| 80° C. | White Solid | HCl Pattern 1 | 96.97 |

### Example 33. Thermodynamic Solubility of HCl Pattern 1

The thermodynamic solubility of HCl Pattern 1 was determined in duplicate according to the following procedure. HCl Pattern 1 (20 mg) was weighed into 6×2 mL glass vials. Then, 1000 μL of pH 1 hydrochloride, pH 4.5 acetate and pH 6.8 phosphate buffers were added to individual vials in 100 μL aliquots to give white slurries at each pH value. The pH of each sample was recorded followed by equilibration (stirring at 25° C. for 24 hours). The pH was re-recorded and adjusted where necessary. The samples were isolated by centrifuge filtration. The wet solids were isolated and analyzed by XRPD prior to drying at 40° C. under vacuum for ca. 18 hours. The concentrations of the saturated solutions were determined by HPLC.

The results of the thermodynamic solubility analysis are presented in Table 74. At pH 4.5 and 6.8, XRPD analysis revealed the formation of free base.

TABLE 74
Results and Observations for the Thermodynamic Solubility Assessment of HCl Pattern

| | Observations | | pH | | Solubility | |
|---|---|---|---|---|---|---|
| Buffer System | Initial | 1 Day | Initial | 1 Day | (mg/mL) | XRPD |
| pH 1 (HCl) | WS | WS | 0.98 | 1.02 | 0.0038 | 1 |
| pH 1 (HCl) | WS | WS | 0.94 | 1.00 | 0.0027 | 1 |
| pH 4.5 (Acetate) | WS | WS | 4.54 | 4.43 | <LOQ | 1+ |
| pH 4.5 (Acetate) | WS | WS | 4.55 | 4.45 | <LOQ | 1+ |
| pH 6.8 | WS | WS | 6.58 | 6.83 | <LOQ | 1+ |
| (Phosphate) | | | | | | |
| pH 6.8 | WS | WS | 6.70 | 6.84 | <LOQ | 1+ |
| (Phosphate) | | | | | | |

### Example 34. Preparation of Sulfate Pattern 7

Sulfate Pattern 7 was prepared as follows.

Free base (100 mg) was suspended in 5 mL of ethyl acetate at ambient temperature. Then, 13.4 μL of sulfuric acid was added directly to the suspension of free base. The experiment was thermally cycled as follows:
40° C. to 5° C. at a rate of 0.1° C./min;
1 h at 5° C.;
5 to 40° C. at a rate of 0.1° C./min;
1 h at 40° C.

The suspension was thermally cycled for three days and yielded a white slurry. The slurry was isolated by Buchner filtration. The cake was washed with ca. 2 mL of ethyl acetate. The solids were isolated and dried at 40° C. under vacuum for ca. 24 hours. The solids were furthered dried at 40° C./75% RH.

The following results were obtained from the experiment and characterization of sulfate pattern obtained.

XRPD analysis indicated that the material was not consistent with the Sulfate Pattern 5 material isolated from the primary salt experiments. XRPD analysis indicated a new diffraction pattern which was designated Sulfate Pattern 7.

TG/DSC: The TGA trace showed an initial mass loss of 1.7 wt. % between the onset of heating and 78° C. (0.5 eq. of water or 0.1 eq. ethyl acetate). A second mass loss of 3.5 wt. % was observed between 122.1 and 234.1° C. (1.1 eq. of water or 0.2 eq. ethyl acetate). The DSC trace showed a weak endothermic event at 247.9° C. which occurred during the onset of degradation.

As no definable thermal events were observed prior to degradation of the material no stand-alone DSC measurements were carried out on Sulfate Pattern 7.

¹H NMR analysis of Sulfate Pattern 7 indicated a shift in key resonances consistent with salt formation. Based on the ratio of selected integrals, trace quantities of ethyl acetate were present within the Sulfate Pattern 7 material.

The aqueous solubility of Sulfate Pattern 7 at 25° C. was determined by HPLC to be 0.046 mg/mL.

### Example 35. Preparation of Phosphate Pattern 7*

The following procedure was carried out for the scale up of Phosphate Pattern 7. Upon scale up of the reaction, Pattern 7* was obtained showing only minor difference to that of pattern 7.

Free base (500 mg) was suspended in 15 mL of ethyl acetate at ambient temperature. Then, 83.8 μL of phosphoric acid was added directly to the suspension of free base. The experiment was thermally cycled as follows:
40° C. to 5° C. at a rate of 0.1° C./min;
1 h at 5° C.;
5 to 40° C. at a rate of 0.1° C./min;
1 h at 40° C.

The suspension was thermally cycled for two days and yielded a white slurry. The slurry was isolated by Buchner filtration. The cake was washed with ca. 5 mL of ethyl acetate. The solids were isolated and dried at 40° C. under vacuum for ca. 24 hours. The solids were furthered dried at 40° C./75% RH.

### Example 36. 7-Day Stability Assessment of Phosphate Pattern 7*

The stability of Phosphate Pattern 7* was determined using the following procedure. Phosphate Pattern 7* (20 mg) was stored under the following conditions:
Ambient temperature, light and humidity (open vial);
40° C./75% RH (open vial);
80° C. (closed vial).

After 7 days at each storage condition, the solids were analyzed by XRPD to assess any changes in solid form and by HPLC to determine purity.

The results of the 7-day indicative stability assessment of are presented in Table 75. There was no observable change in form, however a significant decrease in purity was observed after storage of the material at 40° C./75% RH.

TABLE 75
Results and Observations for the 7-day Indicative
Stability Assessment of Phosphate Pattern 7*

| | | | HPLC |
|---|---|---|---|
| | | | Purity |
| Condition | Observation | XRPD | (% w/w) |
| Ambient | Faint Yellow Solid | Phosphate Pattern 7* | 97.87 |
| 40° C. / | Faint Pink Solid | Phosphate Pattern 7* | 89.63 |
| 75% RH | | | |
| 80° C. | White Solid | Phosphate Pattern 7* | 98.08 |

### Example 37. Thermodynamic Solubility Study of Phosphate Pattern 7*

The solubility study of Phosphate Pattern 7* was carried out using the following procedure. Approximately 10 mg of Phosphate Pattern 7* was suspended in 1 mL of one of the following buffer systems: pH 1 HCl buffer, pH 4.5 acetate buffer, and pH 6.8 phosphate buffer. Buffer systems were added in 250 μL aliquots up to a total volume of 1 mL. The pH of each slurry was recorded and adjustments were made to the pH 1 system. No initial adjustment was made to the pH 4.5 or 6.8 system. An aliquot of the slurry was analyzed by XRPD. The slurries were stirred for ca. 24 hours at 25° C. The pH was checked and adjusted where required. The slurries were filtered by centrifugation. The solids were isolated and analyzed by XRPD. The solutions were analyzed by HPLC to determine concentration.

The results of the thermodynamic solubility analysis of Phosphate Pattern 7* are presented in Table 76. Phosphate Pattern 7* was found to disproportionate in three buffer systems utilized giving a low aqueous solubility. At pH 1, the XRPD analysis of the residual solids revealed a mixture of HCl Pattern 1 and free base. At pH 4.5 and 6.8, XRPD analysis of the residual solids revealed the dissociation to free base.

TABLE 76
Results and Observations for the Thermodynamic Solubility Assessment of Phosphate
Pattern 7*

| | Observations | | pH | | Solubility | |
|---|---|---|---|---|---|---|
| Buffer System | Initial | 1 Day | Initial | 1 Day | (mg/mL) | XRPD |
| pH 1 (HCl) | FYS | FYS | 0.99 | 1.08 | 0.0038 | HCl Pattern 1 |
| pH 1 (HCl) | FYS | FYS | 0.99 | 1.04 | 0.0027 | HCl Pattern 1 |
| pH 4.5 (Acetate) | WS | WS | 2.88 | 4.43 | 0.0052 | Free base |
| pH 4.5 (Acetate) | WS | WS | 2.96 | 4.42 | 0.0058 | Free base |
| pH 6.8 | WS | WS | 1.66 | 6.88 | 0.0042 | Free base |
| (Phosphate) | | | | | | |
| pH 6.8 | WS | WS | 1.55 | 6.72 | 0.0032 | Free base |
| (Phosphate) | | | | | | |

### Example 38. Disproportionation Study of Phosphate Pattern 7*

A study disproportionation was carried out using the following procedure. Approximately 10 mg of Phosphate Pattern 7* was suspended in 1 mL of water. The pH was recorded, and the suspension was stirred for ca. 24 hours at 25° C. After 24 hours the pH was recorded, and the slurry was filtered by centrifugation. The solids were isolated and analyzed by XRPD. The solution was isolated and analyzed by HPLC to determine concentration.

The disproportionation study of Phosphate Pattern 7* revealed a form change. XRPD analysis revealed the material to be poorly crystalline with a diffraction pattern similar to that of free base, indicative of disproportionation.

### Example 39. Hydration Study of Phosphate Pattern 7*

A hydration study was carried out on the material according to the following procedure. Phosphate Pattern 7* material (10 mg) was suspended in an ethanol: water mixture of known water activity. Ethanol: water solutions (Table 77) were added in 250 μL aliquots to a total volume of 1 mL. The slurries were stirred at 25° C. for 24 hours. After 24 hours the slurries were filtered by centrifugation. The solids were isolated and analyzed by XRPD. The solution was isolated and analyzed by HPLC to determine concentration.

TABLE 77
Experimental Details of Salt Hydration Study

| | | | Solvent |
|---|---|---|---|
| | | Ethanol:Water | Volume |
| Input | Calc Aw | (% v/v) | (mL) |
| Phosphate Pattern 7* | 0.257 | 98.4:1.6 | 1 |
| Phosphate Pattern 7* | 0.507 | 92.7:7.1 | 1 |
| Phosphate Pattern 7* | 0.745 | 63.6:36.4 | 1 |

The results of the hydration study of Phosphate Pattern 7* (Table 78) indicate disproportionation of the salt to free base material across all of the ethanol: water mixtures.

TABLE 78
Results and Observation for the Hydration
Study of Phosphate Pattern 7*

| Solvent System | Calc | Observations | | Solubility | |
|---|---|---|---|---|---|
| (% v/v) | Aw | Initial | 1 Day | (mg/mL) | XRPD |
| Ethanol:Water | 0.257 | WS | WS | 1.2170 | Free base |
| (98.4:1.6) | | | | | |
| Ethanol:Water | 0.507 | WS | WS | 1.6648 | Free base |
| (92.9:7.1) | | | | | |
| Ethanol:Water | 0.745 | WS | WS | 0.9878 | Free base |
| (63.6:36.4) | | | | | |

The following results were obtained from the scale up and characterization of Phosphate Pattern 7*.

The XRPD analysis indicated that the material was consistent with Phosphate Pattern 7* Some small peak shifts were observed on drying at 40° C. under vacuum and after storage at 40° C./75% RH.

PLM analysis indicated the material to have a small particle size with a block-like morphology. The crystalline material was poorly birefringent and was aggregated.

FT-IR analysis indicated that water was present.

TG/DSC: The TGA trace revealed an initial mass loss of 1.9 wt. % between the onset of heating and 79.5° C. (0.6 eq. of water or 0.1 eq. of ethyl acetate). A second gradual mass loss of 1.8 wt. % was observed between 162.7 and 238.6° C. (0.5 eq. of water, 0.1 eq. of ethyl acetate or 0.1 eq. of phosphoric acid. The DSC trace revealed a small endothermic event concomitant with the onset of the second mass loss; with an onset of 173.4° C., and peak at 178.3° C.

DSC: The first heat cycle of the DSC revealed a broad endothermic event with onset and peak temperatures of 170.0 and 179.3° C. respectively. Two weak endothermic events were also observed at 208.6 and 229.6° C.

KF analysis of Phosphate Pattern 7* indicated a moisture content of 3.1 wt. %.

VT-XRPD correlated with the events observed in the TG/DSC analysis. No form change was observed up to 165° C. indicating that the initial mass loss observed in the TGA trace up to a 79.5° C. was a loss of unbound water. A loss of crystallinity was observed at 190° C. and the material was predominantly amorphous. At 240° C., the temperature identified as the end of the second mass loss in the TGA trace, the material was found to be completely amorphous and did not crystallize when cooled, forming a black solid.

DVS analysis revealed that the material was moderately hygroscopic with an uptake of 4.76% and 5.18% in the first and second sorption cycles respectively. Analysis of the material post DVS by XRPD indicated no observable form change had occurred.

VH-XRPD correlated with the DVS analysis, with no form change observed at elevated RH (90% RH) or at low RH (2% RH). Slight shifts in peak positions were observed over the range of RH.

The ¹H NMR analysis revealed a shift in a key resonance when compared with free base which was indicative of salt formation.

HPLC analysis indicated that the material had a purity value of 97.87% (% area purity).

HPLC-CAD indicated a phosphate content of 12.4% w/w.

TABLE 79
Results Summary of the Characterization of Phosphate Pattern 7*

| Analysis | | | Phosphate Pattern 7* |
|---|---|---|---|
| Solvent System | | | Ethyl Acetate |
| XRPD | Wet | | Pattern 7* |
| | Dry | | Pattern 7* |
| | 40° C. / 75% RH | | Pattern 7* |
| PLM | Morphology | | Aggregated small block-like crystals |
| FT-IR | | | Consistent with structure |
| TG/DSC | TG | | 1.9 wt. % up to 79.5° C. (0.6 eq. water); |
| | | | 1.8 wt. % up to 238.6° C. |
| | DSC | | Endothermic event with onset at |
| | | | 173.4° C., peak 178.3° C. |
| DSC | 1st Heat | | Endothermic event with onset at |
| | | | 170.0° C., peak 179.3° C.; |
| | | | Additional endothermic event at 208.6 |
| | | | and 229.6° C. |
| NMR | Counterion | | N/A |
| | Residual Solvent | | Trace Ethyl Acetate |
| HPLC | Purity | % area | 97.87 |
| | CAD | % w/w | 12.4% |
| KF | Moisture | % w/w | 3.1 wt. % |
| | Content | | |
| DVS | Moisture Uptake at 80% RH | | 4.76 wt. % |
| | Moisture Uptake at 80% RH | | 5.18 wt. % |
| | XRPD | | Phosphate Pattern 7* |
| Slurry at | Ethanol/ | Aw 0.257 | Free Base Pattern 3 |
| 25° C. | water | Aw 0.507 | Free Base Pattern 3 |
| | | Aw 0.745 | Free Base Pattern 2 |
| | Water | XRPD | Free Base Pattern 10 |
| 7 Day | Ambient, | Observation | Faint Yellow Solid |
| Stability | Light | XRPD | Phosphate Pattern 7* |
| | | HPLC Purity, | 97.90 |
| | | % area | |
| | 40° C. / | Observation | Faint Pink Solid |
| | 75% RH | XRPD | Phosphate Pattern 7* |
| | | HPLC Purity, | 89.63 |
| | | % area | |
| | 80° C. | Observation | White Solid |
| | | XRPD | Phosphate Pattern 7* |
| | | HPLC Purity, | 98.08 |
| | | % area | |
| 24 h | pH 1.2 | Concentration, | 0.0057 |
| Solubility | | mL | |
| at 37° C. | | XRPD | HCl Pattern 1 |
| | pH 4.5 | Concentration, | 0.0052 |
| | | mg/mL | |
| | | XRPD | Free Base Pattern 1 |
| | pH 6.8 | Concentration, | 0.0042 |
| | | mg/mL | |
| | | XRPD | Free Base Pattern 1 |

### Example 40. Nonclinical Pharmacology

Secondary pharmacology studies evaluated the selectivity of Compound I, and safety pharmacology studies assessed the impact of Compound I on human ether-à-go-go-related gene (hERG)-mediated current, protein binding, and enzyme and uptake assays.

The potential for Compound I to inhibit hERG current at physiologic temperatures in stably transfected HEK293 cells was assessed. Experiments were performed at 37±1° C. Currents were measured using the whole-cell patch clamp method. Glass pipettes were pulled from borosilicate glass by a horizontal puller (Sutter Instruments, USA). Bath temperature was measured by a thermistor placed near the cell under study and were maintained by a thermoelectric device. An Axopatch 1-D amplifier of voltage clamp pulses and data acquisition was controlled by a computer running pClamp software (v10.7). The voltage protocol comprised: −80 mV for 100 ms, +40 mV for 500 ms, a −60 ms decreased slope of 1.2 V/see, then −80 mV for 3 s. Current was measured as outward current during ramp, with a pacing rate of 0.2 Hz (every 5 s), filtered at 2 kHz and digitized at 5 kHz. This protocol was repeated every 5 s until hERG tail current maintains stability, defined as approximately <10% change in current amplitude for at least 6 consecutive traces. Then the Compound I was applied as the protocol continues. Current was measured as the peak current during the ramp. The percent decrease in hERG current amplitude by Compound I was calculated from the peak current amplitudes after a steady-state level of drug block was achieved, relative to the current amplitude before drug was introduced (control). Values were derived using the average of 3 current traces.

Compound I inhibited hERG current amplitude with an IC₅₀ of 8.6 μM (FIG. 124). This concentration greatly exceeds the pharmacologically active concentrations required for inhibition of CDK2 and cellular proliferation.

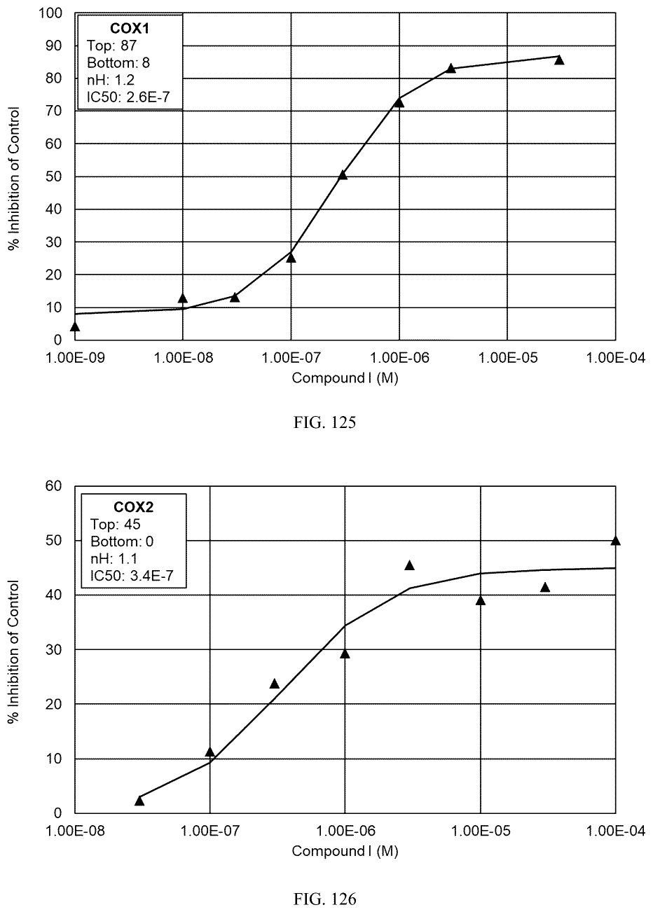

Compound I was tested at several concentrations for IC₅₀ determination. Human recombinant COX1 and COX2 were incubated with Compound I in the presence of arachidonic acid (3 μM) and ADHP (25 μM) and resorufin (oxidized ADHP) and were measured by fluorimetry (FIGS. 125-126). Receptor binding studies results suggest that Compound I is unlikely to cause adverse effects related to off target pharmacologic activity, as the lowest IC₅₀ value for off target activity was 260 nM for cyclooxygenase (COX) 1 (PTGS1) (FIG. 125), which is 464 times greater than the IC₅₀ value for inhibition of the intended target, CDK2/CCNE (0.56 nM).

TABLE 80
Calculated statistical parameters of Compound I inhibition assay.

| Transporter | IC₅₀ (μM) |
|---|---|
| COX1 | 260 |
| COX2 | 270 |

Safety pharmacology studies described more fully below included a functional observational battery (FOB) testing in a 28-day repeat-dose toxicity study in rats, electrocardiogram (ECG) evaluations in a 28-day repeat-dose toxicity study in dogs, and a single-dose respiratory safety pharmacology study in rats. Treatment with Compound I did not affect the FOB results, ECGs, or respiratory function at any dose level.

### Example 41. Nonclinical Pharmacokinetics

Nonclinical PK studies were conducted for single dose of Compound I in rats, mice, and dogs, and for repeated doses of Compound I in rats and dogs. Across all species tested, oral (PO) absorption of Compound I was rapid and half-life (t_{1/2}) was approximately 6 hours or less. The relationship between dose level and systemic exposure was generally similar in male and female animals.

In rats and dogs, a single dose of Compound I was generally quantifiable in the blood for up to 24 to 36 hours (after intravenous [IV] or PO dosing). Exposure to Compound I was approximately 1.3- to 2.4-fold higher in blood than plasma in mice (IV and PO dosing), approximately 2-fold higher in blood than plasma in rats (IV and PO dosing), and approximately 4-fold (IV dosing) to 6.5-fold (PO dosing) higher in blood than in plasma in dogs.

In all species (rats, mice, and dogs), the time to maximum concentration (Tₘₐₓ) occurred in 0.25 hours or less after IV dosing. After PO dosing, the Tₘₐₓ ranged from 0.25 to 0.5 hours in rats, 0.25 to 4.0 hours in mice, and 1.2 to 1.7 hours in dogs. Generally, the t_{1/2} ranged from approximately 2 to 4 hours in mice, 1 to 4 hours in rats, and 4.6 to 6.4 hours in dogs.

The preclinical development of Compound I included PK evaluations of different forms and formulations of the active pharmaceutical ingredient as well as measurement of in vivo concentrations in both plasma and whole blood. Initial dose formulation development led to the use of Compound I free base in polyethylene glycol (PEG)-400 (solution dosing with approximate solubility of 100 mg/mL) and Compound I HCl salt in methylcellulose (MC) or MC/Tween 80 (both MC formulations forming a suspension). An amorphous solid dispersion formulation of the compound has been developed (40% Compound I: hydroxypropyl methylcellulose acetate succinate [HPMCAS]-H spray dried dispersion [SDD]). In both rats and dogs, compared with the hydrochloric acid salt, the HPMCAS-H SDD formulation demonstrated improved exposures and plasma dose proportionality.

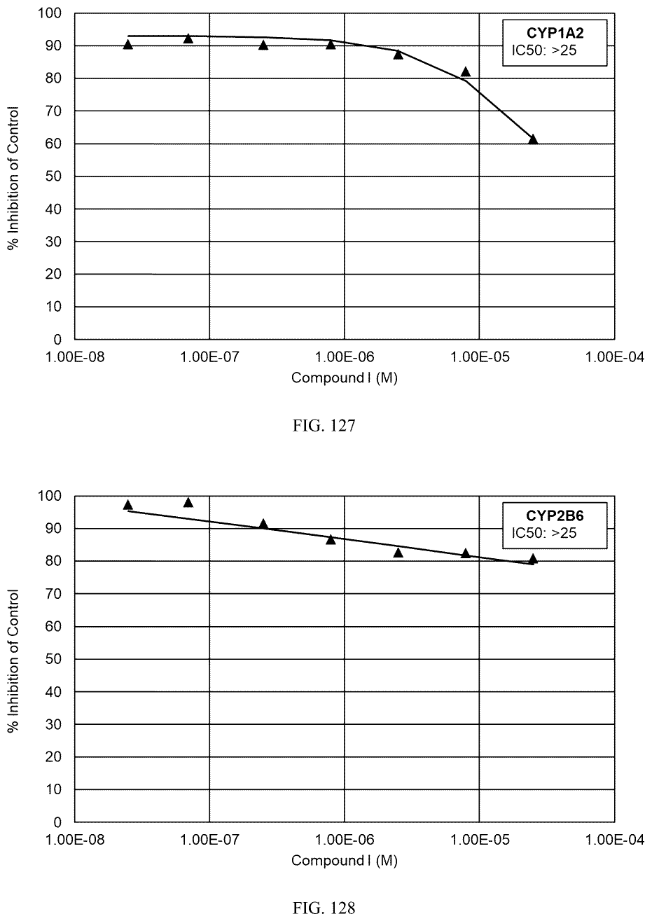

In vitro studies were performed to evaluate potential drug-drug-interaction risks, including CYP inhibition and the potential for Compound I to serve as an inhibitor of major human drug transporters (FIGS. 127-134). The potential clinical relevance of the in vitro DDI assessments will be evaluated when human PK data are available.

For inhibition assessments of the cytochrome P450 isoforms CYP1A2, CYP2B6, CYP2C8, CYP2C9, CY2C19, CY2D6, and CYP3A5, human liver microsomes were incubated in the appropriate isoform-specific probe substrate at 37° C. for a pre-determined incubation time in the presence of Compound I (0.025-25 μM prepared in acetonitrile: DMSO (9:1)) and NADPH (1 mM) in the presence of 100 mM potassium phosphate (pH 7.4) containing 5 mM magnesium chloride. The final DMSO content in the reaction mixtures was ≤0.2%. A selective inhibitor of each isoform was also screened as a positive control. Incubations were terminated by the addition of methanol. The quenched samples were incubated at 4° C. for 10 minutes and filtered by centrifugation for 1 minute at 500 rcf. The supernatants were removed, and the probe substrate metabolites were analyzed by LC-MS/MS.

The LC-MS/MS system consisted of an AB Sciex API5500 mass spectrometer coupled with an Agilent 1290 liquid chromatography system and an Agilent 1290 Infinity HTS chilled autosampler, controlled through Analyst software (AB Sciex). Samples were analyzed using the following solvent system and gradient: Acquity™ C18 Reverse Phase UPLC HSS T3 (1.8 μm) 2.1×50 mm column; 50° C. Column Temp.; 10 μL Injection Vol.; Mobile Phase A: Water+0.1% Formic Acid; Mobile Phase B: Acetonitrile+0.1% Formic Acid; Flow Rate: 0.6 mL/min. 0.05 min: 98% Mobile Phase A, 2% Mobile Phase B; 1.00 min: 5% Mobile Phase A, 95% Mobile Phase B; 1.80 min: 5% Mobile Phase A, 95% Mobile Phase B; 1.81 min: 95% Mobile Phase A, 2% Mobile Phase B; 2.80 min: 98% Mobile Phase A, 2% Mobile Phase B.

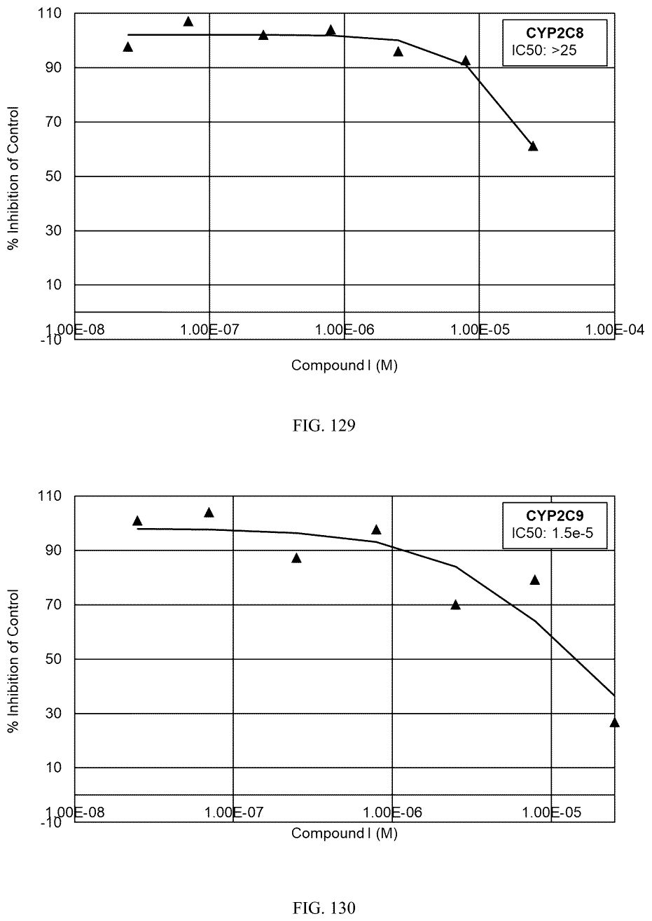

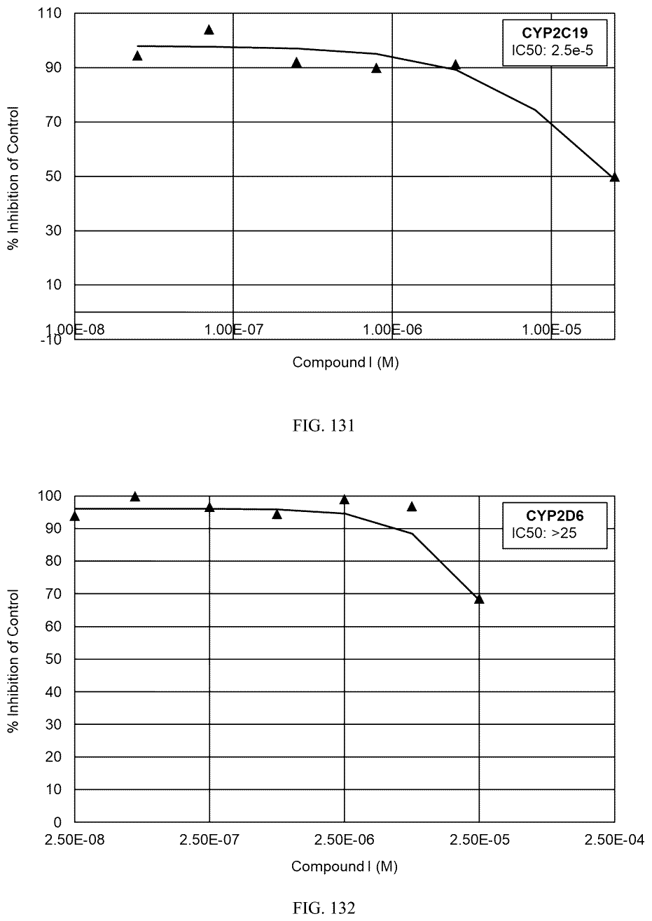

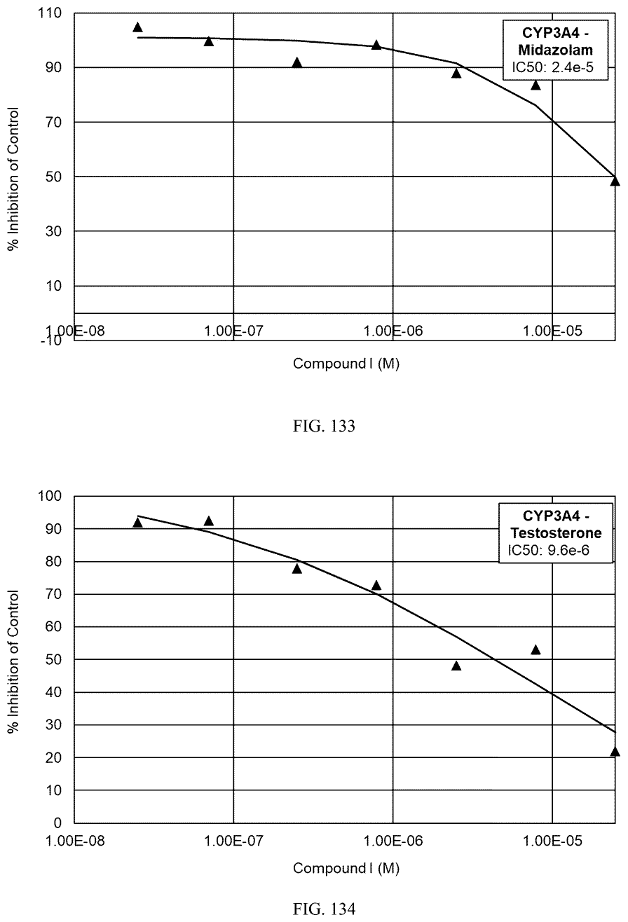

Compound I inhibited cytochrome P450 isoforms CYP2C9 (FIG. 130) and CYP3A4 (FIG. 133) (with testosterone as substrate) with weak to modest potency, with IC₅₀ values of 14.9 and 9.59 μM, respectively. Marginal to no inhibition was observed for CYP1A2 (FIG. 127), CYP2B6 (FIG. 128), CYP2C8 (FIG. 129), CYP2C19 (FIG. 131), CYP2D6 (FIG. 132), and CYP3A4 (midazolam as substrate) (FIG. 133) up to the highest concentration tested (25 μM). At concentrations up to 3 μM Compound I, there was no observed induction of CYP1A2, CYP2B6, or CYP3A4 transcription over 24 hours.

TABLE 81
Calculated statistical parameters of Compound I inhibition assay.

| Isoform | Substrate | IC₅₀ (μM) |
|---|---|---|
| CYP1A2 | Tacrine | >25 |
| CYP2B6 | Bupropion | >25 |
| CYP2C8 | Amodiaquine | >25 |
| CYP2C9 | Tolbutamide | 14.9 |
| CYP2C19 | Mephenytoin | 25.0 |
| CYP2D6 | Dextromethorphan | >25 |
| CYP3A4 | Midazolam | 24.3 |
| CYP3A4 | Testosterone | 9.59 |

To determine whether Compound I is an inhibitor of ATP-binding cassette (ABC) and/or solute carrier (SLC) transporters, were prepared from cells overexpressing human ABC (efflux) or SLC transporters, respectively.

For inhibition assessments of the ABC transporter BCRP, inside-out membrane vesicles were incubated in the respective assay buffer containing the probe substrate in the presence of Compound I (0.04-30 μM). The reaction was initiated with the addition of ATP or AMP to the solution and following the incubation time, the reaction was stopped by the addition of 200 μL of ice-cold washing buffer and immediate filtration via glass fiber filters mounted to a 96-well plate (filter plate). The filters were washed, dried, and the amount of substrate inside the filtered vesicles was determined by liquid scintillation counting. E3S (1 μM; Sigma-Aldrich E0251) for BCRP was used as positive control substrates.

For inhibition assessments of the SLC transporters, HEK293 cells expressing MATE1, MATE2-K, OAT1, OAT3, OATP1B1, OATPIB3, OCT1, or OCT2 were pre-incubated for 30 minutes in the respective assay buffer in the presence of Compound I (0.31-20 μM). Then the probe substrate was added in a fresh dosing solution of Compound I and following the incubation time, the reaction was stopped by washing the cells with ice-cold buffer. The cells were lysed with 0.1 M NaOH solution and the accumulation for the probe substrate in the cells was determined by liquid scintillation counting. Metformin (10 μM; Moravek Biochemicals Brea MC2043) for MATE1, MATE2-K, OCT, Sumatriptan (10 μM; Sigma-Aldrich S1198) for OCT1, Tenofovir (5 μM; Sequoia Research Products Ltd SRP01194t) for OAT1, E3S (1 μM; Sigma-Aldrich E0251) for OAT3, E217BG (1 μM; Sigma-Aldrich E-1127) for OATP1B1 and CCK-8 (1 μM; Sigma-Aldrich C2901) for OATP1B3 were used as positive control substrates.

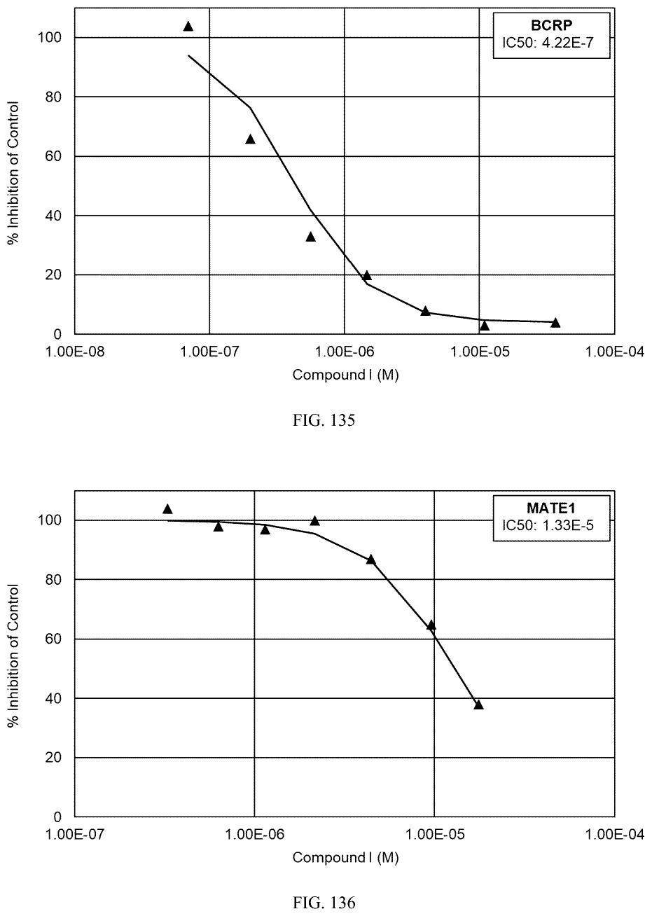

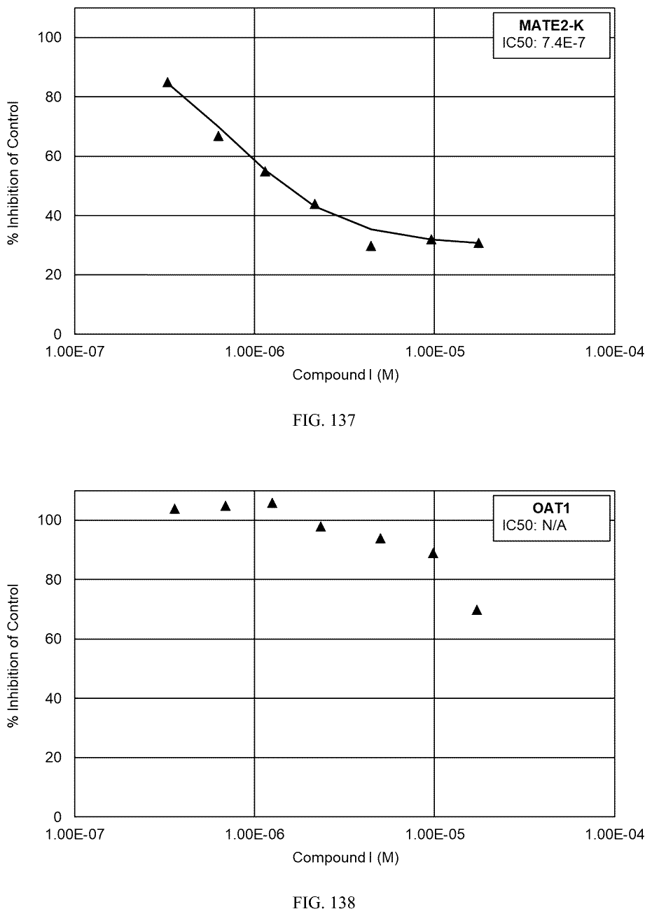

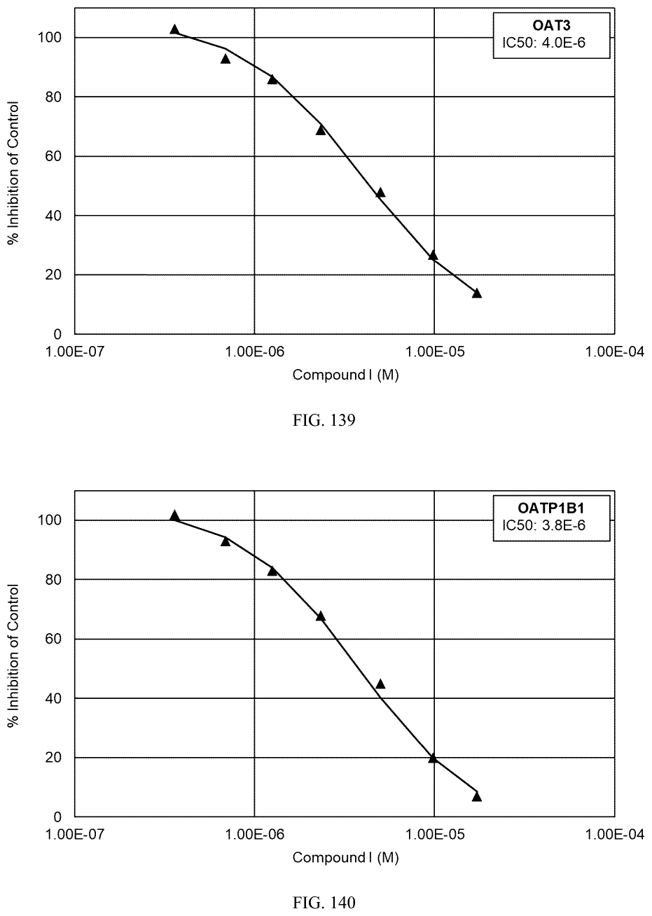

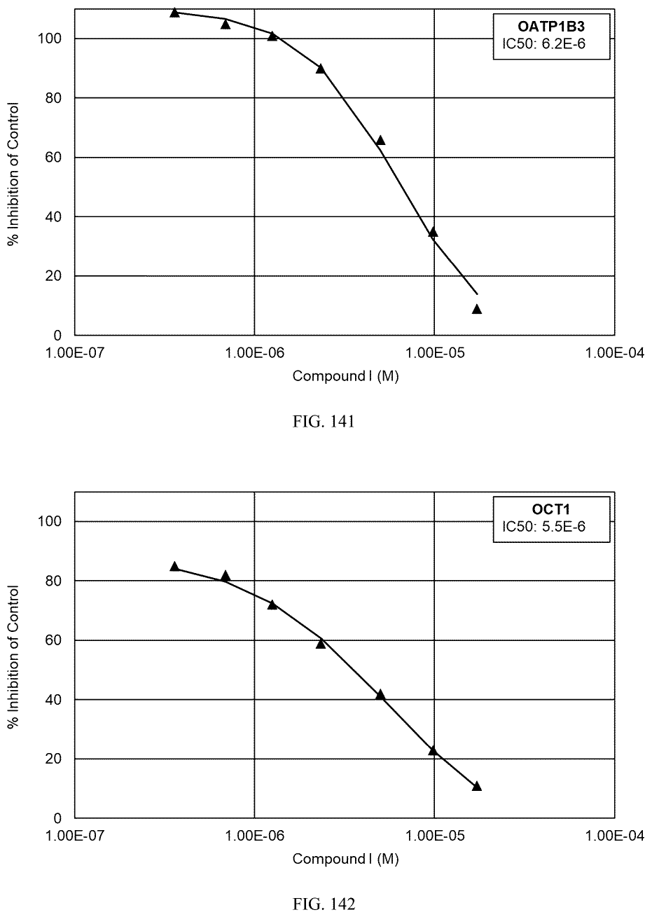

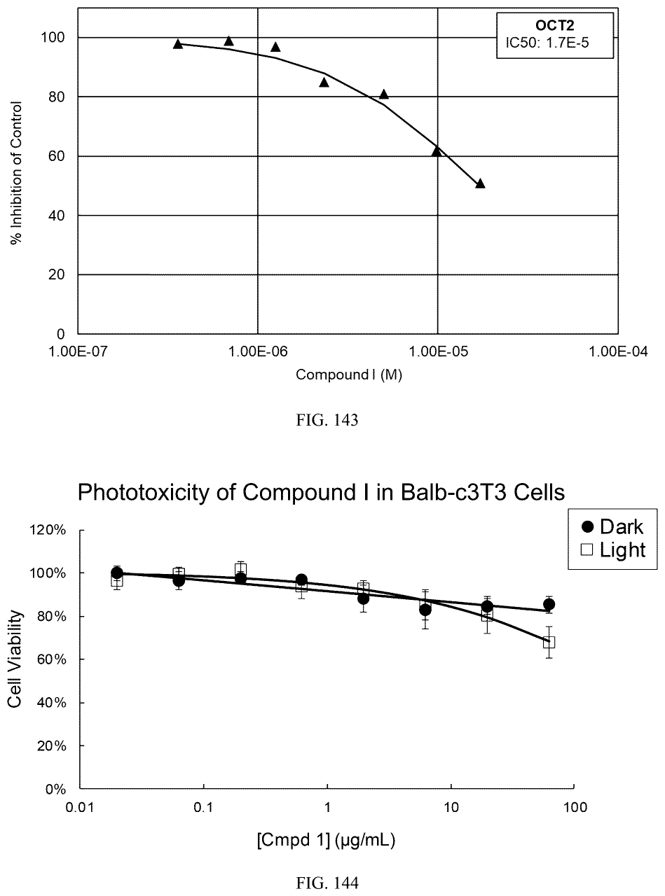

The in vitro interaction (inhibition) potential of Compound I with the human BCRP, MATE1, MATE2-K, OAT1, OAT3, OATP1B1, OATP1B3, OCT1, and OCT2 transporters was tested at 7 concentrations of Compound I (FIGS. 135-143). Compound I inhibited the MATE1-mediated metformin accumulation in a dose-dependent manner with a maximum inhibition of 62% observed at 20 μM nominal and 17.62 μM actual concentration (FIG. 136). The calculated IC₅₀ value was 13.29 μM (Table 82). Compound I was a potent inhibitor of breast cancer resistance protein (BCRP) and multidrug and toxic compound extrusion (MATE) 2-K, with IC₅₀ values of 0.422 and 0.740 M, respectively (FIGS. 135, 137). The compound inhibited other transporters with lower potency, including organic anion transporting polypeptide (OATP) 1B1 (FIG. 140), organic anion transporter (OAT) 3 (FIG. 139), organic cation transporter (OCT) 1 (FIG. 142), and OATP1B3 (FIG. 141) with IC₅₀ values of 3.81, 4.01, 5.50, and 6.22 μM respectively, and with still lower potency for MATE1 (FIG. 136) and OCT2 (FIG. 143), with IC₅₀ values of 13.3 and 17.1 μM, respectively. Compound I did not inhibit bile salt export pump (BSEP), multidrug resistance mutation (MDR) 1, or OAT1 (FIG. 138).

TABLE 82
Calculated statistical parameters of transporter
Compound I inhibition assay.

| | Maximum Percent | |
|---|---|---|
| Transporter | inhibition | IC₅₀ (μM) |
| BCRP | | 0.4223 |
| MATE1 | 62 | 13.29 |
| MATE2-K | 69 | 0.7402 |
| OAT1 | 30 | N/A |
| OAT3 | 86 | 4.008 |
| OATP1B1 | 93 | 3.810 |
| OATP1B3 | 91 | 6.223 |
| OCT1 | 89 | 5.504 |
| OCT2 | 49 | 17.06* |

*estimated.

Compound I had moderate permeability, with A-B Papp ranging from 3.28×10⁻⁶ to 6.67×10−6 cm/s and B-A Pₐₚₚ ranging from 42.0×10⁻⁶ to 68.3×10−6 cm/s. Compound I appears to undergo active efflux that is likely mediated by BCRP and MDR1, with net efflux ratios of up to 56.65 and 32.28, respectively. Across all species tested, >94.4% of Compound I was bound to plasma proteins.

### Example 42. Nonclinical Toxicology

The purpose of this study was to characterize the toxicity and toxicokinetics of Compound I when administered once daily for 28 consecutive days via oral gavage to male and female Sprague Dawley rats. One-hundred and sixty (80 per sex) Sprague Dawley rats were randomly assigned to groups and dosed once daily via oral gavage for 28 consecutive days.

Toxicokinetic analyses were performed on composite plasma concentration versus time data for Compound I using Phoenix WinNonlin (version 8.1) non-compartment analysis function (linear trapezoidal rule for AUC calculations) sparse data analysis function. Nominal dose values and sampling times were used for calculations. Any concentration reported as BLQ (LLOQ<1.0 ng/ml) was set equal to zero. Toxicokinetic analyses included Cₘₐₓ, Tₘₐₓ, AUC₀₋₂₄, t_{1/2}, RA Cₘₐₓ, and RA AUC₀₋₂₄.

Daily or twice-daily PO administration of Compound I was generally well tolerated for up to 28 days at doses up to 1000 mg/kg/day in the rat and doses up to 250 mg/kg BID in the dog.

In the Good Laboratory Practice (GLP)-compliant 28-day toxicity study, daily PO doses of Compound I up to 1000 mg/kg/day were well tolerated in rats without adverse effects of any sort. Treatment with Compound I did not affect mean beady weight nor mean body weight gain at any dose level. Based on the absence of toxicity, the 28-day maximum tolerated dose (MTD) (also the no observed adverse effect level [NOAEL]) was 1000 mg/kg/day, the highest dose level tested. After the last dose at the 28-day MTD, plasma maximum concentration (Cₘₐₓ) and area under the concentration-time curve (AUC)₀₋₂₄ were 647 ng/ml and 8130 h*ng/ml, respectively, for males and 1390 ng/mL and 12700 h*ng/mL, respectively, for females (Table 83). There was no accumulation of Compound I after 28 days of once-daily dosing. Functional Observational Battery (FOB) and Grip Strength was not affected by Compound I at any dose level. Respiratory function was also not affected by Compound I at any dose level.

TABLE 83
Compound I toxicokinetics in 28-day rat study.

| | Dose | | | Cₘₐₓ | SECₘₐₓ | AUC₀₋₂₄ | SE | | | |
|---|---|---|---|---|---|---|---|---|---|---|
| | (mg/ | | Tₘₐₓ | (ng/ | (ng/ | (hr * ng/ | AUC₀₋₂₄ | t_{1/2} | RA | RA |
| Day | kg/day) | Sex | (hr) | mL) | mL) | mL) | (hr * ng/mL) | (hr) | Cₘₐₓ | AUC₀₋₂₄ |
| 1 | 100 | F | 0.25 | 2450 | 408 | 7570 | 601 | 3.78 | — | — |
| | | M | 0.25 | 1610 | 215 | 8310 | 1230 | 3.27 | — | — |
| | 300 | F | 0.50 | 4290 | 483 | 11600 | 1170 | 5.28 | — | — |
| | | M | 0.50 | 2770 | 422 | 10700 | 1150 | 5.54 | — | — |
| | 1000 | F | 0.50 | 3590 | 580 | 12600 | 1290 | 5.89 | — | — |
| | | M | 0.50 | 1370 | 262 | 11300 | 740 | 9.35 | — | — |
| 28 | 100 | F | 0.25 | 3330 | 903 | 9580 | 490 | 3.93 | 1.36 | 1.27 |
| | | M | 8.0 | 1010 | 137 | 11000 | 820 | NA | 0.627 | 1.32 |
| | 300 | F | 0.50 | 3070 | 895 | 11400 | 567 | 3.98 | | 0.982 |
| | | M | 1.0 | 887 | 247 | 9750 | 1570 | 5.34 | | 0.908 |
| | 1000 | F | 0.25 | 1390 | 100 | 12700 | 1460 | 9.48 | | 1.01 |
| | | M | 8.0 | 647 | 34.5 | 8130 | 1210 | NA | | 0.717 |

Note:
F = female; M = male.

Toxicokinetic analyses were performed on composite plasma concentration versus time data for Compound I using Phoenix WinNonlin (version 8.1) non-compartment analysis function (linear trapezoidal rule for AUC calculations) sparse data analysis function. Nominal dose values and sampling times were used for calculations. Any concentration reported as BLQ (LLOQ<1.0 ng/mL) was set equal to zero. Toxicokinetic analyses included Cₘₐₓ, Tₘₐₓ, AUC₀₋₂₄, t_{1/2}, RA Cₘₐₓ, and RA AUC₀₋₂₄. Forty (20 per sex) Beagle dogs were randomly assigned to groups and dosed twice daily (approximately 8 hours+1-hour apart) via oral gavage for 28 consecutive days.

In the GLP-compliant 28-day dog toxicity study, PO doses of Compound I at up to 250 mg/kg BID were well tolerated with no dose-limiting toxicities observed. Based on the absence of dose-limiting toxicity, the 28-day highest nonseverely toxic dose (HNSTD) was 250 mg/kg BID (500 mg/kg/day). On the last day of dosing at the 28-day HNSTD, plasma Cₘₐₓ and AUC₀₋₂₄ were 1500 ng/ml and 18800 h*ng/mL, respectively, for males and 961 ng/mL and 11700 h*ng/mL, respectively, for females (Table 84). Based on the occurrence of moderately decreased bone marrow cellularity at the low-dose level, the study did not identify a 28-day NOAEL. On the last day of dosing at the low-dose level, plasma Cₘₐₓ and AUC₀₋₂₄ were 307 ng/ml and 2460 h*ng/mL, respectively, for males and 267 ng/ml and 2600 h*ng/ml, respectively, for females. Modest accumulation was observed at all dose levels after 28 days of BID dosing. All electrocardiograms were qualitatively and quantitatively within normal limits for dogs at all dose levels of Compound I.

TABLE 84
Compound I toxicokinetics in 28-day dog study.

| | Dose | | | | AUC₀₋₂₄ | | |
|---|---|---|---|---|---|---|---|
| | (mg/kg/ | | Tₘₐₓ | Cₘₐₓ | (hr * ng/ | RA | RA |
| Day | day) | Sex | (hr) | (ng/mL) | mL) | Cₘₐₓ | AUC₀₋₂₄ |
| 1 | 20 | F | 9.2 | 166 | 1680 | — | — |
| 1 | 20 | M | 11 | 137 | 1430 | — | — |
| 1 | 100 | F | 12 | 497 | 5170 | — | — |
| 1 | 100 | M | 12 | 459 | 6210 | — | — |
| 1 | 500 | F | 14 | 919 | 10100 | — | — |
| 1 | 500 | M | 14 | 1220 | 13300 | — | — |
| 28 | 20 | F | 8.8 | 267 | 2600 | 1.65 | 1.70 |
| 28 | 20 | M | 4.5 | 307 | 2460 | 2.37 | 1.75 |
| 28 | 100 | F | 10 | 725 | 9160 | 1.62 | 2.00 |
| 28 | 100 | M | 16 | 841 | 10600 | 1.95 | 1.84 |
| 28 | 500 | F | 7.4 | 961 | 11700 | 1.69 | 1.89 |
| 28 | 500 | M | 9.2 | 1500 | 18800 | 1.65 | 1.74 |

Note:
F = female; M = male.

Compound I was screened for its potential to cause phototoxicity in the in vitro 3T3 NRU phototoxicity test. BALB/c-3T3 mouse fibroblasts (ATCC) were seeded in 96-well plates at 15,000 cells per well, and grown for 24 hr in DMEM supplemented with 10% FBS, 100 U/mL penicillin, and 100 mg/L streptomycin at 37° C., humidified with 5% CO2.

All test articles were solubilized and serially diluted in DMSO, with the exception of histidine, which was solubilized and diluted in HBSS. All DMSO stocks were diluted in HBSS at the final testing concentrations. The final concentration of DMSO for all test articles was 1%. At the start of the assay, the growth medium was removed from the plates and replaced with test article dilutions. Six replicates were used for each concentration and two plates were prepared for each test article. The cells were incubated in the dark at 37° C., humidified with 5% CO2 with test articles for 1 hr. After the 1 hr incubation period, one plate of each test article was exposed to UVA light, 2.5 J/cm² over 18 min which is sufficient to induce a phototoxic response, while the second plate was incubated in the dark at room temperature for the same period of time. At the end of this incubation period, both plates were washed with PBS and fresh medium was added to each well. The plates were incubated at 37° C., humidified with 5% CO2 overnight. After the overnight incubation, medium was removed and fresh medium containing 33 μg/mL of Neutral Red (from Sigma) was added. After a 3-hour incubation with Neutral Red, the cells were washed with PBS, and the cellular dye was solubilized with 1% acetic acid in 50% ethanol. Cellular Neutral Red was measured by its absorbance at 540 nm. Compound I was found to be negative for causing phototoxicity (FIG. 144).

### Example 43. Compound I Prolongs the Time to CDK4/6 Inhibitor Resistance

While approximately 10-15% of patients with ER+ breast cancer present with de novo resistance to CDK4/6 inhibitor therapy, nearly all patients ultimately develop acquired resistance. Given that ER+ breast cancers represent a substantial subpopulation of CDK4/6 inhibitor resistant subjects, Compound I was tested alone or in combination with CDK4/6 inhibitors in ER+ breast cancer models.

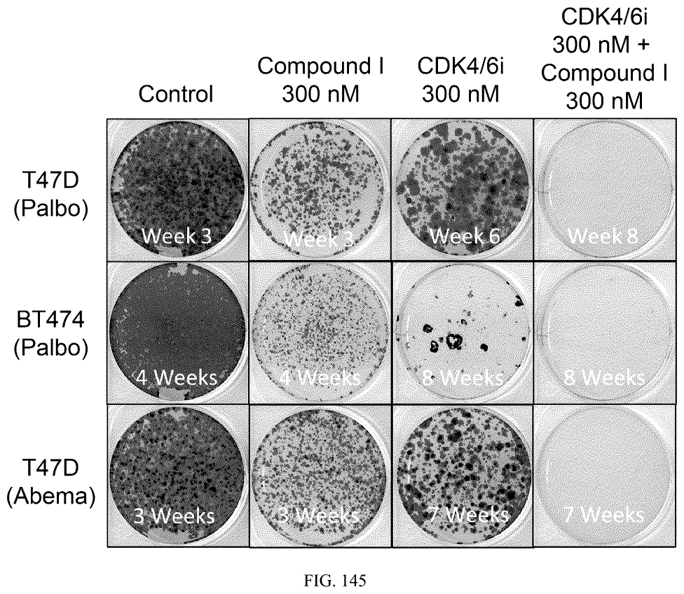

The T47D human breast carcinoma cell line was grown in RPMI-1640/1× Glutamax/1× Insulin/10% fetal bovine serum and was plated at 10,000 cells/well in Corning 6 well tissue culture treated plates. The BT474 human breast carcinoma cell line was grown in DMEM/1× Glutamax/1× Insulin/10% fetal bovine serum and was plated at 25,000 cells/well in Corning 6 well tissue culture treated plates. Cells were incubated at 37° C. for 24 hours prior to addition of treatment. 4 plates were treated with 0.1% DMSO and 300 nM CDK4/6i (either palbociclib or abemaciclib as indicated) in triplicate, 2 plates were treated with combination of 300 nM Compound I and 300 nM CDK4/6i, or 300 nM Compound I, and 2 plates were treated with combination of 300 nM Compound I and 300 nM CDK4/6i, or 300 nM CDK4/6i. Plates were collected at week 2 and 3, at week 3, 2 plates with only CDK4/6i were switched to combination treatment. The remaining plates were collected at weeks 5 and 8. Collection was performed by washing wells with PBS before incubating 5 minutes with 0.1% crystal violet/20% methanol. Dye was removed and wells washed 4 times with PBS before being scanned at 1200 dpi. Image J was used to count the % area and colony size for each well. Graph pad was used for analysis. Compound I prolonged the time to CDK4/6i resistance in both the T47D and BT474 ER+ breast cancer model cells (FIG. 145). The treatment of cells with both Compound I and a CDK4/6i resulted in no noticeable growth after 8 weeks.

### Example 44. Compound I Results in Tumor Regression and Stasis in Ovarian and Gastric CCNE1-Amplified Tumor Models

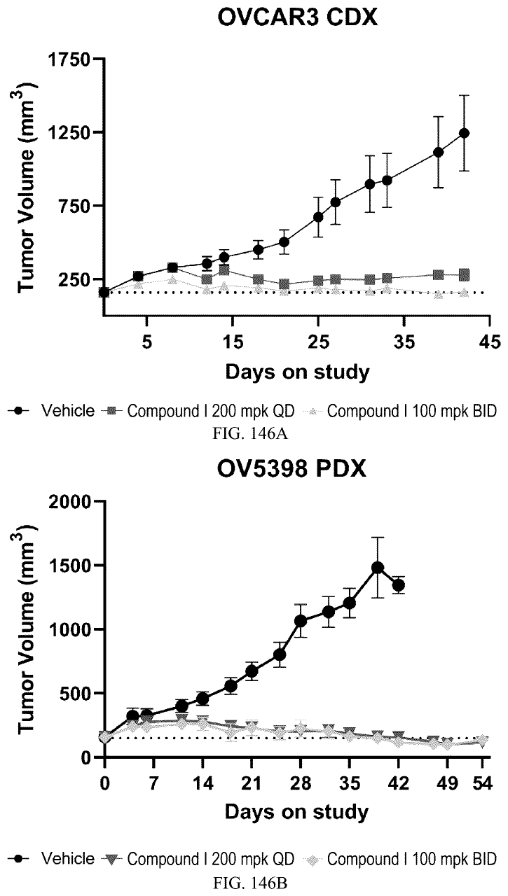

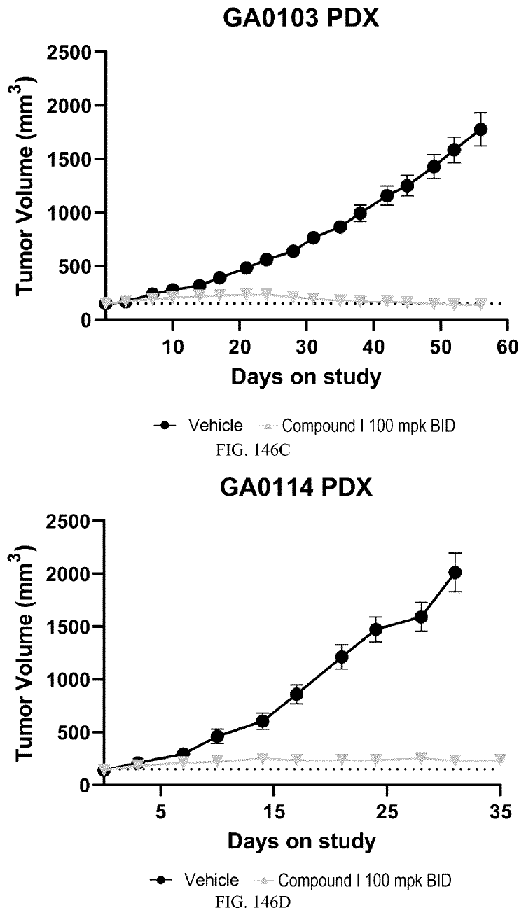

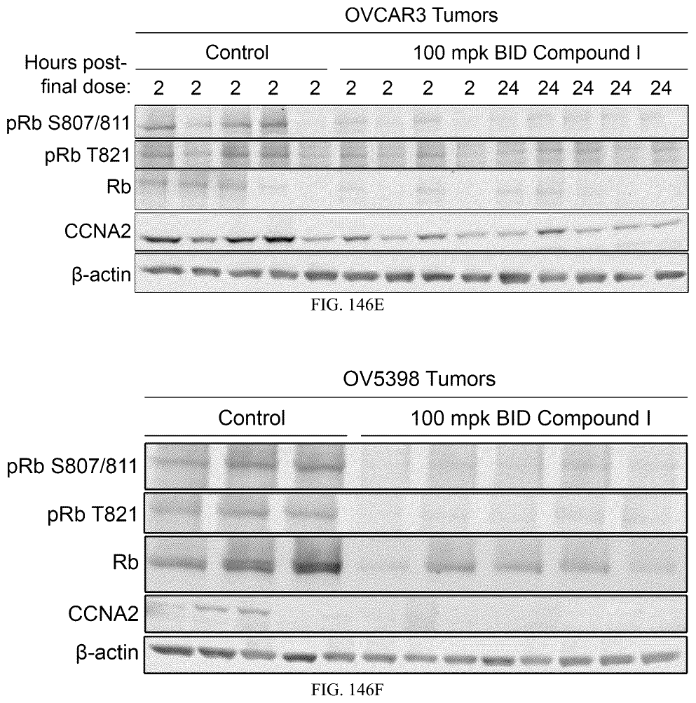

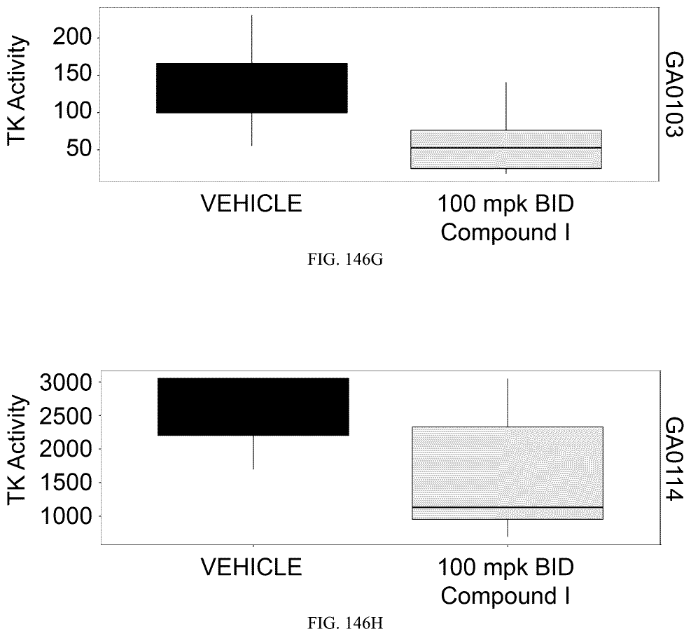

Compound I was administered in different dosing schemes to ovarian and gastric CCNE1-amplified tumor models to assess tumor inhibition in vivo (FIG. 146A-146D).

10 million OVCAR3 human ovary carcinoma cells were injected into athymic nude female mice. The study was initiated at a mean tumor volume of approximately 100-200 mm³. Compound I was dosed at 100 mpk BID, as well as 200 mpk QD. The test agents were delivered at 5 mL/kg by oral gavage. Surprisingly, results show Compound I-mediated durable tumor stasis or regression out to 42 days post-dose initiation (FIG. 146A).

A 2-3 mm³ piece of OV5398 human ovary patient derived xenograft (PDX) tumor was injected into NOD/SCID female mice. The study was initiated at a mean tumor volume of approximately 150-250 mm³. Compound I was dosed at 100 mpk BID, as well as 200 mpk QD. The test agents were delivered at 5 mL/kg by oral gavage. Unexpectedly, results show Compound I-mediated tumor regression out to 56 days post dose initiation (FIG. 146B).

A 2-3 mm³ piece of GA0103 human gastric patient derived xenograft (PDX) tumor was injected into BALB/c nude female mice. The study was initiated at a mean tumor volume of approximately 150 mm³. Compound I was dosed at 100 mpk BID. The test agents were delivered at 5 mL/kg by oral gavage. These results show Compound I-mediated tumor regression out to 56 days post dose initiation (FIG. 146C).

A 2-3 mm³ piece of GA0114 human gastric patient derived xenograft (PDX) tumor was injected into BALB/c nude female mice. The study was initiated at a mean tumor volume of approximately 140 mm³. Compound I was dosed at 100 mpk BID. The test agents were delivered at 5 mL/kg by oral gavage. These results show Compound I-mediated tumor growth inhibition of 95% out to day 35 post dose initiation (FIG. 146D).

Tumors from FIG. 146A were harvested at the end of study and snap frozen. A small piece of each tumor was removed while frozen and lysed with 150 μL cold RIPA with protease and phosphatase inhibitors while being homogenized with mortar and pestle. Lysates were clarified by centrifugation at 14 kxg for 10 min at 4° C. and supernatants transferred to clean tubes. A BCA assay was performed to determine the protein concentration of each lysate. 30 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBST, membranes were incubated for 45 minutes in LI-COR secondary antibodies. Following 2 washes in TBST, and one TBS wash, membranes were scanned using a LI-COR OdysseyCLx. Image studio was used for image analysis. Compound I administered to OVCAR3 mice at 100 mpk BID sustainably substantially reduces Rb1 phosphorylation and cyclin A2 levels in ex vivo tumor samples at least 24 hours post-final dose (FIG. 146E).

Tumors from FIG. 146B were harvested at the end of study and snap frozen. A small piece of each tumor was removed while frozen and lysed with 150 μL cold RIPA with protease and phosphatase inhibitors while being homogenized with mortar and pestle. Lysates were clarified by centrifugation at 14 kxg for 10 min at 4° C. and supernatants transferred to clean tubes. A BCA assay was performed to determine the protein concentration of each lysate. 30 μg of protein was mixed with sample buffer and reducing agent before being run on a 4-12% SDS-PAGE. Protein was transferred to a nitrocellulose membrane and blocked with LI-COR blocking buffer for 1 hour. Membranes were incubated overnight in primary antibody at 4° C. Following 3 washes with TBST, membranes were incubated for 45 minutes in LI-COR secondary antibodies. Following 2 washes in TBST, and one TBS wash, membranes were scanned using a LI-COR OdysseyCLx. Image studio was used for image analysis. Compound I administered to OV5398 mice at 100 mpk BID sustainably substantially reduces Rb1 phosphorylation and cyclin A2 levels in ex vivo tumor samples at least 24 hours post-final dose (FIG. 146F).

Serum thymidine kinase levels were then analyzed to evaluate the CDK4/6 pathway functionality in these CCNE1-amplified tumor models. Thymidine kinase (TK), downstream of the CDK 4/6 pathway, is a marker of active cell division. Plasma from GA0103 and GA0114 PDX models were collected 2 to 24 hours post-final dose and frozen for later analysis. Analysis of thymidine kinase (TK1) activity in the plasma was analyzed using an ELISA produced by Biovica. Samples showed that mice treated with vehicle control had higher levels of TK1 activity in the plasma compared to mice treated with 100 mpk BID Compound I, indicating tumor cells were arrested by Compound I in two independent PDX models (FIG. 146G-146H).

### Example 45. Compound I Restores Sensitivity in Cells Resistant to Previous CDK4/6 Inhibitor and/or Anti-Estrogen Therapy

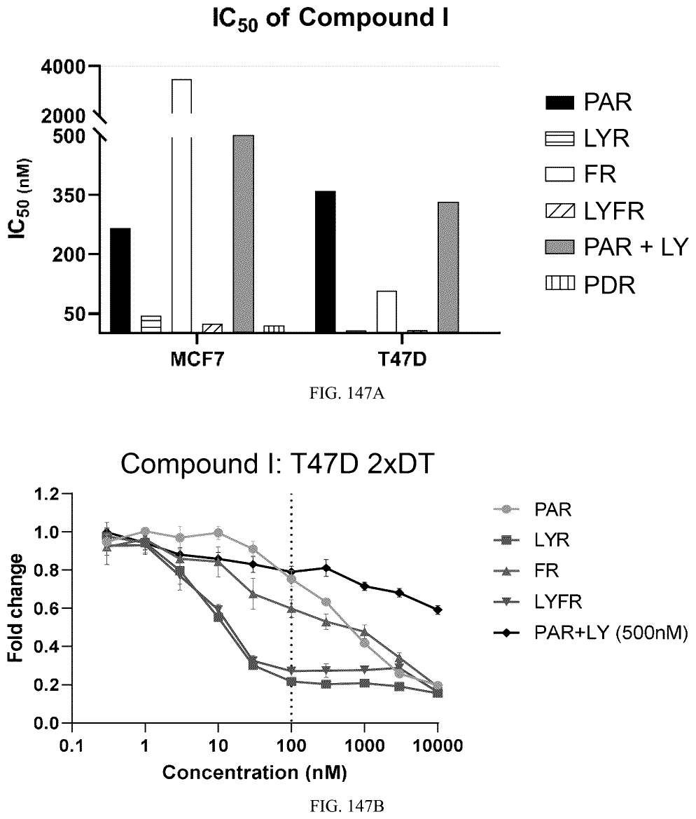

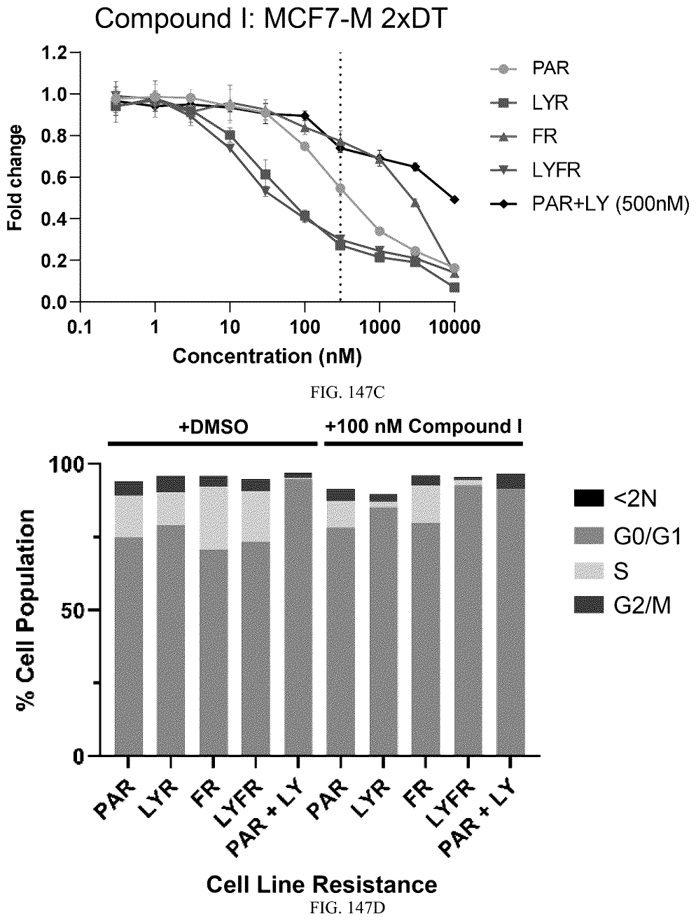

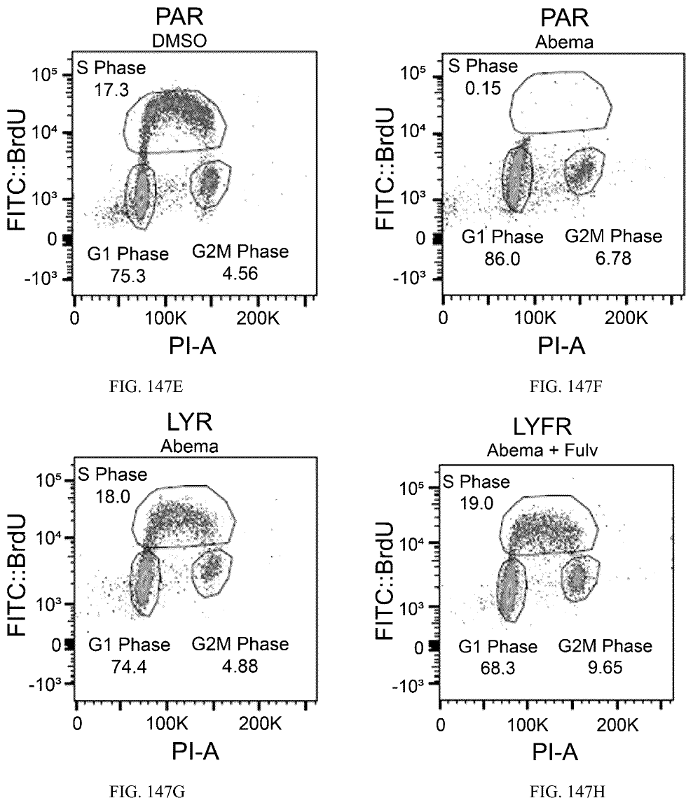

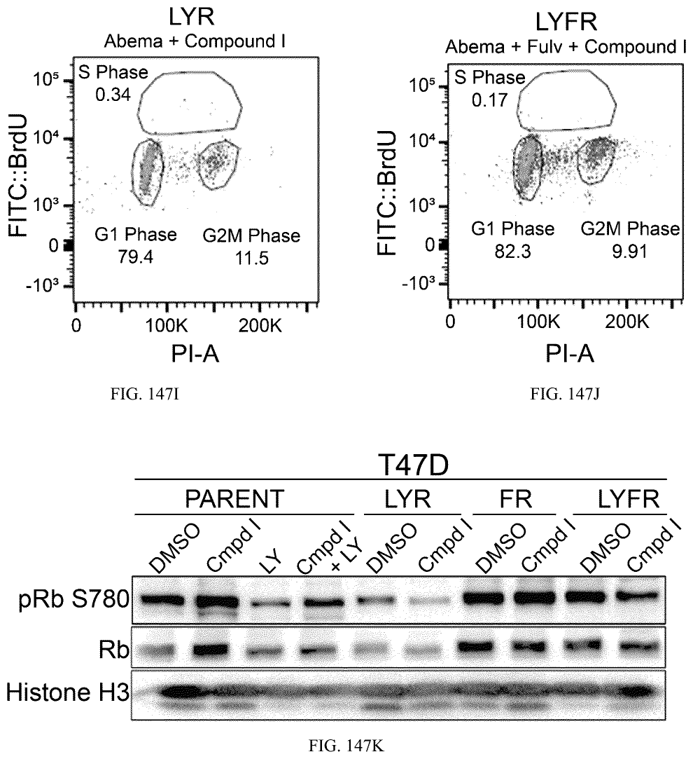

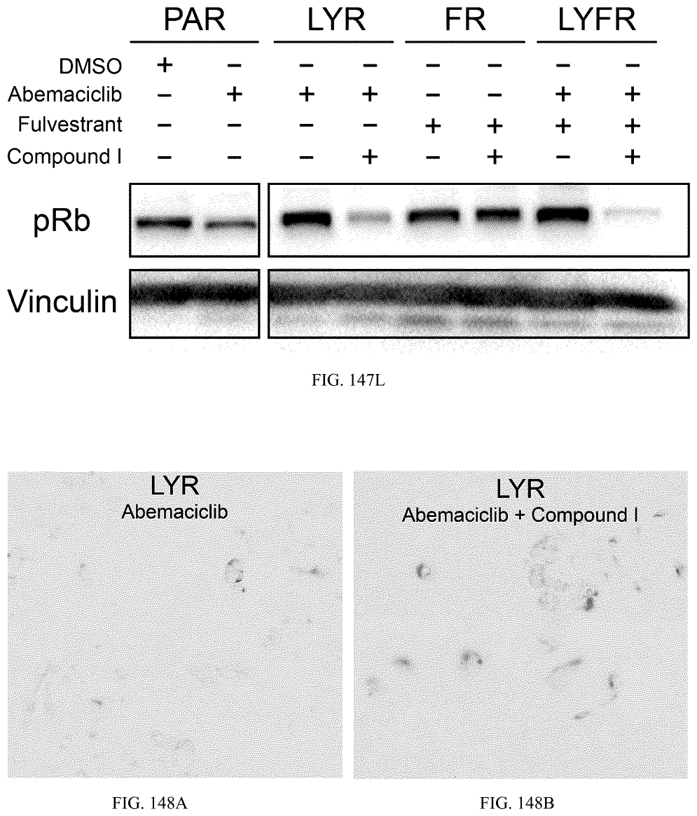

Parental (PAR) MCF7 and T47D luminal breast cancer cells and therapy-resistant daughter cells (LYR, FR, LYFR, PAR+LY, PDR) were treated with Compound I and the corresponding resistant drug fixed as multiple-point dose response to analyze the anti-proliferative activity of Compound I in therapy-resistant cells. Cells were incubated at 37° C. for 24 hours prior to addition Compound I and the corresponding resistant drug. The doubling time of each cell line was calculated and plates were removed from incubator after 2 doubling times. Cells were fixed and stained with DAPI and imaged with automated counting of cell number. Compound I exhibited low, nanomolar IC₅₀ values in cells with resistance to CDK4/6i (FIG. 147A). Compound I regulated a dose-dependent decrease in cellular proliferation in abemaciclib-resistant and/or fulvestrant resistant cells (FIG. 147B-147C). These results indicate that Compound I restores sensitivity to CDK4/6 inhibition in cells resistant to CDK4/6 inhibition and/or anti-estrogen therapy showing potent anti-proliferative activity.

The parental (PAR) T47D luminal breast cancer cells and therapy-resistant daughter cells (LYR, FR, LYFR, PAR+LY, PDR) were used to analyze cell-cycle status in response to Compound I as compared to DMSO control. The therapy resistant cells were previously incubated with abemaciclib, fulvestrant, and/or palbociclib for prolonged periods to the point of developing drug resistance. Cells were plated in Corning 6 well tissue culture treated plates. Cells were treated for 7 days. Media was spiked with 10 μM BrdU prior to collection. Cells were washed in PBS, trypsinized, collected and centrifuged at 300 RCF for 5 mins. Cells were resuspended in cell culture media and transferred into a 96 well V-bottom plate with roughly 300,000 cells per well. Cells were then stained using the Near-IR Fixable Viability Stain (Invitrogen Cat. L34976) for 20 mins at room temperature, protected from light. Cells were fixed and permeabilized using eBioscience FoxP3/Transcription Factor Staining Buffer Set (Cat. 00-5523-00). DNA was denatured using 2N HCl+0.5% (v/v) Triton X-100 for 30 mins room temperature, agitating every 5-10 mins. The acid was neutralized using 0.1M Na2B4O7·10H2O (pH 8.5) followed by 0.5% BSA in PBS. Cells were then stained with BrdU antibody (Clone 3D4, BioLegend Cat. 364118) for 1 hour at room temperature. Prior to acquisition on the BD A3 Symphony, DNA was stained for 30 minutes using FxCycle Violet ReadyFlow Reagent. A minimum of 10,000 live cell events were recorded for each sample Therapy-resistant T47D cells were arrested in G1 when administered Compound I in addition to abemaciclib (FIG. 147D), exhibiting a senescent-like phenotype.

Cell staining was then conducted to monitor cellular proliferation and perform cell cycle analysis of these therapy-resistant cells. While abemaciclib arrested parental (PAR) cell lines in the G1 phase (FIG. 147F), abemaciclib-resistant cells (LYR) administered abemaciclib were observed substantially less frequently in the G1 phase (FIG. 147G). Similarly, abemaciclib- and fulvestrant-resistant (LYFR) cells administered abemaciclib and fulvestrant were not substantially arrested in the G1 phase (FIG. 147H). In sharp contrast, Compound I arrested therapy-resistant LYR cells (FIG. 147I) and LYFR cells (FIG. 147J) when administered in combination with abemaciclib or abemaciclib and fulvestrant, respectively. These results demonstrate that Compound I re-sensitizes therapy-resistant cells to cell cycle arrest when administered with the therapies to which the cells developed resistance to.

Retinoblastoma (Rb) phosphorylation status analysis was then conducted in therapy-resistant cells by Western blotting. The T47D human breast carcinoma cell line was grown in RPMI-1640/1× Glutamax/1× Insulin/10% fetal bovine serum and was plated in Corning 6 well tissue culture treated plates. Cells were incubated at 37° C. for 24 hours prior to addition of treatment. Treatments were either DMSO, Compound I, or abemaciclib alone, or Compound I in combination with abemaciclib. Cells were treated for 7 days. All Abemaciclib treated cells received 500 nM Abemaciclib. MCF7M cells received 300 nM Compound I, T47D cells received 100 nM Compound I. For samples normalized to protein concentration (cPARP, BCL-xL and BCL-2), cell samples were collected by collecting media, PBS wash and trypsinized cells, centrifuging at 500 xg for 4 min at 4° C. Supernatant was removed, pellet resuspended in 250 μL of 1× Cell Lysis buffer (CST #9803, 1 tablet of PhosSTOP, 1 tablet of complete Protease Inhibitor Cocktail in 5 mL) then incubated on ice for 20 mins. Lysate was centrifuged at 21,000×g for 20 min at 4° C. and supernatant transferred to clean tubes. A DC assay was performed to determine the protein concentration of each lysate. 20 μg of protein was mixed with Laemelli buffer (+5% BME) before being run on homemade 15% gels. For samples normalized to cell number (p-RB and total RB), cells were washed in ice-cold PBS. Cells were counted and 500,000 cells were aliquoted into clean tubes. Cells were centrifuged at 500×g for 4 mins at 4° C. and supernatant discarded. Cell pellets were directly lysed in 100 μL of Laemmli buffer (+5% BME) and boiled at 95° C. for 12 min. 40,000 cells (8 μL) were run on homemade 10% gels. Protein was transferred to a nitrocellulose membrane and blocked with 5% milk for 1 hour. Membranes were incubated overnight in primary antibody at 4° C. Following 3 washes with TBST, membranes were incubated for 1 hour in secondary antibodies conjugated to HRP enzyme. Following 3 washes in TBST, membranes were developed in ECL substrate for 5 min prior to being imaged on ChemiDoc Imaging System. Compound I treatment, when combined with a CDK4/6i and/or estrogen therapy, prevents the phosphorylation which correlates with the cell cycle arrest in G1 of therapy-resistant cells (FIG. 147K-147L).

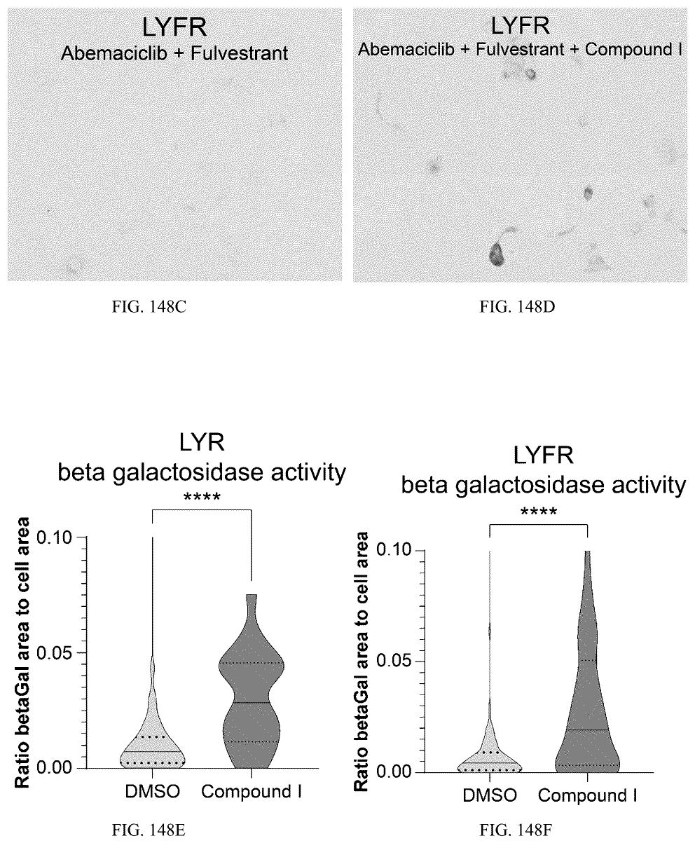

The CDK4/6-/fulvestrant-inhibitor therapy-resistant daughter cells (LYR, LYFR) were then used to analyze senescence-associated beta-galactosidase expression in response to Compound I administered with abemaciclib and/or fulvestrant as compared to DMSO control. Cell cycle arrest after cyclin D1 ablation has previously been demonstrated to be associated with a cellular senescence phenotype (Choi, Y. J. et al. The requirement for cyclin D function in tumor maintenance. Cancer cell. 22:438-451(2012)). Briefly, cells were plated at low density and treated with the agents described for 5 days. A senescence detection kit was used to stain for SA-beta galactosidase (FIG. 148A-148D). Compound I significantly increased beta galactosidase staining in these cells (FIG. 148E-148F), suggesting that Compound I induces cellular senescence following cell cycle arrest in therapy-resistant cells.

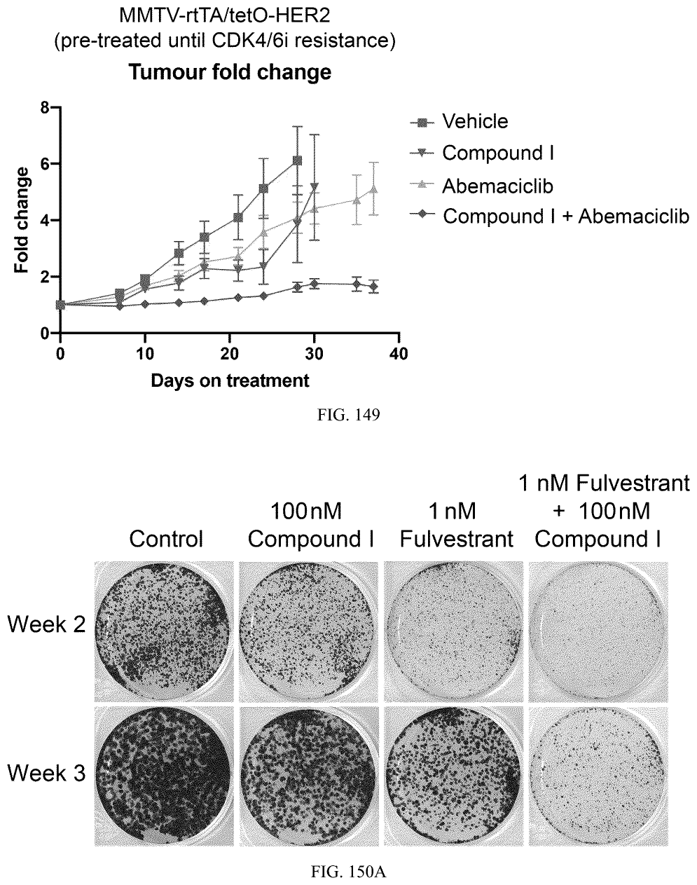

Next, therapy-resistant mice were analyzed for tumor changes over time when administered Compound I in combination with the resistant-therapies (FIG. 149). Breast cancer model mice overexpressing HER2 (MMTV-rtTA/tetO-HER2) were treated with abemaciclib until resistance to treatment was observed. Mice were randomized and dosed with either: 1) Vehicle Control 2) Compound I at 50 mg/kg BID, 3) abemaciclib at 50 mg/kg BID or 4) a combination of Compound I and abemaciclib. Compound I was originally dosed at 75 mg/kg BID but was reduced at day 9. The test agents were delivered at 5 mL/kg by oral gavage. The HER2-overexpressing mice administered Compound I and abemaciclib showed substantially reduced tumor growth relative to either agent administered alone, exhibiting tumor growth inhibition out to day 38 with combination treatment (FIG. 149). These results indicate Compound I can cause in vivo tumor reduction in tumors that have developed therapy resistance in response to prolonged administration of a CDK4/6 inhibitor, suggesting Compound I can re-sensitize tumors to CDK4/6 inhibitor treatment.

### Example 46. Compound I Synergizes with Anti-Estrogen Therapy in ER+ Breast Cancer Cells

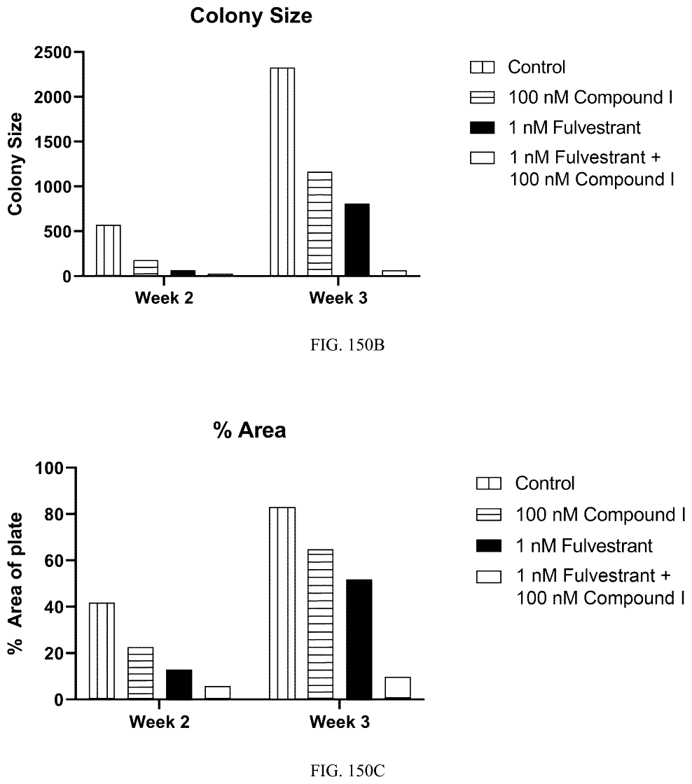

The anti-proliferative effect of Compound I and fulvestrant combination therapy was analyzed in the estrogen receptor-positive (ER+) human T47D breast carcinoma cell line model. The T47D human breast carcinoma cell line was grown in RPMI-1640/1× Glutamax/1× Insulin/10% fetal bovine serum and was plated at 10,000 cells/well in Corning 6 well tissue culture treated plates. Cells were incubated at 37° C. for 24 hours prior to addition of treatment. Two plates were treated with 0.1% DMSO and 300 nM palbociclib in triplicate, two plates were treated with DMSO control across the top row and 100 nM Compound I across the bottom row while two additional plates were treated with 1 nM Fulvestrant across the top row and Fulvestrant+Compound I across the bottom row. Plates were collected at weeks 2 and 3. Collection was performed by washing wells with PBS before incubating 5 minutes with 0.1% crystal violet/20% methanol. Dye was removed and wells washed 4 times with PBS before being scanned at 1200 dpi. Image J was used to count the percent area and colony size for each well and Graph pad was used for analysis. The reduced growth of cells when treated with the combination of fulvestrant and Compound I (FIG. 150-150C) indicate the combination is effective in ER+ breast cancer cells not resistant to CDK4/6 inhibitors.

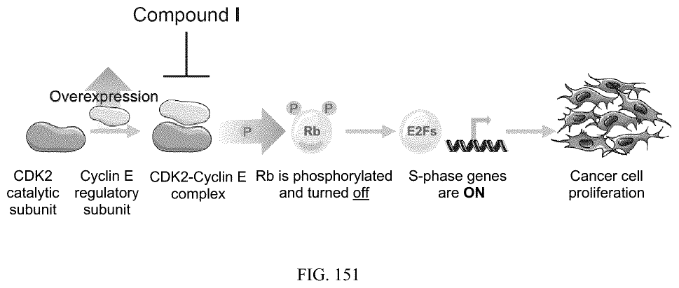

Collectively, these data demonstrate that Compound I is a potent and selective inhibitor of CDK2 capable of inhibiting pRb and inducing G1 cell cycle arrest to inhibit cancer cell proliferation, supporting the development of Compound I as a treatment for cellular proliferation disorders and cancers with dysregulated CDK2 (FIG. 151).

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it can be readily apparent to one of ordinary skill in the art in light of the teaching of this invention that certain changes and modification may be made thereto without departing from the scope of the invention as defined in the claims.

What is claimed is:

1. A method for treating a human patient with an abnormal cellular proliferation, comprising:
(i) obtaining a sample from the human patient;
(ii) detecting whether cyclin E1 (CCNE1) and/or cyclin E2 (CCNE2) are overexpressed and/or amplified in the sample compared with a control sample; and
(iii) if CCNE1 and/or CCNE2 are overexpressed and/or amplified, administering to the human patient an effective amount of a compound of structure: 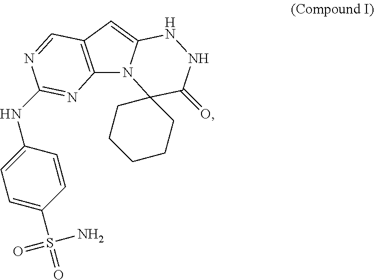 [SMILES: NS(=O)(=O)c1ccc(Nc2ncc3cc4n(c3n2)C2(CCCCC2)C(=O)NN4)cc1]
or a pharmaceutically acceptable salt thereof.

2. The method of claim 1, wherein the abnormal cellular proliferation is selected from the group consisting of ovarian cancer, lung cancer, stomach cancer, and breast cancer.

3. The method of claim 1, wherein the abnormal cellular proliferation is an ovarian cancer.

4. The method of claim 3, wherein the ovarian cancer is an ovarian serous cystadenocarcinoma.

5. The method of claim 1, wherein the abnormal cellular proliferation is a lung cancer.

6. The method of claim 5, wherein the lung cancer is a small cell lung cancer.

7. The method of claim 5, wherein the lung cancer is a lung squamous cell carcinoma.

8. The method of claim 5, wherein the lung cancer is a lung adenocarcinoma.

9. The method of claim 1, wherein the abnormal cellular proliferation is a stomach cancer.

10. The method of claim 9, wherein the stomach cancer is a stomach adenocarcinoma.

11. The method of claim 1, wherein the abnormal cellular proliferation is a breast cancer.

12. The method of claim 11, wherein the breast cancer is a breast invasive carcinoma.

13. The method of claim 11, wherein the breast cancer is estrogen receptor positive (ER+) and human epidermal growth factor receptor 2 negative (HER2−).

14. The method of claim 1, wherein Compound I is administered at a dose from about 100 mg to about 800 mg.

15. The method of claim 14, wherein Compound I is administered once daily.

16. The method of claim 14, wherein Compound I is administered twice daily.
